SEQUENCE LISTING (284 sequences)

SEQ ID NO: 1 - protein, 20 aa, Mus musculus
MARSVTLVFLVLVSLTGLYA

SEQ ID NO: 2 - DNA, 60 bp, Mus musculus
  source 1..60: mol_type = other DNA
atggctcgctcggtgaccctggtctttctggtgcttgtctcactgaccggcctgtatgct

SEQ ID NO: 3 - protein, 8 aa, synthetic construct
  REGION 1..8: note = peptide sequence derived from OVA
SIINFEKL

SEQ ID NO: 4 - DNA, 24 bp, synthetic construct
  misc_feature 1..24: note = DNA sequence derived from OVA
  source 1..24: mol_type = other DNA
tccattataaattttgaaaagttg

SEQ ID NO: 5 - protein, 15 aa, synthetic construct
  REGION 1..15: note = synthesized peptide sequence
GGGASGGGGSGGGGS

SEQ ID NO: 6 - DNA, 45 bp, synthetic construct
  misc_feature 1..45: note = synthesized DNA sequence
  source 1..45: mol_type = other DNA
ggcggaggtgcctctggcggtgggggcagcggtggagggggcagt

SEQ ID NO: 7 - protein, 99 aa, Mus musculus [sequence omitted]

SEQ ID NO: 8 - DNA, 297 bp, Mus musculus
  source 1..297: mol_type = other DNA
atccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatg

SEQ ID NO: 9 - protein, 348 aa, Mus musculus
GPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 10 - DNA, 1044 bp, Mus musculus
  source 1..1044: mol_type = other DNA
ggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcg

SEQ ID NO: 11 - protein, 20 aa, synthetic construct
  REGION 1..20: note = synthesized peptide sequence
GGGGSGGGGSGGGGSGGGGS

SEQ ID NO: 12 - DNA, 60 bp, synthetic construct
  misc_feature 1..60: note = synthesized DNA sequence
  source 1..60: mol_type = other DNA
ggggggggaggctccggtggaggggggtctggagggggggggtctggtggaggcggaagt

SEQ ID NO: 13 - protein, 510 aa, synthetic construct
  REGION 1..510: note = peptide sequence derived from OVA, mus musculus
MARSVTLVFLVLVSLTGLYASIINFEKLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 14 - DNA, 1530 bp, synthetic construct
  misc_feature 1..1530: note = DNA sequence derived from OVA, mus musculus
  source 1..1530: mol_type = other DNA
atggctcgctcggtgaccctggtctttctggtgcttgtctcactgaccggcctgtatgcttccattataaattttgaaaagttgggcggaggtgcctctggcggtgggggcagcggtggagggggcagtatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggggggggaggctccggtggaggggggtctggagggggggggtctggtggaggcggaagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcg

SEQ ID NO: 15 - protein, 236 aa, Mus musculus
MGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGNILTPLLQQDCHQKIDELFSGKLYLIGIAAIVVAVIMIFEMILSMVLCCGIRNSSVY

SEQ ID NO: 16 - DNA, 711 bp, Mus musculus
  source 1..711: mol_type = other DNA
atgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 17 - protein, 746 aa, synthetic construct
  REGION 1..746: note = peptide sequence derived from OVA, mus musculus
MARSVTLVFLVLVSLTGLYASIINFEKLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAMGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGNILTPLLQQDCHQKIDELFSGKLYLIGIAAIVVAVIMIFEMILSMVLCCGIRNSSVY

SEQ ID NO: 18 - DNA, 2241 bp, synthetic construct
  misc_feature 1..2241: note = DNA sequence derived from OVA, mus musculus
  source 1..2241: mol_type = other DNA
atggctcgctcggtgaccctggtctttctggtgcttgtctcactgaccggcctgtatgcttccattataaattttgaaaagttgggcggaggtgcctctggcggtgggggcagcggtggagggggcagtatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggggggggaggctccggtggaggggggtctggagggggggggtctggtggaggcggaagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgatgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 19 - protein, 306 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 20 - DNA, 921 bp, Mus musculus
  source 1..921: mol_type = other DNA
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttag

SEQ ID NO: 21 - protein, 226 aa, Mus musculus
MPVKGGSKCIKYLLFGFNFIFWLAGIAVLAIGLWLRFDSQTKSIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGIAGPLEQFISDTCPKKQLLESFQVKPCPEAISEVFNNKFHIIGAVGIGIAVVMIFGMIFSMILCCAIRRSREMV

SEQ ID NO: 22 - DNA, 681 bp, Mus musculus
  source 1..681: mol_type = other DNA
atgccggtcaaaggaggtagcaagtgcatcaaatacctgctcttcggatttaacttcatcttctggctcgctggcattgcagtgcttgctattggactatggctccgattcgactctcagaccaagagcatcttcgagcaagagaataaccattccagtttctacacaggagtgtacattctgattggagccggggccctcatgatgctggttggtttcctgggctgctgtggagctgtacaagagtcccagtgcatgctgggattgttcttcgggttcctcttggtgatattcgccattgagatagccgccgccgtctggggctatacccacaaggatgaggtgattaaagaactccaggagttttacaaggacacctaccaaaagttacggagcaaggatgaaccccagcgggaaacactcaaagccatccatatggcgttggactgctgtggcatagctggtcctttggagcagtttatctcggacacctgccccaagaaacagcttttggaaagtttccaggttaagccctgccctgaagccatcagtgaggtcttcaacaacaagttccacatcattggagcagtgggtatcggcatcgccgtggtgatgatcttcggcatgatcttcagcatgatcctgtgctgcgccatccgcaggagccgagaaatggtctag

SEQ ID NO: 23 - protein, 532 aa, synthetic construct
  REGION 1..532: note = peptide sequence derived from mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFLMPVKGGSKCIKYLLFGFNFIFWLAGIAVLAIGLWLRFDSQTKSIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGIAGPLEQFISDTCPKKQLLESFQVKPCPEAISEVFNNKFHIIGAVGIGIAVVMIFGMIFSMILCCAIRRSREMV

SEQ ID NO: 24 - DNA, 1599 bp, synthetic construct
  misc_feature 1..1599: note = DNA sequence derived from mus musculus
  source 1..1599: mol_type = other DNA
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttccttatgccggtcaaaggaggtagcaagtgcatcaaatacctgctcttcggatttaacttcatcttctggctcgctggcattgcagtgcttgctattggactatggctccgattcgactctcagaccaagagcatcttcgagcaagagaataaccattccagtttctacacaggagtgtacattctgattggagccggggccctcatgatgctggttggtttcctgggctgctgtggagctgtacaagagtcccagtgcatgctgggattgttcttcgggttcctcttggtgatattcgccattgagatagccgccgccgtctggggctatacccacaaggatgaggtgattaaagaactccaggagttttacaaggacacctaccaaaagttacggagcaaggatgaaccccagcgggaaacactcaaagccatccatatggcgttggactgctgtggcatagctggtcctttggagcagtttatctcggacacctgccccaagaaacagcttttggaaagtttccaggttaagccctgccctgaagccatcagtgaggtcttcaacaacaagttccacatcattggagcagtgggtatcggcatcgccgtggtgatgatcttcggcatgatcttcagcatgatcctgtgctgcgccatccgcaggagccgagaaatggtctag

SEQ ID NO: 25 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 26 - DNA, 447 bp, Mus musculus
  source 1..447: mol_type = other DNA
gcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 27 - protein, 238 aa, Mus musculus
MAVEGGMKCVKFLLYVLLLAFCACAVGLIAIGVAVQVVLKQAITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGASNYTDWENIPGMAKDRVPDSCCINITVGCGNDFKESTIHTQGCVETIAIWLRKNILLVAAAALGIAFVEVLGIIFSCCLVKSIRSGYEVM

SEQ ID NO: 28 - DNA, 717 bp, Mus musculus
  source 1..717: mol_type = other DNA
atggcggtggaaggaggaatgaagtgtgtcaagtttttgctctacgttctcctgctggccttctgcgcctgtgcagtgggattgatcgccattggtgtagcggttcaggttgtcttgaagcaggccattacccatgagactactgctggctcgctgttgcctgtggtcatcattgcagtgggtgccttcctcttcctggtggcctttgtgggctgctgtggggcctgcaaggagaactactgtctcatgattacatttgccatcttcctgtctcttatcatgcttgtggaggtggctgtggccattgctggctatgtgtttagagaccaggtgaagtcagagtttaataaaagcttccagcagcagatgcagaattaccttaaagacaacaaaacagccactattttggacaaattgcagaaagaaaataactgctgtggagcttctaactacacagactgggaaaacatccccggcatggccaaggacagagtccccgattcttgctgcatcaacataactgtgggctgtgggaatgatttcaaggaatccactatccatacccagggctgcgtggagactatagcaatatggctaaggaagaacatactgctggtggctgcagcggccctgggcattgcttttgtggaggtcttgggaattatcttctcctgctgtctggtgaagagtattcgaagtggctatgaagtaatgtag

SEQ ID NO: 29 - protein, 5 aa, synthetic construct
  REGION 1..5: note = synthesized peptide sequence
GGGGS

SEQ ID NO: 30 - DNA, 15 bp, synthetic construct
  misc_feature 1..15: note = synthesized DNA sequence
  source 1..15: mol_type = other DNA
ggaggaggaggaagc

SEQ ID NO: 31 - protein, 397 aa, synthetic construct
  REGION 1..397: note = peptide sequence derived from mus musculus
MAVEGGMKCVKFLLYVLLLAFCACAVGLIAIGVAVQVVLKQAITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGASNYTDWENIPGMAKDRVPDSCCGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSINITVGCGNDFKESTIHTQGCVETIAIWLRKNILLVAAAALGIAFVEVLGIIFSCCLVKSIRSGYEVM

SEQ ID NO: 32 - DNA, 1194 bp, synthetic construct
  misc_feature 1..1194: note = DNA sequence derived from mus musculus
  source 1..1194: mol_type = other DNA
atggcggtggaaggaggaatgaagtgtgtcaagtttttgctctacgttctcctgctggccttctgcgcctgtgcagtgggattgatcgccattggtgtagcggttcaggttgtcttgaagcaggccattacccatgagactactgctggctcgctgttgcctgtggtcatcattgcagtgggtgccttcctcttcctggtggcctttgtgggctgctgtggggcctgcaaggagaactactgtctcatgattacatttgccatcttcctgtctcttatcatgcttgtggaggtggctgtggccattgctggctatgtgtttagagaccaggtgaagtcagagtttaataaaagcttccagcagcagatgcagaattaccttaaagacaacaaaacagccactattttggacaaattgcagaaagaaaataactgctgtggagcttctaactacacagactgggaaaacatccccggcatggccaaggacagagtccccgattcttgctgcggaggaggaggaagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggaggaggaggaagcatcaacataactgtgggctgtgggaatgatttcaaggaatccactatccatacccagggctgcgtggagactatagcaatatggctaaggaagaacatactgctggtggctgcagcggccctgggcattgcttttgtggaggtcttgggaattatcttctcctgctgtctggtgaagagtattcgaagtggctatgaagtaatgtag

SEQ ID NO: 33 - protein, 27 aa, Mus musculus
MALQIPSLLLSAAVVVLMVLSSPGTEG

SEQ ID NO: 34 - DNA, 81 bp, Mus musculus
  source 1..81: mol_type = other DNA
atggctctgcagatccccagcctcctcctctcggctgctgtggtggtgctgatggtgctgagcagcccagggactgagggc

SEQ ID NO: 35 - protein, 17 aa, synthetic construct
  REGION 1..17: note = peptide sequence derived from OVA
ISQAVHAAHAEINEAGR

SEQ ID NO: 36 - DNA, 51 bp, synthetic construct
  misc_feature 1..51: note = DNA sequence derived from OVA
  source 1..51: mol_type = other DNA
atatctcaagctgtccatgcagcacatgcagaaatcaatgaagcaggcaga

SEQ ID NO: 37 - protein, 238 aa, Mus musculus
GDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 38 - DNA, 714 bp, Mus musculus
  source 1..714: mol_type = other DNA
ggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcctggagcaagatgttgagcggcatcgggggctgcgtgcttggggtgatcttcctcgggcttggccttttcatccgtcacaggagtcagaaaggacctcgaggccctcctccagcagggctcctgcag

SEQ ID NO: 39 - protein, 11 aa, synthetic construct
  REGION 1..11: note = synthesized peptide sequence
GGGGSGGGGSG

SEQ ID NO: 40 - DNA, 33 bp, synthetic construct
  misc_feature 1..33: note = synthesized DNA sequence
  source 1..33: mol_type = other DNA
ggaggaggaggaagcggaggaggaggaagcgga

SEQ ID NO: 41 - protein, 293 aa, synthetic construct
  REGION 1..293: note = peptide sequence derived from OVA, mus musculus
MALQIPSLLLSAAVVVLMVLSSPGTEGISQAVHAAHAEINEAGRGGGGSGGGGSGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 42 - DNA, 879 bp, synthetic construct
  misc_feature 1..879: note = DNA sequence derived from OVA, mus musculus
  source 1..879: mol_type = other DNA
atggctctgcagatccccagcctcctcctctcggctgctgtggtggtgctgatggtgctgagcagcccagggactgagggcatatctcaagctgtccatgcagcacatgcagaaatcaatgaagcaggcagaggaggaggaggaagcggaggaggaggaagcggaggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcctggagcaagatgttgagcggcatcgggggctgcgtgcttggggtgatcttcctcgggcttggccttttcatccgtcacaggagtcagaaaggacctcgaggccctcctccagcagggctcctgcag

SEQ ID NO: 43 - protein, 529 aa, synthetic construct
  REGION 1..529: note = peptide sequence derived from OVA, mus musculus
MALQIPSLLLSAAVVVLMVLSSPGTEGISQAVHAAHAEINEAGRGGGGSGGGGSGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQMGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGNILTPLLQQDCHQKIDELFSGKLYLIGIAAIVVAVIMIFEMILSMVLCCGIRNSSVY

SEQ ID NO: 44 - DNA, 1590 bp, synthetic construct
  misc_feature 1..1590: note = DNA sequence derived from OVA, mus musculus
  source 1..1590: mol_type = other DNA
atggctctgcagatccccagcctcctcctctcggctgctgtggtggtgctgatggtgctgagcagcccagggactgagggcatatctcaagctgtccatgcagcacatgcagaaatcaatgaagcaggcagaggaggaggaggaagcggaggaggaggaagcggaggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcctggagcaagatgttgagcggcatcgggggctgcgtgcttggggtgatcttcctcgggcttggccttttcatccgtcacaggagtcagaaaggacctcgaggccctcctccagcagggctcctgcagatgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 45 - protein, 256 aa, Mus musculus
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 46 - DNA, 771 bp, Mus musculus
  source 1..771: mol_type = other DNA
atgccgcgcagcagagctctgattctgggggtcctcgccctgaccaccatgctcagcctctgtggaggtgaagacgacattgaggccgaccacgtaggcacctatggtataagtgtatatcagtctcctggagacattggccagtacacatttgaatttgatggtgatgagttgttctatgtggacttggataagaaggagactgtctggatgcttcctgagtttggccaattggcaagctttgacccccaaggtggactgcaaaacatagctgtagtaaaacacaacttgggagtcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagcaagtcagtcgcagacggtgtttatgagaccagcttcttcgtcaaccgtgactattccttccacaagctgtcttatctcaccttcatcccttctgacgatgacatttatgactgcaaggtggaacactggggcctggaggagccggttctgaaacactgggaacctgagattccagcccccatgtcagagctgacagagactgtggtctgtgccctggggttgtctgtgggccttgtgggcatcgtggtgggcaccatcttcatcattcaaggcctgcgatcaggtggcacctccagacacccagggcctttatga

SEQ ID NO: 47 - protein, 390 aa, synthetic construct
  REGION 1..390: note = peptide sequence derived from mus musculus
MPPSGLRLLPLLLPLPWLLVLTPGRPAAGLSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHSSSDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 48 - DNA, 1173 bp, synthetic construct
  misc_feature 1..1173: note = DNA sequence derived from mus musculus
  source 1..1173: mol_type = other DNA
atgccgccctcggggctgcggctactgccgcttctgctcccactcccgtggcttctagtgctgacgcccgggaggccagccgcgggactctccacctctaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactcttcttctgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 49 - protein, 441 aa, synthetic construct
  REGION 1..441: note = peptide sequence derived from mus musculus
ASGDFCDSSLCLNGGTCLTGQDNDIYCLCPEGFTGLVCNETERGPCSPNPCYNDAKCLVTLDTQRGEIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 50 - DNA, 1326 bp, synthetic construct
  misc_feature 1..1326: note = DNA sequence derived from mus musculus
  source 1..1326: mol_type = other DNA
gcgtctggtgacttctgtgactccagcctgtgcctgaacggtggcacctgcttgacgggccaagacaatgacatctactgcctctgccctgaaggcttcacaggccttgtgtgcaatgagactgagagaggaccatgctccccaaacccttgctacaatgatgccaaatgtctggtgactttggacacacagcgtggggaaatcttcaccgaatacatctgccagtgccctgtgggctactcgggcatccactgtgaaaccgagaccaactactacaacctggatggagaatacatgttcaccacagccgtccccaatactgccgtccccaccccggcccccacccccgatctttccaacaacctagcctcccgttgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggagacaaggagtttttgggtaacctggacaacaacagcctgaaggttaacatgttcaacccgactctggaggcacagtacataaagctgtaccctgtttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaa

SEQ ID NO: 51 - protein, 836 aa, synthetic construct
  REGION 1..836: note = peptide sequence derived from mus musculus
MPPSGLRLLPLLLPLPWLLVLTPGRPAAGLSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHSSSDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCSGGGGSASGDFCDSSLCLNGGTCLTGQDNDIYCLCPEGFTGLVCNETERGPCSPNPCYNDAKCLVTLDTQRGEIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 52 - DNA, 2511 bp, synthetic construct
  misc_feature 1..2511: note = DNA sequence derived from mus musculus
  source 1..2511: mol_type = other DNA
atgccgccctcggggctgcggctactgccgcttctgctcccactcccgtggcttctagtgctgacgcccgggaggccagccgcgggactctccacctctaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactcttcttctgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagcggaggaggaggaagcgcgtctggtgacttctgtgactccagcctgtgcctgaacggtggcacctgcttgacgggccaagacaatgacatctactgcctctgccctgaaggcttcacaggccttgtgtgcaatgagactgagagaggaccatgctccccaaacccttgctacaatgatgccaaatgtctggtgactttggacacacagcgtggggaaatcttcaccgaatacatctgccagtgccctgtgggctactcgggcatccactgtgaaaccgagaccaactactacaacctggatggagaatacatgttcaccacagccgtccccaatactgccgtccccaccccggcccccacccccgatctttccaacaacctagcctcccgttgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggagacaaggagtttttgggtaacctggacaacaacagcctgaaggttaacatgttcaacccgactctggaggcacagtacataaagctgtaccctgtttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaa

SEQ ID NO: 53 - protein, 120 aa, Mus musculus
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 54 - DNA, 363 bp, Mus musculus
  source 1..363: mol_type = other DNA
catatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtag

SEQ ID NO: 55 - protein, 366 aa, synthetic construct
  REGION 1..366: note = peptide sequence derived from mus musculus
MGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGGGSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGGGGSGGNILTPLLQQDCHQKIDELFSGKLYLIGIAAIVVAVIMIFEMILSMVLCCGIRNSSVY

SEQ ID NO: 56 - DNA, 1101 bp, synthetic construct
  misc_feature 1..1101: note = DNA sequence derived from mus musculus
  source 1..1101: mol_type = other DNA
atgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggaggaggaggaagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgggaggaggaggaagcggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 57 - protein, 170 aa, Mus musculus
MAVEGGMKCVKFLLYVLLLAFCACAVGLIAIGVAVQVVLKQAITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGASNYTDWENIPGMAKDRVPDSCC

SEQ ID NO: 58 - DNA, 510 bp, Mus musculus
  source 1..510: mol_type = other DNA
atggcggtggaaggaggaatgaagtgtgtcaagtttttgctctacgttctcctgctggccttctgcgcctgtgcagtgggattgatcgccattggtgtagcggttcaggttgtcttgaagcaggccattacccatgagactactgctggctcgctgttgcctgtggtcatcattgcagtgggtgccttcctcttcctggtggcctttgtgggctgctgtggggcctgcaaggagaactactgtctcatgattacatttgccatcttcctgtctcttatcatgcttgtggaggtggctgtggccattgctggctatgtgtttagagaccaggtgaagtcagagtttaataaaagcttccagcagcagatgcagaattaccttaaagacaacaaaacagccactattttggacaaattgcagaaagaaaataactgctgtggagcttctaactacacagactgggaaaacatccccggcatggccaaggacagagtccccgattcttgctgc

SEQ ID NO: 59 - protein, 68 aa, Mus musculus
INITVGCGNDFKESTIHTQGCVETIAIWLRKNILLVAAAALGIAFVEVLGIIFSCCLVKSIRSGYEVM

SEQ ID NO: 60 - DNA, 207 bp, Mus musculus
  source 1..207: mol_type = other DNA
atcaacataactgtgggctgtgggaatgatttcaaggaatccactatccatacccagggctgcgtggagactatagcaatatggctaaggaagaacatactgctggtggctgcagcggccctgggcattgcttttgtggaggtcttgggaattatcttctcctgctgtctggtgaagagtattcgaagtggctatgaagtaatgtag

SEQ ID NO: 61 - protein, 177 aa, Mus musculus
MGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPS

SEQ ID NO: 62 - DNA, 531 bp, Mus musculus
  source 1..531: mol_type = other DNA
atgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctca

SEQ ID NO: 63 - protein, 59 aa, Mus musculus
GGNILTPLLQQDCHQKIDELFSGKLYLIGIAAIVVAVIMIFEMILSMVLCCGIRNSSVY

SEQ ID NO: 64 - DNA, 180 bp, Mus musculus
  source 1..180: mol_type = other DNA
ggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 65 - protein, 467 aa, synthetic construct
  REGION 1..467: note = peptide sequence derived from mus musculus
IQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 66 - DNA, 1401 bp, synthetic construct
  misc_feature 1..1401: note = DNA sequence derived from mus musculus
  source 1..1401: mol_type = other DNA
atccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggggggggaggctccggtggaggggggtctggagggggggggtctggtggaggcggaagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcg

SEQ ID NO: 67 - protein, 269 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 68 - DNA, 810 bp, Mus musculus
  source 1..810: mol_type = other DNA
gttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttag

SEQ ID NO: 69 - protein, 495 aa, synthetic construct
  REGION 1..495: note = amino acid sequence derived from mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFLMPVKGGSKCIKYLLFGFNFIFWLAGIAVLAIGLWLRFDSQTKSIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGIAGPLEQFISDTCPKKQLLESFQVKPCPEAISEVFNNKFHIIGAVGIGIAVVMIFGMIFSMILCCAIRRSREMV

SEQ ID NO: 70 - DNA, 1488 bp, synthetic construct
  misc_feature 1..1488: note = DNA sequence derived from mus musculus
  source 1..1488: mol_type = other DNA
gttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttccttatgccggtcaaaggaggtagcaagtgcatcaaatacctgctcttcggatttaacttcatcttctggctcgctggcattgcagtgcttgctattggactatggctccgattcgactctcagaccaagagcatcttcgagcaagagaataaccattccagtttctacacaggagtgtacattctgattggagccggggccctcatgatgctggttggtttcctgggctgctgtggagctgtacaagagtcccagtgcatgctgggattgttcttcgggttcctcttggtgatattcgccattgagatagccgccgccgtctggggctatacccacaaggatgaggtgattaaagaactccaggagttttacaaggacacctaccaaaagttacggagcaaggatgaaccccagcgggaaacactcaaagccatccatatggcgttggactgctgtggcatagctggtcctttggagcagtttatctcggacacctgccccaagaaacagcttttggaaagtttccaggttaagccctgccctgaagccatcagtgaggtcttcaacaacaagttccacatcattggagcagtgggtatcggcatcgccgtggtgatgatcttcggcatgatcttcagcatgatcctgtgctgcgccatccgcaggagccgagaaatggtctag

SEQ ID NO: 71 - protein, 233 aa, Mus musculus
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 72 - DNA, 702 bp, Mus musculus
  source 1..702: mol_type = other DNA
gaagacgacattgaggccgaccacgtaggcacctatggtataagtgtatatcagtctcctggagacattggccagtacacatttgaatttgatggtgatgagttgttctatgtggacttggataagaaggagactgtctggatgcttcctgagtttggccaattggcaagctttgacccccaaggtggactgcaaaacatagctgtagtaaaacacaacttgggagtcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagcaagtcagtcgcagacggtgtttatgagaccagcttcttcgtcaaccgtgactattccttccacaagctgtcttatctcaccttcatcccttctgacgatgacatttatgactgcaaggtggaacactggggcctggaggagccggttctgaaacactgggaacctgagattccagcccccatgtcagagctgacagagactgtggtctgtgccctggggttgtctgtgggccttgtgggcatcgtggtgggcaccatcttcatcattcaaggcctgcgatcaggtggcacctccagacacccagggcctttatga

SEQ ID NO: 73 - protein, 361 aa, Mus musculus
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHSSSDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 74 - DNA, 1086 bp, Mus musculus
  source 1..1086: mol_type = other DNA
ctctccacctctaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactcttcttctgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 75 - protein, 807 aa, synthetic construct
  REGION 1..807: note = amino acid sequence derived from mus musculus
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHSSSDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCSGGGGSASGDFCDSSLCLNGGTCLTGQDNDIYCLCPEGFTGLVCNETERGPCSPNPCYNDAKCLVTLDTQRGEIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 76 - DNA, 2424 bp, synthetic construct
  misc_feature 1..2424: note = DNA sequence derived from mus musculus
  source 1..2424: mol_type = other DNA
ctctccacctctaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactcttcttctgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagcggaggaggaggaagcgcgtctggtgacttctgtgactccagcctgtgcctgaacggtggcacctgcttgacgggccaagacaatgacatctactgcctctgccctgaaggcttcacaggccttgtgtgcaatgagactgagagaggaccatgctccccaaacccttgctacaatgatgccaaatgtctggtgactttggacacacagcgtggggaaatcttcaccgaatacatctgccagtgccctgtgggctactcgggcatccactgtgaaaccgagaccaactactacaacctggatggagaatacatgttcaccacagccgtccccaatactgccgtccccaccccggcccccacccccgatctttccaacaacctagcctcccgttgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggagacaaggagtttttgggtaacctggacaacaacagcctgaaggttaacatgttcaacccgactctggaggcacagtacataaagctgtaccctgtttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaa

SEQ ID NO: 77 - protein, 17 aa, synthetic construct
  REGION 1..17: note = synthesized amino acid sequence
TSGGSGGTGGSGGTGGS

SEQ ID NO: 78 - DNA, 51 bp, synthetic construct
  misc_feature 1..51: note = synthesized DNA sequence
  source 1..51: mol_type = other DNA
accagcggaggaagcggtggcacaggcgggtctggaggcaccggaggctca

SEQ ID NO: 79 - protein, 14 aa, synthetic construct
  REGION 1..14: note = synthesized amino acid sequence
NYHLENEVARLKKL

SEQ ID NO: 80 - DNA, 42 bp, synthetic construct
  misc_feature 1..42: note = synthesized DNA sequence
  source 1..42: mol_type = other DNA
aattaccacctggagaacgaagtggccaggctgaagaaactc

SEQ ID NO: 81 - protein, 274 aa, synthetic construct
  REGION 1..274: note = amino acid sequence derived from mus musculus
MALPVTALLLPLALLLHAARPEQKLISEEDLDAVVTQESALTSSPGETVTLTCRSSTGAVTTSNYASWVQEKPDHLFTGLIGGTNNRAPGVPARFSGSLIGDKAALTITGAQTEDEAIYFCVLWYSDHWVFGGGTKLTVLGGGGGSGGGGSGGGGSGGGGSDVQLQESGPGLVAPSQSLSITCTVSGFLLTDYGVNWVRQSPGKGLEWLGVIWGDGITDYNSALKSRLSVTKDNSKSQVFLKMNSLQSGDSARYYCVTGLFDYWGQGTTLTVSS

SEQ ID NO: 82 - DNA, 822 bp, synthetic construct
  misc_feature 1..822: note = DNA sequence derived from mus musculus
  source 1..822: mol_type = other DNA
atggccttaccagtgaccgccttgctcctgccgctggccttgctgctccacgccgccaggccggagcagaagctgatcagcgaggaggacctggacgccgttgtgacccaggaatccgctctgacctcttctccaggcgaaaccgtgactctgacttgccgtagtagcaccggggctgtgaccacatctaactatgccagttgggtccaggaaaaaccggatcacctgtttactggcctgattggcggcaccaacaatcgcgcaccgggtgtgcccgctcgtttcagcggttccctgattggggacaaggcagcactgactatcaccggcgcccagaccgaagatgaggcgatctatttttgcgtcctgtggtacagcgaccattgggtgttcgggggaggcaccaaactgacagtgctgggcggaggaggaggttcaggaggaggaggtagcgggggaggcggttccgggggaggcggttctgatgtgcagctgcaagaatccgggccaggactggttgcgccttctcagagtctgtcaattacatgtactgttagtggctttctgctgaccgactatggtgtgaactgggttcgtcagagcccaggcaagggtctggagtggctgggagtgatttggggggatggaatcacagactacaatagcgcactgaaatctcggctgagtgttaccaaagataacagcaagtcccaggtcttcctgaagatgaacagcctgcaaagcggcgactccgctcgctattactgcgttaccggactgtttgattattgggggcaggggacaactctgactgtttcctcc

SEQ ID NO: 83 - protein, 253 aa, synthetic construct
  REGION 1..253: note = amino acid sequence derived from mus musculus
EQKLISEEDLDAVVTQESALTSSPGETVTLTCRSSTGAVTTSNYASWVQEKPDHLFTGLIGGTNNRAPGVPARFSGSLIGDKAALTITGAQTEDEAIYFCVLWYSDHWVFGGGTKLTVLGGGGGSGGGGSGGGGSGGGGSDVQLQESGPGLVAPSQSLSITCTVSGFLLTDYGVNWVRQSPGKGLEWLGVIWGDGITDYNSALKSRLSVTKDNSKSQVFLKMNSLQSGDSARYYCVTGLFDYWGQGTTLTVSS

SEQ ID NO: 84 - DNA, 759 bp, synthetic construct
  misc_feature 1..759: note = DNA sequence derived from mus musculus
  source 1..759: mol_type = other DNA
gagcagaagctgatcagcgaggaggacctggacgccgttgtgacccaggaatccgctctgacctcttctccaggcgaaaccgtgactctgacttgccgtagtagcaccggggctgtgaccacatctaactatgccagttgggtccaggaaaaaccggatcacctgtttactggcctgattggcggcaccaacaatcgcgcaccgggtgtgcccgctcgtttcagcggttccctgattggggacaaggcagcactgactatcaccggcgcccagaccgaagatgaggcgatctatttttgcgtcctgtggtacagcgaccattgggtgttcgggggaggcaccaaactgacagtgctgggcggaggaggaggttcaggaggaggaggtagcgggggaggcggttccgggggaggcggttctgatgtgcagctgcaagaatccgggccaggactggttgcgccttctcagagtctgtcaattacatgtactgttagtggctttctgctgaccgactatggtgtgaactgggttcgtcagagcccaggcaagggtctggagtggctgggagtgatttggggggatggaatcacagactacaatagcgcactgaaatctcggctgagtgttaccaaagataacagcaagtcccaggtcttcctgaagatgaacagcctgcaaagcggcgactccgctcgctattactgcgttaccggactgtttgattattgggggcaggggacaactctgactgtttcctcc

SEQ ID NO: 85 - protein, 220 aa, Mus musculus
KPQAPELRIFPKKMDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 86 - DNA, 660 bp, Mus musculus
  source 1..660: mol_type = other DNA
aagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaagcgtgtttgcaaatgtcccaggccgctagtcagacaggaaggcaagcccagaccttcagagaaaattgtg

SEQ ID NO: 87 - protein, 730 aa, synthetic construct
  REGION 1..730: note = amion acid sequence derived from mus musculus
MALPVTALLLPLALLLHAARPEQKLISEEDLDAVVTQESALTSSPGETVTLTCRSSTGAVTTSNYASWVQEKPDHLFTGLIGGTNNRAPGVPARFSGSLIGDKAALTITGAQTEDEAIYFCVLWYSDHWVFGGGTKLTVLGGGGGSGGGGSGGGGSGGGGSDVQLQESGPGLVAPSQSLSITCTVSGFLLTDYGVNWVRQSPGKGLEWLGVIWGDGITDYNSALKSRLSVTKDNSKSQVFLKMNSLQSGDSARYYCVTGLFDYWGQGTTLTVSSKPQAPELRIFPKKMDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIVMGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGNILTPLLQQDCHQKIDELFSGKLYLIGIAAIVVAVIMIFEMILSMVLCCGIRNSSVY

SEQ ID NO: 88 - DNA, 2193 bp, synthetic construct
  misc_feature 1..2193: note = DNA sequence derived from mus musculus
  source 1..2193: mol_type = other DNA
atggccttaccagtgaccgccttgctcctgccgctggccttgctgctccacgccgccaggccggagcagaagctgatcagcgaggaggacctggacgccgttgtgacccaggaatccgctctgacctcttctccaggcgaaaccgtgactctgacttgccgtagtagcaccggggctgtgaccacatctaactatgccagttgggtccaggaaaaaccggatcacctgtttactggcctgattggcggcaccaacaatcgcgcaccgggtgtgcccgctcgtttcagcggttccctgattggggacaaggcagcactgactatcaccggcgcccagaccgaagatgaggcgatctatttttgcgtcctgtggtacagcgaccattgggtgttcgggggaggcaccaaactgacagtgctgggcggaggaggaggttcaggaggaggaggtagcgggggaggcggttccgggggaggcggttctgatgtgcagctgcaagaatccgggccaggactggttgcgccttctcagagtctgtcaattacatgtactgttagtggctttctgctgaccgactatggtgtgaactgggttcgtcagagcccaggcaagggtctggagtggctgggagtgatttggggggatggaatcacagactacaatagcgcactgaaatctcggctgagtgttaccaaagataacagcaagtcccaggtcttcctgaagatgaacagcctgcaaagcggcgactccgctcgctattactgcgttaccggactgtttgattattgggggcaggggacaactctgactgtttcctccaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaagcgtgtttgcaaatgtcccaggccgctagtcagacaggaaggcaagcccagaccttcagagaaaattgtgatgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 89 - protein, 709 aa, synthetic construct
  REGION 1..709: note = amino acid sequence derived from mus musculus
EQKLISEEDLDAVVTQESALTSSPGETVTLTCRSSTGAVTTSNYASWVQEKPDHLFTGLIGGTNNRAPGVPARFSGSLIGDKAALTITGAQTEDEAIYFCVLWYSDHWVFGGGTKLTVLGGGGGSGGGGSGGGGSGGGGSDVQLQESGPGLVAPSQSLSITCTVSGFLLTDYGVNWVRQSPGKGLEWLGVIWGDGITDYNSALKSRLSVTKDNSKSQVFLKMNSLQSGDSARYYCVTGLFDYWGQGTTLTVSSKPQAPELRIFPKKMDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIVMGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGNILTPLLQQDCHQKIDELFSGKLYLIGIAAIVVAVIMIFEMILSMVLCCGIRNSSVY

SEQ ID NO: 90 - DNA, 2130 bp, synthetic construct
  misc_feature 1..2130: note = DNA sequence derived from mus musculus
  source 1..2130: mol_type = other DNA
gagcagaagctgatcagcgaggaggacctggacgccgttgtgacccaggaatccgctctgacctcttctccaggcgaaaccgtgactctgacttgccgtagtagcaccggggctgtgaccacatctaactatgccagttgggtccaggaaaaaccggatcacctgtttactggcctgattggcggcaccaacaatcgcgcaccgggtgtgcccgctcgtttcagcggttccctgattggggacaaggcagcactgactatcaccggcgcccagaccgaagatgaggcgatctatttttgcgtcctgtggtacagcgaccattgggtgttcgggggaggcaccaaactgacagtgctgggcggaggaggaggttcaggaggaggaggtagcgggggaggcggttccgggggaggcggttctgatgtgcagctgcaagaatccgggccaggactggttgcgccttctcagagtctgtcaattacatgtactgttagtggctttctgctgaccgactatggtgtgaactgggttcgtcagagcccaggcaagggtctggagtggctgggagtgatttggggggatggaatcacagactacaatagcgcactgaaatctcggctgagtgttaccaaagataacagcaagtcccaggtcttcctgaagatgaacagcctgcaaagcggcgactccgctcgctattactgcgttaccggactgtttgattattgggggcaggggacaactctgactgtttcctccaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaagcgtgtttgcaaatgtcccaggccgctagtcagacaggaaggcaagcccagaccttcagagaaaattgtgatgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 91 - protein, 732 aa, synthetic construct
  REGION 1..732: note = sc-Dimer-CD81-IL-12a
MALQIPSLLLSAAVVVLMVLSSPGTEGISQAVHAAHAEINEAGRGGGGSGGGGSGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQMGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGNILTPLLQQDCHQKIDELFSGKLYLIGIAAIVVAVIMIFEMILSMVLCCGIRNSSVY

SEQ ID NO: 92 - DNA, 2238 bp, synthetic construct
  misc_feature 1..2238: note = sc-dimer-CD81-IL-12a
  source 1..2238: mol_type = other DNA
atggctctgcagatccccagcctcctcctctcggctgctgtggtggtgctgatggtgctgagcagcccagggactgagggcatatctcaagctgtccatgcagcacatgcagaaatcaatgaagcaggcagaggaggaggaggaagcggaggaggaggaagcggaggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcctggagcaagatgttgagcggcatcgggggctgcgtgcttggggtgatcttcctcgggcttggccttttcatccgtcacaggagtcagaaaggacctcgaggccctcctccagcagggctcctgcagatgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggaggaggaggaagcagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggaggaggaagcggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactgagtgctgtgctgtggcatccggaacagctccgtgtactga

SEQ ID NO: 93 - protein, 193 aa, synthetic construct
  REGION 1..193: note = Il-12a
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 94 - DNA, 579 bp, synthetic construct
  misc_feature 1..579: note = IL-12a
  source 1..579: mol_type = other DNA
agggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 95 - protein, 439 aa, synthetic construct
  REGION 1..439: note = CD81-IL12a
MGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGNILTPLLQQDCHQKIDELFSGKLYLIGIAAIVVAVIMIFEMILSMVLCCGIRNSSVY

SEQ ID NO: 96 - DNA, 1320 bp, synthetic construct
  misc_feature 1..1320: note = CD81-IL12a
  source 1..1320: mol_type = other DNA
atgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggaggaggaggaagcagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggaggaggaagcggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 97 - protein, 335 aa, synthetic construct
  REGION 1..335: note = IL-12b
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 98 - DNA, 1008 bp, synthetic construct
  misc_feature 1..1008: note = IL-12b
  source 1..1008: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 99 - protein, 187 aa, synthetic construct
  REGION 1..187: note = IL-6
FPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 100 - DNA, 561 bp, synthetic construct
  misc_feature 1..561: note = IL-6
  source 1..561: mol_type = other DNA
ttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacc

SEQ ID NO: 101 - protein, 433 aa, synthetic construct
  REGION 1..433: note = CD81-IL6
MGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGGGSFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQTGGGGSGGNILTPLLQQDCHQKIDELFSGKLYLIGIAAIVVAVIMIFEMILSMVLCCGIRNSSVY

SEQ ID NO: 102 - DNA, 1302 bp, synthetic construct
  misc_feature 1..1302: note = CD81-IL6
  source 1..1302: mol_type = other DNA
atgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggaggaggaggaagcttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaaccggaggaggaggaagcggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 103 - protein, 288 aa, synthetic construct
  REGION 1..288: note = hCD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 104 - DNA, 864 bp, synthetic construct
  misc_feature 1..864: note = hCD81
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 105 - protein, 228 aa, synthetic construct
  REGION 1..228: note = hCD9
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 106 - DNA, 687 bp, synthetic construct
  misc_feature 1..687: note = hCD9
  source 1..687: mol_type = other DNA
atgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctag

SEQ ID NO: 107 - protein, 392 aa, synthetic construct
  REGION 1..392: note = hCD80-hCD9
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPVMPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEI

SEQ ID NO: 108 - DNA, 1551 bp, synthetic construct
  misc_feature 1..1551: note = hCD80-hCD9
  source 1..1551: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtaatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctag

SEQ ID NO: 109 - protein, 133 aa, synthetic construct [sequence omitted]

SEQ ID NO: 110 - DNA, 399 bp, synthetic construct
  misc_feature 1..399: note = hIL-2
  source 1..399: mol_type = other DNA
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 111 - protein, 170 aa, synthetic construct
  REGION 1..170: note = hCD63 1-170
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCC

SEQ ID NO: 112 - DNA, 510 bp, synthetic construct
  misc_feature 1..510: note = hCD63 1-170
  source 1..510: mol_type = other DNA
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgc

SEQ ID NO: 113 - protein, 68 aa, synthetic construct
  REGION 1..68: note = hCD63 171-238
INVTVGCGINFNEKAIHKEGCVEKIGGWLRKNVLVVAAAALGIAFVEVLGIVFACCLVKSIRSGYEVM

SEQ ID NO: 114 - DNA, 207 bp, synthetic construct
  misc_feature 1..207: note = hCD63 171-238
  source 1..207: mol_type = other DNA
attaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtag

SEQ ID NO: 115 - protein, 381 aa, synthetic construct
  REGION 1..381: note = hCD63-IL2
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSINVTVGCGINFNEKAIHKEGCVEKIGGWLRKNVLVVAAAALGIAFVEVLGIVFACCLVKSIRSGYEVM

SEQ ID NO: 116 - DNA, 1146 bp, synthetic construct
  misc_feature 1..1146: note = hCD63-IL2
  source 1..1146: mol_type = other DNA
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcggaggaggaggaagcgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggaggaggaagcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtag

SEQ ID NO: 117 - protein, 20 aa, synthetic construct
  REGION 1..20: note = hb2mg signal
MSRSVALAVLALLSLSGLEA

SEQ ID NO: 118 - DNA, 60 bp, synthetic construct
  misc_feature 1..60: note = hb2mg signal
  source 1..60: mol_type = other DNA
atgtctcgctccgtggccttagctgtgctcgcgctactctctctttctggcctggaggct

SEQ ID NO: 119 - protein, 9 aa, synthetic construct
  REGION 1..9: note = WT1
CYTWNQMNL

SEQ ID NO: 120 - DNA, 27 bp, synthetic construct
  misc_feature 1..27: note = WT1
  source 1..27: mol_type = other DNA
tgctacacctggaaccagatgaacctg

SEQ ID NO: 121 - protein, 99 aa, synthetic construct [sequence omitted]

SEQ ID NO: 122 - DNA, 297 bp, synthetic construct
  misc_feature 1..297: note = hb2mg
  source 1..297: mol_type = other DNA
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 123 - protein, 341 aa, synthetic construct
  REGION 1..341: note = hMHCIa
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 124 - DNA, 1023 bp, synthetic construct
  misc_feature 1..1023: note = hMHCIa
  source 1..1023: mol_type = other DNA
ggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcaccaagcgcaagtgggaggcggcccatgtggcggagcagcagagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagccatcttcccagcccaccgtccccatcgtgggcatcattgctggcctggttctccttggagctgtgatcactggagctgtggtcgctgctgtgatgtggaggaggaacagctcagatagaaaaggagggagctactctcaggctgcaagcagtgacagtgcccagggctctgatgtgtctctcacagcttgtaaagtg

SEQ ID NO: 125 - protein, 460 aa, synthetic construct
  REGION 1..460: note = hsMHCI
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 126 - DNA, 1380 bp, synthetic construct
  misc_feature 1..1380: note = hsMHCI
  source 1..1380: mol_type = other DNA
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggggggggaggctccggtggaggggggtctggagggggggggtctggtggaggcggaagtggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcaccaagcgcaagtgggaggcggcccatgtggcggagcagcagagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagccatcttcccagcccaccgtccccatcgtgggcatcattgctggcctggttctccttggagctgtgatcactggagctgtggtcgctgctgtgatgtggaggaggaacagctcagatagaaaaggagggagctactctcaggctgcaagcagtgacagtgcccagggctctgatgtgtctctcacagcttgtaaagtg

SEQ ID NO: 127 - protein, 504 aa, synthetic construct
  REGION 1..504: note = hsc-Trimer
MSRSVALAVLALLSLSGLEACYTWNQMNLGGGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 128 - DNA, 1512 bp, synthetic construct
  misc_feature 1..1512: note = hsc-Trimer
  source 1..1512: mol_type = other DNA
atgtctcgctccgtggccttagctgtgctcgcgctactctctctttctggcctggaggcttgctacacctggaaccagatgaacctgggcggaggtgcctctggcggtgggggcagcggtggagggggcagtatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggggggggaggctccggtggaggggggtctggagggggggggtctggtggaggcggaagtggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcaccaagcgcaagtgggaggcggcccatgtggcggagcagcagagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagccatcttcccagcccaccgtccccatcgtgggcatcattgctggcctggttctccttggagctgtgatcactggagctgtggtcgctgctgtgatgtggaggaggaacagctcagatagaaaaggagggagctactctcaggctgcaagcagtgacagtgcccagggctctgatgtgtctctcacagcttgtaaagtg

SEQ ID NO: 129 - protein, 236 aa, synthetic construct
  REGION 1..236: note = hCD81
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 130 - DNA, 711 bp, synthetic construct
  misc_feature 1..711: note = hCD81
  source 1..711: mol_type = other DNA
atgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactga

SEQ ID NO: 131 - protein, 740 aa, synthetic construct
  REGION 1..740: note = hsc-Trimer-CD81
MSRSVALAVLALLSLSGLEACYTWNQMNLGGGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKVMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 132 - DNA, 2223 bp, synthetic construct
  misc_feature 1..2223: note = hsc-Trimer-CD81
  source 1..2223: mol_type = other DNA
atgtctcgctccgtggccttagctgtgctcgcgctactctctctttctggcctggaggcttgctacacctggaaccagatgaacctgggcggaggtgcctctggcggtgggggcagcggtggagggggcagtatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggggggggaggctccggtggaggggggtctggagggggggggtctggtggaggcggaagtggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcaccaagcgcaagtgggaggcggcccatgtggcggagcagcagagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagccatcttcccagcccaccgtccccatcgtgggcatcattgctggcctggttctccttggagctgtgatcactggagctgtggtcgctgctgtgatgtggaggaggaacagctcagatagaaaaggagggagctactctcaggctgcaagcagtgacagtgcccagggctctgatgtgtctctcacagcttgtaaagtgatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactga

SEQ ID NO: 133 - protein, 395 aa, synthetic construct
  REGION 1..395: note = CD81-IL-2
MGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGNILTPLLQQDCHQKIDELFSGKLYLIGIAAIVVAVIMIFEMILSMVLCCGIRNSSVY

SEQ ID NO: 134 - DNA, 1188 bp, synthetic construct
  misc_feature 1..1188: note = CD81-IL2
  source 1..1188: mol_type = other DNA
atgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggaggaggaggaagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggaggaggaggaagcggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 135 - protein, 905 aa, synthetic construct
  REGION 1..905: note = sc-Trimer-CD81-IL-2
MARSVTLVFLVLVSLTGLYASIINFEKLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAMGVEGCTKCIKYLLFVFNFVFWLAGGVILGVALWLRHDPQTTSLLYLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNALTTLTTTILRNSLCPSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGNILTPLLQQDCHQKIDELFSGKLYLIGIAAIVVAVIMIFEMILSMVLCCGIRNSSVY

SEQ ID NO: 136 - DNA, 2718 bp, synthetic construct
  misc_feature 1..2718: note = sc-Trimer-CD81-IL-2
  source 1..2718: mol_type = other DNA
atggctcgctcggtgaccctggtctttctggtgcttgtctcactgaccggcctgtatgcttccattataaattttgaaaagttgggcggaggtgcctctggcggtgggggcagcggtggagggggcagtatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggggggggaggctccggtggaggggggtctggagggggggggtctggtggaggcggaagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgatgggggtggagggctgcaccaaatgcatcaaatacctgctcttcgtcttcaatttcgtcttctggctggctggaggcgtgatcctaggtgtagctctgtggttgcgtcatgatccacagaccaccagcctgctgtacctggaactgggaaacaaaccggcacccaacaccttctacgtgggcatctacattctcattgctgtgggagctgtgatgatgtttgtaggcttcctggggtgctatggggccatccaggagtcccagtgtctgctggggacgttcttcacctgccttgtgatcctgtttgcctgtgaggtggctgcaggcatctggggcttcgtaaacaaagaccagatcgccaaggatgtgaagcagttctatgaccaggcccttcagcaagctgtgatggatgatgatgccaacaatgccaaggctgtggtgaagactttccatgagacgctcaactgttgtggctccaacgcactgaccacactgactaccaccatactgaggaacagcctgtgtccctcaggaggaggaggaagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggaggaggaggaagcggcggcaacatactcacccccttactgcagcaagattgtcatcagaaaatcgatgagctcttctctgggaagctgtacctcattggaattgcagccattgtggtagctgtcattatgatctttgagatgattctgagcatggtgctgtgctgtggcatccggaacagctccgtgtactga

SEQ ID NO: 137 - protein, 550 aa, synthetic construct
  REGION 1..550: note = Aka-Luc
MEDAKNIKKGPAPFYPLEDGTAGEQLHKAMKRYALVPGAIAFTDAHIQVDVTYAEYFEMSVRLAEAMRRYGLNTNHRIVVCSENSSQFFMPVLGALFIGVAVAPANDIYNERELLNSMGISQPTVVFVSKKGLRKVLNVQKKLPIIRKIIIMDSKTDYQGFQSMYTFVTSHLPPSFNEYDFVPESFDRDKTIALIMNSSGSTGLPKGVALPHRTACVRFSHARDPIFGYQNIPDTAILSVVPFHHGFGMFTTLGYLICGFRVVLMYRFEEELFLRSLQDYKIQSALLVPTLFSCLAKSTLIDKYDLSSLREIASGGAPLSKEVGEAVAKRFRLPGIRQGYGLTETTNAVMITPEGDRKPGSVGKVVPFFEAKVVDLVTGKTLGVNQRGELCVRGPMIMSGYVNNPEATNALIDKDGWLHSGDIAYWDEDEHFFIVDRLKSLIKYKGYQVAPAELEGILLQHPYIFDAGVAGLPDDDAGELPAAVVVLEHGKTMTEKEIVDYVASQVTTAKKLRGGVVFVDEVPRGSTGKLDARKIREILTKAKKDGKIAV

SEQ ID NO: 138 - DNA, 1650 bp, synthetic construct
  misc_feature 1..1650: note = Aka-Luc
  source 1..1650: mol_type = other DNA
atggaagatgccaaaaacattaagaagggcccagcgccgttctacccactcgaagacgggaccgccggcgagcagctgcacaaagccatgaagcgctacgccctggtgcccggcgccatcgcctttaccgacgcacatattcaggtggacgttacctacgccgagtacttcgagatgagcgttcggctggcagaagctatgaggcgctatgggctgaatacaaaccatcggatcgtggtgtgcagcgagaatagctcgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcgaaaggtcctcaacgtgcaaaagaagctaccgatcatacgaaagatcatcatcatggatagcaagaccgactaccagggcttccaaagcatgtacaccttcgtgacttcccatttgccacccagcttcaacgagtacgacttcgtgcccgagagcttcgaccgggacaaaaccatcgccctgatcatgaacagtagtggtagtacaggattacccaagggcgtagccctaccgcaccgcaccgcttgtgtccgattcagtcatgcccgcgaccccatcttcggctaccagaacatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagctgcctcgctaagagcactctcatcgacaagtacgacctaagcagcttgcgcgagatcgccagcggcggggcgccgctcagcaaggaggtaggtgaggccgtggccaaacgcttccgcctaccaggcatccgccagggctatggcctgacagaaacaaccaacgccgtcatgatcacccccgagggggaccgtaagcctggctcagtaggcaaggtggtgcccttcttcgaggctaaggtggtagacttggtcaccggtaagacactgggtgtgaaccagcgcggtgagctgtgcgtccgtggccccatgatcatgagcggctacgttaacaaccccgaggctacgaacgctctcatcgacaaggacggctggctgcacagcggcgacatcgcctactgggacgaggacgagcacttcttcatcgtggaccggctgaagagcctgatcaaatacaagggctaccaggtagccccagccgaactggagggcatcctgctgcaacacccctacatcttcgacgccggagtcgccggcctgcccgacgacgatgccggcgagctgcccgccgcagtcgtcgtgttggaacacggtaaaaccatgaccgagaaagagatcgtggactatgtggccagccaggttacaaccgccaagaagctgcgcggtggtgttgtgtttgtggatgaagtccctagaggatcgaccggcaagttagacgcccgcaagatccgcgagattctcactaaggccaagaaggacggcaagatcgccgtg

SEQ ID NO: 139 - protein, 798 aa, synthetic construct
  REGION 1..798: note = CD63-Aka-Luc
MAVEGGMKCVKFLLYVLLLAFCACAVGLIAIGVAVQVVLKQAITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGASNYTDWENIPGMAKDRVPDSCCGGGGSMEDAKNIKKGPAPFYPLEDGTAGEQLHKAMKRYALVPGAIAFTDAHIQVDVTYAEYFEMSVRLAEAMRRYGLNTNHRIVVCSENSSQFFMPVLGALFIGVAVAPANDIYNERELLNSMGISQPTVVFVSKKGLRKVLNVQKKLPIIRKIIIMDSKTDYQGFQSMYTFVTSHLPPSFNEYDFVPESFDRDKTIALIMNSSGSTGLPKGVALPHRTACVRFSHARDPIFGYQNIPDTAILSVVPFHHGFGMFTTLGYLICGFRVVLMYRFEEELFLRSLQDYKIQSALLVPTLFSCLAKSTLIDKYDLSSLREIASGGAPLSKEVGEAVAKRFRLPGIRQGYGLTETTNAVMITPEGDRKPGSVGKVVPFFEAKVVDLVTGKTLGVNQRGELCVRGPMIMSGYVNNPEATNALIDKDGWLHSGDIAYWDEDEHFFIVDRLKSLIKYKGYQVAPAELEGILLQHPYIFDAGVAGLPDDDAGELPAAVVVLEHGKTMTEKEIVDYVASQVTTAKKLRGGVVFVDEVPRGSTGKLDARKIREILTKAKKDGKIAVGGGGSINITVGCGNDFKESTIHTQGCVETIAIWLRKNILLVAAAALGIAFVEVLGIIFSCCLVKSIRSGYEVM

SEQ ID NO: 140 - DNA, 2397 bp, synthetic construct
  misc_feature 1..2397: note = CD63-Aka-Luc
  source 1..2397: mol_type = other DNA
atggcggtggaaggaggaatgaagtgtgtcaagtttttgctctacgttctcctgctggccttctgcgcctgtgcagtgggattgatcgccattggtgtagcggttcaggttgtcttgaagcaggccattacccatgagactactgctggctcgctgttgcctgtggtcatcattgcagtgggtgccttcctcttcctggtggcctttgtgggctgctgtggggcctgcaaggagaactactgtctcatgattacatttgccatcttcctgtctcttatcatgcttgtggaggtggctgtggccattgctggctatgtgtttagagaccaggtgaagtcagagtttaataaaagcttccagcagcagatgcagaattaccttaaagacaacaaaacagccactattttggacaaattgcagaaagaaaataactgctgtggagcttctaactacacagactgggaaaacatccccggcatggccaaggacagagtccccgattcttgctgcggtggtggtggttctatggaagatgccaaaaacattaagaagggcccagcgccgttctacccactcgaagacgggaccgccggcgagcagctgcacaaagccatgaagcgctacgccctggtgcccggcgccatcgcctttaccgacgcacatattcaggtggacgttacctacgccgagtacttcgagatgagcgttcggctggcagaagctatgaggcgctatgggctgaatacaaaccatcggatcgtggtgtgcagcgagaatagctcgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcgaaaggtcctcaacgtgcaaaagaagctaccgatcatacgaaagatcatcatcatggatagcaagaccgactaccagggcttccaaagcatgtacaccttcgtgacttcccatttgccacccagcttcaacgagtacgacttcgtgcccgagagcttcgaccgggacaaaaccatcgccctgatcatgaacagtagtggtagtacaggattacccaagggcgtagccctaccgcaccgcaccgcttgtgtccgattcagtcatgcccgcgaccccatcttcggctaccagaacatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagctgcctcgctaagagcactctcatcgacaagtacgacctaagcagcttgcgcgagatcgccagcggcggggcgccgctcagcaaggaggtaggtgaggccgtggccaaacgcttccgcctaccaggcatccgccagggctatggcctgacagaaacaaccaacgccgtcatgatcacccccgagggggaccgtaagcctggctcagtaggcaaggtggtgcccttcttcgaggctaaggtggtagacttggtcaccggtaagacactgggtgtgaaccagcgcggtgagctgtgcgtccgtggccccatgatcatgagcggctacgttaacaaccccgaggctacgaacgctctcatcgacaaggacggctggctgcacagcggcgacatcgcctactgggacgaggacgagcacttcttcatcgtggaccggctgaagagcctgatcaaatacaagggctaccaggtagccccagccgaactggagggcatcctgctgcaacacccctacatcttcgacgccggagtcgccggcctgcccgacgacgatgccggcgagctgcccgccgcagtcgtcgtgttggaacacggtaaaaccatgaccgagaaagagatcgtggactatgtggccagccaggttacaaccgccaagaagctgcgcggtggtgttgtgtttgtggatgaagtccctagaggatcgaccggcaagttagacgcccgcaagatccgcgagattctcactaaggccaagaaggacggcaagatcgccgtgggtggtggtggttctatcaacataactgtgggctgtgggaatgatttcaaggaatccactatccatacccagggctgcgtggagactatagcaatatggctaaggaagaacatactgctggtggctgcagcggccctgggcattgcttttgtggaggtcttgggaattatcttctcctgctgtctggtgaagagtattcgaagtggctatgaagtaatgtag

SEQ ID NO: 141 - protein, 9 aa, synthetic construct
  REGION 1..9: note = SAR-CoV2 peptide
KLWAQCVQL

SEQ ID NO: 142 - DNA, 27 bp, synthetic construct
  misc_feature 1..27: note = SARS-CoV2 DNA
  source 1..27: mol_type = other DNA
aaactgtgggcccagtgtgtgcagctg

SEQ ID NO: 143 - protein, 341 aa, synthetic construct
  REGION 1..341: note = HLA-A0201
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 144 - DNA, 1023 bp, synthetic construct
  misc_feature 1..1023: note = HLA-A0201
  source 1..1023: mol_type = other DNA
ggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtg

SEQ ID NO: 145 - protein, 460 aa, synthetic construct
  REGION 1..460: note = hsMHC-I
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 146 - DNA, 1380 bp, synthetic construct
  misc_feature 1..1380: note = hsMHC-I
  source 1..1380: mol_type = other DNA
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggcgggtctggcggcggcggcagtggcggtggcggtagcggcggaggtggatctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtg

SEQ ID NO: 147 - protein, 504 aa, synthetic construct
  REGION 1..504: note = SARS-CoV2sc-Trimer
MSRSVALAVLALLSLSGLEAKLWAQCVQLGGGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 148 - DNA, 1512 bp, synthetic construct
  misc_feature 1..1512: note = SARS-CoV2sc-Trimer
  source 1..1512: mol_type = other DNA
atgtctcgctccgtggccttagctgtgctcgcgctactctctctttctggcctggaggctaaactgtgggcccagtgtgtgcagctgggcggaggggcatcaggcggcggtgggtcaggtggaggtgggagtatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggcgggtctggcggcggcggcagtggcggtggcggtagcggcggaggtggatctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtg

SEQ ID NO: 149 - protein, 740 aa, synthetic construct
  REGION 1..740: note = SARS-CoV2sc-Trimer-CD81
MSRSVALAVLALLSLSGLEAKLWAQCVQLGGGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 150 - DNA, 2223 bp, synthetic construct
  misc_feature 1..2223: note = SARS-CoV2sc-Trimer-CD81
  source 1..2223: mol_type = other DNA
atgtctcgctccgtggccttagctgtgctcgcgctactctctctttctggcctggaggctaaactgtgggcccagtgtgtgcagctgggcggaggggcatcaggcggcggtgggtcaggtggaggtgggagtatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggcgggtctggcggcggcggcagtggcggtggcggtagcggcggaggtggatctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactga

SEQ ID NO: 151 - protein, 29 aa, synthetic construct
  REGION 1..29: note = HLA DR1beta signal
MVCLKLPGGSCMTALTVTLMVLSSPLALS

SEQ ID NO: 152 - DNA, 87 bp, synthetic construct
  misc_feature 1..87: note = HLA DR1beta signal
  source 1..87: mol_type = other DNA
atggtgtgccttaaactccctggcggaagctgcatgaccgccttgactgtgaccctgatggttctgagctctccattggccctgagt

SEQ ID NO: 153 - protein, 15 aa, synthetic construct
  REGION 1..15: note = TPI
GELIGILNAAKVPAD

SEQ ID NO: 154 - DNA, 45 bp, synthetic construct
  misc_feature 1..45: note = TPI
  source 1..45: mol_type = other DNA
ggagagctcattggcatcctgaacgctgccaaggtgcccgcagac

SEQ ID NO: 155 - protein, 11 aa, synthetic construct
  REGION 1..11: note = linker
GGGGSGGGGSG

SEQ ID NO: 156 - DNA, 33 bp, synthetic construct
  misc_feature 1..33: note = linker
  source 1..33: mol_type = other DNA
ggtggcggagggagcggaggcggtgggtccggc

SEQ ID NO: 157 - protein, 237 aa, synthetic construct
  REGION 1..237: note = HLA DR1beta
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 158 - DNA, 711 bp, synthetic construct
  misc_feature 1..711: note = HLA DR1beta
  source 1..711: mol_type = other DNA
ggggacactagaccacgatttctttggcagttgaagttcgagtgccacttcttcaatgggacagagagggtcaggcttctggaacgctgcatctacaaccaagaggagagtgtgcggtttgactctgatgtgggcgaatatcgggcagtcacggaacttgggcgtcctgatgccgagtactggaattcccagaaggatctgctggaacaacgacgggctgctgttgacacgtactgtcgccacaactatggcgtcggtgagagctttaccgtgcaaaggagagtagagcccaaggtgaccgtttatccctccaagacccaacccttgcagcatcacaatctgctggtatgtagcgtgtctggcttctatcctggcagcatagaagtcaggtggtttcggaacggtcaggaagagaaagctggggtggttagcacaggactcattcagaatggggactggacattccagaccctggtgatgctggagacagtaccgagatcaggagaggtgtacacatgtcaggtggaacatccctcagtgactagtccactcactgtcgaatggcgtgcgagatccgagtcagcacagagcaaaatgctctccggagttggcggattcgtcctgggactgctgtttctcggtgccggtctgttcatctacttccgcaaccagaagggccattccggcctgcagcctactgggtttctgtct

SEQ ID NO: 159 - protein, 236 aa, synthetic construct; identical to SEQ ID NO: 129
  REGION 1..236: note = hCD81

SEQ ID NO: 160 - DNA, 708 bp, synthetic construct
  misc_feature 1..708: note = hCD81
  source 1..708: mol_type = other DNA
atgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtac

SEQ ID NO: 161 - protein, 22 aa, synthetic construct
  REGION 1..22: note = P2A
GSGATNFSLLKQAGDVEENPGP

SEQ ID NO: 162 - DNA, 66 bp, synthetic construct
  misc_feature 1..66: note = P2A
  source 1..66: mol_type = other DNA
ggatctggagccacgaacttctctctgttaaagcaagcaggagacgtggaagaaaaccccggtcct

SEQ ID NO: 163 - protein, 254 aa, synthetic construct
  REGION 1..254: note = HLA DR1alpha
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 164 - DNA, 765 bp, synthetic construct
  misc_feature 1..765: note = HLA DR1alpha
  source 1..765: mol_type = other DNA
atggccattagcggggttccagtgctgggcttcttcatcatagcggtactgatgtctgcccaggaatcctgggcgatcaaagaagaacacgtcatcatacaggcagagttctacctgaatcccgatcagtccggagagtttatgttcgactttgatggggacgagattttccatgtggatatggctaagaaggaaaccgtctggagactggaagagtttggccgttttgccagttttgaggcccaaggagcactggccaacatagcagtcgacaaagccaacctggagattatgacgaaacgcagcaactatacgcccattaccaatgtaccacctgaagtgacagtgctgactaactcacccgtggaattgcgagaacccaatgtgctgatctgcttcatcgacaagttcactccaccggttgttaacgtcacttggcttcggaatggcaagcctgtcactaccggtgtctctgagacagtgtttctgccgagggaggatcatctctttcgcaagttccactatcttccctttctccctagtaccgaagatgtatacgattgcagagtggagcattggggattggacgagccactgctgaagcactgggagttcgacgcaccttcaccccttccagagacaaccgaaaatgtggtgtgtgctctcggactcacagttggcttggtgggcattatcattgggaccattttcatcatcaaaggggtgaggaaaagcaacgctgctgagcggagaggtcctctgtga

SEQ ID NO: 165 - protein, 804 aa, synthetic construct
  REGION 1..804: note = HLADR-1sc-TPI1-hCD81
MVCLKLPGGSCMTALTVTLMVLSSPLALSGELIGILNAAKVPADGGGGSGGGGSGGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLSMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYGSGATNFSLLKQAGDVEENPGPMAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 166 - DNA, 2415 bp, synthetic construct
  misc_feature 1..2415: note = HLADR-1sc-TPI1-hCD81
  source 1..2415: mol_type = other DNA
atggtgtgccttaaactccctggcggaagctgcatgaccgccttgactgtgaccctgatggttctgagctctccattggccctgagtggagagctcattggcatcctgaacgctgccaaggtgcccgcagacggtggcggagggagcggaggcggtgggtccggcggggacactagaccacgatttctttggcagttgaagttcgagtgccacttcttcaatgggacagagagggtcaggcttctggaacgctgcatctacaaccaagaggagagtgtgcggtttgactctgatgtgggcgaatatcgggcagtcacggaacttgggcgtcctgatgccgagtactggaattcccagaaggatctgctggaacaacgacgggctgctgttgacacgtactgtcgccacaactatggcgtcggtgagagctttaccgtgcaaaggagagtagagcccaaggtgaccgtttatccctccaagacccaacccttgcagcatcacaatctgctggtatgtagcgtgtctggcttctatcctggcagcatagaagtcaggtggtttcggaacggtcaggaagagaaagctggggtggttagcacaggactcattcagaatggggactggacattccagaccctggtgatgctggagacagtaccgagatcaggagaggtgtacacatgtcaggtggaacatccctcagtgactagtccactcactgtcgaatggcgtgcgagatccgagtcagcacagagcaaaatgctctccggagttggcggattcgtcctgggactgctgtttctcggtgccggtctgttcatctacttccgcaaccagaagggccattccggcctgcagcctactgggtttctgtctatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtacggatctggagccacgaacttctctctgttaaagcaagcaggagacgtggaagaaaaccccggtcctatggccattagcggggttccagtgctgggcttcttcatcatagcggtactgatgtctgcccaggaatcctgggcgatcaaagaagaacacgtcatcatacaggcagagttctacctgaatcccgatcagtccggagagtttatgttcgactttgatggggacgagattttccatgtggatatggctaagaaggaaaccgtctggagactggaagagtttggccgttttgccagttttgaggcccaaggagcactggccaacatagcagtcgacaaagccaacctggagattatgacgaaacgcagcaactatacgcccattaccaatgtaccacctgaagtgacagtgctgactaactcacccgtggaattgcgagaacccaatgtgctgatctgcttcatcgacaagttcactccaccggttgttaacgtcacttggcttcggaatggcaagcctgtcactaccggtgtctctgagacagtgtttctgccgagggaggatcatctctttcgcaagttccactatcttccctttctccctagtaccgaagatgtatacgattgcagagtggagcattggggattggacgagccactgctgaagcactgggagttcgacgcaccttcaccccttccagagacaaccgaaaatgtggtgtgtgctctcggactcacagttggcttggtgggcattatcattgggaccattttcatcatcaaaggggtgaggaaaagcaacgctgctgagcggagaggtcctctgtga

SEQ ID NO: 167 - protein, 328 aa, synthetic construct
  REGION 1..328: note = IL-12beta
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 168 - DNA, 984 bp, synthetic construct
  misc_feature 1..984: note = IL-12beta
  source 1..984: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 169 - protein, 15 aa, synthetic construct
  REGION 1..15: note = linker
GGGGSGGGGSGGGGS

SEQ ID NO: 170 - DNA, 45 bp, synthetic construct
  misc_feature 1..45: note = linker
  source 1..45: mol_type = other DNA
ggaggaggcgggtctggcggcggagggagcggtggcggtgggtcc

SEQ ID NO: 171 - protein, 197 aa, synthetic construct
  REGION 1..197: note = IL-12alpha
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 172 - DNA, 591 bp, synthetic construct
  misc_feature 1..591: note = IL-12alpha
  source 1..591: mol_type = other DNA
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 173 - protein, 17 aa, synthetic construct
  REGION 1..17: note = linker
TSGGSGGTGGSGGTGGS

SEQ ID NO: 174 - DNA, 51 bp, synthetic construct
  misc_feature 1..51: note = linker
  source 1..51: mol_type = other DNA
accagcggaggctctggagggacaggtgggagtggtggcactggaggctcc

SEQ ID NO: 175 - protein, 364 aa, synthetic construct
  REGION 1..364: note = MFGE8
LDICSKNPCHNGGLCEEISQEVRGEVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 176 - DNA, 1095 bp, synthetic construct
  misc_feature 1..1095: note = MFGE8
  source 1..1095: mol_type = other DNA
ctggatatctgttccaaaaacccctgccacaacggtggtttatgcgaggagatttcccaagaagtgcgaggagaagtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttga

SEQ ID NO: 177 - protein, 921 aa, synthetic construct
  REGION 1..921: note = hIL-12sc-MFGe8
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASTSGGSGGTGGSGGTGGSLDICSKNPCHNGGLCEEISQEVRGEVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 178 - DNA, 2766 bp, synthetic construct
  misc_feature 1..2766: note = hIL-12sc-MFGe8
  source 1..2766: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggaggaggcgggtctggcggcggagggagcggtggcggtgggtccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccaccagcggaggctctggagggacaggtgggagtggtggcactggaggctccctggatatctgttccaaaaacccctgccacaacggtggtttatgcgaggagatttcccaagaagtgcgaggagaagtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttga

SEQ ID NO: 179 - protein, 259 aa, synthetic construct
  REGION 1..259: note = TCRbeta
MGSRLLCWVLLCLLGAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTYHGTGYFGEGSWLTVVEDLNKVFPPEVAVFEPSEAEISHTQKATLVCLATGFFPDHVELSWWVNGKEVHSGVCTDPQPLKEQPALNDSRYALSSRLRVSATFWQNPRNHFRCQVQFYGLSENDEWTQDRAKPVTQIVSAEAWGRAD

SEQ ID NO: 180 - DNA, 777 bp, synthetic construct
  misc_feature 1..777: note = TCRbeta
  source 1..777: mol_type = other DNA
atgggcagcagattgctctgttgggtgctgctctgtttgcttggtgccggtccagtcaacgctggagtaacccagactcctaagttccgcattctgaagataggccaatccatgaccctgcagtgtacccaggacatgaaccacaactacatgtactggtatcggcaagatccaggcatgggtctgaagctgatctactatagcgttggtgcagggattacggacaaaggcgaggtgcccaatggctacaacgtgagtcgttccactaccgaggactttcccctcagattggaactggcagctccatcacagacatccgtctacttctgcgcatctacataccatggaaccgggtatttcggcgaaggtagctggcttaccgtggttgaggacctcaacaaggtgtttccgcctgaggtagccgtctttgagccgtctgaagcggagatcagccatactcagaaagccacactggtgtgcttggcaactgggttctttcccgatcatgtggagctctcttggtgggtcaatgggaaggaggtgcacagtggagtgtgcactgatccacaacctctcaaagagcagccagcgctgaatgatagccggtatgccctgtcaagcaggcttagagtttcagccacattctggcagaatcctcgcaaccactttaggtgtcaggtccagttctatggcctgtccgaaaatgacgaatggacgcaagaccgagccaaacccgtgacacagatcgtcagtgccgaagcttggggaagagctgat

SEQ ID NO: 181 - protein, 22 aa, synthetic construct
  REGION 1..22: note = P2A
GSGATNFSLLKQAGDVEENPGP

SEQ ID NO: 182 - DNA, 66 bp, synthetic construct; identical to SEQ ID NO: 162
  misc_feature 1..66: note = P2A
  source 1..66: mol_type = other DNA

SEQ ID NO: 183 - protein, 223 aa, synthetic construct
  REGION 1..223: note = TCRalpha
MEKMLECAFIVLWLQLGWLSGIQVEQSPPDLILQEGANSTLRCNFSDSVNNLQWFHQNPWGQLINLFYIPSGTKQNGRLSATTVATERYSLLYISSSQTTDSGVYFCAALIQGAQKLVFGQGTRLTINPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 184 - DNA, 669 bp, synthetic construct
  misc_feature 1..669: note = TCRalpha
  source 1..669: mol_type = other DNA
atggagaaaatgcttgagtgtgccttcattgtgctgtggctgcaacttgggtggctgtcagggatacaagtggaacagagtccacccgatttgatcctccaggaaggagccaattccacccttcgatgcaactttagcgattcagtgaacaacctccagtggttccaccagaatccctggggtcagctgatcaatctgttctacattccttctggcaccaagcagaatgggcgtctttccgccacaactgtcgcaacagagcgctattctctgctgtacatcagcagttcccagactaccgattctggcgtctacttttgtgccgcattgattcaaggagcacagaagctcgtgtttgggcaaggcacaaggctgaccattaaccccaacatacagaacccagatcctgctgtgtatcagctgagagacagcaaaagtagcgataagtcagtatgcctgttcacagactttgactctcagactaatgtgagccaatccaaagattccgacgtctatatcaccgacaaatgcgttctggacatgcggtcaatggacttcaagagcaattctgctgttgcttggagtaacaagtccgactttgcctgtgccaatgcgttcaacaatagcatcattccggaggatacgttctttccaagccctgagtcatct

SEQ ID NO: 185 - protein, 22 aa, synthetic construct
  REGION 1..22: note = P2A
GSGATNFSLLKQAGDVEENPGP

SEQ ID NO: 186 - DNA, 66 bp, synthetic construct; identical to SEQ ID NO: 162
  misc_feature 1..66: note = P2A
  source 1..66: mol_type = other DNA

SEQ ID NO: 187 - protein, 239 aa, synthetic construct
  REGION 1..239: note = Venus
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 188 - DNA, 720 bp, synthetic construct
  misc_feature 1..720: note = Venus
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 189 - protein, 765 aa, synthetic construct
  REGION 1..765: note = TPI-1 TCR + Venus
MGSRLLCWVLLCLLGAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTYHGTGYFGEGSWLTVVEDLNKVFPPEVAVFEPSEAEISHTQKATLVCLATGFFPDHVELSWWVNGKEVHSGVCTDPQPLKEQPALNDSRYALSSRLRVSATFWQNPRNHFRCQVQFYGLSENDEWTQDRAKPVTQIVSAEAWGRADGSGATNFSLLKQAGDVEENPGPMEKMLECAFIVLWLQLGWLSGIQVEQSPPDLILQEGANSTLRCNFSDSVNNLQWFHQNPWGQLINLFYIPSGTKQNGRLSATTVATERYSLLYISSSQTTDSGVYFCAALIQGAQKLVFGQGTRLTINPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGSGATNFSLLKQAGDVEENPGPMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 190 - DNA, 2298 bp, synthetic construct
  misc_feature 1..2298: note = TPI-1 TCR + Venus
  source 1..2298: mol_type = other DNA
atgggcagcagattgctctgttgggtgctgctctgtttgcttggtgccggtccagtcaacgctggagtaacccagactcctaagttccgcattctgaagataggccaatccatgaccctgcagtgtacccaggacatgaaccacaactacatgtactggtatcggcaagatccaggcatgggtctgaagctgatctactatagcgttggtgcagggattacggacaaaggcgaggtgcccaatggctacaacgtgagtcgttccactaccgaggactttcccctcagattggaactggcagctccatcacagacatccgtctacttctgcgcatctacataccatggaaccgggtatttcggcgaaggtagctggcttaccgtggttgaggacctcaacaaggtgtttccgcctgaggtagccgtctttgagccgtctgaagcggagatcagccatactcagaaagccacactggtgtgcttggcaactgggttctttcccgatcatgtggagctctcttggtgggtcaatgggaaggaggtgcacagtggagtgtgcactgatccacaacctctcaaagagcagccagcgctgaatgatagccggtatgccctgtcaagcaggcttagagtttcagccacattctggcagaatcctcgcaaccactttaggtgtcaggtccagttctatggcctgtccgaaaatgacgaatggacgcaagaccgagccaaacccgtgacacagatcgtcagtgccgaagcttggggaagagctgatggatctggagccacgaacttctctctgttaaagcaagcaggagacgtggaagaaaaccccggtcctatggagaaaatgcttgagtgtgccttcattgtgctgtggctgcaacttgggtggctgtcagggatacaagtggaacagagtccacccgatttgatcctccaggaaggagccaattccacccttcgatgcaactttagcgattcagtgaacaacctccagtggttccaccagaatccctggggtcagctgatcaatctgttctacattccttctggcaccaagcagaatgggcgtctttccgccacaactgtcgcaacagagcgctattctctgctgtacatcagcagttcccagactaccgattctggcgtctacttttgtgccgcattgattcaaggagcacagaagctcgtgtttgggcaaggcacaaggctgaccattaaccccaacatacagaacccagatcctgctgtgtatcagctgagagacagcaaaagtagcgataagtcagtatgcctgttcacagactttgactctcagactaatgtgagccaatccaaagattccgacgtctatatcaccgacaaatgcgttctggacatgcggtcaatggacttcaagagcaattctgctgttgcttggagtaacaagtccgactttgcctgtgccaatgcgttcaacaatagcatcattccggaggatacgttctttccaagccctgagtcatctggatctggagccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 191 - protein, 509 aa, synthetic construct
  REGION 1..509: note = sc-Trimer
MARSVTLVFLVLVSLTGLYASIINFEKLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVERPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 192 - DNA, 1530 bp, synthetic construct; identical to SEQ ID NO: 14
  misc_feature 1..1530: note = sc-Trimer
  source 1..1530: mol_type = other DNA

SEQ ID NO: 193 - protein, 21 aa, synthetic construct
  REGION 1..21: note = T2A
GSGEGRGSLLTCGDVEENPGP

SEQ ID NO: 194 - DNA, 63 bp, synthetic construct
  misc_feature 1..63: note = T2A
  source 1..63: mol_type = other DNA
ggctccggcgagggcaggggaagtcttctaacatgcggggacgtggaggaaaatcccggccca

SEQ ID NO: 195 - protein, 169 aa, synthetic construct
  REGION 1..169: note = IL-2
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 196 - DNA, 507 bp, synthetic construct
  misc_feature 1..507: note = IL-2
  source 1..507: mol_type = other DNA
atgtactcaatgcagctggctagttgtgtgaccctgaccctcgtgctgctcgtgaacagcgccccaacctcaagctctacctccagtagcacagccgaagctcagcagcagcagcaacagcagcagcagcagcagcagcacctggagcagctgctgatggacctgcaggagctgctgagccggatggagaactacaggaacctgaagctgcctaggatgctgacattcaagttctacctgccaaagcaggccaccgagctgaaggacctgcagtgtctggaggacgagctgggccccctgcgccacgtgctcgacctgacacagtccaagtccttccagctggaggacgcagagaacttcatctccaacatcagagtgaccgtggtgaaactgaagggctctgacaacacctttgagtgtcagttcgacgacgagagtgccactgtggtggatttcctgaggcggtggatcgctttctgccagagcattatctctaccagcccacag

SEQ ID NO: 197 - protein, 5 aa, synthetic construct
  REGION 1..5: note = linker
GGGGS

SEQ ID NO: 198 - DNA, 15 bp, synthetic construct
  misc_feature 1..15: note = linker
  source 1..15: mol_type = other DNA
ggtggcggcggaagc

SEQ ID NO: 199 - protein, 117 aa, synthetic construct
  REGION 1..117: note = CD8 partial
VISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 200 - DNA, 351 bp, synthetic construct
  misc_feature 1..351: note = CD8 partial
  source 1..351: mol_type = other DNA
gtcatcagcaacagcgtgatgtatttctcctctgtggtgcctgtgctgcagaaggtgaacagcaccaccaccaagcctgtgctgaggactccaagccccgtgcaccccactggtactagccagcctcagcgccccgaggactgtagacctagaggatctgtgaaaggaacaggcctggactttgcatgtgacatctatatttgggcaccacttgccggcatttgcgtggccctgctgctgtccctgatcatcacactgatctgctaccacaggagccgaaagcgtgtttgcaaatgtcccaggccgctagtcagacaggaaggcaagcccagaccttcagagaaaattgtg

SEQ ID NO: 201 - protein, 22 aa, synthetic construct
  REGION 1..22: note = P2A
GSGATNFSLLKQAGDVEENPGP

SEQ ID NO: 202 - DNA, 66 bp, synthetic construct
  misc_feature 1..66: note = P2A
  source 1..66: mol_type = other DNA
ggttcaggggctaccaacttttccctccttaagcaggccggagatgtcgaggagaatcctggccct

SEQ ID NO: 203 - protein, 306 aa, synthetic construct; identical to SEQ ID NO: 19
  REGION 1..306: note = CD80

SEQ ID NO: 204 - DNA, 921 bp, synthetic construct
  misc_feature 1..921: note = CD80
  source 1..921: mol_type = other DNA
atggcgtgtaattgccagctcatgcaggacacacctctgttgaaattcccgtgtccaaggcttatcctgctgttcgtgctcttgatacgactcagccaggtgtcaagcgacgttgatgagcagctgtctaagagcgtgaaggataaggtgttgctgccctgtcggtacaatagcccacatgaggacgagtcagaggatcgcatctattggcagaaacacgacaaagtggtgctgagcgtgatcgctggcaagctgaaagtttggcccgagtacaagaaccggacactgtacgacaataccacatactccctgattattctggggctcgtgctctccgatagaggcacttatagctgcgttgtacagaagaaggaaagagggacttatgaggttaaacaccttgctctggtgaagctgagtatcaaggctgacttctccacgccaaatataaccgaatcaggaaatcctagtgccgatactaaacgcatcacttgttttgccagtggtgggttccccaaaccgagattctcttggctggaaaacggaagggagttgcccggcattaacaccaccatttctcaagaccccgaatccgagctttacaccattagcagccaacttgacttcaacacaacacggaaccacaccatcaagtgtctgatcaagtatggcgacgcacatgtcagtgaggatttcacatgggagaaaccacccgaagatcctccagactccaagaacactctcgtgctgtttggtgcaggatttggagccgtaataaccgtagtggtcattgtcgtcatcatcaagtgcttctgcaaacaccgttcttgctttcgacgcaatgaagcctctagggaaacaaacaactctctgacttttggccctgaagaagccctggcagagcaaacggtctttctgtag

SEQ ID NO: 205 - protein, 1150 aa, synthetic construct
  REGION 1..1150: note = sc-Trimer-T2A-IL-2-CD8-P2A-CD80
MARSVTLVFLVLVSLTGLYASIINFEKLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAGSGEGRGSLLTCGDVEENPGPMYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIVGSGATNFSLLKQAGDVEENPGPMACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 206 - DNA, 3453 bp, synthetic construct
  misc_feature 1..3453: note = sc-Trimer-T2A-IL-2-CD8-P2A-CD80
  source 1..3453: mol_type = other DNA
atggctcgctcggtgaccctggtctttctggtgcttgtctcactgaccggcctgtatgcttccattataaattttgaaaagttgggcggaggtgcctctggcggtgggggcagcggtggagggggcagtatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggggggggaggctccggtggaggggggtctggagggggggggtctggtggaggcggaagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgggctccggcgagggcaggggaagtcttctaacatgcggggacgtggaggaaaatcccggcccaatgtactcaatgcagctggctagttgtgtgaccctgaccctcgtgctgctcgtgaacagcgccccaacctcaagctctacctccagtagcacagccgaagctcagcagcagcagcaacagcagcagcagcagcagcagcacctggagcagctgctgatggacctgcaggagctgctgagccggatggagaactacaggaacctgaagctgcctaggatgctgacattcaagttctacctgccaaagcaggccaccgagctgaaggacctgcagtgtctggaggacgagctgggccccctgcgccacgtgctcgacctgacacagtccaagtccttccagctggaggacgcagagaacttcatctccaacatcagagtgaccgtggtgaaactgaagggctctgacaacacctttgagtgtcagttcgacgacgagagtgccactgtggtggatttcctgaggcggtggatcgctttctgccagagcattatctctaccagcccacagggtggcggcggaagcgtcatcagcaacagcgtgatgtatttctcctctgtggtgcctgtgctgcagaaggtgaacagcaccaccaccaagcctgtgctgaggactccaagccccgtgcaccccactggtactagccagcctcagcgccccgaggactgtagacctagaggatctgtgaaaggaacaggcctggactttgcatgtgacatctatatttgggcaccacttgccggcatttgcgtggccctgctgctgtccctgatcatcacactgatctgctaccacaggagccgaaagcgtgtttgcaaatgtcccaggccgctagtcagacaggaaggcaagcccagaccttcagagaaaattgtgggttcaggggctaccaacttttccctccttaagcaggccggagatgtcgaggagaatcctggccctatggcgtgtaattgccagctcatgcaggacacacctctgttgaaattcccgtgtccaaggcttatcctgctgttcgtgctcttgatacgactcagccaggtgtcaagcgacgttgatgagcagctgtctaagagcgtgaaggataaggtgttgctgccctgtcggtacaatagcccacatgaggacgagtcagaggatcgcatctattggcagaaacacgacaaagtggtgctgagcgtgatcgctggcaagctgaaagtttggcccgagtacaagaaccggacactgtacgacaataccacatactccctgattattctggggctcgtgctctccgatagaggcacttatagctgcgttgtacagaagaaggaaagagggacttatgaggttaaacaccttgctctggtgaagctgagtatcaaggctgacttctccacgccaaatataaccgaatcaggaaatcctagtgccgatactaaacgcatcacttgttttgccagtggtgggttccccaaaccgagattctcttggctggaaaacggaagggagttgcccggcattaacaccaccatttctcaagaccccgaatccgagctttacaccattagcagccaacttgacttcaacacaacacggaaccacaccatcaagtgtctgatcaagtatggcgacgcacatgtcagtgaggatttcacatgggagaaaccacccgaagatcctccagactccaagaacactctcgtgctgtttggtgcaggatttggagccgtaataaccgtagtggtcattgtcgtcatcatcaagtgcttctgcaaacaccgttcttgctttcgacgcaatgaagcctctagggaaacaaacaactctctgacttttggccctgaagaagccctggcagagcaaacggtctttctgtag

SEQ ID NO: 207 - protein, 236 aa, synthetic construct; identical to SEQ ID NO: 15
  REGION 1..236: note = CD81

SEQ ID NO: 208 - DNA, 711 bp, synthetic construct; identical to SEQ ID NO: 16
  misc_feature 1..711: note = CD81
  source 1..711: mol_type = other DNA

SEQ ID NO: 209 - protein, 386 aa, synthetic construct
  REGION 1..386: note = OVA
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 210 - DNA, 1161 bp, synthetic construct
  misc_feature 1..1161: note = OVA
  source 1..1161: mol_type = other DNA
atgggctccatcggcgcagcaagcatggaattttgttttgatgtattcaaggagctcaaagtccaccatgccaatgagaacatcttctactgccccattgccatcatgtcagctctagccatggtatacctgggtgcaaaagacagcaccaggacacagataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaa

SEQ ID NO: 211 - protein, 21 aa, synthetic construct
  REGION 1..21: note = T2A
GSGEGRGSLLTCGDVEENPGP

SEQ ID NO: 212 - protein, 22 aa, synthetic construct
  REGION 1..22: note = T2A
GSGATNFSLLKQAGDVEENPGP

SEQ ID NO: 213 - protein, 23 aa, synthetic construct
  REGION 1..23: note = B2A
GSGQCTNYALLKLAGDVESNPGP

SEQ ID NO: 214 - protein, 25 aa, synthetic construct
  REGION 1..25: note = F2A
GSGVKQTLNFDLLKLAGDVESNPGP

SEQ ID NO: 215 - protein, 20 aa, synthetic construct
MARSVTLVFLVLVSLTGLYA

SEQ ID NO: 216 - DNA, 60 bp, synthetic construct
  source 1..60: mol_type = other DNA
atggccaggtctgtgacactggtgtttctggtgctggtgtccctgacaggactctacgcc

SEQ ID NO: 217 - protein, 8 aa, synthetic construct
SIINFEKL

SEQ ID NO: 218 - DNA, 24 bp, synthetic construct
  source 1..24: mol_type = other DNA
tctatcatcaatttcgaaaaactg

SEQ ID NO: 219 - protein, 15 aa, synthetic construct
GGGASGGGGSGGGGS

SEQ ID NO: 220 - DNA, 45 bp, synthetic construct
  source 1..45: mol_type = other DNA
gggggcggtgccagcgggggaggcggctccggcgggggcggctcc

SEQ ID NO: 221 - protein, 99 aa, synthetic construct [sequence omitted]

SEQ ID NO: 222 - DNA, 297 bp, synthetic construct
  source 1..297: mol_type = other DNA
atccagaagaccccccagatccaggtctactctcgacaccctcctgagaacggcaagccaaacatcctgaactgctatgtgacccagttccatcccccacacattgagatccagatgctgaagaacggcaagaagatcccaaaagtggagatgtctgatatgtcattttcaaaggactggagcttctacattctcgctcacaccgagtttactccaaccgaaacagatacctacgcttgtcgggtgaaacatgcctccatggcagaaccaaagactgtgtactgggatagggacatg

SEQ ID NO: 223 - protein, 20 aa, synthetic construct
GGGGSGGGGSGGGGSGGGGS

SEQ ID NO: 224 - DNA, 60 bp, synthetic construct
  source 1..60: mol_type = other DNA
gggggcggcgggtccggggggggcggcagtggaggcggcggaagcggcggcggaggctca

SEQ ID NO: 225 - protein, 348 aa, synthetic construct; identical to SEQ ID NO: 9

SEQ ID NO: 226 - DNA, 1044 bp, synthetic construct
  source 1..1044: mol_type = other DNA
gggccccacagcctgcggtacttcgtgaccgcagtgagccgccccggactgggagagcctaggtacatggaggtgggatacgtggacgacaccgagttcgtcaggttcgactctgacgccgaaaaccctaggtacgagcccagagcccggtggatggaacaggagggaccagagtactgggaacgcgagacccagaaggcaaagggaaacgagcagtccttccgtgtggacctccgaacgctgctggggtactacaaccagtcaaagggcgggtctcatacaatccaggtgatcagcggatgtgaggtagggagcgacggcaggctgctgcggggctaccagcagtacgcctatgacggctgcgactacatcgctctgaacgaagatctcaagacctggacagccgcagacatggccgccctcatcactaagcataagtgggaacaggctggggaggccgagagactgagagcctatctggagggcacttgcgtggaatggctgagacgataccttaagaacggtaatgccacactgctgagaacagactccccaaaggcccacgtgacccaccactctagaccagaggacaaggtgacactcagatgctgggccctgggcttctaccctgctgacattacactcacctggcagctgaacggcgaggagctcatccaggacatggagctggtggagacaaggcccgcaggcgacggtaccttccagaagtgggcaagcgtggtggtccctctcgggaaggagcagtactacacatgccacgtgtatcatcaggggctgcctgagcccctgacactgaggtgggagcccccccccagcacagttagcaacatggctaccgtggctgtgctggtggtgctgggcgccgccatcgtgacaggggccgtggtcgctttcgtgatgaagatgcggcgaagaaatacaggcggcaagggcggcgactatgctctggctcctggatctcagaccagcgacctcagtctgcccgactgtaaggtgatggtgcacgacccacactccctggca

SEQ ID NO: 227 - protein, 21 aa, synthetic construct
GSGEGRGSLLTCGDVEENPGP

SEQ ID NO: 228 - DNA, 63 bp, synthetic construct
  source 1..63: mol_type = other DNA
ggcagtggcgaaggaaggggctccctgctgacctgcggggacgtggaggaaaaccccggacct

SEQ ID NO: 229 - protein, 116 aa, synthetic construct
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDF

SEQ ID NO: 230 - DNA, 348 bp, synthetic construct
  source 1..348: mol_type = other DNA
atgatggaccaggccagaagcgcgtttagcaatctgtttggtggcgaacctctgtcttacactagattcagtctggccaggcaggtggacggagacaactcccacgtggagatgaagctggctgtggacgaggaggagaacgccgacaacaacacaaaggccaacgtgaccaagcctaagaggtgcagcggttctatctgttacggcaccatcgctgtgatcgtgtttttcctgataggctttatgatcggctacctgggctactgcaagggcgtggagcctaagaccgaatgcgagcggctggccgggacagagtcccctgtgagggaggagcctggcgaggacttc

SEQ ID NO: 231 - protein, 5 aa, synthetic construct
GGGGS

SEQ ID NO: 232 - DNA, 15 bp, synthetic construct
  source 1..15: mol_type = other DNA
gggggcggcggcagt

SEQ ID NO: 233 - protein, 77 aa, synthetic construct
TTCPPPVSIEHADIRVKNYSVNSRERYVCNSGFKRKAGTSTLIECVINKNTNVAHWTTPSLKCIRDPSLAHYSPVPT

SEQ ID NO: 234 - DNA, 231 bp, synthetic construct
  source 1..231: mol_type = other DNA
acaacctgcccccctcctgtgtccattgagcatgccgacatcagggtgaaaaattattccgtgaactctagagagagatacgtgtgtaacagcggcttcaaaagaaaggccggcacatctaccctgatcgaatgtgtgatcaacaagaacacaaacgtggcacattggactacccccagcctgaaatgcatcagggacccctctctggcccactacagtcccgtgcctaca

SEQ ID NO: 235 - protein, 20 aa, synthetic construct
SGGSGGGGSGGGSGGGGSLQ

SEQ ID NO: 236 - DNA, 60 bp, synthetic construct
  source 1..60: mol_type = other DNA
agcggcgggtccggcggcggcggcagcggggggggtagcggcggcggcggctccctgcag

SEQ ID NO: 237 - protein, 114 aa, synthetic construct
NWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 238 - DNA, 342 bp, synthetic construct
  source 1..342: mol_type = other DNA
aattggatcgacgtgcgctacgatctggagaagatcgagtccttgattcagtccattcacatagacaccaccctgtatacagacagcgactttcacccatcttgtaaggtgaccgccatgaactgttttctgctggagctgcaggtgatcctgcatgaatacagcaacatgaccctgaacgagaccgtgcgcaacgtgctgtatctggccaactccaccctgagctctaataagaacgtggccgagagcgggtgtaaggagtgtgaggagctggaggagaagacctttacagagtttctgcagagcttcatccggattgtgcagatgtttattaacaccagc

SEQ ID NO: 239 - protein, 8 aa, synthetic construct
DYKDDDDK

SEQ ID NO: 240 - DNA, 24 bp, synthetic construct
  source 1..24: mol_type = other DNA
gactacaaggacgacgatgacaag

SEQ ID NO: 241 - protein, 22 aa, synthetic construct
GSGATNFSLLKQAGDVEENPGP

SEQ ID NO: 242 - DNA, 66 bp, synthetic construct
  source 1..66: mol_type = other DNA
ggatcaggcgccaccaatttttctctcctcaagcaagccggcgacgtggaggagaacccaggccct

SEQ ID NO: 243 - protein, 306 aa, synthetic construct; identical to SEQ ID NO: 19

SEQ ID NO: 244 - DNA, 918 bp, synthetic construct
  source 1..918: mol_type = other DNA
atggcctgcaattgtcaactgatgcaggatacccccctgctgaagtttccctgtccacgcctgatcctgctgttcgtgctcctgatcaggctgagtcaggtgtcttctgacgtggatgagcagctgagtaagagcgtcaaggataaggtgctgctcccttgcagatataatagcccccatgaggatgagagcgaggacaggatctactggcagaagcacgataaggtggtgctgtctgtgattgctggcaaactgaaggtgtggcctgagtataagaacagaaccctgtacgataacactacctacagcctgatcatcctgggcctggtgctgagcgacaggggcacatactcttgtgtggtgcagaagaaagagaggggaacctacgaggtgaaacatctggctctggtaaagctgtccatcaaggccgacttctccacaccaaatatcaccgagagcggaaacccctccgctgacacaaagcggatcacctgtttcgcttccggtggcttccccaaacccaggttcagctggctggagaacggaagagagctgccaggcatcaacaccaccatcagccaggaccccgagtctgagctgtacaccatcagctcccagctggacttcaacacaaccaggaaccacactatcaagtgcctgatcaaatatggcgacgcccacgtcagcgaagacttcacctgggagaagccccctgaggacccacccgattccaagaatacactggtgcttttcggcgccgggtttggtgccgtgattaccgtggtggtgattgtggtgattattaagtgcttctgtaagcacaggtcctgcttcagaaggaacgaggctagccgcgaaacaaacaacagtctgacgttcggccccgaagaggccctggccgagcagacagtgttcctc

SEQ ID NO: 245 - protein, 1199 aa, synthetic construct
MARSVTLVFLVLVSLTGLYASIINFEKLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAGSGEGRGSLLTCGDVEENPGPMMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFGGGGSTTCPPPVSIEHADIRVKNYSVNSRERYVCNSGFKRKAGTSTLIECVINKNTNVAHWTTPSLKCIRDPSLAHYSPVPTSGGSGGGGSGGGSGGGGSLQNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSDYKDDDDKGSGATNFSLLKQAGDVEENPGPMACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 246 - DNA, 3606 bp, synthetic construct
  source 1..3606: mol_type = other DNA
atggccaggtctgtgacactggtgtttctggtgctggtgtccctgacaggactctacgcctctatcatcaatttcgaaaaactggggggcggtgccagcgggggaggcggctccggcgggggcggctccatccagaagaccccccagatccaggtctactctcgacaccctcctgagaacggcaagccaaacatcctgaactgctatgtgacccagttccatcccccacacattgagatccagatgctgaagaacggcaagaagatcccaaaagtggagatgtctgatatgtcattttcaaaggactggagcttctacattctcgctcacaccgagtttactccaaccgaaacagatacctacgcttgtcgggtgaaacatgcctccatggcagaaccaaagactgtgtactgggatagggacatggggggcggcgggtccggggggggcggcagtggaggcggcggaagcggcggcggaggctcagggccccacagcctgcggtacttcgtgaccgcagtgagccgccccggactgggagagcctaggtacatggaggtgggatacgtggacgacaccgagttcgtcaggttcgactctgacgccgaaaaccctaggtacgagcccagagcccggtggatggaacaggagggaccagagtactgggaacgcgagacccagaaggcaaagggaaacgagcagtccttccgtgtggacctccgaacgctgctggggtactacaaccagtcaaagggcgggtctcatacaatccaggtgatcagcggatgtgaggtagggagcgacggcaggctgctgcggggctaccagcagtacgcctatgacggctgcgactacatcgctctgaacgaagatctcaagacctggacagccgcagacatggccgccctcatcactaagcataagtgggaacaggctggggaggccgagagactgagagcctatctggagggcacttgcgtggaatggctgagacgataccttaagaacggtaatgccacactgctgagaacagactccccaaaggcccacgtgacccaccactctagaccagaggacaaggtgacactcagatgctgggccctgggcttctaccctgctgacattacactcacctggcagctgaacggcgaggagctcatccaggacatggagctggtggagacaaggcccgcaggcgacggtaccttccagaagtgggcaagcgtggtggtccctctcgggaaggagcagtactacacatgccacgtgtatcatcaggggctgcctgagcccctgacactgaggtgggagcccccccccagcacagttagcaacatggctaccgtggctgtgctggtggtgctgggcgccgccatcgtgacaggggccgtggtcgctttcgtgatgaagatgcggcgaagaaatacaggcggcaagggcggcgactatgctctggctcctggatctcagaccagcgacctcagtctgcccgactgtaaggtgatggtgcacgacccacactccctggcaggcagtggcgaaggaaggggctccctgctgacctgcggggacgtggaggaaaaccccggacctatgatggaccaggccagaagcgcgtttagcaatctgtttggtggcgaacctctgtcttacactagattcagtctggccaggcaggtggacggagacaactcccacgtggagatgaagctggctgtggacgaggaggagaacgccgacaacaacacaaaggccaacgtgaccaagcctaagaggtgcagcggttctatctgttacggcaccatcgctgtgatcgtgtttttcctgataggctttatgatcggctacctgggctactgcaagggcgtggagcctaagaccgaatgcgagcggctggccgggacagagtcccctgtgagggaggagcctggcgaggacttcgggggcggcggcagtacaacctgcccccctcctgtgtccattgagcatgccgacatcagggtgaaaaattattccgtgaactctagagagagatacgtgtgtaacagcggcttcaaaagaaaggccggcacatctaccctgatcgaatgtgtgatcaacaagaacacaaacgtggcacattggactacccccagcctgaaatgcatcagggacccctctctggcccactacagtcccgtgcctacaagcggcgggtccggcggcggcggcagcggggggggtagcggcggcggcggctccctgcagaattggatcgacgtgcgctacgatctggagaagatcgagtccttgattcagtccattcacatagacaccaccctgtatacagacagcgactttcacccatcttgtaaggtgaccgccatgaactgttttctgctggagctgcaggtgatcctgcatgaatacagcaacatgaccctgaacgagaccgtgcgcaacgtgctgtatctggccaactccaccctgagctctaataagaacgtggccgagagcgggtgtaaggagtgtgaggagctggaggagaagacctttacagagtttctgcagagcttcatccggattgtgcagatgtttattaacaccagcgactacaaggacgacgatgacaagggatcaggcgccaccaatttttctctcctcaagcaagccggcgacgtggaggagaacccaggccctatggcctgcaattgtcaactgatgcaggatacccccctgctgaagtttccctgtccacgcctgatcctgctgttcgtgctcctgatcaggctgagtcaggtgtcttctgacgtggatgagcagctgagtaagagcgtcaaggataaggtgctgctcccttgcagatataatagcccccatgaggatgagagcgaggacaggatctactggcagaagcacgataaggtggtgctgtctgtgattgctggcaaactgaaggtgtggcctgagtataagaacagaaccctgtacgataacactacctacagcctgatcatcctgggcctggtgctgagcgacaggggcacatactcttgtgtggtgcagaagaaagagaggggaacctacgaggtgaaacatctggctctggtaaagctgtccatcaaggccgacttctccacaccaaatatcaccgagagcggaaacccctccgctgacacaaagcggatcacctgtttcgcttccggtggcttccccaaacccaggttcagctggctggagaacggaagagagctgccaggcatcaacaccaccatcagccaggaccccgagtctgagctgtacaccatcagctcccagctggacttcaacacaaccaggaaccacactatcaagtgcctgatcaaatatggcgacgcccacgtcagcgaagacttcacctgggagaagccccctgaggacccacccgattccaagaatacactggtgcttttcggcgccgggtttggtgccgtgattaccgtggtggtgattgtggtgattattaagtgcttctgtaagcacaggtcctgcttcagaaggaacgaggctagccgcgaaacaaacaacagtctgacgttcggccccgaagaggccctggccgagcagacagtgttcctctgatgatga

SEQ ID NO: 247 - protein, 27 aa, synthetic construct
MALQIPSLLLSAAVVVLMVLSSPGTEG

SEQ ID NO: 248 - DNA, 81 bp, synthetic construct
  source 1..81: mol_type = other DNA
atggctctgcagataccctccttgcttctgtccgctgccgtagtggtgctgatggtgcttagcagccctgggactgaaggt

SEQ ID NO: 249 - protein, 17 aa, synthetic construct
ISQAVHAAHAEINEAGR

SEQ ID NO: 250 - DNA, 51 bp, synthetic construct
  source 1..51: mol_type = other DNA
atatctcaagccgttcacgccgcccatgcggagataaatgaagctggtagg

SEQ ID NO: 251 - protein, 11 aa, synthetic construct
GGGGSGGGGSG

SEQ ID NO: 252 - DNA, 33 bp, synthetic construct
  source 1..33: mol_type = other DNA
ggaggcggaggctccggaggcggcgggagcgga

SEQ ID NO: 253 - protein, 238 aa, synthetic construct; identical to SEQ ID NO: 37

SEQ ID NO: 254 - DNA, 714 bp, synthetic construct
  source 1..714: mol_type = other DNA
ggcgacagtgagcgtcactttgtctatcagttcatgggcgagtgctactttaccaatggcacacaacggataagatatgtgacccggtacatttacaatcgagaagagtatgtgcgatatgactctgacgtcggcgagcatcgggccgtaaccgagctgggcagacccgatgcagagtattggaatagccagccggagatcctggaacgcactagggccgagctggacacagtttgccggcacaattacgagggtccagaaacacacacctcactgaggaggctggagcagcctaacgtcgttatctctctgagccgcaccgaggctctcaaccatcacaacacattggtttgctctgtgactgatttctacccagccaagatcaaggttcgctggtttaggaatgggcaagaggagactgtaggtgtctcaagtacacagctgatcagaaacggagattggacattccaggtactggttatgctggaaatgactcctcgcaggggagaggtgtacacatgccacgtcgaacatccatccctcaaatctcccatcactgtggaatggagagcccagagtgagtccgcatggagtaaaatgctgagcggaattggtggatgtgtgctcggggttattttcttggggctcgggttgttcattcggcatcgctcacagaaaggaccgcgagggccaccaccagcgggtctgctgcag

SEQ ID NO: 255 - protein, 22 aa, synthetic construct
GSGATNFSLLKQAGDVEENPGP

SEQ ID NO: 256 - DNA, 66 bp, synthetic construct
  source 1..66: mol_type = other DNA
ggcagcggcgccacaaacttctctctgctaaagcaagcaggtgatgttgaagaaaaccccgggcct

SEQ ID NO: 257 - protein, 256 aa, synthetic construct; identical to SEQ ID NO: 45

SEQ ID NO: 258 - DNA, 768 bp, synthetic construct
  source 1..768: mol_type = other DNA
atgcccagatccagagcactgatccttggtgttctggcccttacaacgatgcttagtttgtgtggcggggaggatgacatcgaagctgatcatgtgggcacgtacggtatctccgtgtaccagtctcccggggatattggacagtatacgttcgaatttgatggcgacgagctcttctacgtggacctcgataagaaggaaactgtctggatgttgcctgagtttgggcagctggcatcatttgaccctcaaggagggctgcagaatatcgcggtcgtgaagcacaatcttggcgtactgacaaagcgatccaacagtaccccggctactaacgaggcaccccaagcaaccgtgtttcctaagtccccagtgctgctcggccagcccaacaccctcatctgtttcgtagacaacatctttcctcccgtcattaacatcacatggctgcgtaactccaaatcagtggctgatggggtgtatgaaacctctttcttcgtgaacagggactacagttttcacaagctgtcttatctcacgttcatccctagcgacgatgacatttacgactgcaaagtggaacattggggcctggaagagcctgtcctgaagcactgggaaccggagattcccgcccctatgagcgagctgacagaaaccgttgtctgtgcactgggtctgtcagtcggcctcgtgggaattgtggtcggtaccatattcatcattcagggactgagaagcggaggcaccagccgtcaccccggaccactc

SEQ ID NO: 259 - protein, 21 aa, synthetic construct
GSGEGRGSLLTCGDVEENPGP

SEQ ID NO: 260 - DNA, 63 bp, synthetic construct; identical to SEQ ID NO: 194
  source 1..63: mol_type = other DNA

SEQ ID NO: 261 - protein, 335 aa, synthetic construct; identical to SEQ ID NO: 97

SEQ ID NO: 262 - DNA, 1005 bp, synthetic construct
  source 1..1005: mol_type = other DNA
atgtgtccacagaagctgaccatctcatggtttgccatagtgctgctggtgtccccactgatggccatgtgggagctggagaaggacgtgtacgtcgtggaggtggactggacccccgatgctccaggcgagacagtgaacctgacctgcgacacccccgaggaggacgatatcacctggacatccgatcagagacacggagtgatcggctccggcaagaccctgactattaccgtgaaggaatttctggacgctgggcagtacacttgtcacaaaggcggagagacactgtctcattctcacctgctgctgcataaaaaggagaacgggatctggagcactgaaatcctgaagaactttaagaacaagaccttcctgaagtgcgaggcccccaactacagcgggagattcacatgcagctggctggtgcagcgcaacatggatctgaagtttaacatcaagtccagcagttcatctccagacagcagggcagtgacatgcggcatggcttccctgtctgccgagaaggtgaccctggaccagcgcgattatgagaagtacagcgtgagctgccaggaggatgtgacctgtcctaccgccgaggagacactgccaatcgaactggccctggaagctagacagcagaacaagtacgagaattactccacctcctttttcatccgtgacattatcaagccggaccctccaaagaacctgcagatgaagccactgaaaaattcccaggtggaagtgagctgggagtaccccgattcctggagcacaccccactcctacttcagcctgaagttcttcgtgagaatccagaggaagaaggagaagatgaaagagactgaggagggctgtaaccagaaaggcgcattcctggtggagaaaacctctaccgaagtgcagtgcaaggggggcaatgtgtgtgtgcaggcccaggacaggtactataattcttcctgcagcaagtgggcatgcgtgccatgtagggtgagaagt

SEQ ID NO: 263 - protein, 15 aa, synthetic construct
GGGGSGGGGSGGGGS

SEQ ID NO: 264 - DNA, 45 bp, synthetic construct
  source 1..45: mol_type = other DNA
ggaggcggtggaagcggaggcggcgggtctggcgggggtggcagc

SEQ ID NO: 265 - protein, 193 aa, synthetic construct; identical to SEQ ID NO: 93

SEQ ID NO: 266 - DNA, 579 bp, synthetic construct
  source 1..579: mol_type = other DNA
cgggtgatccctgtgagcggcccagcacgctgtctgagtcagtctcggaacctgctgaaaaccaccgacgatatggtgaagaccgctagagaaaagctgaagcattacagctgtactgctgaggatattgaccacgaggatatcactcgggatcagactagcaccctgaagacatgtctgcccctggagctgcacaagaacgagtcttgtctggcaaccagggagaccagcagtaccacaaggggcagctgcctgcctccccagaagactagcctcatgatgacactgtgcctgggcagcatctatgaggatctcaagatgtaccagaccgagttccaggccatcaacgccgccctccagaatcacaatcaccagcagattatcctggacaagggcatgctggtggccattgacgagctcatgcagtctctgaatcacaacggcgagacactgagacagaagccacccgtcggggaggctgacccttatcgggttaaaatgaagctgtgtatcctgctgcacgccttttccactagagtggtgaccattaatcgcgtcatggggtacctgagctccgcc

SEQ ID NO: 267 - protein, 5 aa, synthetic construct
GGGGS

SEQ ID NO: 268 - DNA, 15 bp, synthetic construct
  source 1..15: mol_type = other DNA
ggcggcggcggcagc

SEQ ID NO: 269 - protein, 8 aa, synthetic construct
DYKDDDDK

SEQ ID NO: 270 - DNA, 24 bp, synthetic construct
  source 1..24: mol_type = other DNA
gactacaaggacgacgacgacaag

SEQ ID NO: 271 - protein, 117 aa, synthetic construct; identical to SEQ ID NO: 199

SEQ ID NO: 272 - DNA, 351 bp, synthetic construct
  source 1..351: mol_type = other DNA
gtgatctctaacagcgtgatgtacttcagcagcgtggtgcccgtgctgcagaaagtgaatagtacaaccacaaaacccgtgctgagaacccccagcccagtgcacccaacaggcacctctcagcctcaaagacctgaagactgccgccctaggggctccgtgaagggcaccggactggacttcgcttgcgatatctacatctgggcccccctggcaggcatctgcgtggccctgctgctgagcctgatcattaccctgatctgctatcacaggtccaggaagcgcgtgtgcaagtgccccaggccactggtgagacaggagggcaaacccaggccaagcgaaaagattgtc

SEQ ID NO: 273 - protein, 22 aa, synthetic construct
GSGATNFSLLKQAGDVEENPGP

SEQ ID NO: 274 - DNA, 66 bp, synthetic construct; identical to SEQ ID NO: 242
  source 1..66: mol_type = other DNA

SEQ ID NO: 275 - protein, 306 aa, synthetic construct; identical to SEQ ID NO: 19

SEQ ID NO: 276 - DNA, 927 bp, synthetic construct
  source 1..927: mol_type = other DNA
atggcctgcaattgtcaactgatgcaggatacccccctgctgaagtttccctgtccacgcctgatcctgctgttcgtgctcctgatcaggctgagtcaggtgtcttctgacgtggatgagcagctgagtaagagcgtcaaggataaggtgctgctcccttgcagatataatagcccccatgaggatgagagcgaggacaggatctactggcagaagcacgataaggtggtgctgtctgtgattgctggcaaactgaaggtgtggcctgagtataagaacagaaccctgtacgataacactacctacagcctgatcatcctgggcctggtgctgagcgacaggggcacatactcttgtgtggtgcagaagaaagagaggggaacctacgaggtgaaacatctggctctggtaaagctgtccatcaaggccgacttctccacaccaaatatcaccgagagcggaaacccctccgctgacacaaagcggatcacctgtttcgcttccggtggcttccccaaacccaggttcagctggctggagaacggaagagagctgccaggcatcaacaccaccatcagccaggaccccgagtctgagctgtacaccatcagctcccagctggacttcaacacaaccaggaaccacactatcaagtgcctgatcaaatatggcgacgcccacgtcagcgaagacttcacctgggagaagccccctgaggacccacccgattccaagaatacactggtgcttttcggcgccgggtttggtgccgtgattaccgtggtggtgattgtggtgattattaagtgcttctgtaagcacaggtcctgcttcagaaggaacgaggctagccgcgaaacaaacaacagtctgacgttcggccccgaagaggccctggccgagcagacagtgttcctctgatgatga

SEQ ID NO: 277 - protein, 1593 aa, synthetic construct
MALQIPSLLLSAAVVVLMVLSSPGTEGISQAVHAAHAEINEAGRGGGGSGGGGSGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGSGATNFSLLKQAGDVEENPGPMPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGSGEGRGSLLTCGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSDYKDDDDKVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIVGSGATNFSLLKQAGDVEENPGPMACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 278 - DNA, 4788 bp, synthetic construct
  source 1..4788: mol_type = other DNA
atggctctgcagataccctccttgcttctgtccgctgccgtagtggtgctgatggtgcttagcagccctgggactgaaggtatatctcaagccgttcacgccgcccatgcggagataaatgaagctggtaggggaggcggaggctccggaggcggcgggagcggaggcgacagtgagcgtcactttgtctatcagttcatgggcgagtgctactttaccaatggcacacaacggataagatatgtgacccggtacatttacaatcgagaagagtatgtgcgatatgactctgacgtcggcgagcatcgggccgtaaccgagctgggcagacccgatgcagagtattggaatagccagccggagatcctggaacgcactagggccgagctggacacagtttgccggcacaattacgagggtccagaaacacacacctcactgaggaggctggagcagcctaacgtcgttatctctctgagccgcaccgaggctctcaaccatcacaacacattggtttgctctgtgactgatttctacccagccaagatcaaggttcgctggtttaggaatgggcaagaggagactgtaggtgtctcaagtacacagctgatcagaaacggagattggacattccaggtactggttatgctggaaatgactcctcgcaggggagaggtgtacacatgccacgtcgaacatccatccctcaaatctcccatcactgtggaatggagagcccagagtgagtccgcatggagtaaaatgctgagcggaattggtggatgtgtgctcggggttattttcttggggctcgggttgttcattcggcatcgctcacagaaaggaccgcgagggccaccaccagcgggtctgctgcagggcagcggcgccacaaacttctctctgctaaagcaagcaggtgatgttgaagaaaaccccgggcctatgcccagatccagagcactgatccttggtgttctggcccttacaacgatgcttagtttgtgtggcggggaggatgacatcgaagctgatcatgtgggcacgtacggtatctccgtgtaccagtctcccggggatattggacagtatacgttcgaatttgatggcgacgagctcttctacgtggacctcgataagaaggaaactgtctggatgttgcctgagtttgggcagctggcatcatttgaccctcaaggagggctgcagaatatcgcggtcgtgaagcacaatcttggcgtactgacaaagcgatccaacagtaccccggctactaacgaggcaccccaagcaaccgtgtttcctaagtccccagtgctgctcggccagcccaacaccctcatctgtttcgtagacaacatctttcctcccgtcattaacatcacatggctgcgtaactccaaatcagtggctgatggggtgtatgaaacctctttcttcgtgaacagggactacagttttcacaagctgtcttatctcacgttcatccctagcgacgatgacatttacgactgcaaagtggaacattggggcctggaagagcctgtcctgaagcactgggaaccggagattcccgcccctatgagcgagctgacagaaaccgttgtctgtgcactgggtctgtcagtcggcctcgtgggaattgtggtcggtaccatattcatcattcagggactgagaagcggaggcaccagccgtcaccccggaccactcggctccggcgagggcaggggaagtcttctaacatgcggggacgtggaggaaaatcccggcccaatgtgtccacagaagctgaccatctcatggtttgccatagtgctgctggtgtccccactgatggccatgtgggagctggagaaggacgtgtacgtcgtggaggtggactggacccccgatgctccaggcgagacagtgaacctgacctgcgacacccccgaggaggacgatatcacctggacatccgatcagagacacggagtgatcggctccggcaagaccctgactattaccgtgaaggaatttctggacgctgggcagtacacttgtcacaaaggcggagagacactgtctcattctcacctgctgctgcataaaaaggagaacgggatctggagcactgaaatcctgaagaactttaagaacaagaccttcctgaagtgcgaggcccccaactacagcgggagattcacatgcagctggctggtgcagcgcaacatggatctgaagtttaacatcaagtccagcagttcatctccagacagcagggcagtgacatgcggcatggcttccctgtctgccgagaaggtgaccctggaccagcgcgattatgagaagtacagcgtgagctgccaggaggatgtgacctgtcctaccgccgaggagacactgccaatcgaactggccctggaagctagacagcagaacaagtacgagaattactccacctcctttttcatccgtgacattatcaagccggaccctccaaagaacctgcagatgaagccactgaaaaattcccaggtggaagtgagctgggagtaccccgattcctggagcacaccccactcctacttcagcctgaagttcttcgtgagaatccagaggaagaaggagaagatgaaagagactgaggagggctgtaaccagaaaggcgcattcctggtggagaaaacctctaccgaagtgcagtgcaaggggggcaatgtgtgtgtgcaggcccaggacaggtactataattcttcctgcagcaagtgggcatgcgtgccatgtagggtgagaagtggaggcggtggaagcggaggcggcgggtctggcgggggtggcagccgggtgatccctgtgagcggcccagcacgctgtctgagtcagtctcggaacctgctgaaaaccaccgacgatatggtgaagaccgctagagaaaagctgaagcattacagctgtactgctgaggatattgaccacgaggatatcactcgggatcagactagcaccctgaagacatgtctgcccctggagctgcacaagaacgagtcttgtctggcaaccagggagaccagcagtaccacaaggggcagctgcctgcctccccagaagactagcctcatgatgacactgtgcctgggcagcatctatgaggatctcaagatgtaccagaccgagttccaggccatcaacgccgccctccagaatcacaatcaccagcagattatcctggacaagggcatgctggtggccattgacgagctcatgcagtctctgaatcacaacggcgagacactgagacagaagccacccgtcggggaggctgacccttatcgggttaaaatgaagctgtgtatcctgctgcacgccttttccactagagtggtgaccattaatcgcgtcatggggtacctgagctccgccggcggcggcggcagcgactacaaggacgacgacgacaaggtgatctctaacagcgtgatgtacttcagcagcgtggtgcccgtgctgcagaaagtgaatagtacaaccacaaaacccgtgctgagaacccccagcccagtgcacccaacaggcacctctcagcctcaaagacctgaagactgccgccctaggggctccgtgaagggcaccggactggacttcgcttgcgatatctacatctgggcccccctggcaggcatctgcgtggccctgctgctgagcctgatcattaccctgatctgctatcacaggtccaggaagcgcgtgtgcaagtgccccaggccactggtgagacaggagggcaaacccaggccaagcgaaaagattgtcggatcaggcgccaccaatttttctctcctcaagcaagccggcgacgtggaggagaacccaggccctatggcctgcaattgtcaactgatgcaggatacccccctgctgaagtttccctgtccacgcctgatcctgctgttcgtgctcctgatcaggctgagtcaggtgtcttctgacgtggatgagcagctgagtaagagcgtcaaggataaggtgctgctcccttgcagatataatagcccccatgaggatgagagcgaggacaggatctactggcagaagcacgataaggtggtgctgtctgtgattgctggcaaactgaaggtgtggcctgagtataagaacagaaccctgtacgataacactacctacagcctgatcatcctgggcctggtgctgagcgacaggggcacatactcttgtgtggtgcagaagaaagagaggggaacctacgaggtgaaacatctggctctggtaaagctgtccatcaaggccgacttctccacaccaaatatcaccgagagcggaaacccctccgctgacacaaagcggatcacctgtttcgcttccggtggcttccccaaacccaggttcagctggctggagaacggaagagagctgccaggcatcaacaccaccatcagccaggaccccgagtctgagctgtacaccatcagctcccagctggacttcaacacaaccaggaaccacactatcaagtgcctgatcaaatatggcgacgcccacgtcagcgaagacttcacctgggagaagccccctgaggacccacccgattccaagaatacactggtgcttttcggcgccgggtttggtgccgtgattaccgtggtggtgattgtggtgattattaagtgcttctgtaagcacaggtcctgcttcagaaggaacgaggctagccgcgaaacaaacaacagtctgacgttcggccccgaagaggccctggccgagcagacagtgttcctctgatgatga

SEQ ID NO: 279 - protein, 8 aa, synthetic construct
STYVIPRL

SEQ ID NO: 280 - DNA, 24 bp, synthetic construct
  source 1..24: mol_type = other DNA
tcgacgtacgtcattcctcgcctt

SEQ ID NO: 281 - protein, 1150 aa, synthetic construct
MARSVTLVFLVLVSLTGLYASTYVIPRLGGGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAGSGEGRGSLLTCGDVEENPGPMYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIVGSGATNFSLLKQAGDVEENPGPMACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 282 - DNA, 3453 bp, synthetic construct
  source 1..3453: mol_type = other DNA
atggctcgctcggtgaccctggtctttctggtgcttgtctcactgaccggcctgtatgcttcgacgtacgtcattcctcgccttggcggaggtgcctctggcggtgggggcagcggtggagggggcagtatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggggggggaggctccggtggaggggggtctggagggggggggtctggtggaggcggaagtggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgggctccggcgagggcaggggaagtcttctaacatgcggggacgtggaggaaaatcccggcccaatgtactcaatgcagctggctagttgtgtgaccctgaccctcgtgctgctcgtgaacagcgccccaacctcaagctctacctccagtagcacagccgaagctcagcagcagcagcaacagcagcagcagcagcagcagcacctggagcagctgctgatggacctgcaggagctgctgagccggatggagaactacaggaacctgaagctgcctaggatgctgacattcaagttctacctgccaaagcaggccaccgagctgaaggacctgcagtgtctggaggacgagctgggccccctgcgccacgtgctcgacctgacacagtccaagtccttccagctggaggacgcagagaacttcatctccaacatcagagtgaccgtggtgaaactgaagggctctgacaacacctttgagtgtcagttcgacgacgagagtgccactgtggtggatttcctgaggcggtggatcgctttctgccagagcattatctctaccagcccacagggtggcggcggaagcgtcatcagcaacagcgtgatgtatttctcctctgtggtgcctgtgctgcagaaggtgaacagcaccaccaccaagcctgtgctgaggactccaagccccgtgcaccccactggtactagccagcctcagcgccccgaggactgtagacctagaggatctgtgaaaggaacaggcctggactttgcatgtgacatctatatttgggcaccacttgccggcatttgcgtggccctgctgctgtccctgatcatcacactgatctgctaccacaggagccgaaagcgtgtttgcaaatgtcccaggccgctagtcagacaggaaggcaagcccagaccttcagagaaaattgtgggttcaggggctaccaacttttccctccttaagcaggccggagatgtcgaggagaatcctggccctatggcgtgtaattgccagctcatgcaggacacacctctgttgaaattcccgtgtccaaggcttatcctgctgttcgtgctcttgatacgactcagccaggtgtcaagcgacgttgatgagcagctgtctaagagcgtgaaggataaggtgttgctgccctgtcggtacaatagcccacatgaggacgagtcagaggatcgcatctattggcagaaacacgacaaagtggtgctgagcgtgatcgctggcaagctgaaagtttggcccgagtacaagaaccggacactgtacgacaataccacatactccctgattattctggggctcgtgctctccgatagaggcacttatagctgcgttgtacagaagaaggaaagagggacttatgaggttaaacaccttgctctggtgaagctgagtatcaaggctgacttctccacgccaaatataaccgaatcaggaaatcctagtgccgatactaaacgcatcacttgttttgccagtggtgggttccccaaaccgagattctcttggctggaaaacggaagggagttgcccggcattaacaccaccatttctcaagaccccgaatccgagctttacaccattagcagccaacttgacttcaacacaacacggaaccacaccatcaagtgtctgatcaagtatggcgacgcacatgtcagtgaggatttcacatgggagaaaccacccgaagatcctccagactccaagaacactctcgtgctgtttggtgcaggatttggagccgtaataaccgtagtggtcattgtcgtcatcatcaagtgcttctgcaaacaccgttcttgctttcgacgcaatgaagcctctagggaaacaaacaactctctgacttttggccctgaagaagccctggcagagcaaacggtctttctgtag

SEQ ID NO: 283 - protein, 8 aa, synthetic construct
KILTFDRL

SEQ ID NO: 284 - DNA, 24 bp, synthetic construct
  source 1..24: mol_type = other DNA
aagatcctgaccttcgacaggctc

(19) United States
(12) Patent Application Publication
(10) Pub. No.: US 2024/0360198 A1
(43) Pub. Date: Oct. 31, 2024

(54) IMMUNOREGULATORY METHOD, NUCLEIC ACID COMPOSITION FOR IMMUNOREGULATION, AND USE THEREOF

(71) Applicants: NATIONAL UNIVERSITY CORPORATION KANAZAWA UNIVERSITY, Kanazawa (JP); NISSAN CHEMICAL CORPORATION, Tokyo (JP)

(72) Inventors: Rikinari HANAYAMA, Kanazawa (JP); Tomoyoshi YAMANO, Kanazawa (JP); Kazutaka MATOBA, Shiraoka (JP)

(73) Assignees: NATIONAL UNIVERSITY CORPORATION KANAZAWA UNIVERSITY, Kanazawa (JP); NISSAN CHEMICAL CORPORATION, Tokyo (JP)

(21) Appl. No.: 18/687,485

(22) PCT Filed: Sep. 1, 2022
(86) PCT No.: PCT/JP2022/033026

§ 371 Date: Feb. 28, 2024

(30) Foreign Application Priority Data
Sep. 1, 2021 (JP) 2021-142688

(51) Int. Cl. C07K 14/74; A61K 38/00; A61P 35/00; C07K 14/495; C07K 14/54; C07K 14/55; C07K 16/28

(52) U.S. Cl. CPC C07K 14/70539; A61P 35/00; C07K 14/495; C07K 14/5406; C07K 14/5412; C07K 14/5434; C07K 14/5443; C07K 14/55; C07K 16/28; A61K 38/00; C07K 2319/03

(57) ABSTRACT

Provided is a polynucleotide comprising at least one sequence encoding a fusion protein comprising (a) an antigen-presenting MHC molecule, which can be presented outside the membrane of a cell or an extracellular vesicle; (b) at least one T-cell stimulatory cytokine or subunit thereof, which can be presented outside the membrane of a cell or an extracellular vesicle; (c) a T-cell costimulatory molecule, which can be presented outside the membrane of a cell or an extracellular vesicle; (d) an antigen-presenting MHC molecule and at least one T-cell stimulatory cytokine or subunit thereof, which can be presented outside the membrane of a cell or an extracellular vesicle; or (e) an antigen-presenting MHC molecule, at least one T-cell stimulatory cytokine p or subunit thereof, and a T-cell costimulatory molecule, which can be presented outside the membrane of a cell or an extracellular vesicle.

### CROSS REFERENCE

This application claims priority from Japanese Patent Application 2021-142688 filed on Sep. 1, 2021, the entire contents of which, including prior art documents, are incorporated herein by reference.

### TECHNICAL FIELD

The present invention relates to an immunoregulatory method, a nucleic acid composition for immunoregulation, and a use thereof.

### BACKGROUND ART

It is known that antigen-specific T cells (for example, cytotoxic T cells, helper T cells, and the like) play a central role in an immune reaction such as elimination of cancer cells and the like by living bodies or regulation of responses to auto-antigens, allergic substances, and the like. The antigen-specific T cells recognize a binding complex of MHC molecules on cell surfaces of antigen-presenting cells such as dendritic cells or macrophages, and antigens derived from cancer, allergic substances, and the like, at a T cell receptor, and activate, proliferate, and differentiate. The activated antigen-specific T cells specifically injure cancer cells and the like presenting antigens, and regulate responses to auto-antigens, allergic substances, and the like. Therefore, it is considered that activation, proliferation, and differentiation of the antigen-specific T cells are particularly important in the immune reaction.

As a method for activating the antigen-specific T cells, not only a method for expressing a chimeric antigen receptor in T cells that has already been put into practical use, but also other methods have been developed. For example, Patent Literature 1 discloses that nanoparticles containing MHC molecules and T-cell costimulatory molecules on surfaces thereof proliferate antigen-specific T cells. In addition, Non Patent Literature 1 discloses that exosomes in which IL-12 is expressed on membranes by PTGFRN proliferate model antigen-specific CD8-positive T cells.

### CITATION LIST

### Patent Literature

Patent Literature 1: JP 2016-520518 A

### Non Patent Literatures

Non Patent Literature 1: Katherine Kirwin, et al., “Exosome Surface Display of IL-12 Results in Tumor-Retained Pharmacology with Superior Potency and Limited Systemic Exposure Compared to Recombinant IL-12”, Nov. 6, 2019, 34th Annual Meeting of the Society for Immuno-therapy of Cancer
Non Patent Literature 2: Journal of Extracellular Vesicles (2018): 7:1535750
Non Patent Literature 3: ONCOIMMUNOLOGY 2020, VOL. 9, NO. 1, e1673125

### SUMMARY OF INVENTION

### Technical Problem

As a novel method capable of activating antigen-specific T cells, the present inventors tried a method using extracellular vesicles containing MHC molecules and T-cell costimulatory molecules in membranes. However, when an attempt was made to active antigen-specific T cells using the extracellular vesicles, it was found for the first time that antigen-specific T cells could not be satisfactorily activated.

Therefore, an object of the present invention is to provide a novel immunoregulatory method, a nucleic acid composition for immunoregulation, and a use thereof.

### Solution to Problem

In view of the above problems, as a result of conducting intensive studies, the present inventors have surprisingly found that T cells can be activated using polynucleotides capable of producing cells or extracellular vesicles containing MHC molecules and T-cell stimulatory cytokines in membranes, thereby completing the present invention.

Therefore, the present invention includes the followings.
[0] A cell or an extracellular vesicle presenting an antigen-presenting MHC molecule and a T-cell stimulatory cytokine outside membrane thereof.
[1] An antigen-presenting cell or an antigen-presenting extracellular vesicle, the membrane of which contains:
   (A) a protein which comprises an antigen-presenting MHC molecule and is capable of presenting the antigen outside the membrane; and
   (B) a protein which comprises a T-cell stimulatory cytokine or a subunit thereof and is capable of presenting the T-cell stimulatory cytokine outside the membrane.
[2] The antigen-presenting cell or the antigen-presenting extracellular vesicle according to [1], wherein
   the protein (A) which comprises an antigen-presenting MHC molecule and is capable of presenting the antigen outside the membrane is a fusion protein or a protein complex which comprises an antigen-presenting MHC molecule, and a membrane protein capable of being expressed in membrane of a cell or an extracellular vesicle or a transmembrane domain thereof or a protein capable of binding to membrane of a cell or an extracellular vesicle or a membrane-binding domain thereof, and is capable of presenting the antigen outside the membrane.
[3] The antigen-presenting cell or the antigen-presenting extracellular vesicle according to [1], wherein
   the protein (B) which comprises a T-cell stimulatory cytokine or a subunit thereof and is capable of presenting the T-cell stimulatory cytokine outside the membrane is a fusion protein which comprises a T-cell stimulatory cytokine or a subunit thereof, and a membrane protein capable of being expressed in membrane of a cell or an extracellular vesicle or a transmembrane domain thereof or a protein capable of binding to membrane of a cell or an extracellular vesicle or a membrane-binding domain thereof, and is capable of presenting the T-cell stimulatory cytokine outside the membrane.
[4] The antigen-presenting extracellular vesicle according to [1], wherein the protein (A) which comprises an antigen-presenting MHC molecule and is capable of presenting the antigen outside the membrane is:
   1) a fusion protein or a protein complex which comprises an antigen-presenting MHC molecule and a Tetraspanin or a transmembrane domain thereof or MFG-E8 or a membrane-binding domain thereof, and is capable of presenting the antigen outside the membrane:
   2) a fusion protein which comprises an amino acid sequence containing, from an N-terminal side thereof,
   (A-1) an amino acid sequence of an MHC molecule-restricted antigen peptide,
   (A-2) a spacer sequence which may be optionally present,
   (A-3) an amino acid sequence of a single chain MHC molecule,
   (A-4) a spacer sequence which may be optionally present, and
   (A-5) an amino acid sequence of a Tetraspanin or a membrane-binding domain thereof, in this order;
   3) a protein complex which contains
   a fusion protein which comprises an amino acid sequence containing, from an N-terminal side thereof,
   (A-1) an amino acid sequence of an MHC molecule-restricted antigen peptide,
   (A-2) a spacer sequence which may be optionally present,
   (A-3) an amino acid sequence of an MHC class Iα chain, β₂ microglobulin, an MHC class IIα chain, or an MHC class IIβ chain,
   (A-4) a spacer sequence which may be optionally present, and
   (A-5) an amino acid sequence of a Tetraspanin or a transmembrane domain thereof, in this order, and
   a protein which comprises
   (A-6) an amino acid sequence of β₂ microglobulin, an MHC class Iα chain, an MHC class IIβ chain, or an MHC class IIα chain;
   4) a fusion protein which comprises an amino acid sequence containing, from an N-terminal side thereof,
   (A-1) an MHC class I molecule-restricted antigen peptide,
   (A-2) an amino acid sequence of a spacer sequence which may be optionally present,
   (A-3) an amino acid sequence of a single chain MHC class I molecule,
   (A-4) a spacer sequence which may be optionally present, and
   (A-5) an amino acid sequence of a Tetraspanin or a transmembrane domain thereof, in this order;
   5) a protein complex which contains
   a fusion protein which comprises an amino acid sequence containing, from an N-terminal side thereof,
   (A-1) an amino acid sequence of an MHC class II molecule-restricted antigen peptide,
   (A-2) a spacer sequence which may be optionally present,
   (A-3) an amino acid sequence of an MHC class IIβ chain,
   (A-4) a spacer sequence which may be optionally present, and
   (A-5) an amino acid sequence of a Tetraspanin or a transmembrane domain thereof, in this order, and a protein which comprises
   (A-6) an amino acid sequence of an MHC class IIα chain; or
   6) a protein complex which contains
   a fusion protein which comprises an amino acid sequence containing, from an N-terminal side thereof,
   (A-1) an amino acid sequence of an MHC class II molecule-restricted antigen peptide,
   (A-2) a spacer sequence which may be optionally present,
   (A-3) an amino acid sequence of an MHC class IIβ chain,
   (A-4) a spacer sequence which may be optionally present, and
   (A-5) an amino acid sequence of a Tetraspanin or a transmembrane domain thereof, in this order, and a protein which comprises
   (A-6) an amino acid sequence of an MHC class IIα chain.
[5] The antigen-presenting extracellular vesicle according to [1], wherein the protein (B) which comprises a T-cell stimulatory cytokine or a subunit thereof and is capable of presenting the T-cell stimulatory cytokine outside the membrane is:
   1) a fusion protein which comprises a T-cell stimulatory cytokine or a subunit thereof and a partial sequence of a Tetraspanin, and is capable of presenting the T-cell stimulatory cytokine outside the membrane, in which the partial sequence of the Tetraspanin contains at least two transmembrane domains, and the T-cell stimulatory cytokine is disposed between the two transmembrane domains:
   2) a fusion protein which comprises a T-cell stimulatory cytokine or a subunit thereof and MFG-E8 or a domain thereof, and is capable of presenting the T-cell stimulatory cytokine outside the membrane:
   3) a fusion protein which comprises an amino acid sequence containing, from an N-terminal side thereof,
   (B-1) a partial sequence of a Tetraspanin containing, from an N-terminal side thereof, a transmembrane domain 1, a small extracellular loop, a transmembrane domain 2, a small intracellular loop, and a transmembrane domain 3,
   (B-2) a spacer sequence which may be optionally present,
   (B-3) an amino acid sequence of a T-cell stimulatory cytokine,
   (B-4) a spacer sequence which may be optionally present, and
   (B-5) a partial sequence of a Tetraspanin containing a transmembrane domain 4, in this order,
   the fusion protein being capable of presenting the T-cell stimulatory cytokine outside the membrane; or
   4) a fusion protein which comprises an amino acid sequence containing, from an N-terminal side thereof,
   (B-3) an amino acid sequence of a T-cell stimulatory cytokine,
   (B-4) a spacer sequence which may be optionally present, and
   (B-5) an amino acid sequence of MFG-E8 or a membrane-binding domain thereof, in the order,
   the fusion protein being capable of presenting the first T-cell stimulatory cytokine outside the membrane.
[6] The antigen-presenting cell or the antigen-presenting extracellular vesicle according to [1], wherein the T-cell stimulatory cytokine or subunit thereof is IL-2, IL-4, IL-6, IL-12, a subunit of IL-12, IL-15, or TGF-β.
[7] The antigen-presenting cell or the antigen-presenting extracellular vesicle according to [1], wherein
   the membrane further contains (C) a protein which comprises a T-cell costimulatory molecule and is capable of allowing the T-cell costimulatory molecule to interact with T cells.
[8] The antigen-presenting cell or the antigen-presenting extracellular vesicle according to [7], wherein
   the protein (C) which comprises a T-cell costimulatory molecule and is capable of allowing the T-cell costimulatory molecule to interact with T cells is a fusion protein which comprises a T-cell costimulatory molecule, and a membrane protein capable of being expressed in membrane of a cell or an extracellular vesicle or a transmembrane domain thereof or a protein capable of binding to a membrane of a cell or an extracellular vesicle or a domain thereof, and is capable of allowing the T-cell costimulatory molecule to interact with T cells.
[9] The antigen-presenting extracellular vesicle according to [7], wherein
   the protein (C) which comprises a T-cell costimulatory molecule and is capable of allowing the T-cell costimulatory molecule to interact with T cells contains:
   1) a fusion protein which comprises a T-cell costimulatory molecule and a Tetraspanin or a transmembrane domain thereof or MFG-E8 or a domain thereof, and is capable of allowing the T-cell costimulatory molecule to interact with T cells; and
   2) a fusion protein which comprises an amino acid sequence containing, from an N-terminal side thereof,
   (C-1) an amino acid sequence of a T-cell costimulatory molecule,
   (C-2) a spacer sequence which may be optionally present, and
   (C-3) an amino acid sequence of a Tetraspanin or a transmembrane domain thereof,
   the fusion protein being capable of allowing the T-cell costimulatory molecule to interact with T cells.
[10] The antigen-presenting cell according to [7], wherein
   the protein (C) which comprises a T-cell costimulatory molecule and is capable of allowing the T-cell costimulatory molecule to interact with T cells is a fusion protein which comprises a T-cell costimulatory molecule containing a transmembrane domain, and is capable of allowing the T-cell costimulatory molecule to interact with T cells.
[1I] The antigen-presenting extracellular vesicle according to [1], wherein the extracellular vesicle is an exosome.
[1A]
   An antigen-presenting cell or an antigen-presenting extracellular vesicle, the membrane of which contains,
   (D) a fusion protein which comprises an antigen-presenting MHC molecule and a T-cell stimulatory cytokine or a subunit thereof, and is capable of presenting the antigen and the T-cell stimulatory cytokine outside the membrane.
[2A] The antigen-presenting cell or the antigen-presenting extracellular vesicle according to [1A], wherein the fusion protein (D) which comprises an antigen-presenting MHC molecule and a T-cell stimulatory cytokine or a subunit thereof, and is capable of presenting the antigen and the T-cell stimulatory cytokine outside the membrane comprises the antigen-presenting MHC molecule, the T-cell stimulatory cytokine or subunit thereof, and a membrane protein capable of being localized to membrane of a cell or an extracellular vesicle or a transmembrane domain thereof or a protein capable of binding to membrane of a cell or an extracellular vesicle or a membrane-binding domain thereof.
[3A] The antigen-presenting extracellular vesicle according to [2A], wherein the membrane protein capable of being localized to membrane of an extracellular vesicle or the protein capable of binding to membrane of an extracellular vesicle is a Tetraspanin or MFG-E8.
[4A] The antigen-presenting cell according to [2A], wherein the membrane protein capable of being localized to membrane of a cell or the protein capable of binding to membrane of a cell is CD8.
[5A] The antigen-presenting extracellular vesicle according to [3A], wherein the fusion protein (D) comprises:
   1) an amino acid sequence encoding, from an N-terminal side thereof,
   (D-1) an MHC molecule-restricted antigen peptide,
   (D-2) a spacer sequence which may be optionally present,
   (D-3) a single chain MHC molecule,
   (D-4) a spacer sequence which may be optionally present, and
   (D-5) a fusion peptide comprising a Tetraspanin or a transmembrane domain thereof or MFG-E8 or a transmembrane domain thereof, and the T-cell stimulatory cytokine or subunit thereof, in this order;
   2) an amino acid sequence encoding, from an N-terminal side thereof,
   (D-1) a fusion peptide comprising a Tetraspanin or a transmembrane domain thereof or MFG-E8 or a transmembrane domain thereof, and the T-cell stimulatory cytokine or subunit thereof,
   (D-2) a spacer sequence which may be optionally present,
   (D-3) a single chain MHC molecule,
   (D-4) a spacer sequence which may be optionally present, and
   (D-5) an MHC molecule-restricted antigen peptide, in this order; or
   3) an amino acid sequence encoding, from an N-terminal side thereof,
   (1) the at least one T-cell stimulatory cytokine or subunit thereof,
   (2) a spacer sequence which may be optionally present, and
   (3) MFG-E8, in this order.
[6A] The antigen-presenting cell according to [4A], wherein the fusion protein (D) comprises:
   1) an amino acid sequence encoding, from an N-terminal side thereof,
   (D-1) an MHC molecule-restricted antigen peptide,
   (D-2) a spacer sequence which may be optionally present,
   (D-3) a single chain MHC molecule containing a transmembrane domain,
   (D-4) a spacer sequence which may be optionally present, and
   (D-5) a fusion peptide comprising CD8 or a transmembrane domain thereof and the T-cell stimulatory cytokine or subunit thereof, in this order; or
   2) an amino acid sequence encoding, from an N-terminal side thereof,
   (D-1) a fusion peptide comprising CD8 or a transmembrane domain thereof and the T-cell stimulatory cytokine or subunit thereof,
   (D-2) a spacer sequence which may be optionally present,
   (D-3) a single chain MHC molecule containing a transmembrane domain,
   (D-4) a spacer sequence which may be optionally present, and
   (D-5) an MHC molecule-restricted antigen peptide, in this order.
[8A] The antigen-presenting cell according to [6A], wherein the fusion peptide comprises an amino acid sequence encoding, from an N-terminal side thereof,
   (1) the T-cell stimulatory cytokine or subunit thereof,
   (2) a spacer sequence which may be optionally present, and
   (3) CD8 or a transmembrane domain thereof, in this order.
[9A]
   The antigen-presenting extracellular vesicle according to [5A], wherein the MHC molecule-restricted antigen peptide is an MHC class I molecule-restricted antigen peptide, and the single chain MHC molecule contains an extracellular domain of an MHC class Iα chain.
[10A]
   The antigen-presenting extracellular vesicle according to [5A], wherein the MHC molecule-restricted antigen peptide is an MHC class II molecule-restricted antigen peptide, and the single chain MHC molecule contains an extracellular domain of an MHC class IIα chain and/or an extracellular domain of an MHC class IIβ chain.
[11A]
   The antigen-presenting cell according to [7A], wherein the MHC molecule-restricted antigen peptide is an MHC class I molecule-restricted antigen peptide, and the single chain MHC molecule containing a transmembrane domain comprises an MHC class Iα chain.
[12A]
   The antigen-presenting cell according to [7A], wherein the MHC molecule-restricted antigen peptide is an MHC class II molecule-restricted antigen peptide, and the single chain MHC molecule containing a transmembrane domain comprises an MHC class IIα chain and/or an MHC class IIβ chain.
[13A]
   The antigen-presenting cell or the antigen-presenting extracellular vesicle according to [1A], wherein the membrane of the antigen-presenting cell or the antigen-presenting extracellular vesicle further contains (C) a protein which comprises a T-cell costimulatory molecule and is capable of allowing the T-cell costimulatory molecule to interact with T cells.
[14A]
   The antigen-presenting cell or the antigen-presenting extracellular vesicle according to [13A], wherein the protein (C) comprises the T-cell costimulatory molecule, and a membrane protein capable of being expressed in membrane of a cell or an extracellular vesicle or a transmembrane domain thereof or a protein capable of binding to membrane of a cell or an extracellular vesicle or a domain thereof.
[15A]
   The antigen-presenting extracellular vesicle according to [14A], wherein the protein (C) comprises the T-cell costimulatory molecule, and a Tetraspanin or a transmembrane domain thereof or MFG-E8 or a domain thereof.
[16A]
   The antigen-presenting cell according to [14A], wherein the protein (C) comprises a T-cell costimulatory molecule containing a transmembrane domain.
[17A] The antigen-presenting extracellular vesicle according to [1A], wherein the extracellular vesicle is an exosome.
[1B]
   The antigen-presenting cell or the antigen-presenting extracellular vesicle according to [1], wherein the protein (A) and the protein (B) are fused to each other to constitute one protein.
[2B]
   The antigen-presenting cell or the antigen-presenting extracellular vesicle according to [8], wherein the protein (A) and the protein (C) are fused to each other to constitute one protein.
[3B]
   The antigen-presenting cell or the antigen-presenting extracellular vesicle according to [8], wherein the protein (B) and the protein (C) are fused to each other to constitute one protein.
[4B]
   The antigen-presenting cell or the antigen-presenting extracellular vesicle according to [8], wherein the protein (A), the protein (B), and the protein (C) are fused to each other to constitute one protein.
[5B]
   The antigen-presenting cell or the antigen-presenting extracellular vesicle according to [13A], wherein the protein (D) and the protein (C) are fused to each other to constitute one protein.
[6B] The antigen-presenting cell or the antigen-presenting extracellular vesicle according to any one of [1B] to [5B], wherein the extracellular vesicle is an exosome.
[7B] A pharmaceutical composition comprising the antigen-presenting cell or the antigen-presenting extracellular vesicle according to any one of [1] to [6B] and a pharmacologically acceptable carrier.
[1C] A pharmaceutical composition for treating or preventing cancer comprising the antigen-presenting cell or the antigen-presenting extracellular vesicle according to any one of [1] to [6B], wherein the antigen peptide preferably includes a cancer antigen peptide.
[2C] A pharmaceutical composition for treating or preventing an autoimmune disease comprising the antigen-presenting cell or the antigen-presenting extracellular vesicle according to any one of [1] to [6B]: wherein the antigen peptide preferably includes an auto-antigen peptide.
[3C] A pharmaceutical composition for treating or preventing an allergic disease comprising the antigen-presenting cell or the antigen-presenting extracellular vesicle according to any one of [1] to [6B]: wherein the antigen peptide preferably includes an allergen.
[4C] The pharmaceutical composition according to [1C], further containing an immune checkpoint inhibitor.
[5C] The pharmaceutical composition according to [4C], wherein the immune checkpoint inhibitor is present on the membrane of the antigen-presenting cell or the antigen-presenting extracellular vesicle.
[6C] The pharmaceutical composition according to [4C] or [5C], wherein the immune checkpoint inhibitor is selected from the group consisting of an anti-PD-1 antibody or an active fragment thereof, an anti-CTLA-4 antibody or an active fragment thereof, and a PD-L1 antibody or an active fragment thereof.
[7C] A pharmaceutical composition for treating or preventing an infectious disease comprising the antigen-presenting cell or the antigen-presenting extracellular vesicle according to any one of [1] to [6B] and a pharmacologically acceptable carrier: wherein the antigen peptide is preferably derived from an infectious pathogen that causes the infectious disease.
[1D] The antigen-presenting cell or the antigen-presenting extracellular vesicle according to any one of [1] to [6B] for use in treating or preventing cancer: wherein the antigen peptide preferably includes a cancer antigen peptide.
[2D] The antigen-presenting cell or the antigen-presenting extracellular vesicle according to any one of [1] to [6B] for use in treating or preventing an autoimmune disease: wherein the antigen peptide preferably includes an auto-antigen peptide.
[3D] The antigen-presenting cell or the antigen-presenting extracellular vesicle according to any one of [1] to [6B] for use in treating or preventing an allergic disease; wherein the antigen peptide preferably includes an allergen.
[4D] The antigen-presenting cell or the antigen-presenting extracellular vesicle according to [1D] used together with an immune checkpoint inhibitor.
[5D] The antigen-presenting cell or the antigen-presenting extracellular vesicle according to [4D], wherein the immune checkpoint inhibitor is present on the membrane.
[6D] The antigen-presenting cell or the antigen-presenting extracellular vesicle for use according to [4D] or [5D], wherein the immune checkpoint inhibitor is selected from the group consisting of an anti-PD-1 antibody or an active fragment thereof, an anti-CTLA-4 antibody or an active fragment thereof, and a PD-L1 antibody or an active fragment thereof.
[7D] The antigen-presenting cell or the antigen-presenting extracellular vesicle according to any one of [1] to [6B] for use in treating or preventing an infectious disease: wherein the antigen peptide is preferably derived from an infectious pathogen that causes the infectious disease.
[1E] Use of the antigen-presenting cell or the antigen-presenting extracellular vesicle according to any one of [1] to [6B] in the manufacture of a medicament for treating or preventing cancer: wherein the antigen peptide preferably includes a cancer antigen peptide.
[2E] Use of the antigen-presenting cell or the antigen-presenting extracellular vesicle according to any one of [1] to [6B] in the manufacture of a medicament for treating or preventing an autoimmune disease: wherein the antigen peptide preferably includes an auto-antigen peptide.
[3E] Use of the antigen-presenting cell or the antigen-presenting extracellular vesicle according to any one of [1] to [6B] in the manufacture of a medicament for treating or preventing an allergic disease: wherein the antigen peptide preferably includes an allergen.
[4E] The use according to [1E], wherein the pharmaceutical is used together with an immune checkpoint inhibitor.
[5E] The use according to [4E], wherein the immune checkpoint inhibitor is present on the membrane of the antigen-presenting cell or the antigen-presenting extracellular vesicle.
[6E] The use according to [4E] or [5E], wherein the immune checkpoint inhibitor is selected from the group consisting of an anti-PD-1 antibody or an active fragment thereof, an anti-CTLA-4 antibody or an active fragment thereof, and a PD-L1 antibody or an active fragment thereof.
[7E] Use of the antigen-presenting cell or the antigen-presenting extracellular vesicle according to any one of [1] to [6B] in the manufacture of a medicament for treating or preventing an infectious disease: wherein the antigen peptide is preferably derived from an infectious pathogen that causes the infectious disease.
[1F] A method for treating or preventing cancer in a subject, the method comprising:
   administering an effective amount of the antigen-presenting cell or the antigen-presenting extracellular vesicle according to any one of [1] to [6B] to the subject to activate and/or proliferate T cells that recognize a caner antigen in the subject and to allow the activated and/or proliferated T cells to attack cancer cells: wherein the activated and/or proliferated T cells are preferably CD8-positive cytotoxic T cells, and the antigen peptide preferably includes a cancer antigen peptide.
[2F] A method for treating or preventing an autoimmune disease in a subject, the method comprising: administering an effective amount of the antigen-presenting cell or the antigen-presenting extracellular vesicle according to any one of [1] to [6B] to the subject to activate and/or proliferate T cells that recognize an auto-antigen in the subject and to desensitize an immune response to the auto-antigen in the subject, wherein the activated and/or proliferated T cells are preferably CD4-positive regulatory T cells (Treg), and the antigen peptide preferably includes an auto-antigen peptide.
[3F] A method for treating or preventing an allergic disease in a subject, wherein a method for treating or preventing an allergic disease comprises administering an effective amount of the antigen-presenting cell or the antigen-presenting extracellular vesicle according to any one of [1] to [6B] to the subject to activate and/or proliferate T cells that recognize an allergen in the subject and to desensitize an immune response to the auto-antigen in the subject: wherein the activated and/or proliferated T cells are preferably CD4-positive regulatory T cells (Treg), and the antigen peptide preferably includes an allergen.
[4F] The method according to [1F], wherein the antigen-presenting cell or the antigen-presenting extracellular vesicle is administered together with an immune checkpoint inhibitor.
[5F] The method according to [4F], wherein the immune checkpoint inhibitor is present on the membrane of the antigen-presenting cell or the antigen-presenting extracellular vesicle.
[6F] The method according to [4F] or [5F], wherein the immune checkpoint inhibitor is selected from the group consisting of an anti-PD-1 antibody or an active fragment thereof, an anti-CTLA-4 antibody or an active fragment thereof, and a PD-L1 antibody or an active fragment thereof.
[7F] A method for treating or preventing an infectious disease in a subject, the method comprising:
   administering an effective amount of the antigen-presenting cell or the antigen-presenting extracellular vesicle according to any one of [1] to [6B] to the subject to,
   1) secrete inflammatory cytokines and to activate innate immunity of the subject, and/or
   2) provide acquired immunity to an infectious pathogen that causes the infectious disease to the subject,
   so that, in the subject's body, the infectious pathogen that causes the infectious disease is eliminated and/or a proliferation of the infectious pathogen is suppressed.
[1G] A method for activating and/or proliferating T cells against a specific antigen, the method comprising contacting an effective amount of the antigen-presenting cell or the antigen-presenting extracellular vesicle according to any one of [1] to [6B] with T cells in vitro or ex vivo.
[1H] A fusion protein (A) comprising an antigen-presenting MHC molecule and capable of presenting the antigen-presenting MHC molecule outside membrane of a cell or an extracellular vesicle.
[2H] A fusion protein (B) comprising at least one T-cell stimulatory cytokine or a subunit thereof and capable of presenting the T-cell stimulatory cytokine outside membrane of a cell or an extracellular vesicle.
[3H] A fusion protein (C) comprising a T-cell costimulatory molecule and capable of presenting the T-cell costimulatory molecule outside membrane of a cell or an extracellular vesicle.
[4H] A fusion protein (D) comprising an antigen-presenting MHC molecule and at least one T-cell stimulatory cytokine or a subunit thereof, and capable of presenting the antigen and the T-cell stimulatory cytokine outside membrane of a cell or an extracellular vesicle.
[5H] A fusion protein (E) comprising an antigen-presenting MHC molecule, at least one T-cell stimulatory cytokine or a subunit thereof, and a T-cell costimulatory molecule, and capable of presenting the antigen, the T-cell stimulatory cytokine, and the T-cell costimulatory molecule outside membrane of a cell or an extracellular vesicle.
[1I] A polynucleotide comprising at least one sequence selected from the group consisting of:
   (a) a sequence encoding a fusion protein (A) which comprises an antigen-presenting MHC molecule and is capable of presenting the antigen-presenting MHC molecule outside membrane of a cell or an extracellular vesicle:
   (b) a sequence encoding a fusion protein (B) which comprises at least one T-cell stimulatory cytokine or subunit thereof and is capable of presenting the T-cell stimulatory cytokine outside membrane of a cell or an extracellular vesicle:
   (c) a sequence encoding a fusion protein (C) which comprises a T-cell costimulatory molecule and is capable of presenting the T-cell costimulatory molecule outside membrane of a cell or an extracellular vesicle:
   (d) a sequence encoding a fusion protein (D) which comprises an antigen-presenting MHC molecule and at least one T-cell stimulatory cytokine or subunit thereof and is capable of presenting the antigen and the T-cell stimulatory cytokine outside membrane of a cell or an extracellular vesicle; and
   (e) a sequence encoding a fusion protein (E) which comprises an antigen-presenting MHC molecule, at least one T-cell stimulatory cytokine or subunit thereof, and a T-cell costimulatory molecule, and is capable of presenting the antigen, the T-cell stimulatory cytokine, and the T-cell costimulatory molecule outside membrane of a cell or an extracellular vesicle.
[2I]
   The polynucleotide according to [1I], wherein the fusion protein defined as (A) comprises an antigen-presenting MHC molecule, and a membrane protein capable of being expressed in membrane of a cell or an extracellular vesicle or a transmembrane domain thereof or a protein capable of binding to membrane of a cell or an extracellular vesicle or a membrane-binding domain thereof.
[3I] The polynucleotide according to [1I], wherein the fusion protein defined as (A) comprises an antigen-presenting MHC molecule, and a Tetraspanin or a transmembrane domain thereof or MFG-E8 or a membrane-binding domain thereof.
[4I] The polynucleotide according to [1I], wherein the fusion protein defined as (A) comprises an antigen-presenting MHC molecule containing a cell transmembrane domain.
[5I] The polynucleotide according to [1I], wherein the fusion protein defined as (A) comprises an amino acid sequence containing, from an N-terminal side thereof,
   (A-1) an amino acid sequence of an MHC molecule-restricted antigen peptide,
   (A-2) a spacer sequence which may be optionally present,
   (A-3) an amino acid sequence of a single chain MHC molecule,
   (A-4) a spacer sequence which may be optionally present, and
   (A-5) an amino acid sequence of a Tetraspanin or a transmembrane domain thereof, in this order.
[6I] The polynucleotide according to [1I], wherein the fusion protein defined as (A) comprises an amino acid sequence containing, from an N-terminal side thereof,
   (A-1) an amino acid sequence of an MHC molecule-restricted antigen peptide,
   (A-2) a spacer sequence which may be optionally present,
   (A-3) an amino acid sequence of an MHC class Iα chain, β₂ microglobulin, an MHC class IIα chain, or an MHC class IIβ chain,
   (A-4) a spacer sequence which may be optionally present, and
   (A-5) an amino acid sequence of a Tetraspanin or a transmembrane domain thereof, in this order.
[7I] The polynucleotide according to [6I], wherein (A-6) an amino acid sequence of β₂ microglobulin, an MHC class Iα chain, an MHC class IIβ chain, or an MHC class IIα chain is further comprised.
[8I] The polynucleotide according to [1I], wherein the fusion protein defined as (A) comprises an amino acid sequence containing, from an N-terminal side thereof,
   (A-1) an amino acid sequence of an MHC class I molecule-restricted antigen peptide,
   (A-2) a spacer sequence which may be optionally present,
   (A-3) an amino acid sequence of a single chain MHC class I molecule,
   (A-4) a spacer sequence which may be optionally present, and
   (A-5) an amino acid sequence of a Tetraspanin or a transmembrane domain thereof, in this order.
[9I] The polynucleotide according to [1I], wherein the fusion protein defined as (A) comprises an amino acid sequence containing, from an N-terminal side thereof,
   (A-1) an amino acid sequence of an MHC class II molecule-restricted antigen peptide,
   (A-2) a spacer sequence which may be optionally present,
   (A-3) an amino acid sequence of an MHC class IIβ chain,
   (A-4) a spacer sequence which may be optionally present, and
   (A-5) an amino acid sequence of a Tetraspanin or a transmembrane domain thereof, in this order.
[10I] The polynucleotide according to [9I], wherein (A-6) a sequence encoding an amino acid sequence of an MHC class IIα chain is further comprised.
[11I] The polynucleotide according to [1I], wherein the fusion protein defined as (A) comprises an amino acid sequence containing, from an N-terminal side thereof,
   (A-1) an amino acid sequence of an MHC molecule-restricted antigen peptide,
   (A-2) a spacer sequence which may be optionally present, and
   (A-3) an amino acid sequence of a single chain MHC molecule containing a transmembrane domain, in this order.
[12I] The polynucleotide according to [1I], wherein the fusion protein defined as (A) comprises an amino acid sequence containing, from an N-terminal side thereof,
   (A-1) an amino acid sequence of an MHC molecule-restricted antigen peptide,
   (A-2) a spacer sequence which may be optionally present, and
   (A-3) an amino acid sequence of an MHC class Iα chain, a fusion protein of β₂ microglobulin and an MHC class Iα chain, an MHC class IIα chain, or an MHC class IIβ chain, in this order.
[13I] The polynucleotide according to [12I], wherein (A-6) a sequence encoding an amino acid sequence of β₂ microglobulin, an MHC class IIβ chain, or an MHC class IIα chain is further comprised.
[14I] The polynucleotide according to [1I], wherein the fusion protein defined as (A) comprises an amino acid sequence containing, from an N-terminal side thereof,
   (A-1) an amino acid sequence of an MHC class I molecule-restricted antigen peptide,
   (A-2) a spacer sequence which may be optionally present, and
   (A-3) an amino acid sequence of a single chain MHC class I molecule containing a transmembrane domain, in this order.
[15I] The polynucleotide according to [1I], wherein the fusion protein defined as (A) comprises an amino acid sequence containing, from an N-terminal side thereof,
   (A-1) an amino acid sequence of an MHC class II molecule-restricted antigen peptide,
   (A-2) a spacer sequence which may be optionally present, and
   (A-3) an amino acid sequence of an MHC class IIβ chain, in this order.
[16I] The polynucleotide according to [15I], wherein a sequence encoding an amino acid sequence of an MHC class IIα chain is further comprised.
[17I] The polynucleotide according to [1I], wherein the fusion protein defined as (B) comprises at least one T-cell stimulatory cytokine or subunit thereof, and a membrane protein capable of being expressed in membrane of a cell or an extracellular vesicle or a transmembrane domain thereof or a protein capable of binding to membrane of a cell or an extracellular vesicle or a domain thereof.
[18I] The polynucleotide according to [1I], in which the fusion protein defined as (B) comprises at least one T-cell stimulatory cytokine or subunit thereof and a partial sequence of a Tetraspanin, the partial sequence of the Tetraspanin contains at least two transmembrane domains, and the at least one T-cell stimulatory cytokine is disposed between the two transmembrane domains.
[19I] The polynucleotide according to [1I], wherein the fusion protein defined as (B) comprises at least one T-cell stimulatory cytokine or subunit thereof and MFG-E8 or a membrane-binding domain thereof.
[20I] The polynucleotide according to [1I], wherein the fusion protein defined as (B) comprises an amino acid sequence containing, from an N-terminal side thereof,
   (B-1) a partial sequence of a Tetraspanin containing, from an N-terminal side thereof, a transmembrane domain 1, a small extracellular loop, a transmembrane domain 2, a small intracellular loop, and a transmembrane domain 3,
   (B-2) a spacer sequence which may be optionally present,
   (B-3) an amino acid sequence of a first T-cell stimulatory cytokine,
   (B-4) a spacer sequence which may be optionally present, and
   (B-5) a partial sequence of a Tetraspanin containing a transmembrane domain 4, in this order.
[21I] The polynucleotide according to [1I], wherein the fusion protein defined as (B) comprises an amino acid sequence containing, from an N-terminal side thereof,
   (B-3) an amino acid sequence of a first T-cell stimulatory cytokine,
   (B-4) a spacer sequence which may be optionally present, and
   (B-5) an amino acid sequence of MFG-E8 or a membrane-binding domain thereof, in this order.
[22I] The polynucleotide according to [1I], wherein the fusion protein defined as (B) comprises at least one T-cell stimulatory cytokine or subunit thereof and CD8 or a transmembrane domain thereof.
[23I] The polynucleotide according to [1I], wherein the fusion protein defined as (B) comprises an amino acid sequence containing, from an N-terminal side thereof,
   (B-3) an amino acid sequence of a first T-cell stimulatory cytokine,
   (B-4) a spacer sequence which may be optionally present, and
   (B-5) an amino acid sequence of CD8 or a transmembrane domain thereof, in this order.
[24I] The polynucleotide according to [1I], wherein the T-cell stimulatory cytokine is IL-2, IL-4, IL-6, IL-12, a subunit of IL-12, IL-15, or TGF-β.
[25I] The polynucleotide according to [1I], wherein the fusion protein defined as (C) comprises a T-cell costimulatory molecule, and
   a membrane protein capable of being expressed in membrane of a cell or an extracellular vesicle or a transmembrane domain thereof, or
   a protein capable of binding to membrane of a cell or an extracellular vesicle or a membrane-binding domain thereof.
[26I] The polynucleotide according to [1I], wherein the fusion protein defined as (C) comprises a T-cell costimulatory molecule, and
   a Tetraspanin or a transmembrane domain thereof, or
   MFG-E8 or a membrane-binding domain thereof.
[27I] The polynucleotide according to [1I], wherein the fusion protein defined as (C) comprises a T-cell costimulatory molecule containing a transmembrane domain.
[28I] The polynucleotide according to [1I], wherein the fusion protein defined as (C) comprises an amino acid sequence containing, from an N-terminal side thereof,
   (C-1) an amino acid sequence of a T-cell costimulatory molecule,
   (C-2) a spacer sequence which may be optionally present, and
   (C-3) an amino acid sequence of a Tetraspanin or a transmembrane domain thereof, in this order.
[29I] The polynucleotide according to [1I], wherein the fusion protein defined as (D) comprises the antigen-presenting MHC molecule,
   the at least one T-cell stimulatory cytokine or subunit thereof, and
   a membrane protein capable of being localized to membrane of a cell or an extracellular vesicle or a transmembrane domain thereof, or
   a protein capable of binding to membrane of a cell or an extracellular vesicle or a membrane-binding domain thereof.
[30I] The polynucleotide according to [29I], wherein the membrane protein capable of being localized to membrane of an extracellular vesicle or the protein capable of binding to membrane of an extracellular vesicle is a Tetraspanin or MFG-E8.
[31I] The polynucleotide according to [29I], wherein the membrane protein capable of being localized to membrane of a cell or the protein capable of binding to membrane of a cell is CD8.
[32I] The polynucleotide according to [1I], wherein the fusion protein defined as (D) comprises an amino acid sequence containing, from an N-terminal side thereof,
   (D-1) an amino acid sequence of an MHC molecule-restricted antigen peptide,
   (D-2) a spacer sequence which may be optionally present,
   (D-3) an amino acid sequence of a single chain MHC molecule,
   (D-4) a spacer sequence which may be optionally present, and
   (D-5) an amino acid sequence of a fusion peptide comprising a Tetraspanin or a transmembrane domain thereof or MFG-E8 or a membrane-binding domain thereof, and the at least one T-cell stimulatory cytokine or subunit thereof, in this order.
[33I] The polynucleotide according to [1I], wherein the fusion protein defined as (D) comprises an amino acid sequence containing, from an N-terminal side thereof,
   (D-1) an amino acid sequence of a fusion peptide comprising a Tetraspanin or a transmembrane domain thereof or MFG-E8 or a membrane-binding domain thereof, and the at least one T-cell stimulatory cytokine or subunit thereof,
   (D-2) a spacer sequence which may be optionally present,
   (D-3) an amino acid sequence of a single chain MHC molecule,
   (D-4) a spacer sequence which may be optionally present, and
   (D-5) an amino acid sequence of an MHC molecule-restricted antigen peptide, in this order.
[34I] The polynucleotide according to [32I] or [33I], wherein the fusion peptide comprising a Tetraspanin or a transmembrane domain thereof or MFG-E8 or a membrane-binding domain thereof, and the at least one T-cell stimulatory cytokine or subunit thereof comprises an amino acid sequence containing, from an N-terminal side thereof,
   (1) a partial sequence of a Tetraspanin containing a transmembrane domain 1, a small extracellular loop, a transmembrane domain 2, a small intracellular loop, and a transmembrane domain 3,
   (2) a spacer sequence which may be optionally present,
   (3) an amino acid sequence of the at least one T-cell stimulatory cytokine or subunit thereof,
   (4) a spacer sequence which may be optionally present, and
   (5) a partial sequence of a Tetraspanin containing a transmembrane domain 4, in this order.
[35I] The polynucleotide according to [32I] or [33I], wherein the fusion peptide comprising a Tetraspanin or a transmembrane domain thereof or MFG-E8 or a membrane-binding domain thereof, and the at least one T-cell stimulatory cytokine or subunit thereof comprises an amino acid sequence containing, from an N-terminal side thereof,
   (1) an amino acid sequence of the at least one T-cell stimulatory cytokine or subunit thereof,
   (2) a spacer sequence which may be optionally present, and
   (3) an amino acid sequence of MFG-E8 or a membrane-binding domain thereof, in this order.
[36I] The polynucleotide according to [1I], wherein the fusion protein defined as (D) comprises the antigen-presenting MHC molecule, the at least one T-cell stimulatory cytokine or subunit thereof, and a membrane protein capable of being localized to membrane of a cell or a transmembrane domain thereof or a protein capable of binding to membrane of a cell or a membrane-binding domain thereof.
[37I] The polynucleotide according to [32I] or [33I], wherein the MHC molecule-restricted antigen peptide is an MHC class I molecule-restricted antigen peptide, and the single chain MHC molecule contains an extracellular domain of an MHC class Iα chain.
[38I] The polynucleotide according to [32I] or [33I], wherein the MHC molecule-restricted antigen peptide is an MHC class II molecule-restricted antigen peptide, and the single chain MHC molecule contains an extracellular domain of an MHC class IIα chain and/or an extracellular domain of an MHC class IIβ chain.
[1I] The polynucleotide according to [1I], comprising the sequence defined as (a) and the sequence defined as (b).
[2I] The polynucleotide according to [1J], wherein the polynucleotide encodes an amino acid sequence in which the fusion protein (A) and the fusion protein (B) are fused.
[3J] The polynucleotide according to [2j], wherein the polynucleotide encodes an amino acid sequence in which the fusion protein (A) and the fusion protein (B) are fused through at least one 2A peptide.
[4J] The polynucleotide according to [1J], further comprising the sequence defined as (c)
[5J] The polynucleotide according to [4J], wherein the polynucleotide encodes an amino acid sequence in which the fusion protein (A), the fusion protein (B), and the fusion protein (C) are fused.
[6J] The polynucleotide according to [5J], wherein the polynucleotide encodes an amino acid sequence in which the fusion protein (A), the fusion protein (B), and the fusion protein (C) are each fused through at least one independent 2A peptide.
[7J] The polynucleotide according to [6J], wherein the polynucleotide comprises, from a 5′ end:
   the sequence defined as (a);
   a sequence encoding at least one first 2A peptide:
   the sequence defined as (b);
   a sequence encoding at least one second 2A peptide; and
   the sequence defined as (c), in this order.
[8J] The polynucleotide according to [1I], comprising the sequence defined as (d).
[9J] The polynucleotide according to [8J], further comprising the sequence defined as (c).
[10J] The polynucleotide according to [9J], wherein the polynucleotide encodes an amino acid sequence in which the fusion protein (D) and the fusion protein (C) are fused.
[11J] The polynucleotide according to [10J], wherein the polynucleotide encodes an amino acid sequence in which the fusion protein (D) and the fusion protein (C) are fused through at least one 2A peptide.
[12J] The polynucleotide according to [1I], comprising the sequence defined as (e).
[13J] A vector comprising the polynucleotide according to any one of [1I] to [I2J].
[14J] A pharmaceutical composition comprising the polynucleotide according to any one of [1I] to [12J] or the vector according to [13J], and a pharmacologically acceptable carrier.
[1K] A cell transformed with the polynucleotide according to any one of [1I] to [12J] or the vector according to [13J].
[2K] A culture supernatant after culturing the cell according to [1K].
[3K] An antigen-presenting extracellular vesicle obtained from the culture supernatant according to [2K].
[4K] A method for preparing the antigen-presenting extracellular vesicle according to [1], the method comprising a step of collecting a culture supernatant obtained by culturing the cell according to [1K].
[5K] A pharmaceutical composition comprising the culture supernatant according to [2K].
[1L] A pharmaceutical composition for treating or preventing cancer, comprising the polynucleotide according to any one of [1I] to [12J] or the vector according to [13J].
[2L] A pharmaceutical composition for treating or preventing an autoimmune disease, comprising the polynucleotide according to any one of [1I] to [12J] or the vector according to [13J]: wherein the antigen peptide preferably includes an auto-antigen peptide.
[3L] A pharmaceutical composition for treating or preventing an allergic disease, comprising the polynucleotide according to any one of [1I] to [12J] or the vector according to [13J]: wherein the antigen peptide preferably includes an allergen.
[4L] A pharmaceutical composition for treating or preventing an infectious disease, comprising the polynucleotide according to any one of [1I] to [12J] or the vector according to [13J], and a pharmacologically acceptable carrier: wherein the antigen peptide is preferably derived from an infectious pathogen that causes an infectious disease.
[1M] The polynucleotide according to any one of [1I] to [12J] or the vector according to [13J] for use in treating or preventing cancer: wherein the antigen peptide preferably includes a cancer antigen peptide.
[2M] The polynucleotide according to any one of [1I] to [12J] or the vector according to [13J] for use in treating or preventing an autoimmune disease: wherein the antigen peptide preferably includes an auto-antigen peptide.
[3M] The polynucleotide according to any one of [1I] to [12J] or the vector according to [13J] for use in treating or preventing an allergic disease: wherein the antigen peptide preferably includes an allergen.
[4M] The polynucleotide according to any one of [1I] to [12J] or the vector according to [13J] for use in treating or preventing an infectious disease: wherein the antigen peptide is preferably derived from an infectious pathogen that causes the infectious disease.
[1N] Use of the polynucleotide according to any one of [1I] to [12J] or the vector according to [13J] in the manufacture of a medicament for treating or preventing cancer: wherein the antigen peptide preferably includes a cancer antigen peptide.
[2N] Use of the polynucleotide according to any one of [1I] to [12J] or the vector according to [13J] in the manufacture of a medicament for treating or preventing an autoimmune disease: wherein the antigen peptide preferably includes an auto-antigen peptide.
[3N] Use of the polynucleotide according to any one of [1I] to [12J] or the vector according to [13J] in the manufacture of a medicament for treating or preventing an allergic disease: wherein the antigen peptide preferably includes an allergen.
[4N] Use of the polynucleotide according to any one of [1I] to [12J] or the vector according to [13J] in the manufacture of a medicament for treating or preventing an infectious disease: wherein the antigen peptide is preferably derived from an infectious pathogen that causes the infectious disease.
[1O] A method for treating or preventing cancer in a subject, the method comprising
   administering an effective amount of the polynucleotide according to any one of [1I] to [12J] or the vector according to [13J] to the subject to activate and/or proliferate T cells that recognize a cancer antigen in the subject and to attack cancer cells in the activated and/or proliferated T cells: wherein the activated and/or proliferated T cells are preferably CD8-positive cytotoxic T cells, and the antigen peptide preferably includes a cancer antigen peptide.
[2O] A method for treating or preventing an autoimmune disease in a subject, the method comprising administering an effective amount of the polynucleotide according to any one of [1I] to [12J] or the vector according to [13J] to the subject to activate and/or proliferate T cells that recognize an auto-antigen in the subject and to desensitize an immune response to the auto-antigen in the subject: wherein the activated and/or proliferated T cells are preferably CD4-positive regulatory T cells (Treg), and the antigen peptide preferably includes an auto-antigen peptide.
[3O] A method for treating or preventing an allergic disease in a subject, wherein a method for treating or preventing an allergic disease comprises administering an effective amount of the polynucleotide according to any one of [1I] to [12J] or the vector according to [13J] to the subject to activate and/or proliferate T cells that recognize an allergen in the subject and to desensitize an immune response to the auto-antigen in the subject: wherein the activated and/or proliferated T cells are preferably CD4-positive regulatory T cells (Treg), and the antigen peptide preferably includes an allergen.
[4O] A method for treating or preventing an infectious disease in a subject, the method comprising:
   administering an effective amount of the polynucleotide according to any one of [1I] to [12J] or the vector according to [13J] to the subject to,
   1) secrete inflammatory cytokines and to activate innate immunity of the subject, and/or
   2) provide acquired immunity to an infectious pathogen that causes the infectious disease to the subject,
   so that, in the subject's body, the infectious pathogen that causes the infectious disease is eliminated and/or a proliferation of the infectious pathogen is suppressed.
[1P] A method for activating and/or proliferating T cells against a specific antigen, the method comprising: introducing the polynucleotide according to any one of [1I] to [12J] or the vector according to [13J] to cells in vitro or ex vivo to produce antigen-presenting cells and/or antigen-presenting extracellular vesicles; and contacting the produced antigen-presenting cells and/or antigen-presenting extracellular vesicles with T cells in vitro or ex vivo.

### Advantageous Effects of Invention

According to the present invention, it is possible to activate T cells by using a cell (antigen-presenting cell) containing an MHC molecule presenting an antigen and a T-cell stimulatory cytokine in membrane and a polynucleotide for producing an extracellular vesicle (antigen-presenting extracellular vesicle).

### BRIEF DESCRIPTION OF DRAWINGS

FIG. 1A illustrates a model diagram of an antigen peptide-single chain MHC class I molecule (sc-Trimer)-CD81 fusion protein.

FIG. 1B illustrates an amino acid sequence of the antigen peptide-single chain MHC class I molecule (sc-Trimer)-CD81 fusion protein.

FIG. 1C illustrates a model diagram of a CD80-CD9 fusion protein.

FIG. 1D illustrates an amino acid sequence of the CD80-CD9 fusion protein.

FIG. 1E illustrates a model diagram of a CD63-IL-2 fusion protein.

FIG. 1F illustrates an amino acid sequence of the CD63-IL-2 fusion protein.

FIG. 1G illustrates a model diagram of an antigen peptide-MHC class IIβ chain (sc-Dimer)-CD81 fusion protein.

FIG. 1H illustrates an amino acid sequence of the antigen peptide-MHC class IIβ chain (sc-Dimer)-CD81 fusion protein.

FIG. 1I illustrates an amino acid sequence of an MHC class IIα chain.

FIG. 1J illustrates a model diagram of a TGF-β-MFG-E8 fusion protein.

FIG. 1K illustrates an amino acid sequence of the TGF-β-MFG-E8 fusion protein.

FIG. 1L illustrates a model diagram of a CD81-IL-4 fusion protein.

FIG. 1M illustrates an amino acid sequence of the CD81-IL-4 fusion protein.

FIG. 1N illustrates a nucleic acid sequence of a sc-Trimer-CD81-IL-2 fusion protein.

FIG. 1O illustrates a nucleic acid sequence of the CD63-AkaLuc fusion protein.

FIG. 1P illustrates a nucleic acid sequence to code sc-Trimer-T2A-IL-2-CD8-P2A-CD80.

FIG. 2A illustrates a model diagram of an antigen-presenting extracellular vesicle of Example 1.

FIG. 2B illustrates a model diagram of an antigen-presenting extracellular vesicle of Example 2.

FIG. 2C illustrates a model diagram of an antigen-presenting extracellular vesicle of Example 3.

FIG. 2D illustrates a model diagram of an antigen-presenting extracellular vesicle of Example 4.

FIG. 2E illustrates a model diagram of an antigen-presenting extracellular vesicle of Example 5.

FIG. 2F illustrates a model diagram of an antigen-presenting extracellular vesicle of Example 6.

FIG. 2G illustrates a model diagram of an antigen-presenting extracellular vesicle of Example 7.

FIG. 2H illustrates a model diagram of an antigen-presenting extracellular vesicle of Example 8.

FIG. 2I illustrates a model diagram of an antigen-presenting extracellular vesicle of Example 9.

FIG. 2J illustrates a model diagram of an antigen-presenting extracellular vesicle of Example 11.

FIG. 2K illustrates a model diagram of antigen-presenting extracellular vesicles of other embodiments.

FIG. 3A illustrates results obtained by analyzing fusion proteins contained in the membrane of the antigen-presenting extracellular vesicle of Example 2 by flow cytometry in Test Example 1-1.

FIG. 3B illustrates results obtained by analyzing fusion proteins contained in the membrane of the antigen-presenting extracellular vesicle of Example 3 by flow cytometry in Test Example 1-2.

FIG. 3C illustrates results obtained by analyzing fusion proteins contained in the membrane of the antigen-presenting extracellular vesicle of Example 4 by flow cytometry in Test Example 1-3.

FIG. 3D illustrates results obtained by analyzing fusion proteins contained in the membrane of the antigen-presenting extracellular vesicle of Example 5 by flow cytometry in Test Example 1-4.

FIG. 3E illustrates results obtained by analyzing fusion proteins contained in the membrane of the antigen-presenting extracellular vesicle of Example 6 by flow cytometry in Test Example 1-5.

FIG. 3F illustrates results obtained by analyzing fusion proteins contained in the membrane of the antigen-presenting extracellular vesicle of Example 7 by flow cytometry in Test Example 1-6.

FIG. 3G illustrates results obtained by analyzing fusion proteins contained in the membrane of the antigen-presenting extracellular vesicle of Example 8 by flow cytometry in Test Example 1-7.

FIG. 3H illustrates results obtained by analyzing fusion proteins contained in the membrane of the antigen-presenting extracellular vesicle of Example 9 by flow cytometry in Test Example 1-8.

FIG. 4 illustrates results obtained by evaluating whether the antigen-presenting extracellular vesicles of Examples 1 and 2 activate antigen-specific CD8-positive T cells (OT-1 T cells) in vitro in Test Example 2.

FIG. 5 illustrates results obtained by evaluating whether the antigen-presenting extracellular vesicle of Example 2 activates antigen-specific CD8-positive T cells (OT-1) in vivo in Test Example 3.

FIG. 6 illustrates results obtained by evaluating whether the antigen-presenting extracellular vesicle of Example 3 activates antigen-specific CD4-positive T cells in vitro in Test Example 4.

FIG. 7 illustrates results obtained by evaluating whether the antigen-presenting extracellular vesicle of Example 4 induces differentiation of antigen-specific CD4-positive T cells (OT-2 T cells) into regulatory T cells in vitro in Test Example 5.

FIG. 8 illustrates results obtained by evaluating whether the antigen-presenting extracellular vesicles of Examples 3 and 5 induce differentiation of antigen-specific CD4-positive T cells (OT-2 T cells) into Th2T cells in vitro in Test Example 6.

FIG. 9 illustrates results obtained by evaluating whether the antigen-presenting extracellular vesicle of Example 6 induces differentiation of antigen-specific CD4-positive T cells into Th1 cells in vitro in Test Example 7.

FIG. 10 illustrates results obtained by evaluating whether the antigen-presenting extracellular vesicle of Example 7 induces differentiation of antigen-specific CD4-positive T cells into Th17 cells in vitro in Test Example 8.

FIG. 11 illustrates that antigen-specific CD8-positive T cells are remarkably proliferated by the antigen-presenting extracellular vesicles of Examples 1 and 8 in Test Example 9.

FIG. 12 illustrates that B16 melanoma cells are remarkably suppressed by the antigen-presenting extracellular vesicle of Example 8 in Test Example 10.

FIG. 13 illustrates results obtained by evaluating whether mRNA of Example 10 activates antigen-specific CD8-positive T cells (OT-1) in vivo in Test Example 11.

FIG. 14 illustrates results obtained by evaluating whether mRNA of Example 10 activates intrinsic antigen-specific CD8-positive T cells in vivo in Test Example 12.

FIG. 15 illustrates results obtained by evaluating whether the extracellular vesicle of Example 6 differentiates antigen-specific CD4-positive T cells into Th1 cells in vivo in Test Example 13.

FIG. 16 illustrates results obtained by evaluating whether the antigen-presenting extracellular vesicle of Example 6 inhibits proliferation of melanoma cells in vivo in Test Example 14.

FIG. 17 illustrates results of flow cytometry of the antigen-presenting extracellular vesicle of Example 11 in Test Example 15.

FIG. 18 illustrates results obtained by evaluating whether the antigen-presenting extracellular vesicle of Example 11 differentiates antigen-specific CD4-positive T cells into Th1 cells in vitro in Test Example 16.

FIG. 19 illustrates results obtained by evaluating whether the antigen-presenting extracellular vesicle of Example 12 inhibits proliferation of T-lymphoma cells in vivo in Test Example 17.

FIG. 20 illustrates results obtained by evaluating whether an antigen-MHC I complex, CD80, and IL-2 are expressed on cells by mRNA of Example 1A in vitro in Test Example 1A.

FIG. 21 illustrates results obtained by evaluating whether the antigen-presenting cells induced by mRNA of Example 1A proliferate antigen-specific CD8-positive T cells in vitro in Test Example 2A.

FIG. 22 illustrates results obtained by evaluating whether an antigen-MHC I complex, CD80, and IL-2 are expressed on cells by mRNA of Example 1A in vivo in Test Example 3A.

FIG. 23 illustrates results obtained by evaluating whether intrinsic OVA-reactive CD8T cells proliferate by mRNA of Example 1A in vivo in Test Example 4A.

FIG. 24 illustrates (a) a nucleic acid sequence of a sc-Trimer-T2A-IL-15sa-P2A-CD80 fusion protein, (b) a nucleic acid sequence of a sc-Trimer-T2A-IL-2-CD8-P2A-CD80 fusion protein presenting a neoantigen, (c) a nucleic acid sequence of an OVAp-MHCIIβ-P2A-MHCIIα-T2A-IL-12sc-CD8-P2A-CD80 fusion protein presenting, and (d) a nucleic acid sequence of a sc-Trimer-CD81-IL-2 fusion protein presenting a neoantigen.

FIG. 25 illustrates results obtained by evaluating whether an antigen-MHC I complex, CD80, and IL-15sa are expressed on cells by mRNA of Example 2A in vivo in Test Example 5A.

FIG. 26 illustrates results obtained by evaluating whether intrinsic OVA-reactive CD8T cells proliferate by mRNA of Example 2A in vivo in Test Example 6A.

FIG. 27 illustrates results obtained by evaluating whether an antigen-MHC I complex, CD80, and IL-2 are expressed on cells by mRNA of Example 3A in vivo in Test Example 7A.

FIG. 28 illustrates results obtained by evaluating whether intrinsic Gtf2i-reactive CD8T cells proliferate by mRNA of Example 3A in vivo in Test Example 8A.

FIG. 29 illustrates results obtained by evaluating whether an antigen-MHC II complex, CD80, and IL-12 are expressed on cells by mRNA of Example 4A in vivo in Test Example 9A.

FIG. 30 illustrates results obtained by evaluating whether intrinsic OVA-reactive CD8T cells proliferate by mRNA of Example 4A in vivo in Test Example 10A.

FIG. 31 illustrates results obtained by evaluating whether intrinsic RPL18-reactive CD8T cells proliferate by mRNA of Example 5A in vivo in Test Example 11A.

### DESCRIPTION OF EMBODIMENTS

### Definitions

### Comprising

In the present specification, “comprising” includes “substantially comprising”, “essentially comprising”, “consisting essentially of”, and “consisting of”.

### Extracellular Vesicle

The “extracellular vesicle” used in the present specification is not particularly limited as long as it is a vesicle secreted from cells, and examples thereof include exosomes, microvesicles (MV), and apoptotic bodies.

The “exosome” used in the present specification means a vesicle of about 20 to about 500 nm (preferably about 20 to about 200 nm, more preferably about 25 to about 150 nm, and still more preferably about 30 to about 100 nm), the vesicle being derived from an endocytosis pathway. Examples of constituent components of the exosome include a protein and a nucleic acid (mRNA, miRNA, or non-coated RNA). The exosome has a function of controlling intercellular communication. Examples of a maker molecule of the exosome include Alix, Tsg101, a Tetraspanin, a flotillin, and phosphatidylserine.

The “microvesicle” used in the present specification means a vesicle of about 50 to about 1,000 nm, the vesicle being derived from a cytoplasmic membrane. Examples of constituent components of the microvesicle include a protein and a nucleic acid (mRNA, miRNA, non-coated RNA, or the like). The microvesicle has a function of controlling intercellular communication and the like. Examples of a marker molecule of the microvesicle include integrin, selectin, CD40, and CD154.

The “apoptotic body” used in the present specification means a vesicle of about 500 to about 2,000 nm, the vesicle being derived from a cytoplasmic membrane. Examples of constituent components of the apoptotic body include a fragmented nucleus and a cell organelle. The apoptotic body has a function of inducing phagocytosis and the like. Examples of a maker molecule of the apoptotic body include Annexin V and phosphatidylserine.

The “antigen-presenting extracellular vesicle” used in the present specification means an extracellular vesicle presenting an antigen outside membrane thereof.

### Antigen-Presenting Cell

The “antigen-presenting cell” used in the present specification means a cell presenting one or a plurality of kinds of antigens outside membrane thereof.

In the antigen-presenting cell, it is preferable to present any one or a plurality of kinds of cytokines (such as T-cell stimulatory cytokines as defined below) outside membrane thereof.

In the antigen-presenting cell, the antigen is preferably presented outside the membrane by being immobilized outside the membrane, and more preferably in the form of a fusion molecule fused with a major histocompatibility gene complex molecule as defined below (that is, the antigen is not temporarily attached to the outer membrane).

In the antigen-presenting cell, it is preferable that the antigen peptide and the cytokine are temporarily expressed by introduction of a polynucleotide comprising a sequence encoding one or a plurality of kinds of antigen peptides and a sequence encoding one or a plurality of cytokines, and are simultaneously presented outside the membrane.

Furthermore, it is preferable that any auxiliary signal (for example, a T-cell costimulatory molecule as defined below) is presented outside the membrane in the antigen-presenting cell.

### Major Histocompatibility Gene Complex Molecule

The “major histocompatibility complex (hereinafter, also referred to as “MHC”) molecule” used in the present specification is not particularly limited as long as it has an antigen-binding gap and can bind to an antigen to be presented to a T cell, a T cell precursor, or the like. Examples of the MHC molecule include an MHC class I molecule and an MHC class II molecule. The MHC molecule may be derived from any animal species. Examples thereof include a human leukocyte antigen (HLA) in a human and an H2 system in a mouse.

HLA corresponding to the MHC class I molecule may be classified into subtypes such as HLA-A, HLA-B, HLA-Cw, HLA-F, and HLA-G. Polymorphism (allele) is known for these subtypes. Examples of polymorphism of HLA-A include HLA-A1, HLA-A0201, and HLA-A24, examples of polymorphism of HLA-B include HLA-B7, HLA-B40), and HLA-B4403, and examples of polymorphism of HLA-Cw include HLA-Cw0301, HLA-Cw0401, and HLA-Cw0602.

HLA corresponding to the MHC class II molecule may be classified into subtypes such as HLA-DR, HLA-DQ, and HLA-DP.

The MHC molecule described in the present specification is not limited as long as the function thereof can be exhibited, and an amino acid sequence identity of a wild-type amino acid sequence (for example, in a case of an MHC class I molecule: for example, an MHC class Iα chain of SEQ ID NO: 9 or the like, β₂ microglobulin of SEQ ID NO: 7 or the like, a single chain MHC class I molecule of SEQ ID NO: 65 or the like, and the like; and in a case of an MHC class II molecule: for example, an MHC class IIα chain of SEQ ID NO: 71 or the like, an MHC class IIβ chain of SEQ ID NO: 37 or the like, a single chain MHC class II molecule, and the like) may be 80% or more, preferably 90% or more, more preferably 95% or more, still more preferably 98% or more, and further still more preferably 99% or more. Alternatively, the MHC molecule described in the present specification may be obtained by deletion, insertion, and/or substitution of one or a plurality of amino acids with respect to the wild-type amino acid sequence as long as it can exhibit the function thereof.

The “antigen-presenting MHC molecule” used in the present specification is not particularly limited as long as it is an MHC molecule presenting an antigen, and examples thereof include an antigen-presenting MHC class I molecule and an antigen-presenting MHC class II molecule. Examples of the “antigen-presenting MHC class I molecule” include a complex of an antigen, an MHC class Iα chain or an extracellular domain thereof, and β₂ microglobulin: a complex of an antigen and a single chain MHC class I molecule; a fusion protein in which an antigen and a single chain MHC class I molecule are bound; and a complex an antigen, and a fusion protein of an extracellular domain of an MHC class Iα chain and another protein or a domain thereof or a fragment thereof (for example, a fusion protein of an extracellular domain of an MHC class Iα chain and an Fc portion of an antibody, a fusion protein of an extracellular domain of an MHC class Iα chain and a transmembrane domain of another membrane protein, and the like) Examples of the “antigen-presenting MHC class II molecule” include a complex of an antigen, an MHC class IIα chain or an extracellular domain thereof, and an MHC class IIβ chain or an extracellular domain thereof: a complex of an antigen and a single chain MHC class II molecule: a complex of a fusion protein in which an antigen and an MHC class IIβ chain are bound and an MHC class IIα chain; and a complex of a fusion protein of an antigen, an extracellular domain of an MHC class IIα chain and another protein or a domain thereof or a fragment thereof (for example, a fusion protein of an extracellular domain of an MHC class IIα chain and an Fc portion of an antibody: a fusion protein of an extracellular domain of an MHC class IIα chain and a transmembrane domain of another membrane protein; and the like), and a fusion protein of an extracellular domain of an MHC class IIβ chain and another protein or a domain thereof or a fragment thereof (for example, a fusion protein of an extracellular domain of an MHC class IIβ chain and an Fc portion of an antibody: a fusion protein of an amino acid sequence containing an extracellular domain of an MHC class IIβ chain and a transmembrane domain of another membrane protein; and the like).

The “single chain MHC molecule”, the “single chain MHC class I molecule”, or the “single chain MHC class II molecule” used in the present specification means a fusion protein in which an α chain of an MHC molecule (or an MHC class I molecule or an MHC class II molecule) or an extracellular domain thereof, and a β chain or an extracellular domain thereof or β₂ microglobulin are linked by a spacer sequence, if necessary. Examples of the “single chain MHC class I molecule” include a fusion protein in which an MHC class Iα chain and β₂ microglobulin are linked by a spacer sequence, if necessary. Examples of the “single chain MHC class II molecule” include a fusion protein in which an MHC class IIα chain and an MHC class IIβ chain are linked by a spacer sequence, if necessary.

The “single chain MHC molecule containing a transmembrane domain” used in the present specification means a “single chain MHC molecule” containing a transmembrane domain derived from an MHC molecule (a transmembrane domain of an MHC class Iα chain, an MHC class IIα chain, or an MHC class IIβ chain).

The “protein (or a fusion protein, a protein complex, or the like) which comprises an antigen-presenting MHC molecule and is capable of presenting the antigen (or an antigen peptide) outside membrane” used in the present specification means a protein comprising at least an antigen-presenting MHC molecule and presenting an antigen (or an antigen peptide) outside membrane, in which the protein is capable of presenting an antigen to T cells and the like (a fusion protein, a protein complex, or the like). The “protein (or a fusion protein, a protein complex, or the like) comprising an antigen-presenting MHC molecule and presenting an antigen (or an antigen peptide) outside the membrane” may be expressed in the form of a fusion protein, a protein complex, or the like using a plasmid or the like so that the protein is expressed in membrane of an extracellular vesicle. Alternatively, in a case where a soluble antigen-presenting MHC molecule (although not limited thereto, a fusion protein comprising an MHC class Iα chain and an immunoglobulin heavy chain described in Patent Literature 1: a soluble MHC class I molecule described in JP 2007-161719 A, or the like) is used, the “protein (or a fusion protein, a protein complex, or the like) which comprises an antigen-presenting MHC molecule and is capable of presenting an antigen (or an antigen peptide) outside the membrane” may be a protein in which a soluble antigen-presenting MHC molecule and an extracellular vesicle are bound to membrane of the extracellular vesicle by a lipid linker, a peptide linker, or the like, if necessary (for example, the method described in JP 2018-104341 A or the like may be referred to). Alternatively, the protein may be a protein in which a desired tag (for example, a His tag, a FLAG tag, a PNE tag (SEQ ID NO: 79: NYHLENEVARLKKL), or the like) is added to an N-terminal side or a C-terminal side of a soluble antigen-presenting MHC molecule (for example, the tag may be expressed as a fusion protein together with other components, or may be bound to a separately prepared soluble antigen-presenting MHC molecule by a linker or the like, if necessary), and a protein containing an antibody against the tag or an antigen-binding fragment thereof (for example, scFv, Fab, or a nanobody) (for example, a method using a zPNE tag and an antibody against the tag may be referred to).

### Antigen

The “antigen” used in the present specification is not particularly limited as long as it can have antigenicity, and includes not only peptide antigens but also non-peptide antigens (for example, constituent elements of a bacterial membrane such as mycolic acid and lipoarabinomannan) such as phospholipids and complex carbohydrates.

The “antigen peptide” used in the present specification is not particularly limited as long as it is a peptide that can be an antigen, and may be naturally derived, synthetically derived, or commercially available. Examples of the antigen peptide include, but are not limited to, tumor-associated antigen peptides such as WT-1, an α-fetal protein. MAGE-1, MAGE-3, placental alkaline phosphatase Sialyl-Lewis X, CA-125, CA-19, TAG-72, epithelial glycoprotein 2, 5T4, an α-fetal protein receptor, M2A, tyrosinase. Ras, p53, Her-2/neu, EGF-R, an estrogen receptor, a progesterone receptor, myc, BCR-ABL, HPV-type 16, melanotransferrin, MUC1, CD10, CD19, CD20, CD37, CD45R, an IL-2 receptor a chain, a T cell receptor, prostatic acid phosphatase, GP100, MelanA/Mart-1, gp75/brown, BAGE, S-100, itokeratin, CYFRA21-1, Ep-CAM, Gtf2i and RPL18: self-antigen peptides such as insulin, glutamic acid decarboxylase, ICA512/IA-2 protein tyrosine phosphatase, ICA12, ICA69, preproinsulin, HSP60, carboxypeptidase H. periferin, GM1-2, vitronectin, β-crystallin, carreticulin, serotransferase, keratin, pyruvate carboxylase, C1, billin 2, nucleosome, ribonucleoprotein, myelin oligodendrocyte glycoprotein, myelin-associated glycoprotein, myelin/oligodendrocyte basic protein, oligodendrocyte-specific protein, myelin basic protein, and proteolipid protein: antigen peptides derived from infectious pathogens such as protozoa (for example, plasmodium, leishmania, and trypanosoma), bacteria (for example, gram-positive cocci, gram-positive rods, gram-negative bacteria, and anaerobic bacteria), fungi (for example, Aspergillus, Blastomycosis, Candida, Coccidioidomycosis, Cryptococcus, Histoplasma, Paracoccidioidomycosis, and Sporoslix), viruses (for example, adenovirus, simple herpesvirus, papillomavirus, respiratory synthiavirus, poxvirus, HIV, influenza virus, and coronavirus such as SARS-CoV or SARS-COV2), intracellular parasites (for example, Chlamydiaceae, Mycoplasmataceae, Acholeplasma, and Rickettsiaceae), and helminths (for example, nematodes, trematodes, and tapeworms); and other antigen peptides such as prion.

The antigen peptide may comprise an allergen that causes allergic symptoms. Examples of the allergen include exogenous peptides such as peptides derived from house dust, mites, animals (for example, companion animals such as cats and dogs), and pollens (for example, Japanese cedar or Japanese cypress), in addition to the peptides derived from protozoa, bacteria, fungi, intracellular parasites, and helminths. More specifically, proteins contained in Japanese cedar such as Cryj1 are exemplified. Alternatively, the allergen that causes allergic symptoms may be derived from food. Examples of the allergen that causes allergic symptoms for food include peptides derived from chicken egg, cow milk, wheat, buckwheat, crab, shrimp, and peanut.

The “MHC molecule-restricted antigen peptide” used in the present specification means an antigen peptide capable of binding to an MHC molecule in vitro, in vivo, and/or ex vivo. The number of amino acid residues of the “MHC molecule-restricted antigen peptide” is usually about 7 to about 30. Examples of the “MHC molecule-restricted antigen peptide” include an MHC class I molecule-restricted antigen peptide and an MHC class II molecule-restricted antigen peptide.

The “MHC class I molecule-restricted antigen peptide” used in the present specification means an antigen peptide capable of binding to an MHC class I molecule in vitro, in vivo, and/or ex vivo. When the MHC class I molecule-restricted antigen peptide is presented outside membrane of the extracellular vesicle, for example, the antigen peptide is recognized by precursor T cells or the like, and cytotoxic T cells or the like can be induced. The number of amino acid residues of the “MHC class I molecule-restricted antigen peptide” is usually about 7 to about 30, preferably about 7 to about 25, more preferably about 7 to about 20, still more preferably about 7 to about 15, and further still more about 7 to about 12.

The “MHC class II molecule-restricted antigen peptide” used in the present specification means an antigen peptide capable of binding to an MHC class II molecule in vitro, in vivo, and/or ex vivo. When the MHC class II molecule-restricted antigen peptide is presented outside membrane of the extracellular vesicle, for example, the antigen peptide is recognized by precursor T cells or the like, and α-T cells or the like can be induced. The number of amino acid residues of the “MHC class II molecule-restricted antigen peptide” is usually about 7 to about 30, preferably about 10 to about 25, and more preferably about 12 to about 24.

The “MHC molecule-restricted antigen peptide”, the “MHC class I molecule-restricted antigen peptide”, or the “MHC class II molecule-restricted antigen peptide” is not particularly limited as long as it is an antigen peptide capable of binding to an MHC molecule, an MHC class I molecule, or an MHC class II molecule.

### T-Cell Stimulatory Cytokine

The “T-cell stimulatory cytokine” used in the present specification is not particularly limited as long as it is a cytokine capable of stimulating (for example, activating, suppressing, or the like) T cells via a receptor or the like expressed on the membrane of the T cell. Examples of the T-cell stimulatory cytokine include, are not limited to, IL-2, IL-4, IL-6, IL-12, IL-15, TGF-β, IFN-α, and IFN-γ. Among them, a T-cell stimulatory cytokine capable of forming a multimer of homo or hetero subunits (for example, IL-12, TGF-β, or the like) may be a T-cell stimulatory cytokine comprising a continuous amino acid sequence linked by a peptide linker or the like, if necessary, as long as it is functional (that is, as long as it can have a desired pharmacological activity). The T-cell stimulatory cytokine may be bound to or fused with other full-length proteins or partial sequence peptides thereof (for example, a Sushi domain of an IL-15 receptor) as long as it maintains the ability to stimulate T cells.

The T-cell stimulatory cytokines described in the present specification may be derived from any animal species. Examples of the T-cell stimulatory cytokine include T-cell stimulatory cytokines derived from animals such as mammals, for example, rodents such as a mouse and a rat: lagomorph such as a rabbit, ungulates such as a pig, a cow, a goat, a horse, and a sheep: carnivora such as a dog and a cat; and primates such as a human, a monkey, a rhesus monkey, a crab-eating macaque, a marmoset, an orangutan, and a chimpanzee. The T-cell stimulatory cytokine described in the present specification is preferably derived from rodents or primates, and more preferably derived from a mouse or a human.

The T-cell stimulatory cytokine described in the present specification may have an amino acid sequence identity of 80% or more, preferably 90% or more, more preferably 95% or more, still more preferably 98% or more, and further still more preferably 99% or more with respect to a wild-type amino acid sequence thereof (for example, in the case of IL-2, for example, SEQ ID NO: 25 or the like; and in the case of IL-4, for example, SEQ ID NO: 53 or the like), as long as it can exhibit the function thereof. Alternatively, the T-cell stimulatory cytokine described in the present specification may be obtained by deletion, insertion, and/or substitution of one or a plurality of amino acids with respect to the wild-type amino acid sequence as long as it can exhibit the function thereof.

The “protein which comprises a (first or second) T-cell stimulatory cytokine and is capable of presenting the (first or second) T-cell stimulatory cytokine outside membrane” used in the present specification means a protein which comprises at least a T-cell stimulatory cytokine and is capable of presenting the T-cell stimulatory cytokine outside membrane of a cell or an extracellular vesicle. The “protein which comprises a (first or second) T-cell stimulatory cytokine and is capable of presenting the (first or second) T-cell stimulatory cytokine outside membrane” may be expressed by using a plasmid or the like as a fusion protein having a fragment comprising a T-cell stimulatory cytokine and a membrane protein or a transmembrane domain thereof so that the protein is expressed in the membrane of the cell or the extracellular vesicle.

Alternatively, in a case where a soluble T-cell stimulatory cytokine (examples thereof include, but are not limited to, a T-cell stimulatory cytokine itself: a fusion protein of a T-cell stimulatory cytokine and an Fc portion of an antibody; and a complex of a T-cell stimulatory cytokine and an antibody that recognizes the T-cell stimulatory cytokine or an antigen-binding fragment thereof (for example, scFv, Fab, or a nanobody)) is used, the “protein which comprises a (first or second) T-cell stimulatory cytokine and is capable of presenting the (first or second) T-cell stimulatory cytokine outside membrane” may be a protein in which a soluble T-cell stimulatory cytokine and a cell or an extracellular vesicle are bound to membrane of an extracellular vesicle by a lipid linker, a peptide linker, or the like, if necessary (for example, the method described in JP 2018-104341 A or the like may be referred to). Alternatively, the protein may be a mixture of a protein in which a desired tag (for example, a His tag, a FLAG tag, or a PNE tag) is added to the N-terminus or C-terminus of a soluble T-cell stimulatory cytokine (the tag may be expressed as a fusion protein together with other constituent elements, for example, may be bound to an additionally prepared soluble T-cell stimulatory cytokine by a linker or the like, if necessary), and a cell or an extracellular vesicle containing a protein comprising an antibody against the tag or an antigen-binding fragment thereof (for example, scFv, Fab, or a nanobody) (for example, an antibody itself against the tag or an antigen-binding fragment thereof (for example, scFv, Fab, or a nanobody) bound to the membrane of the cell or the extracellular vesicle by a linker or the like, if necessary: a fusion protein in which a nanobody for the tag is bound to the N-terminus or C-terminus of a membrane protein capable of being expressed in membrane of a cell or an extracellular vesicle or a transmembrane domain thereof) in membrane under desired conditions (for example, the method using a PNE tag and an antibody against the tag described in Raj D. et al., Gut., 2019 June: 68 (6): 1052-1064, and the like, may be referred to). Note that in a case of a T-cell 30) stimulatory cytokine formed by multimers of subunits, when one of the subunits is a protein that can be presented outside membrane of a cell or an extracellular vesicle, the remaining subunits do not need to be in a form that can be presented outside the membrane. When one of the subunits is a protein capable of being presented outside membrane of an extracellular vesicle, a functional T-cell stimulatory cytokine can be constructed outside the membrane of the extracellular vesicle by adding or co-expressing other subunits.

### T-Cell Costimulatory Molecule

The “T-cell costimulatory molecule” used in the present specification means a molecule that can contribute to activation of T cells by interacting with a molecule present on membrane of a T cell such as CD28 or CD134. Examples of the T-cell costimulatory molecule include, but are not limited to, molecules such as CD80 and CD86, or extracellular domains thereof or functional fragments thereof: antibodies such as an anti-CD28 antibody and an anti-CD134 antibody or antigen-binding fragments thereof (for example, scFv, Fab, or a nanobody); and a fusion protein (or a complex or an aggregate) of them with a transmembrane domain of another protein or an Fc portion of an antibody.

The “T-cell costimulatory molecule containing a transmembrane domain” used in the present specification means a “T-cell costimulatory molecule” that further contains a transmembrane domain derived from a T-cell costimulatory molecule.

The T-cell costimulatory molecule described in the present specification may be derived from any animal species. Examples of the T-cell costimulatory molecule include T-cell costimulatory molecules derived from animals such as mammals, for example, rodents such as a mouse, a rat, a hamster, and a guinea pig: lagomorph such as a rabbit: ungulates such as a pig, a cow, a goat, a horse, and a sheep: carnivora such as a dog and a cat; and primates such as a human, a monkey, a rhesus monkey, a crab-eating macaque, a marmoset, an orangutan, and a chimpanzee. The T-cell costimulatory molecule described in the present specification is preferably derived from rodents or primates, and more preferably derived from a mouse or a human.

The T-cell costimulatory molecule described in the present specification may have an amino acid sequence identity of 80% or more, preferably 90% or more, more preferably 95% or more, still more preferably 98% or more, and further still more preferably 99% or more with respect to a wild-type amino acid sequence thereof (for example, in the case of CD80, for example, SEQ ID NO: 67 or the like), as long as it can exhibit the function described above. Alternatively, the T-cell costimulatory molecule described in the present specification may be obtained by deletion, insertion, and/or substitution of one or a plurality of amino acids with respect to the wild-type amino acid sequence as long as it can exhibit the function thereof.

The “protein which comprises a T-cell costimulatory molecule and is capable of allowing the T-cell costimulatory molecule to interact with T cells” used in the present specification means a protein which comprises at least a T-cell costimulatory molecule and is capable of interacting with a molecule present in membrane of the T cell. That is, it means that the at least a portion capable of interacting with T cells present in the T-cell costimulatory molecule is located outside the membrane of the cell or the extracellular vesicle. The “protein which comprises a T-cell costimulatory molecule and is capable of allowing the T-cell costimulatory molecule to interact with T cells” may be expressed by using a plasmid or the like so that it is expressed in the membrane of the cell or the extracellular vesicle. Alternatively, in a case where a soluble T-cell costimulatory molecule (examples thereof include, but are not limited to, a fusion protein of an extracellular domain of CD80 and an Fc portion of an antibody; and an anti-CD28 antibody or an antigen-binding fragment thereof (for example, scFv. Fab, or a nanobody)) is used, the “protein which comprises a T-cell costimulatory molecule and is capable of allowing the T-cell costimulatory molecule to interact with T cells” may be a protein in which a soluble T-cell costimulatory molecule and a cell or an extracellular vesicle are bound to membrane of the cell or the extracellular vesicle by a lipid linker, a spacer sequence, or the like, if necessary (for example, the method described in JP 2018-104341 A or the like may be referred to). Alternatively, the protein may be a mixture of a protein in which a desired tag (for example, a His tag, a FLAG tag, or a PNE tag) is added to the N-terminus or C-terminus of a soluble T-cell costimulatory molecule (the tag may be expressed as a fusion protein together with other constituent elements, for example, may be bound to an additionally prepared soluble T-cell costimulatory molecule by a linker or the like, if necessary), and a cell or an extracellular vesicle containing a protein comprising an antibody against the tag or an antigen-binding fragment thereof (for example, scFv, Fab, or a nanobody) (for example, an antibody itself against the tag or an antigen-binding fragment thereof (for example, scFv, Fab, or a nanobody) bound to the membrane of the cell or the extracellular vesicle by a linker or the like, if necessary: a fusion protein in which a nanobody for the tag is bound to the N-terminus or C-terminus of a membrane protein capable of being expressed in membrane of a cell or an extracellular vesicle or a transmembrane domain thereof) in membrane under desired conditions (for example, the method using a PNE tag and an antibody against the tag described in Raj D. et al., Gut., 2019 June: 68 (6): 1052-1064, and the like, may be referred to).

As the “membrane protein capable of being expressed in membrane of a cell or the transmembrane domain thereof” used in the present specification, any membrane protein or a transmembrane domain thereof can be selected as long as it can be expressed in the membrane of the cell.

Although not limited thereto, the membrane protein or the transmembrane domain thereof preferably includes a part or all of CD8, CD4, CD28, a transferrin receptor, and the like, and a part or all of an FC region of a membrane-bound immunoglobulin molecule such as IgG1, IgG2, or IgG4.

As the “protein capable of” binding to membrane of a cell or the domain thereof used in the present specification, any protein or a domain thereof can be selected as long as it can be bound to the membrane of the cell.

As the “membrane protein capable of being expressed in membrane of an extracellular vesicle or the transmembrane domain thereof” used in the present specification, any membrane protein or a transmembrane domain thereof can be selected as long as it can be expressed in the membrane of the extracellular vesicle.

The “membrane protein capable of being expressed in membrane of an extracellular vesicle or a transmembrane domain thereof” is preferably a membrane protein known to be capable of being expressed in an extracellular vesicle (for example, exosome or the like) (for example, a Tetraspanin, CD58, ICAM-1, PTGFRN (for example, see Non Patent Literature 1, WO 2019/183578 A, and the like), and the like), or a transmembrane domain thereof.

As the “protein capable of binding to membrane of an extracellular vesicle or the domain thereof” used in the present specification, any protein or a domain thereof can be selected as long as it can be bound in the membrane of the extracellular vesicle. The “protein capable of” binding to membrane of an extracellular vesicle or the domain thereof is preferably a protein known to be capable of binding to membrane of an extracellular vesicle (for example, exosome or the like) (for example, MFG-E8 or a domain thereof (for example, a CI or C2 domain of MFG-E8 described in Alain Delcayre, et al., Blood Cells, Molecules, and Diseases 35 (2005) 158-168)).

The “membrane protein capable of being expressed in membrane of a cell or an extracellular vesicle or the transmembrane domain thereof” or the “protein capable of binding to membrane of a cell or an extracellular vesicle or the domain thereof” described in the present specification may be derived from any animal species. Examples of the T-cell stimulatory cytokine include T-cell stimulatory cytokines derived from animals such as mammals, for example, rodents such as a mouse and a rat; lagomorph such as a rabbit, ungulates such as a pig, a cow, a goat, a horse, and a sheep; carnivora such as a dog and a cat; and primates such as a human, a monkey, a rhesus monkey, a crab-eating macaque, a marmoset, an orangutan, and a chimpanzee. The “membrane protein capable of being expressed in membrane of a cell or an extracellular vesicle or the transmembrane domain thereof” or the “protein capable of binding to membrane of a cell or an extracellular vesicle or the domain thereof” described in the present specification is preferably derived from rodents or primates, and is more preferably derived from a mouse or a human.

According to Non Patent Literature 2, markers of mammalian extracellular vesicles are classified as follows.

Examples of a membrane protein or a GPI anchor protein that can be used as a marker protein of an extracellular vesicle include:

### 1) Tissue Non-Specific

Tetraspanins (CD63, CD9, CD81, and CD82), other multiple transmembrane type membrane proteins (CD47 and hetero trimer G proteins (guanine nucleotide-binding proteins (GNA)),
MHC class I (HLA-A/B/C,H2-K/D/Q),
integrin (ITGA/ITGB), a transferrin receptor (TFR2);
LAMP1/2;
heparan sulfate proteoglycans ((including syndecan (SDC));
extracellular matrix metalloprotease inducer (EMMPRIN) (also referred to as BSG or CD147);
ADAM10;
CD73 that is a GPI anchored 5′ nucleotidase (NT5E),
CD55 and CD59 that are GPI anchored complement binding proteins; and sonic hedgehog protein (SHH); and

### 2) Cell/Tissue Specific

several Tetraspanins: TSPAN8 (epithelial specific), CD37, and CD53 (leukocyte-specific);
PECAM1 (endothelial specific);
ERBB2 (breast cancer specific);
EPCAM (epithelial specific);
CD90 (THY1) (mesenchymal stem cell-specific):
CD45 (PTPRC) (immune cell-specific), CD41 (ITGA2B), or CD42a (GP9) (platelet-specific);
glycophorin A (GYPA) (erythroid specific):
CD14 (monocyte specific), MHC class II (HLA-DR/DP/DQ,H2-A); CD3 (T cell specific);
acetylcholinesterase/AChE-S(neuronal cell-specific), AChE-E (erythroid specific); and
amyloid βA4/APP (neuronal cell-specific).

Therefore, although not limited thereto, a protein that is a marker of an extracellular vesicle may be used as the “membrane protein capable of being expressing in membrane of an extracellular vesicle” or the “the protein capable of binding to membrane of an extracellular vesicle” in the present invention.

The “Tetraspanin” used in the present specification means a protein belonging to a Tetraspanin family (for example, but are not limited to, CD9, CD53, CD63, CD81, CD82, CD151, and the like). The Tetraspanin usually contains, from an N-terminal side thereof, a transmembrane domain 1 (hereinafter, referred to as “TM1”), a small extracellular loop (hereinafter, referred to as “SEL”), a transmembrane domain 2 (hereinafter, referred to as “TM2”), a small intracellular loop (hereinafter, referred to as “SIL”), a transmembrane domain 3 (hereinafter, referred to as “TM3”), a large extracellular loop (hereinafter, referred to as “LEL”), and a transmembrane domain 4 (hereinafter, referred to as “TM4”), and thus is a quadruple transmembrane type, and both the N-terminus and the C-terminus are present on the cytoplasmic side. For example, in a case where the Tetraspanin is mouse CD63 (amino acid sequences: 1 to 238, SEQ ID NO: 27), the Tetraspanin may typically contain TM1, SEL, TM2, SIL, and TM3 in the amino acid sequence from about 1 to about 110, LEL in the amino acid sequence from about 111 to about 200, and TM4 in the amino acid sequence from about 201 to about 238.

Each domain (for example, TM1, SEL, SIL, LTL, or the like) in the “Tetraspanin” described in the present specification may be derived from the same Tetraspanin, or may be derived from different Tetraspanins in whole or in part. The Tetraspanin described in the present specification may have an amino acid sequence identity of 80% or more, preferably 90% or more, more preferably 95% or more, still more preferably 98% or more, and further still more preferably 99% or more with respect to a wild-type amino acid sequence thereof (for example, in the case of CD9 with a full length, for example, SEQ ID NO: 21 or the like; in the case of CD63 with a full length, for example, SEQ ID NO: 27 or the like; and in the case of CD81 with a full length, for example, SEQ ID NO: 15 or the like), as long as it can be expressed in the membrane of the extracellular vesicle. Alternatively, the Tetraspanin described in the present specification may be obtained by deletion, insertion, and/or substitution of one or a plurality of amino acids with respect to the wild-type amino acid sequence as long as it can be expressed in the membrane of the extracellular vesicle.

A partial sequence of the Tetraspanin (for example, each domain: a partial sequence containing TM1, SEL, TM2, SIL, and TM3 (for example, in the case of CD63, SEQ ID NO: 57 or the like; and in the case of CD81, SEQ ID NO: 61 or the like): a partial sequence containing TM4 (for example, in the case of CD63, SEQ ID NO: 59 or the like; and in the case of CD81, SEQ ID NO: 63 or the like)) described in the present specification may have an amino acid sequence identity of 80% or more, preferably 90% or more, more preferably 95% or more, still more preferably 98% or more, and further still more preferably 99% or more with respect to a wild-type amino acid sequence thereof. Alternatively, the partial sequence of the Tetraspanin described in the present specification may be obtained by deletion, insertion, and/or substitution of one or a plurality of amino acids with respect to the wild-type amino acid sequence.

MFG-E8 described in the present specification may have an amino acid sequence identity of 80% or more, preferably 90% or more, more preferably 95% or more, still more preferably 98% or more, and further still more preferably 99% or more with respect to a wild-type amino acid sequence thereof (for example, SEQ ID NO: 49 or the like), as long as it can bind to the membrane of the extracellular vesicle. Alternatively, MFG-E8 described in the present specification may be obtained by deletion, insertion, and/or substitution of one or a plurality of amino acids with respect to the wild-type amino acid sequence as long as it can bind to the membrane of the extracellular vesicle.

CD58, PTGFRN, or the like described in the present specification may have an amino acid sequence identity of 80% or more, preferably 90% or more, more preferably 95% or more, still more preferably 98% or more, and further still more preferably 99% or more with respect to a wild-type amino acid sequence thereof, as long as it can be expressed in the membrane of the extracellular vesicle or can bind to the membrane of the extracellular vesicle. Alternatively, CD58, PTGFRN, or the like described in the present specification may be obtained by deletion, insertion, and/or substitution of one or a plurality of amino acids with respect to the wild-type amino acid sequence as long as it can be expressed in the membrane of the extracellular vesicle or can bind to the membrane of the extracellular vesicle.

### Spacer Sequence

The “spacer sequence” used in the present specification means any sequence having at least one amino acid residue that is present between two or more proteins or partial sequences or domains thereof. The spacer sequence can be used, for example, when two or more proteins or partial sequences or domains thereof are linked. The spacer sequence contains a peptide linker. A length of the amino acid residue of the spacer sequence is usually 1 to about 50, preferably about 2 to about 28, and more preferably about 4 to about 25. Examples of the spacer sequence include, but are not limited to, (GGGXS)ₙGₘ (wherein, X is independently A or G each time it appears, n is 1 to 8, and n, and m is 0 to 3) (for example, SEQ ID NO: 5, 11, 29, 39, or the like); (GGGS)ₙGₘ (wherein, n is 1 to 10, and m is 0 to 3); and TₐS_{b}(GGX)ₙGₘ (wherein, X is independently S or T each time it appears, n is 1 to 8, m is 0 to 3, a is 0 or 1, and b is 0 or 1) (for example, SEQ ID NO: 77 or the like).

### Antigen-Presenting Extracellular Vesicles Described in Present Specification

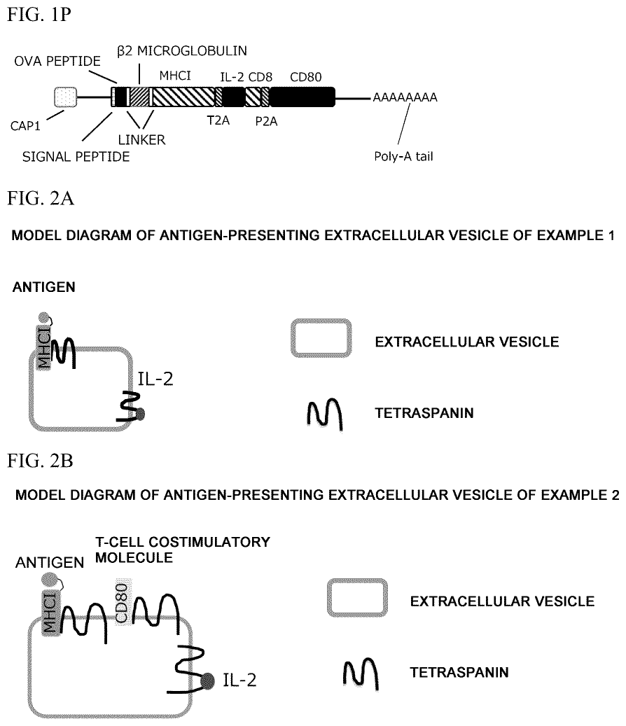

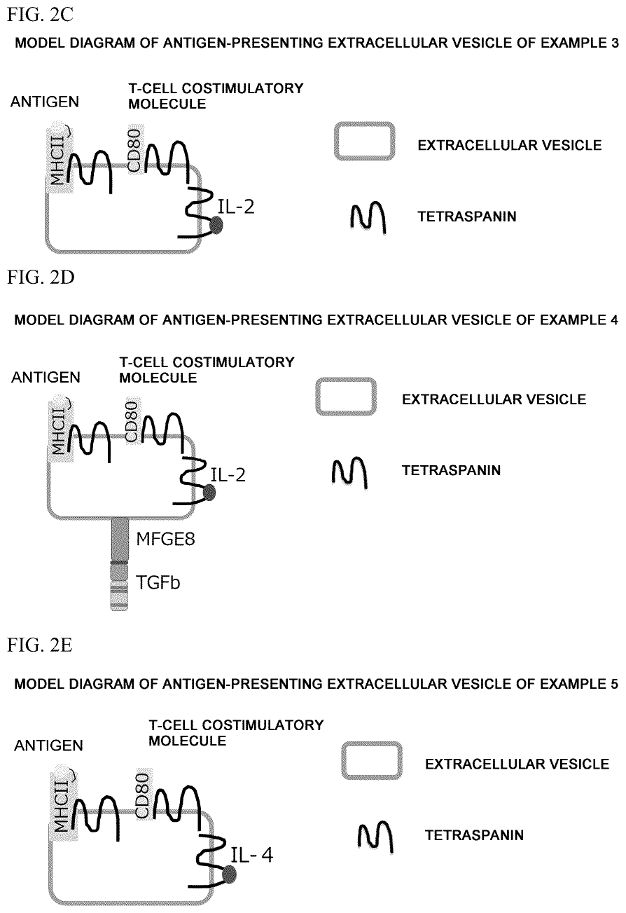

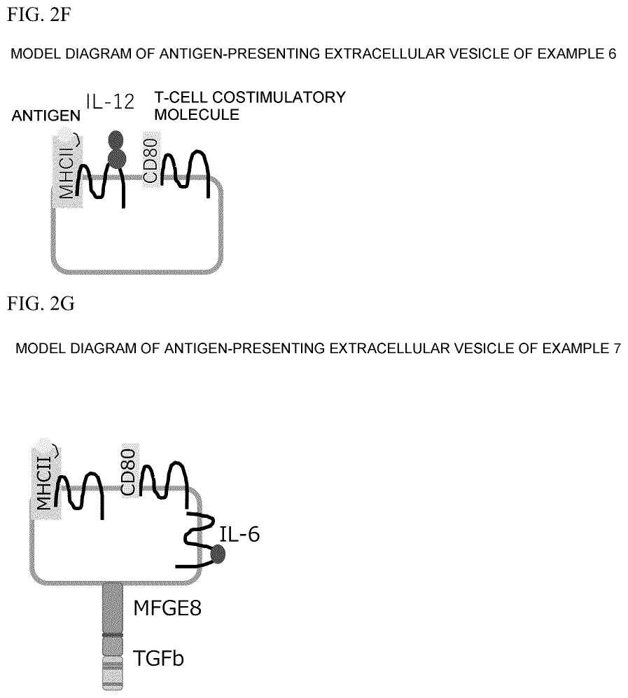

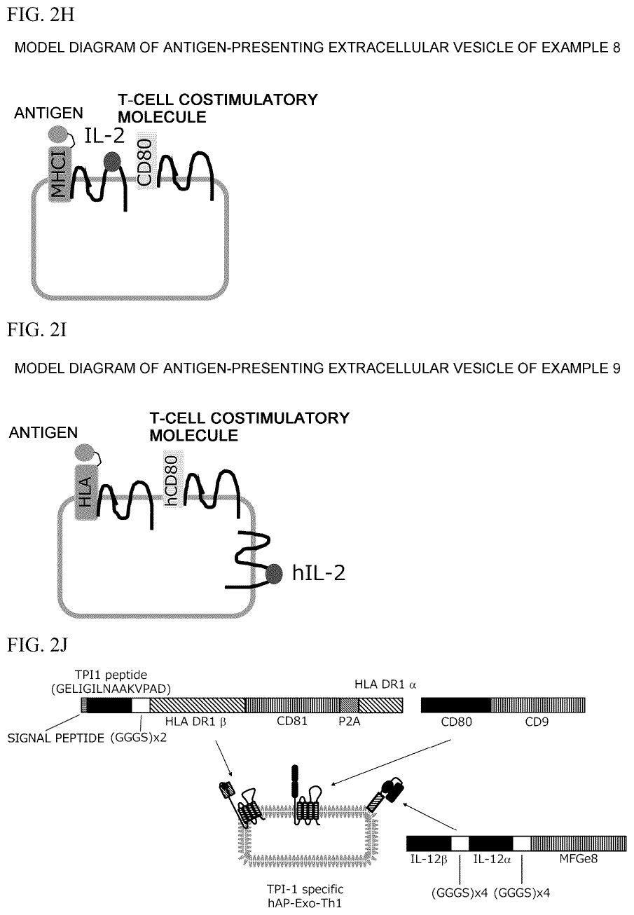

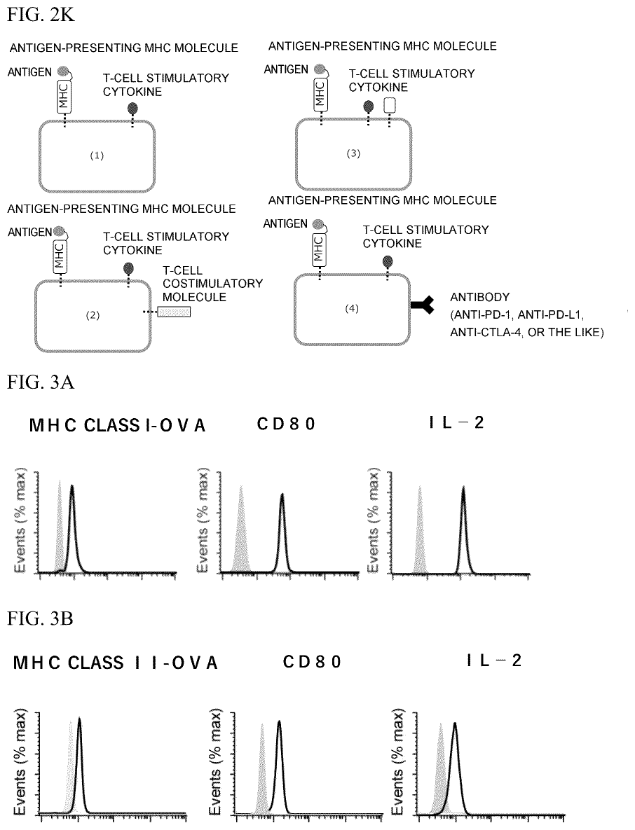

In an embodiment of the present invention, there is provided an extracellular vesicle presenting an antigen-presenting MHC molecule and a T-cell stimulatory cytokine outside membrane (the model is illustrated in (1) of FIG. 2K).

Such an extracellular vesicle may present an antigen-presenting MHC molecule and a T-cell stimulatory cytokine outside membrane thereof by containing proteins specified in the following (A) and (B), or may present an antigen-presenting MHC molecule and a T-cell stimulatory cytokine outside membrane thereof by containing a protein specified in (D).

Alternatively, an antigen-presenting MHC molecule and a T-cell stimulatory cytokine may be attached to membrane surface of an isolated extracellular vesicle later. An attachment method is not particularly limited, an antigen-presenting MHC molecule and a T-cell stimulatory cytokine may be attached to membrane surface by binding each phospholipid to an antigen-presenting MHC molecule and a T-cell stimulatory cytokine and incorporating a phospholipid moiety into membrane of an extracellular vesicle. Phosphatidylserine is present on the surface of the extracellular vesicle. Therefore, each protein obtained by fusing an antigen-presenting MHC molecule or a T-cell stimulatory cytokine desired to be presented to MFG-E8 binding to phosphatidylserine is synthesized and purified, and the fusion protein and an extracellular vesicle are mixed, such that an extracellular vesicle presenting an antigen-presenting MHC molecule and a T-cell stimulatory cytokine on membrane surface can be prepared. In addition, an antigen-presenting MHC molecule to which a PNEtag is attached and a T-cell stimulatory cytokine may be added later to an extracellular vesicle pre-expressing a peptide neoepitope (PNE) nanobody to be presented on membrane surface of the extracellular vesicle. A biotinylated antigen-presenting MHC molecule and a T-cell stimulatory cytokine may be added to the extracellular vesicle expressing streptavidin to be presented on the membrane surface of the extracellular vesicle.

In an embodiment of the present invention, the extracellular vesicle may present a plurality of kinds (2, 3, 4, and 5 kinds) of antigen-presenting MHC molecules and a plurality of kinds (2, 3, 4, and 5 kinds) of T-cell stimulatory cytokines (in order to identify each T-cell stimulatory cytokine, hereinafter, it may be referred to as a first T-cell stimulatory cytokine, a second T-cell stimulatory cytokine, third or higher T-cell stimulatory cytokines, and the like) outside the membrane. Alternatively, the extracellular vesicle may be an extracellular vesicle presenting one kind of an antigen-presenting MHC molecule and a plurality of kinds of cell stimulatory cytokines outside membrane (a model of an extracellular vesicle presenting one kind of an antigen-presenting MHC molecule and two kinds of T-cell stimulatory cytokines outside membrane is illustrated in (3) of FIG. 2K).

In an embodiment of the present invention, there is provided a cell presenting an antigen-presenting MHC molecule and a T-cell stimulatory cytokine outside membrane (corresponding to the model of the extracellular vesicle illustrated in (1) of FIG. 2K).

Such an antigen-presenting cell may present an antigen-presenting MHC molecule and a T-cell stimulatory cytokine outside membrane thereof by containing proteins defined as the following (A) and (B), or may present an antigen-presenting MHC molecule and a T-cell stimulatory cytokine outside membrane thereof by containing a protein defined as (D).

In an embodiment of the present invention, the cell may present a plurality of kinds (2, 3, 4, and 5 kinds) of antigen-presenting MHC molecules and a plurality of kinds (2, 3, 4, and 5 kinds) of T-cell stimulatory cytokines (in order to identify each T-cell stimulatory cytokine, hereinafter, it may be referred to as a first T-cell stimulatory cytokine, a second T-cell stimulatory cytokine, third or higher T-cell stimulatory cytokines, and the like) outside the membrane. Alternatively, the cell may be a cell that presents one kind of antigen-presenting MHC molecule and a plurality of kinds of cell stimulatory cytokines outside membrane (corresponding to the model of the extracellular vesicle illustrated in (3) of FIG. 2K).

In an embodiment of the present invention, there is provided an antigen-presenting cell or an antigen-presenting extracellular vesicle, the membrane of which contains:
(A) a protein which comprises an antigen-presenting MHC molecule and is capable of presenting the antigen outside membrane; and
(B) a protein which comprises a first T-cell stimulatory cytokine and is capable of presenting the first T-cell stimulatory cytokine outside membrane.

### Constitutional Requirement (A)

The “protein which comprises an antigen-presenting MHC molecule and is capable of presenting the antigen outside membrane” of the (A) above may contain another protein or a domain thereof, or the like in addition to the antigen-presenting MHC molecule as long as it is a protein capable of presenting an antigen outside membrane of a cell or an extracellular vesicle.

In an embodiment of the present invention, the (A) above is a fusion protein or a protein complex which comprises an antigen-presenting MHC molecule, and a membrane protein capable of being expressed in membrane of a cell or an extracellular vesicle or a transmembrane domain thereof or a protein capable of binding to membrane of a cell or an extracellular vesicle or a domain thereof, and is capable of presenting the antigen outside membrane.

In an embodiment of the present invention, the (A) above is
(A) a fusion protein capable of presenting an antigen peptide outside membrane, in which the fusion protein comprises an amino acid sequence containing, from an N-terminal side thereof,
(A-1) an amino acid sequence of an MHC molecule-restricted antigen peptide,
(A-2) a spacer sequence which may be optionally present, and
(A-3) an amino acid sequence of a single chain MHC molecule containing a transmembrane domain, in this order, or
(A) a protein complex capable of presenting an antigen peptide outside membrane, in which the protein complex contains, a fusion protein which comprises an amino acid sequence containing, from an N-terminal side thereof,
(A-1) an amino acid sequence of an MHC molecule-restricted antigen peptide,
(A-2) a spacer sequence which may be optionally present, and
(A-3) an amino acid sequence consisting of an MHC class Iα chain, a fusion protein of β₂ microglobulin and an MHC class Iα chain, an MHC class IIα chain, or an MHC class IIβ chain, and
a protein which comprises
(A-6) an amino acid sequence of β₂ microglobulin, an MHC class Iα chain, an MHC class IIβ chain, or an MHC class IIα chain,
wherein an MHC class I molecule or an MHC class II molecule are constituted by a combination of (A-3) and (A-6).

In an embodiment of the present invention, the (A) above is
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of a single chain MHC class I molecule containing a transmembrane domain, in this order.

In addition, in an embodiment of the present invention, the (A) above is
(A) a fusion protein capable of presenting an antigen peptide outside membrane, in which the fusion protein comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of a single chain MHC class II molecule containing a transmembrane domain, in this order.

In an embodiment of the present invention, the (A) above is
(A) a protein complex capable of presenting an antigen peptide outside membrane, in which the protein complex comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of a fusion protein of β₂ microglobulin and an MHC class Iα chain, in this order.

In addition, in an embodiment of the present invention, the (A) above is
(A) a protein complex capable of presenting an antigen peptide outside membrane, in which the protein complex contains a fusion protein which comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of an MHC class Iα chain, in this order, and a protein which comprises
(A-6) an amino acid sequence of β₂ microglobulin.

In addition, in an embodiment of the present invention, the (A) above is
(A) a protein complex capable of presenting an antigen peptide outside membrane, in which the protein complex contains a fusion protein which comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of an MHC class IIβ chain, in this order, and a protein which comprises
(A-6) an amino acid sequence of an MHC class IIα chain.

In addition, in an embodiment of the present invention, the (A) above is
(A) a protein complex capable of presenting an antigen peptide outside membrane, in which the protein complex contains a fusion protein which comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of an MHC class IIα chain, in this order, and
a protein which comprises
(A-6) an amino acid sequence of an MHC class IIβ chain.

In an embodiment of the present invention, the (A) above is a fusion protein or a protein complex which comprises an antigen-presenting MHC molecule, and a Tetraspanin or a transmembrane domain thereof or MFG-E8 or a domain thereof, and is capable of presenting the antigen outside membrane.

In an embodiment of the present invention, the (A) above is
a protein complex containing:
a fusion protein which comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of a single chain MHC molecule,
(A-4) a spacer sequence which may be optionally present, and
(A-5) an amino acid sequence of a Tetraspanin or a membrane-binding domain thereof, in this order, or
a fusion protein which comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of a domain outside membrane of an MHC class Iα chain, β₂ microglobulin, a domain membrane of an MHC class IIα chain, or a domain outside membrane of an MHC class IIβ chain,
(A-4) a spacer sequence which may be optionally present, and
(A-5) an amino acid sequence of a Tetraspanin or a transmembrane domain thereof, in this order; and a protein which comprises
(A-6) an amino acid sequence of β₂ microglobulin, an MHC class Iα chain, an MHC class IIβ chain, or an MHC class IIα chain.

In an embodiment of the present invention, the (A) above is
a fusion protein capable of presenting the antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of a single chain MHC class I molecule,
(A-4) a spacer sequence which may be optionally present, and
(A-5) an amino acid sequence of a Tetraspanin or a transmembrane domain thereof, in this order.

In addition, an embodiment of the present invention, the (A) above is a fusion protein capable of presenting the antigen peptide outside membrane, in which the fusion protein comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of a single chain MHC class II molecule,
(A-4) a spacer sequence which may be optionally present, and
(A-5) an amino acid sequence of a Tetraspanin or a transmembrane domain thereof, in this order.

In an embodiment of the present invention, the (A) above is
a protein complex containing
a fusion protein which comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of β₂ microglobulin,
(A-4) a spacer sequence which may be optionally present, and
(A-5) an amino acid sequence of a Tetraspanin or a transmembrane domain thereof, in this order, and
a protein which comprises
(A-6) an amino acid sequence of an MHC class Iα chain.

In addition, in an embodiment of the present invention, the (A) above is
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of a domain outside membrane of an MHC class Iα chain,
(A-4) a spacer sequence which may be optionally present, and
(A-5) an amino acid sequence of a Tetraspanin or a transmembrane domain thereof, in this order, and
a protein which comprises
(A-6) an amino acid sequence of β₂ microglobulin.

In addition, in an embodiment of the present invention, the (A) above is
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of a domain outside membrane of an MHC class IIβ chain,
(A-4) a spacer sequence which may be optionally present, and
(A-5) an amino acid sequence of a Tetraspanin or a transmembrane domain thereof, in this order, and
a protein which comprises
(A-6) an amino acid sequence of an MHC class IIα chain.

In addition, in an embodiment of the present invention, the (A) above is
(A) a protein complex capable of presenting an antigen peptide outside membrane, in which the protein complex contains
a fusion protein which comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of a domain outside membrane of an MHC class IIα chain,
(A-4) a spacer sequence which may be optionally present, and
(A-5) a Tetraspanin, in this order, and
a protein which comprises
(A-6) an amino acid sequence of an MHC class IIβ chain.

In an embodiment of the present invention, in a case where the “single chain MHC molecule” is a “single chain MHC class I molecule”, the “single chain MHC class I molecule” consists of, from an N-terminal side thereof, β₂ microglobulin (for example, SEQ ID NO: 7 or the like, or a sequence having an amino acid sequence identity thereto of 80% or more, preferably 90% or more, more preferably 95% or more, still more preferably 98% or more, and further still more preferably 99% or more), a spacer sequence which may be optionally present (when present, for example, SEQ ID NOS: 5, 11, 29, 39, 77, and the like), and an MHC class Iα chain (for example, SEQ ID NO: 9 or the like, or a sequence having an amino acid sequence identity thereto of 80% or more, preferably 90% or more, more preferably 95% or more, still more preferably 98% or more, and further still more preferably 99% or more). In an embodiment of the present invention, in a case where the (A-3) above is a “single chain MHC class I molecule”, the “single chain MHC class I molecule” contains SEQ ID NO: 65 or a sequence having an amino acid sequence identity thereto of 80% or more, preferably 90% or more, more preferably 95% or more, still more preferably 98% or more, and further still more preferably 99% or more.

In an embodiment of the present invention, in a case where the “single chain MHC molecule” is a “single chain MHC class II molecule”, the “single chain MHC class II molecule” consists of, from an N-terminal side thereof, an MHC class IIβ chain, a spacer sequence which may be optionally present, and an MHC class IIα chain.

In an embodiment of the present invention, in a case where the (A-3) and/or (A-6) above is “β₂ microglobulin”, the “β₂ microglobulin” comprises SEQ ID NO: 7 or a sequence having an amino acid sequence identity thereto of 80% or more, preferably 90% or more, more preferably 95% or more, still more preferably 98% or more, and further still more preferably 99% or more.

In an embodiment of the present invention, in a case where the (A-3) and/or (A-6) above is an “MHC class Iα chain”, the “MHC class Iα chain” comprises SEQ ID NO: 9 or a sequence having an amino acid sequence identity thereto of 80% or more, preferably 90% or more, more preferably 95% or more, still more preferably 98% or more, and further still more preferably 99% or more.

In an embodiment of the present invention, in a case where the (A-3) and/or (A-6) above is an “MHC class IIβ chain”, the “MHC class IIβ chain” comprises SEQ ID NO: 37 or a sequence having an amino acid sequence identity thereto of 80% or more, preferably 90% or more, more preferably 95% or more, still more preferably 98% or more, and further still more preferably 99% or more.

In an embodiment of the present invention, in a case where the (A-3) and/or (A-6) above is an “MHC class IIα chain”, the “MHC class IIα chain” comprises SEQ ID NO: 71 or a sequence having an amino acid sequence identity thereto of 80% or more, preferably 90% or more, more preferably 95% or more, still more preferably 98% or more, and further still more preferably 99% or more.

The “spacer sequence which may be present” of (A-2) and (A-4) in each of the embodiments may be independently selected when present. When (A-2) is present, for example, the spacer sequence may be, for example, a spacer sequence of SEQ ID NO: 5, 11, 29, 39, 77, or the like. When (A-4) is present, for example, the spacer sequence may be, for example, a spacer sequence of SEQ ID NO: 5, 11, 29, 39, 77, or the like.

In an embodiment of the present invention, the Tetraspanin of (A-5) in each of the embodiments is selected from the group consisting of CD9, CD63, and CD81. In an embodiment of the present invention, the Tetraspanin of (A-5) in each of the embodiments is CD81 (preferably, SEQ ID NO: 15 or the like or a sequence having an amino acid sequence identity thereto of 80% or more, preferably 90% or more, more preferably 95% or more, still more preferably 98% or more, and further still more preferably 99% or more).

In an embodiment of the present invention, the (A) above is
a fusion protein which comprises an amino acid sequence containing, from an N-terminal side thereof,
(A-1) an amino acid sequence of an MHC class I molecule-restricted antigen peptide,
(A-2) a spacer sequence of SEQ ID NO: 5,
(A-3) an amino acid sequence of a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order.

In an embodiment of the present invention, the (A) above is
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an amino acid sequence of an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more).

### Constitutional Requirement (B)

The “protein which comprises a first T-cell stimulatory cytokine and is capable of presenting the first T-cell stimulatory cytokine outside membrane” of the (B) above may comprise another protein or a domain thereof, or the like in addition to the first T-cell stimulatory cytokine as long as it is a protein capable of presenting the first T-cell stimulatory cytokine outside membrane.

In an embodiment of the present invention, the (B) above is a fusion protein which comprises a first T-cell stimulatory cytokine, and a membrane protein capable of being expressed in membrane of a cell or an extracellular vesicle or a transmembrane domain thereof or a protein capable of binding to membrane of an extracellular vesicle or a domain thereof, and is capable of presenting the first T-cell stimulatory cytokine outside membrane.

In an embodiment of the present invention, the (B) above is a fusion protein which comprises a first T-cell stimulatory cytokine and CD8 or a transmembrane domain thereof, the fusion protein being capable of presenting the first T-cell stimulatory cytokine outside membrane.

In an embodiment of the present invention, the (B) above is a fusion protein which comprises an amino acid sequence consisting of, from an N-terminal side thereof,
(B-3) a first T-cell stimulatory cytokine,
(B-4) a spacer sequence which may be optionally present, and
(B-5) CD8 or a transmembrane domain thereof,
the fusion protein being capable of presenting the first T-cell stimulatory cytokine outside the membrane.

In an embodiment of the present invention, the (B) above is
(B) a fusion protein which comprises a first T-cell stimulatory cytokine and a partial sequence of a Tetraspanin and is capable of presenting the first T-cell stimulatory cytokine outside membrane, in which the partial sequence of the Tetraspanin contains at least two transmembrane domains and the first T-cell stimulatory cytokine is disposed between the two transmembrane domains, or
(B) a fusion protein which comprises a first T-cell stimulatory cytokine and MFG-E8 or a domain thereof and is capable of presenting the first T-cell stimulatory cytokine.

Examples of the expression “the partial sequence of the Tetraspanin contains at least two transmembrane domains and the first T-cell stimulatory cytokine is disposed between the two transmembrane domains” used in the present specification include a case where the partial sequence of the Tetraspanin contains at least TM1 and TM2 of the Tetraspanin, and the first T-cell stimulatory cytokine is disposed between TM1 and TM2, and a case where the partial sequence of the Tetraspanin contains at least TM3 and TM4 of the Tetraspanin, and the first T-cell stimulatory cytokine is disposed between TM3 and TM4.

In an embodiment of the present invention, the (B) above is
a fusion protein which comprises an amino acid sequence consisting of, from an N-terminal side thereof,
(B-1) a partial sequence of a Tetraspanin containing, from an N-terminal side thereof, a transmembrane domain 1, a small extracellular loop, a transmembrane domain 2, a small intracellular loop, and a transmembrane domain 3,
(B-2) a spacer sequence which may be optionally present,
(B-3) a first T-cell stimulatory cytokine,
(B-4) a spacer sequence which may be optionally present, and
(B-5) a partial sequence of a Tetraspanin containing a transmembrane domain 4, in this order,
the fusion protein being capable of presenting the first T-cell stimulatory cytokine outside membrane, or
(B) a fusion protein which comprises an amino acid sequence consisting of, from an N-terminal side thereof,
(B-3) a first T-cell stimulatory cytokine,
(B-4) a spacer sequence which may be optionally present, and
(B-5) MFG-E8,
the fusion protein being capable of presenting the first T-cell stimulatory cytokine outside membrane.

As disclosed in WO 2016/139354 A, it has been reported that the Tetraspanin can be expressed in membranes even when a large extracellular loop (LEL) thereof is entirely or partially replaced by a different amino acid sequence. Therefore, the first T-cell stimulatory cytokine of (B-3) may be inserted in place of the LEL of the Tetraspanin or may be inserted at any site in the LEL of the Tetraspanin or a partial sequence thereof, by a spacer sequence which may be present.

The “partial sequence of the Tetraspanin containing a transmembrane domain 1, a small extracellular loop, a transmembrane domain 2, a small intracellular loop, and a transmembrane domain 3” of (B-1) usually does not contain a transmembrane domain 4 of the Tetraspanin. The “partial sequence of the Tetraspanin containing a transmembrane domain 1, a small extracellular loop, a transmembrane domain 2, a small intracellular loop, and a transmembrane domain 3” of (B-1) may contain a large extracellular loop or a partial sequence thereof. In (B-1), the transmembrane domain 1, the small extracellular loop, the transmembrane domain 2, the small intracellular loop, and the transmembrane domain 3 may be sequences derived from different Tetraspanins, respectively, or all the domains may be sequences derived from the same Tetraspanin. Preferably, in (B-1), all the transmembrane domain 1, the small extracellular loop, the transmembrane domain 2, the small intracellular loop, and the transmembrane domain 3 may be sequences derived from the same Tetraspanin.

In an embodiment of the present invention, in (B-1), all the partial sequences of the Tetraspanin containing a transmembrane domain 1, a small extracellular loop, a transmembrane domain 2, a small intracellular loop, and a transmembrane domain 3 are partial sequences derived from CD9, CD63, or CD81. In an embodiment of the present invention, all the partial sequences of the Tetraspanin containing a transmembrane domain 1, a small extracellular loop, a transmembrane domain 2, a small intracellular loop, and a transmembrane domain 3 of (B-1) are preferably partial sequences derived from CD63 or CD81 (preferably. SEQ ID NO: 57, SEQ ID NO: 61, or the like, or a sequence having an amino acid sequence identity thereto of 80% or more, preferably 90% or more, more preferably 95% or more, still more preferably 98% or more, and further still more preferably 99% or more).

The “partial sequence of the Tetraspanin containing a transmembrane domain 4” of (B-5) usually does not contain a transmembrane domain 1, a small extracellular loop, a transmembrane domain 2, a small intracellular loop, and a transmembrane domain 3 of the Tetraspanin. The “partial sequence of the Tetraspanin containing a transmembrane domain 4” of (B-5) may contain a large extracellular loop or a partial sequence thereof. The transmembrane domain 4 in (B-5) may be a sequence derived from a Tetraspanin different from that in (B-1), or may be a sequence derived from the same Tetraspanin as that in (B-1). Preferably, the transmembrane domain 4 in (B-5) is a sequence derived from the same Tetraspanin as that in (B-1). In an embodiment of the present invention, in (B-5), the partial sequence of the Tetraspanin containing a transmembrane domain 4 is a partial sequence derived from CD9, CD63, or CD81. In an embodiment of the present invention, the partial sequence of the Tetraspanin containing a transmembrane domain 4 of (B-5) is a partial sequence derived from CD63 or CD81 (preferably. SEQ ID NO: 59, SEQ ID NO: 63, or the like, or a sequence having an amino acid sequence identity thereto of 80% or more, preferably 90% or more, more preferably 95% or more, still more preferably 98% or more, and further still more preferably 99% or more).

In an embodiment of the present invention, the “partial sequence of the Tetraspanin containing a transmembrane domain 1, a small extracellular loop, a transmembrane domain 2, a small intracellular loop, and a transmembrane domain 3” of (B-1) is a partial sequence derived from CD63 (preferably. SEQ ID NO: 57 or the like or a sequence having an amino acid sequence identity thereto of 80% or more. 30) preferably 90% or more, more preferably 95% or more, still more preferably 98% or more, and further still more preferably 99% or more), and the “partial sequence of the Tetraspanin containing a transmembrane domain 4” of (B-5) is a partial sequence derived from CD63 (preferably. SEQ ID NO: 59 or the like or a sequence having an amino acid sequence identity thereto of 80% or more, preferably 90% or more, more preferably 95% or more, still more preferably 98% or more, and further still more preferably 99% or more). In an embodiment of the present invention, the “partial sequence of the Tetraspanin containing a transmembrane domain 1, a small extracellular loop, a transmembrane domain 2, a small intracellular loop, and a transmembrane domain 3” of (B-1) is a partial sequence derived from CD81 (preferably, SEQ ID NO: 61 or the like or a sequence having an amino acid sequence identity thereto of 80% or more, preferably 90% or more, more preferably 95% or more, still more preferably 98% or more, and further still more preferably 99% or more), and the “partial sequence of the Tetraspanin containing a transmembrane domain 4” of (B-5) is a partial sequence derived from CD81 (preferably, SEQ ID NO: 63 or the like or a sequence having an amino acid sequence identity thereto of 80% or more, preferably 90% or more, more preferably 95% or more, still more preferably 98% or more, and further still more preferably 99% or more).

The fusion protein of the (B) above is a fusion protein comprising a partial sequence of a Tetraspanin, and in a case where one or more of the (A) above and (C) present in some cases described below contain a fusion protein comprising an amino acid sequence of a Tetraspanin, the fusion protein of the (B) above may be a fusion protein different from the fusion protein of the (A) above and/or (C) present in some cases described below, or may constitute a part of the fusion protein of the (A) above and/or (C) present in some cases described below. The expression that the fusion protein of the (B) above “constitutes a part of the fusion protein of the (A) above and/or (C) present in some cases described below” includes, for example, a case where the Tetraspanin of (A-5) constitutes the fusion protein of (B), and/or a case where a Tetraspanin of (C-3) present in some cases described below is the fusion protein of (B).

The “MFG-E8” of the (B-5) above is preferably SEQ ID NO: 49 or the like or a sequence having an amino acid sequence identity thereto of 80% or more, preferably 90% or more, more preferably 95% or more, still more preferably 98% or more, and further still more preferably 99% or more.

In an embodiment of the present invention, in (B-3) of each of the embodiments, the first T-cell stimulatory cytokine is IL-2, IL-4, IL-6, IL-12, IL-15, or TGF-β. In an embodiment of the present invention, the first T-cell stimulatory cytokine in (B-3) in each of the embodiments is IL-2 (preferably, SEQ ID NO: 25 or the like or a sequence having an amino acid sequence identity thereto of 80% or more, preferably 90% or more, more preferably 95% or more, still more preferably 98% or more, and further still more preferably 99% or more), IL-4 (preferably, SEQ ID NO: 53 or the like or a sequence having an amino acid sequence identity thereto of 80% or more, preferably 90% or more, more preferably 95% or more, still more preferably 98% or more, and further still more preferably 99% or more), or TGF-β (preferably, SEQ ID NO: 73 or the like or a sequence having an amino acid sequence identity thereto of 80% or more, preferably 90% or more, more preferably 95% or more, still more preferably 98% or more, and further still more preferably 99% or more).

The “spacer sequence which may be present” in (B-2) and (B-4) in each of the embodiments may be independently selected when present. When (B-2) is present, for example, the spacer sequence may be, for example, a spacer sequence of SEQ ID NO: 5, 11, 29, 39, 77, or the like. When (B-4) is present, for example, the spacer sequence may be, for example, a spacer sequence of SEQ ID NO: 5, 11, 29, 39, 77, or the like.

In an embodiment of the present invention, the (B) above is
a fusion protein of which an amino acid sequence consists of, from an N-terminal side thereof,
(B-1) a partial sequence of a Tetraspanin of SEQ ID NO: 57 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) a first T-cell stimulatory cytokine which is IL-2 of SEQ ID NO: 25 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) a partial sequence of a Tetraspanin of SEQ ID NO: 59 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside membrane.

In an embodiment of the present invention, the (B) above is a fusion protein capable of presenting the first T-cell stimulatory cytokine of SEQ ID NO: 31 (or a sequence having an amino acid sequence identity thereto of 80% or more, preferably 90% or more, more preferably 95% or more, still more preferably 98% or more, and further still more preferably 99% or more), outside membrane.

In an embodiment of the present invention, the (B) above is
a fusion protein of which an amino acid sequence consists of, from an N-terminal side thereof,
(B-1) a partial sequence of a Tetraspanin of SEQ ID NO: 61 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) a first T-cell stimulatory cytokine which is IL-4 of SEQ ID NO: 53 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) a partial sequence of a Tetraspanin of SEQ ID NO: 63 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside membrane.

In an embodiment of the present invention, the (B) above is a fusion protein capable of presenting the first T-cell stimulatory cytokine of SEQ ID NO: 55 (or a sequence having an amino acid sequence identity thereto of 80% or more, preferably 90% or more, more preferably 95% or more, still more preferably 98% or more, and further still more preferably 99% or more), outside membrane.

In an embodiment of the present invention, the (B) above is
a fusion protein of which an amino acid sequence consists of, from an N-terminal side thereof,
(B-3) a first T-cell stimulatory cytokine which is TGF-β of SEQ ID NO: 73 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) MFG-E8 of SEQ ID NO: 49 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside membrane.

In an embodiment of the present invention, the (B) above is a fusion protein capable of presenting the first T-cell stimulatory cytokine of SEQ ID NO: 75 (or a sequence having an amino acid sequence identity thereto of 80% or more, preferably 90% or more, more preferably 95% or more, still more preferably 98% or more, and further still more preferably 99% or more), outside membrane.

### Second (or Higher) T-Cell Stimulatory Cytokines

The antigen-presenting extracellular vesicle described in the present specification may further contain second (or higher) T-cell stimulatory cytokines in addition to the first T-cell stimulatory cytokine. Therefore, in an embodiment of the present invention, the antigen-presenting cell or the antigen-presenting extracellular vesicle described in the present specification may further contain a second T-cell stimulatory cytokine. In particular, in a case where the MHC molecule capable of presenting an antigen is an MHC class II molecule capable of presenting an antigen, it is preferable that the antigen-presenting cell or the antigen-presenting extracellular vesicle described in the present specification contains a second T-cell stimulatory cytokine.

The second (or higher) T-cell stimulatory cytokines may be inserted into, for example, the (B) above (for example, the second (or higher) T-cell stimulatory cytokines may be linked to the N-terminus and/or the C-terminus of the “first T-cell stimulatory cytokine” of (B-3) by a spacer sequence or the like, if necessary). Alternatively, similar to the first T-cell stimulatory cytokine, the second (or higher) T-cell stimulatory cytokines may be contained in the membrane of the antigen-presenting cell or the antigen-presenting extracellular vesicle described in the present specification as a protein (or a fusion protein) different from the protein (or the fusion protein) of the constitutional requirement (B) described in the present specification by having the same configuration as that of the constitutional requirement (B) described in the present specification.

In an embodiment of the present invention, the second T-cell stimulatory cytokine is IL-2, IL-4, IL-6, IL-12, or TGF-β. In an embodiment of the present invention, the second T-cell stimulatory cytokine is TGF-β (preferably, SEQ ID NO: 73 or the like or a sequence having an amino acid sequence identity thereto of 80% or more, preferably 90% or more, more preferably 95% or more, still more preferably 98% or more, and further still more preferably 99% or more).

In an embodiment of the present invention, the first T-cell stimulatory cytokine is IL-2 or IL-4 (preferably, SEQ ID NO: 25, SEQ ID NO: 53, or the like or a sequence having an amino acid sequence identity thereto of 80% or more, preferably 90% or more, more preferably 95% or more, still more preferably 98% or more, and further still more preferably 99% or more), and the second T-cell stimulatory cytokine is TGF-β (preferably, SEQ ID NO: 73 or a sequence having an amino acid sequence identity thereto of 80% or more, preferably 90% or more, more preferably 95% or more, still more preferably 98% or more, and further still more preferably 99% or more).

In an embodiment of the present invention, the antigen-presenting cell or the antigen-presenting extracellular vesicle described in the present specification is an antigen-presenting cell an antigen-presenting extracellular vesicle, the membrane of which contains:
(A) a fusion protein or a protein complex which comprises an antigen-presenting MHC molecule, and a membrane protein capable of being expressed in membrane of a cell or an extracellular vesicle or a transmembrane domain thereof or a protein capable of binding to membrane of an extracellular vesicle or a domain thereof, and is capable of presenting the antigen outside membrane; and
(B) a fusion protein which comprises a first T-cell stimulatory cytokine, and a membrane protein capable of being expressed in membrane of a cell or an extracellular vesicle or a transmembrane domain thereof or a protein capable of binding to membrane of an extracellular vesicle or a domain thereof, and is capable of presenting the first T-cell stimulatory cytokine outside membrane.

In an embodiment of the present invention, the antigen-presenting cell described in the present specification is an antigen-presenting cell, the membrane of which contains:
(A) a protein complex capable of presenting an antigen peptide outside membrane, in which the protein complex contains
a fusion protein which comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of an MHC class Iα chain, in this order, and
a protein which comprises an amino acid sequence containing,
(A-6) an amino acid sequence of β₂ microglobulin; and
(B) a fusion protein which comprises an amino acid sequence containing, from an N-terminal side thereof,
(B-3) an amino acid sequence of a first T-cell stimulatory cytokine,
(B-4) a spacer sequence which may be optionally present, and
(B-5) an amino acid sequence of CD8 or a transmembrane domain thereof, in this order,
the fusion protein being capable of presenting the first T-cell stimulatory cytokine outside the membrane.

In an embodiment of the present invention, the antigen-presenting extracellular vesicle described in the present specification is an antigen-presenting extracellular vesicle the membrane of which contains:
(A) a fusion protein or a protein complex which contains an antigen-presenting MHC molecule and a Tetraspanin or a transmembrane domain thereof or MFG-E8 or a domain thereof, and is capable of presenting the antigen outside membrane; and
(B) a fusion protein which contains a first T-cell stimulatory cytokine and a partial sequence of a Tetraspanin, and is capable of presenting the first T-cell stimulatory cytokine outside membrane, in which the partial sequence of the Tetraspanin contains at least two transmembrane domains, and the first T-cell stimulatory cytokine is disposed between the two transmembrane domains, or
(B) a fusion protein which contains a first T-cell stimulatory cytokine and MFG-E8 or a domain thereof, and is capable of presenting the first T-cell stimulatory cytokine outside membrane.

In an embodiment of the present invention, the antigen-presenting extracellular vesicle described in the present specification is an antigen-presenting extracellular vesicle the membrane of which contains:
(A) a fusion protein capable of presenting an antigen peptide outside membrane, in which the fusion protein comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of a single chain MHC molecule,
(A-4) a spacer sequence which may be optionally present, and
(A-5) an amino acid sequence of a Tetraspanin or a transmembrane domain thereof, in this order, or
(A) a protein complex capable of presenting an antigen peptide outside membrane, in which the protein complex contains, a fusion protein which comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of an MHC class Iα chain, β₂ microglobulin, an MHC class IIα chain, or an MHC class IIβ chain,
(A-4) a spacer sequence which may be optionally present, and
(A-5) an amino acid sequence of a Tetraspanin or a transmembrane domain thereof, in this order, and
a protein which comprises
(A-6) an amino acid sequence of β₂ microglobulin, an MHC class Iα chain, an MHC class IIβ chain, or an MHC class IIα chain; and
(B) a fusion protein which comprises an amino acid sequence containing, from an N-terminal side thereof,
(B-1) a partial sequence of a Tetraspanin containing, from an N-terminal side thereof, a transmembrane domain 1, a small extracellular loop, a transmembrane domain 2, a small intracellular loop, and a transmembrane domain 3,
(B-2) a spacer sequence which may be optionally present,
(B-3) an amino acid sequence of a first T-cell stimulatory cytokine,
(B-4) a spacer sequence which may be optionally present, and
(B-5) a partial sequence of a Tetraspanin containing a transmembrane domain 4, in this order,
the fusion protein being capable of presenting the first T-cell stimulatory cytokine outside membrane, or
(B) a fusion protein which comprises an amino acid sequence containing, from an N-terminal side thereof,
(B-3) an amino acid sequence of a first T-cell stimulatory cytokine,
(B-4) a spacer sequence which may be optionally present, and
(B-5) an amino acid sequence of MFG-E8 or a membrane-binding domain thereof, in this order,
the fusion protein being capable of presenting the first T-cell stimulatory cytokine outside membrane.

In an embodiment of the present invention, the antigen-presenting extracellular vesicle described in the present specification is an antigen-presenting extracellular vesicle the membrane of which contains:
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of a single chain MHC class I molecule,
(A-4) a spacer sequence which may be optionally present, and
(A-5) an amino acid sequence of a Tetraspanin or a transmembrane domain thereof, in this order.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of an MHC class IIβ chain,
(A-4) a spacer sequence which may be optionally present, and
(A-5) an amino acid sequence of a Tetraspanin or a transmembrane domain thereof, in this order, and
a protein which comprises
(A-6) an amino acid sequence of an MHC class IIα chain.

In an embodiment of the present invention, the first T-cell stimulatory cytokine is IL-2, IL-4, IL-6, IL-12, IL-15, or TGF-β, and provides the antigen-presenting extracellular vesicle described in the present specification.

In an embodiment of the present invention, the antigen-presenting extracellular vesicle is the extracellular vesicle described in the present specification that further presents a T-cell costimulatory molecule outside membrane (exemplifying a model thereof in (2) of FIG. 2K).

Such an extracellular vesicle may present a T-cell costimulatory molecule outside membrane by containing a protein specified in the following (C) in membrane thereof.

Alternatively, a T-cell costimulatory molecule may be attached to membrane surface of an isolated extracellular vesicle later. An attachment method is not particularly limited, an antigen-presenting MHC molecule and a T-cell stimulatory cytokine may be attached to membrane surface by binding each phospholipid to a T-cell costimulatory molecule and incorporating a phospholipid moiety into membrane of an extracellular vesicle.

In an embodiment of the present invention, the antigen-presenting extracellular vesicle described in the present specification is an antigen-presenting extracellular vesicle the membrane of which contains:
(C) a protein which comprises a T-cell costimulatory molecule and is capable of allowing the T-cell costimulatory molecule to interact with T cells.

### Constitutional Requirement (C)

The “protein which comprises a T-cell costimulatory molecule and is capable of allowing the T-cell costimulatory molecule to interact with T cells” of the (C) above may contain another protein or a domain thereof, or the like in addition to the T-cell costimulatory molecule as long as it is a protein capable of allowing a T-cell costimulatory molecule to interact with T cells.

In an embodiment of the present invention, the (C) above is a fusion protein which comprises a T-cell costimulatory molecule, and a membrane protein capable of being expressed in membrane of a cell or an extracellular vesicle or a transmembrane domain thereof or a protein capable of binding to membrane of an extracellular vesicle or a domain thereof, and is capable of allowing the T-cell costimulatory molecule to interact with T cells.

In an embodiment of the present invention, the (C) above is a protein which comprises a T-cell costimulatory molecule containing a transmembrane domain, the protein being capable of allowing the T-cell costimulatory molecule to interact with T cells.

In an embodiment of the present invention, the (C) above is a fusion protein which comprises a T-cell costimulatory molecule, and a Tetraspanin or a transmembrane domain thereof or MFG-E8 or a domain thereof, and is capable of allowing the T-cell costimulatory molecule to interact with T cells.

In an embodiment of the present invention, the (C) above is
(C) a fusion protein which comprises an amino acid sequence containing, from an N-terminal side thereof,
(C-1) an amino acid sequence of a T-cell costimulatory molecule,
(C-2) a spacer sequence which may be optionally present, and
(C-3) an amino acid sequence of a Tetraspanin or a transmembrane domain thereof,
the fusion protein being capable of allowing the T-cell costimulatory molecule to interact with T cells.

In an embodiment of the present invention, the T-cell costimulatory molecule of (C-1) is CD80 or CD86. In an embodiment of the present invention, the T-cell costimulatory molecule in (C-1) is CD80 (preferably, SEQ ID NO: 67 or the like or a sequence having an amino acid sequence identity thereto of 80% or more, preferably 90% or more, more preferably 95% or more, still more preferably 98% or more, and further still more preferably 99% or more).

The “spacer sequence which may be present” of (C-2) may be, for example, a spacer sequence of SEQ ID NO: 5, 11, 29, 39, 77, or the like when present.

In an embodiment of the present invention, the Tetraspanin of (C-3) is selected from the group consisting of CD9, CD63, and CD81. In an embodiment of the present invention, the Tetraspanin in (C-3) is CD9 (preferably, SEQ ID NO: 21 or the like or a sequence having an amino acid sequence identity thereto of 80% or more, preferably 90% or more, more preferably 95% or more, still more preferably 98% or more, and further still more preferably 99% or more).

In an embodiment of the present invention, the (C) above is
(C) a fusion protein which comprises an amino acid sequence containing, from an N-terminal side thereof,
(C-1) an amino acid sequence of a T-cell costimulatory molecule that is CD80 of SEQ ID NO: 67 (or a sequence having an amino acid sequence identity thereto of 80% or more, preferably 90% or more, more preferably 95% or more, still more preferably 98% or more, and further still more preferably 99% or more), and
(C-3) an amino acid sequence of a Tetraspanin or a transmembrane domain thereof of SEQ ID NO: 21 (or a sequence having an amino acid sequence identity thereto of 80% or more, preferably 90% or more, more preferably 95% or more, still more preferably 98% or more, and further still more preferably 99% or more), in this order,
the fusion protein being capable of allowing the T-cell costimulatory molecule to interact with T cells.

In an embodiment of the present invention, the (C) above is a fusion protein capable of allowing the T-cell costimulatory molecule of SEQ ID NO: 69 (or a sequence having an amino acid sequence identity thereto of 80% or more, preferably 90% or more, more preferably 95% or more, still more preferably 98% or more, and further still more preferably 99% or more), to interact with T cells.

In an embodiment of the present invention, the antigen-presenting extracellular vesicle described in the present specification is an antigen-presenting extracellular vesicle the membrane of which contains:
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence of SEQ ID NO: 5,
(A-3) an amino acid sequence of a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(B) a fusion protein which comprises an amino acid sequence containing, from an N-terminal side thereof,
(B-1) a partial sequence of a Tetraspanin of SEQ ID NO: 57 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) a first T-cell stimulatory cytokine which is IL-2 of SEQ ID NO: 25 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) a partial sequence of a Tetraspanin of SEQ ID NO: 59 (or a sequence having an amino acid sequence identity thereto of 80% or more, preferably 90% or more, more preferably 95% or more, still more preferably 98% or more, and further still more preferably 99% or more), in this order,
the fusion protein being capable of allowing the first T-cell stimulatory cytokine to interact with T cells.

In an embodiment of the present invention, the antigen-presenting extracellular vesicle described in the present specification is an antigen-presenting extracellular vesicle the membrane of which contains:
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence of SEQ ID NO: 5,
(A-3) an amino acid sequence of a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(B) a fusion protein of SEQ ID NO: 31 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside the membrane.

In an embodiment of the present invention, the antigen-presenting extracellular vesicle described in the present specification is an antigen-presenting extracellular vesicle the membrane of which contains:
(A) a fusion protein capable of presenting an antigen peptide outside membrane, in which the fusion protein comprises an amino acid sequence consisting of, from an N-terminal side thereof,
(A-1) an MHC class I molecule-restricted antigen peptide,
(A-2) a spacer sequence of SEQ ID NO: 5,
(A-3) a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) a Tetraspanin of SEQ ID NO: 15 (or a sequence having an amino acid sequence identity thereto of 80% or more, preferably 90% or more, more preferably 95% or more, still more preferably 98% or more, and further still more preferably 99% or more), in this order;
(B) a fusion protein of which an amino acid sequence consists of, from an N-terminal side thereof,
(B-1) a partial sequence of a Tetraspanin of SEQ ID NO: 57 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) a partial sequence of a first T-cell stimulatory cytokine, which is IL-2 of SEQ ID NO: 25 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29; and
(B-5) a partial sequence of a Tetraspanin of SEQ ID NO: 59 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside the membrane; and
(C) a fusion protein of which an amino acid sequence consists of, from an N-terminal side thereof,
(C-1) a T-cell costimulatory molecule which is CD80 of SEQ ID NO: 67 (or a sequence having an amino acid sequence identity thereto of 80% or more, preferably 90% or more, more preferably 95% or more, still more preferably 98% or more, and further still more preferably 99% or more), and
(C-3) a Tetraspanin of SEQ ID NO: 21 (or a sequence having an amino acid sequence identity thereto of 80% or more, preferably 90% or more, more preferably 95% or more, still more preferably 98% or more, and further still more preferably 99% or more),
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence of SEQ ID NO: 5,
(A-3) an amino acid sequence of a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order;
(B) a fusion protein of SEQ ID NO: 31 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside the membrane; and
(C) a fusion protein of SEQ ID NO: 69 (or a sequence having an amino acid sequence identity thereto of 80% or more, preferably 90% or more, more preferably 95% or more, still more preferably 98% or more, and further still more preferably 99% or more),
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex consists of:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more);
(B) a fusion protein which comprises an amino acid sequence containing, from an N-terminal side thereof,
(B-1) a partial sequence of a Tetraspanin of SEQ ID NO: 57 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) an amino acid sequence of a first T-cell stimulatory cytokine which is IL-2 of SEQ ID NO: 25 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29; and
(B-5) a partial sequence of a Tetraspanin of SEQ ID NO: 59 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the first T-cell stimulatory cytokine outside the membrane; and
(C) a fusion protein which comprises an amino acid sequence containing, from an N-terminal side thereof,
(C-1) an amino acid sequence of a T-cell costimulatory molecule which is CD80 of SEQ ID NO: 67 (or a sequence having an amino acid sequence identity thereto of 80% or more, preferably 90% or more, more preferably 95% or more, still more preferably 98% or more, and further still more preferably 99% or more), and
(C-3) an amino acid sequence of a Tetraspanin or a transmembrane domain thereof of SEQ ID NO: 21 (or a sequence having an amino acid sequence identity thereto of 80% or more, preferably 90% or more, more preferably 95% or more, still more preferably 98% or more, and further still more preferably 99% or more), in this order,
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex consists of:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more);
(B) an amino acid sequence of a fusion protein of SEQ ID NO: 31 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside the membrane; and
(C) an amino acid sequence of a fusion protein of SEQ ID NO: 69 (or a sequence having an amino acid sequence identity thereto of 80% or more, preferably 90% or more, more preferably 95% or more, still more preferably 98% or more, and further still more preferably 99% or more),
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex consists of:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more);
(B) a fusion protein which comprises an amino acid sequence containing, from an N-terminal side thereof,
(B-1) a partial sequence of a Tetraspanin of SEQ ID NO: 57 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) an amino acid sequence of a first T-cell stimulatory cytokine which is IL-2 of SEQ ID NO: 25 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29; and
(B-5) a partial sequence of a Tetraspanin of SEQ ID NO: 59 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the first T-cell stimulatory cytokine outside the membrane:
(B′) a fusion protein which comprises an amino acid sequence containing, from an N-terminal side thereof,
(B-3) an amino acid sequence of a second T-cell stimulatory cytokine which is TGF-β of SEQ ID NO: 73 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) an amino acid sequence of MFG-E8 or a membrane-binding domain thereof of SEQ ID NO: 49 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the second T-cell stimulatory cytokine outside the membrane; and
(C) a fusion protein which comprises an amino acid sequence containing, from an N-terminal side thereof,
(C-1) an amino acid sequence of a T-cell costimulatory molecule which is CD80 of SEQ ID NO: 67 (or a sequence having an amino acid sequence identity thereto of 80% or more, preferably 90% or more, more preferably 95% or more, still more preferably 98% or more, and further still more preferably 99% or more), and
(C-3) an amino acid sequence of a Tetraspanin or a transmembrane domain thereof of SEQ ID NO: 21 (or a sequence having an amino acid sequence identity thereto of 80% or more, preferably 90% or more, more preferably 95% or more, still more preferably 98% or more, and further still more preferably 99% or more), in this order,
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex consists of:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a membrane-binding domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more);
(B) a fusion protein of SEQ ID NO: 31 (or a sequence having an amino acid sequence identity thereto of 80% or more, preferably 90% or more, more preferably 95% or more, still more preferably 98% or more, and further still more preferably 99%
the fusion protein being capable of presenting the first T-cell stimulatory cytokine outside the membrane; or more),
(B′) a fusion protein of SEQ ID NO: 75 (or a sequence having an amino acid sequence identity thereto of 80% or more, preferably 90% or more, more preferably 95% or more, still more preferably 98% or more, and further still more preferably 99% or more),
fusion protein being capable of presenting the second T-cell stimulatory cytokine outside the membrane; and
(C) an amino acid sequence of a fusion protein of SEQ ID NO: 69 (or a sequence having an amino acid sequence identity thereto of 80% or more, preferably 90% or more, more preferably 95% or more, still more preferably 98% or more, and further still more preferably 99% or more),
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex consists of:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more);
(B) a fusion protein which comprises an amino acid sequence containing, from an N-terminal side thereof,
(B-1) a partial sequence of a Tetraspanin of SEQ ID NO: 61 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) an amino acid sequence of a first T-cell stimulatory cytokine which is IL-4 of SEQ ID NO: 53 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29; and
(B-5) a partial sequence of a Tetraspanin of SEQ ID NO: 63 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the first T-cell stimulatory cytokine outside the membrane; and
(C) a fusion protein which comprises an amino acid sequence containing, from an N-terminal side thereof,
(C-1) an amino acid sequence of a T-cell costimulatory molecule which is CD80 of SEQ ID NO: 67 (or a sequence having an amino acid sequence identity thereto of 80% or more, preferably 90% or more, more preferably 95% or more, still more preferably 98% or more, and further still more preferably 99% or more), and
(C-3) an amino acid sequence of a Tetraspanin or a transmembrane domain thereof of SEQ ID NO: 21 (or a sequence having an amino acid sequence identity thereto of 80% or more, preferably 90% or more, more preferably 95% or more, still more preferably 98% or more, and further still more preferably 99% or more), in this order,
the fusion protein being capable of allowing the T-cell costimulatory molecule to interact with T cells.

In an embodiment of the present invention, the antigen-presenting extracellular vesicle described in the present specification is an antigen-presenting extracellular vesicle the membrane of which contains:
(A) a protein complex capable of presenting an antigen peptide outside membrane, in which the protein complex consists of:
a fusion protein which comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order; and
(A-6) an MHC class IIα chain of SEQ ID NO: 71 (or a sequence having an amino acid sequence identity thereto of 80% or more, preferably 90% or more, more preferably 95% or more, still more preferably 98% or more, and further still more preferably 99% or more);
(B) a fusion protein of SEQ ID NO: 55 (or a sequence having an amino acid sequence identity thereto of 80% or more, preferably 90% or more, more preferably 95% or more, still more preferably 98% or more, and further still more preferably 99% or more),
the fusion protein being capable of presenting the first T-cell stimulatory cytokine outside the membrane; and
(C) a fusion protein of SEQ ID NO: 69 (or a sequence having an amino acid sequence identity thereto of 80% or more, preferably 90% or more, more preferably 95% or more, still more preferably 98% or more, and further still more preferably 99% or more),
the fusion protein being capable of allowing the T-cell costimulatory molecule to interact with T cells.

In an embodiment of the present invention, as for the (A), (B), and (C) above, (A) and (B) may be fused to form one molecule, (B) and (C) may be fused to form one molecule, and (A), (B), and (C) are fused to form one molecule. Such a fusion molecule may be translated as one protein molecule with or without a spacer sequence between (A), (B), and (C), or the proteins of (A), (B), and (C) may be fused by chemical crosslinking (for example, a disulfide bond between cysteine residues) to form one molecule.

Alternatively, the (A), (B), and (C) above may be functionally fused by sharing an element for localizing the proteins thereof in the cell or the extracellular vesicle, that is, a site of a “membrane protein capable of being expressed in membrane of a cell or an extracellular vesicle or a transmembrane domain thereof” or a “protein capable of binding to membrane of a cell or an extracellular vesicle or a domain thereof”.

For example, in an embodiment of the present invention,
the antigen-presenting extracellular vesicle may also contain (D) a fusion protein comprising:
(1) an antigen-presenting MHC molecule;
(2) at least one T-cell stimulatory cytokine; and
(3) a “membrane protein capable of being expressed in membrane of a cell or an extracellular vesicle or a transmembrane domain thereof” or a “protein capable of binding to membrane of a cell or an extracellular vesicle or a domain thereof”, the fusion protein being fused in a form of sharing the site of the “membrane protein capable of being expressed in membrane of an extracellular vesicle or the transmembrane domain thereof” or the “protein capable of binding to membrane of an extracellular vesicle or the domain thereof” in (A) and (B);
a fusion protein (F) comprising:
(1) an antigen-presenting MHC molecule;
(2) a T-cell costimulatory molecule; and
(3) a “membrane protein capable of being expressed in membrane of a cell or an extracellular vesicle or a transmembrane domain thereof” or a “protein capable of binding to membrane of an extracellular vesicle or a domain thereof”,
the fusion protein being fused in a form of sharing the site of the “membrane protein capable of being expressed in membrane of a cell or an extracellular vesicle or the transmembrane domain thereof” or the “protein capable of binding to membrane of an extracellular vesicle or the domain thereof” in (A) and (C);
a fusion protein (G) comprising:
(1) at least one T-cell stimulatory cytokine;
(2) a T-cell costimulatory molecule; and
(3) a “membrane protein capable of being expressed in membrane of a cell or an extracellular vesicle or a transmembrane domain thereof” or a “protein capable of binding to membrane of a cell or an extracellular vesicle or a domain thereof”,
the fusion protein being fused in a form of sharing the site of the “membrane protein capable of being expressed in membrane of an extracellular vesicle or the transmembrane domain thereof” or the “protein capable of binding to membrane of an extracellular vesicle or the domain thereof” in (B) and (C); or
a fusion protein (E) comprising:
(1) an antigen-presenting MHC molecule;
(2) at least one T-cell stimulatory cytokine;
(3) a T-cell costimulatory molecule; and
(4) a “membrane protein capable of being expressed in membrane of a cell or an extracellular vesicle or a transmembrane domain thereof” or a “protein capable of binding to membrane of an extracellular vesicle or a domain thereof”,
the fusion protein being fused in a form of sharing the site of the “membrane protein capable of being expressed in membrane of a cell or an extracellular vesicle or the transmembrane domain thereof” or the “protein capable of binding to membrane of an extracellular vesicle or the domain thereof” in (A) to (C).

In an embodiment of the present invention, the antigen-presenting extracellular vesicle may be an antigen-presenting extracellular vesicle containing a fusion protein (D) having the functions of the constitutional requirement (A) and the constitutional requirement (B) using the “protein which comprises a first T-cell stimulatory cytokine and is capable of presenting the first T-cell stimulatory cytokine outside membrane” of the constitutional requirement (B), instead of the “membrane protein capable of being expressed in membrane of a cell or an extracellular vesicle or the transmembrane domain thereof or the protein capable of binding to membrane of a cell or an extracellular vesicle” of the constitutional requirement (A).

Such a fusion protein (D) having the functions of the constitutional requirement (A) and the constitutional requirement (B) may be
(D) a fusion protein which comprises an antigen-presenting MHC molecule and at least one T-cell stimulatory cytokine and is capable of presenting the antigen and the T-cell stimulatory cytokine outside membrane.

The fusion protein may comprise the antigen-presenting MHC molecule, the at least one T-cell stimulatory cytokine, and a membrane protein capable of being localized to membrane of a cell or an extracellular vesicle or a transmembrane domain thereof or a protein capable of binding to membrane of a cell or an extracellular vesicle or a membrane-binding domain thereof.

In the fusion protein (D) having the functions of the constitutional requirement (A) and the constitutional requirement (B), the membrane protein capable of being localized to membrane of a cell or the protein capable of binding to membrane of a cell may be CD8, and an MHC molecule containing a transmembrane domain may perform this function.

The fusion protein may also comprise an amino acid sequence encoding, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule,
(D-4) a spacer sequence which may be optionally present, and
(D-5) a fusion peptide containing CD8 or a transmembrane domain thereof and the at least one T-cell stimulatory cytokine, in this order.

The fusion protein may also comprise an amino acid sequence encoding, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule containing a transmembrane domain,
(D-4) a spacer sequence which may be optionally present, and
(D-5) at least one T-cell stimulatory cytokine, in this order.

In the fusion protein (D) having the functions of the constitutional requirement (A) and the constitutional requirement (B), the membrane protein capable of being localized to membrane of an extracellular vesicle or the protein capable of binding to membrane of an extracellular vesicle may be a Tetraspanin or MFG-E8.

The fusion protein may also comprise an amino acid sequence encoding, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule,
(D-4) a spacer sequence which may be optionally present, and
(D-5) a fusion peptide comprising a Tetraspanin or a transmembrane domain thereof or MFG-E8 or a transmembrane domain thereof, and the at least one T-cell stimulatory cytokine, in this order.

The fusion protein may also comprise an amino acid sequence encoding, from an N-terminal side thereof,
(D-1) a fusion peptide comprising a Tetraspanin or a transmembrane domain thereof or MFG-E8 or a transmembrane domain thereof, and the at least one T-cell stimulatory cytokine,
(D-2) a spacer sequence which may be optionally present,
(D-3) an amino acid sequence of a single chain MHC molecule,
(D-4) a spacer sequence which may be optionally present, and
(D-5) an MHC molecule-restricted antigen peptide, in this order.

Here, the fusion peptide may also comprise an amino acid sequence encoding, from an N-terminal side thereof,
(1) a partial sequence of a Tetraspanin containing a transmembrane domain 1, a small intracellular loop, a transmembrane domain 2, a small extracellular loop, and a transmembrane domain 3,
(2) a spacer sequence which may be optionally present,
(3) an amino acid sequence of the at least one T-cell stimulatory cytokine,
(4) a spacer sequence which may be optionally present, and
(5) a partial sequence of a Tetraspanin containing a transmembrane domain 4, in this order.

The fusion peptide may also comprise an amino acid sequence encoding, from an N-terminal side thereof,
(1) an amino acid sequence of the at least one T-cell stimulatory cytokine,
(2) a spacer sequence which may be optionally present, and
(3) an amino acid sequence of MFG-E8 or a membrane-binding domain thereof, in this order.

In an embodiment of the present invention, the MHC molecule-restricted antigen peptide is an MHC class I molecule-restricted antigen peptide, the single chain MHC molecule may contain an extracellular domain of an MHC class Iα chain, the MHC molecule-restricted antigen peptide is an MHC class II molecule-restricted antigen peptide, and the single chain MHC molecule may contain an extracellular domain of an MHC class IIα chain and/or an extracellular domain of an MHC class IIβ chain.

In the aspect containing the fusion protein (D) having the functions of the constitutional requirement (A) and the constitutional requirement (B):
(C) a protein which comprises at least one T-cell costimulatory molecule and is capable of allowing the T-cell costimulatory molecule to interact with T cells may be further contained in the membrane:
the protein capable of interacting with T cells may also comprise the at least one T-cell costimulatory molecule, and a membrane protein capable of being expressed in membrane of a cell or an extracellular vesicle or a transmembrane domain thereof or a protein capable of binding to membrane of a cell or an extracellular vesicle or a domain thereof; and
the protein capable of interacting with T cells may also comprise the at least one T-cell costimulatory molecule, and a Tetraspanin or a transmembrane domain thereof or MFG-E8 or a domain thereof.

The protein capable of interacting with T cells may contain one T-cell costimulatory molecule containing a transmembrane domain.

In an embodiment of the present invention, the extracellular vesicle is an exosome.

The antigen-presenting cell or the antigen-presenting extracellular vesicle in the present specification may contain or be bound to a substance that may be therapeutically beneficial (for example, a low-molecular compound, a nucleic acid, or the like) inside the membrane thereof or in the membrane. Examples of a method for encapsulating the substance inside the membrane of the cell or the extracellular vesicle include, but are not limited to, a method in which the substance and the cell or the extracellular vesicle described in the present specification are mixed in a suitable solvent.

In an embodiment of the present invention, the antigen-presenting cell or the antigen-presenting extracellular vesicle may contain any protein preparation. The protein preparation is not particularly limited, but may be a protein that can also exist in nature such as erythropoietin, a synthetic protein that does not exist in nature such as an immunoglobulin-CTLA4 fusion protein, or a monoclonal antibody or an active fragment thereof. These protein preparations are fusion proteins with a membrane protein capable of being localized to membrane of a cell or an extracellular vesicle or a transmembrane domain thereof or a protein capable of binding to membrane of a cell or an extracellular vesicle or a membrane-binding domain thereof, and may be localized on the surface of the antigen-presenting extracellular vesicle. Such an antigen-presenting cell or an antigen-presenting extracellular vesicle 1) can be prepared by transfecting any cell with a vector for expressing a fusion protein; and 2) the antigen-presenting extracellular vesicle can be secreted by transfecting cells that produce extracellular vesicles.

Each fusion protein or protein complex or a protein preparation contained in the membrane of the antigen-presenting cell or the antigen-presenting extracellular vesicle described in the present specification may comprise one or a plurality of detectable labels. For example, the fusion protein or the protein complex or the protein preparation may be labeled with a specific lipoprotein molecule, a fluorophore, a radioactive material, or an enzyme (for example, peroxidase or phosphatase), or the like by a conventional method. These labels may be linked to the N-terminus or the C-terminus of the fusion protein or the protein complex or the protein preparation, for example, as a constituent element of the fusion protein or the protein complex or the protein preparation.

### Polynucleotide

In an embodiment of the present invention, there is provided a polynucleotide encoding each fusion protein or protein complex in (A) and (B), and (C) present in some cases that are contained in the membrane of the antigen-presenting cell or the antigen-presenting extracellular vesicle described in the present specification. In an embodiment of the present invention, there is provided a polynucleotide encoding each fusion protein or protein complex in (A) to (G) defined in the present specification.

In an embodiment of the present invention,
there is provided a polynucleotide comprising at least one sequence selected from the group consisting of:
(a) a sequence encoding a fusion protein (A) which comprises an antigen-presenting MHC molecule and is capable of presenting the antigen-presenting MHC molecule outside membrane of a cell or an extracellular vesicle:
(b) a sequence encoding a fusion protein (B) which comprises at least one T-cell stimulatory cytokine or subunit thereof and is capable of presenting the T-cell stimulatory cytokine outside membrane of a cell or an extracellular vesicle:
(c) a sequence encoding a fusion protein (C) which comprises a T-cell costimulatory molecule and is capable of presenting the T-cell costimulatory molecule outside membrane of a cell or an extracellular vesicle:
(d) a sequence encoding a fusion protein (D) which comprises an antigen-presenting MHC molecule and at least one T-cell stimulatory cytokine or subunit thereof and is capable of presenting the antigen and the T-cell stimulatory cytokine outside membrane of a cell or an extracellular vesicle; and
(e) a sequence encoding a fusion protein (E) which comprises an antigen-presenting MHC molecule, at least one T-cell stimulatory cytokine or subunit thereof, and a T-cell costimulatory molecule, and is capable of presenting the antigen, the T-cell stimulatory cytokine, and the T-cell costimulatory molecule outside membrane of a cell or an extracellular vesicle.

The sequences (a) to (e) include the sequences specifically described in the present specification and a sequence having high homology (homology of preferably 90% or more, more preferably 95% or more, and still more preferably 99% or more), but are not particularly limited thereto. Paralogs (i.e., gene sequences generated by gene duplication) or orthologs (i.e., groups of genes having homologous functions that exist in different organisms) may be used as long as they have the same functions, and sequences having modified (prohibited, deleted, substituted, or the like) sequence information are also included.

The “polynucleotide” used in the present specification means a single-stranded or double-stranded DNA molecule, an RNA molecule, a DNA-RNA chimeric molecule, or the like. The polynucleotide includes genomic DNA, cDNA, hnRNA, mRNA, and the like, and all naturally occurring or artificially modified derivatives thereof. The polynucleotide may be linear or cyclic.

The polynucleotide encoding each fusion protein or protein complex in (A) to (G) described above can be appropriately determined by those skilled in the art with reference to the amino acid sequence of the fusion protein or protein complex. Note that the amino acid sequence of each fusion protein or protein complex in (A) to (G) can be appropriately determined with reference to the amino acid sequence of each constituent element (for example, in the case of (A), (A-1) to (A-5), and (A-6) in some cases) in each fusion protein or protein complex. Any type of codon can be selected for use in determining a polynucleotide. For example, a polynucleotide may be determined in consideration of a frequency or the like of codons of cells to be transformed using a vector comprising the polynucleotide.

To the N-terminus of the polynucleotide encoding each fusion protein or protein complex in (A) to (G) described above, a polynucleotide encoding a signal peptide (signal sequence) may be added, if necessary.

As an amino acid sequence of the signal peptide, any amino acid sequence can be used, and for example, the amino acid sequence of the signal peptide may be determined in consideration of an amino acid sequence of a fusion protein to be expressed, and the like. Examples of the polynucleotide encoding a signal peptide include a polynucleotide (for example, SEQ ID NO: 2) encoding a signal peptide (for example, SEQ ID NO: 1) of β₂ microglobulin, a polynucleotide encoding a signal peptide of an MHC class Iα chain, a polynucleotide encoding a signal peptide of an MHC class IIα chain, and a polynucleotide (for example, SEQ ID NO: 34) encoding a signal peptide (for example, SEQ ID NO: 33) of an MHC class IIβ chain.

Information on each constituent element (for example, in the case of (A), (A-1) to (A-5), and (A-6) in some cases) of each fusion protein or protein complex in (A) to (G) described above, the amino acid sequence such as a signal peptide, and the polynucleotide encoding them may be appropriately obtained by searching, for example, a database of known literatures, NCBI (http://www.ncbi.nlm.nih.gov/guide/), and the like. In addition, for the amino acid sequence in the partial sequence of the Tetraspanin (for example, the partial sequences in (C-1) and (C-5)) and the polynucleotide encoding the amino acid sequence, WO 2016/139354 A may be referred to.

In an embodiment of the present invention, (a) may include the following:
a sequence encoding
(A) a fusion protein capable of presenting an antigen peptide outside membrane, in which the fusion protein comprises an amino acid sequence containing, from an N-terminal side thereof,
(A-1) an amino acid sequence of an MHC molecule-restricted antigen peptide,
(A-2) a spacer sequence which may be optionally present, and
(A-3) an amino acid sequence of a single chain MHC molecule containing a transmembrane domain, in this order.

In an embodiment of the present invention, (a) may include the following:
a sequence encoding
(A) a protein complex capable of presenting an antigen peptide, in which the protein complex contains,
a fusion protein which comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence consisting of an MHC class Iα chain, a fusion protein of β₂ microglobulin and an MHC class Iα chain, an MHC class IIα chain, or an MHC class IIβ chain, in this order, and
a protein which comprises
(A-6) an amino acid sequence of β₂ microglobulin, an MHC class Iα chain, an MHC class IIβ chain, or an MHC class IIα chain,
in which when translated here, (A-3) and (A-6) are preferably paired to form an MHC class I molecule or an MHC class II molecule.

In addition, in an embodiment of the present invention, (a) may contain a sequence in which the amino acid sequence consisting of the (A-1) to (A-3) and the sequence of (A-6) encode one fusion protein through the following 2A peptide sequence of:
at least one of

T2A:
(SEQ ID NO: 211)
(GSG)EGRGSLLTCGDVEENPGP,
P2A:
(SEQ ID NO: 212)
(GSG)ATNFSLLKQAGDVEENPGP,
E2A
(SEQ ID NO: 213)
(GSG)QCTNYALLKLAGDVESNPGP,
and
F2A
(SEQ ID NO: 214)
(GSG)VKQTLNFDLLKLAGDVESNPGP.

It is preferable that the 2A peptide sequence causes ribosome skipping, and in a case where the sequence encoding a fusion protein is actually translated, a fusion protein which comprises an amino acid sequence consisting of independent (A-1) to (A-3) and a protein containing an independent the sequence of (A-6) are translated, and the two translated proteins form an MHC class I molecule or an MHC class II molecule.

In an embodiment of the present invention, (a) may contain a sequence encoding:
(A) a fusion protein capable of presenting an antigen peptide outside membrane, in which the fusion comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of a single chain MHC class I molecule containing a transmembrane domain, in this order,
the fusion protein being capable of presenting the antigen peptide outside the membrane; and
(A) a fusion protein capable of presenting an antigen peptide outside membrane, in which the fusion comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of a single chain MHC class II molecule containing a transmembrane domain, in this order,
the fusion protein being capable of presenting the antigen peptide outside the membrane.

In an embodiment of the present invention, the (a) above may contain
(A) a protein complex capable of presenting an antigen peptide outside membrane, in which the protein complex comprises an amino acid sequence containing, an N-terminal side thereof,
(A-1) an amino acid sequence of an MHC class I molecule-restricted antigen peptide,
(A-2) a spacer sequence which may be optionally present, and
(A-3) an amino acid sequence of a fusion protein of β₂ microglobulin and an MHC class Iα chain, in this order.

In an embodiment of the present invention, the (a) above may contain
(A) a sequence encoding a protein complex capable of presenting an antigen peptide outside membrane, in which the protein complex contains a fusion protein which comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of an MHC class Iα chain, in this order, and
a fusion protein which comprises
(A-6) an amino acid sequence of β₂ microglobulin, and
even when the amino acid sequence consisting of (A-1) to (A-3) and the amino acid sequence of (A-6) may contain a sequence encoding one fusion protein through at least one 2A peptide sequence.

In an embodiment of the present invention, the (a) above may contain
(A) a sequence encoding a protein complex capable of presenting an antigen peptide outside membrane, in which the protein complex contains a fusion protein which comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of an MHC class IIβ chain, in this order, and a fusion protein which comprises
(A-6) an amino acid sequence of an MHC class IIα chain, and even when the amino acid sequence consisting of (A-1) to (A-3) and the amino acid sequence of (A-6) may contain a sequence encoding one fusion protein through at least one 2A peptide sequence.

In addition, in an embodiment of the present invention, the (a) above is
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of an MHC class IIα chain, in this order, and
a protein which comprises
(A-6) an amino acid sequence of an MHC class IIβ chain.

In an embodiment of the present invention, the (A) above is a fusion protein or a protein complex which comprises an antigen-presenting MHC molecule, and a Tetraspanin or a transmembrane domain thereof or MFG-E8 or a domain thereof, and is capable of presenting the antigen outside membrane.

In addition, in an embodiment of the present invention, the (a) above may contain:
(A) a sequence encoding a fusion protein capable of presenting an antigen peptide outside membrane, in which the fusion protein comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of a single chain MHC molecule,
(A-4) a spacer sequence which may be optionally present, and
(A-5) an amino acid sequence of a Tetraspanin or a transmembrane domain thereof, in this order; or
(A) a protein complex capable of presenting an antigen peptide outside membrane, in which the protein complex contains
a fusion protein which comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of an MHC class Iα chain, β₂ microglobulin, an MHC class IIα chain, or an MHC class IIβ chain,
(A-4) a spacer sequence which may be optionally present, and
(A-5) an amino acid sequence of a Tetraspanin or a transmembrane domain thereof, in this order, and
a protein which comprises
(A-6) an amino acid sequence of β₂ microglobulin, an MHC class Iα chain, an MHC class IIβ chain, or an MHC class IIα chain.

In an embodiment of the present invention, the (a) above may contain a sequence encoding
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of a single chain MHC class I molecule,
(A-4) a spacer sequence which may be optionally present, and
(A-5) an amino acid sequence of a Tetraspanin or a transmembrane domain thereof, in this order,
the fusion protein being capable of presenting the antigen peptide outside the membrane.

In addition, in an embodiment of the present invention, the (a) above may contain a sequence encoding
(A) a fusion protein capable of presenting an antigen peptide outside membrane, in which the fusion protein comprises an amino acid sequence consisting of, from an N-terminal side thereof,
(A-1) an amino acid sequence of an MHC class II molecule-restricted antigen peptide,
(A-2) a spacer sequence which may be optionally present,
(A-3) an amino acid sequence of a single chain MHC class II molecule,
(A-4) a spacer sequence which may be optionally present, and
(A-5) an amino acid sequence of a Tetraspanin or a transmembrane domain thereof, in this order,
the fusion protein being capable of presenting the antigen peptide outside the membrane.

In an embodiment of the present invention, the (a) above may include the following:
(A) a sequence encoding a fusion protein capable of presenting an antigen peptide outside membrane, in which the fusion protein comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of an MHC class Iα chain, β₂ microglobulin, an MHC class IIα chain, or an MHC class IIβ chain,
(A-4) a spacer sequence which may be optionally present, and
(A-5) an amino acid sequence of a Tetraspanin or a transmembrane domain thereof, in this order; and
may further contain a sequence encoding
(A-6) an amino acid sequence of β₂ microglobulin, an MHC class Iα chain, an MHC class IIβ chain, or an MHC class IIα chain.

When translated here, (A-3) and (A-6) are preferably paired to form an MHC class I molecule or an MHC class II molecule.

In addition, in an embodiment of the present invention, the (a) above may contain a sequence in which the amino acid sequence consisting of the (A-1) to (A-5) and the sequence of (A-6) encode one fusion protein through the following 2A peptide sequence of:
at least one of

T2A:
(SEQ ID NO: 211)
(GSG)EGRGSLLTCGDVEENPGP,
P2A:
(SEQ ID NO: 212)
(GSG)ATNFSLLKQAGDVEENPGP,
E2A
(SEQ ID NO: 213)
(GSG)QCTNYALLKLAGDVESNPGP,
and
F2A
(SEQ ID NO: 214)
(GSG)VKQTLNFDLLKLAGDVESNPGP.

It is preferable that the 2A peptide sequence causes ribosome skipping, and in a case where the sequence encoding a fusion protein is actually translated, a fusion protein which comprises an amino acid sequence consisting of independent (A-1) to (A-5) and a protein containing an independent the sequence of (A-6) are translated, and the two translated proteins form an MHC class I molecule or an MHC class II molecule.

In an embodiment of the present invention, the (a) above may include the following:
a sequence which is a polynucleotide encoding
(A) a fusion protein capable of presenting an antigen peptide outside membrane, in which the fusion protein comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of an MHC class Iα chain, β₂ microglobulin, an MHC class IIα chain, or an MHC class IIβ chain,
(A-4) a spacer sequence which may be optionally present,
(A-5) an amino acid sequence of a Tetraspanin or a membrane-binding domain thereof,
(A-5.5) a 2A peptide sequence, and
(A-6) an amino acid sequence of β₂ microglobulin, an MHC class Iα chain, an MHC class IIβ chain, or an MHC class IIα chain, in this order.

In an embodiment of the present invention, (a) may include the following:
a sequence which is a polynucleotide encoding
(A) a fusion protein capable of presenting an antigen peptide outside membrane, in which the fusion protein comprises an amino acid sequence containing, from an N-terminal side thereof,
(A-1) an MHC molecule-restricted antigen peptide,
(A-2) a spacer sequence which may be optionally present,
(A-3) an amino acid sequence of an MHC class IIβ chain,
(A-4) a spacer sequence which may be optionally present,
(A-5) an amino acid sequence of a Tetraspanin or a transmembrane domain thereof,
(A-5.5) a 2A peptide sequence, and
(A-6) an amino acid sequence of an MHC class IIα chain, in this order. As such an aspect, HLADR-1sc-TPI1-hCD81 (amino acid sequence: SEQ ID NO: 165; polynucleotide sequence: SEQ ID NO: 166) of Examples is exemplified.

In an embodiment of the present invention, the (a) above may contain a sequence encoding
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of β₂ microglobulin,
(A-4) a spacer sequence which may be optionally present, and
(A-5) an amino acid sequence of a Tetraspanin or a transmembrane domain thereof, in this order, and
a protein which comprises
(A-6) an amino acid sequence of an MHC class Iα chain.

In addition, in an embodiment of the present invention, the (a) above may contain a sequence encoding
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of an MHC class Iα chain,
(A-4) a spacer sequence which may be optionally present, and
(A-5) an amino acid sequence of a Tetraspanin or a transmembrane domain thereof, in this order, and
a protein which comprises
(A-6) an amino acid sequence of β₂ microglobulin.

In addition, in an embodiment of the present invention, the (a) above may contain a sequence encoding
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of an MHC class IIβ chain,
(A-4) a spacer sequence which may be optionally present, and
(A-5) an amino acid sequence of a Tetraspanin or a transmembrane domain thereof, in this order, and
a protein which comprises
(A-6) an amino acid sequence of an MHC class IIα chain.

In addition, in an embodiment of the present invention, the (a) above may contain a sequence encoding
(A) a protein complex capable of presenting an antigen peptide outside membrane, in which the protein complex contains:
a fusion protein which comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of an MHC class IIα chain,
(A-4) a spacer sequence which may be optionally present, and
(A-5) an amino acid sequence of a Tetraspanin or a transmembrane domain thereof, in this order, and
a protein which comprises
(A-6) an amino acid sequence of an MHC class IIβ chain.

In an embodiment of the present invention, (a) may include the following:
a sequence encoding
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence of SEQ ID NO: 5,
(A-3) an amino acid sequence of a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order, or
(A) a fusion protein capable of presenting an antigen peptide outside membrane and constituting a protein complex, in which the fusion protein comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order.

In an embodiment of the present invention, (a) may include the following:
a sequence encoding
(A) a fusion protein capable of presenting an antigen peptide outside membrane, in which the fusion protein comprises an amino acid sequence containing, from an N-terminal side thereof,
(A-1) an amino acid sequence of an MHC class I molecule-restricted antigen peptide,
(A-2) a spacer sequence of SEQ ID NO: 5,
(A-3) an amino acid sequence of a single chain MHC class I molecule of SEQ ID NO: 65 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order, or
(A) a fusion protein capable of presenting an antigen peptide outside membrane and constituting a protein complex, in which the fusion protein comprises an amino acid sequence containing, from an N-terminal side thereof,
(A-1) an amino acid sequence of an MHC class II molecule-restricted antigen peptide,
(A-2) a spacer sequence of SEQ ID NO: 39,
(A-3) an amino acid sequence of an MHC class IIβ chain of SEQ ID NO: 37 (or a sequence having an amino acid sequence identity thereto of 80% or more, preferably 90% or more, more preferably 95% or more, still more preferably 98% or more, and further still more preferably 99% or more), and
(A-5) an amino acid sequence of a Tetraspanin or a transmembrane domain thereof of SEQ ID NO: 15 (or a sequence having an amino acid sequence identity thereto of 80% or more, preferably 90% or more, more preferably 95% or more, still more preferably 98% or more, and further still more preferably 99% or more), in this order.

In an embodiment of the present invention, (a) may include the following:
(A) a sequence which is a polynucleotide encoding a fusion protein capable of presenting an antigen peptide outside membrane, in which the sequence contains, from a 5′ end,
(A-1) a sequence encoding an MHC class I molecule-restricted antigen peptide,
(A-2) a spacer sequence of SEQ ID NO: 6,
(A-3) a sequence encoding a single chain MHC class I molecule of SEQ ID NO: 66 (or a sequence having a sequence identity thereto of 80% or more, preferably 90% or more, more preferably 95% or more, still more preferably 98% or more, and further still more preferably 99% or more), and
(A-5) a sequence encoding a Tetraspanin or a transmembrane domain thereof of SEQ ID NO: 16 (or a sequence having a sequence identity thereto of 80% or more, preferably 90% or more, more preferably 95% or more, still more preferably 98% or more, and further still more preferably 99% or more), in this order, or
(A) a sequence which is a polynucleotide encoding a fusion protein capable of presenting an antigen peptide outside membrane and constituting a protein complex, in which the sequence contains, from a 5′ end,
(A-1) a polynucleotide encoding an MHC class II molecule-restricted antigen peptide,
(A-2) a spacer sequence of SEQ ID NO: 40,
(A-3) a sequence encoding an MHC class IIβ chain of SEQ ID NO: 38 (or a sequence having a sequence identity thereto of 80% or more, preferably 90% or more, more preferably 95% or more, still more preferably 98% or more, and further still more preferably 99% or more), and
(A-5) a sequence encoding a Tetraspanin or a transmembrane domain thereof of SEQ ID NO: 16 (or a sequence having a sequence identity thereto of 80% or more, preferably 90% or more, more preferably 95% or more, still more preferably 98% or more, and further still more preferably 99% or more), in this order.

In an embodiment of the present invention, the (a) above may include the following:
a sequence containing
(A) a sequence which is a polynucleotide encoding a fusion protein capable of presenting an antigen peptide outside membrane, in which the sequence contains, from a 5′ end,
(A-1) a polynucleotide encoding an MHC class I molecule-restricted antigen peptide,
(A-2) a spacer sequence of SEQ ID NO: 6,
(A-3) a sequence encoding a single chain MHC class I molecule of SEQ ID NO: 66 (or a sequence having a sequence identity thereto of 80% or more, preferably 90% or more, more preferably 95% or more, still more preferably 98% or more, and further still more preferably 99% or more), and
(A-5) a sequence encoding a Tetraspanin or a transmembrane domain thereof of SEQ ID NO: 16 (or a sequence having a sequence identity thereto of 80% or more, preferably 90% or more, more preferably 95% or more, still more preferably 98% or more, and further still more preferably 99% or more), in this order, or
(A) a sequence which is a polynucleotide encoding a fusion protein capable of presenting an antigen peptide outside membrane and constituting a protein complex, in which the sequence contains,
(A-1) a polynucleotide encoding an MHC class II molecule-restricted antigen peptide,
(A-2) a spacer sequence of SEQ ID NO: 40,
(A-3) a sequence encoding an MHC class IIβ chain of SEQ ID NO: 38 (or a sequence having a sequence identity thereto of 80% or more, preferably 90% or more, more preferably 95% or more, still more preferably 98% or more, and further still more preferably 99% or more), and
(A-5) a sequence encoding a Tetraspanin or a transmembrane domain thereof of SEQ ID NO: 16 (or a sequence having a sequence identity thereto of 80% or more, preferably 90% or more, more preferably 95% or more, still more preferably 98% or more, and further still more preferably 99% or more), in this order.

In an embodiment of the present invention, the (b) above may contain (B) a sequence encoding a fusion protein which comprises a first T-cell stimulatory cytokine and CD8 or a transmembrane domain thereof, the fusion protein being capable of presenting the first T-cell stimulatory cytokine outside membrane.

In an embodiment of the present invention, the (b) above may contain
(B) a sequence encoding a fusion protein which comprises an amino acid sequence containing, from an N-terminal side thereof,
(B-3) an amino acid sequence of a first T-cell stimulatory cytokine,
(B-4) a spacer sequence which may be optionally present, and
(B-5) an amino acid sequence of CD8 or a transmembrane domain thereof, in this order,
the fusion protein being capable of presenting the first T-cell stimulatory cytokine outside the membrane.

In an embodiment of the present invention, the (b) above may contain
(B) a sequence encoding a fusion protein comprising a first T-cell stimulatory cytokine and a partial sequence of a Tetraspanin and being capable of presenting the first T-cell stimulatory cytokine outside membrane, in which the partial sequence of the Tetraspanin contains at least two transmembrane domains and the first T-cell stimulatory cytokine is disposed between the two transmembrane domains, or
(B) a sequence encoding a fusion protein comprising a first T-cell stimulatory cytokine and MFG-E8 or a domain thereof and being capable of presenting the first T-cell stimulatory cytokine outside membrane.

In an embodiment of the present invention, the (b) above may contain
(B) a sequence encoding a fusion protein which comprises an amino acid sequence containing, from an N-terminal side thereof,
(B-1) a partial sequence of a Tetraspanin containing, from an N-terminal side thereof, a transmembrane domain 1, a small extracellular loop, a transmembrane domain 2, a small intracellular loop, and a transmembrane domain 3,
(B-2) a spacer sequence which may be optionally present,
(B-3) an amino acid sequence of a first T-cell stimulatory cytokine,
(B-4) a spacer sequence which may be optionally present, and
(B-5) a partial sequence of a Tetraspanin containing a transmembrane domain 4, in this order,
the fusion protein being capable of presenting the first T-cell stimulatory cytokine outside membrane, or
(B) a sequence encoding a fusion protein which comprises an amino acid sequence containing, from an N-terminal side thereof,
(B-3) an amino acid sequence of a first T-cell stimulatory cytokine,
(B-4) a spacer sequence which may be optionally present, and
(B-5) an amino acid sequence of MFG-E8 or a membrane-binding domain thereof, in this order,
the fusion protein being capable of presenting the first T-cell stimulatory cytokine outside membrane.

In an embodiment of the present invention, the (b) above may contain:
(B) a sequence encoding a fusion protein which comprises an amino acid sequence containing, from an N-terminal side thereof,
(B-1) a partial sequence of a Tetraspanin of SEQ ID NO: 57 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) an amino acid sequence of a first T-cell stimulatory cytokine which is IL-2 of SEQ ID NO: 25 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29,
(B-5) a partial sequence of a Tetraspanin of SEQ ID NO: 59 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the first T-cell stimulatory cytokine outside membrane:
(B) a sequence encoding a fusion protein which comprises an amino acid sequence containing, from an N-terminal side thereof,
(B-1) a partial sequence of a Tetraspanin of SEQ ID NO: 61 (or a sequence having an amino acid sequence identity thereto of 80% or more, preferably 90% or more, more preferably 95% or more, still more preferably 98% or more, and further still more preferably 99% or more),
(B-2) a spacer sequence of SEQ ID NO: 29,
(B-3) an amino acid sequence of a first T-cell stimulatory cytokine which is IL-4 of SEQ ID NO: 53 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) a partial sequence of a Tetraspanin of SEQ ID NO: 63 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the first T-cell stimulatory cytokine outside membrane; or
(B) a sequence encoding a fusion protein which comprises an amino acid sequence containing, from an N-terminal side thereof,
(B-3) an amino acid sequence of a second T-cell stimulatory cytokine which is TGF-β of SEQ ID NO: 73 (or a sequence having an amino acid sequence identity thereto of 80% or more, preferably 90% or more, more preferably 95% or more, still more preferably 98% or more, and further still more preferably 99% or more),
(B-4) a spacer sequence of SEQ ID NO: 29, and
(B-5) an amino acid sequence of MFG-E8 or a membrane-binding domain thereof of SEQ ID NO: 49 (or a sequence having an amino acid sequence identity thereto of 80% or more, preferably 90% or more, more preferably 95% or more, still more preferably 98% or more, and further still more preferably 99% or more), in this order,
the fusion protein being capable of presenting the second (or first) T-cell stimulatory cytokine outside membrane.

In an embodiment of the present invention, the (b) above may contain:
(B) a sequence which is a polynucleotide encoding a fusion protein capable of presenting a first T-cell stimulatory cytokine outside membrane, in which the sequence containing, from a 5′ end,
(B-1) a partial sequence of a Tetraspanin of SEQ ID NO: 58 (or a sequence having a sequence identity thereto of 80% or more, preferably 90% or more, more preferably 95% or more, still more preferably 98% or more, and further still more preferably 99% or more),
(B-2) a spacer sequence of SEQ ID NO: 30,
(B-3) a sequence encoding a first T-cell stimulatory cytokine which is IL-2 of SEQ ID NO: 26 (or a sequence having a sequence identity thereto of 80% or more, preferably 90% or more, more preferably 95% or more, still more preferably 98% or more, and further still more preferably 99% or more),
(B-4) a spacer sequence of SEQ ID NO: 30, and
(B-5) a partial sequence of a Tetraspanin of SEQ ID NO: 60 (or a sequence having a sequence identity thereto of 80% or more, preferably 90% or more, more preferably 95% or more, still more preferably 98% or more, and further still more preferably 99% or more), in this order;
(B) a sequence which is a polynucleotide encoding a fusion protein capable of presenting a first T-cell stimulatory cytokine outside membrane, in which the sequence containing, from a 5′ end,
(B-1) a partial sequence of a Tetraspanin of SEQ ID NO: 62 (or a sequence having a sequence identity thereto of 80% or more, preferably 90% or more, more preferably 95% or more, still more preferably 98% or more, and further still more preferably 99% or more),
(B-2) a spacer sequence of SEQ ID NO: 30,
(B-3) a first T-cell stimulatory cytokine which is IL-4 of SEQ ID NO: 54 (or a sequence having a sequence identity thereto of 80% or more, preferably 90% or more, more preferably 95% or more, still more preferably 98% or more, and further still more preferably 99% or more),
(B-4) a spacer sequence of SEQ ID NO: 30, and
(B-5) a partial sequence of a Tetraspanin of SEQ ID NO: 64 (or a sequence having a sequence identity thereto of 80% or more, preferably 90% or more, more preferably 95% or more, still more preferably 98% or more, and further still more preferably 99% or more), in this order;
(B) a sequence which is a polynucleotide encoding a fusion protein capable of presenting a second (or first) T-cell stimulatory cytokine outside membrane, in which the sequence containing, from a 5′ end,
(B-3) a second (or first) T-cell stimulatory cytokine which is TGF-β of SEQ ID NO: 74 (or a sequence having a sequence identity thereto of 80% or more, preferably 90% or more, more preferably 95% or more, still more preferably 98% or more, and further still more preferably 99% or more),
(B-4) a spacer sequence of SEQ ID NO: 30, and
(B-5) a sequence encoding MFG-E8 or a membrane-binding domain thereof of SEQ ID NO: 50 (or a sequence having a sequence identity thereto of 80% or more, preferably 90% or more, more preferably 95% or more, still more preferably 98% or more, and further still more preferably 99% or more), in this order;
(B) a sequence encoding a fusion protein capable of presenting the first (or second) T-cell stimulatory cytokine of SEQ ID NO: 31, 75, or 55 (or a sequence having an amino acid sequence identity thereto of 80% or more, preferably 90% or more, more preferably 95% or more, still more preferably 98% or more, and further still more preferably 99% or more) outside the membrane; or
(B) a sequence encoding a fusion protein capable of presenting the first (or second) T-cell stimulatory cytokine of SEQ ID NO: 32, 76, or 56 (or a sequence having a sequence identity thereto of 80% or more, preferably 90% or more, more preferably 95% or more, still more preferably 98% or more, and further still more preferably 99% or more) outside the membrane.

In a case where the T-cell stimulatory cytokine functions by a combination of hetero subunits, it is preferable that in the (b) above, the sequence of one subunit is used as the sequence of the T-cell stimulatory cytokine in the (B) above, and the sequence of the remaining subunit is separately contained in the (b), and in a case where the sequence is translated, it is preferable that the fusion protein of the (B) and the remaining subunit form an active T-cell stimulatory cytokine outside the membrane.

In an embodiment of the present invention, in a case where the T-cell stimulatory cytokine functions by a combination of hetero subunits, (b) may contain a sequence which is translated as a protein in which the fusion protein of (B) and the remaining subunits are fused.

The fusion protein of (B) and the remaining subunits may be fused through a spacer sequence or may be fused through a 2A peptide sequence.

In an embodiment of the present invention, (b) may contain
(B) a sequence encoding a fusion protein which comprises an amino acid sequence containing, from an N-terminal side thereof,
(B-6) an amino acid sequence of an IL-12β subunit,
(B-7) a spacer sequence which may be optionally present,
(B-3) an amino acid sequence of an IL-12a subunit,
(B-4) a spacer sequence which may be optionally present,
(B-5) an amino acid sequence of CD8 or a transmembrane domain thereof, in this order,
the fusion protein being capable of presenting IL-12 outside a membrane of a cell.

In an embodiment of the present invention, (b) may contain
(B) a sequence encoding a fusion protein which comprises an amino acid sequence containing, from an N-terminal side thereof,
(B-6) an amino acid sequence of an IL-12β subunit,
(B-7) a spacer sequence which may be optionally present,
(B-3) an amino acid sequence of an IL-12a subunit,
(B-4) a spacer sequence which may be optionally present,
(B-5) an amino acid sequence of MFG-E8 or a membrane-binding domain thereof, in this order,
the fusion protein being capable of presenting IL-12 outside a membrane of an extracellular vesicle.

As such an aspect, hIL-12sc-MFGe8 (amino acid sequence: SEQ ID NO: 177; polynucleotide sequence 178) is exemplified.

In an embodiment of the present invention, (c) may contain
(c) a sequence encoding a fusion protein comprising:
a T-cell costimulatory molecule containing a transmembrane domain; or
a T-cell costimulatory molecule, a Tetraspanin or a transmembrane domain thereof or MFG-E8 or a domain thereof.

In an embodiment of the present invention, (c) may contain
(C) a sequence encoding a fusion protein which comprises an amino acid sequence containing, from an N-terminal side thereof,
(C-1) an amino acid sequence of a T-cell costimulatory molecule,
(C-2) a spacer sequence which may be optionally present, and
(C-3) an amino acid sequence of a Tetraspanin or a membrane-binding domain thereof,
the fusion protein being capable of allowing the T-cell costimulatory molecule to interact with T cells.

In an embodiment of the present invention, (c) may contain
(C) a sequence encoding a fusion protein which comprises an amino acid sequence containing, from an N-terminal side thereof,
(C-1) an amino acid sequence of a T-cell costimulatory molecule which is CD80 of SEQ ID NO: 67 (or a sequence having an amino acid sequence identity thereto of 80% or more, preferably 90% or more, more preferably 95% or more, still more preferably 98% or more, and further still more preferably 99% or more), and
(C-3) an amino acid sequence of a Tetraspanin or a transmembrane domain thereof of SEQ ID NO: 21 (or a sequence having an amino acid sequence identity thereto of 80% or more, preferably 90% or more, more preferably 95% or more, still more preferably 98% or more, and further still more preferably 99% or more), in this order,
the fusion protein being capable of allowing the T-cell costimulatory molecule to interact with T cells.

In an embodiment of the present invention, (c) may contain
(C) a sequence encoding a fusion protein capable of allowing the T-cell costimulatory molecule of SEQ ID NO: 23 (or a sequence having an amino acid sequence identity thereto of 80% or more, preferably 90% or more, more preferably 95% or more, still more preferably 98% or more, and further still more preferably 99% or more, to interact with T cells.

In an embodiment of the present invention, (c) may contain
(C) a sequence containing, from a 5′ end,
(C-1) a sequence encoding a T-cell costimulatory molecule which is CD80 of SEQ ID NO: 68 (or a sequence having a sequence identity thereto of 80% or more, preferably 90% or more, more preferably 95% or more, still more preferably 98% or more, and further still more preferably 99% or more), and
(C-3) a sequence encoding a Tetraspanin or a membrane-binding domain thereof of SEQ ID NO: 22 (or a sequence having a sequence identity thereto of 80% or more, preferably 90% or more, more preferably 95% or more, still more preferably 98% or more, and further still more preferably 99% or more), in this order.

In an embodiment of the present invention, (c) may contain
(C) a sequence encoding a fusion protein capable of allowing the T-cell costimulatory molecule of SEQ ID NO: 24 (or a sequence having a sequence identity thereto of 80% or more, preferably 90% or more, more preferably 95% or more, still more preferably 98% or more, and further still more preferably 99% or more, to interact with T cells.

As an embodiment of the present invention, (d) may contain
(D) a sequence encoding a fusion protein which comprises the antigen-presenting MHC molecule, the at least one T-cell stimulatory cytokine or subunit thereof, and a membrane protein capable of being localized to membrane of a cell or an extracellular vesicle or a transmembrane domain thereof or a protein capable of binding to membrane of a cell or an extracellular vesicle or a membrane-binding domain thereof.

The membrane protein capable of being localized to membrane of an extracellular vesicle or the protein capable of binding to membrane of an extracellular vesicle may be a Tetraspanin or MFG-E8. The membrane protein capable of being localized to membrane of a cell or the protein capable of binding to membrane of a cell may be CD8, and an MHC molecule containing a transmembrane domain may perform this function.

As an embodiment of the present invention, (d) may contain
(D) a sequence encoding a fusion protein which comprises an amino acid sequence containing, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule,
(D-4) a spacer sequence which may be optionally present, and
(D-5) an amino acid sequence of a fusion peptide containing CD8 or a transmembrane domain thereof and the at least one T-cell stimulatory cytokine, in this order.

As an embodiment of the present invention, (d) may contain
(D) a sequence encoding a fusion protein which comprises an amino acid sequence containing, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule containing a transmembrane domain,
(D-4) a spacer sequence which may be optionally present, and
(D-5) an amino acid sequence of a fusion peptide containing at least one T-cell stimulatory cytokine, in this order.

As an embodiment of the present invention, (d) may contain
(D)
a sequence encoding a fusion protein which comprises an amino acid sequence containing, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule,
(D-4) a spacer sequence which may be optionally present, and
(D-5) an amino acid sequence of a fusion peptide comprising a Tetraspanin or a transmembrane domain thereof or MFG-E8 or a transmembrane domain thereof, and the at least one T-cell stimulatory cytokine or subunit thereof, in this order (here, the respective constituent elements (D-1) to (D-5) have the aspect described in the present specification).

Alternatively, as an embodiment of the present invention, (d) may contain
(D) a sequence encoding a fusion protein which comprises an amino acid sequence containing, from an N-terminal side thereof,
(D-1) an amino acid sequence of a fusion peptide comprising a Tetraspanin or a transmembrane domain thereof or MFG-E8 or a transmembrane domain thereof, and the at least one T-cell stimulatory cytokine or subunit thereof,
(D-2) a spacer sequence which may be optionally present,
(D-3) an amino acid sequence of a single chain MHC molecule,
(D-4) a spacer sequence which may be optionally present, and
(D-5) an amino acid sequence of an MHC molecule-restricted antigen peptide, in this order (here, the respective constituent elements (D-1) to (D-5) have the aspect described in the present specification).

In the aspect, the fusion peptide comprising a Tetraspanin or a transmembrane domain thereof or MFG-E8 or a transmembrane domain thereof, and the at least one T-cell stimulatory cytokine or subunit thereof may comprise an amino acid sequence encoding, from an N-terminal side thereof,
(1) a partial sequence of a Tetraspanin containing a transmembrane domain 1, a small extracellular loop, a transmembrane domain 2, a small intracellular loop, and a transmembrane domain 3,
(2) a spacer sequence which may be optionally present,
(3) the at least one T-cell stimulatory cytokine or subunit thereof,
(4) a spacer sequence which may be optionally present, and
(5) a partial sequence of a Tetraspanin containing a transmembrane domain 4, in this order, or
the fusion peptide comprising a Tetraspanin or a transmembrane domain thereof or MFG-E8 or a transmembrane domain thereof, and the at least one T-cell stimulatory cytokine or subunit thereof may comprise an amino acid sequence encoding, from an N-terminal side thereof,
(1) the at least one T-cell stimulatory cytokine or subunit thereof,
(2) a spacer sequence which may be optionally present, and
(3) MFG-E8, or a membrane-binding domain thereof, in this order.

Here, the MHC molecule-restricted antigen peptide may be an MHC class I molecule-restricted antigen peptide, the single chain MHC molecule may contain an extracellular domain of an MHC class Iα chain, the MHC molecule-restricted antigen peptide may be an MHC class II molecule-restricted antigen peptide, and the single chain MHC molecule may contain an extracellular domain of an MHC class IIα chain and/or an extracellular domain of an MHC class IIβ chain.

In an embodiment of the present invention, there is provided a polynucleotide containing the sequence defined as (a) and the sequence defined as (b).

The polynucleotide may further contain the sequence defined as (c).

In an embodiment of the present invention,
there is provided a polynucleotide containing the sequence defined as (d).

Examples of such a sequence include a nucleic acid sequence of SEQ ID NO: 136 encoding an amino acid sequence of SEQ ID NO: 135.

In the aspect, the polynucleotide may contain the sequence defined as (c).

In an embodiment of the present invention, there is provided a polynucleotide containing the sequence defined as (e).

In an embodiment of the present invention, as for the (A), (B), and (C) above, (A) and (B) may be fused to form a polynucleotide encoding fusion proteins to be one molecule, (B) and (C) may be fused to form a polynucleotide encoding fusion proteins to be one molecule, and (A), (B), and (C) may be fused to form a polynucleotide encoding fusion proteins to be one molecule. Such a polynucleotide may encode one fusion protein with or without a spacer sequence between (A), (B), and (C). The sequence encoding fusion proteins (A) to (C) may contain a sequence fused through at least one sequence independently selected from the following 2A peptide sequences:

T2A:
(SEQ ID NO: 211)
(GSG)EGRGSLLTCGDVEENPGP,
P2A:
(SEQ ID NO: 212)
(GSG)ATNFSLLKQAGDVEENPGP,
E2A
(SEQ ID NO: 213)
(GSG)QCTNYALLKLAGDVESNPGP,
and
F2A
(SEQ ID NO: 214)
(GSG)VKQTLNFDLLKLAGDVESNPGP.

The 2A peptide sequence causes ribosome skipping, and in a case where the sequence encoding a fusion protein is actually translated, it may be present in a cell or an extracellular vesicle as independent (A), (B), and (C) molecules.

Alternatively, the polynucleotide in an embodiment of the present invention may encode a fusion protein obtained by functionally fusing the (A), (B), and (C) above by sharing an element for localizing the proteins thereof in the cell or the extracellular vesicle, that is, a site of a “membrane protein capable of being expressed in membrane of a cell or an extracellular vesicle or a transmembrane domain thereof” or a “protein capable of binding to membrane of a cell or an extracellular vesicle or a domain thereof”.

For example, in an embodiment of the present invention,
the polynucleotide may be a polynucleotide encoding (D) a fusion protein comprising:
(1) an antigen-presenting MHC molecule;
(2) at least one T-cell stimulatory cytokine; and
(3) a “membrane protein capable of being expressed in membrane of a cell or an extracellular vesicle or a transmembrane domain thereof” or a “protein capable of binding to membrane of a cell or an extracellular vesicle or a domain thereof”,
the fusion protein being fused in a form of sharing the site of the “membrane protein capable of being expressed in membrane of a cell or an extracellular vesicle or the transmembrane domain thereof” or the “protein capable of binding to membrane of a cell or an extracellular vesicle or the domain thereof” in (A) and (B);
a polynucleotide encoding a fusion protein (F) containing:
(1) an antigen-presenting MHC molecule;
(2) a T-cell costimulatory molecule; and
(3) a “membrane protein capable of being expressed in membrane of a cell or an extracellular vesicle or a transmembrane domain thereof” or a “protein capable of binding to membrane of a cell or an extracellular vesicle or a domain thereof”,
the fusion protein being fused in a form of sharing the site of the “membrane protein capable of being expressed in membrane of a cell or an extracellular vesicle or the transmembrane domain thereof” or the “protein capable of binding to membrane of a cell or an extracellular vesicle or the domain thereof” in (A) and (C);
a polynucleotide encoding a fusion protein (G) containing:
(1) at least one T-cell stimulatory cytokine;
(2) a T-cell costimulatory molecule; and
(3) a “membrane protein capable of being expressed in membrane of a cell or an extracellular vesicle or a transmembrane domain thereof” or a “protein capable of binding to membrane of a cell or an extracellular vesicle or a domain thereof”,
the fusion protein being fused in a form of sharing the site of the “membrane protein capable of being expressed in membrane of a cell or an extracellular vesicle or the transmembrane domain thereof” or the “protein capable of binding to membrane of a cell or an extracellular vesicle or the domain thereof” in (B) and (C); or,
a polynucleotide encoding a fusion protein (E) containing:
(1) an antigen-presenting MHC molecule;
(2) at least one T-cell stimulatory cytokine;
(2) a T-cell costimulatory molecule; and
(4) a “membrane protein capable of being expressed in membrane of a cell or an extracellular vesicle or a transmembrane domain thereof” or a “protein capable of binding to membrane of a cell or an extracellular vesicle or a domain thereof”,
the fusion protein being fused in a form of sharing the site of the “membrane protein capable of being expressed in membrane of a cell or an extracellular vesicle or the transmembrane domain thereof” or the “protein capable of binding to membrane of a cell or an extracellular vesicle or the domain thereof” in (A) to (C).

In an embodiment of the present invention, the polynucleotide may be a polynucleotide encoding a fusion protein which comprises an antigen-presenting MHC molecule and at least one T-cell stimulatory cytokine and is capable of presenting the antigen and the T-cell stimulatory cytokine, the fusion protein being the fusion protein (D) having the functions of the constitutional requirement (A) and the constitutional requirement (B) using the protein of the constitutional requirement (B) which contains a first T-cell stimulatory cytokine and is capable of presenting the first T-cell stimulatory cytokine outside membrane, instead of the membrane protein capable of being expressed in membrane of a cell or an extracellular vesicle or the transmembrane domain thereof or the protein capable of binding to membrane of a cell or an extracellular vesicle of the constitutional requirement (A).

Such a fusion protein (D) having the functions of the constitutional requirement (A) and the constitutional requirement (B) may be a fusion protein which comprises an antigen-presenting MHC molecule and at least one T-cell stimulatory cytokine and is capable of presenting the antigen and the T-cell stimulatory cytokine outside membrane.

The fusion protein may comprise the antigen-presenting MHC molecule, the at least one T-cell stimulatory cytokine, and a membrane protein capable of being localized to membrane of a cell or an extracellular vesicle or a transmembrane domain thereof or a protein capable of binding to membrane of a cell or an extracellular vesicle or a membrane-binding domain thereof.

In the fusion protein (D) having the functions of the constitutional requirement (A) and the constitutional requirement (B), the membrane protein capable of being localized to membrane of an extracellular vesicle or the protein capable of binding to membrane of an extracellular vesicle may be a Tetraspanin or MFG-E8.

In the fusion protein (D) having the functions of the constitutional requirement (A) and the constitutional requirement (B), the membrane protein capable of being localized to membrane of an extracellular vesicle or the protein capable of binding to membrane of an extracellular vesicle may be a Tetraspanin or MFG-E8.

The fusion protein may also comprise an amino acid sequence encoding, from an N-terminal side thereof,
(D-1) an amino acid sequence of an MHC molecule-restricted antigen peptide,
(D-2) a spacer sequence which may be optionally present,
(D-3) a single chain MHC molecule,
(D-4) a spacer sequence which may be optionally present, and
(D-5) a fusion peptide comprising a Tetraspanin or a transmembrane domain thereof or MFG-E8 or a transmembrane domain thereof, and the at least one T-cell stimulatory cytokine, in this order.

The fusion protein may also comprise an amino acid sequence encoding, from an N-terminal side thereof,
(D-1) a fusion peptide comprising a Tetraspanin or a transmembrane domain thereof or MFG-E8 or a transmembrane domain thereof, and the at least one T-cell stimulatory cytokine,
(D-2) a spacer sequence which may be optionally present,
(D-3) a single chain MHC molecule,
(D-4) a spacer sequence which may be optionally present, and
(D-5) an MHC molecule-restricted antigen peptide, in this order.

Here, the fusion peptide may also comprise an amino acid sequence encoding, from an N-terminal side thereof,
(1) a partial sequence of a Tetraspanin containing a transmembrane domain 1, a small intracellular loop, a transmembrane domain 2, a small extracellular loop, and a transmembrane domain 3,
(2) a spacer sequence which may be optionally present,
(3) an amino acid sequence of the at least one T-cell stimulatory cytokine,
(4) a spacer sequence which may be optionally present, and
(5) a partial sequence of a Tetraspanin containing a transmembrane domain 4, in this order.

The fusion peptide may also comprise an amino acid sequence encoding, from an N-terminal side thereof,
(1) the at least one T-cell stimulatory cytokine,
(2) a spacer sequence which may be optionally present, and
(3) MFG-E8, in this order.

In an embodiment of the present invention, the MHC molecule-restricted antigen peptide is an MHC class I molecule-restricted antigen peptide, the single chain MHC molecule may contain an extracellular domain of an MHC class Iα chain, the MHC molecule-restricted antigen peptide is an MHC class II molecule-restricted antigen peptide, and the single chain MHC molecule may contain an extracellular domain of an MHC class IIα chain and/or an extracellular domain of an MHC class IIβ chain.

In the aspect containing the fusion protein (D) having the functions of the constitutional requirement (A) and the constitutional requirement (B):
(C) a protein containing at least one T-cell costimulatory molecule and capable of allowing the T-cell costimulatory molecule to interact with T cells may also be contained in the membrane:
the protein capable of interacting with T cells may also contain the at least one T-cell costimulatory molecule, and a membrane protein capable of being expressed on a membrane of an extracellular vesicle or a transmembrane domain thereof or a protein capable of binding to a membrane of an extracellular vesicle or a domain thereof; and
the protein capable of interacting with T cells may also contain the at least one T-cell costimulatory molecule, and a Tetraspanin or a transmembrane domain thereof or MFG-E8 or a domain thereof.

### Vector and Kit

In an embodiment of the present invention, there is provided a vector comprising at least one polynucleotide selected from the polynucleotides described in the present specification.

The “vector” used in the present specification means any vector (examples thereof include, but are not limited to, a plasmid vector, a cosmid vectors a phage vector such as a phage, a viral vector such as an adenovirus vector or a baculovirus vector, and an artificial chromosome vector). The vector includes an expression vector, a cloning vector, and the like. The expression vector may generally contain a desired coding sequence and an appropriate polynucleotide required for expression of an operably linked coding sequence in a host organism (for example, a plant, an insect, an animal, or the like) or in an in vitro expression system. The cloning vector may be used to manipulate and/or amplify a desired polynucleotide fragment. The cloning vector may delete functional sequences required for expression of a desired polynucleotide fragment.

In an embodiment of the invention, all the polynucleotides described in the present specification may be inserted into the same vector, or two or more polynucleotides may be inserted into different vectors, as long as they can be operably inserted. In an embodiment of the present invention, there is provided a kit containing a combination of two or more vectors containing at least one polynucleotide selected from the polynucleotides described in the present specification.

### Transformed Cells

In an embodiment of the present invention, there is provided a cell transformed with a vector comprising,
(i) a polynucleotide encoding the fusion protein or the protein complex of (A) described in the present specification,
(ii) a polynucleotide encoding the fusion protein of (B) described in the present specification, and
(iii) a polynucleotide encoding the fusion protein of (C) described in the present specification.

In an embodiment of the present invention, there is provided a cell transformed with a single vector or a combination of two or more vectors, the vector comprising,
(i) a polynucleotide encoding the fusion protein or the protein complex of (A) described in the present specification,
(ii) a polynucleotide encoding the fusion protein of (B) described in the present specification, and optionally,
(iii) a polynucleotide encoding the fusion protein of (C) described in the present specification.

In the cell of an embodiment of the present invention, as for the (A), (B), and (C) above, the cell may be transformed with a vector comprising a polynucleotide encoding fusion proteins to be one molecule obtained by fusing (A) and (B), a vector comprising a polynucleotide encoding fusion proteins to be one molecule obtained by fusing (B) and (C), or a vector comprising a polynucleotide encoding fusion proteins to be one molecule obtained by fusing (A), (B), and (C). Such a polynucleotide may encode one fusion protein with or without a spacer sequence between (A), (B), and (C).

Alternatively, the polynucleotide may encode a fusion protein obtained by functionally fusing the (A), (B), and (C) above by sharing an element for localizing the proteins thereof in the extracellular vesicle, that is, a site of a “membrane protein capable of being expressed in membrane of an extracellular vesicle or a transmembrane domain thereof” or a “protein capable of binding to membrane of an extracellular vesicle or a domain thereof”.

For example, in an embodiment of the present invention,
the cell may be transformed with a vector comprising a polynucleotide encoding a fusion protein (D) comprising:
(1) an antigen-presenting MHC molecule;
(2) at least one T-cell stimulatory cytokine; and
(3) a “membrane protein capable of being expressed in membrane of an extracellular vesicle or a transmembrane domain thereof” or a “protein capable of binding to membrane of an extracellular vesicle or a domain thereof”,
the fusion protein being fused in a form of sharing the site of the “membrane protein capable of being expressed in membrane of an extracellular vesicle or the transmembrane domain thereof” or the “protein capable of binding to membrane of an extracellular vesicle or the domain thereof” in (A) and (B);
a vector comprising a polynucleotide encoding a fusion protein (F) comprising:
(1) an antigen-presenting MHC molecule;
(2) a T-cell costimulatory molecule; and
(3) a “membrane protein capable of being expressed in membrane of an extracellular vesicle or a transmembrane domain thereof” or a “protein capable of binding to membrane of an extracellular vesicle or a domain thereof”,
the fusion protein being fused in a form of sharing the site of the “membrane protein capable of being expressed in membrane of an extracellular vesicle or the transmembrane domain thereof” or the “protein capable of binding to membrane of an extracellular vesicle or the domain thereof” in (A) and (C);
a vector comprising a polynucleotide encoding a fusion protein (G) comprising:
(1) at least one T-cell stimulatory cytokine;
(2) a T-cell costimulatory molecule; and
(3) a “membrane protein capable of being expressed in membrane of an extracellular vesicle or a transmembrane domain thereof” or a “protein capable of binding to membrane of an extracellular vesicle or a domain thereof”,
the fusion protein being fused in a form of sharing the site of the “membrane protein capable of being expressed in membrane of an extracellular vesicle or the transmembrane domain thereof” or the “protein capable of binding to membrane of an extracellular vesicle or the domain thereof” in (B) and (C);
or,
a vector comprising a polynucleotide encoding a fusion protein (E) comprising:
(1) an antigen-presenting MHC molecule;
(2) at least one T-cell stimulatory cytokine;
(3) a T-cell costimulatory molecule; and
(4) a “membrane protein capable of being expressed in membrane of an extracellular vesicle or a transmembrane domain thereof” or a “protein capable of binding to membrane of an extracellular vesicle or a domain thereof”,
the fusion protein being fused in a form of sharing the site of the “membrane protein capable of being expressed in membrane of an extracellular vesicle or the transmembrane domain thereof” or the “protein capable of binding to membrane of an extracellular vesicle or the domain thereof” in (A) to (C).

Alternatively, in an embodiment of the present invention,
there is provided a cell transformed with a vector comprising,
(iv) a polynucleotide encoding the fusion protein of (D) described in the present specification, in which the fusion protein contains an antigen-presenting MHC molecule and at least one T-cell stimulatory cytokine and is capable of presenting the antigen and the T-cell stimulatory cytokine outside membrane, the fusion protein being the fusion protein having the functions of the constitutional requirement (A) and the constitutional requirement (B) using the protein of the constitutional requirement (B) which contains a first T-cell stimulatory cytokine and is capable of presenting the first T-cell stimulatory cytokine outside membrane, instead of the membrane protein capable of being expressed in membrane of an extracellular vesicle or the transmembrane domain thereof or the protein capable of binding to membrane of an extracellular vesicle of the constitutional requirement (A).

The expression “transformed with a single vector or a combination of two or more vectors” means that, for example, the cell may be transformed with a single vector in which all the polynucleotides (i) to (iv) are inserted into the same vector, or may be transformed with a combination of two or more vectors in which two or more of the polynucleotides (i) to (iv) are inserted into different vectors.

In a case where (A) is a fusion protein, examples of “a single vector or a combination of two or more vectors” include the followings:
a vector comprising a polynucleotide encoding the fusion protein of (A) and a polynucleotide encoding the fusion protein of (B);
a combination of a vector comprising a polynucleotide encoding the fusion protein of (A) and a vector comprising a polynucleotide encoding the fusion protein of (B);
a vector comprising a polynucleotide encoding the fusion protein of (A), a polynucleotide encoding the fusion protein of (B), and a polynucleotide encoding the fusion protein of (C);
a combination of a vector comprising a polynucleotide encoding the fusion protein of (A) and a polynucleotide encoding the fusion protein of (B), and a vector comprising a polynucleotide encoding the fusion protein of (C);
a combination of a vector comprising a polynucleotide encoding the fusion protein of (A) and a polynucleotide encoding the fusion protein of (C), and a vector comprising a polynucleotide encoding the fusion protein of (B);
a combination of a vector comprising a polynucleotide encoding the fusion protein of (B) and a polynucleotide encoding the fusion protein of (C), and a vector comprising a polynucleotide encoding the fusion protein of (A); and
a combination of a vector comprising a polynucleotide encoding the fusion protein of (A), a vector comprising a polynucleotide encoding the fusion protein of (B), and a vector comprising a polynucleotide encoding the fusion protein of (C).

Alternatively, in a case where (A) is a protein complex, examples of “a single vector or a combination of two or more vectors” include the followings:
a vector comprising a polynucleotide encoding a fusion protein comprising an amino acid sequence consisting of (A-1) to (A-5), a polynucleotide encoding a protein comprising (A-6), and a polynucleotide encoding the fusion protein of (B);
a combination of a vector comprising a polynucleotide encoding a fusion protein comprising an amino acid sequence consisting of (A-1) to (A-5) and a polynucleotide encoding a protein comprising (A-6), and a vector comprising a polynucleotide encoding the fusion protein of (B);
a combination of a vector comprising a polynucleotide encoding a fusion protein comprising an amino acid sequence consisting of (A-1) to (A-5) and a polynucleotide encoding the fusion protein of (B), and a vector comprising a polynucleotide encoding a protein comprising (A-6);
a combination of a vector comprising a polynucleotide encoding a fusion protein comprising an amino acid sequence consisting of (A-1) to (A-5) and a vector comprising a polynucleotide encoding a protein comprising (A-6) and a polynucleotide encoding the fusion protein of (B);
a combination of a vector comprising a polynucleotide encoding a fusion protein comprising an amino acid sequence consisting of (A-1) to (A-5), a vector comprising a polynucleotide encoding a protein comprising (A-6), and a vector comprising a polynucleotide encoding the fusion protein of (B);
a vector comprising a polynucleotide encoding a fusion protein comprising an amino acid sequence consisting of (A-1) to (A-5), a polynucleotide encoding a protein comprising (A-6), a polynucleotide encoding the fusion protein of (B), and a polynucleotide encoding the fusion protein of (C);
a combination of a vector comprising a polynucleotide encoding a fusion protein comprising an amino acid sequence consisting of (A-1) to (A-5), a polynucleotide encoding a protein comprising (A-6), and a polynucleotide encoding the fusion protein of (B), and a vector comprising a polynucleotide encoding the fusion protein of (C);
a combination of a vector comprising a polynucleotide encoding a fusion protein comprising an amino acid sequence consisting of (A-1) to (A-5), a polynucleotide encoding the fusion protein of (B), and a polynucleotide encoding the fusion protein of (C), and a vector comprising a polynucleotide encoding a protein comprising (A-6);
a combination of a vector comprising a polynucleotide encoding a protein comprising (A-6), a polynucleotide encoding the fusion protein of (B), and a polynucleotide encoding the fusion protein of (C), and a vector comprising a polynucleotide encoding a fusion protein comprising an amino acid sequence consisting of (A-1) to (A-5);
a combination of a vector comprising a polynucleotide encoding a fusion protein comprising an amino acid sequence consisting of (A-1) to (A-5), a polynucleotide encoding a protein comprising (A-6), and a polynucleotide encoding the fusion protein of (C), and a vector comprising a polynucleotide encoding the fusion protein of (B);
a combination of a vector comprising a polynucleotide encoding a fusion protein comprising an amino acid sequence consisting of (A-1) to (A-5) and a polynucleotide encoding a protein comprising (A-6), and a vector comprising a polynucleotide encoding the fusion protein of (B) and a polynucleotide encoding the fusion protein of (C);
a combination of a vector comprising a polynucleotide encoding a fusion protein comprising an amino acid sequence consisting of (A-1) to (A-5) and a polynucleotide encoding the fusion protein of (B), and a vector comprising a polynucleotide encoding a protein comprising (A-6) and a polynucleotide encoding the fusion protein of (C);
a combination of a vector comprising a polynucleotide encoding a fusion protein comprising an amino acid sequence consisting of (A-1) to (A-5) and a polynucleotide encoding the fusion protein of (C), and a vector comprising a polynucleotide encoding a protein comprising (A-6) and a polynucleotide encoding the fusion protein of (B);
a combination of a vector comprising a polynucleotide encoding a fusion protein comprising an amino acid sequence consisting of (A-1) to (A-5), a vector comprising a polynucleotide encoding a protein comprising (A-6), and a vector comprising a polynucleotide encoding the fusion protein of (B) and a polynucleotide encoding the fusion protein of (C);
a combination of a vector comprising a polynucleotide encoding a fusion protein comprising an amino acid sequence consisting of (A-1) to (A-5), a vector comprising a polynucleotide encoding the fusion protein of (B), and a vector comprising a polynucleotide encoding a protein comprising (A-6) and a polynucleotide encoding the fusion protein of (C);
a combination of a vector comprising a polynucleotide encoding a fusion protein comprising an amino acid sequence consisting of (A-1) to (A-5), a vector comprising a polynucleotide encoding the fusion protein of (C), and a vector comprising a polynucleotide encoding a protein comprising (A-6) and a polynucleotide encoding the fusion protein of (B);
a combination of a vector comprising a polynucleotide encoding a protein comprising (A-6), a vector comprising a polynucleotide encoding the fusion protein of (B), and a vector comprising a polynucleotide encoding a fusion protein comprising an amino acid sequence consisting of (A-1) to (A-5) and a polynucleotide encoding the fusion protein of (C);
a combination of a vector comprising a polynucleotide encoding a protein comprising (A-6), a vector comprising a polynucleotide encoding the fusion protein of (C), and a vector comprising a polynucleotide encoding a fusion protein comprising an amino acid sequence consisting of (A-1) to (A-5) and a polynucleotide encoding the fusion protein of (B);
a combination of a vector comprising a polynucleotide encoding the fusion protein of (B), a vector comprising a polynucleotide encoding the fusion protein of (C), and a vector comprising a polynucleotide encoding a fusion protein comprising an amino acid sequence consisting of (A-1) to (A-5) and a polynucleotide encoding a protein comprising (A-6); and
a combination of a vector comprising a polynucleotide encoding a fusion protein comprising an amino acid sequence consisting of (A-1) to (A-5), a vector comprising a polynucleotide encoding a protein comprising (A-6), a vector comprising a polynucleotide encoding the fusion protein of (B), and a vector comprising a polynucleotide encoding the fusion protein of (C).

The cell to be transformed is not particularly limited as long as the antigen-presenting extracellular vesicle described in the present specification can be obtained after the transformation, and may be a primary cultured cell or an established cell, which may be a normal cell or a lesion cell containing cancerous or tumorigenic cells. In addition, the origin of the cell to be transformed is not particularly limited, and examples thereof include cells derived from animals such as mammals, for example, rodents such as a mouse, a rat, a hamster, and a guinea pig: lagomorph such as a rabbit: ungulates such as a pig, a cow, a goat, a horse, and a sheep: carnivora such as a dog and a cat; and primates such as a human, a monkey, a rhesus monkey, a crab-eating macaque, a marmoset, an orangutan, and a chimpanzee, plant-derived cells, and insect-derived cells. The cell to be transformed is preferably an animal-derived cell.

Examples of the animal-derived cells include, but are not limited to, human embryonic kidney cells (including HEK293T cells and the like), human FL cells, Chinese hamster ovary cells (CHO cells), COS-7, Vero, mouse L cells, and rat GH3.

A method for transforming the cell is not particularly limited as long as it is a method capable of introducing a target polynucleotide into a cell. For example, the method for transforming the cell may be an electroporation method, a microinjection method, a calcium phosphate method, a cationic lipid method, a method using a liposome, a method using a non-liposomal material such as polyethyleneimine, a viral infection method, or the like.

The transformed cell may be a transformed cell transiently expressing the fusion protein or protein complex of (A), (B), (C), (D), (E), (F), and/or (G), or a transformed cell (stable cell strain) stably expressing the fusion protein or protein complex of (A), (B), (C), (D), (E), (F), and/or (G).

The culture conditions of the cell to be transformed are not particularly limited. For example, when the transformed cell is an animal-derived cell, for example, a medium generally used for cell culture or the like (for example, an RPMI1640 medium, an Eagle's MEM medium, a Dulbecco's modified Eagle medium (DMEM medium), a Ham F12 medium, or any combination thereof), a medium obtained by adding other components such as fetal bovine serum, antibiotics, and amino acids, or the like may be used, and the cell may be cultured (for example, under being left or shaking), for example, in the presence of about 1 to about 10% (preferably about 2 to about 5%) of CO₂ at about 30 to about 40° C. (preferably about 37° C.) for a predetermined time (for example, about 0.5 hours to about 240 hours (preferably about 5 to about 120 hours, and more preferably about 12 to about 72 hours)).

A culture supernatant obtained by culturing the transformed cell may comprise the antigen-presenting extracellular vesicles described in the present specification. Therefore, when the transformed cell is cultured to obtain the antigen-presenting extracellular vesicles described in the present specification, a medium (for example, a Dulbecco's modified Eagle medium or the like containing about 1 to about 5% fetal bovine serum from which exosomes are removed) from which extracellular vesicles such as exosomes are removed may be used, if necessary.

### Culture Supernatant

In an embodiment of the present invention, a culture supernatant obtained by culturing the transformed cell described in the present specification is provided.

The antigen-presenting extracellular vesicles contained in the culture supernatant described in the present specification can be further collected, for example, by purifying (for example, centrifugation, chromatography, and the like), concentrating, and isolating the culture supernatant.

In an embodiment of the present invention, antigen-presenting extracellular vesicles obtained from the culture supernatant described in the present specification are provided.

### Method for Preparing Antigen-Presenting Cells or Antigen-Presenting Extracellular Vesicles Described in Present Specification

The antigen-presenting cells or the antigen-presenting extracellular vesicles described in the present specification may be obtained by, for example, means such as genetic recombination techniques known to those skilled in the art (for example, by the method described below or by the method described in Examples), but the present invention is not limited thereto.

A polynucleotide encoding the proteins of (A) and (B) described above (or (D) instead of (A) and (B)), and if necessary. (C), respectively, is obtained (or (A) and (a) to (d)) by normal genetic recombination techniques, and can be operably inserted into the same or different vectors. In a case where two or more polynucleotides encoding the proteins of (A) and (B) (or (D) instead of (A) and (B)), and if necessary. (C), respectively, are inserted into the same vector, each of the polynucleotides may be operably linked to the same or different promoters.

The obtained single or two or more vectors for an antigen-presenting cell can be transformed into cells simultaneously or sequentially to obtain antigen-presenting cells (may be transformed cells that transiently express these fusion proteins, or may be transformed cells (stable strains) that stably express these fusion proteins).

The obtained single or two or more vectors for an antigen-presenting extracellular vesicle can be transformed into cells simultaneously or sequentially to obtain transformed cells (may be transformed cells that transiently express these fusion proteins, or may be transformed cells (stable strains) that stably express these fusion proteins). The obtained transformed cells are cultured under desired conditions to obtain a culture supernatant, and the obtained culture supernatant is purified (for example, purification using centrifugation, antibodies (for example, antibodies recognizing a protein or the like contained in membrane of an extracellular vesicle), chromatography, flow cytometry, or the like), concentrated (for example, ultrafiltration or the like), and dried, such that the antigen-presenting extracellular vesicles described in the present specification can be obtained.

Alternatively, in a case where soluble proteins are used as the proteins of (A) and (B) (or (D) instead of (A) and (B)) described above, and if necessary. (C), for example, the antigen-presenting cells or the antigen-presenting extracellular vesicles described in the present specification may be obtained by the following method.

As soluble proteins, the (A) and (B) (or (D) instead of (A) and (B)) described above, and if necessary. (C) obtained by normal genetic recombination techniques are used, or commercially available products thereof may be used. Next, cells or extracellular vesicles are obtained from desired cells, for example, by a known method, the method described in the present specification, or a method similar thereto. Next, the obtained cells or extracellular vesicles and one or more the soluble proteins described above are reacted in a desired solvent under desired conditions (for example, the method described in JP 2018-104341 A and the like may be referred to). The antigen-presenting cells or the antigen-presenting extracellular vesicles described in the present specification can be obtained by carrying out this operation under appropriately changed conditions until the soluble proteins of (A) and (B) (or (D) instead of (A) and (B)), and if necessary. (C), are contained in the membrane of the extracellular vesicle.

Alternatively, in a case where soluble proteins are used as the proteins of (A) and (B) (or (D) instead of (A) and (B)) described above, and if necessary. (C), for example, the antigen-presenting extracellular vesicles described in the present specification may be obtained by the following method.

As the soluble proteins of (A) and (B) (or (D) instead of (A) and (B)), and if necessary. (C), proteins containing a desired tag added to the N-terminus or C-terminus thereof (examples thereof include a His tag, a FLAG tag, and a PNE tag of SEQ ID NO: 79, and all the tags may be the same tag or different types of tags) are obtained by normal genetic recombination techniques. Next, cells or extracellular vesicles are obtained from the desired cells, for example, by known methods, the methods described in the present specification, or methods similar thereto, and antibodies against these tags or antigen-binding fragments thereof (for example, scFv, Fab, or a nanobody, such as an anti-PNE tag nanobody of SEQ ID NO: 83) and the like are bound to the cells or the extracellular vesicles by a peptide linker or the like, if necessary; alternatively, polynucleotides (for example, SEQ ID NO: 88, 90, and the like) are obtained by normal genetic recombination techniques, the polynucleotides encoding a fusion protein (for example, a fusion protein of SEQ ID NO: 89 of an anti-PNE nanobody (SEQ ID NO: 83), CD8a (SEQ ID NO: 85), and CD81 (SEQ ID NO: 15)) to which an antibody or an antigen-binding fragment thereof (for example, scFv, Fab, or a nanobody) at the N-terminus or C-terminus of a membrane protein capable of being expressed in membrane of a cell or an extracellular vesicle or a transmembrane domain thereof, or the like is bonded, transformed cells (the fusion protein may be a transformed cell that is transiently expressed or a transformed cell (stable strain) that is stably expressed) are obtained by transforming cells using the polynucleotides operably inserted into a vector, the obtained transformed cell are cultured or the like, and cells or extracellular vesicles are recovered by the method described above and the like. The antigen-presenting extracellular vesicles described in the present specification may be obtained by mixing the soluble proteins (A) and (B), and if necessary. (C) to which a tag is added, and extracellular vesicles containing, in membranes thereof, proteins containing antibodies against to the tag or antigen-binding fragments thereof (for example, scFv, Fab, and a nanobody) under predetermined conditions.

Alternatively, the antigen-presenting cells or the antigen-presenting extracellular vesicles described in the specification may be obtained from the transformed cells obtained by performing transformation using a combination of polynucleotides encoding the fusion proteins of (A) to (G) described above.

Alternatively, the antigen-presenting extracellular vesicles described in the present specification may be obtained by a combination of two or more of the methods described above.

The antigen-presenting extracellular vesicles described in the present specification may recognize that the proteins of (A) and (B) (or (D) instead of (A) and (B)), and if necessary, (C) are contained in the membrane by, for example, methods such as flow cytometry. ELISA, and Western blotting.

In an embodiment of the present invention, there is provided a method for preparing the antigen-presenting extracellular vesicles described in the present specification, the method comprising collecting a culture supernatant obtained by culturing the transformed cells described in the present specification.

In an embodiment of the present invention, there is provided a method for preparing the antigen-presenting extracellular vesicles described in the present specification, the method comprising:
simultaneously or sequentially (preferably simultaneously) transforming cells with a single vector or a combination of two or more vectors, the vector comprising,
(i) a polynucleotide encoding the fusion protein or the protein complex of (A) for an antigen-presenting extracellular vesicle described in the present specification.
(ii) a polynucleotide encoding the fusion protein of (B) for an antigen-presenting extracellular vesicle described in the present specification, and optionally,
(iii) a polynucleotide encoding the fusion protein of (C) for an antigen-presenting extracellular vesicle described in the present specification; and
collecting a culture supernatant obtained by culturing the obtained transformed cells.

Alternatively, in an embodiment of the present invention, there is provided a method for preparing the antigen-presenting extracellular vesicles described in the present specification, the method comprising:
simultaneously or sequentially (preferably simultaneously) transforming cells with a single vector or a combination of two or more vectors, the vector comprising,
(iv) a polynucleotide encoding the fusion protein of (D) for an antigen-presenting extracellular vesicle described in the present specification, in which the fusion protein comprises an antigen-presenting MHC molecule and at least one T-cell stimulatory cytokine and is capable of the antigen and the T-cell stimulatory cytokine outside membrane, and optionally,
(iii) a polynucleotide encoding the fusion protein of (C) for an antigen-presenting extracellular vesicle described in the present specification; and
collecting a culture supernatant obtained by culturing the obtained transformed cells.

Alternatively, in an embodiment of the present invention, there is provided a method for preparing the antigen-presenting extracellular vesicles described in the present specification, the method comprising: transforming cells with a vector comprising,
(v) a polynucleotide encoding the fusion protein of (E) for an antigen-presenting extracellular vesicle described in the present specification, in which the fusion protein contains an antigen-presenting MHC molecule, at least one T-cell stimulatory cytokine, and a T-cell costimulatory molecule, and is capable of the antigen and the T-cell stimulatory cytokine outside membrane; and
collecting a culture supernatant obtained by culturing the obtained transformed cells.

In an embodiment of the present invention, a method for preparing the antigen-presenting cells described in the present specification is provided.

In an embodiment of the present invention, there is provided a method for preparing the antigen-presenting cells described in the present specification, the method including:
simultaneously or sequentially (preferably simultaneously) transforming cells with a single vector or a combination of two or more vectors,
the vector comprising,
(i) a polynucleotide encoding the fusion protein or the protein complex of (A) for an antigen-presenting cell described in the present specification,
(ii) a polynucleotide encoding the fusion protein of (B) for an antigen-presenting cell described in the present specification, and optionally,
(iii) a polynucleotide encoding the fusion protein of (C) for an antigen-presenting cell described in the present specification.

Alternatively, in an embodiment of the present invention, there is provided a method for preparing the antigen-presenting cells described in the present specification, the method including:
simultaneously or sequentially (preferably simultaneously) transforming cells with a single vector or a combination of two or more vectors,
the vector comprising,
(iv) a polynucleotide encoding the fusion protein of (D) for an antigen-presenting cell described in the present specification, in which the fusion protein comprises an antigen-presenting MHC molecule and at least one T-cell stimulatory cytokine and is capable of presenting the antigen and the T-cell stimulatory cytokine outside membrane, and optionally,
(iii) a polynucleotide encoding the fusion protein of (C) for an antigen-presenting cell described in the present specification.

Alternatively, in an embodiment of the present invention, there is provided a method for preparing the antigen-presenting extracellular vesicles described in the present specification, the method including:
transforming cells with a vector,
the vector comprising
(v) a polynucleotide encoding the fusion protein of (E) for an antigen-presenting cell described in the present specification, in which the fusion protein comprises an antigen-presenting MHC molecule, at least one T-cell stimulatory cytokine, and a T-cell costimulatory molecule, and is capable of presenting the antigen and the T-cell stimulatory cytokine outside membrane.

In an embodiment of the present invention, antigen-presenting extracellular vesicles obtained from the culture supernatant described in the present specification are provided.

In an embodiment of the present invention, there is provided an antigen-presenting extracellular vesicle obtained by a method including the following:
simultaneously or sequentially (preferably simultaneously) transforming cells with a single vector or a combination of two or more vectors,
the vector comprising,
(i) a polynucleotide encoding the fusion protein or the protein complex of (A) for an antigen-presenting extracellular vesicle described in the present specification,
(ii) a polynucleotide encoding the fusion protein of (B) for an antigen-presenting extracellular vesicle described in the present specification, and optionally,
(iii) a polynucleotide encoding the fusion protein of (C) for an antigen-presenting extracellular vesicle described in the present specification; and
collecting a culture supernatant obtained by culturing the obtained transformed cells.

Alternatively, in an embodiment of the present invention, there is provided an antigen-presenting extracellular vesicle obtained by a method including the following:
simultaneously or sequentially (preferably simultaneously) transforming cells with a single vector or a combination of two or more vectors, the vector comprising,
(iv) a polynucleotide encoding the fusion protein of (D) an antigen-presenting extracellular vesicle described in the present specification, in which the fusion protein comprises an antigen-presenting MHC molecule and at least one T-cell stimulatory cytokine and is capable of the antigen and the T-cell stimulatory cytokine outside membrane, and optionally,
(iii) a polynucleotide encoding the fusion protein of (C) an antigen-presenting extracellular vesicle described in the present specification; and
collecting a culture supernatant obtained by culturing the obtained transformed cells.

Alternatively, in an embodiment of the present invention, there is provided an antigen-presenting extracellular vesicle obtained by a method including the following:
transforming cells with,
(v) a polynucleotide encoding the fusion protein of (E) for an antigen-presenting extracellular vesicle described in the present specification, in which the fusion protein comprises an antigen-presenting MHC molecule, at least one T-cell stimulatory cytokine, and a T-cell costimulatory molecule, and is capable of the antigen and the T-cell stimulatory cytokine outside membrane; and
collecting a culture supernatant obtained by culturing the obtained transformed cells.

In an embodiment of the present invention, there is provided an antigen-presenting cell obtained by a method including the following:
simultaneously or sequentially (preferably simultaneously) transforming cells with a single vector or a combination of two or more vectors,
the vector comprising,
(i) a polynucleotide encoding the fusion protein or the protein complex of (A) for an antigen-presenting cell described in the present specification,
(ii) a polynucleotide encoding the fusion protein of (B) for an antigen-presenting cell described in the present specification, and optionally,
(iii) a polynucleotide encoding the fusion protein of (C) for an antigen-presenting cell described in the present specification.

Alternatively, in an embodiment of the present invention, there is provided an antigen-presenting cell obtained by a method including the following:
simultaneously or sequentially (preferably simultaneously) transforming cells with a single vector or a combination of two or more vectors,
the vector comprising,
(iv) a polynucleotide encoding the fusion protein of (D) for an antigen-presenting cell described in the present specification, in which the fusion protein comprises an antigen-presenting MHC molecule and at least one T-cell stimulatory cytokine and is capable of presenting the antigen and the T-cell stimulatory cytokine outside membrane, and optionally,
(iii) a polynucleotide encoding the fusion protein of (C) for an antigen-presenting cell described in the present specification.

Alternatively, in an embodiment of the present invention, there is provided an antigen-presenting cell obtained by a method including the following:
transforming cells with,
(v) a polynucleotide encoding the fusion protein of (E) for an antigen-presenting cell described in the present specification, in which the fusion protein comprises an antigen-presenting MHC molecule, at least one T-cell stimulatory cytokine, and a T-cell costimulatory molecule, and is capable of presenting the antigen and the T-cell stimulatory cytokine outside membrane.

### Composition and Use

In an embodiment of the present invention, there is provided a composition (for example, a pharmaceutical composition) containing the antigen-presenting cell or the antigen-presenting extracellular vesicle described in the present specification, a polynucleotide and/or a vector comprising the same, and/or a transformed cell and/or a culture supernatant thereof. In an embodiment of the present invention, there is provided a pharmaceutical composition comprising the antigen-presenting cell or the antigen-presenting extracellular vesicle described in the present specification or the culture supernatant described in the present specification.

Examples of the composition (for example, the pharmaceutical composition) described in the present specification comprise, but are not limited to, additives such as an excipient, a lubricant, a binder, a disintegrant, a pH regulator, a solvent, a solubilizing aid, a suspending agent, an isotonicifier, a buffer, an analgesic, a preservative, an antioxidant, a colorant, a sweetener, and a surfactant. Those skilled in the art can appropriately select the types of these additives, the amount of these additives used, and the like depending on the purpose. In a case where the pharmaceutical composition is used, these additives are preferably pharmacologically acceptable carriers. Furthermore, in a case where the composition described in the present specification contains a polynucleotide, it is preferable to contain carriers suitable for a drug delivery (DD) of nucleic acids, although not required, and examples of these carriers include lipid nanoparticles (LNP) and polymers (for example, PEI).

The composition (for example, the pharmaceutical composition) described in the present specification can be formulated into, for example, a tablet, a coated tablet, an orally disintegrating tablet, a chewable agent, a pill, granules, fine granules, a powder, a hard capsule, a soft capsule, a solution (examples thereof include a syrup, an injection, and a lotion), a suspension, an emulsion, a jelly, a patch, an ointment, a cream, an inhalant, a suppository, and the like by a method known per se together with the additives described above. The composition may be an oral agent or a parenteral agent. The formulated composition may further contain other beneficial components (for example, other therapeutically beneficial components) depending on the purpose thereof.

The composition according to an embodiment of the present invention can enhance acquired immunity (cellular immunity and/or humoral immunity) to a specific antigen as shown in test examples, and can be used as a pharmaceutical composition for treating or preventing an infectious disease caused by an infectious pathogen when a peptide derived from an infectious pathogen (pathogenic bacteria, viruses, or the like) is used as an antigen.

In addition, as shown in the test examples, the composition according to an embodiment of the present invention can eliminate infectious pathogens by allowing induction of inflammatory cytokines and activating innate immunity (including mobilizing and activating neutrophils, monocytes, macrophages, and the like to phagocytize pathogenic bacteria), and can be used as a pharmaceutical composition for treating or preventing an infectious disease caused by infectious pathogens.

The antigen-presenting cell or antigen-presenting extracellular vesicle (preferably the antigen-presenting cell or the antigen-presenting extracellular vesicle containing an MHC class I-restricted antigen peptide and an MHC class I molecule in the membrane), the polynucleotide and/or the vector comprising the same, and/or the transformed cell and/or the culture supernatant thereof described in the present specification, or the composition comprising them (for example, the pharmaceutical composition) may be useful for treating or preventing cancer.

Therefore, in an embodiment of the present invention, there are provided, for treating or preventing cancer, the antigen-presenting cell, the antigen-presenting extracellular vesicle, the polynucleotide and/or the vector comprising the polynucleotide, and/or the transformed cell and/or the culture supernatant thereof described in the present specification, or the composition (for example, a pharmaceutical composition) containing them. As shown in the test examples, the antigen-presenting extracellular vesicles and the like according to an embodiment of the present invention can proliferate and activate antigen-specific cytotoxic T cells to be used, and when a tumor-associated antigen peptide is used as an antigen to be used, the proliferated and activated cytotoxic T cells recognize and attack cancer cells, such that the cancer cells can be killed.

In another embodiment of the present invention, there is provided a use of the antigen-presenting extracellular vesicle, the polynucleotide and/or the vector comprising the polynucleotide, and/or the transformed cell and/or the culture supernatant thereof described in the present specification, or the composition (for example, a pharmaceutical composition) comprising them, in the manufacture of a medicament for treating or preventing cancer.

In still another embodiment of the present invention, there is provided a method for treating or preventing cancer, the method including administering an effective amount of the antigen-presenting extracellular vesicle, the polynucleotide and/or the vector comprising the polynucleotide, and/or the transformed cell and/or the culture supernatant thereof described in the present specification, or the composition comprising them to a subject in need thereof.

The cancer includes any solid cancer or blood cancer, and examples thereof include, but are not limited to, small cell lung cancer, non-small cell lung cancer, breast cancer, esophageal cancer, stomach cancer, small intestine cancer, large intestine cancer, colon cancer, rectal cancer, pancreatic cancer, prostate cancer, bone marrow cancer, kidney cancer (including kidney cell cancer), parathyroid cancer, adrenal cancer, ureteral cancer, liver cancer, bile duct cancer, cervical cancer, ovarian cancer (for example, the tissue type thereof is serous gland cancer, mucous gland cancer, clear cell adenocarcinoma cancer, and the like), testicular cancer, bladder cancer, external pudendal cancer, penis cancer, thyroid cancer, head and neck cancer, craniopharyngeal cancer, pharyngeal cancer, tongue cancer, skin cancer, Merkel cell cancer, melanoma (malignant melanoma and the like), epithelial cancer, squamous cell carcinoma, basal cell cancer, childhood cancer, unknown primary cancer, fibrosarcoma, mucosal sarcoma, liposarcoma, chondrosarcoma, osteogenic sarcoma, spinal cord tumor, angiosarcoma, lymphangiosarcoma, lymphangiosarcoma. Kaposi's sarcoma, leiomyosarcoma, rhabdomyosarcoma, synovial tumor, mesothelioma, ewing tumor, seminoma, Wilms tumor, brain tumor, glioma, glioblastoma, astrocytoma, myeloblastoma, meningioma, neuroblastoma, medulloblastoma, retinoblastoma, spinal tumor, malignant lymphoma (for example, non-Hodgkin's lymphoma, Hodgkin's lymphoma, and the like), chronic or acute lymphocytic leukemia, and adult T-cell leukemia.

In an embodiment of the present invention, immune checkpoint inhibitors can be used in combination to treat or prevent cancer. The immune checkpoint inhibitors may be administered simultaneously or sequentially to a patient, or may be contained in the pharmaceutical according to the present invention.

Examples of the immune checkpoint inhibitor include, but are not limited to, a PD-1 inhibitor (for example, an anti-PD-1 antibody such as nivolumab or pembrolizumab), a CTLA-4 inhibitor (for example, an anti-CTLA-4 antibody such as ipilimumab), and a PD-L1 inhibitor (for example, an anti-PD-L1 antibody such as durvalumab, atezolizumab, or avelumab). In a case where the immune checkpoint inhibitor is an antibody or an active fragment thereof, the antibody or the active fragment thereof may be bound to a membrane protein capable of being localized onto membrane of an extracellular vesicle or a transmembrane domain thereof or a protein capable of binding to membrane of an extracellular vesicle or a membrane-binding domain thereof to be present on the membrane of the extracellular vesicle according to the present invention.

A combination of these immune checkpoint inhibitors enhances cytotoxicity against cancer cells.

The antigen-presenting cell or the antigen-presenting extracellular vesicle (preferably the antigen-presenting cell or the antigen-presenting extracellular vesicle containing an MHC class II-restricted antigen peptide and an MHC class II molecule in the membrane), the polynucleotide and/or the vector comprising the same, and/or the transformed cell and/or the culture supernatant thereof described in the present specification, or the composition comprising them may be useful for treating or preventing an autoimmune disease. As exemplified in the test examples, the antigen-presenting extracellular vesicles according to an embodiment of the present invention can proliferate and activate antigen-specific regulatory T cells (Treg) to be used, and when an auto-antigen peptide is used as an antigen to be used, the proliferated and activated Treg induces tolerance to the auto-antigen, such that the autoimmune disease can be treated or prevented.

Therefore, in an embodiment of the present invention, there are provided, for treating or preventing an autoimmune disease, the antigen-presenting cell, the antigen-presenting extracellular vesicle, the polynucleotide and/or the vector comprising the polynucleotide, and/or the transformed cell and/or the culture supernatant thereof described in the present specification, or the composition (for example, a pharmaceutical composition) comprising them.

In another embodiment of the present invention, there is provided use of the antigen-presenting cell, the antigen-presenting extracellular vesicle, the polynucleotide and/or the vector comprising the polynucleotide, and/or the transformed cell and/or the culture supernatant thereof described in the present specification, or the composition (for example, a pharmaceutical composition) containing them, for producing a pharmaceutical for treating or preventing an autoimmune disease.

In still another embodiment of the present invention, there is provided a method for treating or preventing an autoimmune disease, the method including administering an effective amount of the antigen-presenting cell, the antigen-presenting extracellular vesicle, the polynucleotide and/or the vector comprising the polynucleotide, and/or the transformed cell and/or the culture supernatant thereof described in the present specification, or the composition comprising them to a subject who requires them.

Examples of the autoimmune disease include, but are not limited to, asthma, psoriasis, systemic erythematosus, Guillain-Barre syndrome. Sjogren's syndrome, multiple sclerosis, myasthenia gravis, malignant anemia, Basedow's disease, Hashimoto thyroiditis, type I diabetes, Crohn's disease, inflammatory bowel disease, and rheumatoid arthritis.

The antigen-presenting cell and the antigen-presenting extracellular vesicle (preferably the antigen-presenting cell and the antigen-presenting extracellular vesicle containing an MHC class II-restricted antigen peptide and an MHC class II molecule on the membrane), the polynucleotide and/or the vector comprising the same, and/or the transformed cell and/or the culture supernatant thereof described in the present specification, or the composition comprising them (for example, the pharmaceutical composition) may be useful for treating or preventing an allergic disease. As shown in the test examples, the antigen-presenting extracellular vesicles according to an embodiment of the present invention can proliferate and activate antigen-specific regulatory T cells (Treg) to be used, and when an allergen is used as an antigen to be used, the proliferated and activated Treg induces tolerance to the allergen, such that the allergic disease can be treated or prevented.

Therefore, in an embodiment of the present invention, there are provided, for treating or preventing an allergic disease, the antigen-presenting cell, the antigen-presenting extracellular vesicle, the polynucleotide and/or the vector comprising the polynucleotide, and/or the transformed cell and/or the culture supernatant thereof described in the present specification, or the composition (for example, a pharmaceutical composition) containing them.

In another embodiment of the present invention, there is provided use of the antigen-presenting cell, the antigen-presenting extracellular vesicle, the polynucleotide and/or the vector comprising the polynucleotide, and/or the transformed cell and/or the culture supernatant thereof described in the present specification, or the composition (for example, a pharmaceutical composition) containing them, for producing a pharmaceutical for treating or preventing an allergic disease.

In still another embodiment of the present invention, there is provided a method for treating or preventing an allergic disease, the method including administering an effective amount of the antigen-presenting cell, the antigen-presenting extracellular vesicle, the polynucleotide and/or the vector comprising the polynucleotide, and/or the transformed cell and/or the culture supernatant thereof described in the present specification, or the composition comprising them to a subject who requires them.

Examples of the allergic disease include, but are not limited to, allergic rhinitis, atopic dermatitis, allergic asthma, allergic conjunctivitis, allergic gastro-enteritis, food allergies, drug allergies, and urticaria.

Examples of the subject to be treated or prevented from the various diseases described above include, but are not limited to, animals such as mammals, for example, rodents such as a mouse, a rat, a hamster, and a guinea pig; lagomorph such as a rabbit; ungulates such as a pig, a cow, a goat, a horse, and a sheep; carnivora such as a dog and a cat; and primates such as a human, a monkey, a rhesus monkey, a crab-eating macaque, a marmoset, an orangutan, and a chimpanzee; and plants. The subject is preferably an animal, more preferably a rodent or a primate, and sill more preferably a mouse or a human.

A dosage of a formulation obtained by formulating the antigen-presenting cell, the antigen-presenting extracellular vesicle, the polynucleotide and/or the vector comprising the polynucleotide, and/or the transformed cell and/or the culture supernatant thereof described in the present specification, or the composition comprising them can be appropriately determined in consideration of a gender, an age, a weight, a health status, a degree of medical condition, or a diet of a subject to be administered, an administration time, an administration method, a combination with other drugs, and other factors.

Method for Activating, Proliferating, and/or Differentiating T Cells Against Specific Antigen

The antigen-presenting cells or the antigen-presenting extracellular vesicles described in the present specification can activate, proliferate, and differentiate T cells against a specific antigen by contacting with the T cells (although not limited thereto, for example, T cells or T cell populations obtained from peripheral blood, spleen, and the like) in vitro, ex vivo, and/or in vivo.

In an embodiment of the present invention, there is provided a method for activating, proliferating, and/or differentiating T cells against a specific antigen, the method comprising bringing the antigen-presenting cells or the antigen-presenting extracellular vesicles described in the present specification into contact with T cells in vitro or ex vivo.

In an embodiment of the present invention, there are provided T cells obtained by the method described above.

The T cells obtained by the method described above may be administered to a subject in order to treat and/or prevent a disease (for example, cancer, an autoimmune disease, an allergic disease, or the like).

### EXAMPLES

Hereinafter, the present invention will be described in more detail with reference to examples, and these examples do not limit the scope of the present invention at all.

### Plasmid Preparation 1:

A vector for expressing, on membrane of an extracellular vesicle, an MHC class I molecule capable of presenting an antigen outside membrane was prepared using a pCAG-puro vector.

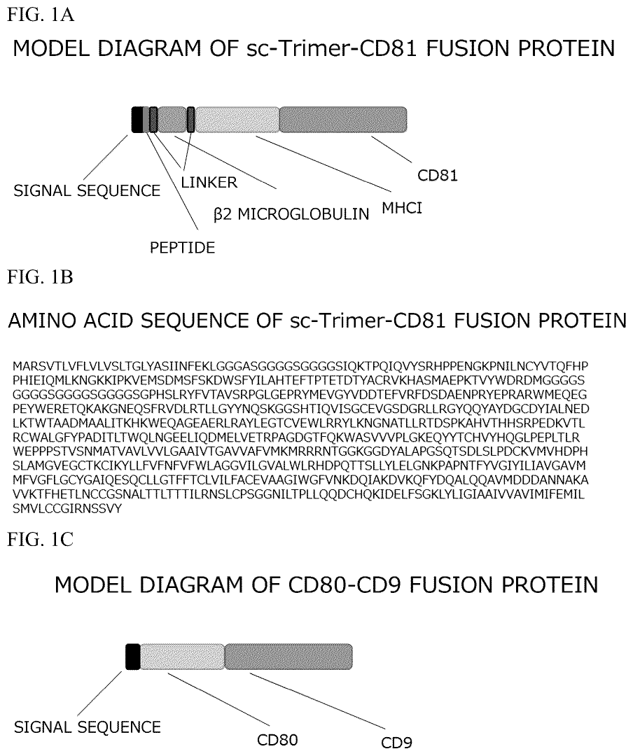

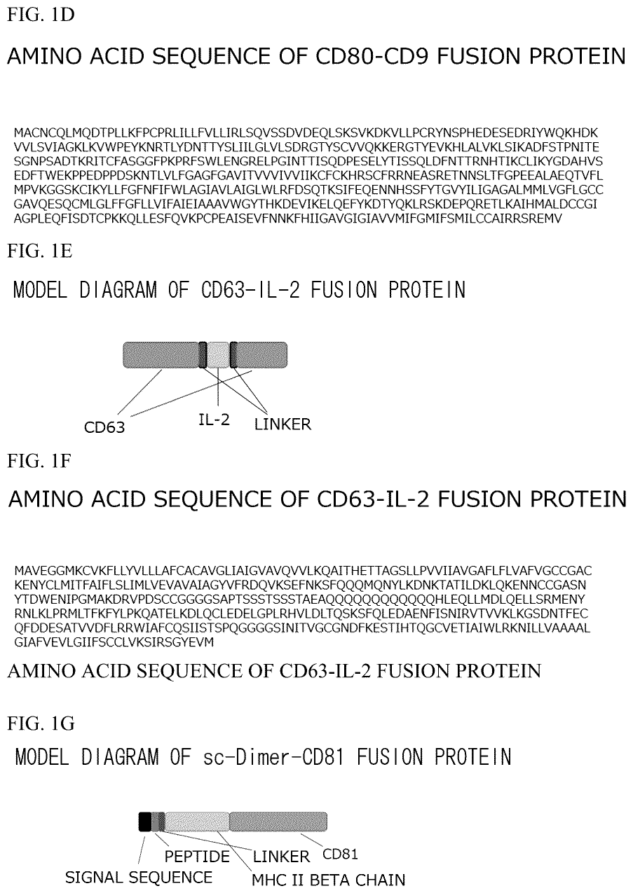

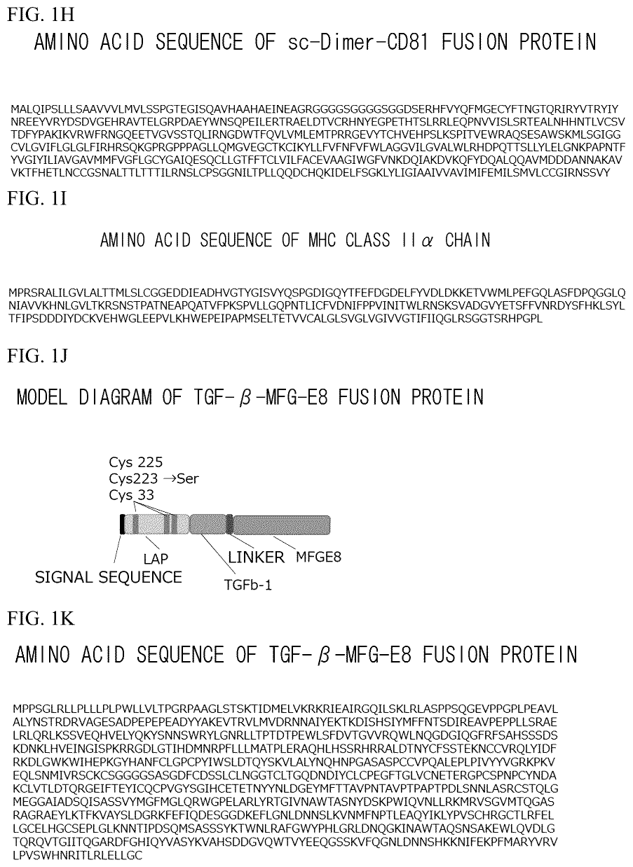

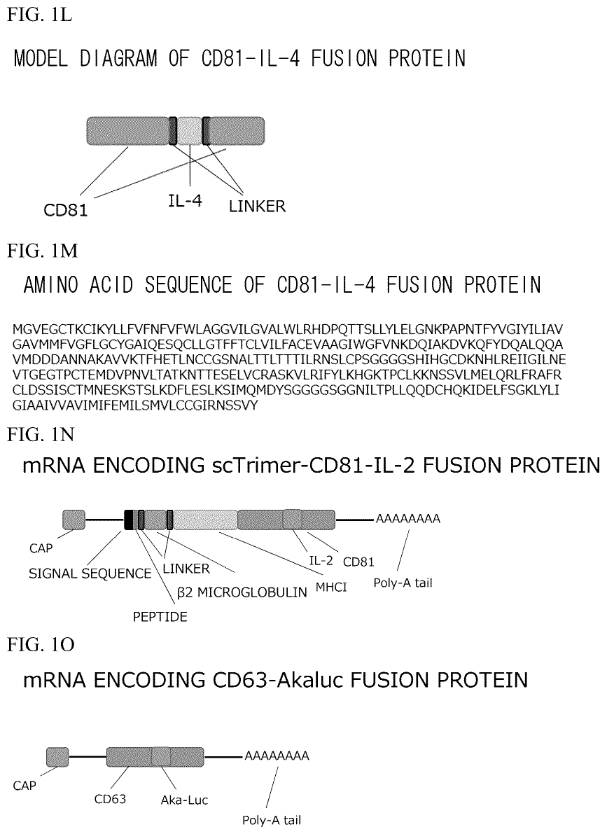

With established cloning techniques, a single chain trimer (sc-Trimer) consisting of a polynucleotide (SEQ ID NO: 2) encoding a signal peptide (amino acids 1 to 20; SEQ ID NO: 1) of β₂ microglobulin, a polynucleotide (SEQ ID NO: 4) encoding an OVA peptide (SEQ ID NO: 3) as a model antigen peptide, a peptide linker (amino acid sequence: SEQ ID NO: 5, polynucleotide: SEQ ID NO: 6), a polynucleotide (SEQ ID NO: 8) encoding a full-length sequence (amino acids 21 to 119; SEQ ID NO: 7) of β₂ microglobulin from which a signal peptide was removed, a polynucleotide (SEQ ID NO: 12) encoding a peptide linker (SEQ ID NO: 11), and a polynucleotide (SEQ ID NO: 10) encoding a full-length sequence (amino acids 22 to 369; SEQ ID NO: 9) of an MHC class Iα chain from which a signal peptide was removed was prepared (amino acid sequence: SEQ ID NO: 13; polynucleotide: SEQ ID NO: 14). Next, a polynucleotide (SEQ ID NO: 18; corresponding amino acid sequence: SEQ ID NO: 17) in which a sc-Trimer was linked to a polynucleotide (SEQ ID NO: 16) encoding a full-length sequence (amino acids 1 to 236; SEQ ID NO: 15) of CD81 as a Tetraspanin was inserted into the pCAG-puro vector (FIGS. 1A and 1B: hereinafter, sc-Trimer-CD81).

With the same method, in order to express CD80 as one of T-cell costimulatory molecules on membrane of an extracellular vesicle, a polynucleotide (SEQ ID NO: 24: corresponding amino acid sequence: SEQ ID NO: 23) in which a polynucleotide (SEQ ID NO: 20) encoding a full-length sequence (amino acids 1 to 306; SEQ ID NO: 19) of CD80 was linked to a polynucleotide (SEQ ID NO: 22) encoding a full-length sequence (amino acids 1 to 306; SEQ ID NO: 21) of CD9 as a Tetraspanin was inserted into a pCAG-puro or pMX vector (FIGS. 1C and 1D: hereinafter, CD80-CD9).

With the same method, in order to express IL-2 as one of T-cell stimulatory cytokines on membrane of an extracellular vesicle, a polynucleotide (SEQ ID NO: 26) encoding a full-length sequence (amino acids 21 to 169; SEQ ID NO: 25) from which a single peptide of IL-2 was removed was inserted between the amino acids 170C and 1711 in a large extracellular loop of a mouse CD63 (amino acids 1 to 238; SEQ ID NO: 27; polynucleotide: SEQ ID NO: 28) as a Tetraspanin (that is, a sequence of IL-2 was inserted between a polynucleotide (SEQ ID NO: 58) encoding a partial sequence of CD63 of SEQ ID NO: 57 and a polynucleotide (SEQ ID NO: 60) encoding a partial sequence of CD63 of SEQ ID NO: 59). Note that polynucleotides (SEQ ID NO: 30) encoding a peptide linker (amino acid sequence GGGGS: SEQ ID NO: 29) were added to the N-terminus and the C-terminus of IL-2, respectively. The polynucleotide (SEQ ID NO: 32; corresponding amino acid sequence: SEQ ID NO: 31) was inserted into the pCAG-puro vector (FIGS. 1E and 1F: hereinafter, CD63-IL-2).

### Plasmid Preparation 2:

A vector for expressing, on membrane of an extracellular vesicle, an MHC class II molecule capable of presenting an antigen outside membrane was prepared using a pCAG-puro vector.

With established cloning techniques, a single chain dimer (sc-Dimer) in which a polynucleotide (SEQ ID NO: 34) encoding a signal peptide (amino acids 1 to 27: SEQ ID NO: 33) of an MHC class IIβ chain, a polynucleotide (SEQ ID NO: 36) encoding an OVA peptide (SEQ ID NO: 35) as a model antigen peptide, and a polynucleotide (SEQ ID NO: 38) encoding a full-length sequence (amino acids 28 to 265; SEQ ID NO: 37) of an MHC class IIβ chain from which a signal peptide was removed were linked by a polynucleotide (SEQ ID NO: 40) encoding a peptide linker (SEQ ID NO: 39) was prepared (amino acid sequence: SEQ ID NO: 41; polynucleotide: SEQ ID NO: 42). Next, a polynucleotide (SEQ ID NO: 44; corresponding amino acid sequence: SEQ ID NO: 43) in which a sc-Dimer was linked to a polynucleotide (SEQ ID NO: 16) encoding a full-length sequence (amino acids 1 to 236: SEQ ID NO: 15) of CD81 as a Tetraspanin was inserted into the pCAG-puro vector (FIGS. 1G and 1H: hereinafter, sc-Dimer-CD81).

A polynucleotide (SEQ ID NO: 46) encoding a full-length sequence (amino acids 1 to 256; SEQ ID NO: 45) of an MHC class IIα chain as a constituent element of an MHC class II molecule was inserted into another pCAG-puro vector (FIG. 1I: hereinafter, an MHC class IIα chain).

With the same method, in order to express TGF-β₁ as one of T-cell stimulatory cytokines on membrane of an extracellular vesicle, a polynucleotide (SEQ ID NO: 48) encoding a full-length sequence (amino acids 1 to 390; SEQ ID NO: 47) of TGF-β₁ in which three 33^{rd}, 223^{rd}, and 225^{th} C's of a LAP domain were changed to S's and a polynucleotide (SEQ ID NO: 50) encoding a full-length sequence (amino acids 23 to 463; SEQ ID NO: 49) from which a signal peptide of MFG-E8 in which 89^{th} D was changed to E was removed were linked by a polynucleotide (SEQ ID NO: 30) encoding a peptide linker (SEQ ID NO: 29). The polynucleotide (SEQ ID NO: 52; corresponding amino acid sequence: SEQ ID NO: 51) was inserted into the pCAG-puro vector (FIGS. 1J and 1K: hereinafter, TGF-β-MFG-E8). 30 With the same method, in order to express IL-4 as one of T-cell stimulatory cytokines on membrane of an extracellular vesicle, a polynucleotide (SEQ ID NO: 54) encoding a full-length sequence (amino acids 21 to 140; SEQ ID NO: 53) from which a single peptide of IL-4 was removed was inserted between the amino acids 177S and 178G in a large extracellular loop of a mouse CD81 (amino acids 1 to 236; SEQ ID NO: 15; polynucleotide: SEQ ID NO: 16) as a Tetraspanin (that is, a sequence of IL-4 was inserted between a polynucleotide (SEQ ID NO: 62) encoding a partial sequence of CD81 of SEQ ID NO: 61 and a polynucleotide (SEQ ID NO: 64) encoding a partial sequence of CD81 of SEQ ID NO: 63). Note that polynucleotides (SEQ ID NO: 30) encoding a peptide linker (amino acid sequence GGGGS; SEQ ID NO: 29) were added to the N-terminus and the C-terminus of IL-4, respectively. The polynucleotide (SEQ ID NO: 56; corresponding amino acid sequence: SEQ ID NO: 55) was inserted into the pCAG-puro vector (FIGS. 1L and 1M: hereinafter, CD81-IL-4).

### Plasmid Preparation 3:

sc-Dimer-CD81-IL-12p40

At the sc-Dimer, a polynucleotide (SEQ ID NO: 92) encoding a protein (SEQ ID NO: 91) obtained by fusing CD81 to IL-12p40 as a subunit of IL-12 as a T-cell stimulatory cytokine was inserted into a pCAG-puro vector, thereby preparing a vector expressing a fusion protein.

### IL-12p35

A polynucleotide (SEQ ID NO: 98) encoding IL-12p35 (SEQ ID NO: 97) as one subunit of IL-12 was inserted into a pCAG-puro or pMX vector to prepare a vector expressing IL-12p35.

### CD81-IL-6

In order to express IL-6 as one of T-cell stimulatory cytokines on membrane of an extracellular vesicle, a polynucleotide (SEQ ID NO: 100) encoding a full-length sequence (SEQ ID NO: 99) from which a signal peptide of IL-6 was removed was introduced into a polynucleotide encoding an extracellular loop of CD81 as a Tetraspanin, and a polynucleotide (SEQ ID NO: 102) encoding a CD81-IL-6 fusion protein (SEQ ID NO: 101) was inserted into a pCAG-puro or pMX vector, thereby preparing a vector expressing a fusion protein.

hCD80-hCD9

In order to express human CD80 as one of T-cell costimulatory molecules on membrane of an extracellular vesicle, a polynucleotide (SEQ ID NO: 108) encoding a fusion protein (SEQ ID NO: 107) of human CD80 and human CD9 as a Tetraspanin was inserted into a pCAG-puro or pMX vector, thereby preparing a vector expressing a fusion protein.

sc-Trimer-CD81-IL-2

In order to express IL-2 as one of T-cell stimulatory cytokines on membrane of an extracellular vesicle, similar to the CD81-IL-4, a polynucleotide encoding a fusion peptide of CD81-IL2 was prepared, a sequence of the polynucleotide was linked to a nucleotide encoding a sc-Trimer-, and a polynucleotide (SEQ ID NO: 136) encoding sc-Trimer-CD81-IL-2 (SEQ ID NO: 135) was prepared and inserted into a pCAG-puro or pMX vector, thereby preparing a vector expressing a fusion protein.

hsc-Trimer-hCD81

Using the sc-Trimer-CD81 as a human gene sequence (using HLA-A2402 as a sequence of MHC-I), a polynucleotide (SEQ ID NO: 132) encoding hsc-Trimer-hCD81 (SEQ ID NO: 131) was prepared and inserted into a pCAG-puro or pMX vector to prepare a vector expressing a fusion protein.

### SARS-COV2sc-Trimer-hCD81

Using a SARS-COV-2 peptide (amino acid sequence: SEQ ID NO: 141; polynucleotide: SEQ ID NO: 142) as an antigen and HLA-A0201 as an MHC molecule, a polynucleotide (SEQ ID NO: 148) encoding an antigen-presenting MHC molecule (SARS-COV2sc-Trimer; amino acid sequence: SEQ ID NO: 147) was prepared and was further linked to a polynucleotide encoding hCD81, thereby preparing a polynucleotide (SEQ ID NO: 150) encoding SARS-COV2sc-Trimer-hCD81 (SEQ ID NO: 149). The prepared polynucleotide was inserted into a pCAG-puro or pMX vector to prepare a vector expressing a fusion protein.

hCD63-hIL-2

The CD63-IL-2 was prepared using a human gene sequence. A polynucleotide (SEQ ID NO: 116) encoding hCD63-hIL-2 (SEQ ID NO: 115) was prepared and inserted into a pCAG-puro or pMX vector to prepare a vector expressing a fusion protein.

### CD63-Akaluc

As a negative control, CD63 and Akaluc luciferase were fused to prepare a polynucleotide (SEQ ID NO: 140) for localizing an AlkaLuc fusion protein (SEQ ID NO: 139) to an extracellular vesicle, and the polynucleotide was inserted into a pCAG-puro or pMX vector, thereby preparing a vector expressing a fusion protein.

### Plasmid Preparation 4:

### HLADR-1sc-TPI1-hCD81

A signal sequence of an HLA DR1β chain (amino acid sequence: SEQ ID NO: 151; polynucleotide sequence: No. 152), a sequence of a TPI-1 peptide (amino acid sequence: SEQ ID NO: 153; polynucleotide sequence: No. 154), and a sequence of an HLA DR1β chain (amino acid sequence: SEQ ID NO: 155; polynucleotide sequence: No. 156) were bonded to prepare a sequence encoding an HLA DR1β chain presenting a TPI-1 peptide. A sequence of hCD81 (amino acid sequence: SEQ ID NO: 159; polynucleotide sequence: No. 160) was connected to this sequence. Furthermore, a sequence of an HLA DR1α chain (amino acid sequence: SEQ ID NO: 163; polynucleotide sequence: No. 164) was bonded by a P2A sequence (amino acid sequence: SEQ ID NO: 161; polynucleotide sequence: No. 162) to prepare a polynucleotide for presenting a TPI-1 peptide outside membrane of an extracellular vesicle. The prepared polynucleotide was inserted into a pCAG-puro or pMX vector to prepare a vector expressing a fusion protein HLADR-1sc-TPI1-hCD81 (amino acid sequence: SEQ ID NO: 165; polynucleotide sequence: No. 166). From mRNA transcribed from such a sequence, a fusion protein of an HLA DR1β chain presenting a TPI-1 peptide and hCD81 and an HLA DR1α chain are translated by the action of P2A, a 2A peptide, and there is an MHC molecule presenting the TPI-1 peptide on membrane of an extracellular vesicle by binding them.

hIL-12sc-MFGe8

A sequence encoding an IL-12β subunit (amino acid sequence: SEQ ID NO: 171; polynucleotide sequence: No. 172) was linked to a sequence encoding an IL-12β subunit (amino acid sequence: SEQ ID NO: 167; polynucleotide sequence: No. 167) by a sequence of a linker (amino acid sequence: SEQ ID NO: 169; polynucleotide sequence: No. 170) to prepare a sequence encoding IL-12, and a sequence of MFGe8 (amino acid sequence: SEQ ID NO: 175; polynucleotide sequence: No. 176) was linked to the sequence by a sequence of a linker (amino acid sequence: SEQ ID NO: 173; polynucleotide sequence: No. 174) to prepare a polynucleotide for expressing IL-12 in an extracellular vesicle. The prepared polynucleotide was inserted into a pCAG-puro or pMX vector to prepare a vector expressing a fusion protein hIL-12sc-MFGe8 (amino acid sequence: SEQ ID NO: 177; polynucleotide sequence: No. 178).

### Fusion Protein of TPI-1 Peptide-Specific TCR and Venus

A sequence of a TPI-specific TCRβ chain (amino acid sequence: SEQ ID NO: 179; polynucleotide sequence: No. 180) was linked to a sequence of TCRα (amino acid sequence SEQ ID NO: 183; polynucleotide sequence: No. 184) by a sequence of P2A (amino acid sequence SEQ ID NO: 181; polynucleotide sequence: No. 182), and a sequence of a Venus fluorescent protein (amino acid sequence SEQ ID NO: 187; polynucleotide sequence: No. 188) was further linked to a 3 end side thereof by a sequence of P2A (amino acid sequence SEQ ID NO: 185; polynucleotide sequence: No. 186), thereby preparing a polynucleotide encoding a fusion protein (amino acid sequence SEQ ID NO: 189; polynucleotide sequence: No. 190) of TPI-1 peptide-specific TCR and Venus. The prepared polynucleotide was inserted into a pCAG-puro or pMX vector to prepare a vector expressing a fusion protein of TPI-1 peptide-specific TCR and Venus.

### Plasmid Preparation 5:

sc-Trimer-T2A-IL-2-CD8-P2A-CD80

Using a pET-15b vector, a vector for preparing mRNA for expressing an antigen-presenting MHC class I molecule, IL-2, and CD80 was prepared on membrane of a cell.

A sequence of sc-Trimer (amino acid sequence: SEQ ID NO: 191; polynucleotide sequence: SEQ ID NO: 192) prepared in the same manner as described above:
a sequence of T2A (amino acid sequence: SEQ ID NO: 193, polynucleotide sequence: SEQ ID NO: 194):
a sequence of encoding a fusion protein of IL-2 (amino acid sequence: SEQ ID NO: 195, polynucleotide: SEQ ID NO: 196), a linker sequence (amino acid sequence: SEQ ID NO: 197, polynucleotide: SEQ ID NO: 198), and a partial sequence of CD8 (containing a transmembrane domain: amino acid sequence: SEQ ID NO: 199, polynucleotide: SEQ ID NO: 200), which is a fusion protein for expressing IL-2 on membrane of a cell:
a sequence of P2A (amino acid sequence: SEQ ID NO: 201, polynucleotide sequence: SEQ ID NO: 202); and
a sequence encoding CD80 (amino acid sequence: SEQ ID NO: 203, polynucleotide sequence: SEQ ID NO: 204) were linked to prepare a sequence encoding sc-Trimer-T2A-IL-2-CD8-P2A-CD80 (amino acid sequence: SEQ ID NO: 205, polynucleotide sequence: SEQ ID NO: 206) (FIG. 1P). From mRNA transcribed from such a sequence, a membrane protein containing sc-Trimer, a membrane protein containing IL-2-CD8, and a membrane protein containing CD80 are translated by the action of the 2A peptides T2A and P2A, and are present on the cell membrane of one cell.

As a control, a plasmid transcribing mRNA encoding CD81 (amino acid sequence: SEQ ID NO: 207, polynucleotide sequence: SEQ ID NO: 208); and a plasmid transcribing mRNA encoding a full-length of OVA (amino acid sequence: SEQ ID NO: 209, polynucleotide sequence: SEQ ID NO: 210) were prepared.

### Plasmid Preparation 6:

sc-Trimer-T2A-IL-15sa-P2A-CD80

An expression vector for preparing an antigen-presenting MHC class I molecule, an IL-15 superagonist (hereinafter, IL-15sa: a complex of IL-15 and a sushi domain of an IL-15 receptor), and mRNA for expressing CD80 was prepared on membrane of a cell.

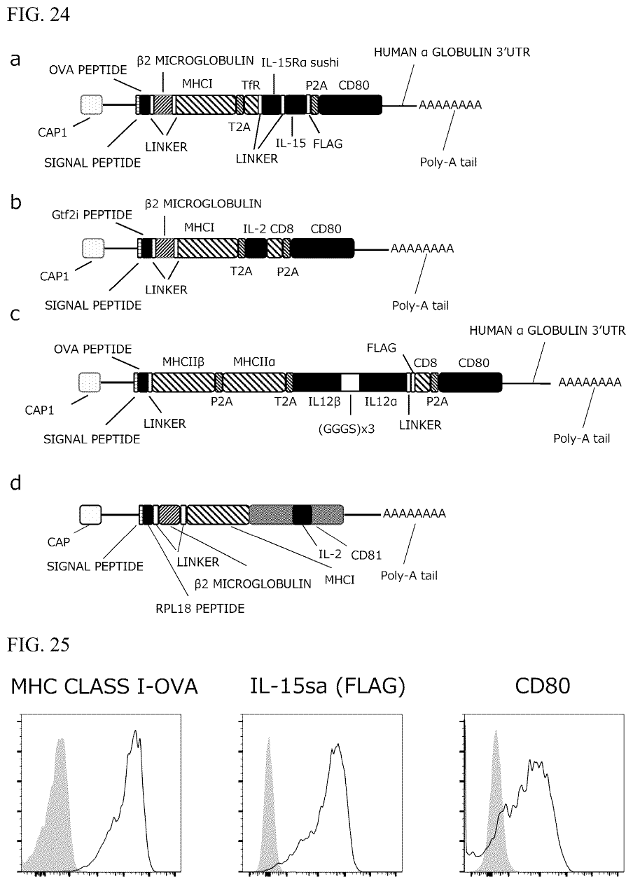

In the same manner as described above, from an N-terminus,
a signal peptide of β₂ microglobulin (amino acid sequence: SEQ ID NO: 215; polynucleotide sequence: SEQ ID NO: 216):
an OVA peptide (amino acid sequence: SEQ ID NO: 217; polynucleotide sequence: SEQ ID NO: 218):
a linker (amino acid sequence: SEQ ID NO: 219; polynucleotide sequence: SEQ ID NO: 220):
β₂ microglobulin from which a signal peptide was removed (amino acid sequence: SEQ ID NO: 221; polynucleotide sequence: SEQ ID NO: 222);
a linker (amino acid sequence: SEQ ID NO: 223; polynucleotide sequence: SEQ ID NO: 224);
an MHC class Iα chain (from which a signal peptide was removed) (amino acid sequence: SEQ ID NO: 225; polynucleotide sequence: SEQ ID NO: 226);
a sequence of T2A (amino acid sequence: SEQ ID NO: 227; polynucleotide sequence: SEQ ID NO: 228):
an N-terminal peptide (amino acid sequence: SEQ ID NO: 229; polynucleotide sequence: SEQ ID NO: 230) of TfR (transferrin receptor 1):
a linker (amino acid sequence: SEQ ID NO: 231; polynucleotide sequence: SEQ ID NO: 232):
an IL-15Rα sushi domain (amino acid sequence: SEQ ID NO: 233; polynucleotide sequence: SEQ ID NO: 234):
a linker (amino acid sequence: SEQ ID NO: 235; polynucleotide sequence: SEQ ID NO: 236):
IL-15 (amino acid sequence: SEQ ID NO: 237; polynucleotide sequence: SEQ ID NO: 238):
FLAG (amino acid sequence: SEQ ID NO: 239; polynucleotide sequence: SEQ ID NO: 240);
a sequence of P2A (amino acid sequence: SEQ ID NO: 241; polynucleotide sequence: SEQ ID NO: 242); and
CD80 (amino acid sequence: SEQ ID NO: 243; polynucleotide sequence: SEQ ID NO: 244) were linked, in this order, thereby preparing a sequence encoding sc-Trimer-T2A-IL-15sa-P2A-CD80 (amino acid sequence: SEQ ID NO: 245, polynucleotide sequence: SEQ ID NO: 246) (FIG. 24(a)). From mRNA transcribed from such a sequence, a membrane protein containing sc-Trimer, a membrane protein containing IL-15sa (IL-15sa fused to the C-terminus is presented on the cell membrane surface by the action of TfR, a type 2 transmembrane protein), and a membrane protein containing CD80 are translated by the action of the 2A peptides T2A and P2A, and are present on the cell membrane of one cell.

### Plasmid Preparation 7:

A sequence encoding sc-Trimer (Gtf2i)-T2A-IL-2-CD8-P2A-CD80 presenting a Gtf2i peptide (amino acid sequence: SEQ ID NO: 279, polynucleotide sequence: SEQ ID NO: 280; Non Patent Literature 3), a neoantigen (cancer antigen) instead of an OVA peptide, was prepared (amino acid sequence: SEQ ID NO: 281, polynucleotide sequence: SEQ ID NO: 282) (FIG. 24(b)).

### Plasmid Preparation 8:

### OVAp-MHCIIβ-P2A-MHCIIα-T2A-IL-12sc-CD8-P2A-CD80

An expression vector for preparing mRNA for expressing an antigen-presenting MHC class II molecule, IL-12sc, and CD80 was prepared on membrane of a cell.

In the same manner as described above, from an N-terminus,
a signal peptide of an MHC class IIβ chain (amino acid sequence: SEQ ID NO: 247; polynucleotide sequence: SEQ ID NO: 248);
an OVA peptide (amino acid sequence: SEQ ID NO: 249; polynucleotide sequence: SEQ ID NO: 250);
a linker (amino acid sequence: SEQ ID NO: 251; polynucleotide sequence: SEQ ID NO: 252):
an MHC class IIβ chain (from which a signal peptide was removed) (amino acid sequence: SEQ ID NO: 253; polynucleotide sequence: SEQ ID NO: 254);
P2A (amino acid sequence: SEQ ID NO: 255; polynucleotide sequence: SEQ ID NO: 256);
an MHC class IIα chain (from which a signal peptide was removed) (amino acid sequence: SEQ ID NO: 257; polynucleotide sequence: SEQ ID NO: 258);
T2A (amino acid sequence: SEQ ID NO: 259; polynucleotide sequence: SEQ ID NO: 260);
IL-12β (amino acid sequence: SEQ ID NO: 261; polynucleotide sequence: SEQ ID NO: 262);
a linker (amino acid sequence: SEQ ID NO: 263; polynucleotide sequence: SEQ ID NO: 264);
IL-12a (amino acid sequence: SEQ ID NO: 265; polynucleotide sequence: SEQ ID NO: 266);
a linker (amino acid sequence: SEQ ID NO: 267; polynucleotide sequence: SEQ ID NO: 268);
FLAG (amino acid sequence: SEQ ID NO: 269; polynucleotide sequence: SEQ ID NO: 270);
a transmembrane domain of CD8 (amino acid sequence: SEQ ID NO: 271; polynucleotide sequence: SEQ ID NO: 272);
P2A (amino acid sequence: SEQ ID NO: 273; polynucleotide sequence: SEQ ID NO: 274); and
CD80 (amino acid sequence: SEQ ID NO: 275; polynucleotide sequence: SEQ ID NO: 276) were linked, in this order, thereby preparing a sequence encoding OVAp-MHCIIβ-P2A-MHCIIα-T2A-IL-12sc-CD8-P2A-CD80 (amino acid sequence: SEQ ID NO: 277, polynucleotide sequence: SEQ ID NO: 278) (FIG. 24(c)). From mRNA transcribed from such a sequence, a membrane protein capable of presenting an OVA peptide antigen outside membrane, the membrane protein containing an MHC class II molecule and IL-12, and a membrane protein containing CD80 are translated by the action of the 2A peptides T2A and P2A, and are present on the cell membrane of one cell.

### Plasmid Preparation 9:

A sequence encoding sc-Trimer (RPL18 peptide)-CD81-IL-2 was prepared by introducing an RPL18 peptide (amino acid sequence: SEQ ID NO: 283, polynucleotide sequence: SEQ ID NO: 284; Non Patent Literature 3), a neoantigen (cancer antigen), instead of OVA peptide (FIG. 24(d)).

Table elective sequences used in examples are shown in Tables 1 to 21. Note that the underline portion in each sequence indicates a signal peptide.

TABLE 1

| | | SEQ |
|---|---|---|
| | Sequence | ID NO: |
| Signal peptide | MARSVTLVFLVLVSLTGLYA | 1 |
| of ß₂ | ATGGCTCGCTCGGTGACCCTGGTCTTTCTGGTGCTTGTCTCACTGACCGGCCTGTAT | 2 |
| microglobulin | GCT | |
| OVA | SIINFEKL | 3 |
| peptide 1 | TCCATTATAAATTTTGAAAAGTTG | 4 |
| (for MHC class | | |
| I molecule) | | |
| Peptide linker 1 | GGGASGGGGSGGGGS | 5 |
| | GGCGGAGGTGCCTCTGGCGGTGGGGGCAGCGGTGGAGGGGGCAGT | 6 |
| ß₂ | IQKTPQIQVYSRHPPENGKPNILNCYVTQFHPPHIEIQMLKNGKKIPKVEMSDMSFS | 7 |
| Microglobulin | KDWSFYILAHTEFTPTETDTYACRVKHASMAEPKTVYWDRDM | |
| (from which | ATCCAGAAAACCCCTCAAATTCAAGTATACTCACGCCACCCACCGGAGAATGGGAAG | 8 |
| signal peptide | CCGAACATACTGAACTGCTACGTAACACAGTTCCACCCGCCTCACATTGAAATCCAA | |
| is removed) | ATGCTGAAGAACGGGAAAAAAATTCCTAAAGTAGAGATGTCAGATATGTCCTTCAGC | |
| | AAGGACTGGTCTTTCTATATCCTGGCTCACACTGAATTCACCCCCACTGAGACTGAT | |
| | ACATACGCCTGCAGAGTTAAGCATGCCAGTATGGCCGAGCCCAAGACCGTCTACTGG | |
| | GATCGAGACATG | |
| MHC class | GPHSLRYFVTAVSRPGLGEPRYMEVGYVDDTEFVRFDSDAENPRYEPRARWMEQEGP | 9 |
| Iα chain | EYWERETQKAKGNEQSFRVDLRTLLGYYNQSKGGSHTIQVISGCEVGSDGRLLRGYQ | |
| (from which | QYAYDGCDYIALNEDLKTWTAADMAALITKHKWEQAGEAERLRAYLEGTCVEWLRRY | |
| signal peptide | LKNGNATLLRTDSPKAHVTHHSRPEDKVTLRCWALGFYPADITLTWQLNGEELIQDM | |
| is removed) | ELVETRPAGDGTFQKWASVVVPLGKEQYYTCHVYHQGLPEPLTLRWEPPPSTVSNMA | |
| | TVAVLVVLGAAIVTGAVVAFVMKMRRRNTGGKGGDYALAPGSQTSDLSLPDCKVMVH | |
| | DPHSLA | |
| | GGCCCACACTCGCTGAGGTATTTCGTCACCGCCGTGTCCCGGCCCGGCCTCGGGGAG | 10 |
| | CCCCGGTACATGGAAGTCGGCTACGTGGACGACACGGAGTTCGTGCGCTTCGACAGC | |
| | GACGCGGAGAATCCGAGATATGAGCCGCGGGCGCGGTGGATGGAGCAGGAGGGGCCC | |
| | GAGTATTGGGAGCGGGAGACACAGAAAGCCAAGGGCAATGAGCAGAGTTTCCGAGTG | |
| | GACCTGAGGACCCTGCTCGGCTACTACAACCAGAGCAAGGGCGGCTCTCACACTATT | |
| | CAGGTGATCTCTGGCTGTGAAGTGGGGTCCGACGGGCGACTCCTCCGCGGGTACCAG | |
| | CAGTACGCCTACGACGGCTGCGATTACATCGCCCTGAACGAAGACCTGAAAACGTGG | |
| | ACGGCGGCGGACATGGCGGCGCTGATCACCAAACACAAGTGGGAGCAGGCTGGTGAA | |
| | GCAGAGAGACTCAGGGCCTACCTGGAGGGCACGTGCGTGGAGTGGCTCCGCAGATAC | |
| | CTGAAGAACGGGAACGCGACGCTGCTGCGCACAGATTCCCCAAAGGCCCATGTGACC | |
| | CATCACAGCAGACCTGAAGATAAAGTCACCCTGAGGTGCTGGGCCCTGGGCTTCTAC | |
| | CCTGCTGACATCACCCTGACCTGGCAGTTGAATGGGGAGGAGCTGATCCAGGACATG | |
| | GAGCTTGTGGAGACCAGGCCTGCAGGGGATGGAACCTTCCAGAAGTGGGCATCTGTG | |
| | GTGGTGCCTCTTGGGAAGGAGCAGTATTACACATGCCATGTGTACCATCAGGGGCTG | |
| | CCTGAGCCCCTCACCCTGAGATGGGAGCCTCCTCCATCCACTGTCTCCAACATGGCG | |
| | ACCGTTGCTGTTCTGGTTGTCCTTGGAGCTGCAATAGTCACTGGAGCTGTGGTGGCT | |
| | TTTGTGATGAAGATGAGAAGGAGAAACACAGGTGGAAAAGGAGGGGACTATGCTCTG | |
| | GCTCCAGGCTCCCAGACCTCTGATCTGTCTCTCCCAGATTGTAAAGTGATGGTTCAT | |
| | GACCCTCATTCTCTAGCG | |
| Peptide linker 2 | GGGGGGGGSGGGGSGGGGS | 11 |
| | GGGGGGGGAGGCTCCGGTGGAGGGGGGTCTGGAGGGGGGGGGTCTGGTGGAGGCGGA | 12 |
| | AGT | |
| Single chain | IQKTPQIQVYSRHPPENGKPNILNCYVTQFHPPHIEIQMLKNGKKIPKVEMSDMSFS | 65 |
| MHC class | KDWSFYILAHTEFTPTETDTYACRVKHASMAEPKTVYWDRDMGGGGSGGGGSGGGGS | |
| I molecule | GGGGSGPHSLRYFVTAVSRPGLGEPRYMEVGYVDDTEFVRFDSDAENPRYEPRARWM | |
| (ß₂ | EQEGPEYWERETQKAKGNEQSFRVDLRTLLGYYNQSKGGSHTIQVISGCEVGSDGRL | |
| microglobulin | LRGYQQYAYDGCDYIALNEDLKTWTAADMAALITKHKWEQAGEAERLRAYLEGTCVE | |
| (from which | WLRRYLKNGNATLLRTDSPKAHVTHHSRPEDKVTLRCWALGFYPADITLTWQLNGEE | |
| signal peptide | LIQDMELVETRPAGDGTFQKWASVVVPLGKEQYYTCHVYHQGLPEPLTLRWEPPPST | |
| is removed) + | VSNMATVAVLVVLGAAIVTGAVVAFVMKMRRRNTGGKGGDYALAPGSQTSDLSLPDC | |
| peptide linker | KVMVHDPHSLA | |
| 2 + MHC class | ATCCAGAAAACCCCTCAAATTCAAGTATACTCACGCCACCCACCGGAGAATGGGAAG | 66 |
| Iα chain | CCGAACATACTGAACTGCTACGTAACACAGTTCCACCCGCCTCACATTGAAATCCAA | |
| (from which | ATGCTGAAGAACGGGAAAAAAATTCCTAAAGTAGAGATGTCAGATATGTCCTTCAGC | |
| signal peptide | AAGGACTGGTCTTTCTATATCCTGGCTCACACTGAATTCACCCCCACTGAGACTGAT | |
| is removed)) | ACATACGCCTGCAGAGTTAAGCATGCCAGTATGGCCGAGCCCAAGACCGTCTACTGG | |
| | GATCGAGACATGGGGGGGGGAGGCTCCGGTGGAGGGGGGTCTGGAGGGGGGGGGTCT | |
| | GGTGGAGGCGGAAGTGGCCCACACTCGCTGAGGTATTTCGTCACCGCCGTGTCCCGG | |
| | CCCGGCCTCGGGGAGCCCCGGTACATGGAAGTCGGCTACGTGGACGACACGGAGTTC | |
| | GTGCGCTTCGACAGCGACGCGGAGAATCCGAGATATGAGCCGCGGGCGCGGTGGATG | |
| | GAGCAGGAGGGGCCCGAGTATTGGGAGCGGGAGACACAGAAAGCCAAGGGCAATGAG | |
| | CAGAGTTTCCGAGTGGACCTGAGGACCCTGCTCGGCTACTACAACCAGAGCAAGGGC | |
| | GGCTCTCACACTATTCAGGTGATCTCTGGCTGTGAAGTGGGGTCCGACGGGCGACTC | |
| | CTCCGCGGGTACCAGCAGTACGCCTACGACGGCTGCGATTACATCGCCCTGAACGAA | |
| | GACCTGAAAACGTGGACGGCGGCGGACATGGCGGCGCTGATCACCAAACACAAGTGG | |
| | GAGCAGGCTGGTGAAGCAGAGAGACTCAGGGCCTACCTGGAGGGCACGTGCGTGGAG | |
| | TGGCTCCGCAGATACCTGAAGAACGGGAACGCGACGCTGCTGCGCACAGATTCCCCA | |
| | AAGGCCCATGTGACCCATCACAGCAGACCTGAAGATAAAGTCACCCTGAGGTGCTGG | |
| | GCCCTGGGCTTCTACCCTGCTGACATCACCCTGACCTGGCAGTTGAATGGGGAGGAG | |
| | CTGATCCAGGACATGGAGCTTGTGGAGACCAGGCCTGCAGGGGATGGAACCTTCCAG | |
| | AAGTGGGCATCTGTGGTGGTGCCTCTTGGGAAGGAGCAGTATTACACATGCCATGTG | |
| | TACCATCAGGGGCTGCCTGAGCCCCTCACCCTGAGATGGGAGCCTCCTCCATCCACT | |
| | GTCTCCAACATGGCGACCGTTGCTGTTCTGGTTGTCCTTGGAGCTGCAATAGTCACT | |
| | GGAGCTGTGGTGGCTTTTGTGATGAAGATGAGAAGGAGAAACACAGGTGGAAAAGGA | |
| | GGGGACTATGCTCTGGCTCCAGGCTCCCAGACCTCTGATCTGTCTCTCCCAGATTGT | |
| | AAAGTGATGGTTCATGACCCTCATTCTCTAGCG | |
| sc-Trimer | MARSVTLVFLVLVSLTGLYASIINFEKLGGGASGGGGSGGGGSIQKTPQIQVYSRHP | 13 |
| (OVA peptide 1 + | PENGKPNILNCYVTQFHPPHIEIQMLKNGKKIPKVEMSDMSFSKDWSFYILAHTEFT | |
| peptide linker | PTETDTYACRVKHASMAEPKTVYWDRDMGGGGSGGGGSGGGGSGGGGSGPHSLRYFV | |
| 1 + single chain | TAVSRPGLGEPRYMEVGYVDDTEFVRFDSDAENPRYEPRARWMEQEGPEYWERETQK | |
| MHC class I | AKGNEQSFRVDLRTLLGYYNQSKGGSHTIQVISGCEVGSDGRLLRGYQQYAYDGCDY | |
| molecule) | IALNEDLKTWTAADMAALITKHKWEQAGEAERLRAYLEGTCVEWLRRYLKNGNATLL | |
| | RTDSPKAHVTHHSRPEDKVTLRCWALGFYPADITLTWQLNGEELTQDMELVETRPAG | |
| | DGTFQKWASVVVPLGKEQYYTCHVYHQGLPEPLTLRWEPPPSTVSNMATVAVLVVLG | |
| | AAIVTGAVVAFVMKMRRRNTGGKGGDYALAPGSQTSDLSLPDCKVMVHDPHSLA | |
| | ATGGCTCGCTCGGTGACCCTGGTCTTTCTGGTGCTTGTCTCACTGACCGGCCTGTAT | |
| | GCTTCCATTATAAATTTTGAAAAGTTGGGCGGAGGTGCCTCTGGCGGTGGGGGCAGC | 14 |
| | GGTGGAGGGGGCAGTATCCAGAAAACCCCTCAAATTCAAGTATACTCACGCCACCCA | |
| | CCGGAGAATGGGAAGCCGAACATACTGAACTGCTACGTAACACAGTTCCACCCGCCT | |
| | CACATTGAAATCCAAATGCTGAAGAACGGGAAAAAAATTCCTAAAGTAGAGATGTCA | |
| | GATATGTCCTTCAGCAAGGACTGGTCTTTCTATATCCTGGCTCACACTGAATTCACC | |
| | CCCACTGAGACTGATACATACGCCTGCAGAGTTAAGCATGCCAGTATGGCCGAGCCC | |
| | AAGACCGTCTACTGGGATCGAGACATGGGGGGGGGAGGCTCCGGTGGAGGGGGGTCT | |
| | GGAGGGGGGGGGTCTGGTGGAGGCGGAAGTGGCCCACACTCGCTGAGGTATTTCGTC | |
| | ACCGCCGTGTCCCGGCCCGGCCTCGGGGAGCCCCGGTACATGGAAGTCGGCTACGTG | |
| | GACGACACGGAGTTCGTGCGCTTCGACAGCGACGCGGAGAATCCGAGATATGAGCCG | |
| | CGGGCGCGGTGGATGGAGCAGGAGGGGCCCGAGTATTGGGAGCGGGAGACACAGAAA | |
| | GCCAAGGGCAATGAGCAGAGTTTCCGAGTGGACCTGAGGACCCTGCTCGGCTACTAC | |
| | AACCAGAGCAAGGGCGGCTCTCACACTATTCAGGTGATCTCTGGCTGTGAAGTGGGG | |
| | TCCGACGGGCGACTCCTCCGCGGGTACCAGCAGTACGCCTACGACGGCTGCGATTAC | |
| | ATCGCCCTGAACGAAGACCTGAAAACGTGGACGGCGGCGGACATGGCGGCGCTGATC | |
| | ACCAAACACAAGTGGGAGCAGGCTGGTGAAGCAGAGAGACTCAGGGCCTACCTGGAG | |
| | GGCACGTGCGTGGAGTGGCTCCGCAGATACCTGAAGAACGGGAACGCGACGCTGCTG | |
| | CGCACAGATTCCCCAAAGGCCCATGTGACCCATCACAGCAGACCTGAAGATAAAGTC | |
| | ACCCTGAGGTGCTGGGCCCTGGGCTTCTACCCTGCTGACATCACCCTGACCTGGCAG | |
| | TTGAATGGGGAGGAGCTGATCCAGGACATGGAGCTTGTGGAGACCAGGCCTGCAGGG | |
| | GATGGAACCTTCCAGAAGTGGGCATCTGTGGTGGTGCCTCTTGGGAAGGAGCAGTAT | |
| | TACACATGCCATGTGTACCATCAGGGGCTGCCTGAGCCCCTCACCCTGAGATGGGAG | |
| | CCTCCTCCATCCACTGTCTCCAACATGGCGACCGTTGCTGTTCTGGTTGTCCTTGGA | |
| | GCTGCAATAGTCACTGGAGCTGTGGTGGCTTTTGTGATGAAGATGAGAAGGAGAAAC | |
| | ACAGGTGGAAAAGGAGGGGACTATGCTCTGGCTCCAGGCTCCCAGACCTCTGATCTG | |
| | TCTCTCCCAGATTGTAAAGTGATGGTTCATGACCCTCATTCTCTAGCG | |
| CD81 | MGVEGCTKCIKYLLFVFNFVFWLAGGVILGVALWLRHDPQTTSLLYLELGNKPAPNT | 15 |
| | FYVGIYILIAVGAVMMFVGFLGCYGAIQESQCLLGTFFTCLVILFACEVAAGIWGFV | |
| | NKDQIAKDVKQFYDQALQQAVMDDDANNAKAVVKTFHETLNCCGSNALTTLTTTILR | |
| | NSLCPSGGNILTPLLQQDCHQKIDELFSGKLYLIGIAAIVVAVIMIFEMILSMVLCC | |
| | GIRNSSVY | |
| | ATGGGGGTGGAGGGCTGCACCAAATGCATCAAATACCTGCTCTTCGTCTTCAATTTC | 16 |
| | GTCTTCTGGCTGGCTGGAGGCGTGATCCTAGGTGTAGCTCTGTGGTTGCGTCATGAT | |
| | CCACAGACCACCAGCCTGCTGTACCTGGAACTGGGAAACAAACCGGCACCCAACACC | |
| | TTCTACGTGGGCATCTACATTCTCATTGCTGTGGGAGCTGTGATGATGTTTGTAGGC | |
| | TTCCTGGGGTGCTATGGGGCCATCCAGGAGTCCCAGTGTCTGCTGGGGACGTTCTTC | |
| | ACCTGCCTTGTGATCCTGTTTGCCTGTGAGGTGGCTGCAGGCATCTGGGGCTTCGTA | |
| | AACAAAGACCAGATCGCCAAGGATGTGAAGCAGTTCTATGACCAGGCCCTTCAGCAA | |
| | GCTGTGATGGATGATGATGCCAACAATGCCAAGGCTGTGGTGAAGACTTTCCATGAG | |
| | ACGCTCAACTGTTGTGGCTCCAACGCACTGACCACACTGACTACCACCATACTGAGG | |
| | AACAGCCTGTGTCCCTCAGGCGGCAACATACTCACCCCCTTACTGCAGCAAGATTGT | |
| | CATCAGAAAATCGATGAGCTCTTCTCTGGGAAGCTGTACCTCATTGGAATTGCAGCC | |
| | ATTGTGGTAGCTGTCATTATGATCTTTGAGATGATTCTGAGCATGGTGCTGTGCTGT | |
| | GGCATCCGGAACAGCTCCGTGTACTGA | |
| sc-Trimer- | MARSVTLVFLVLVSLTGLYASIINFEKLGGGASGGGGSGGGGSIQKTPQIQVYSRHP | 17 |
| CD81 | PENGKPNILNCYVTQFHPPHIEIQMLKNGKKIPKVEMSDMSFSKDWSFYILAHTEFT | |
| (sc-Trimer + | PTETDTYACRVKHASMAEPKTVYWDRDMGGGGSGGGGSGGGGSGGGGSGPHSLRYFV | |
| CD81) | TAVSRPGLGEPRYMEVGYVDDTEFVRFDSDAENPRYEPRARWMEQEGPEYWERETQK | |
| | AKGNEQSFRVDLRTLLGYYNQSKGGSHTIQVISGCEVGSDGRLLRGYQQYAYDGCDY | |
| | IALNEDLKTWTAADMAALITKHKWEQAGEAERLRAYLEGTCVEWLRRYLKNGNATLL | |
| | RTDSPKAHVTHHSRPEDKVTLRCWALGFYPADITLTWQLNGEELIQDMELVETRPAG | |
| | DGTFQKWASVVVPLGKEQYYTCHVYHQGLPEPLTLRWEPPPSTVSNMATVAVLVVLG | |
| | AAIVTGAVVAFVMKMRRRNTGGKGGDYALAPGSQTSDLSLPDCKVMVHDPHSLAMGV | |
| | EGCTKCIKYLLFVFNFVFWLAGGVILGVALWLRHDPQTTSLLYLELGNKPAPNTFYV | |
| | GIYILIAVGAVMMFVGFLGCYGAIQESQCLLGTFFTCLVILFACEVAAGIWGFVNKD | |
| | QIAKDVKQFYDQALQQAVMDDDANNAKAVVKTFHETLNCCGSNALTTLTTTILRNSL | |
| | CPSGGNILTPLLQQDCHQKTDELFSGKLYLIGTAATVVAVIMIFEMTLSMVLCCGIR | |
| | NSSVY | |
| | ATGGCTCGCTCGGTGACCCTGGTCTTTCTGGTGCTTGTCTCACTGACCGGCCTGTAT | 18 |
| | GCTTCCATTATAAATTTTGAAAAGTTGGGCGGAGGTGCCTCTGGCGGTGGGGGCAGC | |
| | GGTGGAGGGGGCAGTATCCAGAAAACCCCTCAAATTCAAGTATACTCACGCCACCCA | |
| | CCGGAGAATGGGAAGCCGAACATACTGAACTGCTACGTAACACAGTTCCACCCGCCT | |
| | CACATTGAAATCCAAATGCTGAAGAACGGGAAAAAAATTCCTAAAGTAGAGATGTCA | |
| | GATATGTCCTTCAGCAAGGACTGGTCTTTCTATATCCTGGCTCACACTGAATTCACC | |
| | CCCACTGAGACTGATACATACGCCTGCAGAGTTAAGCATGCCAGTATGGCCGAGCCC | |
| | AAGACCGTCTACTGGGATCGAGACATGGGGGGGGGAGGCTCCGGTGGAGGGGGGTCT | |
| | GGAGGGGGGGGGTCTGGTGGAGGCGGAAGTGGCCCACACTCGCTGAGGTATTTCGTC | |
| | ACCGCCGTGTCCCGGCCCGGCCTCGGGGAGCCCCGGTACATGGAAGTCGGCTACGTG | |
| | GACGACACGGAGTTCGTGCGCTTCGACAGCGACGCGGAGAATCCGAGATATGAGCCG | |
| | CGGGCGCGGTGGATGGAGCAGGAGGGGCCCGAGTATTGGGAGCGGGAGACACAGAAA | |
| | GCCAAGGGCAATGAGCAGAGTTTCCGAGTGGACCTGAGGACCCTGCTCGGCTACTAC | |
| | AACCAGAGCAAGGGCGGCTCTCACACTATTCAGGTGATCTCTGGCTGTGAAGTGGGG | |
| | TCCGACGGGCGACTCCTCCGCGGGTACCAGCAGTACGCCTACGACGGCTGCGATTAC | |
| | ATCGCCCTGAACGAAGACCTGAAAACGTGGACGGCGGCGGACATGGCGGCGCTGATC | |
| | ACCAAACACAAGTGGGAGCAGGCTGGTGAAGCAGAGAGACTCAGGGCCTACCTGGAG | |
| | GGCACGTGCGTGGAGTGGCTCCGCAGATACCTGAAGAACGGGAACGCGACGCTGCTG | |
| | CGCACAGATTCCCCAAAGGCCCATGTGACCCATCACAGCAGACCTGAAGATAAAGTC | |
| | ACCCTGAGGTGCTGGGCCCTGGGCTTCTACCCTGCTGACATCACCCTGACCTGGCAG | |
| | TTGAATGGGGAGGAGCTGATCCAGGACATGGAGCTTGTGGAGACCAGGCCTGCAGGG | |
| | GATGGAACCTTCCAGAAGTGGGCATCTGTGGTGGTGCCTCTTGGGAAGGAGCAGTAT | |
| | TACACATGCCATGTGTACCATCAGGGGCTGCCTGAGCCCCTCACCCTGAGATGGGAG | |
| | CCTCCTCCATCCACTGTCTCCAACATGGCGACCGTTGCTGTTCTGGTTGTCCTTGGA | |
| | GCTGCAATAGTCACTGGAGCTGTGGTGGCTTTTGTGATGAAGATGAGAAGGAGAAAC | |
| | ACAGGTGGAAAAGGAGGGGACTATGCTCTGGCTCCAGGCTCCCAGACCTCTGATCTG | |
| | TCTCTCCCAGATTGTAAAGTGATGGTTCATGACCCTCATTCTCTAGCGATGGGGGTG | |
| | GAGGGCTGCACCAAATGCATCAAATACCTGCTCTTCGTCTTCAATTTCGTCTTCTGG | |
| | CTGGCTGGAGGCGTGATCCTAGGTGTAGCTCTGTGGTTGCGTCATGATCCACAGACC | |
| | ACCAGCCTGCTGTACCTGGAACTGGGAAACAAACCGGCACCCAACACCTTCTACGTG | |
| | GGCATCTACATTCTCATTGCTGTGGGAGCTGTGATGATGTTTGTAGGCTTCCTGGGG | |
| | TGCTATGGGGCCATCCAGGAGTCCCAGTGTCTGCTGGGGACGTTCTTCACCTGCCTT | |
| | GTGATCCTGTTTGCCTGTGAGGTGGCTGCAGGCATCTGGGGCTTCGTAAACAAAGAC | |
| | CAGATCGCCAAGGATGTGAAGCAGTTCTATGACCAGGCCCTTCAGCAAGCTGTGATG | |
| | GATGATGATGCCAACAATGCCAAGGCTGTGGTGAAGACTTTCCATGAGACGCTCAAC | |
| | TGTTGTGGCTCCAACGCACTGACCACACTGACTACCACCATACTGAGGAACAGCCTG | |
| | TGTCCCTCAGGCGGCAACATACTCACCCCCTTACTGCAGCAAGATTGTCATCAGAAA | |
| | ATCGATGAGCTCTTCTCTGGGAAGCTGTACCTCATTGGAATTGCAGCCATTGTGGTA | |
| | GCTGTCATTATGATCTTTGAGATGATTCTGAGCATGGTGCTGTGCTGTGGCATCCGG | |
| | AACAGCTCCGTGTACTGA | |

TABLE 2

| | | SEQ |
|---|---|---|
| | Sequence | ID NO: |
| CD80 | MACNCQLMQDTPLLKFPCPRLILLFVLLIRLSQVSSDVDEQLSKSVKDKVLLPCRYN | 19 |
| | SPHEDESEDRIYWQKHDKVVLSVIAGKLKVWPEYKNRTLYDNTTYSLIILGLVLSDR | |
| | GTYSCVVQKKERGTYEVKHLALVKLSIKADFSTPNITESGNPSADTKRITCFASGGF | |
| | PKPRFSWLENGRELPGINTTISQDPESELYTISSQLDFNTTRNHTIKCLIKYGDAHV | |
| | SEDFTWEKPPEDPPDSKNTLVLFGAGFGAVITVVVIVVIIKCFCKHRSCFRRNEASR | |
| | ETNNSLTFGPEEALAEQTVFL | |
| | ATGGCTTGCAATTGTCAGTTGATGCAGGATACACCACTCCTCAAGTTTCCATGTCCA | 20 |
| | AGGCTCATTCTTCTCTTTGTGCTGCTGATTCGTCTTTCACAAGTGTCTTCAGATGTT | |
| | GATGAACAACTGTCCAAGTCAGTGAAAGATAAGGTATTGCTGCCTTGCCGTTACAAC | |
| | TCTCCTCATGAAGATGAGTCTGAAGACCGAATCTACTGGCAAAAACATGACAAAGTG | |
| | GTGCTGTCTGTCATTGCTGGGAAACTAAAAGTGTGGCCCGAGTATAAGAACCGGACT | |
| | TTATATGACAACACTACCTACTCTCTTATCATCCTGGGCCTGGTCCTTTCAGACCGG | |
| | GGCACATACAGCTGTGTCGTTCAAAAGAAGGAAAGAGGAACGTATGAAGTTAAACAC | |
| | TTGGCTTTAGTAAAGTTGTCCATCAAAGCTGACTTCTCTACCCCCAACATAACTGAG | |
| | TCTGGAAACCCATCTGCAGACACTAAAAGGATTACCTGCTTTGCTTCCGGGGGTTTC | |
| | CCAAAGCCTCGCTTCTCTTGGTTGGAAAATGGAAGAGAATTACCTGGCATCAATACG | |
| | ACAATTTCCCAGGATCCTGAATCTGAATTGTACACCATTAGTAGCCAACTAGATTTC | |
| | AATACGACTCGCAACCACACCATTAAGTGTCTCATTAAATATGGAGATGCTCACGTG | |
| | TCAGAGGACTTCACCTGGGAAAAACCCCCAGAAGACCCTCCTGATAGCAAGAACACA | |
| | CTTGTGCTCTTTGGGGCAGGATTCGGCGCAGTAATAACAGTCGTCGTCATCGTTGTC | |
| | ATCATCAAATGCTTCTGTAAGCACAGAAGCTGTTTCAGAAGAAATGAGGCAAGCAGA | |
| | GAAACAAACAACAGCCTTACCTTCGGGCCTGAAGAAGCATTAGCTGAACAGACCGTC | |
| | TTCCTTTAG | |
| CD9 | MPVKGGSKCIKYLLFGFNFIFWLAGIAVLAIGLWLRFDSQTKSIFEQENNHSSFYTG | 21 |
| | VYILIGAGALMMLVGFLGCCGAVQESQCMLGLFFGFLLVIFAIEIAAAVWGYTHKDE | |
| | VIKELQEFYKDTYQKLRSKDEPQRETLKAIHMALDCCGIAGPLEQFISDTCPKKQLL | |
| | ESFQVKPCPEAISEVENNKFHIIGAVGIGIAVVMIFGMIFSMILCCAIRRSREMV | |
| | ATGCCGGTCAAAGGAGGTAGCAAGTGCATCAAATACCTGCTCTTCGGATTTAACTTC | 22 |
| | ATCTTCTGGCTCGCTGGCATTGCAGTGCTTGCTATTGGACTATGGCTCCGATTCGAC | |
| | TCTCAGACCAAGAGCATCTTCGAGCAAGAGAATAACCATTCCAGTTTCTACACAGGA | |
| | GTGTACATTCTGATTGGAGCCGGGGCCCTCATGATGCTGGTTGGTTTCCTGGGCTGC | |
| | TGTGGAGCTGTACAAGAGTCCCAGTGCATGCTGGGATTGTTCTTCGGGTTCCTCTTG | |
| | GTGATATTCGCCATTGAGATAGCCGCCGCCGTCTGGGGCTATACCCACAAGGATGAG | |
| | GTGATTAAAGAACTCCAGGAGTTTTACAAGGACACCTACCAAAAGTTACGGAGCAAG | |
| | GATGAACCCCAGCGGGAAACACTCAAAGCCATCCATATGGCGTTGGACTGCTGTGGC | |
| | ATAGCTGGTCCTTTGGAGCAGTTTATCTCGGACACCTGCCCCAAGAAACAGCTTTTG | |
| | GAAAGTTTCCAGGTTAAGCCCTGCCCTGAAGCCATCAGTGAGGTCTTCAACAACAAG | |
| | TTCCACATCATTGGAGCAGTGGGTATCGGCATCGCCGTGGTGATGATCTTCGGCATG | |
| | ATCTTCAGCATGATCCTGTGCTGCGCCATCCGCAGGAGCCGAGAAATGGTCTAG | |
| CD80- | MACNCQLMQDTPLLKFPCPRLILLFVLLIRLSQVSSDVDEQLSKSVKDKVLLPCRYN | 23 |
| CD9 | SPHEDESEDRIYWQKHDKVVLSVIAGKLKVWPEYKNRTLYDNTTYSLIILGLVLSDR | |
| | GTYSCVVQKKERGTYEVKHLALVKLSIKADFSTPNITESGNPSADTKRITCFASGGF | |
| | PKPRFSWLENGRELPGINTTISQDPESELYTISSQLDFNTTRNHTIKCLIKYGDAHV | |
| | SEDFTWEKPPEDPPDSKNTLVLFGAGFGAVITVVVIVVIIKCFCKHRSCFRRNEASR | |
| | ETNNSLTFGPEEALAEQTVFLMPVKGGSKCIKYLLFGFNFIFWLAGIAVLAIGLWLR | |
| | FDSQTKSIFEQENNHSSFYTGVYILIGAGALMMLVGFLGCCGAVQESQCMLGLFFGF | |
| | LLVIFAIEIAAAVWGYTHKDEVIKELQEFYKDTYQKLRSKDEPQRETLKAIHMALDC | |
| | CGIAGPLEQFISDTCPKKQLLESFQVKPCPEAISEVFNNKFHIIGAVGIGIAVVMIF | |
| | GMIFSMILCCAIRRSREMV | |
| | ATGGCTTGCAATTGTCAGTTGATGCAGGATACACCACTCCTCAAGTTTCCATGTCCA | 24 |
| | AGGCTCATTCTTCTCTTTGTGCTGCTGATTCGTCTTTCACAAGTGTCTTCAGATGTT | |
| | GATGAACAACTGTCCAAGTCAGTGAAAGATAAGGTATTGCTGCCTTGCCGTTACAAC | |
| | TCTCCTCATGAAGATGAGTCTGAAGACCGAATCTACTGGCAAAAACATGACAAAGTG | |
| | GTGCTGTCTGTCATTGCTGGGAAACTAAAAGTGTGGCCCGAGTATAAGAACCGGACT | |
| | TTATATGACAACACTACCTACTCTCTTATCATCCTGGGCCTGGTCCTTTCAGACCGG | |
| | GGCACATACAGCTGTGTCGTTCAAAAGAAGGAAAGAGGAACGTATGAAGTTAAACAC | |
| | TTGGCTTTAGTAAAGTTGTCCATCAAAGCTGACTTCTCTACCCCCAACATAACTGAG | |
| | TCTGGAAACCCATCTGCAGACACTAAAAGGATTACCTGCTTTGCTTCCGGGGGTTTC | |
| | CCAAAGCCTCGCTTCTCTTGGTTGGAAAATGGAAGAGAATTACCTGGCATCAATACG | |
| | ACAATTTCCCAGGATCCTGAATCTGAATTGTACACCATTAGTAGCCAACTAGATTTC | |
| | AATACGACTCGCAACCACACCATTAAGTGTCTCATTAAATATGGAGATGCTCACGTG | |
| | TCAGAGGACTTCACCTGGGAAAAACCCCCAGAAGACCCTCCTGATAGCAAGAACACA | |
| | CTTGTGCTCTTTGGGGCAGGATTCGGCGCAGTAATAACAGTCGTCGTCATCGTTGTC | |
| | ATCATCAAATGCTTCTGTAAGCACAGAAGCTGTTTCAGAAGAAATGAGGCAAGCAGA | |
| | GAAACAAACAACAGCCTTACCTTCGGGCCTGAAGAAGCATTAGCTGAACAGACCGTC | |
| | TTCCTTATGCCGGTCAAAGGAGGTAGCAAGTGCATCAAATACCTGCTCTTCGGATTT | |
| | AACTTCATCTTCTGGCTCGCTGGCATTGCAGTGCTTGCTATTGGACTATGGCTCCGA | |
| | TTCGACTCTCAGACCAAGAGCATCTTCGAGCAAGAGAATAACCATTCCAGTTTCTAC | |
| | ACAGGAGTGTACATTCTGATTGGAGCCGGGGCCCTCATGATGCTGGTTGGTTTCCTG | |
| | GGCTGCTGTGGAGCTGTACAAGAGTCCCAGTGCATGCTGGGATTGTTCTTCGGGTTC | |
| | CTCTTGGTGATATTCGCCATTGAGATAGCCGCCGCCGTCTGGGGCTATACCCACAAG | |
| | GATGAGGTGATTAAAGAACTCCAGGAGTTTTACAAGGACACCTACCAAAAGTTACGG | |
| | AGCAAGGATGAACCCCAGCGGGAAACACTCAAAGCCATCCATATGGCGTTGGACTGC | |
| | TGTGGCATAGCTGGTCCTTTGGAGCAGTTTATCTCGGACACCTGCCCCAAGAAACAG | |
| | CTTTTGGAAAGTTTCCAGGTTAAGCCCTGCCCTGAAGCCATCAGTGAGGTCTTCAAC | |
| | AACAAGTTCCACATCATTGGAGCAGTGGGTATCGGCATCGCCGTGGTGATGATCTTC | |
| | GGCATGATCTTCAGCATGATCCTGTGCTGCGCCATCCGCAGGAGCCGAGAAATGGTC | |
| | TAG | |

TABLE 3

| | | SEQ |
|---|---|---|
| | Sequence | ID NO: |
| IL-2 | APTSSSTSSSTAEAQQQQQQQQQQQQHLEQLLMDLQELLSRMENYRNLKLPRMLTFK | 25 |
| (from which | FYLPKQATELKDLQCLEDELGPLRHVLDLTQSKSFQLEDAENFISNIRVTVVKLKGS | |
| signal peptide | DNTFECQFDDESATVVDFLRRWIAFCQSIISTSPQ | |
| is removed) | GCACCCACTTCAAGCTCCACTTCAAGCTCTACAGCGGAAGCACAGCAGCAGCAGCAG | 26 |
| | CAGCAGCAGCAGCAGCAGCAGCACCTGGAGCAGCTGTTGATGGACCTACAGGAGCTC | |
| | CTGAGCAGGATGGAGAATTACAGGAACCTGAAACTCCCCAGGATGCTCACCTTCAAA | |
| | TTTTACTTGCCCAAGCAGGCCACAGAATTGAAAGATCTTCAGTGCCTAGAAGATGAA | |
| | CTTGGACCTCTGCGGCATGTTCTGGATTTGACTCAAAGCAAAAGCTTTCAATTGGAA | |
| | GATGCTGAGAATTTCATCAGCAATATCAGAGTAACTGTTGTAAAACTAAAGGGCTCT | |
| | GACAACACATTTGAGTGCCAATTCGATGATGAGTCAGCAACTGTGGTGGACTTTCTG | |
| | AGGAGATGGATAGCCTTCTGTCAAAGCATCATCTCAACAAGCCCTCAA | |
| CD63 | MAVEGGMKCVKFLLYVLLLAFCACAVGLIAIGVAVQVVLKQAITHETTAGSLLPVVI | 27 |
| | IAVGAFLFLVAFVGCCGACKENYCLMITFAIFLSLIMLVEVAVAIAGYVFRDQVKSE | |
| | FNKSFQQQMQNYLKDNKTATILDKLQKENNCCGASNYTDWENIPGMAKDRVPDSCCI | |
| | NITVGCGNDFKESTIHTQGCVETIAIWLRKNILLVAAAALGIAFVEVLGIIFSCCLV | |
| | KSIRSGYEVM | |
| | ATGGCGGTGGAAGGAGGAATGAAGTGTGTCAAGTTTTTGCTCTACGTTCTCCTGCTG | 28 |
| | GCCTTCTGCGCCTGTGCAGTGGGATTGATCGCCATTGGTGTAGCGGTTCAGGTTGTC | |
| | TTGAAGCAGGCCATTACCCATGAGACTACTGCTGGCTCGCTGTTGCCTGTGGTCATC | |
| | ATTGCAGTGGGTGCCTTCCTCTTCCTGGTGGCCTTTGTGGGCTGCTGTGGGGCCTGC | |
| | AAGGAGAACTACTGTCTCATGATTACATTTGCCATCTTCCTGTCTCTTATCATGCTT | |
| | GTGGAGGTGGCTGTGGCCATTGCTGGCTATGTGTTTAGAGACCAGGTGAAGTCAGAG | |
| | TTTAATAAAAGCTTCCAGCAGCAGATGCAGAATTACCTTAAAGACAACAAAACAGCC | |
| | ACTATTTTGGACAAATTGCAGAAAGAAAATAACTGCTGTGGAGCTTCTAACTACACA | |
| | GACTGGGAAAACATCCCCGGCATGGCCAAGGACAGAGTCCCCGATTCTTGCTGCATC | |
| | AACATAACTGTGGGCTGTGGGAATGATTTCAAGGAATCCACTATCCATACCCAGGGC | |
| | TGCGTGGAGACTATAGCAATATGGCTAAGGAAGAACATACTGCTGGTGGCTGCAGCG | |
| | GCCCTGGGCATTGCTTTTGTGGAGGTCTTGGGAATTATCTTCTCCTGCTGTCTGGTG | |
| | AAGAGTATTCGAAGTGGCTATGAAGTAATGTAG | |
| CD63 | MAVEGGMKCVKFLLYVLLLAFCACAVGLIAIGVAVQVVLKQAITHETTAGSLLPVVI | 57 |
| (amino acids | IAVGAFLFLVAFVGCCGACKENYCLMITFAIFLSLIMLVEVAVAIAGYVFRDQVKSE | |
| 1 to 170) | FNKSFQQQMQNYLKDNKTATILDKLQKENNCCGASNYTDWENIPGMAKDRVPDSCC | |
| | ATGGCGGTGGAAGGAGGAATGAAGTGTGTCAAGTTTTTGCTCTACGTTCTCCTGCTG | 58 |
| | GCCTTCTGCGCCTGTGCAGTGGGATTGATCGCCATTGGTGTAGCGGTTCAGGTTGTC | |
| | TTGAAGCAGGCCATTACCCATGAGACTACTGCTGGCTCGCTGTTGCCTGTGGTCATC | |
| | ATTGCAGTGGGTGCCTTCCTCTTCCTGGTGGCCTTTGTGGGCTGCTGTGGGGCCTGC | |
| | AAGGAGAACTACTGTCTCATGATTACATTTGCCATCTTCCTGTCTCTTATCATGCTT | |
| | GTGGAGGTGGCTGTGGCCATTGCTGGCTATGTGTTTAGAGACCAGGTGAAGTCAGAG | |
| | TTTAATAAAAGCTTCCAGCAGCAGATGCAGAATTACCTTAAAGACAACAAAACAGCC | |
| | ACTATTTTGGACAAATTGCAGAAAGAAAATAACTGCTGTGGAGCTTCTAACTACACA | |
| | GACTGGGAAAACATCCCCGGCATGGCCAAGGACAGAGTCCCCGATTCTTGCTGC | |
| CD63 | INITVGCGNDFKESTIHTQGCVETIAIWLRKNILLVAAAALGIAFVEVLGIIFSCCL | 59 |
| (amino acids | VKSIRSGYEVM | |
| 171 to 238) | ATCAACATAACTGTGGGCTGTGGGAATGATTTCAAGGAATCCACTATCCATACCCAG | 60 |
| | GGCTGCGTGGAGACTATAGCAATATGGCTAAGGAAGAACATACTGCTGGTGGCTGCA | |
| | GCGGCCCTGGGCATTGCTTTTGTGGAGGTCTTGGGAATTATCTTCTCCTGCTGTCTG | |
| | GTGAAGAGTATTCGAAGTGGCTATGAAGTAATGTAG | |
| Peptide linker 3 | GGGGS | 29 |
| | GGAGGAGGAGGAAGC | 30 |
| CD63-IL2 | MAVEGGMKCVKFLLYVLLLAFCACAVGLIAIGVAVQVVLKQAITHETTAGSLLPVVI | 31 |
| | IAVGAFLFLVAFVGCCGACKENYCLMITFAIFLSLIMLVEVAVAIAGYVFRDQVKSE | |
| | FNKSFQQQMQNYLKDNKTATILDKLQKENNCCGASNYTDWENIPGMAKDRVPDSCCG | |
| | GGGSAPTSSSTSSSTAEAQQQQQQQQQQQQHLEQLLMDLQELLSRMENYRNLKLPRM | |
| | LTFKFYLPKQATELKDLQCLEDELGPLRHVLDLTQSKSFQLEDAENFISNIRVTVVK | |
| | LKGSDNTFECQFDDESATVVDFLRRWTAFCQSTTSTSPQGGGGSINTTVGCGNDFKE | |
| | STIHTQGCVETIAIWLRKNILLVAAAALGIAFVEVLGIIFSCCLVKSIRSGYEVM | |
| | ATGGCGGTGGAAGGAGGAATGAAGTGTGTCAAGTTTTTGCTCTACGTTCTCCTGCTG | 32 |
| | GCCTTCTGCGCCTGTGCAGTGGGATTGATCGCCATTGGTGTAGCGGTTCAGGTTGTC | |
| | TTGAAGCAGGCCATTACCCATGAGACTACTGCTGGCTCGCTGTTGCCTGTGGTCATC | |
| | ATTGCAGTGGGTGCCTTCCTCTTCCTGGTGGCCTTTGTGGGCTGCTGTGGGGCCTGC | |
| | AAGGAGAACTACTGTCTCATGATTACATTTGCCATCTTCCTGTCTCTTATCATGCTT | |
| | GTGGAGGTGGCTGTGGCCATTGCTGGCTATGTGTTTAGAGACCAGGTGAAGTCAGAG | |
| | TTTAATAAAAGCTTCCAGCAGCAGATGCAGAATTACCTTAAAGACAACAAAACAGCC | |
| | ACTATTTTGGACAAATTGCAGAAAGAAAATAACTGCTGTGGAGCTTCTAACTACACA | |
| | GACTGGGAAAACATCCCCGGCATGGCCAAGGACAGAGTCCCCGATTCTTGCTGCGGA | |
| | GGAGGAGGAAGCGCACCCACTTCAAGCTCCACTTCAAGCTCTACAGCGGAAGCACAG | |
| | CAGCAGCAGCAGCAGCAGCAGCAGCAGCAGCAGCACCTGGAGCAGCTGTTGATGGAC | |
| | CTACAGGAGCTCCTGAGCAGGATGGAGAATTACAGGAACCTGAAACTCCCCAGGATG | |
| | CTCACCTTCAAATTTTACTTGCCCAAGCAGGCCACAGAATTGAAAGATCTTCAGTGC | |
| | CTAGAAGATGAACTTGGACCTCTGCGGCATGTTCTGGATTTGACTCAAAGCAAAAGC | |
| | TTTCAATTGGAAGATGCTGAGAATTTCATCAGCAATATCAGAGTAACTGTTGTAAAA | |
| | CTAAAGGGCTCTGACAACACATTTGAGTGCCAATTCGATGATGAGTCAGCAACTGTG | |
| | GTGGACTTTCTGAGGAGATGGATAGCCTTCTGTCAAAGCATCATCTCAACAAGCCCT | |
| | CAAGGAGGAGGAGGAAGCATCAACATAACTGTGGGCTGTGGGAATGATTTCAAGGAA | |
| | TCCACTATCCATACCCAGGGCTGCGTGGAGACTATAGCAATATGGCTAAGGAAGAAC | |
| | ATACTGCTGGTGGCTGCAGCGGCCCTGGGCATTGCTTTTGTGGAGGTCTTGGGAATT | |
| | ATCTTCTCCTGCTGTCTGGTGAAGAGTATTCGAAGTGGCTATGAAGTAATGTAG | |

TABLE 4

| | | SEQ |
|---|---|---|
| | Sequence | ID NO: |
| Signal peptide | MALQIPSLLLSAAVVVLMVLSSPGTEG | 33 |
| of MHC class | ATGGCTCTGCAGATCCCCAGCCTCCTCCTCTCGGCTGCTGTGGTGGTGCTGATGGTG | 34 |
| IIß chain | CTGAGCAGCCCAGGGACTGAGGGC | |
| OVA | ISQAVHAAHAEINEAGR | 35 |
| peptide 2 | ATATCTCAAGCTGTCCATGCAGCACATGCAGAAATCAATGAAGCAGGCAGA | 36 |
| (for MHC class | | |
| II molecule) | | |
| MHC class | GDSERHFVYQFMGECYFTNGTQRIRYVTRYIYNREEYVRYDSDVGEHRAVTELGRPD | 37 |
| IIß chain | AEYWNSQPEILERTRAELDTVCRHNYEGPETHTSLRRLEQPNVVISLSRTEALNHHN | |
| (from which | TLVCSVTDFYPAKIKVRWFRNGQEETVGVSSTQLIRNGDWTFQVLVMLEMTPRRGEV | |
| signal peptide | YTCHVEHPSLKSPITVEWRAQSESAWSKMLSGIGGCVLGVIFLGLGLFIRHRSQKGP | |
| is removed) | RGPPPAGLLQ | |
| | GGAGACTCCGAAAGGCATTTCGTGTACCAGTTCATGGGCGAGTGCTACTTCACCAAC | 38 |
| | GGGACGCAGCGCATACGATATGTGACCAGATACATCTACAACCGGGAGGAGTACGTG | |
| | CGCTACGACAGCGACGTGGGCGAGCACCGCGCGGTGACCGAGCTGGGGCGGCCAGAC | |
| | GCCGAGTACTGGAACAGCCAGCCGGAGATCCTGGAGCGAACGCGGGCCGAGCTGGAC | |
| | ACGGTGTGCAGACACAACTACGAGGGGCCGGAGACCCACACCTCCCTGCGGCGGCTT | |
| | GAACAGCCCAATGTCGTCATCTCCCTGTCCAGGACAGAGGCCCTCAACCACCACAAC | |
| | ACTCTGGTCTGCTCAGTGACAGATTTCTACCCAGCCAAGATCAAAGTGCGCTGGTTC | |
| | CGGAATGGCCAGGAGGAGACGGTGGGGGTCTCATCCACACAGCTTATTAGGAATGGG | |
| | GACTGGACCTTCCAGGTCCTGGTCATGCTGGAGATGACCCCTCGGCGGGGAGAGGTC | |
| | TACACCTGTCACGTGGAGCATCCCAGCCTGAAGAGCCCCATCACTGTGGAGTGGAGG | |
| | GCACAGTCTGAGTCTGCCTGGAGCAAGATGTTGAGCGGCATCGGGGGCTGCGTGCTT | |
| | GGGGTGATCTTCCTCGGGCTTGGCCTTTTCATCCGTCACAGGAGTCAGAAAGGACCT | |
| | CGAGGCCCTCCTCCAGCAGGGCTCCTGCAG | |
| Peptide linker 4 | GGGGSGGGGSG | 39 |
| | GGAGGAGGAGGAAGCGGAGGAGGAGGAAGCGGA | 10 |
| sc-Dimer | MALQIPSLLLSAAVVVLMVLSSPGTEGISQAVHAAHAEINEAGRGGGGSGGGGSGGD | 41 |
| (OVA peptide 2 + | SERHFVYQFMGECYFTNGTQRIRYVTRYIYNREEYVRYDSDVGEHRAVTELGRPDAE | |
| peptide linker | YWNSQPETLERTRAELDTVCRHNYEGPETHTSLRRLEQPNVVISLSRTEALNHHNTL | |
| 4 + MHC class | VCSVTDFYPAKIKVRWFRNGQEETVGVSSTQLIRNGDWTFQVLVMLEMTPRRGEVYT | |
| IIß chain | CHVEHPSLKSPITVEWRAQSESAWSKMLSGIGGCVLGVIFLGLGLFIRHRSQKGPRG | |
| (from which | PPPAGLLQ | |
| signal peptide | ATGGCTCTGCAGATCCCCAGCCTCCTCCTCTCGGCTGCTGTGGTGGTGCTGATGGTG | 42 |
| is removed)) | CTGAGCAGCCCAGGGACTGAGGGCATATCTCAAGCTGTCCATGCAGCACATGCAGAA | |
| | ATCAATGAAGCAGGCAGAGGAGGAGGAGGAAGCGGAGGAGGAGGAAGCGGAGGAGAC | |
| | TCCGAAAGGCATTTCGTGTACCAGTTCATGGGCGAGTGCTACTTCACCAACGGGACG | |
| | CAGCGCATACGATATGTGACCAGATACATCTACAACCGGGAGGAGTACGTGCGCTAC | |
| | GACAGCGACGTGGGCGAGCACCGCGCGGTGACCGAGCTGGGGCGGCCAGACGCCGAG | |
| | TACTGGAACAGCCAGCCGGAGATCCTGGAGCGAACGCGGGCCGAGCTGGACACGGTG | |
| | TGCAGACACAACTACGAGGGGCCGGAGACCCACACCTCCCTGCGGCGGCTTGAACAG | |
| | CCCAATGTCGTCATCTCCCTGTCCAGGACAGAGGCCCTCAACCACCACAACACTCTG | |
| | GTCTGCTCAGTGACAGATTTCTACCCAGCCAAGATCAAAGTGCGCTGGTTCCGGAAT | |
| | GGCCAGGAGGAGACGGTGGGGGTCTCATCCACACAGCTTATTAGGAATGGGGACTGG | |
| | ACCTTCCAGGTCCTGGTCATGCTGGAGATGACCCCTCGGCGGGGAGAGGTCTACACC | |
| | TGTCACGTGGAGCATCCCAGCCTGAAGAGCCCCATCACTGTGGAGTGGAGGGCACAG | |
| | TCTGAGTCTGCCTGGAGCAAGATGTTGAGCGGCATCGGGGGCTGCGTGCTTGGGGTG | |
| | ATCTTCCTCGGGCTTGGCCTTTTCATCCGTCACAGGAGTCAGAAAGGACCTCGAGGC | |
| | CCTCCTCCAGCAGGGCTCCTGCAG | |
| sc-Dimer- | MALQIPSLLLSAAVVVLMVLSSPGTEGISQAVHAAHAEINEAGRGGGGSGGGGSGGD | 43 |
| CD81 | SERHFVYQFMGECYFTNGTQRIRYVTRYIYNREEYVRYDSDVGEHRAVTELGRPDAE | |
| | YWNSQPEILERTRAELDTVCRHNYEGPETHTSLRRLEQPNVVISLSRTEALNHHNTL | |
| | VCSVTDFYPAKIKVRWFRNGQEETVGVSSTQLIRNGDWTFQVLVMLEMTPRRGEVYT | |
| | CHVEHPSLKSPITVEWRAQSESAWSKMLSGIGGCVLGVIFLGLGLFIRHRSQKGPRG | |
| | PPPAGLLQMGVEGCTKCIKYLLFVFNFVFWLAGGVILGVALWLRHDPQTTSLLYLEL | |
| | GNKPAPNTFYVGIYILIAVGAVMMFVGFLGCYGAIQESQCLLGTFFTCLVILFACEV | |
| | AAGIWGFVNKDQIAKDVKQFYDQALQQAVMDDDANNAKAVVKTFHETLNCCGSNALT | |
| | TLTTTILRNSLCPSGGNILTPLLQQDCHQKIDELFSGKLYLIGTAATVVAVIMIFEM | |
| | ILSMVLCCGIRNSSVY | |
| | ATGGCTCTGCAGATCCCCAGCCTCCTCCTCTCGGCTGCTGTGGTGGTGCTGATGGTG | 44 |
| | CTGAGCAGCCCAGGGACTGAGGGCATATCTCAAGCTGTCCATGCAGCACATGCAGAA | |
| | ATCAATGAAGCAGGCAGAGGAGGAGGAGGAAGCGGAGGAGGAGGAAGCGGAGGAGAC | |
| | TCCGAAAGGCATTTCGTGTACCAGTTCATGGGCGAGTGCTACTTCACCAACGGGACG | |
| | CAGCGCATACGATATGTGACCAGATACATCTACAACCGGGAGGAGTACGTGCGCTAC | |
| | GACAGCGACGTGGGCGAGCACCGCGCGGTGACCGAGCTGGGGCGGCCAGACGCCGAG | |
| | TACTGGAACAGCCAGCCGGAGATCCTGGAGCGAACGCGGGCCGAGCTGGACACGGTG | |
| | TGCAGACACAACTACGAGGGGCCGGAGACCCACACCTCCCTGCGGCGGCTTGAACAG | |
| | CCCAATGTCGTCATCTCCCTGTCCAGGACAGAGGCCCTCAACCACCACAACACTCTG | |
| | GTCTGCTCAGTGACAGATTTCTACCCAGCCAAGATCAAAGTGCGCTGGTTCCGGAAT | |
| | GGCCAGGAGGAGACGGTGGGGGTCTCATCCACACAGCTTATTAGGAATGGGGACTGG | |
| | ACCTTCCAGGTCCTGGTCATGCTGGAGATGACCCCTCGGCGGGGAGAGGTCTACACC | |
| | TGTCACGTGGAGCATCCCAGCCTGAAGAGCCCCATCACTGTGGAGTGGAGGGCACAG | |
| | TCTGAGTCTGCCTGGAGCAAGATGTTGAGCGGCATCGGGGGCTGCGTGCTTGGGGTG | |
| | ATCTTCCTCGGGCTTGGCCTTTTCATCCGTCACAGGAGTCAGAAAGGACCTCGAGGC | |
| | CCTCCTCCAGCAGGGCTCCTGCAGATGGGGGTGGAGGGCTGCACCAAATGCATCAAA | |
| | TACCTGCTCTTCGTCTTCAATTTCGTCTTCTGGCTGGCTGGAGGCGTGATCCTAGGT | |
| | GTAGCTCTGTGGTTGCGTCATGATCCACAGACCACCAGCCTGCTGTACCTGGAACTG | |
| | GGAAACAAACCGGCACCCAACACCTTCTACGTGGGCATCTACATTCTCATTGCTGTG | |
| | GGAGCTGTGATGATGTTTGTAGGCTTCCTGGGGTGCTATGGGGCCATCCAGGAGTCC | |
| | CAGTGTCTGCTGGGGACGTTCTTCACCTGCCTTGTGATCCTGTTTGCCTGTGAGGTG | |
| | GCTGCAGGCATCTGGGGCTTCGTAAACAAAGACCAGATCGCCAAGGATGTGAAGCAG | |
| | TTCTATGACCAGGCCCTTCAGCAAGCTGTGATGGATGATGATGCCAACAATGCCAAG | |
| | GCTGTGGTGAAGACTTTCCATGAGACGCTCAACTGTTGTGGCTCCAACGCACTGACC | |
| | ACACTGACTACCACCATACTGAGGAACAGCCTGTGTCCCTCAGGCGGCAACATACTC | |
| | ACCCCCTTACTGCAGCAAGATTGTCATCAGAAAATCGATGAGCTCTTCTCTGGGAAG | |
| | CTGTACCTCATTGGAATTGCAGCCATTGTGGTAGCTGTCATTATGATCTTTGAGATG | |
| | ATTCTGAGCATGGTGCTGTGCTGTGGCATCCGGAACAGCTCCGTGTACTGA | |
| MHC class | MPRSRALILGVLALTTMLSLCGGEDDIEADHVGTYGISVYQSPGDIGQYTFEFDGDE | 45 |
| IIα chain | LFYVDLDKKETVWMLPEFGQLASFDPQGGLQNIAVVKHNLGVLTKRSNSTPATNEAP | |
| | QATVFPKSPVLLGQPNTLICFVDNIFPPVINITWLRNSKSVADGVYETSFFVNRDYS | |
| | FHKLSYLTFIPSDDDIYDCKVEHWGLEEPVLKHWEPEIPAPMSELTETVVCALGLSV | |
| | GLVGTVVGTIFIIQGLRSGGTSRHPGPL | |
| | ATGCCGCGCAGCAGAGCTCTGATTCTGGGGGTCCTCGCCCTGACCACCATGCTCAGC | |
| | CTCTGTGGAGGTGAAGACGACATTGAGGCCGACCACGTAGGCACCTATGGTATAAGT | 46 |
| | GTATATCAGTCTCCTGGAGACATTGGCCAGTACACATTTGAATTTGATGGTGATGAG | |
| | TTGTTCTATGTGGACTTGGATAAGAAGGAGACTGTCTGGATGCTTCCTGAGTTTGGC | |
| | CAATTGGCAAGCTTTGACCCCCAAGGTGGACTGCAAAACATAGCTGTAGTAAAACAC | |
| | AACTTGGGAGTCTTGACTAAGAGGTCAAATTCCACCCCAGCTACCAATGAGGCTCCT | |
| | CAAGCGACTGTGTTCCCCAAGTCCCCTGTGCTGCTGGGTCAGCCCAACACCCTCATC | |
| | TGCTTTGTGGACAACATCTTCCCTCCTGTGATCAACATCACATGGCTCAGAAATAGC | |
| | AAGTCAGTCGCAGACGGTGTTTATGAGACCAGCTTCTTCGTCAACCGTGACTATTCC | |
| | TTCCACAAGCTGTCTTATCTCACCTTCATCCCTTCTGACGATGACATTTATGACTGC | |
| | AAGGTGGAACACTGGGGCCTGGAGGAGCCGGTTCTGAAACACTGGGAACCTGAGATT | |
| | CCAGCCCCCATGTCAGAGCTGACAGAGACTGTGGTCTGTGCCCTGGGGTTGTCTGTG | |
| | GGCCTTGTGGGCATCGTGGTGGGCACCATCTTCATCATTCAAGGCCTGCGATCAGGT | |
| | GGCACCTCCAGACACCCAGGGCCTTTATGA | |

TABLE 5

| | | SEQ |
|---|---|---|
| | Sequence | ID NO: |
| TGF-β1 | MPPSGLRLLPLLLPLPWLLVLTPGRPAAGLSTSKTIDMELVKRKRIEAIRGQILSKL | 47 |
| | RLASPPSQGEVPPGPLPEAVLALYNSTRDRVAGESADPEPEPEADYYAKEVTRVLMV | |
| | DRNNAIYEKTKDISHSIYMFFNTSDIREAVPEPPLLSRAELRLQRLKSSVEQHVELY | |
| | QKYSNNSWRYLGNRLLTPTDTPEWLSFDVTGVVRQWLNQGDGIQGFRFSAHSSSDSK | |
| | DNKLHVEINGISPKRRGDLGTIHDMNRPFLLLMATPLERAQHLHSSRHRRALDTNYC | |
| | FSSTEKNCCVRQLYTDFRKDLGWKWTHEPKGYHANFCLGPCPYTWSLDTQYSKVLAL | |
| | YNQHNPGASASPCCVPQALEPLPIVYYVGRKPKVEQLSNMIVRSCKCS | |
| | ATGCCGCCCTCGGGGCTGCGGCTACTGCCGCTTCTGCTCCCACTCCCGTGGCTTCTA | 48 |
| | GTGCTGACGCCCGGGAGGCCAGCCGCGGGACTCTCCACCTCTAAGACCATCGACATG | |
| | GAGCTGGTGAAACGGAAGCGCATCGAAGCCATCCGTGGCCAGATCCTGTCCAAACTA | |
| | AGGCTCGCCAGTCCCCCAAGCCAGGGGGAGGTACCGCCCGGCCCGCTGCCCGAGGCG | |
| | GTGCTCGCTTTGTACAACAGCACCCGCGACCGGGTGGCAGGCGAGAGCGCCGACCCA | |
| | GAGCCGGAGCCCGAAGCGGACTACTATGCTAAAGAGGTCACCCGCGTGCTAATGGTG | |
| | GACCGCAACAACGCCATCTATGAGAAAACCAAAGACATCTCACACAGTATATATATG | |
| | TTCTTCAATACGTCAGACATTCGGGAAGCAGTGCCCGAACCCCCATTGCTGTCCCGT | |
| | GCAGAGCTGCGCTTGCAGAGATTAAAATCAAGTGTGGAGCAACATGTGGAACTCTAC | |
| | CAGAAATATAGCAACAATTCCTGGCGTTACCTTGGTAACCGGCTGCTGACCCCCACT | |
| | GATACGCCTGAGTGGCTGTCTTTTGACGTCACTGGAGTTGTACGGCAGTGGCTGAAC | |
| | CAAGGAGACGGAATACAGGGCTTTCGATTCAGCGCTCACTCTTCTTCTGACAGCAAA | |
| | GATAACAAACTCCACGTGGAAATCAACGGGATCAGCCCCAAACGTCGGGGCGACCTG | |
| | GGCACCATCCATGACATGAACCGGCCCTTCCTGCTCCTCATGGCCACCCCCCTGGAA | |
| | AGGGCCCAGCACCTGCACAGCTCACGGCACCGGAGAGCCCTGGATACCAACTATTGC | |
| | TTCAGCTCCACAGAGAAGAACTGCTGTGTGCGGCAGCTGTACATTGACTTTAGGAAG | |
| | GACCTGGGTTGGAAGTGGATCCACGAGCCCAAGGGCTACCATGCCAACTTCTGTCTG | |
| | GGACCCTGCCCCTATATTTGGAGCCTGGACACACAGTACAGCAAGGTCCTTGCCCTC | |
| | TACAACCAACACAACCCGGGCGCTTCGGCGTCACCGTGCTGCGTGCCGCAGGCTTTG | |
| | GAGCCACTGCCCATCGTCTACTACGTGGGTCGCAAGCCCAAGGTGGAGCAGTTGTCC | |
| | AACATGATTGTGCGCTCCTGCAAGTGCAGCTGA | |
| MFG-E8 | ASGDFCDSSLCLNGGTCLTGQDNDIYCLCPEGFTGLVCNETERGPCSPNPCYNDAKC | 49 |
| (from which | LVTLDTQRGEIFTEYICQCPVGYSGIHCETETNYYNLDGEYMFTTAVPNTAVPTPAP | |
| signal peptide | TPDLSNNLASRCSTQLGMEGGAIADSQISASSVYMGFMGLQRWGPELARLYRTGIVN | |
| is removed) | AWTASNYDSKPWIQVNLLRKMRVSGVMTQGASRAGRAEYLKTFKVAYSLDGRKFEFI | |
| | QDESGGDKEFLGNLDNNSLKVNMFNPTLEAQYIKLYPVSCHRGCTLRFELLGCELHG | |
| | CSEPLGLKNNTIPDSQMSASSSYKTWNLRAFGWYPHLGRLDNQGKINAWTAQSNSAK | |
| | EWLQVDLGTQRQVTGIITQGARDFGHIQYVASYKVAHSDDGVQWTVYEEQGSSKVFQ | |
| | GNLDNNSHKKNIFEKPFMARYVRVLPVSWHNRITLRLELLGC | |
| | GCGTCTGGTGACTTCTGTGACTCCAGCCTGTGCCTGAACGGTGGCACCTGCTTGACG | 50 |
| | GGCCAAGACAATGACATCTACTGCCTCTGCCCTGAAGGCTTCACAGGCCTTGTGTGC | |
| | AATGAGACTGAGAGAGGACCATGCTCCCCAAACCCTTGCTACAATGATGCCAAATGT | |
| | CTGGTGACTTTGGACACACAGCGTGGGGAAATCTTCACCGAATACATCTGCCAGTGC | |
| | CCTGTGGGCTACTCGGGCATCCACTGTGAAACCGAGACCAACTACTACAACCTGGAT | |
| | GGAGAATACATGTTCACCACAGCCGTCCCCAATACTGCCGTCCCCACCCCGGCCCCC | |
| | ACCCCCGATCTTTCCAACAACCTAGCCTCCCGTTGTTCTACACAGCTGGGCATGGAA | |
| | GGGGGCGCCATTGCTGATTCACAGATTTCCGCCTCGTCTGTGTATATGGGTTTCATG | |
| | GGCTTGCAGCGCTGGGGCCCGGAGCTGGCTCGTCTGTACCGCACAGGGATCGTCAAT | |
| | GCCTGGACAGCCAGCAACTATGATAGCAAGCCCTGGATCCAGGTGAACCTTCTGCGG | |
| | AAGATGCGGGTATCAGGTGTGATGACGCAGGGTGCCAGCCGTGCCGGGAGGGCGGAG | |
| | TACCTGAAGACCTTCAAGGTGGCTTACAGCCTCGACGGACGCAAGTTTGAGTTCATC | |
| | CAGGATGAAAGCGGTGGAGACAAGGAGTTTTTGGGTAACCTGGACAACAACAGCCTG | |
| | AAGGTTAACATGTTCAACCCGACTCTGGAGGCACAGTACATAAAGCTGTACCCTGTT | |
| | TCGTGCCACCGCGGCTGCACCCTCCGCTTCGAGCTCCTGGGCTGTGAGTTGCACGGA | |
| | TGTTCTGAGCCCCTGGGCCTGAAGAATAACACAATTCCTGACAGCCAGATGTCAGCC | |
| | TCCAGCAGCTACAAGACATGGAACCTGCGTGCTTTTGGCTGGTACCCCCACTTGGGA | |
| | AGGCTGGATAATCAGGGCAAGATCAATGCCTGGACGGCTCAGAGCAACAGTGCCAAG | |
| | GAATGGCTGCAGGTTGACCTGGGCACTCAGAGGCAAGTGACAGGAATCATCACCCAG | |
| | GGGGCCCGTGACTTTGGCCACATCCAGTATGTGGCGTCCTACAAGGTAGCCCACAGT | |
| | GATGATGGTGTGCAGTGGACTGTATATGAGGAGCAAGGAAGCAGCAAGGTCTTCCAG | |
| | GGCAACTTGGACAACAACTCCCACAAGAAGAACATCTTCGAGAAACCCTTCATGGCT | |
| | CGCTACGTGCGTGTCCTTCCAGTGTCCTGGCATAACCGCATCACCCTGCGCCTGGAG | |
| | CTGCTGGGCTGTTAA | |
| TGF-β1- | MPPSGLRLLPLLLPLPWLLVLTPGRPAAGLSTSKTIDMELVKRKRIEAIRGQILSKL | 51 |
| MFG-E8 | RLASPPSQGEVPPGPLPEAVLALYNSTRDRVAGESADPEPEPEADYYAKEVTRVLMV | |
| (TGF-β1 + | DRNNAIYEKTKDISHSIYMFFNTSDIREAVPEPPLLSRAELRLQRLKSSVEQHVELY | |
| peptide linker | QKYSNNSWRYLGNRLLTPTDTPEWLSFDVTGVVRQWLNQGDGIQGFRFSAHSSSDSK | |
| 3 + MFG-E8 | DNKLHVEINGISPKRRGDLGTIHDMNRPFLLLMATPLERAQHLHSSRHRRALDTNYC | |
| (from which | FSSTEKNCCVRQLYIDFRKDLGWKWIHEPKGYHANFCLGPCPYIWSLDTQYSKVLAL | |
| signal peptide | YNQHNPGASASPCCVPQALEPLPIVYYVGRKPKVEQLSNMIVRSCKCSGGGGSASGD | |
| is removed)) | FCDSSLCLNGGTCLTGQDNDIYCLCPEGFTGLVCNETERGPCSPNPCYNDAKCLVTL | |
| | DTQRGEIFTEYICQCPVGYSGIHCETETNYYNLDGEYMFTTAVPNTAVPTPAPTPDL | |
| | SNNLASRCSTOLGMEGGAIADSQISASSVYMGFMGLQRWGPELARLYRTGIVNAWTA | |
| | SNYDSKPWIQVNLLRKMRVSGVMTQGASRAGRAEYLKTFKVAYSLDGRKFEFIQDES | |
| | GGDKEFLGNLDNNSLKVNMFNPTLEAQYIKLYPVSCHRGCTLRFELLGCELHGCSEP | |
| | LGLKNNTIPDSQMSASSSYKTWNLRAFGWYPHLGRLDNQGKINAWTAQSNSAKEWLQ | |
| | VDLGTQRQVTGIITQGARDFGHIQYVASYKVAHSDDGVQWTVYEEQGSSKVFQGNLD | |
| | NNSHKKNIFEKPFMARYVRVLPVSWHNRITLRLELLGC | |
| | ATGCCGCCCTCGGGGCTGCGGCTACTGCCGCTTCTGCTCCCACTCCCGTGGCTTCTA | 52 |
| | GTGCTGACGCCCGGGAGGCCAGCCGCGGGACTCTCCACCTCTAAGACCATCGACATG | |
| | GAGCTGGTGAAACGGAAGCGCATCGAAGCCATCCGTGGCCAGATCCTGTCCAAACTA | |
| | AGGCTCGCCAGTCCCCCAAGCCAGGGGGAGGTACCGCCCGGCCCGCTGCCCGAGGCG | |
| | GTGCTCGCTTTGTACAACAGCACCCGCGACCGGGTGGCAGGCGAGAGCGCCGACCCA | |
| | GAGCCGGAGCCCGAAGCGGACTACTATGCTAAAGAGGTCACCCGCGTGCTAATGGTG | |
| | GACCGCAACAACGCCATCTATGAGAAAACCAAAGACATCTCACACAGTATATATATG | |
| | TTCTTCAATACGTCAGACATTCGGGAAGCAGTGCCCGAACCCCCATTGCTGTCCCGT | |
| | GCAGAGCTGCGCTTGCAGAGATTAAAATCAAGTGTGGAGCAACATGTGGAACTCTAC | |
| | CAGAAATATAGCAACAATTCCTGGCGTTACCTTGGTAACCGGCTGCTGACCCCCACT | |
| | GATACGCCTGAGTGGCTGTCTTTTGACGTCACTGGAGTTGTACGGCAGTGGCTGAAC | |
| | CAAGGAGACGGAATACAGGGCTTTCGATTCAGCGCTCACTCTTCTTCTGACAGCAAA | |
| | GATAACAAACTCCACGTGGAAATCAACGGGATCAGCCCCAAACGTCGGGGCGACCTG | |
| | GGCACCATCCATGACATGAACCGGCCCTTCCTGCTCCTCATGGCCACCCCCCTGGAA | |
| | AGGGCCCAGCACCTGCACAGCTCACGGCACCGGAGAGCCCTGGATACCAACTATTGC | |
| | TTCAGCTCCACAGAGAAGAACTGCTGTGTGCGGCAGCTGTACATTGACTTTAGGAAG | |
| | GACCTGGGTTGGAAGTGGATCCACGAGCCCAAGGGCTACCATGCCAACTTCTGTCTG | |
| | GGACCCTGCCCCTATATTTGGAGCCTGGACACACAGTACAGCAAGGTCCTTGCCCTC | |
| | TACAACCAACACAACCCGGGCGCTTCGGCGTCACCGTGCTGCGTGCCGCAGGCTTTG | |
| | GAGCCACTGCCCATCGTCTACTACGTGGGTCGCAAGCCCAAGGTGGAGCAGTTGTCC | |
| | AACATGATTGTGCGCTCCTGCAAGTGCAGCGGAGGAGGAGGAAGCGCGTCTGGTGAC | |
| | TTCTGTGACTCCAGCCTGTGCCTGAACGGTGGCACCTGCTTGACGGGCCAAGACAAT | |
| | GACATCTACTGCCTCTGCCCTGAAGGCTTCACAGGCCTTGTGTGCAATGAGACTGAG | |
| | AGAGGACCATGCTCCCCAAACCCTTGCTACAATGATGCCAAATGTCTGGTGACTTTG | |
| | GACACACAGCGTGGGGAAATCTTCACCGAATACATCTGCCAGTGCCCTGTGGGCTAC | |
| | TCGGGCATCCACTGTGAAACCGAGACCAACTACTACAACCTGGATGGAGAATACATG | |
| | TTCACCACAGCCGTCCCCAATACTGCCGTCCCCACCCCGGCCCCCACCCCCGATCTT | |
| | TCCAACAACCTAGCCTCCCGTTGTTCTACACAGCTGGGCATGGAAGGGGGCGCCATT | |
| | GCTGATTCACAGATTTCCGCCTCGTCTGTGTATATGGGTTTCATGGGCTTGCAGCGC | |
| | TGGGGCCCGGAGCTGGCTCGTCTGTACCGCACAGGGATCGTCAATGCCTGGACAGCC | |
| | AGCAACTATGATAGCAAGCCCTGGATCCAGGTGAACCTTCTGCGGAAGATGCGGGTA | |
| | TCAGGTGTGATGACGCAGGGTGCCAGCCGTGCCGGGAGGGCGGAGTACCTGAAGACC | |
| | TTCAAGGTGGCTTACAGCCTCGACGGACGCAAGTTTGAGTTCATCCAGGATGAAAGC | |
| | GGTGGAGACAAGGAGTTTTTGGGTAACCTGGACAACAACAGCCTGAAGGTTAACATG | |
| | TTCAACCCGACTCTGGAGGCACAGTACATAAAGCTGTACCCTGTTTCGTGCCACCGC | |
| | GGCTGCACCCTCCGCTTCGAGCTCCTGGGCTGTGAGTTGCACGGATGTTCTGAGCCC | |
| | CTGGGCCTGAAGAATAACACAATTCCTGACAGCCAGATGTCAGCCTCCAGCAGCTAC | |
| | AAGACATGGAACCTGCGTGCTTTTGGCTGGTACCCCCACTTGGGAAGGCTGGATAAT | |
| | CAGGGCAAGATCAATGCCTGGACGGCTCAGAGCAACAGTGCCAAGGAATGGCTGCAG | |
| | GTTGACCTGGGCACTCAGAGGCAAGTGACAGGAATCATCACCCAGGGGGCCCGTGAC | |
| | TTTGGCCACATCCAGTATGTGGCGTCCTACAAGGTAGCCCACAGTGATGATGGTGTG | |
| | CAGTGGACTGTATATGAGGAGCAAGGAAGCAGCAAGGTCTTCCAGGGCAACTTGGAC | |
| | AACAACTCCCACAAGAAGAACATCTTCGAGAAACCCTTCATGGCTCGCTACGTGCGT | |
| | GTCCTTCCAGTGTCCTGGCATAACCGCATCACCCTGCGCCTGGAGCTGCTGGGCTGT | |
| | TAA | |

TABLE 6

| | | SEQ |
|---|---|---|
| | Sequence | ID NO: |
| IL-4 | HIHGCDKNHLREIIGILNEVTGEGTPCTEMDVPNVLTATKNTTESELVCRASKVLRI | 53 |
| (from which | FYLKHGKTPCLKKNSSVLMELQRLFRAFRCLDSSISCTMNESKSTSLKDFLESLKSI | |
| signal peptide | MQMDYS | |
| is removed) | CATATCCACGGATGCGACAAAAATCACTTGAGAGAGATCATCGGCATTTTGAACGAG | 54 |
| | GTCACAGGAGAAGGGACGCCATGCACGGAGATGGATGTGCCAAACGTCCTCACAGCA | |
| | ACGAAGAACACCACAGAGAGTGAGCTCGTCTGTAGGGCTTCCAAGGTGCTTCGCATA | |
| | TTTTATTTAAAACATGGGAAAACTCCATGCTTGAAGAAGAACTCTAGTGTTCTCATG | |
| | GAGCTGCAGAGACTCTTTCGGGCTTTTCGATGCCTGGATTCATCGATAAGCTGCACC | |
| | ATGAATGAGTCCAAGTCCACATCACTGAAAGACTTCCTGGAAAGCCTAAAGAGCATC | |
| | ATGCAAATGGATTACTCGTAG | |
| CD81 | MGVEGCTKCIKYLLFVFNFVFWLAGGVILGVALWLRHDPQTTSLLYLELGNKPAPNT | 61 |
| (amino acids | FYVGIYILIAVGAVMMFVGFLGCYGAIQESQCLLGTFFTCLVILFACEVAAGIWGFV | |
| 1 to 177) | NKDQIAKDVKQFYDQALQQAVMDDDANNAKAVVKTFHETLNCCGSNALTTLTTTILR | |
| | NSLCPS | |
| | ATGGGGGTGGAGGGCTGCACCAAATGCATCAAATACCTGCTCTTCGTCTTCAATTTC | 62 |
| | GTCTTCTGGCTGGCTGGAGGCGTGATCCTAGGTGTAGCTCTGTGGTTGCGTCATGAT | |
| | CCACAGACCACCAGCCTGCTGTACCTGGAACTGGGAAACAAACCGGCACCCAACACC | |
| | TTCTACGTGGGCATCTACATTCTCATTGCTGTGGGAGCTGTGATGATGTTTGTAGGC | |
| | TTCCTGGGGTGCTATGGGGCCATCCAGGAGTCCCAGTGTCTGCTGGGGACGTTCTTC | |
| | ACCTGCCTTGTGATCCTGTTTGCCTGTGAGGTGGCTGCAGGCATCTGGGGCTTCGTA | |
| | AACAAAGACCAGATCGCCAAGGATGTGAAGCAGTTCTATGACCAGGCCCTTCAGCAA | |
| | GCTGTGATGGATGATGATGCCAACAATGCCAAGGCTGTGGTGAAGACTTTCCATGAG | |
| | ACGCTCAACTGTTGTGGCTCCAACGCACTGACCACACTGACTACCACCATACTGAGG | |
| | AACAGCCTGTGTCCCTCA | |
| CD81 | GGNILTPLLQQDCHQKIDELFSGKLYLIGIAAIVVAVIMIFEMILSMVLCCGIRNSS | 63 |
| (amino acids | VY | |
| 178 to 236) | GGCGGCAACATACTCACCCCCTTACTGCAGCAAGATTGTCATCAGAAAATCGATGAG | 64 |
| | CTCTTCTCTGGGAAGCTGTACCTCATTGGAATTGCAGCCATTGTGGTAGCTGTCATT | |
| | ATGATCTTTGAGATGATTCTGAGCATGGTGCTGTGCTGTGGCATCCGGAACAGCTCC | |
| | GTGTACTGA | |
| CD81-IL-4 | MGVEGCTKCIKYLLFVFNFVFWLAGGVILGVALWLRHDPQTTSLLYLELGNKPAPNT | 55 |
| | FYVGIYILIAVGAVMMFVGFLGCYGAIQESQCLLGTFFTCLVILFACEVAAGIWGFV | |
| | NKDQIAKDVKQFYDQALQQAVMDDDANNAKAVVKTFHETLNCCGSNALTTLTTTILR | |
| | NSLCPSGGGGSHIHGCDKNHLREIIGILNEVTGEGTPCTEMDVPNVLTATKNTTESE | |
| | LVCRASKVLRIFYLKHGKTPCLKKNSSVLMELQRLFRAFRCLDSSISCTMNESKSTS | |
| | LKDFLESLKSIMQMDYSGGGGSGGNILTPLLQQDCHQKIDELFSGKLYLIGIAAIVV | |
| | AVIMIFEMILSMVLCCGIRNSSVY | |
| | ATGGGGGTGGAGGGCTGCACCAAATGCATCAAATACCTGCTCTTCGTCTTCAATTTC | 56 |
| | GTCTTCTGGCTGGCTGGAGGCGTGATCCTAGGTGTAGCTCTGTGGTTGCGTCATGAT | |
| | CCACAGACCACCAGCCTGCTGTACCTGGAACTGGGAAACAAACCGGCACCCAACACC | |
| | TTCTACGTGGGCATCTACATTCTCATTGCTGTGGGAGCTGTGATGATGTTTGTAGGC | |
| | TTCCTGGGGTGCTATGGGGCCATCCAGGAGTCCCAGTGTCTGCTGGGGACGTTCTTC | |
| | ACCTGCCTTGTGATCCTGTTTGCCTGTGAGGTGGCTGCAGGCATCTGGGGCTTCGTA | |
| | AACAAAGACCAGATCGCCAAGGATGTGAAGCAGTTCTATGACCAGGCCCTTCAGCAA | |
| | GCTGTGATGGATGATGATGCCAACAATGCCAAGGCTGTGGTGAAGACTTTCCATGAG | |
| | ACGCTCAACTGTTGTGGCTCCAACGCACTGACCACACTGACTACCACCATACTGAGG | |
| | AACAGCCTGTGTCCCTCAGGAGGAGGAGGAAGCCATATCCACGGATGCGACAAAAAT | |
| | CACTTGAGAGAGATCATCGGCATTTTGAACGAGGTCACAGGAGAAGGGACGCCATGC | |
| | ACGGAGATGGATGTGCCAAACGTCCTCACAGCAACGAAGAACACCACAGAGAGTGAG | |
| | CTCGTCTGTAGGGCTTCCAAGGTGCTTCGCATATTTTATTTAAAACATGGGAAAACT | |
| | CCATGCTTGAAGAAGAACTCTAGTGTTCTCATGGAGCTGCAGAGACTCTTTCGGGCT | |
| | TTTCGATGCCTGGATTCATCGATAAGCTGCACCATGAATGAGTCCAAGTCCACATCA | |
| | CTGAAAGACTTCCTGGAAAGCCTAAAGAGCATCATGCAAATGGATTACTCGGGAGGA | |
| | GGAGGAAGCGGCGGCAACATACTCACCCCCTTACTGCAGCAAGATTGTCATCAGAAA | |
| | ATCGATGAGCTCTTCTCTGGGAAGCTGTACCTCATTGGAATTGCAGCCATTGTGGTA | |
| | GCTGTCATTATGATCTTTGAGATGATTCTGAGCATGGTGCTGTGCTGTGGCATCCGG | |
| | AACAGCTCCGTGTACTGA | |

TABLE 7

| | | SEQ |
|---|---|---|
| | Sequence | ID NO: |
| Signal peptide | MALQIPSLLLSAAVVVLMVLSSPGTEG | 33 |
| of MHC class | ATGGCTCTGCAGATCCCCAGCCTCCTCCTCTCGGCTGCTGTGGTGGTGCTGATGGTGCTGAGC | 34 |
| IIß chain | AGCCCAGGGACTGAGGGC | |
| OVA | ISQAVHAAHAEINEAGR | 35 |
| peptide 2 | ATATCTCAAGCTGTCCATGCAGCACATGCAGAAATCAATGAAGCAGGCAGA | |
| (for MHC class | | |
| II molecule) | | |
| MHC class | GDSERHFVYQFMGECYFTNGTQRIRYVTRYIYNREEYVRYDSDVGEHRAVTELGRPDAEYWNS | 36 |
| IIß chain | QPEILERTRAELDTVCRHNYEGPETHTSLRRLEQPNVVISLSRTEALNHHNTLVCSVTDFYPA | |
| (from which | KIKVRWFRNGQEETVGVSSTQLIRNGDWTFQVLVMLEMTPRRGEVYTCHVEHPSLKSPITVEW | |
| signal peptide | RAQSESAWSKMLSGIGGCVLGVIFLGLGLFIRHRSQKGPRGPPPAGLLQ | |
| is removed) | GGAGACTCCGAAAGGCATTTCGTGTACCAGTTCATGGGCGAGTGCTACTTCACCAACGGGACG | 37 |
| | CAGCGCATACGATATGTGACCAGATACATCTACAACCGGGAGGAGTACGTGCGCTACGACAGC | |
| | GACGTGGGCGAGCACCGCGCGGTGACCGAGCTGGGGCGGCCAGACGCCGAGTACTGGAACAGC | |
| | CAGCCGGAGATCCTGGAGCGAACGCGGGCCGAGCTGGACACGGTGTGCAGACACAACTACGAG | |
| | GGGCCGGAGACCCACACCTCCCTGCGGCGGCTTGAACAGCCCAATGTCGTCATCTCCCTGTCC | |
| | AGGACAGAGGCCCTCAACCACCACAACACTCTGGTCTGCTCAGTGACAGATTTCTACCCAGCC | |
| | AAGATCAAAGTGCGCTGGTTCCGGAATGGCCAGGAGGAGACGGTGGGGGTCTCATCCACACAG | |
| | CTTATTAGGAATGGGGACTGGACCTTCCAGGTCCTGGTCATGCTGGAGATGACCCCTCGGCGG | |
| | GGAGAGGTCTACACCTGTCACGTGGAGCATCCCAGCCTGAAGAGCCCCATCACTGTGGAGTGG | |
| | AGGGCACAGTCTGAGTCTGCCTGGAGCAAGATGTTGAGCGGCATCGGGGGCTGCGTGCTTGGG | |
| | GTGATCTTCCTCGGGCTTGGCCTTTTCATCCGTCACAGGAGTCAGAAAGGACCTCGAGGCCCT | |
| | CCTCCAGCAGGGCTCCTGCAG | |
| Peptide linker 4 | GGGGSGGGGSG | 39 |
| | GGAGGAGGAGGAAGCGGAGGAGGAGGAAGCGGA | 40 |
| sc-Dimer | MALQIPSLLLSAAVVVLMVLSSPGTEGISQAVHAAHAEINEAGRGGGGSGGGGSGGDSERHFV | 41 |
| (OVA peptide 2 + | YQFMGECYFTNGTQRIRYVTRYIYNREEYVRYDSDVGEHRAVTELGRPDAEYWNSQPEILERT | |
| peptide linker | RAELDTVCRHNYEGPETHTSLRRLEQPNVVISLSRTEALNHHNTLVCSVTDFYPAKIKVRWFR | |
| 4 + MHC class | NGQEETVGVSSTQLIRNGDWTFQVLVMLEMTPRRGEVYTCHVEHPSLKSPITVEWRAQSESAW | |
| IIß chain | SKMLSGIGGCVLGVIFLGLGLFIRHRSQKGPRGPPPAGLLQ | |
| (from which | ATGGCTCTGCAGATCCCCAGCCTCCTCCTCTCGGCTGCTGTGGTGGTGCTGATGGTGCTGAGC | 42 |
| signal peptide | AGCCCAGGGACTGAGGGCATATCTCAAGCTGTCCATGCAGCACATGCAGAAATCAATGAAGCA | |
| is removed)) | GGCAGAGGAGGAGGAGGAAGCGGAGGAGGAGGAAGCGGAGGAGACTCCGAAAGGCATTTCGTG | |
| | TACCAGTTCATGGGCGAGTGCTACTTCACCAACGGGACGCAGCGCATACGATATGTGACCAGA | |
| | TACATCTACAACCGGGAGGAGTACGTGCGCTACGACAGCGACGTGGGCGAGCACCGCGCGGTG | |
| | ACCGAGCTGGGGCGGCCAGACGCCGAGTACTGGAACAGCCAGCCGGAGATCCTGGAGCGAACG | |
| | CGGGCCGAGCTGGACACGGTGTGCAGACACAACTACGAGGGGCCGGAGACCCACACCTCCCTG | |
| | CGGCGGCTTGAACAGCCCAATGTCGTCATCTCCCTGTCCAGGACAGAGGCCCTCAACCACCAC | |
| | AACACTCTGGTCTGCTCAGTGACAGATTTCTACCCAGCCAAGATCAAAGTGCGCTGGTTCCGG | |
| | AATGGCCAGGAGGAGACGGTGGGGGTCTCATCCACACAGCTTATTAGGAATGGGGACTGGACC | |
| | TTCCAGGTCCTGGTCATGCTGGAGATGACCCCTCGGCGGGGAGAGGTCTACACCTGTCACGTG | |
| | GAGCATCCCAGCCTGAAGAGCCCCATCACTGTGGAGTGGAGGGCACAGTCTGAGTCTGCCTGG | |
| | AGCAAGATGTTGAGCGGCATCGGGGGCTGCGTGCTTGGGGTGATCTTCCTCGGGCTTGGCCTT | |
| | TTCATCCGTCACAGGAGTCAGAAAGGACCTCGAGGCCCTCCTCCAGCAGGGCTCCTGCAG | |
| sc-Dimer- | MALQIPSLLLSAAVVVLMVLSSPGTEGISQAVHAAHAEINEAGRGGGGSGGGGSGGDSERHFV | 91 |
| CD81- | YQFMGECYFTNGTQRIRYVTRYIYNREEYVRYDSDVGEHRAVTELGRPDAEYWNSQPEILERT | |
| IL-12α | RAELDTVCRHNYEGPETHTSLRRLEQPNVVISLSRTEALNHHNTLVCSVTDFYPAKIKVRWFR | |
| | NGQEETVGVSSTQLIRNGDWTFQVLVMLEMTPRRGEVYTCHVEHPSLKSPITVEWRAQSESAW | |
| | SKMLSGIGGCVLGVIFLGLGLFIRHRSQKGPRGPPPAGLLQMGVEGCTKCIKYLLFVFNFVFW | |
| | LAGGVILGVALWLRHDPQTTSLLYLELGNKPAPNTFYVGIYILIAVGAVMMFVGFLGCYGAIQ | |
| | ESQCLLGTFFTCLVILFACEVAAGIWGFVNKDQIAKDVKQFYDQALQQAVMDDDANNAKAVVK | |
| | TFHETLNCCGSNALTTLTTTILRNSLCPSGGGGSRVIPVSGPARCLSQSRNLLKTTDDMVKTA | |
| | REKLKHYSCTAEDIDHEDITRDQTSTLKTCLPLELHKNESCLATRETSSTTRGSCLPPQKTSL | |
| | MMTLCLGSIYEDLKMYQTEFQAINAALQNHNHQQIILDKGMLVAIDELMQSLNHNGETLRQKP | |
| | PVGEADPYRVKMKLCILLHAFSTRVVTINRVMGYLSSAGGGGSGGNILTPLLQQDCHQKIDEL | |
| | FSGKLYLIGIAAIVVAVIMIFEMILSMVLCCGIRNSSVY | |
| | ATGGCTCTGCAGATCCCCAGCCTCCTCCTCTCGGCTGCTGTGGTGGTGCTGATGGTGCTGAGC | |
| | AGCCCAGGGACTGAGGGCATATCTCAAGCTGTCCATGCAGCACATGCAGAAATCAATGAAGCA | 92 |
| | GGCAGAGGAGGAGGAGGAAGCGGAGGAGGAGGAAGCGGAGGAGACTCCGAAAGGCATTTCGTG | |
| | TACCAGTTCATGGGCGAGTGCTACTTCACCAACGGGACGCAGCGCATACGATATGTGACCAGA | |
| | TACATCTACAACCGGGAGGAGTACGTGCGCTACGACAGCGACGTGGGCGAGCACCGCGCGGTG | |
| | ACCGAGCTGGGGCGGCCAGACGCCGAGTACTGGAACAGCCAGCCGGAGATCCTGGAGCGAACG | |
| | CGGGCCGAGCTGGACACGGTGTGCAGACACAACTACGAGGGGCCGGAGACCCACACCTCCCTG | |
| | CGGCGGCTTGAACAGCCCAATGTCGTCATCTCCCTGTCCAGGACAGAGGCCCTCAACCACCAC | |
| | AACACTCTGGTCTGCTCAGTGACAGATTTCTACCCAGCCAAGATCAAAGTGCGCTGGTTCCGG | |
| | AATGGCCAGGAGGAGACGGTGGGGGTCTCATCCACACAGCTTATTAGGAATGGGGACTGGACC | |
| | TTCCAGGTCCTGGTCATGCTGGAGATGACCCCTCGGCGGGGAGAGGTCTACACCTGTCACGTG | |
| | GAGCATCCCAGCCTGAAGAGCCCCATCACTGTGGAGTGGAGGGCACAGTCTGAGTCTGCCTGG | |
| | AGCAAGATGTTGAGCGGCATCGGGGGCTGCGTGCTTGGGGTGATCTTCCTCGGGCTTGGCCTT | |
| | TTCATCCGTCACAGGAGTCAGAAAGGACCTCGAGGCCCTCCTCCAGCAGGGCTCCTGCAGATG | |
| | GGGGTGGAGGGCTGCACCAAATGCATCAAATACCTGCTCTTCGTCTTCAATTTCGTCTTCTGG | |
| | CTGGCTGGAGGCGTGATCCTAGGTGTAGCTCTGTGGTTGCGTCATGATCCACAGACCACCAGC | |
| | CTGCTGTACCTGGAACTGGGAAACAAACCGGCACCCAACACCTTCTACGTGGGCATCTACATT | |
| | CTCATTGCTGTGGGAGCTGTGATGATGTTTGTAGGCTTCCTGGGGTGCTATGGGGCCATCCAG | |
| | GAGTCCCAGTGTCTGCTGGGGACGTTCTTCACCTGCCTTGTGATCCTGTTTGCCTGTGAGGTG | |
| | GCTGCAGGCATCTGGGGCTTCGTAAACAAAGACCAGATCGCCAAGGATGTGAAGCAGTTCTAT | |
| | GACCAGGCCCTTCAGCAAGCTGTGATGGATGATGATGCCAACAATGCCAAGGCTGTGGTGAAG | |
| | ACTTTCCATGAGACGCTCAACTGTTGTGGCTCCAACGCACTGACCACACTGACTACCACCATA | |
| | CTGAGGAACAGCCTGTGTCCCTCAGGAGGAGGAGGAAGCAGGGTCATTCCAGTCTCTGGACCT | |
| | GCCAGGTGTCTTAGCCAGTCCCGAAACCTGCTGAAGACCACAGATGACATGGTGAAGACGGCC | |
| | AGAGAAAAACTGAAACATTATTCCTGCACTGCTGAAGACATCGATCATGAAGACATCACACGG | |
| | GACCAAACCAGCACATTGAAGACCTGTTTACCACTGGAACTACACAAGAACGAGAGTTGCCTG | |
| | GCTACTAGAGAGACTTCTTCCACAACAAGAGGGAGCTGCCTGCCCCCACAGAAGACGTCTTTG | |
| | ATGATGACCCTGTGCCTTGGTAGCATCTATGAGGACTTGAAGATGTACCAGACAGAGTTCCAG | |
| | GCCATCAACGCAGCACTTCAGAATCACAACCATCAGCAGATCATTCTAGACAAGGGCATGCTG | |
| | GTGGCCATCGATGAGCTGATGCAGTCTCTGAATCATAATGGCGAGACTCTGCGCCAGAAACCT | |
| | CCTGTGGGAGAAGCAGACCCTTACAGAGTGAAAATGAAGCTCTGCATCCTGCTTCACGCCTTC | |
| | AGCACCCGCGTCGTGACCATCAACAGGGTGATGGGCTATCTGAGCTCCGCCGGAGGAGGAGGA | |
| | AGCGGCGGCAACATACTCACCCCCTTACTGCAGCAAGATTGTCATCAGAAAATCGATGAGCTC | |
| | TTCTCTGGGAAGCTGTACCTCATTGGAATTGCAGCCATTGTGGTAGCTGTCATTATGATCTTT | |
| | GAGATGATTCTGAGCATGGTGCTGTGCTGTGGCATCCGGAACAGCTCCGTGTACTGA | |
| MHC class | MPRSRALILGVLALTTMLSLCGGEDDIEADHVGTYGISVYQSPGDIGQYTFEFDGDELFYVDL | 45 |
| IIα chain | DKKETVWMLPEFGQLASFDPQGGLQNIAVVKHNLGVLTKRSNSTPATNEAPQATVFPKSPVLL | |
| | GQPNTLICFVDNIFPPVINITWLRNSKSVADGVYETSFFVNRDYSFHKLSYLTFIPSDDDIYD | |
| | CKVEHWGLEEPVLKHWEPEIPAPMSELTETVVCALGLSVGLVGIVVGTIFIIQGLRSGGTSRH | |
| | PGPL | |
| | ATGCCGCGCAGCAGAGCTCTGATTCTGGGGGTCCTCGCCCTGACCACCATGCTCAGCCTCTGT | 46 |
| | GGAGGTGAAGACGACATTGAGGCCGACCACGTAGGCACCTATGGTATAAGTGTATATCAGTCT | |
| | CCTGGAGACATTGGCCAGTACACATTTGAATTTGATGGTGATGAGTTGTTCTATGTGGACTTG | |
| | GATAAGAAGGAGACTGTCTGGATGCTTCCTGAGTTTGGCCAATTGGCAAGCTTTGACCCCCAA | |
| | GGTGGACTGCAAAACATAGCTGTAGTAAAACACAACTTGGGAGTCTTGACTAAGAGGTCAAAT | |
| | TCCACCCCAGCTACCAATGAGGCTCCTCAAGCGACTGTGTTCCCCAAGTCCCCTGTGCTGCTG | |
| | GGTCAGCCCAACACCCTCATCTGCTTTGTGGACAACATCTTCCCTCCTGTGATCAACATCACA | |
| | TGGCTCAGAAATAGCAAGTCAGTCGCAGACGGTGTTTATGAGACCAGCTTCTTCGTCAACCGT | |
| | GACTATTCCTTCCACAAGCTGTCTTATCTCACCTTCATCCCTTCTGACGATGACATTTATGAC | |
| | TGCAAGGTGGAACACTGGGGCCTGGAGGAGCCGGTTCTGAAACACTGGGAACCTGAGATTCCA | |
| | GCCCCCATGTCAGAGCTGACAGAGACTGTGGTCTGTGCCCTGGGGTTGTCTGTGGGCCTTGTG | |
| | GGCATCGTGGTGGGCACCATCTTCATCATTCAAGGCCTGCGATCAGGTGGCACCTCCAGACAC | |
| | CCAGGGCCTTTATGA | |

TABLE 8

| | | SEQ |
|---|---|---|
| | Sequence | ID NO: |
| IL-12α | RVIPVSGPARCLSQSRNLLKTTDDMVKTAREKLKHYSCTAEDIDHEDITRDQTSTLKTCLPLE | 93 |
| (from which | LHKNESCLATRETSSTTRGSCLPPQKTSLMMTLCLGSIYEDLKMYQTEFQAINAALQNHNHQQ | |
| signal peptide | IILDKGMLVAIDELMQSLNHNGETLRQKPPVGEADPYRVKMKLCILLHAFSTRVVTINRVMGY | |
| is removed) | LSSA | |
| | AGGGTCATTCCAGTCTCTGGACCTGCCAGGTGTCTTAGCCAGTCCCGAAACCTGCTGAAGACC | 94 |
| | ACAGATGACATGGTGAAGACGGCCAGAGAAAAACTGAAACATTATTCCTGCACTGCTGAAGAC | |
| | ATCGATCATGAAGACATCACACGGGACCAAACCAGCACATTGAAGACCTGTTTACCACTGGAA | |
| | CTACACAAGAACGAGAGTTGCCTGGCTACTAGAGAGACTTCTTCCACAACAAGAGGGAGCTGC | |
| | CTGCCCCCACAGAAGACGTCTTTGATGATGACCCTGTGCCTTGGTAGCATCTATGAGGACTTG | |
| | AAGATGTACCAGACAGAGTTCCAGGCCATCAACGCAGCACTTCAGAATCACAACCATCAGCAG | |
| | ATCATTCTAGACAAGGGCATGCTGGTGGCCATCGATGAGCTGATGCAGTCTCTGAATCATAAT | |
| | GGCGAGACTCTGCGCCAGAAACCTCCTGTGGGAGAAGCAGACCCTTACAGAGTGAAAATGAAG | |
| | CTCTGCATCCTGCTTCACGCCTTCAGCACCCGCGTCGTGACCATCAACAGGGTGATGGGCTAT | |
| | CTGAGCTCCGCC | |
| CD81 | MGVEGCTKCIKYLLFVFNFVFWLAGGVILGVALWLRHDPQTTSLLYLELGNKPAPNTFYVGIY | 61 |
| (amino acids | ILIAVGAVMMFVGFLGCYGAIQESQCLLGTFFTCLVILFACEVAAGIWGFVNKDQIAKDVKQF | |
| 1 to 177) | YDQALQQAVMDDDANNAKAVVKTFHETLNCCGSNALTTLTTTILRNSLCPS | |
| | ATGGGGGTGGAGGGCTGCACCAAATGCATCAAATACCTGCTCTTCGTCTTCAATTTCGTCTTC | 62 |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCA | |
| CD81 | GGNILTPLLQQDCHQKIDELFSGKLYLIGIAAIVVAVIMIFEMILSMVLCCGIRNSSVY | 63 |
| (amino acids | GGCGGCAACATACTCACCCCCTTACTGCAGCAAGATTGTCATCAGAAAATCGATGAGCTCTTC | 64 |
| 178 to 236) | TCTGGGAAGCTGTACCTCATTGGAATTGCAGCCATTGTGGTAGCTGTCATTATGATCTTTGAG | |
| | ATGATTCTGAGCATGGTGCTGTGCTGTGGCATCCGGAACAGCTCCGTGTACTGA | |
| CD81-IL- | MGVEGCTKCIKYLLFVFNFVFWLAGGVILGVALWLRHDPQTTSLLYLELGNKPAPNTFYVGIY | 95 |
| 12α | ILIAVGAVMMFVGFLGCYGAIQESQCLLGTFFTCLVILFACEVAAGIWGFVNKDQIAKDVKQF | |
| | YDQALQQAVMDDDANNAKAVVKTFHETLNCCGSNALTTLTTTILRNSLCPSGGGGSRVIPVSG | |
| | PARCLSQSRNLLKTTDDMVKTAREKLKHYSCTAEDIDHEDITRDQTSTLKTCLPLELHKNESC | |
| | LATRETSSTTRGSCLPPQKTSLMMTLCLGSIYEDLKMYQTEFQAINAALQNHNHQQIILDKGM | |
| | LVAIDELMQSLNHNGETLRQKPPVGEADPYRVKMKLCILLHAFSTRVVTINRVMGYLSSAGGG | |
| | GSGGNILTPLLQQDCIIQKIDELFSGKLYLIGIAAIVVAVIMIFEMILSMVLCCGIRNSSVY | |
| | ATGGGGGTGGAGGGCTGCACCAAATGCATCAAATACCTGCTCTTCGTCTTCAATTTCGTCTTC | |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCAGGAGGAGGAGGAAGCAGGGTCATTCCAGTCTCTGGA | |
| | CCTGCCAGGTGTCTTAGCCAGTCCCGAAACCTGCTGAAGACCACAGATGACATGGTGAAGACG | 96 |
| | GCCAGAGAAAAACTGAAACATTATTCCTGCACTGCTGAAGACATCGATCATGAAGACATCACA | |
| | CGGGACCAAACCAGCACATTGAAGACCTGTTTACCACTGGAACTACACAAGAACGAGAGTTGC | |
| | CTGGCTACTAGAGAGACTTCTTCCACAACAAGAGGGAGCTGCCTGCCCCCACAGAAGACGTCT | |
| | TTGATGATGACCCTGTGCCTTGGTAGCATCTATGAGGACTTGAAGATGTACCAGACAGAGTTC | |
| | CAGGCCATCAACGCAGCACTTCAGAATCACAACCATCAGCAGATCATTCTAGACAAGGGCATG | |
| | CTGGTGGCCATCGATGAGCTGATGCAGTCTCTGAATCATAATGGCGAGACTCTGCGCCAGAAA | |
| | CCTCCTGTGGGAGAAGCAGACCCTTACAGAGTGAAAATGAAGCTCTGCATCCTGCTTCACGCC | |
| | TTCAGCACCCGCGTCGTGACCATCAACAGGGTGATGGGCTATCTGAGCTCCGCCGGAGGAGGA | |
| | GGAAGCGGCGGCAACATACTCACCCCCTTACTGCAGCAAGATTGTCATCAGAAAATCGATGAG | |
| | CTCTTCTCTGGGAAGCTGTACCTCATTGGAATTGCAGCCATTGTGGTAGCTGTCATTATGATC | |
| | TTTGAGATGATTCTGAGCATGGTGCTGTGCTGTGGCATCCGGAACAGCTCCGTGTACTGA | |
| IL-12β | MCPQKLTISWFAIVLLVSPLMAMWELEKDVYVVEVDWTPDAPGETVNLTCDTPEEDDITWTSD | 97 |
| | QRHGVIGSGKTLTITVKEFLDAGQYTCHKGGETLSHSHLLLHKKENGIWSTEILKNFKNKTFL | |
| | KCEAPNYSGRFTCSWLVQRNMDLKFNIKSSSSSPDSRAVTCGMASLSAEKVTLDQRDYEKYSV | |
| | SCQEDVTCPTAEETLPIELALEARQQNKYENYSTSFFIRDIIKPDPPKNLQMKPLKNSQVEVS | |
| | WEYPDSWSTPHSYFSLKFFVRIQRKKEKMKETEEGCNQKGAFLVEKTSTEVQCKGGNVCVQAQ | |
| | DRYYNSSCSKWACVPCRVRS | |
| | ATGTGTCCTCAGAAGCTAACCATCTCCTGGTTTGCCATCGTTTTGCTGGTGTCTCCACTCATG | 98 |
| | GCCATGTGGGAGCTGGAGAAAGACGTTTATGTTGTAGAGGTGGACTGGACTCCCGATGCCCCT | |
| | GGAGAAACAGTGAACCTCACCTGTGACACGCCTGAAGAAGATGACATCACCTGGACCTCAGAC | |
| | CAGAGACATGGAGTCATAGGCTCTGGAAAGACCCTGACCATCACTGTCAAAGAGTTTCTAGAT | |
| | GCTGGCCAGTACACCTGCCACAAAGGAGGCGAGACTCTGAGCCACTCACATCTGCTGCTCCAC | |
| | AAGAAGGAAAATGGAATTTGGTCCACTGAAATTTTAAAAAATTTCAAAAACAAGACTTTCCTG | |
| | AAGTGTGAAGCACCAAATTACTCCGGACGGTTCACGTGCTCATGGCTGGTGCAAAGAAACATG | |
| | GACTTGAAGTTCAACATCAAGAGCAGTAGCAGTTCCCCTGACTCTCGGGCAGTGACATGTGGA | |
| | ATGGCGTCTCTGTCTGCAGAGAAGGTCACACTGGACCAAAGGGACTATGAGAAGTATTCAGTG | |
| | TCCTGCCAGGAGGATGTCACCTGCCCAACTGCCGAGGAGACCCTGCCCATTGAACTGGCGTTG | |
| | GAAGCACGGCAGCAGAATAAATATGAGAACTACAGCACCAGCTTCTTCATCAGGGACATCATC | |
| | AAACCAGACCCGCCCAAGAACTTGCAGATGAAGCCTTTGAAGAACTCACAGGTGGAGGTCAGC | |
| | TGGGAGTACCCTGACTCCTGGAGCACTCCCCATTCCTACTTCTCCCTCAAGTTCTTTGTTCGA | |
| | ATCCAGCGCAAGAAAGAAAAGATGAAGGAGACAGAGGAGGGGTGTAACCAGAAAGGTGCGTTC | |
| | CTCGTAGAGAAGACATCTACCGAAGTCCAATGCAAAGGCGGGAATGTCTGCGTGCAAGCTCAG | |
| | GATCGCTATTACAATTCCTCATGCAGCAAGTGGGCATGTGTTCCCTGCAGGGTCCGATCCTAG | |

TABLE 9

| | | SEQ |
|---|---|---|
| | Sequence | ID NO: |
| IL-6 | FPTSQVRRGDFTEDTTPNRPVYTTSQVGGLITHVLWEIVEMRKELCNGNSDCMNNDDALAENN | 99 |
| (from which | LKLPEIQRNDGCYQTGYNQEICLLKISSGLLEYHSYLEYMKNNLKDNKKDKARVLQRDTETLI | |
| signal peptide | HIFNQEVKDLHKIVLPTPISNALLTDKLESQKEWLRTKTIQFILKSLEEFLKVTLRSTRQT | |
| is removed) | TTCCCTACTTCACAAGTCCGGAGAGGAGACTTCACAGAGGATACCACTCCCAACAGACCTGTC | 100 |
| | TATACCACTTCACAAGTCGGAGGCTTAATTACACATGTTCTCTGGGAAATCGTGGAAATGAGA | |
| | AAAGAGTTGTGCAATGGCAATTCTGATTGTATGAACAACGATGATGCACTTGCAGAAAACAAT | |
| | CTGAAACTTCCAGAGATACAAAGAAATGATGGATGCTACCAAACTGGATATAATCAGGAAATT | |
| | TGCCTATTGAAAATTTCCTCTGGTCTTCTGGAGTACCATAGCTACCTGGAGTACATGAAGAAC | |
| | AACTTAAAAGATAACAAGAAAGACAAAGCCAGAGTCCTTCAGAGAGATACAGAAACTCTAATT | |
| | CATATCTTCAACCAAGAGGTAAAAGATTTACATAAAATAGTCCTTCCTACCCCAATTTCCAAT | |
| | GCTCTCCTAACAGATAAGCTGGAGTCACAGAAGGAGTGGCTAAGGACCAAGACCATCCAATTC | |
| | ATCTTGAAATCACTTGAAGAATTTCTAAAAGTCACTTTGAGATCTACTCGGCAAACC | |
| CD81 | MGVEGCTKCIKYLLFVFNFVFWLAGGVILGVALWLRHDPQTTSLLYLELGNKPAPNTFYVGIY | 61 |
| (amino acids | ILIAVGAVMMFVGFLGCYGAIQESQCLLGTFFTCLVILFACEVAAGIWGFVNKDQIAKDVKQF | |
| 1 to 177) | YDQALQQAVMDDDANNAKAVVKTFHETLNCCGSNALTTLTTTILRNSLCPS | |
| | ATGGGGGTGGAGGGCTGCACCAAATGCATCAAATACCTGCTCTTCGTCTTCAATTTCGTCTTC | 62 |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCA | |
| CD81 | GGNILTPLLQQDCHQKIDELFSGKLYLIGIAAIVVAVIMIFEMILSMVLCCGIRNSSVY | 63 |
| (amino acids | GGCGGCAACATACTCACCCCCTTACTGCAGCAAGATTGTCATCAGAAAATCGATGAGCTCTTC | 64 |
| 178 to 236) | TCTGGGAAGCTGTACCTCATTGGAATTGCAGCCATTGTGGTAGCTGTCATTATGATCTTTGAG | |
| | ATGATTCTGAGCATGGTGCTGTGCTGTGGCATCCGGAACAGCTCCGTGTACTGA | |
| CD81-IL-6 | MGVEGCTKCIKYLLFVFNFVFWLAGGVILGVALWLRHDPQTTSLLYLELGNKPAPNTFYVGIY | 101 |
| | ILIAVGAVMMFVGFLGCYGAIQESQCLLGTFFTCLVILFACEVAAGIWGFVNKDQIAKDVKQF | |
| | YDQALQQAVMDDDANNAKAVVKTFHETLNCCGSNALTTLTTTILRNSLCPSGGGGSFPTSQVR | |
| | RGDFTEDTTPNRPVYTTSQVGGLITHVLWEIVEMRKELCNGNSDCMNNDDALAENNLKLPEIQ | |
| | RNDGCYQTGYNQEICLLKISSGLLEYHSYLEYMKNNLKDNKKDKARVLQRDTETLIHIFNQEV | |
| | KDLHKIVLPTPISNALLTDKLESQKEWLRTKTIQFILKSLEEFLKVTLRSTRQTGGGGSGGNI | |
| | LTPLLQQDCHQKIDELFSGKLYLIGIAAIVVAVIMIFEMILSMVLCCGIRNSSVY | |
| | ATGGGGGTGGAGGGCTGCACCAAATGCATCAAATACCTGCTCTTCGTCTTCAATTTCGTCTTC | 102 |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCAGGAGGAGGAGGAAGCTTCCCTACTTCACAAGTCCGG | |
| | AGAGGAGACTTCACAGAGGATACCACTCCCAACAGACCTGTCTATACCACTTCACAAGTCGGA | |
| | GGCTTAATTACACATGTTCTCTGGGAAATCGTGGAAATGAGAAAAGAGTTGTGCAATGGCAAT | |
| | TCTGATTGTATGAACAACGATGATGCACTTGCAGAAAACAATCTGAAACTTCCAGAGATACAA | |
| | AGAAATGATGGATGCTACCAAACTGGATATAATCAGGAAATTTGCCTATTGAAAATTTCCTCT | |
| | GGTCTTCTGGAGTACCATAGCTACCTGGAGTACATGAAGAACAACTTAAAAGATAACAAGAAA | |
| | GACAAAGCCAGAGTCCTTCAGAGAGATACAGAAACTCTAATTCATATCTTCAACCAAGAGGTA | |
| | AAAGATTTACATAAAATAGTCCTTCCTACCCCAATTTCCAATGCTCTCCTAACAGATAAGCTG | |
| | GAGTCACAGAAGGAGTGGCTAAGGACCAAGACCATCCAATTCATCTTGAAATCACTTGAAGAA | |
| | TTTCTAAAAGTCACTTTGAGATCTACTCGGCAAACCGGAGGAGGAGGAAGCGGCGGCAACATA | |
| | CTCACCCCCTTACTGCAGCAAGATTGTCATCAGAAAATCGATGAGCTCTTCTCTGGGAAGCTG | |
| | TACCTCATTGGAATTGCAGCCATTGTGGTAGCTGTCATTATGATCTTTGAGATGATTCTGAGC | |
| | ATGGTGCTGTGCTGTGGCATCCGGAACAGCTCCGTGTACTGA | |

TABLE 10

| | | SEQ |
|---|---|---|
| | Sequence | ID NO: |
| hCD80 | MGHTRRQGTSPSKCPYLNFFQLLVLAGLSHFCSGVIHVTKEVKEVATLSCGHNVSVEELAQTR | 103 |
| | IYWQKEKKMVLTMMSGDMNIWPEYKNRTIFDITNNLSIVILALRPSDEGTYECVVLKYEKDAF | |
| | KREHLAEVTLSVKADFPTPSISDFEIPTSNIRRIICSTSGGFPEPHLSWLENGEELNAINTTV | |
| | SQDPETELYAVSSKLDFNMTTNHSFMCLIKYGHLRVNQTFNWNTTKQEHFPDNLLPSWAITLI | |
| | SVNGIFVICCLTYCFAPRCRERRRNERLRRESVRPV | |
| | ATGGGCCACACACGGAGGCAGGGAACATCACCATCCAAGTGTCCATACCTCAATTTCTTTCAG | |
| | CTCTTGGTGCTGGCTGGTCTTTCTCACTTCTGTTCAGGTGTTATCCACGTGACCAAGGAAGTG | 104 |
| | AAAGAAGTGGCAACGCTGTCCTGTGGTCACAATGTTTCTGTTGAAGAGCTGGCACAAACTCGC | |
| | ATCTACTGGCAAAAGGAGAAGAAAATGGTGCTGACTATGATGTCTGGGGACATGAATATATGG | |
| | CCCGAGTACAAGAACCGGACCATCTTTGATATCACTAATAACCTCTCCATTGTGATCCTGGCT | |
| | CTGCGCCCATCTGACGAGGGCACATACGAGTGTGTTGTTCTGAAGTATGAAAAAGACGCTTTC | |
| | AAGCGGGAACACCTGGCTGAAGTGACGTTATCAGTCAAAGCTGACTTCCCTACACCTAGTATA | |
| | TCTGACTTTGAAATTCCAACTTCTAATATTAGAAGGATAATTTGCTCAACCTCTGGAGGTTTT | |
| | CCAGAGCCTCACCTCTCCTGGTTGGAAAATGGAGAAGAATTAAATGCCATCAACACAACAGTT | |
| | TCCCAAGATCCTGAAACTGAGCTCTATGCTGTTAGCAGCAAACTGGATTTCAATATGACAACC | |
| | AACCACAGCTTCATGTGTCTCATCAAGTATGGACATTTAAGAGTGAATCAGACCTTCAACTGG | |
| | AATACAACCAAGCAAGAGCATTTTCCTGATAACCTGCTCCCATCCTGGGCCATTACCTTAATC | |
| | TCAGTAAATGGAATTTTTGTGATATGCTGCCTGACCTACTGCTTTGCCCCAAGATGCAGAGAG | |
| | AGAAGGAGGAATGAGAGATTGAGAAGGGAAAGTGTACGCCCTGTA | |
| hCD9 | MPVKGGTKCIKYLLFGFNFIFWLAGIAVLAIGLWLRFDSQTKSIFEQETNNNNSSFYTGVYIL | 105 |
| | IGAGALMMLVGFLGCCGAVQESQCMLGLFFGFLLVIFAIEIAAAIWGYSHKDEVIKEVQEFYK | |
| | DTYNKLKTKDEPQRETLKAIHYALNCCGLAGGVEQFISDICPKKDVLETFTVKSCPDAIKEVF | |
| | DNKFHIIGAVGIGIAVVMIFGMIFSMILCCAIRRNREMV | |
| | ATGCCGGTCAAAGGAGGCACCAAGTGCATCAAATACCTGCTGTTCGGATTTAACTTCATCTTC | 106 |
| | TGGCTTGCCGGGATTGCTGTCCTTGCCATTGGACTATGGCTCCGATTCGACTCTCAGACCAAG | |
| | AGCATCTTCGAGCAAGAAACTAATAATAATAATTCCAGCTTCTACACAGGAGTCTATATTCTG | |
| | ATCGGAGCCGGCGCCCTCATGATGCTGGTGGGCTTCCTGGGCTGCTGCGGGGCTGTGCAGGAG | |
| | TCCCAGTGCATGCTGGGACTGTTCTTCGGCTTCCTCTTGGTGATATTCGCCATTGAAATAGCT | |
| | GCGGCCATCTGGGGATATTCCCACAAGGATGAGGTGATTAAGGAAGTCCAGGAGTTTTACAAG | |
| | GACACCTACAACAAGCTGAAAACCAAGGATGAGCCCCAGCGGGAAACGCTGAAAGCCATCCAC | |
| | TATGCGTTGAACTGCTGTGGTTTGGCTGGGGGCGTGGAACAGTTTATCTCAGACATCTGCCCC | |
| | AAGAAGGACGTACTCGAAACCTTCACCGTGAAGTCCTGTCCTGATGCCATCAAAGAGGTCTTC | |
| | GACAATAAATTCCACATCATCGGCGCAGTGGGCATCGGCATTGCCGTGGTCATGATATTTGGC | |
| | ATGATCTTCAGTATGATCTTGTGCTGTGCTATCCGCAGGAACCGCGAGATGGTCTAG | |
| | MGHTRRQGTSPSKCPYLNFFQLLVLAGLSHFCSGVIHVTKEVKEVATLSCGHNVSVEELAQTR | 107 |
| | IYWQKEKKMVLTMMSGDMNIWPEYKNRTIFDITNNLSIVILALRPSDEGTYECVVLKYEKDAF | |
| | KREHLAEVTLSVKADFPTPSISDFEIPTSNIRRIICSTSGGFPEPHLSWLENGEELNAINTTV | |
| | SQDPETELYAVSSKLDFNMTTNHSFMCLIKYGHLRVNQTFNWNTTKQEHFPDNLLPSWAITLI | |
| | SVNGIFVICCLTYCFAPRCRERRRNERLRRESVRPVMPVKGGTKCIKYLLFGFNFIFWLAGIA | |
| | VLAIGLWLRFDSQTKSIFEQETNNNNSSFYTGVYILIGAGALMMLVGFLGCCGAVQESQCMLG | |
| | LFFGFLLVIFAIEIAAAIWGYSHKDEVIKEVQEFYKDTYNKLKTKDEPQRETLKATHYALNCC | |
| | GLAGGVEQFISDICPKKDVLETFTVKSCPDAIKEVFDNKFHIIGAVGIGIAVVMIFGMIFSMI | |
| | LCCAIRRNREMV | |
| hCD80- | ATGGGCCACACACGGAGGCAGGGAACATCACCATCCAAGTGTCCATACCTCAATTTCTTTCAG | 108 |
| hCD9 | CTCTTGGTGCTGGCTGGTCTTTCTCACTTCTGTTCAGGTGTTATCCACGTGACCAAGGAAGTG | |
| | AAAGAAGTGGCAACGCTGTCCTGTGGTCACAATGTTTCTGTTGAAGAGCTGGCACAAACTCGC | |
| | ATCTACTGGCAAAAGGAGAAGAAAATGGTGCTGACTATGATGTCTGGGGACATGAATATATGG | |
| | CCCGAGTACAAGAACCGGACCATCTTTGATATCACTAATAACCTCTCCATTGTGATCCTGGCT | |
| | CTGCGCCCATCTGACGAGGGCACATACGAGTGTGTTGTTCTGAAGTATGAAAAAGACGCTTTC | |
| | AAGCGGGAACACCTGGCTGAAGTGACGTTATCAGTCAAAGCTGACTTCCCTACACCTAGTATA | |
| | TCTGACTTTGAAATTCCAACTTCTAATATTAGAAGGATAATTTGCTCAACCTCTGGAGGTTTT | |
| | CCAGAGCCTCACCTCTCCTGGTTGGAAAATGGAGAAGAATTAAATGCCATCAACACAACAGTT | |
| | TCCCAAGATCCTGAAACTGAGCTCTATGCTGTTAGCAGCAAACTGGATTTCAATATGACAACC | |
| | AACCACAGCTTCATGTGTCTCATCAAGTATGGACATTTAAGAGTGAATCAGACCTTCAACTGG | |
| | AATACAACCAAGCAAGAGCATTTTCCTGATAACCTGCTCCCATCCTGGGCCATTACCTTAATC | |
| | TCAGTAAATGGAATTTTTGTGATATGCTGCCTGACCTACTGCTTTGCCCCAAGATGCAGAGAG | |
| | AGAAGGAGGAATGAGAGATTGAGAAGGGAAAGTGTACGCCCTGTAATGCCGGTCAAAGGAGGC | |
| | ACCAAGTGCATCAAATACCTGCTGTTCGGATTTAACTTCATCTTCTGGCTTGCCGGGATTGCT | |
| | GTCCTTGCCATTGGACTATGGCTCCGATTCGACTCTCAGACCAAGAGCATCTTCGAGCAAGAA | |
| | ACTAATAATAATAATTCCAGCTTCTACACAGGAGTCTATATTCTGATCGGAGCCGGCGCCCTC | |
| | ATGATGCTGGTGGGCTTCCTGGGCTGCTGCGGGGCTGTGCAGGAGTCCCAGTGCATGCTGGGA | |
| | CTGTTCTTCGGCTTCCTCTTGGTGATATTCGCCATTGAAATAGCTGCGGCCATCTGGGGATAT | |
| | TCCCACAAGGATGAGGTGATTAAGGAAGTCCAGGAGTTTTACAAGGACACCTACAACAAGCTG | |
| | AAAACCAAGGATGAGCCCCAGCGGGAAACGCTGAAAGCCATCCACTATGCGTTGAACTGCTGT | |
| | GGTTTGGCTGGGGGCGTGGAACAGTTTATCTCAGACATCTGCCCCAAGAAGGACGTACTCGAA | |
| | ACCTTCACCGTGAAGTCCTGTCCTGATGCCATCAAAGAGGTCTTCGACAATAAATTCCACATC | |
| | ATCGGCGCAGTGGGCATCGGCATTGCCGTGGTCATGATATTTGGCATGATCTTCAGTATGATC | |
| | TTGTGCTGTGCTATCCGCAGGAACCGCGAGATGGTCTAG | |

TABLE 11

| | | SEQ |
|---|---|---|
| | Sequence | ID NO: |
| hIL-2 | APTSSSTKKTQLQLEHLLLDLQMILNGINNYKNPKLTRMLTFKFYMPKKATELKHLQCLEEEL | 109 |
| (from which | KPLEEVLNLAQSKNFHLRPRDLISNINVIVLELKGSETTFMCEYADETATIVEFLNRWITFCQ | |
| signal peptide | SIISTLT | |
| is removed) | GCACCTACTTCAAGTTCTACAAAGAAAACACAGCTACAACTGGAGCATTTACTGCTGGATTTA | 110 |
| | CAGATGATTTTGAATGGAATTAATAATTACAAGAATCCCAAACTCACCAGGATGCTCACATTT | |
| | AAGTTTTACATGCCCAAGAAGGCCACAGAACTGAAACATCTTCAGTGTCTAGAAGAAGAACTC | |
| | AAACCTCTGGAGGAAGTGCTAAATTTAGCTCAAAGCAAAAACTTTCACTTAAGACCCAGGGAC | |
| | TTAATCAGCAATATCAACGTAATAGTTCTGGAACTAAAGGGATCTGAAACAACATTCATGTGT | |
| | GAATATGCTGATGAGACAGCAACCATTGTAGAATTTCTGAACAGATGGATTACCTTTTGTCAA | |
| | AGCATCATCTCAACACTGACT | |
| hCD63 | MAVEGGMKCVKFLLYVLLLAFCACAVGLIAVGVGAQLVLSQTIIQGATPGSLLPVVIIAVGVF | 111 |
| (amino acids | LFLVAFVGCCGACKENYCLMITFAIFLSLIMLVEVAAAIAGYVFRDKVMSEFNNNFRQQMENY | |
| 1 to 170) | PKNNHTASILDRMQADFKCCGAANYTDWEKIPSMSKNRVPDSCC | |
| | ATGGCGGTGGAAGGAGGAATGAAATGTGTGAAGTTCTTGCTCTACGTCCTCCTGCTGGCCTTT | 112 |
| | TGCGCCTGTGCAGTGGGACTGATTGCCGTGGGTGTCGGGGCACAGCTTGTCCTGAGTCAGACC | |
| | ATAATCCAGGGGGCTACCCCTGGCTCTCTGTTGCCAGTGGTCATCATCGCAGTGGGTGTCTTC | |
| | CTCTTCCTGGTGGCTTTTGTGGGCTGCTGCGGGGCCTGCAAGGAGAACTATTGTCTTATGATC | |
| | ACGTTTGCCATCTTTCTGTCTCTTATCATGTTGGTGGAGGTGGCCGCAGCCATTGCTGGCTAT | |
| | GTGTTTAGAGATAAGGTGATGTCAGAGTTTAATAACAACTTCCGGCAGCAGATGGAGAATTAC | |
| | CCGAAAAACAACCACACTGCTTCGATCCTGGACAGGATGCAGGCAGATTTTAAGTGCTGTGGG | |
| | GCTGCTAACTACACAGATTGGGAGAAAATCCCTTCCATGTCGAAGAACCGAGTCCCCGACTCC | |
| | TGCTGC | |
| hCD63 | INVTVGCGINFNEKAIHKEGCVEKIGGWLRKNVLVVAAAALGIAFVEVLGIVFACCLVKSIRS | 113 |
| (amino acids | GYEVM | |
| 171 to 238) | ATTAATGTTACTGTGGGCTGTGGGATTAATTTCAACGAGAAGGCGATCCATAAGGAGGGCTGT | 114 |
| | GTGGAGAAGATTGGGGGCTGGCTGAGGAAAAATGTGCTGGTGGTAGCTGCAGCAGCCCTTGGA | |
| | ATTGCTTTTGTCGAGGTTTTGGGAATTGTCTTTGCCTGCTGCCTCGTGAAGAGTATCAGAAGT | |
| | GGCTACGAGGTGATGTAG | |
| hCD63-IL-2 | MAVEGGMKCVKELLYVLLLAFCACAVGLIAVGVGAQLVLSQTIIQGATPGSLLPVVIIAVGVF | 115 |
| | LFLVAFVGCCGACKENYCLMITFAIFLSLIMLVEVAAAIAGYVFRDKVMSEFNNNFRQQMENY | |
| | PKNNHTASILDRMQADFKCCGAANYTDWEKIPSMSKNRVPDSCCGGGGSAPTSSSTKKTQLQL | |
| | EHLLLDLQMILNGINNYKNPKLTRMLTFKFYMPKKATELKHLQCLEEELKPLEEVLNLAQSKN | |
| | FHLRPRDLISNINVIVLELKGSETTFMCEYADETATIVEFLNRWITFCQSIISTLTGGGGSIN | |
| | VTVGCGINFNEKAIHKEGCVEKIGGWLRKNVLVVAAAALGIAFVEVLGIVFACCLVKSIRSGY | |
| | EVM | |
| | ATGGCGGTGGAAGGAGGAATGAAATGTGTGAAGTTCTTGCTCTACGTCCTCCTGCTGGCCTTT | 116 |
| | TGCGCCTGTGCAGTGGGACTGATTGCCGTGGGTGTCGGGGCACAGCTTGTCCTGAGTCAGACC | |
| | ATAATCCAGGGGGCTACCCCTGGCTCTCTGTTGCCAGTGGTCATCATCGCAGTGGGTGTCTTC | |
| | CTCTTCCTGGTGGCTTTTGTGGGCTGCTGCGGGGCCTGCAAGGAGAACTATTGTCTTATGATC | |
| | ACGTTTGCCATCTTTCTGTCTCTTATCATGTTGGTGGAGGTGGCCGCAGCCATTGCTGGCTAT | |
| | GTGTTTAGAGATAAGGTGATGTCAGAGTTTAATAACAACTTCCGGCAGCAGATGGAGAATTAC | |
| | CCGAAAAACAACCACACTGCTTCGATCCTGGACAGGATGCAGGCAGATTTTAAGTGCTGTGGG | |
| | GCTGCTAACTACACAGATTGGGAGAAAATCCCTTCCATGTCGAAGAACCGAGTCCCCGACTCC | |
| | TGCTGCGGAGGAGGAGGAAGCGCACCTACTTCAAGTTCTACAAAGAAAACACAGCTACAACTG | |
| | GAGCATTTACTGCTGGATTTACAGATGATTTTGAATGGAATTAATAATTACAAGAATCCCAAA | |
| | CTCACCAGGATGCTCACATTTAAGTTTTACATGCCCAAGAAGGCCACAGAACTGAAACATCTT | |
| | CAGTGTCTAGAAGAAGAACTCAAACCTCTGGAGGAAGTGCTAAATTTAGCTCAAAGCAAAAAC | |
| | TTTCACTTAAGACCCAGGGACTTAATCAGCAATATCAACGTAATAGTTCTGGAACTAAAGGGA | |
| | TCTGAAACAACATTCATGTGTGAATATGCTGATGAGACAGCAACCATTGTAGAATTTCTGAAC | |
| | AGATGGATTACCTTTTGTCAAAGCATCATCTCAACACTGACTGGAGGAGGAGGAAGCATTAAT | |
| | GTTACTGTGGGCTGTGGGATTAATTTCAACGAGAAGGCGATCCATAAGGAGGGCTGTGTGGAG | |
| | AAGATTGGGGGCTGGCTGAGGAAAAATGTGCTGGTGGTAGCTGCAGCAGCCCTTGGAATTGCT | |
| | TTTGTCGAGGTTTTGGGAATTGTCTTTGCCTGCTGCCTCGTGAAGAGTATCAGAAGTGGCTAC | |
| | GAGGTGATGTAG | |

TABLE 12

| | | SEQ |
|---|---|---|
| | Sequence | ID NO: |
| Signal peptide | MSRSVALAVLALLSLSGLEA | 117 |
| of hβ₂ | ATGTCTCGCTCCGTGGCCTTAGCTGTGCTCGCGCTACTCTCTCTTTCTGGCCTGGAGGCT | 118 |
| microglobulin | | |
| WT1 peptide | CYTWNQMNL | 119 |
| (for MHC class | TGCTACACCTGGAACCAGATGAACCTG | 120 |
| I molecule) | | |
| Peptide linker 1 | GGGASGGGGGGGGS | 5 |
| | GGCGGAGGTGCCTCTGGCGGTGGGGGCAGCGGTGGAGGGGGCAGT | 6 |
| hβ₂ | IQRTPKIQVYSRHPAENGKSNFLNCYVSGFHPSDIEVDLLKNGERIEKVEHSDLSFSKDWSFY | 121 |
| Microglobulin | LLYYTEFTPTEKDEYACRVNHVTLSQPKIVKWDRDM | |
| (from which | ATCCAGCGTACTCCAAAGATTCAGGTTTACTCACGTCATCCAGCAGAGAATGGAAAGTCAAAT | 122 |
| signal peptide | TTCCTGAATTGCTATGTGTCTGGGTTTCATCCATCCGACATTGAAGTTGACTTACTGAAGAAT | |
| is removed) | GGAGAGAGAATTGAAAAAGTGGAGCATTCAGACTTGTCTTTCAGCAAGGACTGGTCTTTCTAT | |
| | CTCTTGTACTACACTGAATTCACCCCCACTGAAAAAGATGAGTATGCCTGCCGTGTGAACCAT | |
| | GTGACTTTGTCACAGCCCAAGATAGTTAAGTGGGATCGAGACATG | |
| hMHC class | GSHSMRYFSTSVSRPGRGEPRFIAVGYVDDTQFVRFDSDAASQRMEPRAPWIEQEGPEYWDEE | 123 |
| Iα chain | TGKVKAHSQTDRENLRIALRYYNQSEAGSHTLQMMFGCDVGSDGRFLRGYHQYAYDGKDYIAL | |
| (from which | KEDLRSWTAADMAAQITKRKWEAAHVAEQQRAYLEGTCVDGLRRYLENGKETLQRTDPPKTHM | |
| signal peptide | THHPISDHEATLRCWALGFYPAEITLTWQRDGEDQTQDTELVETRPAGDGTFQKWAAVVVPSG | |
| is removed) | EEQRYTCHVQHEGLPKPLTLRWEPSSQPTVPIVGIIAGLVLLGAVITGAVVAAVMWRRNSSDR | |
| | KGGSYSQAASSDSAQGSDVSLTACKV | |
| | GGCTCCCACTCCATGAGGTATTTCTCCACATCCGTGTCCCGGCCCGGCCGCGGGGAGCCCCGC | 124 |
| | TTCATCGCCGTGGGCTACGTGGACGACACGCAGTTCGTGCGGTTCGACAGCGACGCCGCGAGC | |
| | CAGAGGATGGAGCCGCGGGCGCCGTGGATAGAGCAGGAGGGGCCGGAGTATTGGGACGAGGAG | |
| | ACAGGGAAAGTGAAGGCCCACTCACAGACTGACCGAGAGAACCTGCGGATCGCGCTCCGCTAC | |
| | TACAACCAGAGCGAGGCCGGTTCTCACACCCTCCAGATGATGTTTGGCTGCGACGTGGGGTCG | |
| | GACGGGCGCTTCCTCCGCGGGTACCACCAGTACGCCTACGACGGCAAGGATTACATCGCCCTG | |
| | AAAGAGGACCTGCGCTCTTGGACCGCGGCGGACATGGCGGCTCAGATCACCAAGCGCAAGTGG | |
| | GAGGCGGCCCATGTGGCGGAGCAGCAGAGAGCCTACCTGGAGGGCACGTGCGTGGACGGGCTC | |
| | CGCAGATACCTGGAGAACGGGAAGGAGACGCTGCAGCGCACGGACCCCCCCAAGACACATATG | |
| | ACCCACCACCCCATCTCTGACCATGAGGCCACTCTGAGATGCTGGGCCCTGGGCTTCTACCCT | |
| | GCGGAGATCACACTGACCTGGCAGCGGGATGGGGAGGACCAGACCCAGGACACGGAGCTTGTG | |
| | GAGACCAGGCCTGCAGGGGATGGAACCTTCCAGAAGTGGGCAGCTGTGGTGGTACCTTCTGGA | |
| | GAGGAGCAGAGATACACCTGCCATGTGCAGCATGAGGGTCTGCCCAAGCCCCTCACCCTGAGA | |
| | TGGGAGCCATCTTCCCAGCCCACCGTCCCCATCGTGGGCATCATTGCTGGCCTGGTTCTCCTT | |
| | GGAGCTGTGATCACTGGAGCTGTGGTCGCTGCTGTGATGTGGAGGAGGAACAGCTCAGATAGA | |
| | AAAGGAGGGAGCTACTCTCAGGCTGCAAGCAGTGACAGTGCCCAGGGCTCTGATGTGTCTCTC | |
| | ACAGCTTGTAAAGTG | |
| Peptide linker 2 | GGGGSGGGGSGGGGSGGGGS | 11 |
| | GGGGGGGGAGGCTCCGGTGGAGGGGGGTCTGGAGGGGGGGGGTCTGGTGGAGGCGGAAGT | 12 |
| h Single chain | IQRTPKIQVYSRHPAENGKSNFLNCYVSGFHPSDIEVDLLKNGERIEKVEHSDLSFSKDWSFY | 125 |
| MHC class | LLYYTEFTPTEKDEYACRVNHVTLSQPKIVKWDRDMGGGGSGGGGGGGGSGGGGSGSHSMRY | |
| I molecule | FSTSVSRPGRGEPRFIAVGYVDDTQFVRFDSDAASQRMEPRAPWIEQEGPEYWDEETGKVKAH | |
| (β₂ | SQTDRENLRIALRYYNQSEAGSHTLQMMFGCDVGSDGRFLRGYHQYAYDGKDYIALKEDLRSW | |
| microglobulin | TAADMAAQITKRKWEAAHVAEQQRAYLEGTCVDGLRRYLENGKETLQRTDPPKTHMTHHPISD | |
| (from which | HEATLRCWALGFYPAEITLTWQRDGEDQTQDTELVETRPAGDGTFQKWAAVVVPSGEEQRYTC | |
| signal peptide | HVQHEGLPKPLTLRWEPSSQPTVPIVGIIAGLVLLGAVITGAVVAAVMWRRNSSDRKGGSYSQ | |
| is removed) + | AASSDSAQGSDVSLTACKV | |
| peptide linker | ATCCAGCGTACTCCAAAGATTCAGGTTTACTCACGTCATCCAGCAGAGAATGGAAAGTCAAAT | 126 |
| 2 + MHC class | TTCCTGAATTGCTATGTGTCTGGGTTTCATCCATCCGACATTGAAGTTGACTTACTGAAGAAT | |
| Iα chain | GGAGAGAGAATTGAAAAAGTGGAGCATTCAGACTTGTCTTTCAGCAAGGACTGGTCTTTCTAT | |
| (from which | CTCTTGTACTACACTGAATTCACCCCCACTGAAAAAGATGAGTATGCCTGCCGTGTGAACCAT | |
| signal peptide | GTGACTTTGTCACAGCCCAAGATAGTTAAGTGGGATCGAGACATGGGGGGGGGAGGCTCCGGT | |
| is removed)) | GGAGGGGGGTCTGGAGGGGGGGGGTCTGGTGGAGGCGGAAGTGGCTCCCACTCCATGAGGTAT | |
| | TTCTCCACATCCGTGTCCCGGCCCGGCCGCGGGGAGCCCCGCTTCATCGCCGTGGGCTACGTG | |
| | GACGACACGCAGTTCGTGCGGTTCGACAGCGACGCCGCGAGCCAGAGGATGGAGCCGCGGGCG | |
| | CCGTGGATAGAGCAGGAGGGGCCGGAGTATTGGGACGAGGAGACAGGGAAAGTGAAGGCCCAC | |
| | TCACAGACTGACCGAGAGAACCTGCGGATCGCGCTCCGCTACTACAACCAGAGCGAGGCCGGT | |
| | TCTCACACCCTCCAGATGATGTTTGGCTGCGACGTGGGGTCGGACGGGCGCTTCCTCCGCGGG | |
| | TACCACCAGTACGCCTACGACGGCAAGGATTACATCGCCCTGAAAGAGGACCTGCGCTCTTGG | |
| | ACCGCGGCGGACATGGCGGCTCAGATCACCAAGCGCAAGTGGGAGGCGGCCCATGTGGCGGAG | |
| | CAGCAGAGAGCCTACCTGGAGGGCACGTGCGTGGACGGGCTCCGCAGATACCTGGAGAACGGG | |
| | AAGGAGACGCTGCAGCGCACGGACCCCCCCAAGACACATATGACCCACCACCCCATCTCTGAC | |
| | CATGAGGCCACTCTGAGATGCTGGGCCCTGGGCTTCTACCCTGCGGAGATCACACTGACCTGG | |
| | CAGCGGGATGGGGAGGACCAGACCCAGGACACGGAGCTTGTGGAGACCAGGCCTGCAGGGGAT | |
| | GGAACCTTCCAGAAGTGGGCAGCTGTGGTGGTACCTTCTGGAGAGGAGCAGAGATACACCTGC | |
| | CATGTGCAGCATGAGGGTCTGCCCAAGCCCCTCACCCTGAGATGGGAGCCATCTTCCCAGCCC | |
| | ACCGTCCCCATCGTGGGCATCATTGCTGGCCTGGTTCTCCTTGGAGCTGTGATCACTGGAGCT | |
| | GTGGTCGCTGCTGTGATGTGGAGGAGGAACAGCTCAGATAGAAAAGGAGGGAGCTACTCTCAG | |
| | GCTGCAAGCAGTGACAGTGCCCAGGGCTCTGATGTGTCTCTCACAGCTTGTAAAGTG | |
| hsc-Trimer | MSRSVALAVLALLSLSGLEACYTWNQMNLGGGASGGGGSGGGGSIQRTPKIQVYSRHPAENGK | 127 |
| (WT1 peptide 1 + | SNFLNCYVSGFHPSDIEVDLLKNGERIEKVEHSDLSFSKDWSFYLLYYTEFTPTEKDEYACRV | |
| peptide linker | NHVTLSQPKIVKWDRDMGGGGSGGGGSGGGGSGGGGSGSHSMRYFSTSVSRPGRGEPRFIAVG | |
| 1 + single chain | YVDDTQFVRFDSDAASQRMEPRAPWIEQEGPEYWDEETGKVKAHSQTDRENLRIALRYYNQSE | |
| MHC class | AGSHTLQMMFGCDVGSDGRFLRGYHQYAYDGKDYIALKEDLRSWTAADMAAQITKRKWEAAHV | |
| I molecule) | AEQQRAYLEGTCVDGLRRYLENGKETLQRTDPPKTHMTHHPISDHEATLRCWALGFYPAEITL | |
| | TWQRDGEDQTQDTELVETRPAGDGTFQKWAAVVVPSGEEQRYTCHVQHEGLPKPLTLRWEPSS | |
| | QPTVPIVGIIAGLVLLGAVITGAVVAAVMWRRNSSDRKGGSYSQAASSDSAQGSDVSLTACKV | |
| | ATGTCTCGCTCCGTGGCCTTAGCTGTGCTCGCGCTACTCTCTCTTTCTGGCCTGGAGGCTTGC | 128 |
| | TACACCTGGAACCAGATGAACCTGGGCGGAGGTGCCTCTGGCGGTGGGGGCAGCGGTGGAGGG | |
| | GGCAGTATCCAGCGTACTCCAAAGATTCAGGTTTACTCACGTCATCCAGCAGAGAATGGAAAG | |
| | TCAAATTTCCTGAATTGCTATGTGTCTGGGTTTCATCCATCCGACATTGAAGTTGACTTACTG | |
| | AAGAATGGAGAGAGAATTGAAAAAGTGGAGCATTCAGACTTGTCTTTCAGCAAGGACTGGTCT | |
| | TTCTATCTCTTGTACTACACTGAATTCACCCCCACTGAAAAAGATGAGTATGCCTGCCGTGTG | |
| | AACCATGTGACTTTGTCACAGCCCAAGATAGTTAAGTGGGATCGAGACATGGGGGGGGGAGGC | |
| | TCCGGTGGAGGGGGGTCTGGAGGGGGGGGGTCTGGTGGAGGCGGAAGTGGCTCCCACTCCATG | |
| | AGGTATTTCTCCACATCCGTGTCCCGGCCCGGCCGCGGGGAGCCCCGCTTCATCGCCGTGGGC | |
| | TACGTGGACGACACGCAGTTCGTGCGGTTCGACAGCGACGCCGCGAGCCAGAGGATGGAGCCG | |
| | CGGGCGCCGTGGATAGAGCAGGAGGGGCCGGAGTATTGGGACGAGGAGACAGGGAAAGTGAAG | |
| | GCCCACTCACAGACTGACCGAGAGAACCTGCGGATCGCGCTCCGCTACTACAACCAGAGCGAG | |
| | GCCGGTTCTCACACCCTCCAGATGATGTTTGGCTGCGACGTGGGGTCGGACGGGCGCTTCCTC | |
| | CGCGGGTACCACCAGTACGCCTACGACGGCAAGGATTACATCGCCCTGAAAGAGGACCTGCGC | |
| | TCTTGGACCGCGGCGGACATGGCGGCTCAGATCACCAAGCGCAAGTGGGAGGCGGCCCATGTG | |
| | GCGGAGCAGCAGAGAGCCTACCTGGAGGGCACGTGCGTGGACGGGCTCCGCAGATACCTGGAG | |
| | AACGGGAAGGAGACGCTGCAGCGCACGGACCCCCCCAAGACACATATGACCCACCACCCCATC | |
| | TCTGACCATGAGGCCACTCTGAGATGCTGGGCCCTGGGCTTCTACCCTGCGGAGATCACACTG | |
| | ACCTGGCAGCGGGATGGGGAGGACCAGACCCAGGACACGGAGCTTGTGGAGACCAGGCCTGCA | |
| | GGGGATGGAACCTTCCAGAAGTGGGCAGCTGTGGTGGTACCTTCTGGAGAGGAGCAGAGATAC | |
| | ACCTGCCATGTGCAGCATGAGGGTCTGCCCAAGCCCCTCACCCTGAGATGGGAGCCATCTTCC | |
| | CAGCCCACCGTCCCCATCGTGGGCATCATTGCTGGCCTGGTTCTCCTTGGAGCTGTGATCACT | |
| | GGAGCTGTGGTCGCTGCTGTGATGTGGAGGAGGAACAGCTCAGATAGAAAAGGAGGGAGCTAC | |
| | TCTCAGGCTGCAAGCAGTGACAGTGCCCAGGGCTCTGATGTGTCTCTCACAGCTTGTAAAGTG | |
| hCD81 | MGVEGCTKCIKYLLFVFNFVFWLAGGVILGVALWLRHDPQTTNLLYLELGDKPAPNTFYVGIY | 129 |
| | ILIAVGAVMMFVGFLGCYGAIQESQCLLGTFFTCLVILFACEVAAGIWGFVNKDQIAKDVKQF | |
| | YDQALQQAVVDDDANNAKAVVKTFHETLDCCGSSTLTALTTSVLKNNLCPSGSNIISNLFKED | |
| | CHQKIDDLFSGKLYLIGIAAIVVAVIMIFEMILSMVLCCGIRNSSVY | |
| | ATGGGAGTGGAGGGCTGCACCAAGTGCATCAAGTACCTGCTCTTCGTCTTCAATTTCGTCTTC | 130 |
| | TGGCTGGCTGGAGGCGTGATCCTGGGTGTGGCCCTGTGGCTCCGCCATGACCCGCAGACCACC | |
| | AACCTCCTGTATCTGGAGCTGGGAGACAAGCCCGCGCCCAACACCTTCTATGTAGGCATCTAC | |
| | ATCCTCATCGCTGTGGGCGCTGTCATGATGTTCGTTGGCTTCCTGGGCTGCTACGGGGCCATC | |
| | CAGGAATCCCAGTGCCTGCTGGGGACGTTCTTCACCTGCCTGGTCATCCTGTTTGCCTGTGAG | |
| | GTGGCCGCCGGCATCTGGGGCTTTGTCAACAAGGACCAGATCGCCAAGGATGTGAAGCAGTTC | |
| | TATGACCAGGCCCTACAGCAGGCCGTGGTGGATGATGACGCCAACAACGCCAAGGCTGTGGTG | |
| | AAGACCTTCCACGAGACGCTTGACTGCTGTGGCTCCAGCACACTGACTGCTTTGACCACCTCA | |
| | GTGCTCAAGAACAATTTGTGTCCCTCGGGCAGCAACATCATCAGCAACCTCTTCAAGGAGGAC | |
| | TGCCACCAGAAGATCGATGACCTCTTCTCCGGGAAGCTGTACCTCATCGGCATTGCTGCCATC | |
| | GTGGTCGCTGTGATCATGATCTTCGAGATGATCCTGAGCATGGTGCTGTGCTGTGGCATCCGG | |
| | AACAGCTCCGTGTACTGA | |
| hsc-Trimer- | MSRSVALAVLALLSLSGLEACYTWNQMNLGGGASGGGGSGGGGSIQRTPKIQVYSRHPAENGK | 131 |
| CD81 | SNFLNCYVSGFHPSDIEVDLLKNGERIEKVEHSDLSFSKDWSFYLLYYTEFTPTEKDEYACRV | |
| (sc-Trimer + | NHVTLSQPKIVKWDRDMGGGGSGGGGSGGGGSGGGGSGSHSMRYFSTSVSRPGRGEPRFIAVG | |
| CD81) | YVDDTQFVRFDSDAASQRMEPRAPWIEQEGPEYWDEETGKVKAHSQTDRENLRIALRYYNQSE | |
| | AGSHTLQMMFGCDVGSDGRFLRGYHQYAYDGKDYTALKEDLRSWTAADMAAQTTKRKWEAAHV | |
| | AEQQRAYLEGTCVDGLRRYLENGKETLQRTDPPKTHMTHHPISDHEATLRCWALGFYPAEITL | |
| | TWQRDGEDQTQDTELVETRPAGDGTFQKWAAVVVPSGEEQRYTCHVQHEGLPKPLTLRWEPSS | |
| | QPTVPIVGIIAGLVLLGAVITGAVVAAVMWRRNSSDRKGGSYSQAASSDSAQGSDVSLTACKV | |
| | MGVEGCTKCIKYLLFVFNFVFWLAGGVILGVALWLRHDPQTTNLLYLELGDKPAPNTFYVGIY | |
| | ILIAVGAVMMFVGFLGCYGAIQESQCLLGTFFTCLVILFACEVAAGIWGFVNKDQIAKDVKQF | |
| | YDQALQQAVVDDDANNAKAVVKTFHETLDCCGSSTLTALTTSVLKNNLCPSGSNIISNLFKED | |
| | CHQKIDDLFSGKLYLIGIAAIVVAVIMIFEMILSMVLCCGIRNSSVY | |
| | ATGTCTCGCTCCGTGGCCTTAGCTGTGCTCGCGCTACTCTCTCTTTCTGGCCTGGAGGCTTGC | 132 |
| | TACACCTGGAACCAGATGAACCTGGGCGGAGGTGCCTCTGGCGGTGGGGGCAGCGGTGGAGGG | |
| | GGCAGTATCCAGCGTACTCCAAAGATTCAGGTTTACTCACGTCATCCAGCAGAGAATGGAAAG | |
| | TCAAATTTCCTGAATTGCTATGTGTCTGGGTTTCATCCATCCGACATTGAAGTTGACTTACTG | |
| | AAGAATGGAGAGAGAATTGAAAAAGTGGAGCATTCAGACTTGTCTTTCAGCAAGGACTGGTCT | |
| | TTCTATCTCTTGTACTACACTGAATTCACCCCCACTGAAAAAGATGAGTATGCCTGCCGTGTG | |
| | AACCATGTGACTTTGTCACAGCCCAAGATAGTTAAGTGGGATCGAGACATGGGGGGGGGAGGC | |
| | TCCGGTGGAGGGGGGTCTGGAGGGGGGGGGTCTGGTGGAGGCGGAAGTGGCTCCCACTCCATG | |
| | AGGTATTTCTCCACATCCGTGTCCCGGCCCGGCCGCGGGGAGCCCCGCTTCATCGCCGTGGGC | |
| | TACGTGGACGACACGCAGTTCGTGCGGTTCGACAGCGACGCCGCGAGCCAGAGGATGGAGCCG | |
| | CGGGCGCCGTGGATAGAGCAGGAGGGGCCGGAGTATTGGGACGAGGAGACAGGGAAAGTGAAG | |
| | GCCCACTCACAGACTGACCGAGAGAACCTGCGGATCGCGCTCCGCTACTACAACCAGAGCGAG | |
| | GCCGGTTCTCACACCCTCCAGATGATGTTTGGCTGCGACGTGGGGTCGGACGGGCGCTTCCTC | |
| | CGCGGGTACCACCAGTACGCCTACGACGGCAAGGATTACATCGCCCTGAAAGAGGACCTGCGC | |
| | TCTTGGACCGCGGCGGACATGGCGGCTCAGATCACCAAGCGCAAGTGGGAGGCGGCCCATGTG | |
| | GCGGAGCAGCAGAGAGCCTACCTGGAGGGCACGTGCGTGGACGGGCTCCGCAGATACCTGGAG | |
| | AACGGGAAGGAGACGCTGCAGCGCACGGACCCCCCCAAGACACATATGACCCACCACCCCATC | |
| | TCTGACCATGAGGCCACTCTGAGATGCTGGGCCCTGGGCTTCTACCCTGCGGAGATCACACTG | |
| | ACCTGGCAGCGGGATGGGGAGGACCAGACCCAGGACACGGAGCTTGTGGAGACCAGGCCTGCA | |
| | GGGGATGGAACCTTCCAGAAGTGGGCAGCTGTGGTGGTACCTTCTGGAGAGGAGCAGAGATAC | |
| | ACCTGCCATGTGCAGCATGAGGGTCTGCCCAAGCCCCTCACCCTGAGATGGGAGCCATCTTCC | |
| | CAGCCCACCGTCCCCATCGTGGGCATCATTGCTGGCCTGGTTCTCCTTGGAGCTGTGATCACT | |
| | GGAGCTGTGGTCGCTGCTGTGATGTGGAGGAGGAACAGCTCAGATAGAAAAGGAGGGAGCTAC | |
| | TCTCAGGCTGCAAGCAGTGACAGTGCCCAGGGCTCTGATGTGTCTCTCACAGCTTGTAAAGTG | |
| | ATGGGAGTGGAGGGCTGCACCAAGTGCATCAAGTACCTGCTCTTCGTCTTCAATTTCGTCTTC | |
| | TGGCTGGCTGGAGGCGTGATCCTGGGTGTGGCCCTGTGGCTCCGCCATGACCCGCAGACCACC | |
| | AACCTCCTGTATCTGGAGCTGGGAGACAAGCCCGCGCCCAACACCTTCTATGTAGGCATCTAC | |
| | ATCCTCATCGCTGTGGGCGCTGTCATGATGTTCGTTGGCTTCCTGGGCTGCTACGGGGCCATC | |
| | CAGGAATCCCAGTGCCTGCTGGGGACGTTCTTCACCTGCCTGGTCATCCTGTTTGCCTGTGAG | |
| | GTGGCCGCCGGCATCTGGGGCTTTGTCAACAAGGACCAGATCGCCAAGGATGTGAAGCAGTTC | |
| | TATGACCAGGCCCTACAGCAGGCCGTGGTGGATGATGACGCCAACAACGCCAAGGCTGTGGTG | |
| | AAGACCTTCCACGAGACGCTTGACTGCTGTGGCTCCAGCACACTGACTGCTTTGACCACCTCA | |
| | GTGCTCAAGAACAATTTGTGTCCCTCGGGCAGCAACATCATCAGCAACCTCTTCAAGGAGGAC | |
| | TGCCACCAGAAGATCGATGACCTCTTCTCCGGGAAGCTGTACCTCATCGGCATTGCTGCCATC | |
| | GTGGTCGCTGTGATCATGATCTTCGAGATGATCCTGAGCATGGTGCTGTGCTGTGGCATCCGG | |
| | AACAGCTCCGTGTACTGA | |

TABLE 13

| | | SEQ |
|---|---|---|
| | Sequence | ID NO: |
| Signal peptide | MARSVTLVFLVLVSLTGLYA | 1 |
| of β₂ | ATGGCTCGCTCGGTGACCCTGGTCTTTCTGGTGCTTGTCTCACTGACCGGCCTGTATGCT | 2 |
| microglobulin | | |
| OVA | SIINFEKL | 3 |
| peptide 1 | TCCATTATAAATTTTGAAAAGTTG | 4 |
| (for MHC class | | |
| I molecule) | | |
| Peptide linker 1 | GGGASGGGGSGGGGS | 5 |
| | GGCGGAGGTGCCTCTGGCGGTGGGGGCAGCGGTGGAGGGGGCAGT | 6 |
| β₂ | IQKTPQIQVYSRHPPENGKPNILNCYVTQFHPPHIEIQMLKNGKKIPKVEMSDMSFSKDWSFY | 7 |
| Microglobulin | ILAHTEFTPTETDTYACRVKHASMAEPKTVYWDRDM | |
| (from which | ATCCAGAAAACCCCTCAAATTCAAGTATACTCACGCCACCCACCGGAGAATGGGAAGCCGAAC | 8 |
| signal peptide | ATACTGAACTGCTACGTAACACAGTTCCACCCGCCTCACATTGAAATCCAAATGCTGAAGAAC | |
| is removed) | GGGAAAAAAATTCCTAAAGTAGAGATGTCAGATATGTCCTTCAGCAAGGACTGGTCTTTCTAT | |
| | ATCCTGGCTCACACTGAATTCACCCCCACTGAGACTGATACATACGCCTGCAGAGTTAAGCAT | |
| | GCCAGTATGGCCGAGCCCAAGACCGTCTACTGGGATCGAGACATG | |
| MHC class | GPHSLRYFVTAVSRPGLGEPRYMEVGYVDDTEFVRFDSDAENPRYEPRARWMEQEGPEYWERE | 9 |
| Iα chain | TQKAKGNEQSFRVDLRTLLGYYNQSKGGSHTIQVISGCEVGSDGRLLRGYQQYAYDGCDYIAL | |
| (from which | NEDLKTWTAADMAALITKHKWEQAGEAERLRAYLEGTCVEWLRRYLKNGNATLLRTDSPKAHV | |
| signal peptide | THHSRPEDKVTLRCWALGFYPADITLTWQLNGEELIQDMELVETRPAGDGTFQKWASVVVPLG | |
| is removed) | KEQYYTCHVYHQGLPEPLTLRWEPPPSTVSNMATVAVLVVLGAAIVTGAVVAFVMKMRRRNTG | |
| | GKGGDYALAPGSQTSDLSLPDCKVMVHDPHSLA | |
| | GGCCCACACTCGCTGAGGTATTTCGTCACCGCCGTGTCCCGGCCCGGCCTCGGGGAGCCCCGG | 10 |
| | TACATGGAAGTCGGCTACGTGGACGACACGGAGTTCGTGCGCTTCGACAGCGACGCGGAGAAT | |
| | CCGAGATATGAGCCGCGGGCGCGGTGGATGGAGCAGGAGGGGCCCGAGTATTGGGAGCGGGAG | |
| | ACACAGAAAGCCAAGGGCAATGAGCAGAGTTTCCGAGTGGACCTGAGGACCCTGCTCGGCTAC | |
| | TACAACCAGAGCAAGGGCGGCTCTCACACTATTCAGGTGATCTCTGGCTGTGAAGTGGGGTCC | |
| | GACGGGCGACTCCTCCGCGGGTACCAGCAGTACGCCTACGACGGCTGCGATTACATCGCCCTG | |
| | AACGAAGACCTGAAAACGTGGACGGCGGCGGACATGGCGGCGCTGATCACCAAACACAAGTGG | |
| | GAGCAGGCTGGTGAAGCAGAGAGACTCAGGGCCTACCTGGAGGGCACGTGCGTGGAGTGGCTC | |
| | CGCAGATACCTGAAGAACGGGAACGCGACGCTGCTGCGCACAGATTCCCCAAAGGCCCATGTG | |
| | ACCCATCACAGCAGACCTGAAGATAAAGTCACCCTGAGGTGCTGGGCCCTGGGCTTCTACCCT | |
| | GCTGACATCACCCTGACCTGGCAGTTGAATGGGGAGGAGCTGATCCAGGACATGGAGCTTGTG | |
| | GAGACCAGGCCTGCAGGGGATGGAACCTTCCAGAAGTGGGCATCTGTGGTGGTGCCTCTTGGG | |
| | AAGGAGCAGTATTACACATGCCATGTGTACCATCAGGGGCTGCCTGAGCCCCTCACCCTGAGA | |
| | TGGGAGCCTCCTCCATCCACTGTCTCCAACATGGCGACCGTTGCTGTTCTGGTTGTCCTTGGA | |
| | GCTGCAATAGTCACTGGAGCTGTGGTGGCTTTTGTGATGAAGATGAGAAGGAGAAACACAGGT | |
| | GGAAAAGGAGGGGACTATGCTCTGGCTCCAGGCTCCCAGACCTCTGATCTGTCTCTCCCAGAT | |
| | TGTAAAGTGATGGTTCATGACCCTCATTCTCTAGCG | |
| Peptide linker 2 | GGGGSGGGGSGGGGSGGGGS | 11 |
| | GGGGGGGGAGGCTCCGGTGGAGGGGGGTCTGGAGGGGGGGGGTCTGGTGGAGGCGGAAGT | 12 |
| Single chain | IQKTPQIQVYSRHPPENGKPNILNCYVTQFHPPHIEIQMLKNGKKIPKVEMSDMSFSKDWSFY | 65 |
| MHC class | ILAHTEFTPTETDTYACRVKHASMAEPKTVYWDRDMGGGGSGGGGSGGGGSGGGGSGPHSLRY | |
| I molecule | FVTAVSRPGLGEPRYMEVGYVDDTEFVRFDSDAENPRYEPRARWMEQEGPEYWERETQKAKGN | |
| (β₂ | EQSFRVDLRTLLGYYNQSKGGSHTIQVISGCEVGSDGRLLRGYQQYAYDGCDYIALNEDLKTW | |
| microglobulin | TAADMAALITKHKWEQAGEAERLRAYLEGTCVEWLRRYLKNGNATLLRTDSPKAHVTHHSRPE | |
| (from which | DKVTLRCWALGFYPADITLTWQLNGEELIQDMELVETRPAGDGTFQKWASVVVPLGKEQYYTC | |
| signal peptide | HVYHQGLPEPLTLRWEPPPSTVSNMATVAVLVVLGAAIVTGAVVAFVMKMRRRNTGGKGGDYA | |
| is removed) + | LAPGSQTSDLSLPDCKVMVHDPHSLA | |
| peptide linker | ATCCAGAAAACCCCTCAAATTCAAGTATACTCACGCCACCCACCGGAGAATGGGAAGCCGAAC | 66 |
| 2 + MHC class | ATACTGAACTGCTACGTAACACAGTTCCACCCGCCTCACATTGAAATCCAAATGCTGAAGAAC | |
| Iα chain | GGGAAAAAAATTCCTAAAGTAGAGATGTCAGATATGTCCTTCAGCAAGGACTGGTCTTTCTAT | |
| (from which | ATCCTGGCTCACACTGAATTCACCCCCACTGAGACTGATACATACGCCTGCAGAGTTAAGCAT | |
| signal peptide | GCCAGTATGGCCGAGCCCAAGACCGTCTACTGGGATCGAGACATGGGGGGGGGAGGCTCCGGT | |
| is removed)) | GGAGGGGGGTCTGGAGGGGGGGGGTCTGGTGGAGGCGGAAGTGGCCCACACTCGCTGAGGTAT | |
| | TTCGTCACCGCCGTGTCCCGGCCCGGCCTCGGGGAGCCCCGGTACATGGAAGTCGGCTACGTG | |
| | GACGACACGGAGTTCGTGCGCTTCGACAGCGACGCGGAGAATCCGAGATATGAGCCGCGGGCG | |
| | CGGTGGATGGAGCAGGAGGGGCCCGAGTATTGGGAGCGGGAGACACAGAAAGCCAAGGGCAAT | |
| | GAGCAGAGTTTCCGAGTGGACCTGAGGACCCTGCTCGGCTACTACAACCAGAGCAAGGGCGGC | |
| | TCTCACACTATTCAGGTGATCTCTGGCTGTGAAGTGGGGTCCGACGGGCGACTCCTCCGCGGG | |
| | TACCAGCAGTACGCCTACGACGGCTGCGATTACATCGCCCTGAACGAAGACCTGAAAACGTGG | |
| | ACGGCGGCGGACATGGCGGCGCTGATCACCAAACACAAGTGGGAGCAGGCTGGTGAAGCAGAG | |
| | AGACTCAGGGCCTACCTGGAGGGCACGTGCGTGGAGTGGCTCCGCAGATACCTGAAGAACGGG | |
| | AACGCGACGCTGCTGCGCACAGATTCCCCAAAGGCCCATGTGACCCATCACAGCAGACCTGAA | |
| | GATAAAGTCACCCTGAGGTGCTGGGCCCTGGGCTTCTACCCTGCTGACATCACCCTGACCTGG | |
| | CAGTTGAATGGGGAGGAGCTGATCCAGGACATGGAGCTTGTGGAGACCAGGCCTGCAGGGGAT | |
| | GGAACCTTCCAGAAGTGGGCATCTGTGGTGGTGCCTCTTGGGAAGGAGCAGTATTACACATGC | |
| | CATGTGTACCATCAGGGGCTGCCTGAGCCCCTCACCCTGAGATGGGAGCCTCCTCCATCCACT | |
| | GTCTCCAACATGGCGACCGTTGCTGTTCTGGTTGTCCTTGGAGCTGCAATAGTCACTGGAGCT | |
| | GTGGTGGCTTTTGTGATGAAGATGAGAAGGAGAAACACAGGTGGAAAAGGAGGGGACTATGCT | |
| | CTGGCTCCAGGCTCCCAGACCTCTGATCTGTCTCTCCCAGATTGTAAAGTGATGGTTCATGAC | |
| | CCTCATTCTCTAGCG | |
| sc-Trimer | MARSVTLVFLVLVSLTGLYASIINFEKLGGGASGGGGSGGGGSIQKTPQIQVYSRHPPENGKP | 13 |
| (OVA peptide 1 + | NILNCYVTQFHPPHIEIQMLKNGKKIPKVEMSDMSFSKDWSFYILAHTEFTPTETDTYACRVK | |
| peptide linker | HASMAEPKTVYWDRDMGGGGSGGGGSGGGGSGGGGSGPHSLRYFVTAVSRPGLGEPRYMEVGY | |
| 1 + single chain | VDDTEFVRFDSDAENPRYEPRARWMEQEGPEYWERETQKAKGNEQSFRVDLRTLLGYYNQSKG | |
| MHC class | GSHTIQVISGCEVGSDGRLLRGYQQYAYDGCDYIALNEDLKTWTAADMAALITKHKWEQAGEA | |
| I molecule) | ERLRAYLEGTCVEWLRRYLKNGNATLLRTDSPKAHVTHHSRPEDKVTLRCWALGFYPADITLT | |
| | WQLNGEELIQDMELVETRPAGDGTFQKWASVVVPLGKEQYYTCHVYHQGLPEPLTLRWEPPPS | |
| | TVSNMATVAVLVVLGAAIVTGAVVAFVMKMRRRNTGGKGGDYALAPGSQTSDLSLPDCKVMVH | |
| | DPHSLA | |
| | ATGGCTCGCTCGGTGACCCTGGTCTTTCTGGTGCTTGTCTCACTGACCGGCCTGTATGCTTCC | 14 |
| | ATTATAAATTTTGAAAAGTTGGGCGGAGGTGCCTCTGGCGGTGGGGGCAGCGGTGGAGGGGGC | |
| | AGTATCCAGAAAACCCCTCAAATTCAAGTATACTCACGCCACCCACCGGAGAATGGGAAGCCG | |
| | AACATACTGAACTGCTACGTAACACAGTTCCACCCGCCTCACATTGAAATCCAAATGCTGAAG | |
| | AACGGGAAAAAAATTCCTAAAGTAGAGATGTCAGATATGTCCTTCAGCAAGGACTGGTCTTTC | |
| | TATATCCTGGCTCACACTGAATTCACCCCCACTGAGACTGATACATACGCCTGCAGAGTTAAG | |
| | CATGCCAGTATGGCCGAGCCCAAGACCGTCTACTGGGATCGAGACATGGGGGGGGGAGGCTCC | |
| | GGTGGAGGGGGGTCTGGAGGGGGGGGGTCTGGTGGAGGCGGAAGTGGCCCACACTCGCTGAGG | |
| | TATTTCGTCACCGCCGTGTCCCGGCCCGGCCTCGGGGAGCCCCGGTACATGGAAGTCGGCTAC | |
| | GTGGACGACACGGAGTTCGTGCGCTTCGACAGCGACGCGGAGAATCCGAGATATGAGCCGCGG | |
| | GCGCGGTGGATGGAGCAGGAGGGGCCCGAGTATTGGGAGCGGGAGACACAGAAAGCCAAGGGC | |
| | AATGAGCAGAGTTTCCGAGTGGACCTGAGGACCCTGCTCGGCTACTACAACCAGAGCAAGGGC | |
| | GGCTCTCACACTATTCAGGTGATCTCTGGCTGTGAAGTGGGGTCCGACGGGCGACTCCTCCGC | |
| | GGGTACCAGCAGTACGCCTACGACGGCTGCGATTACATCGCCCTGAACGAAGACCTGAAAACG | |
| | TGGACGGCGGCGGACATGGCGGCGCTGATCACCAAACACAAGTGGGAGCAGGCTGGTGAAGCA | |
| | GAGAGACTCAGGGCCTACCTGGAGGGCACGTGCGTGGAGTGGCTCCGCAGATACCTGAAGAAC | |
| | GGGAACGCGACGCTGCTGCGCACAGATTCCCCAAAGGCCCATGTGACCCATCACAGCAGACCT | |
| | GAAGATAAAGTCACCCTGAGGTGCTGGGCCCTGGGCTTCTACCCTGCTGACATCACCCTGACC | |
| | TGGCAGTTGAATGGGGAGGAGCTGATCCAGGACATGGAGCTTGTGGAGACCAGGCCTGCAGGG | |
| | GATGGAACCTTCCAGAAGTGGGCATCTGTGGTGGTGCCTCTTGGGAAGGAGCAGTATTACACA | |
| | TGCCATGTGTACCATCAGGGGCTGCCTGAGCCCCTCACCCTGAGATGGGAGCCTCCTCCATCC | |
| | ACTGTCTCCAACATGGCGACCGTTGCTGTTCTGGTTGTCCTTGGAGCTGCAATAGTCACTGGA | |
| | GCTGTGGTGGCTTTTGTGATGAAGATGAGAAGGAGAAACACAGGTGGAAAAGGAGGGGACTAT | |
| | GCTCTGGCTCCAGGCTCCCAGACCTCTGATCTGTCTCTCCCAGATTGTAAAGTGATGGTTCAT | |
| | GACCCTCATTCTCTAGCG | |
| IL-2 | APTSSSTSSSTAEAQQQQQQQQQQQQHLEQLLMDLQELLSRMENYRNLKLPRMLTFKFYLPKQ | 25 |
| (from which | ATELKDLQCLEDELGPLRHVLDLTQSKSFQLEDAENFISNIRVTVVKLKGSDNTFECQFDDES | |
| signal peptide | ATVVDFLRRWIAFCQSIISTSPQ | |
| is removed) | GCACCCACTTCAAGCTCCACTTCAAGCTCTACAGCGGAAGCACAGCAGCAGCAGCAGCAGCAG | 26 |
| | CAGCAGCAGCAGCAGCACCTGGAGCAGCTGTTGATGGACCTACAGGAGCTCCTGAGCAGGATG | |
| | GAGAATTACAGGAACCTGAAACTCCCCAGGATGCTCACCTTCAAATTTTACTTGCCCAAGCAG | |
| | GCCACAGAATTGAAAGATCTTCAGTGCCTAGAAGATGAACTTGGACCTCTGCGGCATGTTCTG | |
| | GATTTGACTCAAAGCAAAAGCTTTCAATTGGAAGATGCTGAGAATTTCATCAGCAATATCAGA | |
| | GTAACTGTTGTAAAACTAAAGGGCTCTGACAACACATTTGAGTGCCAATTCGATGATGAGTCA | |
| | GCAACTGTGGTGGACTTTCTGAGGAGATGGATAGCCTTCTGTCAAAGCATCATCTCAACAAGC | |
| | CCTCAA | |
| CD81 | MGVEGCTKCIKYLLFVFNFVFWLAGGVILGVALWLRHDPQTTSLLYLELGNKPAPNTFYVGIY | 61 |
| (amino acids | ILIAVGAVMMFVGFLGCYGAIQESQCLLGTFFTCLVILFACEVAAGIWGFVNKDQIAKDVKQF | |
| 1 to 177) | YDQALQQAVMDDDANNAKAVVKTFHETLNCCGSNALTTLTTTILRNSLCPS | |
| | ATGGGGGTGGAGGGCTGCACCAAATGCATCAAATACCTGCTCTTCGTCTTCAATTTCGTCTTC | 62 |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCA | |
| CD81 | GGNILTPLLQQDCHQKIDELFSGKLYLIGIAAIVVAVIMIFEMILSMVLCCGIRNSSVY | 63 |
| (amino acids | GGCGGCAACATACTCACCCCCTTACTGCAGCAAGATTGTCATCAGAAAATCGATGAGCTCTTC | 64 |
| 178 to 236) | TCTGGGAAGCTGTACCTCATTGGAATTGCAGCCATTGTGGTAGCTGTCATTATGATCTTTGAG | |
| | ATGATTCTGAGCATGGTGCTGTGCTGTGGCATCCGGAACAGCTCCGTGTACTGA | |
| CD81-IL-2 | MGVEGCTKCIKYLLFVFNFVFWLAGGVILGVALWLRHDPQTTSLLYLELGNKPAPNTFYVGIY | 133 |
| | ILIAVGAVMMFVGFLGCYGAIQESQCLLGTFFTCLVILFACEVAAGIWGFVNKDQIAKDVKQF | |
| | YDQALQQAVMDDDANNAKAVVKTFHETLNCCGSNALTTLTTTILRNSLCPSGGGGSAPTSSST | |
| | SSSTAEAQQQQQQQQQQQQHLEQLLMDLQELLSRMENYRNLKLPRMLTFKFYLPKQATELKDL | |
| | QCLEDELGPLRHVLDLTQSKSFQLEDAENFISNIRVTVVKLKGSDNTFECQFDDESATVVDFL | |
| | RRWIAFCQSIISTSPQGGGGSGGNILTPLLQQDCHQKIDELFSGKLYLIGIAAIVVAVIMIFE | |
| | MILSMVLCCGIRNSSVY | |
| | ATGGGGGTGGAGGGCTGCACCAAATGCATCAAATACCTGCTCTTCGTCTTCAATTTCGTCTTC | 134 |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCAGGAGGAGGAGGAAGCGCACCCACTTCAAGCTCCACT | |
| | TCAAGCTCTACAGCGGAAGCACAGCAGCAGCAGCAGCAGCAGCAGCAGCAGCAGCAGCACCTG | |
| | GAGCAGCTGTTGATGGACCTACAGGAGCTCCTGAGCAGGATGGAGAATTACAGGAACCTGAAA | |
| | CTCCCCAGGATGCTCACCTTCAAATTTTACTTGCCCAAGCAGGCCACAGAATTGAAAGATCTT | |
| | CAGTGCCTAGAAGATGAACTTGGACCTCTGCGGCATGTTCTGGATTTGACTCAAAGCAAAAGC | |
| | TTTCAATTGGAAGATGCTGAGAATTTCATCAGCAATATCAGAGTAACTGTTGTAAAACTAAAG | |
| | GGCTCTGACAACACATTTGAGTGCCAATTCGATGATGAGTCAGCAACTGTGGTGGACTTTCTG | |
| | AGGAGATGGATAGCCTTCTGTCAAAGCATCATCTCAACAAGCCCTCAAGGAGGAGGAGGAAGC | |
| | GGCGGCAACATACTCACCCCCTTACTGCAGCAAGATTGTCATCAGAAAATCGATGAGCTCTTC | |
| | TCTGGGAAGCTGTACCTCATTGGAATTGCAGCCATTGTGGTAGCTGTCATTATGATCTTTGAG | |
| | ATGATTCTGAGCATGGTGCTGTGCTGTGGCATCCGGAACAGCTCCGTGTACTGA | |
| sc-Trimer- | MARSVTLVFLVLVSLTGLYASIINFEKLGGGASGGGGSGGGGSIQKTPQIQVYSRHPPENGKP | 135 |
| CD81-IL-2 | NILNCYVTQFHPPHIEIQMLKNGKKIPKVEMSDMSFSKDWSFYILAHTEFTPTETDTYACRVK | |
| (sc-Trimer + | HASMAEPKTVYWDRDMGGGGSGGGGSGGGGSGGGGSGPHSLRYFVTAVSRPGLGEPRYMEVGY | |
| CD81) | VDDTEFVRFDSDAENPRYEPRARWMEQEGPEYWERETQKAKGNEQSFRVDLRTLLGYYNQSKG | |
| | GSHTIQVISGCEVGSDGRLLRGYQQYAYDGCDYIALNEDLKTWTAADMAALITKHKWEQAGEA | |
| | ERLRAYLEGTCVEWLRRYLKNGNATLLRTDSPKAHVTHHSRPEDKVTLRCWALGFYPADITLT | |
| | WQLNGEELIQDMELVETRPAGDGTFQKWASVVVPLGKEQYYTCHVYHQGLPEPLTLRWEPPPS | |
| | TVSNMATVAVLVVLGAAIVTGAVVAFVMKMRRRNTGGKGGDYALAPGSQTSDLSLPDCKVMVH | |
| | DPHSLAMGVEGCTKCIKYLLFVFNFVFWLAGGVILGVALWLRHDPQTTSLLYLELGNKPAPNT | |
| | FYVGIYILIAVGAVMMFVGFLGCYGAIQESQCLLGTFFTCLVILFACEVAAGIWGFVNKDQIA | |
| | KDVKQFYDQALQQAVMDDDANNAKAVVKTFHETLNCCGSNALTTLTTTILRNSLCPSGGGGSA | |
| | PTSSSTSSSTAEAQQQQQQQQQQQQHLEQLLMDLQELLSRMENYRNLKLPRMLTFKFYLPKQA | |
| | TELKDLQCLEDELGPLRHVLDLTQSKSFQLEDAENFISNIRVTVVKLKGSDNTFECQFDDESA | |
| | TVVDFLRRWIAFCQSIISTSPQGGGGSGGNILTPLLQQDCHQKIDELFSGKLYLIGIAAIVVA | |
| | VIMIFEMILSMVLCCGIRNSSVY | |
| | ATGGCTCGCTCGGTGACCCTGGTCTTTCTGGTGCTTGTCTCACTGACCGGCCTGTATGCTTCC | 136 |
| | ATTATAAATTTTGAAAAGTTGGGCGGAGGTGCCTCTGGCGGTGGGGGCAGCGGTGGAGGGGGC | |
| | AGTATCCAGAAAACCCCTCAAATTCAAGTATACTCACGCCACCCACCGGAGAATGGGAAGCCG | |
| | AACATACTGAACTGCTACGTAACACAGTTCCACCCGCCTCACATTGAAATCCAAATGCTGAAG | |
| | AACGGGAAAAAAATTCCTAAAGTAGAGATGTCAGATATGTCCTTCAGCAAGGACTGGTCTTTC | |
| | TATATCCTGGCTCACACTGAATTCACCCCCACTGAGACTGATACATACGCCTGCAGAGTTAAG | |
| | CATGCCAGTATGGCCGAGCCCAAGACCGTCTACTGGGATCGAGACATGGGGGGGGGAGGCTCC | |
| | GGTGGAGGGGGGTCTGGAGGGGGGGGGTCTGGTGGAGGCGGAAGTGGCCCACACTCGCTGAGG | |
| | TATTTCGTCACCGCCGTGTCCCGGCCCGGCCTCGGGGAGCCCCGGTACATGGAAGTCGGCTAC | |
| | GTGGACGACACGGAGTTCGTGCGCTTCGACAGCGACGCGGAGAATCCGAGATATGAGCCGCGG | |
| | GCGCGGTGGATGGAGCAGGAGGGGCCCGAGTATTGGGAGCGGGAGACACAGAAAGCCAAGGGC | |
| | AATGAGCAGAGTTTCCGAGTGGACCTGAGGACCCTGCTCGGCTACTACAACCAGAGCAAGGGC | |
| | GGCTCTCACACTATTCAGGTGATCTCTGGCTGTGAAGTGGGGTCCGACGGGCGACTCCTCCGC | |
| | GGGTACCAGCAGTACGCCTACGACGGCTGCGATTACATCGCCCTGAACGAAGACCTGAAAACG | |
| | TGGACGGCGGCGGACATGGCGGCGCTGATCACCAAACACAAGTGGGAGCAGGCTGGTGAAGCA | |
| | GAGAGACTCAGGGCCTACCTGGAGGGCACGTGCGTGGAGTGGCTCCGCAGATACCTGAAGAAC | |
| | GGGAACGCGACGCTGCTGCGCACAGATTCCCCAAAGGCCCATGTGACCCATCACAGCAGACCT | |
| | GAAGATAAAGTCACCCTGAGGTGCTGGGCCCTGGGCTTCTACCCTGCTGACATCACCCTGACC | |
| | TGGCAGTTGAATGGGGAGGAGCTGATCCAGGACATGGAGCTTGTGGAGACCAGGCCTGCAGGG | |
| | GATGGAACCTTCCAGAAGTGGGCATCTGTGGTGGTGCCTCTTGGGAAGGAGCAGTATTACACA | |
| | TGCCATGTGTACCATCAGGGGCTGCCTGAGCCCCTCACCCTGAGATGGGAGCCTCCTCCATCC | |
| | ACTGTCTCCAACATGGCGACCGTTGCTGTTCTGGTTGTCCTTGGAGCTGCAATAGTCACTGGA | |
| | GCTGTGGTGGCTTTTGTGATGAAGATGAGAAGGAGAAACACAGGTGGAAAAGGAGGGGACTAT | |
| | GCTCTGGCTCCAGGCTCCCAGACCTCTGATCTGTCTCTCCCAGATTGTAAAGTGATGGTTCAT | |
| | GACCCTCATTCTCTAGCGATGGGGGTGGAGGGCTGCACCAAATGCATCAAATACCTGCTCTTC | |
| | GTCTTCAATTTCGTCTTCTGGCTGGCTGGAGGCGTGATCCTAGGTGTAGCTCTGTGGTTGCGT | |
| | CATGATCCACAGACCACCAGCCTGCTGTACCTGGAACTGGGAAACAAACCGGCACCCAACACC | |
| | TTCTACGTGGGCATCTACATTCTCATTGCTGTGGGAGCTGTGATGATGTTTGTAGGCTTCCTG | |
| | GGGTGCTATGGGGCCATCCAGGAGTCCCAGTGTCTGCTGGGGACGTTCTTCACCTGCCTTGTG | |
| | ATCCTGTTTGCCTGTGAGGTGGCTGCAGGCATCTGGGGCTTCGTAAACAAAGACCAGATCGCC | |
| | AAGGATGTGAAGCAGTTCTATGACCAGGCCCTTCAGCAAGCTGTGATGGATGATGATGCCAAC | |
| | AATGCCAAGGCTGTGGTGAAGACTTTCCATGAGACGCTCAACTGTTGTGGCTCCAACGCACTG | |
| | ACCACACTGACTACCACCATACTGAGGAACAGCCTGTGTCCCTCAGGAGGAGGAGGAAGCGCA | |
| | CCCACTTCAAGCTCCACTTCAAGCTCTACAGCGGAAGCACAGCAGCAGCAGCAGCAGCAGCAG | |
| | CAGCAGCAGCAGCACCTGGAGCAGCTGTTGATGGACCTACAGGAGCTCCTGAGCAGGATGGAG | |
| | AATTACAGGAACCTGAAACTCCCCAGGATGCTCACCTTCAAATTTTACTTGCCCAAGCAGGCC | |
| | ACAGAATTGAAAGATCTTCAGTGCCTAGAAGATGAACTTGGACCTCTGCGGCATGTTCTGGAT | |
| | TTGACTCAAAGCAAAAGCTTTCAATTGGAAGATGCTGAGAATTTCATCAGCAATATCAGAGTA | |
| | ACTGTTGTAAAACTAAAGGGCTCTGACAACACATTTGAGTGCCAATTCGATGATGAGTCAGCA | |
| | ACTGTGGTGGACTTTCTGAGGAGATGGATAGCCTTCTGTCAAAGCATCATCTCAACAAGCCCT | |
| | CAAGGAGGAGGAGGAAGCGGCGGCAACATACTCACCCCCTTACTGCAGCAAGATTGTCATCAG | |
| | AAAATCGATGAGCTCTTCTCTGGGAAGCTGTACCTCATTGGAATTGCAGCCATTGTGGTAGCT | |
| | GTCATTATGATCTTTGAGATGATTCTGAGCATGGTGCTGTGCTGTGGCATCCGGAACAGCTCC | |
| | GTGTACTGA | |
| Aka-Luc | MEDAKNIKKGPAPFYPLEDGTAGEQLHKAMKRYALVPGAIAFTDAHIQVDVTYAEYFEMSVRL | 137 |
| | AEAMRRYGLNTNHRIVVCSENSSQFFMPVLGALFIGVAVAPANDIYNERELLNSMGISQPTVV | |
| | FVSKKGLRKVLNVQKKLPIIRKIIIMDSKTDYQGFQSMYTFVTSHLPPSFNEYDFVPESFDRD | |
| | KTIALIMNSSGSTGLPKGVALPHRTACVRFSHARDPIFGYQNIPDTAILSVVPFHHGFGMFTT | |
| | LGYLICGFRVVLMYRFEEELFLRSLQDYKIQSALLVPTLFSCLAKSTLIDKYDLSSLREIASG | |
| | GAPLSKEVGEAVAKRFRLPGIRQGYGLTETTNAVMITPEGDRKPGSVGKVVPFFEAKVVDLVT | |
| | GKTLGVNQRGELCVRGPMIMSGYVNNPEATNALIDKDGWLHSGDIAYWDEDEHFFIVDRLKSL | |
| | IKYKGYQVAPAELEGILLQHPYIFDAGVAGLPDDDAGELPAAVVVLEHGKTMTEKEIVDYVAS | |
| | QVTTAKKLRGGVVFVDEVPRGSTGKLDARKIREILTKAKKDGKIAV | |
| | ATGGAAGATGCCAAAAACATTAAGAAGGGCCCAGCGCCGTTCTACCCACTCGAAGACGGGACC | 138 |
| | GCCGGCGAGCAGCTGCACAAAGCCATGAAGCGCTACGCCCTGGTGCCCGGCGCCATCGCCTTT | |
| | ACCGACGCACATATTCAGGTGGACGTTACCTACGCCGAGTACTTCGAGATGAGCGTTCGGCTG | |
| | GCAGAAGCTATGAGGCGCTATGGGCTGAATACAAACCATCGGATCGTGGTGTGCAGCGAGAAT | |
| | AGCTCGCAGTTCTTCATGCCCGTGTTGGGTGCCCTGTTCATCGGTGTGGCTGTGGCCCCAGCT | |
| | AACGACATCTACAACGAGCGCGAGCTGCTGAACAGCATGGGCATCAGCCAGCCCACCGTCGTA | |
| | TTCGTGAGCAAGAAAGGGCTGCGAAAGGTCCTCAACGTGCAAAAGAAGCTACCGATCATACGA | |
| | AAGATCATCATCATGGATAGCAAGACCGACTACCAGGGCTTCCAAAGCATGTACACCTTCGTG | |
| | ACTTCCCATTTGCCACCCAGCTTCAACGAGTACGACTTCGTGCCCGAGAGCTTCGACCGGGAC | |
| | AAAACCATCGCCCTGATCATGAACAGTAGTGGTAGTACAGGATTACCCAAGGGCGTAGCCCTA | |
| | CCGCACCGCACCGCTTGTGTCCGATTCAGTCATGCCCGCGACCCCATCTTCGGCTACCAGAAC | |
| | ATCCCCGACACCGCTATCCTCAGCGTGGTGCCATTTCACCACGGCTTCGGCATGTTCACCACG | |
| | CTGGGCTACTTGATCTGCGGCTTTCGGGTCGTGCTCATGTACCGCTTCGAGGAGGAGCTATTC | |
| | TTGCGCAGCTTGCAAGACTATAAGATTCAATCTGCCCTGCTGGTGCCCACACTATTTAGCTGC | |
| | CTCGCTAAGAGCACTCTCATCGACAAGTACGACCTAAGCAGCTTGCGCGAGATCGCCAGCGGC | |
| | GGGGCGCCGCTCAGCAAGGAGGTAGGTGAGGCCGTGGCCAAACGCTTCCGCCTACCAGGCATC | |
| | CGCCAGGGCTATGGCCTGACAGAAACAACCAACGCCGTCATGATCACCCCCGAGGGGGACCGT | |
| | AAGCCTGGCTCAGTAGGCAAGGTGGTGCCCTTCTTCGAGGCTAAGGTGGTAGACTTGGTCACC | |
| | GGTAAGACACTGGGTGTGAACCAGCGCGGTGAGCTGTGCGTCCGTGGCCCCATGATCATGAGC | |
| | GGCTACGTTAACAACCCCGAGGCTACGAACGCTCTCATCGACAAGGACGGCTGGCTGCACAGC | |
| | GGCGACATCGCCTACTGGGACGAGGACGAGCACTTCTTCATCGTGGACCGGCTGAAGAGCCTG | |
| | ATCAAATACAAGGGCTACCAGGTAGCCCCAGCCGAACTGGAGGGCATCCTGCTGCAACACCCC | |
| | TACATCTTCGACGCCGGAGTCGCCGGCCTGCCCGACGACGATGCCGGCGAGCTGCCCGCCGCA | |
| | GTCGTCGTGTTGGAACACGGTAAAACCATGACCGAGAAAGAGATCGTGGACTATGTGGCCAGC | |
| | CAGGTTACAACCGCCAAGAAGCTGCGCGGTGGTGTTGTGTTTGTGGATGAAGTCCCTAGAGGA | |
| | TCGACCGGCAAGTTAGACGCCCGCAAGATCCGCGAGATTCTCACTAAGGCCAAGAAGGACGGC | |
| | AAGATCGCCGTG | |
| CD63 | MAVEGGMKCVKFLLYVLLLAFCACAVGLIAIGVAVQVVLKQAITHETTAGSLLPVVIIAVGAF | 27 |
| | LFLVAFVGCCGACKENYCLMITFAIFLSLIMLVEVAVAIAGYVFRDQVKSEFNKSFQQQMQNY | |
| | LKDNKTATILDKLQKENNCCGASNYTDWENIPGMAKDRVPDSCCINITVGCGNDFKESTIHTQ | |
| | GCVETIAIWLRKNILLVAAAALGIAFVEVLGIIFSCCLVKSIRSGYEVM | |
| | ATGGCGGTGGAAGGAGGAATGAAGTGTGTCAAGTTTTTGCTCTACGTTCTCCTGCTGGCCTTC | 28 |
| | TGCGCCTGTGCAGTGGGATTGATCGCCATTGGTGTAGCGGTTCAGGTTGTCTTGAAGCAGGCC | |
| | ATTACCCATGAGACTACTGCTGGCTCGCTGTTGCCTGTGGTCATCATTGCAGTGGGTGCCTTC | |
| | CTCTTCCTGGTGGCCTTTGTGGGCTGCTGTGGGGCCTGCAAGGAGAACTACTGTCTCATGATT | |
| | ACATTTGCCATCTTCCTGTCTCTTATCATGCTTGTGGAGGTGGCTGTGGCCATTGCTGGCTAT | |
| | GTGTTTAGAGACCAGGTGAAGTCAGAGTTTAATAAAAGCTTCCAGCAGCAGATGCAGAATTAC | |
| | CTTAAAGACAACAAAACAGCCACTATTTTGGACAAATTGCAGAAAGAAAATAACTGCTGTGGA | |
| | GCTTCTAACTACACAGACTGGGAAAACATCCCCGGCATGGCCAAGGACAGAGTCCCCGATTCT | |
| | TGCTGCATCAACATAACTGTGGGCTGTGGGAATGATTTCAAGGAATCCACTATCCATACCCAG | |
| | GGCTGCGTGGAGACTATAGCAATATGGCTAAGGAAGAACATACTGCTGGTGGCTGCAGCGGCC | |
| | CTGGGCATTGCTTTTGTGGAGGTCTTGGGAATTATCTTCTCCTGCTGTCTGGTGAAGAGTATT | |
| | CGAAGTGGCTATGAAGTAATGTAG | |
| CD63 | MAVEGGMKCVKFLLYVLLLAFCACAVGLIAIGVAVQVVLKQAITHETTAGSLLPVVIIAVGAF | 57 |
| (amino acids | LFLVAFVGCCGACKENYCLMITFAIFLSLIMLVEVAVAIAGYVFRDQVKSEFNKSFQQQMQNY | |
| 1 to 170) | LKDNKTATILDKLQKENNCCGASNYTDWENIPGMAKDRVPDSCC | |
| | ATGGCGGTGGAAGGAGGAATGAAGTGTGTCAAGTTTTTGCTCTACGTTCTCCTGCTGGCCTTC | 58 |
| | TGCGCCTGTGCAGTGGGATTGATCGCCATTGGTGTAGCGGTTCAGGTTGTCTTGAAGCAGGCC | |
| | ATTACCCATGAGACTACTGCTGGCTCGCTGTTGCCTGTGGTCATCATTGCAGTGGGTGCCTTC | |
| | CTCTTCCTGGTGGCCTTTGTGGGCTGCTGTGGGGCCTGCAAGGAGAACTACTGTCTCATGATT | |
| | ACATTTGCCATCTTCCTGTCTCTTATCATGCTTGTGGAGGTGGCTGTGGCCATTGCTGGCTAT | |
| | GTGTTTAGAGACCAGGTGAAGTCAGAGTTTAATAAAAGCTTCCAGCAGCAGATGCAGAATTAC | |
| | CTTAAAGACAACAAAACAGCCACTATTTTGGACAAATTGCAGAAAGAAAATAACTGCTGTGGA | |
| | GCTTCTAACTACACAGACTGGGAAAACATCCCCGGCATGGCCAAGGACAGAGTCCCCGATTCT | |
| | TGCTGC | |
| CD63 | INITVGCGNDFKESTIHTQGCVETIAIWLRKNILLVAAAALGIAFVEVLGIIFSCCLVKSIRS | 59 |
| (amino acids | GYEVM | |
| 171 to 238) | ATCAACATAACTGTGGGCTGTGGGAATGATTTCAAGGAATCCACTATCCATACCCAGGGCTGC | 60 |
| | GTGGAGACTATAGCAATATGGCTAAGGAAGAACATACTGCTGGTGGCTGCAGCGGCCCTGGGC | |
| | ATTGCTTTTGTGGAGGTCTTGGGAATTATCTTCTCCTGCTGTCTGGTGAAGAGTATTCGAAGT | |
| | GGCTATGAAGTAATGTAG | |
| Peptide linker 3 | GGGGS | 29 |
| | GGAGGAGGAGGAAGC | 30 |
| CD63-Aka- | MAVEGGMKCVKFLLYVLLLAFCACAVGLIAIGVAVQVVLKQAITHETTAGSLLPVVIIAVGAF | 139 |
| Luc | LFLVAFVGCCGACKENYCLMITFAIFLSLIMLVEVAVAIAGYVFRDQVKSEFNKSFQQQMQNY | 140 |
| | LKDNKTATILDKLQKENNCCGASNYTDWENIPGMAKDRVPDSCCGGGGSMEDAKNIKKGPAPF | |
| | YPLEDGTAGEQLHKAMKRYALVPGAIAFTDAHIQVDVTYAEYFEMSVRLAEAMRRYGLNTNHR | |
| | IVVCSENSSQFFMPVLGALFIGVAVAPANDIYNERELLNSMGISQPTVVFVSKKGLRKVLNVQ | |
| | KKLPIIRKIIIMDSKTDYQGFQSMYTFVTSHLPPSFNEYDFVPESFDRDKTIALIMNSSGSTG | |
| | LPKGVALPHRTACVRFSHARDPIFGYQNIPDTAILSVVPFHHGFGMFTTLGYLICGFRVVLMY | |
| | RFEEELFLRSLQDYKIQSALLVPTLFSCLAKSTLIDKYDLSSLREIASGGAPLSKEVGEAVAK | |
| | RFRLPGIRQGYGLTETTNAVMITPEGDRKPGSVGKVVPFFEAKVVDLVTGKTLGVNQRGELCV | |
| | RGPMIMSGYVNNPEATNALIDKDGWLHSGDIAYWDEDEHFFIVDRLKSLIKYKGYQVAPAELE | |
| | GILLQHPYIFDAGVAGLPDDDAGELPAAVVVLEHGKTMTEKEIVDYVASQVTTAKKLRGGVVF | |
| | VDEVPRGSTGKLDARKIREILTKAKKDGKIAVGGGGSINITVGCGNDFKESTIHTQGCVETIA | |
| | IWLRKNILLVAAAALGIAFVEVLGIIFSCCLVKSIRSGYEVM | |
| | ATGGCGGTGGAAGGAGGAATGAAGTGTGTCAAGTTTTTGCTCTACGTTCTCCTGCTGGCCTTC | |
| | TGCGCCTGTGCAGTGGGATTGATCGCCATTGGTGTAGCGGTTCAGGTTGTCTTGAAGCAGGCC | |
| | ATTACCCATGAGACTACTGCTGGCTCGCTGTTGCCTGTGGTCATCATTGCAGTGGGTGCCTTC | |
| | CTCTTCCTGGTGGCCTTTGTGGGCTGCTGTGGGGCCTGCAAGGAGAACTACTGTCTCATGATT | |
| | ACATTTGCCATCTTCCTGTCTCTTATCATGCTTGTGGAGGTGGCTGTGGCCATTGCTGGCTAT | |
| | GTGTTTAGAGACCAGGTGAAGTCAGAGTTTAATAAAAGCTTCCAGCAGCAGATGCAGAATTAC | |
| | CTTAAAGACAACAAAACAGCCACTATTTTGGACAAATTGCAGAAAGAAAATAACTGCTGTGGA | |
| | GCTTCTAACTACACAGACTGGGAAAACATCCCCGGCATGGCCAAGGACAGAGTCCCCGATTCT | |
| | TGCTGCGGTGGTGGTGGTTCTATGGAAGATGCCAAAAACATTAAGAAGGGCCCAGCGCCGTTC | |
| | TACCCACTCGAAGACGGGACCGCCGGCGAGCAGCTGCACAAAGCCATGAAGCGCTACGCCCTG | |
| | GTGCCCGGCGCCATCGCCTTTACCGACGCACATATTCAGGTGGACGTTACCTACGCCGAGTAC | |
| | TTCGAGATGAGCGTTCGGCTGGCAGAAGCTATGAGGCGCTATGGGCTGAATACAAACCATCGG | |
| | ATCGTGGTGTGCAGCGAGAATAGCTCGCAGTTCTTCATGCCCGTGTTGGGTGCCCTGTTCATC | |
| | GGTGTGGCTGTGGCCCCAGCTAACGACATCTACAACGAGCGCGAGCTGCTGAACAGCATGGGC | |
| | ATCAGCCAGCCCACCGTCGTATTCGTGAGCAAGAAAGGGCTGCGAAAGGTCCTCAACGTGCAA | |
| | AAGAAGCTACCGATCATACGAAAGATCATCATCATGGATAGCAAGACCGACTACCAGGGCTTC | |
| | CAAAGCATGTACACCTTCGTGACTTCCCATTTGCCACCCAGCTTCAACGAGTACGACTTCGTG | |
| | CCCGAGAGCTTCGACCGGGACAAAACCATCGCCCTGATCATGAACAGTAGTGGTAGTACAGGA | |
| | TTACCCAAGGGCGTAGCCCTACCGCACCGCACCGCTTGTGTCCGATTCAGTCATGCCCGCGAC | |
| | CCCATCTTCGGCTACCAGAACATCCCCGACACCGCTATCCTCAGCGTGGTGCCATTTCACCAC | |
| | GGCTTCGGCATGTTCACCACGCTGGGCTACTTGATCTGCGGCTTTCGGGTCGTGCTCATGTAC | |
| | CGCTTCGAGGAGGAGCTATTCTTGCGCAGCTTGCAAGACTATAAGATTCAATCTGCCCTGCTG | |
| | GTGCCCACACTATTTAGCTGCCTCGCTAAGAGCACTCTCATCGACAAGTACGACCTAAGCAGC | |
| | TTGCGCGAGATCGCCAGCGGCGGGGCGCCGCTCAGCAAGGAGGTAGGTGAGGCCGTGGCCAAA | |
| | CGCTTCCGCCTACCAGGCATCCGCCAGGGCTATGGCCTGACAGAAACAACCAACGCCGTCATG | |
| | ATCACCCCCGAGGGGGACCGTAAGCCTGGCTCAGTAGGCAAGGTGGTGCCCTTCTTCGAGGCT | |
| | AAGGTGGTAGACTTGGTCACCGGTAAGACACTGGGTGTGAACCAGCGCGGTGAGCTGTGCGTC | |
| | CGTGGCCCCATGATCATGAGCGGCTACGTTAACAACCCCGAGGCTACGAACGCTCTCATCGAC | |
| | AAGGACGGCTGGCTGCACAGCGGCGACATCGCCTACTGGGACGAGGACGAGCACTTCTTCATC | |
| | GTGGACCGGCTGAAGAGCCTGATCAAATACAAGGGCTACCAGGTAGCCCCAGCCGAACTGGAG | |
| | GGCATCCTGCTGCAACACCCCTACATCTTCGACGCCGGAGTCGCCGGCCTGCCCGACGACGAT | |
| | GCCGGCGAGCTGCCCGCCGCAGTCGTCGTGTTGGAACACGGTAAAACCATGACCGAGAAAGAG | |
| | ATCGTGGACTATGTGGCCAGCCAGGTTACAACCGCCAAGAAGCTGCGCGGTGGTGTTGTGTTT | |
| | GTGGATGAAGTCCCTAGAGGATCGACCGGCAAGTTAGACGCCCGCAAGATCCGCGAGATTCTC | |
| | ACTAAGGCCAAGAAGGACGGCAAGATCGCCGTGGGTGGTGGTGGTTCTATCAACATAACTGTG | |
| | GGCTGTGGGAATGATTTCAAGGAATCCACTATCCATACCCAGGGCTGCGTGGAGACTATAGCA | |
| | ATATGGCTAAGGAAGAACATACTGCTGGTGGCTGCAGCGGCCCTGGGCATTGCTTTTGTGGAG | |
| | GTCTTGGGAATTATCTTCTCCTGCTGTCTGGTGAAGAGTATTCGAAGTGGCTATGAAGTAATG | |
| | TAG | |

TABLE 14

| | | SEQ |
|---|---|---|
| | Sequence | ID NO: |
| Signal peptide | MSRSVALAVLALLSLSGLEA | 121 |
| of hß₂ | ATGTCTCGCTCCGTGGCCTTAGCTGTGCTCGCGCTACTCTCTCTTTCTGGCCTGGAGGCT | 122 |
| microglobulin | | |
| SARS-CoV2 | KLWAQCVQL | 141 |
| peptide 1 | AAACTGTGGGCCCAGTGTGTGCAGCTG | 142 |
| (for MHC class | | |
| I molecule) | | |
| Peptide linker 1 | GGGASGGGGSGGGGS | 5 |
| | GGCGGAGGTGCCTCTGGCGGTGGGGGCAGCGGTGGAGGGGGCAGT | 6 |
| hß₂ | IQRTPKIQVYSRHPAENGKSNFLNCYVSGFHPSDIEVDLLKNGERIEKVEHSDLSFSKDWSFY | 121 |
| Microglobulin | LLYYTEFTPTEKDEYACRVNHVTLSQPKIVKWDRDM | |
| (from which | ATCCAGCGTACTCCAAAGATTCAGGTTTACTCACGTCATCCAGCAGAGAATGGAAAGTCAAAT | 122 |
| signal peptide | TTCCTGAATTGCTATGTGTCTGGGTTTCATCCATCCGACATTGAAGTTGACTTACTGAAGAAT | |
| is removed) | GGAGAGAGAATTGAAAAAGTGGAGCATTCAGACTTGTCTTTCAGCAAGGACTGGTCTTTCTAT | |
| | CTCTTGTACTACACTGAATTCACCCCCACTGAAAAAGATGAGTATGCCTGCCGTGTGAACCAT | |
| | GTGACTTTGTCACAGCCCAAGATAGTTAAGTGGGATCGAGACATG | |
| hMHC class | GSHSMRYFFTSVSRPGRGEPRFIAVGYVDDTQFVRFDSDAASQRMEPRAPWIEQEGPEYWDGE | 143 |
| I (HLA-A0201) | TRKVKAHSQTHRVDLGTLRGYYNQSEAGSHTVQRMYGCDVGSDWRFLRGYHQYAYDGKDYIAL | |
| α chain | KEDLRSWTAADMAAQTTKHKWEAAHVAEQLRAYLEGTCVEWLRRYLENGKETLQRTDAPKTHM | |
| (from which | THHAVSDHEATLRCWALSFYPAEITLTWQRDGEDQTQDTELVETRPAGDGTFQKWAAVVVPSG | |
| signal peptide | QEQRYTCHVQHEGLPKPLTLRWEPSSQPTIPIVGIIAGLVLFGAVITGAVVAAVMWRRKSSDR | |
| is removed) | KGGSYSQAASSDSAQGSDVSLTACKV | |
| | GGCTCTCACTCCATGAGGTATTTCTTCACATCCGTGTCCCGGCCCGGCCGCGGGGAGCCCCGC | 144 |
| | TTCATCGCAGTGGGCTACGTGGACGACACGCAGTTCGTGCGGTTCGACAGCGACGCCGCGAGC | |
| | CAGAGGATGGAGCCGCGGGCGCCGTGGATAGAGCAGGAGGGTCCGGAGTATTGGGACGGGGAG | |
| | ACACGGAAAGTGAAGGCCCACTCACAGACTCACCGAGTGGACCTGGGGACCCTGCGCGGCTAC | |
| | TACAACCAGAGCGAGGCCGGTTCTCACACCGTCCAGAGGATGTATGGCTGCGACGTGGGGTCG | |
| | GACTGGCGCTTCCTCCGCGGGTACCACCAGTACGCCTACGACGGCAAGGATTACATCGCCCTG | |
| | AAAGAGGACCTGCGCTCTTGGACCGCGGCGGACATGGCAGCTCAGACCACCAAGCACAAGTGG | |
| | GAGGCGGCCCATGTGGCGGAGCAGTTGAGAGCCTACCTGGAGGGCACGTGCGTGGAGTGGCTC | |
| | CGCAGATACCTGGAGAACGGGAAGGAGACGCTGCAGCGCACGGACGCCCCCAAAACGCATATG | |
| | ACTCACCACGCTGTCTCTGACCATGAAGCCACCCTGAGGTGCTGGGCCCTGAGCTTCTACCCT | |
| | GCGGAGATCACACTGACCTGGCAGCGGGATGGGGAGGACCAGACCCAGGACACGGAGCTCGTG | |
| | GAGACCAGGCCTGCAGGGGATGGAACCTTCCAGAAGTGGGCGGCTGTGGTGGTGCCTTCTGGA | |
| | CAGGAGCAGAGATACACCTGCCATGTGCAGCATGAGGGTTTGCCCAAGCCCCTCACCCTGAGA | |
| | TGGGAGCCGTCTTCCCAGCCCACCATCCCCATCGTGGGCATCATTGCTGGCCTGGTTCTCTTT | |
| | GGAGCTGTGATCACTGGAGCTGTGGTCGCTGCTGTGATGTGGAGGAGGAAGAGCTCAGATAGA | |
| | AAAGGAGGGAGCTACTCTCAGGCTGCAAGCAGTGACAGTGCCCAGGGCTCTGATGTGTCTCTC | |
| | ACAGCTTGTAAAGTG | |
| Peptide linker 2 | GGGGSGGGGSGGGGSGGGGS | 11 |
| | GGGGGGGGAGGCTCCGGTGGAGGGGGGTCTGGAGGGGGGGGGTCTGGTGGAGGCGGAAGT | 12 |
| h single chain | IQRTPKIQVYSRHPAENGKSNFLNCYVSGFHPSDIEVDLLKNGERIEKVEHSDLSFSKDWSFY | 145 |
| MHC class | LLYYTEFTPTEKDEYACRVNHVTLSQPKIVKWDRDMGGGGSGGGGSGGGGSGGGGSGSHSMRY | |
| I molecule | FFTSVSRPGRGEPRFIAVGYVDDTQFVRFDSDAASQRMEPRAPWIEQEGPEYWDGETRKVKAH | |
| (ß₂ | SQTHRVDLGTLRGYYNQSEAGSHTVQRMYGCDVGSDWRFLRGYHQYAYDGK | |
| microglobulin | DYIALKEDLRSWTAADMAAQTTKHKWEAAHVAEQLRAYLEGTCVEWLRRYLENGKETLQRTDA | |
| (from which | PKTHMTHHAVSDHEATLRCWALSFYPAEITLTWQRDGEDQTQDTELVETRPAGDGTFQKWAAV | |
| signal peptide | VVPSGQEQRYTCHVQHEGLPKPLTLRWEPSSQPTIPIVGIIAGLVLFGAVITGAVVAAVMWRR | |
| is removed) + | KSSDRKGGSYSQAASSDSAQGSDVSLTACKV | |
| peptide linker | ATCCAGCGTACTCCAAAGATTCAGGTTTACTCACGTCATCCAGCAGAGAATGGAAAGTCAAAT | 146 |
| 2 + MHC class I | TTCCTGAATTGCTATGTGTCTGGGTTTCATCCATCCGACATTGAAGTTGACTTACTGAAGAAT | |
| (HLA-A0201) | GGAGAGAGAATTGAAAAAGTGGAGCATTCAGACTTGTCTTTCAGCAAGGACTGGTCTTTCTAT | |
| α chain | CTCTTGTACTACACTGAATTCACCCCCACTGAAAAAGATGAGTATGCCTGCCGTGTGAACCAT | |
| (from which | GTGACTTTGTCACAGCCCAAGATAGTTAAGTGGGATCGAGACATGGGAGGCGGCGGGTCTGGC | |
| signal peptide | GGCGGCGGCAGTGGCGGTGGCGGTAGCGGCGGAGGTGGATCTGGCTCTCACTCCATGAGGTAT | |
| is removed)) | TTCTTCACATCCGTGTCCCGGCCCGGCCGCGGGGAGCCCCGCTTCATCGCAGTGGGCTACGTG | |
| | GACGACACGCAGTTCGTGCGGTTCGACAGCGACGCCGCGAGCCAGAGGATGGAGCCGCGGGCG | |
| | CCGTGGATAGAGCAGGAGGGTCCGGAGTATTGGGACGGGGAGACACGGAAAGTGAAGGCCCAC | |
| | TCACAGACTCACCGAGTGGACCTGGGGACCCTGCGCGGCTACTACAACCAGAGCGAGGCCGGT | |
| | TCTCACACCGTCCAGAGGATGTATGGCTGCGACGTGGGGTCGGACTGGCGCTTCCTCCGCGGG | |
| | TACCACCAGTACGCCTACGACGGCAAGGATTACATCGCCCTGAAAGAGGACCTGCGCTCTTGG | |
| | ACCGCGGCGGACATGGCAGCTCAGACCACCAAGCACAAGTGGGAGGCGGCCCATGTGGCGGAG | |
| | CAGTTGAGAGCCTACCTGGAGGGCACGTGCGTGGAGTGGCTCCGCAGATACCTGGAGAACGGG | |
| | AAGGAGACGCTGCAGCGCACGGACGCCCCCAAAACGCATATGACTCACCACGCTGTCTCTGAC | |
| | CATGAAGCCACCCTGAGGTGCTGGGCCCTGAGCTTCTACCCTGCGGAGATCACACTGACCTGG | |
| | CAGCGGGATGGGGAGGACCAGACCCAGGACACGGAGCTCGTGGAGACCAGGCCTGCAGGGGAT | |
| | GGAACCTTCCAGAAGTGGGCGGCTGTGGTGGTGCCTTCTGGACAGGAGCAGAGATACACCTGC | |
| | CATGTGCAGCATGAGGGTTTGCCCAAGCCCCTCACCCTGAGATGGGAGCCGTCTTCCCAGCCC | |
| | ACCATCCCCATCGTGGGCATCATTGCTGGCCTGGTTCTCTTTGGAGCTGTGATCACTGGAGCT | |
| | GTGGTCGCTGCTGTGATGTGGAGGAGGAAGAGCTCAGATAGAAAAGGAGGGAGCTACTCTCAG | |
| | GCTGCAAGCAGTGACAGTGCCCAGGGCTCTGATGTGTCTCTCACAGCTTGTAAAGTG | |
| hsc-Trimer | MSRSVALAVLALLSLSGLEAKLWAQCVQLGGGASGGGGSGGGGSIQRTPKIQVYSRHPAENGK | 147 |
| (SARS-CoV2 | SNFLNCYVSGFHPSDIEVDLLKNGERIEKVEHSDLSFSKDWSFYLLYYTEFTPTEKDEYACRV | |
| peptide 1 + | NHVTLSQPKIVKWDRDMGGGGSGGGGSGGGGSGGGGSGSHSMRYFFTSVSRPGRGEPRFIAVG | |
| peptide linker | YVDDTQFVRFDSDAASQRMEPRAPWIEQEGPEYWDGETRKVKAHSQTHRVDLGTLRGYYNQSE | |
| 1 + single chain | AGSHTVQRMYGCDVGSDWRFLRGYHQYAYDGKDYIALKEDLRSWTAADMAAQTTKHKWEAAHV | |
| MHC class I | AEQLRAYLEGTCVEWLRRYLENGKETLQRTDAPKTHMTHHAVSDHEATLRCWALSFYPAEITL | |
| (HLA-A0201) | TWQRDGEDQTQDTELVETRPAGDGTFQKWAAVVVPSGQEQRYTCHVQHEGLPKPLTLRWEPSS | |
| molecule) | QPTIPIVGIIAGLVLFGAVITGAVVAAVMWRRKSSDRKGGSYSQAASSDSAQGSDVSLTACKV | |
| | ATGTCTCGCTCCGTGGCCTTAGCTGTGCTCGCGCTACTCTCTCTTTCTGGCCTGGAGGCTAAA | 148 |
| | CTGTGGGCCCAGTGTGTGCAGCTGGGCGGAGGGGCATCAGGCGGCGGTGGGTCAGGTGGAGGT | |
| | GGGAGTATCCAGCGTACTCCAAAGATTCAGGTTTACTCACGTCATCCAGCAGAGAATGGAAAG | |
| | TCAAATTTCCTGAATTGCTATGTGTCTGGGTTTCATCCATCCGACATTGAAGTTGACTTACTG | |
| | AAGAATGGAGAGAGAATTGAAAAAGTGGAGCATTCAGACTTGTCTTTCAGCAAGGACTGGTCT | |
| | TTCTATCTCTTGTACTACACTGAATTCACCCCCACTGAAAAAGATGAGTATGCCTGCCGTGTG | |
| | AACCATGTGACTTTGTCACAGCCCAAGATAGTTAAGTGGGATCGAGACATGGGAGGCGGCGGG | |
| | TCTGGCGGCGGCGGCAGTGGCGGTGGCGGTAGCGGCGGAGGTGGATCTGGCTCTCACTCCATG | |
| | AGGTATTTCTTCACATCCGTGTCCCGGCCCGGCCGCGGGGAGCCCCGCTTCATCGCAGTGGGC | |
| | TACGTGGACGACACGCAGTTCGTGCGGTTCGACAGCGACGCCGCGAGCCAGAGGATGGAGCCG | |
| | CGGGCGCCGTGGATAGAGCAGGAGGGTCCGGAGTATTGGGACGGGGAGACACGGAAAGTGAAG | |
| | GCCCACTCACAGACTCACCGAGTGGACCTGGGGACCCTGCGCGGCTACTACAACCAGAGCGAG | |
| | GCCGGTTCTCACACCGTCCAGAGGATGTATGGCTGCGACGTGGGGTCGGACTGGCGCTTCCTC | |
| | CGCGGGTACCACCAGTACGCCTACGACGGCAAGGATTACATCGCCCTGAAAGAGGACCTGCGC | |
| | TCTTGGACCGCGGCGGACATGGCAGCTCAGACCACCAAGCACAAGTGGGAGGCGGCCCATGTG | |
| | GCGGAGCAGTTGAGAGCCTACCTGGAGGGCACGTGCGTGGAGTGGCTCCGCAGATACCTGGAG | |
| | AACGGGAAGGAGACGCTGCAGCGCACGGACGCCCCCAAAACGCATATGACTCACCACGCTGTC | |
| | TCTGACCATGAAGCCACCCTGAGGTGCTGGGCCCTGAGCTTCTACCCTGCGGAGATCACACTG | |
| | ACCTGGCAGCGGGATGGGGAGGACCAGACCCAGGACACGGAGCTCGTGGAGACCAGGCCTGCA | |
| | GGGGATGGAACCTTCCAGAAGTGGGCGGCTGTGGTGGTGCCTTCTGGACAGGAGCAGAGATAC | |
| | ACCTGCCATGTGCAGCATGAGGGTTTGCCCAAGCCCCTCACCCTGAGATGGGAGCCGTCTTCC | |
| | CAGCCCACCATCCCCATCGTGGGCATCATTGCTGGCCTGGTTCTCTTTGGAGCTGTGATCACT | |
| | GGAGCTGTGGTCGCTGCTGTGATGTGGAGGAGGAAGAGCTCAGATAGAAAAGGAGGGAGCTAC | |
| | TCTCAGGCTGCAAGCAGTGACAGTGCCCAGGGCTCTGATGTGTCTCTCACAGCTTGTAAAGTG | |
| hCD81 | MGVEGCTKCIKYLLFVFNFVFWLAGGVILGVALWLRHDPQTTNLLYLELGDKPAPNTFYVGIY | 129 |
| | ILIAVGAVMMFVGFLGCYGAIQESQCLLGTFFTCLVILFACEVAAGIWGFVNKDQIAKDVKQF | |
| | YDQALQQAVVDDDANNAKAVVKTFHETLDCCGSSTLTALTTSVLKNNLCPSGSNIISNLFKED | |
| | CHQKIDDLFSGKLYLIGIAAIVVAVIMIFEMILSMVLCCGIRNSSVY | |
| | ATGGGAGTGGAGGGCTGCACCAAGTGCATCAAGTACCTGCTCTTCGTCTTCAATTTCGTCTTC | 130 |
| | TGGCTGGCTGGAGGCGTGATCCTGGGTGTGGCCCTGTGGCTCCGCCATGACCCGCAGACCACC | |
| | AACCTCCTGTATCTGGAGCTGGGAGACAAGCCCGCGCCCAACACCTTCTATGTAGGCATCTAC | |
| | ATCCTCATCGCTGTGGGCGCTGTCATGATGTTCGTTGGCTTCCTGGGCTGCTACGGGGCCATC | |
| | CAGGAATCCCAGTGCCTGCTGGGGACGTTCTTCACCTGCCTGGTCATCCTGTTTGCCTGTGAG | |
| | GTGGCCGCCGGCATCTGGGGCTTTGTCAACAAGGACCAGATCGCCAAGGATGTGAAGCAGTTC | |
| | TATGACCAGGCCCTACAGCAGGCCGTGGTGGATGATGACGCCAACAACGCCAAGGCTGTGGTG | |
| | AAGACCTTCCACGAGACGCTTGACTGCTGTGGCTCCAGCACACTGACTGCTTTGACCACCTCA | |
| | GTGCTCAAGAACAATTTGTGTCCCTCGGGCAGCAACATCATCAGCAACCTCTTCAAGGAGGAC | |
| | TGCCACCAGAAGATCGATGACCTCTTCTCCGGGAAGCTGTACCTCATCGGCATTGCTGCCATC | |
| | GTGGTCGCTGTGATCATGATCTTCGAGATGATCCTGAGCATGGTGCTGTGCTGTGGCATCCGG | |
| | AACAGCTCCGTGTACTGA | |
| hsc-Trimer- | MSRSVALAVLALLSLSGLEAKLWAQCVQLGGGASGGGGSGGGGSIQRTPKIQVYSRHPAENGK | 149 |
| CD81 | SNFLNCYVSGFHPSDIEVDLLKNGERIEKVEHSDLSFSKDWSFYLLYYTEFTPTEKDEYACRV | |
| (SARS-CoV2 | NHVTLSQPKIVKWDRDMGGGGSGGGGSGGGGSGGGGSGSHSMRYFFTSVSRPGRGEPRFIAVG | |
| sc-Trimer + | YVDDTQFVRFDSDAASQRMEPRAPWIEQEGPEYWDGETRKVKAHSQTHRVDLGTLRGYYNQSE | |
| CD81) | AGSHTVQRMYGCDVGSDWRFLRGYHQYAYDGKDYIALKEDLRSWTAADMAAQTTKHKWEAAHV | |
| | AEQLRAYLEGTCVEWLRRYLENGKETLQRTDAPKTHMTHHAVSDHEATLRCWALSFYPAEITL | |
| | TWQRDGEDQTQDTELVETRPAGDGTFQKWAAVVVPSGQEQRYTCHVQHEGLPKPLTLRWEPSS | |
| | QPTIPIVGIIAGLVLFGAVITGAVVAAVMWRRKSSDRKGGSYSQAASSDSAQGSDVSLTACKV | |
| | MGVEGCTKCIKYLLFVFNFVFWLAGGVILGVALWLRHDPQTTNLLYLELGDKPAPNTFYVGIY | |
| | ILIAVGAVMMFVGFLGCYGAIQESQCLLGTFFTCLVILFACEVAAGIWGFVNKDQIAKDVKQF | |
| | YDQALQQAVVDDDANNAKAVVKTFHETLDCCGSSTLTALTTSVLKNNLCPSGSNIISNLFKED | |
| | CHQKIDDLFSGKLYLIGIAAIVVAVIMIFEMILSMVLCCGIRNSSVY | |
| | ATGTCTCGCTCCGTGGCCTTAGCTGTGCTCGCGCTACTCTCTCTTTCTGGCCTGGAGGCTAAA | 150 |
| | CTGTGGGCCCAGTGTGTGCAGCTGGGCGGAGGGGCATCAGGCGGCGGTGGGTCAGGTGGAGGT | |
| | GGGAGTATCCAGCGTACTCCAAAGATTCAGGTTTACTCACGTCATCCAGCAGAGAATGGAAAG | |
| | TCAAATTTCCTGAATTGCTATGTGTCTGGGTTTCATCCATCCGACATTGAAGTTGACTTACTG | |
| | AAGAATGGAGAGAGAATTGAAAAAGTGGAGCATTCAGACTTGTCTTTCAGCAAGGACTGGTCT | |
| | TTCTATCTCTTGTACTACACTGAATTCACCCCCACTGAAAAAGATGAGTATGCCTGCCGTGTG | |
| | AACCATGTGACTTTGTCACAGCCCAAGATAGTTAAGTGGGATCGAGACATGGGAGGCGGCGGG | |
| | TCTGGCGGCGGCGGCAGTGGCGGTGGCGGTAGCGGCGGAGGTGGATCTGGCTCTCACTCCATG | |
| | AGGTATTTCTTCACATCCGTGTCCCGGCCCGGCCGCGGGGAGCCCCGCTTCATCGCAGTGGGC | |
| | TACGTGGACGACACGCAGTTCGTGCGGTTCGACAGCGACGCCGCGAGCCAGAGGATGGAGCCG | |
| | CGGGCGCCGTGGATAGAGCAGGAGGGTCCGGAGTATTGGGACGGGGAGACACGGAAAGTGAAG | |
| | GCCCACTCACAGACTCACCGAGTGGACCTGGGGACCCTGCGCGGCTACTACAACCAGAGCGAG | |
| | GCCGGTTCTCACACCGTCCAGAGGATGTATGGCTGCGACGTGGGGTCGGACTGGCGCTTCCTC | |
| | CGCGGGTACCACCAGTACGCCTACGACGGCAAGGATTACATCGCCCTGAAAGAGGACCTGCGC | |
| | TCTTGGACCGCGGCGGACATGGCAGCTCAGACCACCAAGCACAAGTGGGAGGCGGCCCATGTG | |
| | GCGGAGCAGTTGAGAGCCTACCTGGAGGGCACGTGCGTGGAGTGGCTCCGCAGATACCTGGAG | |
| | AACGGGAAGGAGACGCTGCAGCGCACGGACGCCCCCAAAACGCATATGACTCACCACGCTGTC | |
| | TCTGACCATGAAGCCACCCTGAGGTGCTGGGCCCTGAGCTTCTACCCTGCGGAGATCACACTG | |
| | ACCTGGCAGCGGGATGGGGAGGACCAGACCCAGGACACGGAGCTCGTGGAGACCAGGCCTGCA | |
| | GGGGATGGAACCTTCCAGAAGTGGGCGGCTGTGGTGGTGCCTTCTGGACAGGAGCAGAGATAC | |
| | ACCTGCCATGTGCAGCATGAGGGTTTGCCCAAGCCCCTCACCCTGAGATGGGAGCCGTCTTCC | |
| | CAGCCCACCATCCCCATCGTGGGCATCATTGCTGGCCTGGTTCTCTTTGGAGCTGTGATCACT | |
| | GGAGCTGTGGTCGCTGCTGTGATGTGGAGGAGGAAGAGCTCAGATAGAAAAGGAGGGAGCTAC | |
| | TCTCAGGCTGCAAGCAGTGACAGTGCCCAGGGCTCTGATGTGTCTCTCACAGCTTGTAAAGTG | |
| | ATGGGAGTGGAGGGCTGCACCAAGTGCATCAAGTACCTGCTCTTCGTCTTCAATTTCGTCTTC | |
| | TGGCTGGCTGGAGGCGTGATCCTGGGTGTGGCCCTGTGGCTCCGCCATGACCCGCAGACCACC | |
| | AACCTCCTGTATCTGGAGCTGGGAGACAAGCCCGCGCCCAACACCTTCTATGTAGGCATCTAC | |
| | ATCCTCATCGCTGTGGGCGCTGTCATGATGTTCGTTGGCTTCCTGGGCTGCTACGGGGCCATC | |
| | CAGGAATCCCAGTGCCTGCTGGGGACGTTCTTCACCTGCCTGGTCATCCTGTTTGCCTGTGAG | |
| | GTGGCCGCCGGCATCTGGGGCTTTGTCAACAAGGACCAGATCGCCAAGGATGTGAAGCAGTTC | |
| | TATGACCAGGCCCTACAGCAGGCCGTGGTGGATGATGACGCCAACAACGCCAAGGCTGTGGTG | |
| | AAGACCTTCCACGAGACGCTTGACTGCTGTGGCTCCAGCACACTGACTGCTTTGACCACCTCA | |
| | GTGCTCAAGAACAATTTGTGTCCCTCGGGCAGCAACATCATCAGCAACCTCTTCAAGGAGGAC | |
| | TGCCACCAGAAGATCGATGACCTCTTCTCCGGGAAGCTGTACCTCATCGGCATTGCTGCCATC | |
| | GTGGTCGCTGTGATCATGATCTTCGAGATGATCCTGAGCATGGTGCTGTGCTGTGGCATCCGG | |
| | AACAGCTCCGTGTACTGA | |

TABLE 15-1

| | | SEQ |
|---|---|---|
| | Sequence | ID NO: |
| HLA DR1 | MVCLKLPGGSCMTALTVTLMVLSSPLALS | 151 |
| ß chain signal | ATGGTGTGCCTTAAACTCCCTGGCGGAAGCTGCATGACCGCCTTGACTGTGACCCTGATGGTT | 152 |
| sequence | CTGAGCTCTCCATTGGCCCTGAGT | |
| TPI peptide | GELIGILNAAKVPAD | 153 |
| | GGAGAGCTCATTGGCATCCTGAACGCTGCCAAGGTGCCCGCAGAC | 154 |
| Linker | GGGGSGGGGSG | 155 |
| | GGTGGCGGAGGGAGCGGAGGCGGTGGGTCCGGC | 156 |
| HLA DR1 | GDTRPRFLWQLKFECHFFNGTERVRLLERCIYNQEESVRFDSDVGEYRAVTELGRPDAEYWNS | 157 |
| ß chain | QKDLLEQRRAAVDTYCRHNYGVGESFTVQRRVEPKVTVYPSKTQPLQHHNLLVCSVSGFYPGS | |
| | IEVRWFRNGQEEKAGVVSTGLIQNGDWTFQTLVMLETVPRSGEVYTCQVEHPSVTSPLTVEWR | |
| | ARSESAQSKMLSGVGGFVLGLLFLGAGLFIYFRNQKGHSGLQPTGFLS | |
| | GGGGACACTAGACCACGATTTCTTTGGCAGTTGAAGTTCGAGTGCCACTTCTTCAATGGGACA | 158 |
| | GAGAGGGTCAGGCTTCTGGAACGCTGCATCTACAACCAAGAGGAGAGTGTGCGGTTTGACTCT | |
| | GATGTGGGCGAATATCGGGCAGTCACGGAACTTGGGCGTCCTGATGCCGAGTACTGGAATTCC | |
| | CAGAAGGATCTGCTGGAACAACGACGGGCTGCTGTTGACACGTACTGTCGCCACAACTATGGC | |
| | GTCGGTGAGAGCTTTACCGTGCAAAGGAGAGTAGAGCCCAAGGTGACCGTTTATCCCTCCAAG | |
| | ACCCAACCCTTGCAGCATCACAATCTGCTGGTATGTAGCGTGTCTGGCTTCTATCCTGGCAGC | |
| | ATAGAAGTCAGGTGGTTTCGGAACGGTCAGGAAGAGAAAGCTGGGGTGGTTAGCACAGGACTC | |
| | ATTCAGAATGGGGACTGGACATTCCAGACCCTGGTGATGCTGGAGACAGTACCGAGATCAGGA | |
| | GAGGTGTACACATGTCAGGTGGAACATCCCTCAGTGACTAGTCCACTCACTGTCGAATGGCGT | |
| | GCGAGATCCGAGTCAGCACAGAGCAAAATGCTCTCCGGAGTTGGCGGATTCGTCCTGGGACTG | |
| | CTGTTTCTCGGTGCCGGTCTGTTCATCTACTTCCGCAACCAGAAGGGCCATTCCGGCCTGCAG | |
| | CCTACTGGGTTTCTGTCT | |
| hCD81 | MGVEGCTKCIKYLLFVFNFVFWLAGGVILGVALWLRHDPQTTNLLYLELGDKPAPNTFYVGIY | 159 |
| | ILIAVGAVMMFVGFLGCYGAIQESQCLLGTFFTCLVILFACEVAAGIWGFVNKDQIAKDVKQF | |
| | YDQALQQAVVDDDANNAKAVVKTFHETLDCCGSSTLTALTTSVLKNNLCPSGSNIISNLFKED | |
| | CHQKIDDLFSGKLYLIGIAAIVVAVIMIFEMILSMVLCCGIRNSSVY | |
| | ATGGGAGTGGAGGGCTGCACCAAGTGCATCAAGTACCTGCTCTTCGTCTTCAATTTCGTCTTC | 160 |
| | TGGCTGGCTGGAGGCGTGATCCTGGGTGTGGCCCTGTGGCTCCGCCATGACCCGCAGACCACC | |
| | AACCTCCTGTATCTGGAGCTGGGAGACAAGCCCGCGCCCAACACCTTCTATGTAGGCATCTAC | |
| | ATCCTCATCGCTGTGGGCGCTGTCATGATGTTCGTTGGCTTCCTGGGCTGCTACGGGGCCATC | |
| | CAGGAATCCCAGTGCCTGCTGGGGACGTTCTTCACCTGCCTGGTCATCCTGTTTGCCTGTGAG | |
| | GTGGCCGCCGGCATCTGGGGCTTTGTCAACAAGGACCAGATCGCCAAGGATGTGAAGCAGTTC | |
| | TATGACCAGGCCCTACAGCAGGCCGTGGTGGATGATGACGCCAACAACGCCAAGGCTGTGGTG | |
| | AAGACCTTCCACGAGACGCTTGACTGCTGTGGCTCCAGCACACTGACTGCTTTGACCACCTCA | |
| | GTGCTCAAGAACAATTTGTGTCCCTCGGGCAGCAACATCATCAGCAACCTCTTCAAGGAGGAC | |
| | TGCCACCAGAAGATCGATGACCTCTTCTCCGGGAAGCTGTACCTCATCGGCATTGCTGCCATC | |
| | GTGGTCGCTGTGATCATGATCTTCGAGATGATCCTGAGCATGGTGCTGTGCTGTGGCATCCGG | |
| | AACAGCTCCGTGTAC | |
| P2A | GSGATNFSLLKQAGDVEENPGP | 161 |
| | GGATCTGGAGCCACGAACTTCTCTCTGTTAAAGCAAGCAGGAGACGTGGAAGAAAACCCCGGT | 162 |
| | CCT | |
| HLA DR1 | MAISGVPVLGFFIIAVLMSAQESWAIKEEHVIIQAEFYLNPDQSGEFMFDFDGDEIFHVDMAK | 163 |
| α chain | KETVWRLEEFGRFASFEAQGALANIAVDKANLEIMTKRSNYTPITNVPPEVTVLTNSPVELRE | |
| | PNVLICFIDKFTPPVVNVTWLRNGKPVTTGVSETVFLPREDHLFRKFHYLPFLPSTEDVYDCR | |
| | VEHWGLDEPLLKHWEFDAPSPLPETTENVVCALGLTVGLVGIIIGTIFIIKGVRKSNAAERRG | |
| | PL | |
| | ATGGCCATTAGCGGGGTTCCAGTGCTGGGCTTCTTCATCATAGCGGTACTGATGTCTGCCCAG | 164 |
| | GAATCCTGGGCGATCAAAGAAGAACACGTCATCATACAGGCAGAGTTCTACCTGAATCCCGAT | |
| | CAGTCCGGAGAGTTTATGTTCGACTTTGATGGGGACGAGATTTTCCATGTGGATATGGCTAAG | |
| | AAGGAAACCGTCTGGAGACTGGAAGAGTTTGGCCGTTTTGCCAGTTTTGAGGCCCAAGGAGCA | |
| | CTGGCCAACATAGCAGTCGACAAAGCCAACCTGGAGATTATGACGAAACGCAGCAACTATACG | |
| | CCCATTACCAATGTACCACCTGAAGTGACAGTGCTGACTAACTCACCCGTGGAATTGCGAGAA | |
| | CCCAATGTGCTGATCTGCTTCATCGACAAGTTCACTCCACCGGTTGTTAACGTCACTTGGCTT | |
| | CGGAATGGCAAGCCTGTCACTACCGGTGTCTCTGAGACAGTGTTTCTGCCGAGGGAGGATCAT | |
| | CTCTTTCGCAAGTTCCACTATCTTCCCTTTCTCCCTAGTACCGAAGATGTATACGATTGCAGA | |
| | GTGGAGCATTGGGGATTGGACGAGCCACTGCTGAAGCACTGGGAGTTCGACGCACCTTCACCC | |
| | CTTCCAGAGACAACCGAAAATGTGGTGTGTGCTCTCGGACTCACAGTTGGCTTGGTGGGCATT | |
| | ATCATTGGGACCATTTTCATCATCAAAGGGGTGAGGAAAAGCAACGCTGCTGAGCGGAGAGGT | |
| | CCTCTGTGA | |
| TPI peptide- | MVCLKLPGGSCMTALTVTLMVLSSPLALSGELIGILNAAKVPADGGGGSGGGGSGGDTRPRFL | 165 |
| HLA DR1 | WQLKFECHFFNGTERVRLLERCIYNQEESVRFDSDVGEYRAVTELGRPDAEYWNSQKDLLEQR | |
| ß chain-h | RAAVDTYCRHNYGVGESFTVQRRVEPKVTVYPSKTQPLQHHNLLVCSVSGFYPGSIEVRWFRN | |
| CD81-HLA | GQEEKAGVVSTGLIQNGDWTFQTLVMLETVPRSGEVYTCQVEHPSVTSPLTVEWRARSESAQS | |
| DR1α chain | KMLSGVGGFVLGLLFLGAGLFIYFRNQKGHSGLQPTGFLSMGVEGCTKCIKYLLFVFNFVFWL | |
| | AGGVILGVALWLRHDPQTTNLLYLELGDKPAPNTFYVGIYILIAVGAVMMFVGFLGCYGAIQE | |
| | SQCLLGTFFTCLVILFACEVAAGIWGFVNKDQIAKDVKQFYDQALQQAVVDDDANNAKAVVKT | |
| | FHETLDCCGSSTLTALTTSVLKNNLCPSGSNIISNLFKEDCHQKIDDLFSGKLYLIGIAAIVV | |
| | AVIMIFEMILSMVLCCGIRNSSVYGSGATNFSLLKQAGDVEENPGPMAISGVPVLGFFIIAVL | |
| | MSAQESWAIKEEHVIIQAEFYLNPDQSGEFMFDFDGDEIFHVDMAKKETVWRLEEFGRFASFE | |
| | AQGALANIAVDKANLEIMTKRSNYTPITNVPPEVTVLTNSPVELREPNVLICFIDKFTPPVVN | |
| | VTWLRNGKPVTTGVSETVFLPREDHLFRKFHYLPFLPSTEDVYDCRVEHWGLDEPLLKHWEFD | |
| | APSPLPETTENVVCALGLTVGLVGIIIGTIFIIKGVRKSNAAERRGPL | |
| | ATGGTGTGCCTTAAACTCCCTGGCGGAAGCTGCATGACCGCCTTGACTGTGACCCTGATGGTT | 166 |
| | CTGAGCTCTCCATTGGCCCTGAGTGGAGAGCTCATTGGCATCCTGAACGCTGCCAAGGTGCCC | |
| | GCAGACGGTGGCGGAGGGAGCGGAGGCGGTGGGTCCGGCGGGGACACTAGACCACGATTTCTT | |
| | TGGCAGTTGAAGTTCGAGTGCCACTTCTTCAATGGGACAGAGAGGGTCAGGCTTCTGGAACGC | |
| | ACGGAACTTGGGCGTCCTGATGCCGAGTACTGGAATTCCCAGAAGGATCTGCTGGAACAACGA | |
| | CGGGCTGCTGTTGACACGTACTGTCGCCACAACTATGGCGTCGGTGAGAGCTTTACCGTGCAA | |
| | AGGAGAGTAGAGCCCAAGGTGACCGTTTATCCCTCCAAGACCCAACCCTTGCAGCATCACAAT | |
| | CTGCTGGTATGTAGCGTGTCTGGCTTCTATCCTGGCAGCATAGAAGTCAGGTGGTTTCGGAAC | |
| | GGTCAGGAAGAGAAAGCTGGGGTGGTTAGCACAGGACTCATTCAGAATGGGGACTGGACATTC | |
| | CAGACCCTGGTGATGCTGGAGACAGTACCGAGATCAGGAGAGGTGTACACATGTCAGGTGGAA | |
| | CATCCCTCAGTGACTAGTCCACTCACTGTCGAATGGCGTGCGAGATCCGAGTCAGCACAGAGC | |
| | AAAATGCTCTCCGGAGTTGGCGGATTCGTCCTGGGACTGCTGTTTCTCGGTGCCGGTCTGTTC | |
| | ATCTACTTCCGCAACCAGAAGGGCCATTCCGGCCTGCAGCCTACTGGGTTTCTGTCTATGGGA | |
| | GTGGAGGGCTGCACCAAGTGCATCAAGTACCTGCTCTTCGTCTTCAATTTCGTCTTCTGGCTG | |
| | GCTGGAGGCGTGATCCTGGGTGTGGCCCTGTGGCTCCGCCATGACCCGCAGACCACCAACCTC | |
| | CTGTATCTGGAGCTGGGAGACAAGCCCGCGCCCAACACCTTCTATGTAGGCATCTACATCCTC | |
| | ATCGCTGTGGGCGCTGTCATGATGTTCGTTGGCTTCCTGGGCTGCTACGGGGCCATCCAGGAA | |
| | TCCCAGTGCCTGCTGGGGACGTTCTTCACCTGCCTGGTCATCCTGTTTGCCTGTGAGGTGGCC | |
| | GCCGGCATCTGGGGCTTTGTCAACAAGGACCAGATCGCCAAGGATGTGAAGCAGTTCTATGAC | |
| | CAGGCCCTACAGCAGGCCGTGGTGGATGATGACGCCAACAACGCCAAGGCTGTGGTGAAGACC | |
| | TTCCACGAGACGCTTGACTGCTGTGGCTCCAGCACACTGACTGCTTTGACCACCTCAGTGCTC | |
| | AAGAACAATTTGTGTCCCTCGGGCAGCAACATCATCAGCAACCTCTTCAAGGAGGACTGCCAC | |
| | CAGAAGATCGATGACCTCTTCTCCGGGAAGCTGTACCTCATCGGCATTGCTGCCATCGTGGTC | |
| | GCTGTGATCATGATCTTCGAGATGATCCTGAGCATGGTGCTGTGCTGTGGCATCCGGAACAGC | |
| | TCCGTGTACGGATCTGGAGCCACGAACTTCTCTCTGTTAAAGCAAGCAGGAGACGTGGAAGAA | |
| | AACCCCGGTCCTATGGCCATTAGCGGGGTTCCAGTGCTGGGCTTCTTCATCATAGCGGTACTG | |
| | ATGTCTGCCCAGGAATCCTGGGCGATCAAAGAAGAACACGTCATCATACAGGCAGAGTTCTAC | |
| | CTGAATCCCGATCAGTCCGGAGAGTTTATGTTCGACTTTGATGGGGACGAGATTTTCCATGTG | |
| | GATATGGCTAAGAAGGAAACCGTCTGGAGACTGGAAGAGTTTGGCCGTTTTGCCAGTTTTGAG | |
| | GCCCAAGGAGCACTGGCCAACATAGCAGTCGACAAAGCCAACCTGGAGATTATGACGAAACGC | |
| | AGCAACTATACGCCCATTACCAATGTACCACCTGAAGTGACAGTGCTGACTAACTCACCCGTG | |
| | GAATTGCGAGAACCCAATGTGCTGATCTGCTTCATCGACAAGTTCACTCCACCGGTTGTTAAC | |
| | GTCACTTGGCTTCGGAATGGCAAGCCTGTCACTACCGGTGTCTCTGAGACAGTGTTTCTGCCG | |
| | AGGGAGGATCATCTCTTTCGCAAGTTCCACTATCTTCCCTTTCTCCCTAGTACCGAAGATGTA | |
| | TACGATTGCAGAGTGGAGCATTGGGGATTGGACGAGCCACTGCTGAAGCACTGGGAGTTCGAC | |
| | GCACCTTCACCCCTTCCAGAGACAACCGAAAATGTGGTGTGTGCTCTCGGACTCACAGTTGGC | |
| | TTGGTGGGCATTATCATTGGGACCATTTTCATCATCAAAGGGGTGAGGAAAAGCAACGCTGCT | |
| | GAGCGGAGAGGTCCTCTGTGA | |
| IL-12ß | MCHQQLVISWFSLVFLASPLVAIWELKKDVYVVELDWYPDAPGEMVVLTCDTPEEDGITWTLD | 167 |
| | QSSEVLGSGKTLTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAERVRGDNKEYEY | |
| | SVECQEDSACPAAEESLPIEVMVDAVHKLKYENYTSSFFIRDIIKPDPPKNLQLKPLKNSRQV | |
| | EVSWEYPDTWSTPHSYFSLTFCVQVQGKSKREKKDRVFTDKTSATVICRKNASISVRAQDRYY | |
| | SSSWSEWASVPCS | |
| | ATGTGTCACCAGCAGTTGGTCATCTCTTGGTTTTCCCTGGTTTTTCTGGCATCTCCCCTCGTG | 168 |
| | GCCATATGGGAACTGAAGAAAGATGTTTATGTCGTAGAATTGGATTGGTATCCGGATGCCCCT | |
| | GGAGAAATGGTGGTCCTCACCTGTGACACCCCTGAAGAAGATGGTATCACCTGGACCTTGGAC | |
| | CAGAGCAGTGAGGTCTTAGGCTCTGGCAAAACCCTGACCATCCAAGTCAAAGAGTTTGGAGAT | |
| | GCTGGCCAGTACACCTGTCACAAAGGAGGCGAGGTTCTAAGCCATTCGCTCCTGCTGCTTCAC | |
| | AAAAAGGAAGATGGAATTTGGTCCACTGATATTTTAAAGGACCAGAAAGAACCCAAAAATAAG | |
| | ACCTTTCTAAGATGCGAGGCCAAGAATTATTCTGGACGTTTCACCTGCTGGTGGCTGACGACA | |
| | ATCAGTACTGATTTGACATTCAGTGTCAAAAGCAGCAGAGGCTCTTCTGACCCCCAAGGGGTG | |
| | ACGTGCGGAGCTGCTACACTCTCTGCAGAGAGAGTCAGAGGGGACAACAAGGAGTATGAGTAC | |
| | TCAGTGGAGTGCCAGGAGGACAGTGCCTGCCCAGCTGCTGAGGAGAGTCTGCCCATTGAGGTC | |
| | ATGGTGGATGCCGTTCACAAGCTCAAGTATGAAAACTACACCAGCAGCTTCTTCATCAGGGAC | |
| | ATCATCAAACCTGACCCACCCAAGAACTTGCAGCTGAAGCCATTAAAGAATTCTCGGCAGGTG | |
| | GAGGTCAGCTGGGAGTACCCTGACACCTGGAGTACTCCACATTCCTACTTCTCCCTGACATTC | |
| | TGCGTTCAGGTCCAGGGCAAGAGCAAGAGAGAAAAGAAAGATAGAGTCTTCACGGACAAGACC | |
| | TCAGCCACGGTCATCTGCCGCAAAAATGCCAGCATTAGCGTGCGGGCCCAGGACCGCTACTAT | |
| | AGCTCATCTTGGAGCGAATGGGCATCTGTGCCCTGCAGT | |
| Linker | GGGGSGGGGSGGGGS | 169 |
| | GGAGGAGGCGGGTCTGGCGGCGGAGGGAGCGGTGGCGGTGGGTCC | 170 |
| IL-12α | RNLPVATPDPGMFPCLHHSQNLLRAVSNMLQKARQTLEFYPCTSEEIDHEDITKDKTSTVEAC | 171 |
| (no signal | LPLELTKNESCLNSRETSFITNGSCLASRKTSFMMALCLSSIYEDLKMYQVEFKTMNAKLEMD | |
| sequence) | PKRQIFLDQNMLAVIDELMQALNFNSETVPQKSSLEEPDFYKTKIKLCILLHAFRIRAVTIDR | |
| | VMSYLNAS | |
| | AGAAACCTCCCCGTGGCCACTCCAGACCCAGGAATGTTCCCATGCCTTCACCACTCCCAAAAC | 172 |
| | CTGCTGAGGGCCGTCAGCAACATGCTCCAGAAGGCCAGACAAACTCTAGAATTTTACCCTTGC | |
| | ACTTCTGAAGAGATTGATCATGAAGATATCACAAAAGATAAAACCAGCACAGTGGAGGCCTGT | |
| | TTACCATTGGAATTAACCAAGAATGAGAGTTGCCTAAATTCCAGAGAGACCTCTTTCATAACT | |
| | AATGGGAGTTGCCTGGCCTCCAGAAAGACCTCTTTTATGATGGCCCTGTGCCTTAGTAGTATT | |
| | TATGAAGACTTGAAGATGTACCAGGTGGAGTTCAAGACCATGAATGCAAAGCTTCTGATGGAT | |
| | CCTAAGAGGCAGATCTTTCTAGATCAAAACATGCTGGCAGTTATTGATGAGCTGATGCAGGCC | |
| | CTGAATTTCAACAGTGAGACTGTGCCACAAAAATCCTCCCTTGAAGAACCGGATTTTTATAAA | |
| | ACTAAAATCAAGCTCTGCATACTTCTTCATGCTTTCAGAATTCGGGCAGTGACTATTGATAGA | |
| | GTGATGAGCTATCTGAATGCTTCC | |
| Linker | TSGGSGGTGGSGGTGGS | 173 |
| | ACCAGCGGAGGCTCTGGAGGGACAGGTGGGAGTGGTGGCACTGGAGGCTCC | 174 |
| MFGE8 | LDICSKNPCHNGGLCEEISQEVRGEVFPSYTCTCLKGYAGNHCETKCVEPLGLENGNIANSQI | 175 |
| (no signal | AASSVRVTFLGLQHWVPELARLNRAGMVNAWTPSSNDDNPWIQVNLLRRMWVTGVVTQGASRL | |
| sequence) | ASHEYLKAFKVAYSLNGHEFDFIHDVNKKHKEFVGNWNKNAVHVNLFETPVEAQYVRLYPTSC | |
| | HTACTLRFELLGCELNGCANPLGLKNNSIPDKQITASSSYKTWGLHLFSWNPSYARLDKQGNF | |
| | NAWVAGSYGNDQWLQVDLGSSKEVTGIITQGARNFGSVQFVASYKVAYSNDSANWTEYQDPRT | |
| | GSSKIFPGNWDNIISHKKNLFETPILARYVRILPVAWIINRIALRLELLGC | |
| | CTGGATATCTGTTCCAAAAACCCCTGCCACAACGGTGGTTTATGCGAGGAGATTTCCCAAGAA | 176 |
| | GTGCGAGGAGAAGTCTTCCCCTCGTACACCTGCACGTGCCTTAAGGGCTACGCGGGCAACCAC | |
| | TGTGAGACGAAATGTGTCGAGCCACTGGGCCTGGAGAATGGGAACATTGCCAACTCACAGATC | |
| | GCCGCCTCGTCTGTGCGTGTGACCTTCTTGGGTTTGCAGCATTGGGTCCCGGAGCTGGCCCGC | |
| | CTGAACCGCGCAGGCATGGTCAATGCCTGGACACCCAGCAGCAATGACGATAACCCCTGGATC | |
| | CAGGTGAACCTGCTGCGGAGGATGTGGGTAACAGGTGTGGTGACGCAGGGTGCCAGCCGCTTG | |
| | GCCAGTCATGAGTACCTGAAGGCCTTCAAGGTGGCCTACAGCCTTAATGGACACGAATTCGAT | |
| | TTCATCCATGATGTTAATAAAAAACACAAGGAGTTTGTGGGTAACTGGAACAAAAACGCGGTG | |
| | CATGTCAACCTGTTTGAGACCCCTGTGGAGGCTCAGTACGTGAGATTGTACCCCACGAGCTGC | |
| | CACACGGCCTGCACTCTGCGCTTTGAGCTACTGGGCTGTGAGCTGAACGGATGCGCCAATCCC | |
| | CTGGGCCTGAAGAATAACAGCATCCCTGACAAGCAGATCACGGCCTCCAGCAGCTACAAGACC | |
| | TGGGGCTTGCATCTCTTCAGCTGGAACCCCTCCTATGCACGGCTGGACAAGCAGGGCAACTTC | |
| | AACGCCTGGGTTGCGGGGAGCTACGGTAACGATCAGTGGCTGCAGGTGGACCTGGGCTCCTCG | |
| | AAGGAGGTGACAGGCATCATCACCCAGGGGGCCCGTAACTTTGGCTCTGTCCAGTTTGTGGCA | |
| | TCCTACAAGGTTGCCTACAGTAATGACAGTGCGAACTGGACTGAGTACCAGGACCCCAGGACT | |
| | GGCAGCAGTAAGATCTTCCCTGGCAACTGGGACAACCACTCCCACAAGAAGAACTTGTTTGAG | |
| | ACGCCCATCCTGGCTCGCTATGTGCGCATCCTGCCTGTAGCCTGGCACAACCGCATCGCCCTG | |
| | CGCCTGGAGCTGCTGGGCTGTTGA | |
| IL-12ß- | MCHQQLVISWFSLVFLASPLVAIWELKKDVYVVELDWYPDAPGEMVVLTCDTPEEDGITWTLD | 177 |
| IL12α-MFGE8 | QSSEVLGSGKTLTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAERVRGDNKEYEY | |
| | SVECQEDSACPAAEESLPIEVMVDAVHKLKYENYTSSFFIRDIIKPDPPKNLQLKPLKNSRQV | |
| | EVSWEYPDTWSTPHSYFSLTFCVQVQGKSKREKKDRVFTDKTSATVICRKNASISVRAQDRYY | |
| | SSSWSEWASVPCSGGGGSGGGGSGGGGSRNLPVATPDPGMFPCLHHSQNLLRAVSNMLQKARQ | |
| | TLEFYPCTSEEIDHEDITKDKTSTVEACLPLELTKNESCLNSRETSFITNGSCLASRKTSFMM | |
| | ALCLSSIYEDLKMYQVEFKTMNAKLLMDPKRQIFLDQNMLAVIDELMQALNFNSETVPQKSSL | |
| | EEPDFYKTKIKLCILLHAFRIRAVTIDRVMSYLNASTSGGSGGTGGSGGTGGSLDICSKNPCH | |
| | NGGLCEEISQEVRGEVFPSYTCTCLKGYAGNHCETKCVEPLGLENGNIANSQIAASSVRVTFL | |
| | GLQHWVPELARLNRAGMVNAWTPSSNDDNPWIQVNLLRRMWVTGVVTQGASRLASHEYLKAFK | |
| | VAYSLNGHEFDFIHDVNKKHKEFVGNWNKNAVHVNLFETPVEAQYVRLYPTSCHTACTLRFEL | |
| | LGCELNGCANPLGLKNNSIPDKQITASSSYKTWGLHLFSWNPSYARLDKQGNFNAWVAGSYGN | |
| | DQWLQVDLGSSKEVTGIITQGARNFGSVQFVASYKVAYSNDSANWTEYQDPRTGSSKIFPGNW | |
| | DNHSHKKNLFETPILARYVRILPVAWHNRIALRLELLGC | |
| | ATGTGTCACCAGCAGTTGGTCATCTCTTGGTTTTCCCTGGTTTTTCTGGCATCTCCCCTCGTG | 178 |
| | GCCATATGGGAACTGAAGAAAGATGTTTATGTCGTAGAATTGGATTGGTATCCGGATGCCCCT | |
| | GGAGAAATGGTGGTCCTCACCTGTGACACCCCTGAAGAAGATGGTATCACCTGGACCTTGGAC | |
| | CAGAGCAGTGAGGTCTTAGGCTCTGGCAAAACCCTGACCATCCAAGTCAAAGAGTTTGGAGAT | |
| | GCTGGCCAGTACACCTGTCACAAAGGAGGCGAGGTTCTAAGCCATTCGCTCCTGCTGCTTCAC | |
| | AAAAAGGAAGATGGAATTTGGTCCACTGATATTTTAAAGGACCAGAAAGAACCCAAAAATAAG | |
| | ACCTTTCTAAGATGCGAGGCCAAGAATTATTCTGGACGTTTCACCTGCTGGTGGCTGACGACA | |
| | ATCAGTACTGATTTGACATTCAGTGTCAAAAGCAGCAGAGGCTCTTCTGACCCCCAAGGGGTG | |
| | ACGTGCGGAGCTGCTACACTCTCTGCAGAGAGAGTCAGAGGGGACAACAAGGAGTATGAGTAC | |
| | TCAGTGGAGTGCCAGGAGGACAGTGCCTGCCCAGCTGCTGAGGAGAGTCTGCCCATTGAGGTC | |
| | ATGGTGGATGCCGTTCACAAGCTCAAGTATGAAAACTACACCAGCAGCTTCTTCATCAGGGAC | |
| | ATCATCAAACCTGACCCACCCAAGAACTTGCAGCTGAAGCCATTAAAGAATTCTCGGCAGGTG | |
| | GAGGTCAGCTGGGAGTACCCTGACACCTGGAGTACTCCACATTCCTACTTCTCCCTGACATTC | |
| | TGCGTTCAGGTCCAGGGCAAGAGCAAGAGAGAAAAGAAAGATAGAGTCTTCACGGACAAGACC | |
| | TCAGCCACGGTCATCTGCCGCAAAAATGCCAGCATTAGCGTGCGGGCCCAGGACCGCTACTAT | |
| | AGCTCATCTTGGAGCGAATGGGCATCTGTGCCCTGCAGTGGAGGAGGCGGGTCTGGCGGCGGA | |
| | GGGAGCGGTGGCGGTGGGTCCAGAAACCTCCCCGTGGCCACTCCAGACCCAGGAATGTTCCCA | |
| | TGCCTTCACCACTCCCAAAACCTGCTGAGGGCCGTCAGCAACATGCTCCAGAAGGCCAGACAA | |
| | ACTCTAGAATTTTACCCTTGCACTTCTGAAGAGATTGATCATGAAGATATCACAAAAGATAAA | |
| | ACCAGCACAGTGGAGGCCTGTTTACCATTGGAATTAACCAAGAATGAGAGTTGCCTAAATTCC | |
| | AGAGAGACCTCTTTCATAACTAATGGGAGTTGCCTGGCCTCCAGAAAGACCTCTTTTATGATG | |
| | GCCCTGTGCCTTAGTAGTATTTATGAAGACTTGAAGATGTACCAGGTGGAGTTCAAGACCATG | |
| | AATGCAAAGCTTCTGATGGATCCTAAGAGGCAGATCTTTCTAGATCAAAACATGCTGGCAGTT | |
| | ATTGATGAGCTGATGCAGGCCCTGAATTTCAACAGTGAGACTGTGCCACAAAAATCCTCCCTT | |
| | GAAGAACCGGATTTTTATAAAACTAAAATCAAGCTCTGCATACTTCTTCATGCTTTCAGAATT | |
| | CGGGCAGTGACTATTGATAGAGTGATGAGCTATCTGAATGCTTCCACCAGCGGAGGCTCTGGA | |
| | GGGACAGGTGGGAGTGGTGGCACTGGAGGCTCCCTGGATATCTGTTCCAAAAACCCCTGCCAC | |
| | AACGGTGGTTTATGCGAGGAGATTTCCCAAGAAGTGCGAGGAGAAGTCTTCCCCTCGTACACC | |
| | TGCACGTGCCTTAAGGGCTACGCGGGCAACCACTGTGAGACGAAATGTGTCGAGCCACTGGGC | |
| | CTGGAGAATGGGAACATTGCCAACTCACAGATCGCCGCCTCGTCTGTGCGTGTGACCTTCTTG | |
| | GGTTTGCAGCATTGGGTCCCGGAGCTGGCCCGCCTGAACCGCGCAGGCATGGTCAATGCCTGG | |
| | ACACCCAGCAGCAATGACGATAACCCCTGGATCCAGGTGAACCTGCTGCGGAGGATGTGGGTA | |
| | ACAGGTGTGGTGACGCAGGGTGCCAGCCGCTTGGCCAGTCATGAGTACCTGAAGGCCTTCAAG | |
| | GTGGCCTACAGCCTTAATGGACACGAATTCGATTTCATCCATGATGTTAATAAAAAACACAAG | |
| | GAGTTTGTGGGTAACTGGAACAAAAACGCGGTGCATGTCAACCTGTTTGAGACCCCTGTGGAG | |
| | GCTCAGTACGTGAGATTGTACCCCACGAGCTGCCACACGGCCTGCACTCTGCGCTTTGAGCTA | |
| | CTGGGCTGTGAGCTGAACGGATGCGCCAATCCCCTGGGCCTGAAGAATAACAGCATCCCTGAC | |
| | AAGCAGATCACGGCCTCCAGCAGCTACAAGACCTGGGGCTTGCATCTCTTCAGCTGGAACCCC | |
| | TCCTATGCACGGCTGGACAAGCAGGGCAACTTCAACGCCTGGGTTGCGGGGAGCTACGGTAAC | |
| | GATCAGTGGCTGCAGGTGGACCTGGGCTCCTCGAAGGAGGTGACAGGCATCATCACCCAGGGG | |
| | GCCCGTAACTTTGGCTCTGTCCAGTTTGTGGCATCCTACAAGGTTGCCTACAGTAATGACAGT | |
| | GCGAACTGGACTGAGTACCAGGACCCCAGGACTGGCAGCAGTAAGATCTTCCCTGGCAACTGG | |
| | GACAACCACTCCCACAAGAAGAACTTGTTTGAGACGCCCATCCTGGCTCGCTATGTGCGCATC | |
| | CTGCCTGTAGCCTGGCACAACCGCATCGCCCTGCGCCTGGAGCTGCTGGGCTGTTGA | |
| TCRß chain | MGSRLLCWVLLCLLGAGPVNAGVTQTPKFRILKIGQSMTLQCTQDMNHNYMYWYRQDPGMGLK | 179 |
| | LIYYSVGAGITDKGEVPNGYNVSRSTTEDFPLRLELAAPSQTSVYFCASTYHGTGYFGEGSWL | |
| | TVVEDLNKVFPPEVAVFEPSEAEISHTQKATLVCLATGFFPDHVELSWWVNGKEVHSGVCTDP | |
| | QPLKEQPALNDSRYALSSRLRVSATFWQNPRNHFRCQVQFYGLSENDEWTQDRAKPVTQIVSA | |
| | EAWGRAD | |
| | ATGGGCAGCAGATTGCTCTGTTGGGTGCTGCTCTGTTTGCTTGGTGCCGGTCCAGTCAACGCT | 180 |
| | GGAGTAACCCAGACTCCTAAGTTCCGCATTCTGAAGATAGGCCAATCCATGACCCTGCAGTGT | |
| | ACCCAGGACATGAACCACAACTACATGTACTGGTATCGGCAAGATCCAGGCATGGGTCTGAAG | |
| | CTGATCTACTATAGCGTTGGTGCAGGGATTACGGACAAAGGCGAGGTGCCCAATGGCTACAAC | |
| | GTGAGTCGTTCCACTACCGAGGACTTTCCCCTCAGATTGGAACTGGCAGCTCCATCACAGACA | |
| | TCCGTCTACTTCTGCGCATCTACATACCATGGAACCGGGTATTTCGGCGAAGGTAGCTGGCTT | |
| | ACCGTGGTTGAGGACCTCAACAAGGTGTTTCCGCCTGAGGTAGCCGTCTTTGAGCCGTCTGAA | |
| | GCGGAGATCAGCCATACTCAGAAAGCCACACTGGTGTGCTTGGCAACTGGGTTCTTTCCCGAT | |
| | CATGTGGAGCTCTCTTGGTGGGTCAATGGGAAGGAGGTGCACAGTGGAGTGTGCACTGATCCA | |
| | CAACCTCTCAAAGAGCAGCCAGCGCTGAATGATAGCCGGTATGCCCTGTCAAGCAGGCTTAGA | |
| | GTTTCAGCCACATTCTGGCAGAATCCTCGCAACCACTTTAGGTGTCAGGTCCAGTTCTATGGC | |
| | CTGTCCGAAAATGACGAATGGACGCAAGACCGAGCCAAACCCGTGACACAGATCGTCAGTGCC | |
| | GAAGCTTGGGGAAGAGCTGAT | |
| P2A | GSGATNFSLLKQAGDVEENPGP | 181 |
| | GGATCTGGAGCCACGAACTTCTCTCTGTTAAAGCAAGCAGGAGACGTGGAAGAAAACCCCGGT | 182 |
| | CCT | |
| TCRα chain | MEKMLECAFIVLWLQLGWLSGIQVEQSPPDLILQEGANSTLRCNFSDSVNNLQWFHQNPWGQL | 183 |
| | INLFYIPSGTKQNGRLSATTVATERYSLLYISSSQTTDSGVYFCAALIQGAQKLVFGQGTRLT | |
| | INPNIQNPDPAVYQLRDSKSSDKSVCLFTDFDSQTNVSQSKDSDVYITDKCVLDMRSMDFKSN | |
| | SAVAWSNKSDFACANAFNNSIIPEDTFFPSPESS | |
| | ATGGAGAAAATGCTTGAGTGTGCCTTCATTGTGCTGTGGCTGCAACTTGGGTGGCTGTCAGGG | 184 |
| | ATACAAGTGGAACAGAGTCCACCCGATTTGATCCTCCAGGAAGGAGCCAATTCCACCCTTCGA | |
| | TGCAACTTTAGCGATTCAGTGAACAACCTCCAGTGGTTCCACCAGAATCCCTGGGGTCAGCTG | |
| | ATCAATCTGTTCTACATTCCTTCTGGCACCAAGCAGAATGGGCGTCTTTCCGCCACAACTGTC | |
| | GCAACAGAGCGCTATTCTCTGCTGTACATCAGCAGTTCCCAGACTACCGATTCTGGCGTCTAC | |
| | TTTTGTGCCGCATTGATTCAAGGAGCACAGAAGCTCGTGTTTGGGCAAGGCACAAGGCTGACC | |
| | ATTAACCCCAACATACAGAACCCAGATCCTGCTGTGTATCAGCTGAGAGACAGCAAAAGTAGC | |
| | GATAAGTCAGTATGCCTGTTCACAGACTTTGACTCTCAGACTAATGTGAGCCAATCCAAAGAT | |
| | TCCGACGTCTATATCACCGACAAATGCGTTCTGGACATGCGGTCAATGGACTTCAAGAGCAAT | |
| | TCTGCTGTTGCTTGGAGTAACAAGTCCGACTTTGCCTGTGCCAATGCGTTCAACAATAGCATC | |
| | ATTCCGGAGGATACGTTCTTTCCAAGCCCTGAGTCATCT | |
| P2A | GSGATNFSLLKQAGDVEENPGP | 185 |
| | GGATCTGGAGCCACGAACTTCTCTCTGTTAAAGCAAGCAGGAGACGTGGAAGAAAACCCCGGT | 186 |
| | CCT | |
| Venus | MVSKGEELFTGVVPILVELDGDVNGHKFSVSGEGEGDATYGKLTLKLICTTGKLPVPWPTLVT | 187 |
| | TLGYGLQCFARYPDHMKQHDFFKSAMPEGYVQERTIFFKDDGNYKTRAEVKFEGDTLVNRIEL | |
| | KGIDFKEDGNILGHKLEYNYNSHNVYITADKQKNGIKANFKIRHNIEDGGVQLADHYQQNTPI | |
| | GDGPVLLPDNHYLSYQSALSKDPNEKRDHMVLLEFVTAAGITLGMDELYK | |
| | ATGGTGAGCAAGGGCGAGGAGCTGTTCACCGGGGTGGTGCCCATCCTGGTCGAGCTGGACGGC | 188 |
| | GACGTAAACGGCCACAAGTTCAGCGTGTCCGGCGAGGGCGAGGGCGATGCCACCTACGGCAAG | |
| | CTGACCCTGAAGCTGATCTGCACCACCGGCAAGCTGCCCGTGCCCTGGCCCACCCTCGTGACC | |
| | ACCCTGGGCTACGGCCTGCAGTGCTTCGCCCGCTACCCCGACCACATGAAGCAGCACGACTTC | |
| | TTCAAGTCCGCCATGCCCGAAGGCTACGTCCAGGAGCGCACCATCTTCTTCAAGGACGACGGC | |
| | AACTACAAGACCCGCGCCGAGGTGAAGTTCGAGGGCGACACCCTGGTGAACCGCATCGAGCTG | |
| | AAGGGCATCGACTTCAAGGAGGACGGCAACATCCTGGGGCACAAGCTGGAGTACAACTACAAC | |
| | AGCCACAACGTCTATATCACCGCCGACAAGCAGAAGAACGGCATCAAGGCCAACTTCAAGATC | |
| | CGCCACAACATCGAGGACGGCGGCGTGCAGCTCGCCGACCACTACCAGCAGAACACCCCCATC | |
| | GGCGACGGCCCCGTGCTGCTGCCCGACAACCACTACCTGAGCTACCAGTCCGCCCTGAGCAAA | |
| | GACCCCAACGAGAAGCGCGATCACATGGTCCTGCTGGAGTTCGTGACCGCCGCCGGGATCACT | |
| | CTCGGCATGGACGAGCTGTACAAGTAA | |
| Fusion protein | MGSRLLCWVLLCLLGAGPVNAGVTQTPKFRILKIGQSMTLQCTQDMNHNYMYWYRQDPGMGLK | 189 |
| of TPI-1 | LIYYSVGAGITDKGEVPNGYNVSRSTTEDFPLRLELAAPSQTSVYFCASTYHGTGYFGEGSWL | |
| peptide- | TVVEDLNKVFPPEVAVFEPSEAEISHTQKATLVCLATGFFPDHVELSWWVNGKEVHSGVCTDP | |
| specific TCR | QPLKEQPALNDSRYALSSRLRVSATFWQNPRNHFRCQVQFYGLSENDEWTQDRAKPVTQIVSA | |
| and Venus | EAWGRADGSGATNFSLLKQAGDVEENPGPMEKMLECAFIVLWLQLGWLSGIQVEQSPPDLILQ | |
| | EGANSTLRCNFSDSVNNLQWFHQNPWGQLINLFYIPSGTKQNGRLSATTVATERYSLLYISSS | |
| | QTTDSGVYFCAALIQGAQKLVFGQGTRLTINPNIQNPDPAVYQLRDSKSSDKSVCLFTDFDSQ | |
| | TNVSQSKDSDVYITDKCVLDMRSMDFKSNSAVAWSNKSDFACANAFNNSIIPEDTFFPSPESS | |
| | GSGATNFSLLKQAGDVEENPGPMVSKGEELFTGVVPILVELDGDVNGHKFSVSGEGEGDATYG | |
| | KLTLKLICTTGKLPVPWPTLVTTLGYGLQCFARYPDHMKQHDFFKSAMPEGYVQERTIFFKDD | |
| | GNYKTRAEVKFEGDTLVNRIELKGIDFKEDGNILGHKLEYNYNSHNVYITADKQKNGIKANFK | |
| | IRHNIEDGGVQLADHYQQNTPIGDGPVLLPDNHYLSYQSALSKDPNEKRDHMVLLEFVTAAGI | |
| | TLGMDELYK | |
| | ATGGGCAGCAGATTGCTCTGTTGGGTGCTGCTCTGTTTGCTTGGTGCCGGTCCAGTCAACGCT | 190 |
| | GGAGTAACCCAGACTCCTAAGTTCCGCATTCTGAAGATAGGCCAATCCATGACCCTGCAGTGT | |
| | ACCCAGGACATGAACCACAACTACATGTACTGGTATCGGCAAGATCCAGGCATGGGTCTGAAG | |
| | CTGATCTACTATAGCGTTGGTGCAGGGATTACGGACAAAGGCGAGGTGCCCAATGGCTACAAC | |
| | GTGAGTCGTTCCACTACCGAGGACTTTCCCCTCAGATTGGAACTGGCAGCTCCATCACAGACA | |
| | TCCGTCTACTTCTGCGCATCTACATACCATGGAACCGGGTATTTCGGCGAAGGTAGCTGGCTT | |
| | ACCGTGGTTGAGGACCTCAACAAGGTGTTTCCGCCTGAGGTAGCCGTCTTTGAGCCGTCTGAA | |
| | GCGGAGATCAGCCATACTCAGAAAGCCACACTGGTGTGCTTGGCAACTGGGTTCTTTCCCGAT | |
| | CATGTGGAGCTCTCTTGGTGGGTCAATGGGAAGGAGGTGCACAGTGGAGTGTGCACTGATCCA | |
| | CAACCTCTCAAAGAGCAGCCAGCGCTGAATGATAGCCGGTATGCCCTGTCAAGCAGGCTTAGA | |
| | GTTTCAGCCACATTCTGGCAGAATCCTCGCAACCACTTTAGGTGTCAGGTCCAGTTCTATGGC | |
| | CTGTCCGAAAATGACGAATGGACGCAAGACCGAGCCAAACCCGTGACACAGATCGTCAGTGCC | |
| | GAAGCTTGGGGAAGAGCTGATGGATCTGGAGCCACGAACTTCTCTCTGTTAAAGCAAGCAGGA | |
| | GACGTGGAAGAAAACCCCGGTCCTATGGAGAAAATGCTTGAGTGTGCCTTCATTGTGCTGTGG | |
| | CTGCAACTTGGGTGGCTGTCAGGGATACAAGTGGAACAGAGTCCACCCGATTTGATCCTCCAG | |
| | GAAGGAGCCAATTCCACCCTTCGATGCAACTTTAGCGATTCAGTGAACAACCTCCAGTGGTTC | |
| | CACCAGAATCCCTGGGGTCAGCTGATCAATCTGTTCTACATTCCTTCTGGCACCAAGCAGAAT | |
| | GGGCGTCTTTCCGCCACAACTGTCGCAACAGAGCGCTATTCTCTGCTGTACATCAGCAGTTCC | |
| | CAGACTACCGATTCTGGCGTCTACTTTTGTGCCGCATTGATTCAAGGAGCACAGAAGCTCGTG | |
| | TTTGGGCAAGGCACAAGGCTGACCATTAACCCCAACATACAGAACCCAGATCCTGCTGTGTAT | |
| | CAGCTGAGAGACAGCAAAAGTAGCGATAAGTCAGTATGCCTGTTCACAGACTTTGACTCTCAG | |
| | ACTAATGTGAGCCAATCCAAAGATTCCGACGTCTATATCACCGACAAATGCGTTCTGGACATG | |
| | CGGTCAATGGACTTCAAGAGCAATTCTGCTGTTGCTTGGAGTAACAAGTCCGACTTTGCCTGT | |
| | GCCAATGCGTTCAACAATAGCATCATTCCGGAGGATACGTTCTTTCCAAGCCCTGAGTCATCT | |
| | GGATCTGGAGCCACGAACTTCTCTCTGTTAAAGCAAGCAGGAGACGTGGAAGAAAACCCCGGT | |
| | CCTATGGTGAGCAAGGGCGAGGAGCTGTTCACCGGGGTGGTGCCCATCCTGGTCGAGCTGGAC | |
| | GGCGACGTAAACGGCCACAAGTTCAGCGTGTCCGGCGAGGGCGAGGGCGATGCCACCTACGGC | |
| | AAGCTGACCCTGAAGCTGATCTGCACCACCGGCAAGCTGCCCGTGCCCTGGCCCACCCTCGTG | |
| | ACCACCCTGGGCTACGGCCTGCAGTGCTTCGCCCGCTACCCCGACCACATGAAGCAGCACGAC | |
| | TTCTTCAAGTCCGCCATGCCCGAAGGCTACGTCCAGGAGCGCACCATCTTCTTCAAGGACGAC | |
| | GGCAACTACAAGACCCGCGCCGAGGTGAAGTTCGAGGGCGACACCCTGGTGAACCGCATCGAG | |
| | CTGAAGGGCATCGACTTCAAGGAGGACGGCAACATCCTGGGGCACAAGCTGGAGTACAACTAC | |
| | AACAGCCACAACGTCTATATCACCGCCGACAAGCAGAAGAACGGCATCAAGGCCAACTTCAAG | |
| | ATCCGCCACAACATCGAGGACGGCGGCGTGCAGCTCGCCGACCACTACCAGCAGAACACCCCC | |
| | ATCGGCGACGGCCCCGTGCTGCTGCCCGACAACCACTACCTGAGCTACCAGTCCGCCCTGAGC | |
| | AAAGACCCCAACGAGAAGCGCGATCACATGGTCCTGCTGGAGTTCGTGACCGCCGCCGGGATC | |
| | ACTCTCGGCATGGACGAGCTGTACAAGTAA | |

TABLE 16-1

| | | SEQ |
|---|---|---|
| | Sequence | ID NO: |
| sc-Trimer | MARSVTLVFLVLVSLTGLYASIINFEKLGGGASGGGGSGGGGSIQKTPQIQVYSRHPPENGKP | 191 |
| | NILNCYVTQFHPPHIEIQMLKNGKKIPKVEMSDMSFSKDWSFYILAHTEFTPTETDTYACRVK | |
| | HASMAEPKTVYWDRDMGGGGSGGGGSGGGGSGGGGSGPHSLRYFVTAVSRPGLGEPRYMEVGY | |
| | VDDTEFVRFDSDAENPRYEPRARWMEQEGPEYWERETQKAKGNEQSFRVDLRTLLGYYNQSKG | |
| | GSHTIQVISGCEVGSDGRLLRGYQQYAYDGCDYIALNEDLKTWTAADMAALITKHKWEQAGEA | |
| | ERLRAYLEGTCVEWLRRYLKNGNATLLRTDSPKAHVTHHSRPEDKVTLRCWALGFYPADITLT | |
| | WQLNGEELIQDMELVERPAGDGTFQKWASVVVPLGKEQYYTCHVYHQGLPEPLTLRWEPPPST | |
| | VSNMATVAVLVVLGAAIVTGAVVAFVMKMRRRNTGGKGGDYALAPGSQTSDLSLPDCKVMVHD | |
| | PHSLA | |
| | ATGGCTCGCTCGGTGACCCTGGTCTTTCTGGTGCTTGTCTCACTGACCGGCCTGTATGCTTCC | 192 |
| | ATTATAAATTTTGAAAAGTTGGGCGGAGGTGCCTCTGGCGGTGGGGGCAGCGGTGGAGGGGGC | |
| | AGTATCCAGAAAACCCCTCAAATTCAAGTATACTCACGCCACCCACCGGAGAATGGGAAGCCG | |
| | AACATACTGAACTGCTACGTAACACAGTTCCACCCGCCTCACATTGAAATCCAAATGCTGAAG | |
| | AACGGGAAAAAAATTCCTAAAGTAGAGATGTCAGATATGTCCTTCAGCAAGGACTGGTCTTTC | |
| | TATATCCTGGCTCACACTGAATTCACCCCCACTGAGACTGATACATACGCCTGCAGAGTTAAG | |
| | CATGCCAGTATGGCCGAGCCCAAGACCGTCTACTGGGATCGAGACATGGGGGGGGGAGGCTCC | |
| | GGTGGAGGGGGGTCTGGAGGGGGGGGGTCTGGTGGAGGCGGAAGTGGCCCACACTCGCTGAGG | |
| | TATTTCGTCACCGCCGTGTCCCGGCCCGGCCTCGGGGAGCCCCGGTACATGGAAGTCGGCTAC | |
| | GTGGACGACACGGAGTTCGTGCGCTTCGACAGCGACGCGGAGAATCCGAGATATGAGCCGCGG | |
| | GCGCGGTGGATGGAGCAGGAGGGGCCCGAGTATTGGGAGCGGGAGACACAGAAAGCCAAGGGC | |
| | AATGAGCAGAGTTTCCGAGTGGACCTGAGGACCCTGCTCGGCTACTACAACCAGAGCAAGGGC | |
| | GGCTCTCACACTATTCAGGTGATCTCTGGCTGTGAAGTGGGGTCCGACGGGCGACTCCTCCGC | |
| | GGGTACCAGCAGTACGCCTACGACGGCTGCGATTACATCGCCCTGAACGAAGACCTGAAAACG | |
| | TGGACGGCGGCGGACATGGCGGCGCTGATCACCAAACACAAGTGGGAGCAGGCTGGTGAAGCA | |
| | GAGAGACTCAGGGCCTACCTGGAGGGCACGTGCGTGGAGTGGCTCCGCAGATACCTGAAGAAC | |
| | GGGAACGCGACGCTGCTGCGCACAGATTCCCCAAAGGCCCATGTGACCCATCACAGCAGACCT | |
| | GAAGATAAAGTCACCCTGAGGTGCTGGGCCCTGGGCTTCTACCCTGCTGACATCACCCTGACC | |
| | TGGCAGTTGAATGGGGAGGAGCTGATCCAGGACATGGAGCTTGTGGAGACCAGGCCTGCAGGG | |
| | GATGGAACCTTCCAGAAGTGGGCATCTGTGGTGGTGCCTCTTGGGAAGGAGCAGTATTACACA | |
| | TGCCATGTGTACCATCAGGGGCTGCCTGAGCCCCTCACCCTGAGATGGGAGCCTCCTCCATCC | |
| | ACTGTCTCCAACATGGCGACCGTTGCTGTTCTGGTTGTCCTTGGAGCTGCAATAGTCACTGGA | |
| | GCTGTGGTGGCTTTTGTGATGAAGATGAGAAGGAGAAACACAGGTGGAAAAGGAGGGGACTAT | |
| | GCTCTGGCTCCAGGCTCCCAGACCTCTGATCTGTCTCTCCCAGATTGTAAAGTGATGGTTCAT | |
| | GACCCTCATTCTCTAGCG | |
| T2A | GSGEGRGSLLTCGDVEENPGP | 193 |
| | GGCTCCGGCGAGGGCAGGGGAAGTCTTCTAACATGCGGGGACGTGGAGGAAAATCCCGGCCCA | 194 |
| IL-2 | MYSMQLASCVTLTLVLLVNSAPTSSSTSSSTAEAQQQQQQQQQQQQHLEQLLMDLQELLSRME | 195 |
| | NYRNLKLPRMLTFKFYLPKQATELKDLQCLEDELGPLRHVLDLTQSKSFQLEDAENFISNIRV | |
| | TVVKLKGSDNTFECQFDDESATVVDFLRRWIAFCQSIISTSPQ | |
| | ATGTACTCAATGCAGCTGGCTAGTTGTGTGACCCTGACCCTCGTGCTGCTCGTGAACAGCGCC | 196 |
| | CCAACCTCAAGCTCTACCTCCAGTAGCACAGCCGAAGCTCAGCAGCAGCAGCAACAGCAGCAG | |
| | CAGCAGCAGCAGCACCTGGAGCAGCTGCTGATGGACCTGCAGGAGCTGCTGAGCCGGATGGAG | |
| | AACTACAGGAACCTGAAGCTGCCTAGGATGCTGACATTCAAGTTCTACCTGCCAAAGCAGGCC | |
| | ACCGAGCTGAAGGACCTGCAGTGTCTGGAGGACGAGCTGGGCCCCCTGCGCCACGTGCTCGAC | |
| | CTGACACAGTCCAAGTCCTTCCAGCTGGAGGACGCAGAGAACTTCATCTCCAACATCAGAGTG | |
| | ACCGTGGTGAAACTGAAGGGCTCTGACAACACCTTTGAGTGTCAGTTCGACGACGAGAGTGCC | |
| | ACTGTGGTGGATTTCCTGAGGCGGTGGATCGCTTTCTGCCAGAGCATTATCTCTACCAGCCCA | |
| | CAG | |
| Linker | GGGGS | 197 |
| | GGTGGCGGCGGAAGC | 198 |
| CD8 | VISNSVMYFSSVVPVLQKVNSTTTKPVLRTPSPVHPTGTSQPQRPEDCRPRGSVKGTGLDFAC | 199 |
| (partial | DIYIWAPLAGICVALLLSLIITLICYHRSRKRVCKCPRPLVRQEGKPRPSEKIV | |
| sequence) | GTCATCAGCAACAGCGTGATGTATTTCTCCTCTGTGGTGCCTGTGCTGCAGAAGGTGAACAGC | 200 |
| | ACCACCACCAAGCCTGTGCTGAGGACTCCAAGCCCCGTGCACCCCACTGGTACTAGCCAGCCT | |
| | CAGCGCCCCGAGGACTGTAGACCTAGAGGATCTGTGAAAGGAACAGGCCTGGACTTTGCATGT | |
| | GACATCTATATTTGGGCACCACTTGCCGGCATTTGCGTGGCCCTGCTGCTGTCCCTGATCATC | |
| | ACACTGATCTGCTACCACAGGAGCCGAAAGCGTGTTTGCAAATGTCCCAGGCCGCTAGTCAGA | |
| | CAGGAAGGCAAGCCCAGACCTTCAGAGAAAATTGTG | |
| P2A | GSGATNFSLLKQAGDVEENPGP | 202 |
| | GGTTCAGGGGCTACCAACTTTTCCCTCCTTAAGCAGGCCGGAGATGTCGAGGAGAATCCTGGC | 202 |
| | CCT | |
| CD80 | MACNCQLMQDTPLLKFPCPRLILLFVLLIRLSQVSSDVDEQLSKSVKDKVLLPCRYNSPHEDE | 203 |
| | SEDRIYWQKHDKVVLSVIAGKLKVWPEYKNRTLYDNTTYSLIILGLVLSDRGTYSCVVQKKER | |
| | GTYEVKHLALVKLSIKADFSTPNITESGNPSADTKRITCFASGGFPKPRFSWLENGRELPGIN | |
| | TTISQDPESELYTISSQLDFNTTRNHTIKCLIKYGDAHVSEDFTWEKPPEDPPDSKNTLVLFG | |
| | AGFGAVITVVVIVVIIKCFCKHRSCFRRNEASRETNNSLTFGPEEALAEQTVFL | |
| | ATGGCGTGTAATTGCCAGCTCATGCAGGACACACCTCTGTTGAAATTCCCGTGTCCAAGGCTT | 204 |
| | ATCCTGCTGTTCGTGCTCTTGATACGACTCAGCCAGGTGTCAAGCGACGTTGATGAGCAGCTG | |
| | TCTAAGAGCGTGAAGGATAAGGTGTTGCTGCCCTGTCGGTACAATAGCCCACATGAGGACGAG | |
| | TCAGAGGATCGCATCTATTGGCAGAAACACGACAAAGTGGTGCTGAGCGTGATCGCTGGCAAG | |
| | CTGAAAGTTTGGCCCGAGTACAAGAACCGGACACTGTACGACAATACCACATACTCCCTGATT | |
| | ATTCTGGGGCTCGTGCTCTCCGATAGAGGCACTTATAGCTGCGTTGTACAGAAGAAGGAAAGA | |
| | GGGACTTATGAGGTTAAACACCTTGCTCTGGTGAAGCTGAGTATCAAGGCTGACTTCTCCACG | |
| | CCAAATATAACCGAATCAGGAAATCCTAGTGCCGATACTAAACGCATCACTTGTTTTGCCAGT | |
| | GGTGGGTTCCCCAAACCGAGATTCTCTTGGCTGGAAAACGGAAGGGAGTTGCCCGGCATTAAC | |
| | ACCACCATTTCTCAAGACCCCGAATCCGAGCTTTACACCATTAGCAGCCAACTTGACTTCAAC | |
| | ACAACACGGAACCACACCATCAAGTGTCTGATCAAGTATGGCGACGCACATGTCAGTGAGGAT | |
| | TTCACATGGGAGAAACCACCCGAAGATCCTCCAGACTCCAAGAACACTCTCGTGCTGTTTGGT | |
| | GCAGGATTTGGAGCCGTAATAACCGTAGTGGTCATTGTCGTCATCATCAAGTGCTTCTGCAAA | |
| | CACCGTTCTTGCTTTCGACGCAATGAAGCCTCTAGGGAAACAAACAACTCTCTGACTTTTGGC | |
| | CCTGAAGAAGCCCTGGCAGAGCAAACGGTCTTTCTGTAG | |
| sc-Trimer- | MARSVTLVFLVLVSLTGLYASIINFEKLGGGASGGGGSGGGGSIQKTPQIQVYSRHPPENGKP | 205 |
| T2A-IL-2- | NILNCYVTQFHPPHIEIQMLKNGKKIPKVEMSDMSFSKDWSFYILAHTEFTPTETDTYACRVK | |
| CD8-P2A- | HASMAEPKTVYWDRDMGGGGSGGGGSGGGGSGGGGSGPHSLRYFVTAVSRPGLGEPRYMEVGY | |
| CD80 | VDDTEFVRFDSDAENPRYEPRARWMEQEGPEYWERETQKAKGNEQSFRVDLRTLLGYYNQSKG | |
| | GSHTIQVISGCEVGSDGRLLRGYQQYAYDGCDYIALNEDLKTWTAADMAALITKHKWEQAGEA | |
| | ERLRAYLEGTCVEWLRRYLKNGNATLLRTDSPKAHVTHHSRPEDKVTLRCWALGFYPADITLT | |
| | WQLNGEELIQDMELVETRPAGDGTFQKWASVVVPLGKEQYYTCHVYHQGLPEPLTLRWEPPPS | |
| | TVSNMATVAVLVVLGAAIVTGAVVAFVMKMRRRNTGGKGGDYALAPGSQTSDLSLPDCKVMVH | |
| | DPHSLAGSGEGRGSLLTCGDVEENPGPMYSMQLASCVTLTLVLLVNSAPTSSSTSSSTAEAQQ | |
| | QQQQQQQQQQHLEQLLMDLQELLSRMENYRNLKLPRMLTFKFYLPKQATELKDLQCLEDELGP | |
| | LRHVLDLTQSKSFQLEDAENFISNIRVTVVKLKGSDNTFECQFDDESATVVDFLRRWIAFCQS | |
| | IISTSPQGGGGSVISNSVMYFSSVVPVLQKVNSTTTKPVLRTPSPVHPTGTSQPQRPEDCRPR | |
| | GSVKGTGLDFACDIYIWAPLAGICVALLLSLIITLICYHRSRKRVCKCPRPLVRQEGKPRPSE | |
| | KIVGSGATNFSLLKQAGDVEENPGPMACNCQLMQDTPLLKFPCPRLILLFVLLIRLSQVSSDV | |
| | DEQLSKSVKDKVLLPCRYNSPHEDESEDRIYWQKHDKVVLSVIAGKLKVWPEYKNRTLYDNTT | |
| | YSLIILGLVLSDRGTYSCVVQKKERGTYEVKHLALVKLSIKADFSTPNITESGNPSADTKRIT | |
| | CFASGGFPKPRFSWLENGRELPGINTTISQDPESELYTISSQLDFNTTRNHTIKCLIKYGDAH | |
| | VSEDFTWEKPPEDPPDSKNTLVLFGAGFGAVITVVVIVVIIKCFCKHRSCFRRNEASRETNNS | |
| | LTFGPEEALAEQTVFL | |
| | ATGGCTCGCTCGGTGACCCTGGTCTTTCTGGTGCTTGTCTCACTGACCGGCCTGTATGCTTCC | 206 |
| | ATTATAAATTTTGAAAAGTTGGGCGGAGGTGCCTCTGGCGGTGGGGGCAGCGGTGGAGGGGGC | |
| | AGTATCCAGAAAACCCCTCAAATTCAAGTATACTCACGCCACCCACCGGAGAATGGGAAGCCG | |
| | AACATACTGAACTGCTACGTAACACAGTTCCACCCGCCTCACATTGAAATCCAAATGCTGAAG | |
| | AACGGGAAAAAAATTCCTAAAGTAGAGATGTCAGATATGTCCTTCAGCAAGGACTGGTCTTTC | |
| | TATATCCTGGCTCACACTGAATTCACCCCCACTGAGACTGATACATACGCCTGCAGAGTTAAG | |
| | CATGCCAGTATGGCCGAGCCCAAGACCGTCTACTGGGATCGAGACATGGGGGGGGGAGGCTCC | |
| | GGTGGAGGGGGGTCTGGAGGGGGGGGGTCTGGTGGAGGCGGAAGTGGCCCACACTCGCTGAGG | |
| | TATTTCGTCACCGCCGTGTCCCGGCCCGGCCTCGGGGAGCCCCGGTACATGGAAGTCGGCTAC | |
| | GTGGACGACACGGAGTTCGTGCGCTTCGACAGCGACGCGGAGAATCCGAGATATGAGCCGCGG | |
| | GCGCGGTGGATGGAGCAGGAGGGGCCCGAGTATTGGGAGCGGGAGACACAGAAAGCCAAGGGC | |
| | AATGAGCAGAGTTTCCGAGTGGACCTGAGGACCCTGCTCGGCTACTACAACCAGAGCAAGGGC | |
| | GGCTCTCACACTATTCAGGTGATCTCTGGCTGTGAAGTGGGGTCCGACGGGCGACTCCTCCGC | |
| | GGGTACCAGCAGTACGCCTACGACGGCTGCGATTACATCGCCCTGAACGAAGACCTGAAAACG | |
| | TGGACGGCGGCGGACATGGCGGCGCTGATCACCAAACACAAGTGGGAGCAGGCTGGTGAAGCA | |
| | GAGAGACTCAGGGCCTACCTGGAGGGCACGTGCGTGGAGTGGCTCCGCAGATACCTGAAGAAC | |
| | GGGAACGCGACGCTGCTGCGCACAGATTCCCCAAAGGCCCATGTGACCCATCACAGCAGACCT | |
| | GAAGATAAAGTCACCCTGAGGTGCTGGGCCCTGGGCTTCTACCCTGCTGACATCACCCTGACC | |
| | TGGCAGTTGAATGGGGAGGAGCTGATCCAGGACATGGAGCTTGTGGAGACCAGGCCTGCAGGG | |
| | GATGGAACCTTCCAGAAGTGGGCATCTGTGGTGGTGCCTCTTGGGAAGGAGCAGTATTACACA | |
| | TGCCATGTGTACCATCAGGGGCTGCCTGAGCCCCTCACCCTGAGATGGGAGCCTCCTCCATCC | |
| | ACTGTCTCCAACATGGCGACCGTTGCTGTTCTGGTTGTCCTTGGAGCTGCAATAGTCACTGGA | |
| | GCTGTGGTGGCTTTTGTGATGAAGATGAGAAGGAGAAACACAGGTGGAAAAGGAGGGGACTAT | |
| | GCTCTGGCTCCAGGCTCCCAGACCTCTGATCTGTCTCTCCCAGATTGTAAAGTGATGGTTCAT | |
| | GACCCTCATTCTCTAGCGGGCTCCGGCGAGGGCAGGGGAAGTCTTCTAACATGCGGGGACGTG | |
| | GAGGAAAATCCCGGCCCAATGTACTCAATGCAGCTGGCTAGTTGTGTGACCCTGACCCTCGTG | |
| | CTGCTCGTGAACAGCGCCCCAACCTCAAGCTCTACCTCCAGTAGCACAGCCGAAGCTCAGCAG | |
| | CAGCAGCAACAGCAGCAGCAGCAGCAGCAGCACCTGGAGCAGCTGCTGATGGACCTGCAGGAG | |
| | CTGCTGAGCCGGATGGAGAACTACAGGAACCTGAAGCTGCCTAGGATGCTGACATTCAAGTTC | |
| | TACCTGCCAAAGCAGGCCACCGAGCTGAAGGACCTGCAGTGTCTGGAGGACGAGCTGGGCCCC | |
| | CTGCGCCACGTGCTCGACCTGACACAGTCCAAGTCCTTCCAGCTGGAGGACGCAGAGAACTTC | |
| | ATCTCCAACATCAGAGTGACCGTGGTGAAACTGAAGGGCTCTGACAACACCTTTGAGTGTCAG | |
| | TTCGACGACGAGAGTGCCACTGTGGTGGATTTCCTGAGGCGGTGGATCGCTTTCTGCCAGAGC | |
| | ATTATCTCTACCAGCCCACAGGGTGGCGGCGGAAGCGTCATCAGCAACAGCGTGATGTATTTC | |
| | TCCTCTGTGGTGCCTGTGCTGCAGAAGGTGAACAGCACCACCACCAAGCCTGTGCTGAGGACT | |
| | CCAAGCCCCGTGCACCCCACTGGTACTAGCCAGCCTCAGCGCCCCGAGGACTGTAGACCTAGA | |
| | GGATCTGTGAAAGGAACAGGCCTGGACTTTGCATGTGACATCTATATTTGGGCACCACTTGCC | |
| | GGCATTTGCGTGGCCCTGCTGCTGTCCCTGATCATCACACTGATCTGCTACCACAGGAGCCGA | |
| | AAGCGTGTTTGCAAATGTCCCAGGCCGCTAGTCAGACAGGAAGGCAAGCCCAGACCTTCAGAG | |
| | AAAATTGTGGGTTCAGGGGCTACCAACTTTTCCCTCCTTAAGCAGGCCGGAGATGTCGAGGAG | |
| | AATCCTGGCCCTATGGCGTGTAATTGCCAGCTCATGCAGGACACACCTCTGTTGAAATTCCCG | |
| | TGTCCAAGGCTTATCCTGCTGTTCGTGCTCTTGATACGACTCAGCCAGGTGTCAAGCGACGTT | |
| | GATGAGCAGCTGTCTAAGAGCGTGAAGGATAAGGTGTTGCTGCCCTGTCGGTACAATAGCCCA | |
| | CATGAGGACGAGTCAGAGGATCGCATCTATTGGCAGAAACACGACAAAGTGGTGCTGAGCGTG | |
| | ATCGCTGGCAAGCTGAAAGTTTGGCCCGAGTACAAGAACCGGACACTGTACGACAATACCACA | |
| | TACTCCCTGATTATTCTGGGGCTCGTGCTCTCCGATAGAGGCACTTATAGCTGCGTTGTACAG | |
| | AAGAAGGAAAGAGGGACTTATGAGGTTAAACACCTTGCTCTGGTGAAGCTGAGTATCAAGGCT | |
| | GACTTCTCCACGCCAAATATAACCGAATCAGGAAATCCTAGTGCCGATACTAAACGCATCACT | |
| | TGTTTTGCCAGTGGTGGGTTCCCCAAACCGAGATTCTCTTGGCTGGAAAACGGAAGGGAGTTG | |
| | CCCGGCATTAACACCACCATTTCTCAAGACCCCGAATCCGAGCTTTACACCATTAGCAGCCAA | |
| | CTTGACTTCAACACAACACGGAACCACACCATCAAGTGTCTGATCAAGTATGGCGACGCACAT | |
| | GTCAGTGAGGATTTCACATGGGAGAAACCACCCGAAGATCCTCCAGACTCCAAGAACACTCTC | |
| | GTGCTGTTTGGTGCAGGATTTGGAGCCGTAATAACCGTAGTGGTCATTGTCGTCATCATCAAG | |
| | TGCTTCTGCAAACACCGTTCTTGCTTTCGACGCAATGAAGCCTCTAGGGAAACAAACAACTCT | |
| | CTGACTTTTGGCCCTGAAGAAGCCCTGGCAGAGCAAACGGTCTTTCTGTAG | |
| CD81 | MGVEGCTKCIKYLLFVFNFVFWLAGGVILGVALWLRHDPQTTSLLYLELGNKPAPNTFYVGIY | 207 |
| | ILIAVGAVMMFVGFLGCYGAIQESQCLLGTFFTCLVILFACEVAAGIWGFVNKDQIAKDVKQF | |
| | YDQALQQAVMDDDANNAKAVVKTFHETLNCCGSNALTTLTTTILRNSLCPSGGNILTPLLQQD | |
| | CHQKIDELFSGKLYLIGIAAIVVAVIMIFEMILSMVLCCGIRNSSVY | |
| | ATGGGGGTGGAGGGCTGCACCAAATGCATCAAATACCTGCTCTTCGTCTTCAATTTCGTCTTC | 208 |
| | TGGCTGGCTGGAGGCGTGATCCTAGGTGTAGCTCTGTGGTTGCGTCATGATCCACAGACCACC | |
| | AGCCTGCTGTACCTGGAACTGGGAAACAAACCGGCACCCAACACCTTCTACGTGGGCATCTAC | |
| | ATTCTCATTGCTGTGGGAGCTGTGATGATGTTTGTAGGCTTCCTGGGGTGCTATGGGGCCATC | |
| | CAGGAGTCCCAGTGTCTGCTGGGGACGTTCTTCACCTGCCTTGTGATCCTGTTTGCCTGTGAG | |
| | GTGGCTGCAGGCATCTGGGGCTTCGTAAACAAAGACCAGATCGCCAAGGATGTGAAGCAGTTC | |
| | TATGACCAGGCCCTTCAGCAAGCTGTGATGGATGATGATGCCAACAATGCCAAGGCTGTGGTG | |
| | AAGACTTTCCATGAGACGCTCAACTGTTGTGGCTCCAACGCACTGACCACACTGACTACCACC | |
| | ATACTGAGGAACAGCCTGTGTCCCTCAGGCGGCAACATACTCACCCCCTTACTGCAGCAAGAT | |
| | TGTCATCAGAAAATCGATGAGCTCTTCTCTGGGAAGCTGTACCTCATTGGAATTGCAGCCATT | |
| | GTGGTAGCTGTCATTATGATCTTTGAGATGATTCTGAGCATGGTGCTGTGCTGTGGCATCCGG | |
| | AACAGCTCCGTGTACTGA | |
| OVA | MGSIGAASMEFCFDVFKELKVHHANENIFYCPIAIMSALAMVYLGAKDSTRTQINKVVRFDKL | 209 |
| | PGFGDSIEAQCGTSVNVHSSLRDILNQITKPNDVYSFSLASRLYAEERYPILPEYLQCVKELY | |
| | RGGLEPINFQTAADQARELINSWVESQTNGIIRNVLQPSSVDSQTAMVLVNAIVFKGLWEKTF | |
| | KDEDTQAMPFRVTEQESKPVQMMYQIGLFRVASMASEKMKILELPFASGTMSMLVLLPDEVSG | |
| | LEQLESIINFEKLTEWTSSNVMEERKIKVYLPRMKMEEKYNLTSVLMAMGITDVFSSSANLSG | |
| | ISSAESLKISQAVHAAHAEINEAGREVVGSAEAGVDAASVSEEFRADHPFLFCIKHIATNAVL | |
| | FFGRCVSP | |
| | ATGGGCTCCATCGGCGCAGCAAGCATGGAATTTTGTTTTGATGTATTCAAGGAGCTCAAAGTC | 210 |
| | CACCATGCCAATGAGAACATCTTCTACTGCCCCATTGCCATCATGTCAGCTCTAGCCATGGTA | |
| | TACCTGGGTGCAAAAGACAGCACCAGGACACAGATAAATAAGGTTGTTCGCTTTGATAAACTT | |
| | CCAGGATTCGGAGACAGTATTGAAGCTCAGTGTGGCACATCTGTAAACGTTCACTCTTCACTT | |
| | AGAGACATCCTCAACCAAATCACCAAACCAAATGATGTTTATTCGTTCAGCCTTGCCAGTAGA | |
| | CTTTATGCTGAAGAGAGATACCCAATCCTGCCAGAATACTTGCAGTGTGTGAAGGAACTGTAT | |
| | AGAGGAGGCTTGGAACCTATCAACTTTCAAACAGCTGCAGATCAAGCCAGAGAGCTCATCAAT | |
| | TCCTGGGTAGAAAGTCAGACAAATGGAATTATCAGAAATGTCCTTCAGCCAAGCTCCGTGGAT | |
| | TCTCAAACTGCAATGGTTCTGGTTAATGCCATTGTCTTCAAAGGACTGTGGGAGAAAACATTT | |
| | AAGGATGAAGACACACAAGCAATGCCTTTCAGAGTGACTGAGCAAGAAAGCAAACCTGTGCAG | |
| | ATGATGTACCAGATTGGTTTATTTAGAGTGGCATCAATGGCTTCTGAGAAAATGAAGATCCTG | |
| | GAGCTTCCATTTGCCAGTGGGACAATGAGCATGTTGGTGCTGTTGCCTGATGAAGTCTCAGGC | |
| | CTTGAGCAGCTTGAGAGTATAATCAACTTTGAAAAACTGACTGAATGGACCAGTTCTAATGTT | |
| | ATGGAAGAGAGGAAGATCAAAGTGTACTTACCTCGCATGAAGATGGAGGAAAAATACAACCTC | |
| | ACATCTGTCTTAATGGCTATGGGCATTACTGACGTGTTTAGCTCTTCAGCCAATCTGTCTGGC | |
| | ATCTCCTCAGCAGAGAGCCTGAAGATATCTCAAGCTGTCCATGCAGCACATGCAGAAATCAAT | |
| | GAAGCAGGCAGAGAGGTGGTAGGGTCAGCAGAGGCTGGAGTGGATGCTGCAAGCGTCTCTGAA | |
| | GAATTTAGGGCTGACCATCCATTCCTCTTCTGTATCAAGCACATCGCAACCAACGCCGTTCTC | |
| | TTCTTTGGCAGATGTGTTTCCCCTTAA | |

TABLE 17

| | | SEQ |
|---|---|---|
| | Sequence | ID NO: |
| Signal peptide | MARSVTLVFLVLVSLTGLYA | 215 |
| of β₂ | ATGGCCAGGTCTGTGACACTGGTGTTTCTGGTGCTGGTGTCCCTGACAGGACTC | 216 |
| microglobulin | TACGCC | |
| OVA peptide | SIINFEKL | 217 |
| | TCTATCATCAATTTCGAAAAACTG | 218 |
| Linker | GGGASGGGGSGGGGS | 219 |
| | GGGGGCGGTGCCAGCGGGGGAGGCGGCTCCGGCGGGGGGGGCTCC | 220 |
| β₂ | IQKTPQIQVYSRHPPENGKPNILNCYVTQFHPPHIEIQMLKNGKKIPKVEMSDM | 221 |
| microglobulin | SFSKDWSFYILAHTEFTPTETDTYACRVKHASMAEPKTVYWDRDM | |
| (from which signal | ATCCAGAAGACCCCCCAGATCCAGGTCTACTCTCGACACCCTCCTGAGAACGGC | 222 |
| peptide is | AAGCCAAACATCCTGAACTGCTATGTGACCCAGTTCCATCCCCCACACATTGAG | |
| removed) | ATCCAGATGCTGAAGAACGGCAAGAAGATCCCAAAAGTGGAGATGTCTGATATG | |
| | TCATTTTCAAAGGACTGGAGCTTCTACATTCTCGCTCACACCGAGTTTACTCCA | |
| | ACCGAAACAGATACCTACGCTTGTCGGGTGAAACATGCCTCCATGGCAGAACCA | |
| | AAGACTGTGTACTGGGATAGGGACATG | |
| Linker | GGGGSGGGGSGGGGSGGGGS | 223 |
| | GGGGGCGGCGGGTCCGGGGGGGGCGGCAGTGGAGGCGGCGGAAGCGGCGGCGGA | 224 |
| | GGCTCA | |
| MHC class | GPHSLRYFVTAVSRPGLGEPRYMEVGYVDDTEFVRFDSDAENPRYEPRARWMEQ | 225 |
| Iα chain | EGPEYWERETQKAKGNEQSFRVDLRTLLGYYNQSKGGSHTIQVISGCEVGSDGR | |
| (from which signal | LLRGYQQYAYDGCDYIALNEDLKTWTAADMAALITKHKWEQAGEAERLRAYLEG | |
| peptide is | TCVEWLRRYLKNGNATLLRTDSPKAHVTHHSRPEDKVTLRCWALGFYPADITLT | |
| removed) | WQLNGEELIQDMELVETRPAGDGTFQKWASVVVPLGKEQYYTCHVYHQGLPEPL | |
| | TLRWEPPPSTVSNMATVAVLVVLGAAIVTGAVVAFVMKMRRRNTGGKGGDYALA | |
| | PGSQTSDLSLPDCKVMVHDPHSLA | |
| | GGGCCCCACAGCCTGCGGTACTTCGTGACCGCAGTGAGCCGCCCCGGACTGGGA | 226 |
| | GAGCCTAGGTACATGGAGGTGGGATACGTGGACGACACCGAGTTCGTCAGGTTC | |
| | GACTCTGACGCCGAAAACCCTAGGTACGAGCCCAGAGCCCGGTGGATGGAACAG | |
| | GAGGGACCAGAGTACTGGGAACGCGAGACCCAGAAGGCAAAGGGAAACGAGCAG | |
| | TCCTTCCGTGTGGACCTCCGAACGCTGCTGGGGTACTACAACCAGTCAAAGGGC | |
| | GGGTCTCATACAATCCAGGTGATCAGCGGATGTGAGGTAGGGAGCGACGGCAGG | |
| | CTGCTGCGGGGCTACCAGCAGTACGCCTATGACGGCTGCGACTACATCGCTCTG | |
| | AACGAAGATCTCAAGACCTGGACAGCCGCAGACATGGCCGCCCTCATCACTAAG | |
| | CATAAGTGGGAACAGGCTGGGGAGGCCGAGAGACTGAGAGCCTATCTGGAGGGC | |
| | ACTTGCGTGGAATGGCTGAGACGATACCTTAAGAACGGTAATGCCACACTGCTG | |
| | AGAACAGACTCCCCAAAGGCCCACGTGACCCACCACTCTAGACCAGAGGACAAG | |
| | GTGACACTCAGATGCTGGGCCCTGGGCTTCTACCCTGCTGACATTACACTCACC | |
| | TGGCAGCTGAACGGCGAGGAGCTCATCCAGGACATGGAGCTGGTGGAGACAAGG | |
| | CCCGCAGGCGACGGTACCTTCCAGAAGTGGGCAAGCGTGGTGGTCCCTCTCGGG | |
| | AAGGAGCAGTACTACACATGCCACGTGTATCATCAGGGGCTGCCTGAGCCCCTG | |
| | ACACTGAGGTGGGAGCCCCCCCCCAGCACAGTTAGCAACATGGCTACCGTGGCT | |
| | GTGCTGGTGGTGCTGGGCGCCGCCATCGTGACAGGGGCCGTGGTCGCTTTCGTG | |
| | ATGAAGATGCGGCGAAGAAATACAGGCGGCAAGGGCGGCGACTATGCTCTGGCT | |
| | CCTGGATCTCAGACCAGCGACCTCAGTCTGCCCGACTGTAAGGTGATGGTGCAC | |
| | GACCCACACTCCCTGGCA | |
| T2A | GSGEGRGSLLTCGDVEENPGP | 227 |
| | GGCAGTGGCGAAGGAAGGGGCTCCCTGCTGACCTGCGGGGACGTGGAGGAAAAC | 228 |
| | CCCGGACCT | |
| TfR (transferrin | MMDQARSAFSNLFGGEPLSYTRFSLARQVDGDNSHVEMKLAVDEEENADNNTKA | 229 |
| receptor) | NVTKPKRCSGSICYGTIAVIVFFLIGFMIGYLGYCKGVEPKTECERLAGTESPV | |
| | REEPGEDF | |
| | ATGATGGACCAGGCCAGAAGCGCGTTTAGCAATCTGTTTGGTGGCGAACCTCTG | 230 |
| | TCTTACACTAGATTCAGTCTGGCCAGGCAGGTGGACGGAGACAACTCCCACGTG | |
| | GAGATGAAGCTGGCTGTGGACGAGGAGGAGAACGCCGACAACAACACAAAGGCC | |
| | AACGTGACCAAGCCTAAGAGGTGCAGCGGTTCTATCTGTTACGGCACCATCGCT | |
| | GTGATCGTGTTTTTCCTGATAGGCTTTATGATCGGCTACCTGGGCTACTGCAAG | |
| | GGCGTGGAGCCTAAGACCGAATGCGAGCGGCTGGCCGGGACAGAGTCCCCTGTG | |
| | AGGGAGGAGCCTGGCGAGGACTTC | |
| Linker | GGGGS | 231 |
| | GGGGGCGGCGGCAGT | 232 |
| IL-15Rα sushi | TTCPPPVSIEHADIRVKNYSVNSRERYVCNSGFKRKAGTSTLIECVINKNTNVA | 233 |
| | HWTTPSLKCIRDPSLAHYSPVPT | |
| | ACAACCTGCCCCCCTCCTGTGTCCATTGAGCATGCCGACATCAGGGTGAAAAAT | 234 |
| | TATTCCGTGAACTCTAGAGAGAGATACGTGTGTAACAGCGGCTTCAAAAGAAAG | |
| | GCCGGCACATCTACCCTGATCGAATGTGTGATCAACAAGAACACAAACGTGGCA | |
| | CATTGGACTACCCCCAGCCTGAAATGCATCAGGGACCCCTCTCTGGCCCACTAC | |
| | AGTCCCGTGCCTACA | |
| Linker | SGGSGGGGSGGGSGGGGSLQ | 235 |
| | AGCGGCGGGTCCGGCGGCGGCGGCAGCGGGGGGGGTAGCGGCGGCGGCGGCTCC | 236 |
| | CTGCAG | |
| IL-15 | NWIDVRYDLEKIESLIQSIHIDTTLYTDSDFHPSCKVTAMNCFLLELQVILHEY | 237 |
| | SNMTLNETVRNVLYLANSTLSSNKNVAESGCKECEELEEKTFTEFLQSFIRIVQ | |
| | MFINTS | |
| | AATTGGATCGACGTGCGCTACGATCTGGAGAAGATCGAGTCCTTGATTCAGTCC | 238 |
| | ATTCACATAGACACCACCCTGTATACAGACAGCGACTTTCACCCATCTTGTAAG | |
| | GTGACCGCCATGAACTGTTTTCTGCTGGAGCTGCAGGTGATCCTGCATGAATAC | |
| | AGCAACATGACCCTGAACGAGACCGTGCGCAACGTGCTGTATCTGGCCAACTCC | |
| | ACCCTGAGCTCTAATAAGAACGTGGCCGAGAGCGGGTGTAAGGAGTGTGAGGAG | |
| | CTGGAGGAGAAGACCTTTACAGAGTTTCTGCAGAGCTTCATCCGGATTGTGCAG | |
| | ATGTTTATTAACACCAGC | |
| FLAG | DYKDDDDK | 239 |
| | GACTACAAGGACGACGATGACAAG | 240 |
| P2A | GSGATNFSLLKQAGDVEENPGP | 241 |
| | GGATCAGGCGCCACCAATTTTTCTCTCCTCAAGCAAGCCGGCGACGTGGAGGAG | 242 |
| | AACCCAGGCCCT | |
| CD80 | MACNCQLMQDTPLLKFPCPRLILLFVLLIRLSQVSSDVDEQLSKSVKDKVLLPC | 243 |
| | RYNSPHEDESEDRIYWQKHDKVVLSVIAGKLKVWPEYKNRTLYDNTTYSLIILG | |
| | LVLSDRGTYSCVVQKKERGTYEVKHLALVKLSIKADFSTPNITESGNPSADTKR | |
| | ITCFASGGFPKPRFSWLENGRELPGINTTISQDPESELYTISSQLDFNTTRNHT | |
| | IKCLIKYGDAHVSEDFTWEKPPEDPPDSKNTLVLFGAGFGAVITVVVIVVIIKC | |
| | FCKHRSCFRRNEASRETNNSLTFGPEEALAEQTVFL | |
| | ATGGCCTGCAATTGTCAACTGATGCAGGATACCCCCCTGCTGAAGTTTCCCTGT | 244 |
| | CCACGCCTGATCCTGCTGTTCGTGCTCCTGATCAGGCTGAGTCAGGTGTCTTCT | |
| | GACGTGGATGAGCAGCTGAGTAAGAGCGTCAAGGATAAGGTGCTGCTCCCTTGC | |
| | AGATATAATAGCCCCCATGAGGATGAGAGCGAGGACAGGATCTACTGGCAGAAG | |
| | CACGATAAGGTGGTGCTGTCTGTGATTGCTGGCAAACTGAAGGTGTGGCCTGAG | |
| | TATAAGAACAGAACCCTGTACGATAACACTACCTACAGCCTGATCATCCTGGGC | |
| | CTGGTGCTGAGCGACAGGGGCACATACTCTTGTGTGGTGCAGAAGAAAGAGAGG | |
| | GGAACCTACGAGGTGAAACATCTGGCTCTGGTAAAGCTGTCCATCAAGGCCGAC | |
| | TTCTCCACACCAAATATCACCGAGAGCGGAAACCCCTCCGCTGACACAAAGCGG | |
| | ATCACCTGTTTCGCTTCCGGTGGCTTCCCCAAACCCAGGTTCAGCTGGCTGGAG | |
| | AACGGAAGAGAGCTGCCAGGCATCAACACCACCATCAGCCAGGACCCCGAGTCT | |
| | GAGCTGTACACCATCAGCTCCCAGCTGGACTTCAACACAACCAGGAACCACACT | |
| | ATCAAGTGCCTGATCAAATATGGCGACGCCCACGTCAGCGAAGACTTCACCTGG | |
| | GAGAAGCCCCCTGAGGACCCACCCGATTCCAAGAATACACTGGTGCTTTTCGGC | |
| | GCCGGGTTTGGTGCCGTGATTACCGTGGTGGTGATTGTGGTGATTATTAAGTGC | |
| | TTCTGTAAGCACAGGTCCTGCTTCAGAAGGAACGAGGCTAGCCGCGAAACAAAC | |
| | AACAGTCTGACGTTCGGCCCCGAAGAGGCCCTGGCCGAGCAGACAGTGTTCCTC | |

TABLE 18

| | | SEQ |
|---|---|---|
| | Sequence | ID NO: |
| OVApscMHCI-T2A- | MARSVTLVFLVLVSLTGLYASIINFEKLGGGASGGGGSGGGGSIQKTPQIQVYS | 245 |
| TfR-IL-15Ra | RHPPENGKPNILNCYVTQFHPPHIEIQMLKNGKKIPKVEMSDMSFSKDWSFYIL | |
| sushi-linker-IL15- | AHTEFTPTETDTYACRVKHASMAEPKTVYWDRDMGGGGSGGGGSGGGGSGGGGS | |
| Flag-P2A-CD80 | GPHSLRYFVTAVSRPGLGEPRYMEVGYVDDTEFVRFDSDAENPRYEPRARWMEQ | |
| | EGPEYWERETQKAKGNEQSFRVDLRTLLGYYNQSKGGSHTIQVISGCEVGSDGR | |
| | LLRGYQQYAYDGCDYIALNEDLKTWTAADMAALITKHKWEQAGEAERLRAYLEG | |
| | TCVEWLRRYLKNGNATLLRTDSPKAHVTHHSRPEDKVTLRCWALGFYPADITLT | |
| | WQLNGEELIQDMELVETRPAGDGTFQKWASVVVPLGKEQYYTCHVYHQGLPEPL | |
| | TLRWEPPPSTVSNMATVAVLVVLGAAIVTGAVVAFVMKMRRRNTGGKGGDYALA | |
| | PGSQTSDLSLPDCKVMVHDPHSLAGSGEGRGSLLTCGDVEENPGPMMDQARSAF | |
| | SNLFGGEPLSYTRFSLARQVDGDNSHVEMKLAVDEEENADNNTKANVTKPKRCS | |
| | GSICYGTIAVIVFFLIGFMIGYLGYCKGVEPKTECERLAGTESPVREEPGEDFG | |
| | GGGSTTCPPPVSTEHADTRVKNYSVNSRERYVCNSGFKRKAGTSTLTECVINKN | |
| | TNVAHWTTPSLKCIRDPSLAHYSPVPTSGGSGGGGSGGGSGGGGSLQNWIDVRY | |
| | DLEKIESLIQSIHIDTTLYTDSDFHPSCKVTAMNCFLLELQVILHEYSNMTLNE | |
| | TVRNVLYLANSTLSSNKNVAESGCKECEELEEKTFTEFLQSFIRIVQMFINTSD | |
| | YKDDDDKGSGATNFSLLKQAGDVEENPGPMACNCQLMQDTPLLKFPCPRLILLF | |
| | VLLIRLSQVSSDVDEQLSKSVKDKVLLPCRYNSPHEDESEDRIYWQKHDKVVLS | |
| | VIAGKLKVWPEYKNRTLYDNTTYSLIILGLVLSDRGTYSCVVQKKERGTYEVKH | |
| | LALVKLSIKADFSTPNITESGNPSADTKRITCFASGGFPKPRFSWLENGRELPG | |
| | INTTISQDPESELYTISSQLDFNTTRNHTIKCLIKYGDAHVSEDFTWEKPPEDP | |
| | PDSKNTLVLFGAGFGAVITVVVIVVIIKCFCKHRSCFRRNEASRETNNSLTFGP | |
| | EEALAEQTVFL | |
| | ATGGCCAGGTCTGTGACACTGGTGTTTCTGGTGCTGGTGTCCCTGACAGGACTC | 246 |
| | TACGCCTCTATCATCAATTTCGAAAAACTGGGGGGCGGTGCCAGCGGGGGAGGC | |
| | GGCTCCGGCGGGGGCGGCTCCATCCAGAAGACCCCCCAGATCCAGGTCTACTCT | |
| | CGACACCCTCCTGAGAACGGCAAGCCAAACATCCTGAACTGCTATGTGACCCAG | |
| | TTCCATCCCCCACACATTGAGATCCAGATGCTGAAGAACGGCAAGAAGATCCCA | |
| | AAAGTGGAGATGTCTGATATGTCATTTTCAAAGGACTGGAGCTTCTACATTCTC | |
| | GCTCACACCGAGTTTACTCCAACCGAAACAGATACCTACGCTTGTCGGGTGAAA | |
| | CATGCCTCCATGGCAGAACCAAAGACTGTGTACTGGGATAGGGACATGGGGGGC | |
| | GGCGGGTCCGGGGGGGGCGGCAGTGGAGGCGGCGGAAGCGGCGGCGGAGGCTCA | |
| | GGGCCCCACAGCCTGCGGTACTTCGTGACCGCAGTGAGCCGCCCCGGACTGGGA | |
| | GAGCCTAGGTACATGGAGGTGGGATACGTGGACGACACCGAGTTCGTCAGGTTC | |
| | GACTCTGACGCCGAAAACCCTAGGTACGAGCCCAGAGCCCGGTGGATGGAACAG | |
| | GAGGGACCAGAGTACTGGGAACGCGAGACCCAGAAGGCAAAGGGAAACGAGCAG | |
| | TCCTTCCGTGTGGACCTCCGAACGCTGCTGGGGTACTACAACCAGTCAAAGGGC | |
| | GGGTCTCATACAATCCAGGTGATCAGCGGATGTGAGGTAGGGAGCGACGGCAGG | |
| | CTGCTGCGGGGCTACCAGCAGTACGCCTATGACGGCTGCGACTACATCGCTCTG | |
| | AACGAAGATCTCAAGACCTGGACAGCCGCAGACATGGCCGCCCTCATCACTAAG | |
| | CATAAGTGGGAACAGGCTGGGGAGGCCGAGAGACTGAGAGCCTATCTGGAGGGC | |
| | ACTTGCGTGGAATGGCTGAGACGATACCTTAAGAACGGTAATGCCACACTGCTG | |
| | AGAACAGACTCCCCAAAGGCCCACGTGACCCACCACTCTAGACCAGAGGACAAG | |
| | GTGACACTCAGATGCTGGGCCCTGGGCTTCTACCCTGCTGACATTACACTCACC | |
| | TGGCAGCTGAACGGCGAGGAGCTCATCCAGGACATGGAGCTGGTGGAGACAAGG | |
| | CCCGCAGGCGACGGTACCTTCCAGAAGTGGGCAAGCGTGGTGGTCCCTCTCGGG | |
| | AAGGAGCAGTACTACACATGCCACGTGTATCATCAGGGGCTGCCTGAGCCCCTG | |
| | ACACTGAGGTGGGAGCCCCCCCCCAGCACAGTTAGCAACATGGCTACCGTGGCT | |
| | GTGCTGGTGGTGCTGGGCGCCGCCATCGTGACAGGGGCCGTGGTCGCTTTCGTG | |
| | ATGAAGATGCGGCGAAGAAATACAGGCGGCAAGGGCGGCGACTATGCTCTGGCT | |
| | CCTGGATCTCAGACCAGCGACCTCAGTCTGCCCGACTGTAAGGTGATGGTGCAC | |
| | GACCCACACTCCCTGGCAGGCAGTGGCGAAGGAAGGGGCTCCCTGCTGACCTGC | |
| | GGGGACGTGGAGGAAAACCCCGGACCTATGATGGACCAGGCCAGAAGCGCGTTT | |
| | AGCAATCTGTTTGGTGGCGAACCTCTGTCTTACACTAGATTCAGTCTGGCCAGG | |
| | CAGGTGGACGGAGACAACTCCCACGTGGAGATGAAGCTGGCTGTGGACGAGGAG | |
| | GAGAACGCCGACAACAACACAAAGGCCAACGTGACCAAGCCTAAGAGGTGCAGC | |
| | GGTTCTATCTGTTACGGCACCATCGCTGTGATCGTGTTTTTCCTGATAGGCTTT | |
| | ATGATCGGCTACCTGGGCTACTGCAAGGGCGTGGAGCCTAAGACCGAATGCGAG | |
| | CGGCTGGCCGGGACAGAGTCCCCTGTGAGGGAGGAGCCTGGCGAGGACTTCGGG | |
| | GGCGGCGGCAGTACAACCTGCCCCCCTCCTGTGTCCATTGAGCATGCCGACATC | |
| | AGGGTGAAAAATTATTCCGTGAACTCTAGAGAGAGATACGTGTGTAACAGCGGC | |
| | TTCAAAAGAAAGGCCGGCACATCTACCCTGATCGAATGTGTGATCAACAAGAAC | |
| | ACAAACGTGGCACATTGGACTACCCCCAGCCTGAAATGCATCAGGGACCCCTCT | |
| | CTGGCCCACTACAGTCCCGTGCCTACAAGCGGCGGGTCCGGCGGCGGCGGCAGC | |
| | GGGGGGGGTAGCGGCGGCGGCGGCTCCCTGCAGAATTGGATCGACGTGCGCTAC | |
| | GATCTGGAGAAGATCGAGTCCTTGATTCAGTCCATTCACATAGACACCACCCTG | |
| | TATACAGACAGCGACTTTCACCCATCTTGTAAGGTGACCGCCATGAACTGTTTT | |
| | CTGCTGGAGCTGCAGGTGATCCTGCATGAATACAGCAACATGACCCTGAACGAG | |
| | ACCGTGCGCAACGTGCTGTATCTGGCCAACTCCACCCTGAGCTCTAATAAGAAC | |
| | GTGGCCGAGAGCGGGTGTAAGGAGTGTGAGGAGCTGGAGGAGAAGACCTTTACA | |
| | GAGTTTCTGCAGAGCTTCATCCGGATTGTGCAGATGTTTATTAACACCAGCGAC | |
| | TACAAGGACGACGATGACAAGGGATCAGGCGCCACCAATTTTTCTCTCCTCAAG | |
| | CAAGCCGGCGACGTGGAGGAGAACCCAGGCCCTATGGCCTGCAATTGTCAACTG | |
| | ATGCAGGATACCCCCCTGCTGAAGTTTCCCTGTCCACGCCTGATCCTGCTGTTC | |
| | GTGCTCCTGATCAGGCTGAGTCAGGTGTCTTCTGACGTGGATGAGCAGCTGAGT | |
| | AAGAGCGTCAAGGATAAGGTGCTGCTCCCTTGCAGATATAATAGCCCCCATGAG | |
| | GATGAGAGCGAGGACAGGATCTACTGGCAGAAGCACGATAAGGTGGTGCTGTCT | |
| | GTGATTGCTGGCAAACTGAAGGTGTGGCCTGAGTATAAGAACAGAACCCTGTAC | |
| | GATAACACTACCTACAGCCTGATCATCCTGGGCCTGGTGCTGAGCGACAGGGGC | |
| | ACATACTCTTGTGTGGTGCAGAAGAAAGAGAGGGGAACCTACGAGGTGAAACAT | |
| | CTGGCTCTGGTAAAGCTGTCCATCAAGGCCGACTTCTCCACACCAAATATCACC | |
| | GAGAGCGGAAACCCCTCCGCTGACACAAAGCGGATCACCTGTTTCGCTTCCGGT | |
| | GGCTTCCCCAAACCCAGGTTCAGCTGGCTGGAGAACGGAAGAGAGCTGCCAGGC | |
| | ATCAACACCACCATCAGCCAGGACCCCGAGTCTGAGCTGTACACCATCAGCTCC | |
| | CAGCTGGACTTCAACACAACCAGGAACCACACTATCAAGTGCCTGATCAAATAT | |
| | GGCGACGCCCACGTCAGCGAAGACTTCACCTGGGAGAAGCCCCCTGAGGACCCA | |
| | CCCGATTCCAAGAATACACTGGTGCTTTTCGGCGCCGGGTTTGGTGCCGTGATT | |
| | ACCGTGGTGGTGATTGTGGTGATTATTAAGTGCTTCTGTAAGCACAGGTCCTGC | |
| | TTCAGAAGGAACGAGGCTAGCCGCGAAACAAACAACAGTCTGACGTTCGGCCCC | |
| | GAAGAGGCCCTGGCCGAGCAGACAGTGTTCCTCTGATGATGA | |

TABLE 19

| | | SEQ |
|---|---|---|
| | Sequence | ID NO: |
| Signal peptide | MALQIPSLLLSAAVVVLMVLSSPGTEG | 247 |
| of MHC class | ATGGCTCTGCAGATACCCTCCTTGCTTCTGTCCGCTGCCGTAGTGGTGCTGATG | 248 |
| IIß chain | GTGCTTAGCAGCCCTGGGACTGAAGGT | |
| OVA peptide | ISQAVHAAHAEINEAGR | 249 |
| | ATATCTCAAGCCGTTCACGCCGCCCATGCGGAGATAAATGAAGCTGGTAGG | 250 |
| Linker | GGGGGGGGSG | 251 |
| | GGAGGCGGAGGCTCCGGAGGCGGCGGGAGCGGA | 252 |
| MHC class | GDSERHFVYQFMGECYFTNGTQRIRYVTRYIYNREEYVRYDSDVGEHRAVTELG | 253 |
| IIß chain | RPDAEYWNSQPEILERTRAELDTVCRHNYEGPETHTSLRRLEQPNVVISLSRTE | |
| (from which signal | ALNHHNTLVCSVTDFYPAKIKVRWFRNGQEETVGVSSTQLIRNGDWTFQVLVML | |
| peptide is | EMTPRRGEVYTCHVEHPSLKSPITVEWRAQSESAWSKMLSGIGGCVLGVIFLGL | |
| removed) | GLFIRHRSQKGPRGPPPAGLLQ | |
| | GGCGACAGTGAGCGTCACTTTGTCTATCAGTTCATGGGCGAGTGCTACTTTACC | 254 |
| | AATGGCACACAACGGATAAGATATGTGACCCGGTACATTTACAATCGAGAAGAG | |
| | TATGTGCGATATGACTCTGACGTCGGCGAGCATCGGGCCGTAACCGAGCTGGGC | |
| | AGACCCGATGCAGAGTATTGGAATAGCCAGCCGGAGATCCTGGAACGCACTAGG | |
| | GCCGAGCTGGACACAGTTTGCCGGCACAATTACGAGGGTCCAGAAACACACACC | |
| | TCACTGAGGAGGCTGGAGCAGCCTAACGTCGTTATCTCTCTGAGCCGCACCGAG | |
| | GCTCTCAACCATCACAACACATTGGTTTGCTCTGTGACTGATTTCTACCCAGCC | |
| | AAGATCAAGGTTCGCTGGTTTAGGAATGGGCAAGAGGAGACTGTAGGTGTCTCA | |
| | AGTACACAGCTGATCAGAAACGGAGATTGGACATTCCAGGTACTGGTTATGCTG | |
| | GAAATGACTCCTCGCAGGGGAGAGGTGTACACATGCCACGTCGAACATCCATCC | |
| | CTCAAATCTCCCATCACTGTGGAATGGAGAGCCCAGAGTGAGTCCGCATGGAGT | |
| | AAAATGCTGAGCGGAATTGGTGGATGTGTGCTCGGGGTTATTTTCTTGGGGCTC | |
| | GGGTTGTTCATTCGGCATCGCTCACAGAAAGGACCGCGAGGGCCACCACCAGCG | |
| | GGTCTGCTGCAG | |
| P2A | GSGATNFSLLKQAGDVEENPGP | 255 |
| | GGCAGCGGCGCCACAAACTTCTCTCTGCTAAAGCAAGCAGGTGATGTTGAAGAA | 256 |
| | AACCCCGGGCCT | |
| MHC class | MPRSRALILGVLALTTMLSLOGGEDDIEADHVGTYGISVYQSPGDIGQYTFEFD | 257 |
| IIα chain | GDELFYVDLDKKETVWMLPEFGQLASFDPQGGLQNIAVVKHNLGVLTKRSNSTP | |
| | ATNEAPQATVFPKSPVLLGQPNTLICFVDNIFPPVINITWLRNSKSVADGVYET | |
| | SFFVNRDYSFHKLSYLTFIPSDDDIYDCKVEHWGLEEPVLKHWEPEIPAPMSEL | |
| | TETVVCALGLSVGLVGIVVGTIFIIQGLRSGGTSRHPGPL | |
| | ATGCCCAGATCCAGAGCACTGATCCTTGGTGTTCTGGCCCTTACAACGATGCTT | 258 |
| | AGTTTGTGTGGCGGGGAGGATGACATCGAAGCTGATCATGTGGGCACGTACGGT | |
| | ATCTCCGTGTACCAGTCTCCCGGGGATATTGGACAGTATACGTTCGAATTTGAT | |
| | GGCGACGAGCTCTTCTACGTGGACCTCGATAAGAAGGAAACTGTCTGGATGTTG | |
| | CCTGAGTTTGGGCAGCTGGCATCATTTGACCCTCAAGGAGGGCTGCAGAATATC | |
| | GCGGTCGTGAAGCACAATCTTGGCGTACTGACAAAGCGATCCAACAGTACCCCG | |
| | GCTACTAACGAGGCACCCCAAGCAACCGTGTTTCCTAAGTCCCCAGTGCTGCTC | |
| | GGCCAGCCCAACACCCTCATCTGTTTCGTAGACAACATCTTTCCTCCCGTCATT | |
| | AACATCACATGGCTGCGTAACTCCAAATCAGTGGCTGATGGGGTGTATGAAACC | |
| | TCTTTCTTCGTGAACAGGGACTACAGTTTTCACAAGCTGTCTTATCTCACGTTC | |
| | ATCCCTAGCGACGATGACATTTACGACTGCAAAGTGGAACATTGGGGCCTGGAA | |
| | GAGCCTGTCCTGAAGCACTGGGAACCGGAGATTCCCGCCCCTATGAGCGAGCTG | |
| | ACAGAAACCGTTGTCTGTGCACTGGGTCTGTCAGTCGGCCTCGTGGGAATTGTG | |
| | GTCGGTACCATATTCATCATTCAGGGACTGAGAAGCGGAGGCACCAGCCGTCAC | |
| | CCCGGACCACTC | |
| T2A | GSGEGRGSLLTCGDVEENPGP | 259 |
| | GGCTCCGGCGAGGGCAGGGGAAGTCTTCTAACATGCGGGGACGTGGAGGAAAAT | 260 |
| | CCCGGCCCA | |
| IL-12β | MCPQKLTISWFAIVLLVSPLMAMWELEKDVYVVEVDWTPDAPGETVNLTCDTPE | 261 |
| | EDDITWTSDQRHGVIGSGKTLTITVKEFLDAGQYTCHKGGETLSHSHLLLHKKE | |
| | NGIWSTEILKNFKNKTFLKCEAPNYSGRFTCSWLVQRNMDLKFNIKSSSSSPDS | |
| | RAVTCGMASLSAEKVTLDQRDYEKYSVSCQEDVTCPTAEETLPIELALEARQQN | |
| | KYENYSTSFFIRDIIKPDPPKNLQMKPLKNSQVEVSWEYPDSWSTPHSYFSLKF | |
| | FVRIQRKKEKMKETEEGCNQKGAFLVEKTSTEVQCKGGNVCVQAQDRYYNSSCS | |
| | KWACVPCRVRS | |
| | ATGTGTCCACAGAAGCTGACCATCTCATGGTTTGCCATAGTGCTGCTGGTGTCC | 262 |
| | CCACTGATGGCCATGTGGGAGCTGGAGAAGGACGTGTACGTCGTGGAGGTGGAC | |
| | TGGACCCCCGATGCTCCAGGCGAGACAGTGAACCTGACCTGCGACACCCCCGAG | |
| | GAGGACGATATCACCTGGACATCCGATCAGAGACACGGAGTGATCGGCTCCGGC | |
| | AAGACCCTGACTATTACCGTGAAGGAATTTCTGGACGCTGGGCAGTACACTTGT | |
| | CACAAAGGCGGAGAGACACTGTCTCATTCTCACCTGCTGCTGCATAAAAAGGAG | |
| | AACGGGATCTGGAGCACTGAAATCCTGAAGAACTTTAAGAACAAGACCTTCCTG | |
| | AAGTGCGAGGCCCCCAACTACAGCGGGAGATTCACATGCAGCTGGCTGGTGCAG | |
| | CGCAACATGGATCTGAAGTTTAACATCAAGTCCAGCAGTTCATCTCCAGACAGC | |
| | AGGGCAGTGACATGCGGCATGGCTTCCCTGTCTGCCGAGAAGGTGACCCTGGAC | |
| | CAGCGCGATTATGAGAAGTACAGCGTGAGCTGCCAGGAGGATGTGACCTGTCCT | |
| | ACCGCCGAGGAGACACTGCCAATCGAACTGGCCCTGGAAGCTAGACAGCAGAAC | |
| | AAGTACGAGAATTACTCCACCTCCTTTTTCATCCGTGACATTATCAAGCCGGAC | |
| | CCTCCAAAGAACCTGCAGATGAAGCCACTGAAAAATTCCCAGGTGGAAGTGAGC | |
| | TGGGAGTACCCCGATTCCTGGAGCACACCCCACTCCTACTTCAGCCTGAAGTTC | |
| | TTCGTGAGAATCCAGAGGAAGAAGGAGAAGATGAAAGAGACTGAGGAGGGCTGT | |
| | AACCAGAAAGGCGCATTCCTGGTGGAGAAAACCTCTACCGAAGTGCAGTGCAAG | |
| | GGGGGCAATGTGTGTGTGCAGGCCCAGGACAGGTACTATAATTCTTCCTGCAGC | |
| | AAGTGGGCATGCGTGCCATGTAGGGTGAGAAGT | |
| Linker | GGGGSGGGGSGGGGS | 263 |
| | GGAGGCGGTGGAAGCGGAGGCGGCGGGTCTGGCGGGGGTGGCAGC | 264 |
| IL-12α | RVIPVSGPARCLSQSRNLLKTTDDMVKTAREKLKHYSCTAEDIDHEDITRDQTS | 265 |
| | TLKTCLPLELHKNESCLATRETSSTTRGSCLPPQKTSLMMTLCLGSIYEDLKMY | |
| | QTEFQAINAALQNHNHQQIILDKGMLVAIDELMQSLNHNGETLRQKPPVGEADP | |
| | YRVKMKLCILLHAFSTRVVTINRVMGYLSSA | |
| | CGGGTGATCCCTGTGAGCGGCCCAGCACGCTGTCTGAGTCAGTCTCGGAACCTG | 266 |
| | CTGAAAACCACCGACGATATGGTGAAGACCGCTAGAGAAAAGCTGAAGCATTAC | |
| | AGCTGTACTGCTGAGGATATTGACCACGAGGATATCACTCGGGATCAGACTAGC | |
| | ACCCTGAAGACATGTCTGCCCCTGGAGCTGCACAAGAACGAGTCTTGTCTGGCA | |
| | ACCAGGGAGACCAGCAGTACCACAAGGGGCAGCTGCCTGCCTCCCCAGAAGACT | |
| | AGCCTCATGATGACACTGTGCCTGGGCAGCATCTATGAGGATCTCAAGATGTAC | |
| | CAGACCGAGTTCCAGGCCATCAACGCCGCCCTCCAGAATCACAATCACCAGCAG | |
| | ATTATCCTGGACAAGGGCATGCTGGTGGCCATTGACGAGCTCATGCAGTCTCTG | |
| | AATCACAACGGCGAGACACTGAGACAGAAGCCACCCGTCGGGGAGGCTGACCCT | |
| | TATCGGGTTAAAATGAAGCTGTGTATCCTGCTGCACGCCTTTTCCACTAGAGTG | |
| | GTGACCATTAATCGCGTCATGGGGTACCTGAGCTCCGCC | |
| Linker | GGGGS | 267 |
| | GGCGGCGGCGGCAGC | 268 |
| FLAG | DYKDDDDK | 269 |
| | GACTACAAGGACGACGACGACAAG | 270 |
| Transmembrane | VISNSVMYFSSVVPVLQKVNSTTTKPVLRTPSPVHPTGTSQPQRPEDCRPRGSV | 271 |
| domain of CD8 | KGTGLDFACDIYIWAPLAGICVALLLSLIITLICYHRSRKRVCKCPRPLVRQEG | |
| | KPRPSEKIV | |
| | GTGATCTCTAACAGCGTGATGTACTTCAGCAGCGTGGTGCCCGTGCTGCAGAAA | 272 |
| | GTGAATAGTACAACCACAAAACCCGTGCTGAGAACCCCCAGCCCAGTGCACCCA | |
| | ACAGGCACCTCTCAGCCTCAAAGACCTGAAGACTGCCGCCCTAGGGGCTCCGTG | |
| | AAGGGCACCGGACTGGACTTCGCTTGCGATATCTACATCTGGGCCCCCCTGGCA | |
| | GGCATCTGCGTGGCCCTGCTGCTGAGCCTGATCATTACCCTGATCTGCTATCAC | |
| | AGGTCCAGGAAGCGCGTGTGCAAGTGCCCCAGGCCACTGGTGAGACAGGAGGGC | |
| | AAACCCAGGCCAAGCGAAAAGATTGTC | |
| P2A | GSGATNFSLLKQAGDVEENPGP | 273 |
| | GGATCAGGCGCCACCAATTTTTCTCTCCTCAAGCAAGCCGGCGACGTGGAGGAG | 274 |
| | AACCCAGGCCCT | |
| CD80 | MACNCQLMQDTPLLKFPCPRLILLFVLLIRLSQVSSDVDEQLSKSVKDKVLLPC | 275 |
| | RYNSPHEDESEDRIYWQKHDKVVLSVIAGKLKVWPEYKNRTLYDNTTYSLIILG | |
| | LVLSDRGTYSCVVQKKERGTYEVKHLALVKLSIKADFSTPNITESGNPSADTKR | |
| | ITCFASGGFPKPRFSWLENGRELPGINTTISQDPESELYTISSQLDFNTTRNHT | |
| | IKCLIKYGDAHVSEDFTWEKPPEDPPDSKNTLVLFGAGFGAVITVVVIVVIIKC | |
| | FCKHRSCFRRNEASRETNNSLTFGPEEALAEQTVFL | |
| | ATGGCCTGCAATTGTCAACTGATGCAGGATACCCCCCTGCTGAAGTTTCCCTGT | 276 |
| | CCACGCCTGATCCTGCTGTTCGTGCTCCTGATCAGGCTGAGTCAGGTGTCTTCT | |
| | GACGTGGATGAGCAGCTGAGTAAGAGCGTCAAGGATAAGGTGCTGCTCCCTTGC | |
| | AGATATAATAGCCCCCATGAGGATGAGAGCGAGGACAGGATCTACTGGCAGAAG | |
| | CACGATAAGGTGGTGCTGTCTGTGATTGCTGGCAAACTGAAGGTGTGGCCTGAG | |
| | TATAAGAACAGAACCCTGTACGATAACACTACCTACAGCCTGATCATCCTGGGC | |
| | CTGGTGCTGAGCGACAGGGGCACATACTCTTGTGTGGTGCAGAAGAAAGAGAGG | |
| | GGAACCTACGAGGTGAAACATCTGGCTCTGGTAAAGCTGTCCATCAAGGCCGAC | |
| | TTCTCCACACCAAATATCACCGAGAGCGGAAACCCCTCCGCTGACACAAAGCGG | |
| | ATCACCTGTTTCGCTTCCGGTGGCTTCCCCAAACCCAGGTTCAGCTGGCTGGAG | |
| | AACGGAAGAGAGCTGCCAGGCATCAACACCACCATCAGCCAGGACCCCGAGTCT | |
| | GAGCTGTACACCATCAGCTCCCAGCTGGACTTCAACACAACCAGGAACCACACT | |
| | ATCAAGTGCCTGATCAAATATGGCGACGCCCACGTCAGCGAAGACTTCACCTGG | |
| | GAGAAGCCCCCTGAGGACCCACCCGATTCCAAGAATACACTGGTGCTTTTCGGC | |
| | GCCGGGTTTGGTGCCGTGATTACCGTGGTGGTGATTGTGGTGATTATTAAGTGC | |
| | TTCTGTAAGCACAGGTCCTGCTTCAGAAGGAACGAGGCTAGCCGCGAAACAAAC | |
| | AACAGTCTGACGTTCGGCCCCGAAGAGGCCCTGGCCGAGCAGACAGTGTTCCTC | |
| | TGATGATGA | |

TABLE 20-1

| | | SEQ |
|---|---|---|
| | Sequence | ID NO: |
| OVAp-MHCIIß-P2A- | MALQIPSLLLSAAVVVLMVLSSPGTEGISQAVHAAHAEINEAGRGGGGSGGGGS | 277 |
| MHCIIα-T2A-IL-12 | GGDSERHFVYQFMGECYFTNGTQRIRYVTRYIYNREEYVRYDSDVGEHRAVTEL | |
| single chain- | GRPDAEYWNSQPEILERTRAELDTVCRHNYEGPETHTSLRRLEQPNVVISLSRT | |
| CD8-P2A-CD80 | EALNHHNTLVCSVTDFYPAKIKVRWFRNGQEETVGVSSTQLIRNGDWTFQVLVM | |
| | LEMTPRRGEVYTCHVEHPSLKSPITVEWRAQSESAWSKMLSGIGGCVLGVIFLG | |
| | LGLFIRHRSQKGPRGPPPAGLLQGSGATNFSLLKQAGDVEENPGPMPRSRALIL | |
| | GVLALTTMLSLCGGEDDIEADHVGTYGISVYQSPGDIGQYTFEFDGDELFYVDL | |
| | DKKETVWMLPEFGQLASFDPQGGLQNIAVVKHNLGVLTKRSNSTPATNEAPQAT | |
| | VFPKSPVLLGQPNTLICFVDNIFPPVINITWLRNSKSVADGVYETSFFVNRDYS | |
| | FHKLSYLTFIPSDDDIYDCKVEHWGLEEPVLKHWEPEIPAPMSELTETVVCALG | |
| | LSVGLVGIVVGTIFIIQGLRSGGTSRHPGPLGSGEGRGSLLTCGDVEENPGPMC | |
| | PQKLTISWFAIVLLVSPLMAMWELEKDVYVVEVDWTPDAPGETVNLTCDTPEED | |
| | DITWTSDQRHGVIGSGKTLTITVKEFLDAGQYTCHKGGETLSHSHLLLHKKENG | |
| | IWSTEILKNFKNKTFLKCEAPNYSGRFTCSWLVQRNMDLKFNIKSSSSSPDSRA | |
| | VTCGMASLSAEKVTLDQRDYEKYSVSCQEDVTCPTAEETLPIELALEARQQNKY | |
| | ENYSTSFFIRDIIKPDPPKNLQMKPLKNSQVEVSWEYPDSWSTPHSYFSLKFFV | |
| | RIQRKKEKMKETEEGCNQKGAFLVEKTSTEVQCKGGNVCVQAQDRYYNSSCSKW | |
| | AC | |
| | VPCRVRSGGGGSGGGGSGGGGSRVIPVSGPARCLSQSRNLLKTTDDMVKTAREK | |
| | LKHYSCTAEDIDHEDITRDQTSTLKTCLPLELHKNESCLATRETSSTTRGSCLP | |
| | PQKTSLMMTLCLGSIYEDLKMYQTEFQAINAALQNHNHQQIILDKGMLVAIDEL | |
| | MQSLNHNGETLRQKPPVGEADPYRVKMKLCILLHAFSTRVVTINRVMGYLSSAG | |
| | GGGSDYKDDDDKVISNSVMYFSSVVPVLQKVNSTTTKPVLRTPSPVHPTGTSQP | |
| | QRPEDCRPRGSVKGTGLDFACDIYIWAPLAGICVALLLSLIITLICYHRSRKRV | |
| | CKCPRPLVRQEGKPRPSEKIVGSGATNFSLLKQAGDVEENPGPMACNCQLMQDT | |
| | PLLKFPCPRLILLFVLLIRLSQVSSDVDEQLSKSVKDKVLLPCRYNSPHEDESE | |
| | DRIYWQKHDKVVLSVIAGKLKVWPEYKNRTLYDNTTYSLIILGLVLSDRGTYSC | |
| | VVQKKERGTYEVKHLALVKLSIKADFSTPNITESGNPSADTKRITCFASGGFPK | |
| | PRFSWLENGRELPGINTTISQDPESELYTISSQLDFNTTRNHTIKCLIKYGDAH | |
| | VSEDFTWEKPPEDPPDSKNTLVLFGAGFGAVITVVVIVVIIKCFCKHRSCFRRN | |
| | EASRETNNSLTFGPEEALAEQTVFL | |
| | ATGGCTCTGCAGATACCCTCCTTGCTTCTGTCCGCTGCCGTAGTGGTGCTGATG | 278 |
| | GTGCTTAGCAGCCCTGGGACTGAAGGTATATCTCAAGCCGTTCACGCCGCCCAT | |
| | GCGGAGATAAATGAAGCTGGTAGGGGAGGCGGAGGCTCCGGAGGCGGCGGGAGC | |
| | GGAGGCGACAGTGAGCGTCACTTTGTCTATCAGTTCATGGGCGAGTGCTACTTT | |
| | ACCAATGGCACACAACGGATAAGATATGTGACCCGGTACATTTACAATCGAGAA | |
| | GAGTATGTGCGATATGACTCTGACGTCGGCGAGCATCGGGCCGTAACCGAGCTG | |
| | GGCAGACCCGATGCAGAGTATTGGAATAGCCAGCCGGAGATCCTGGAACGCACT | |
| | AGGGCCGAGCTGGACACAGTTTGCCGGCACAATTACGAGGGTCCAGAAACACAC | |
| | ACCTCACTGAGGAGGCTGGAGCAGCCTAACGTCGTTATCTCTCTGAGCCGCACC | |
| | GAGGCTCTCAACCATCACAACACATTGGTTTGCTCTGTGACTGATTTCTACCCA | |
| | GCCAAGATCAAGGTTCGCTGGTTTAGGAATGGGCAAGAGGAGACTGTAGGTGTC | |
| | TCAAGTACACAGCTGATCAGAAACGGAGATTGGACATTCCAGGTACTGGTTATG | |
| | CTGGAAATGACTCCTCGCAGGGGAGAGGTGTACACATGCCACGTCGAACATCCA | |
| | TCCCTCAAATCTCCCATCACTGTGGAATGGAGAGCCCAGAGTGAGTCCGCATGG | |
| | AGTAAAATGCTGAGCGGAATTGGTGGATGTGTGCTCGGGGTTATTTTCTTGGGG | |
| | CTCGGGTTGTTCATTCGGCATCGCTCACAGAAAGGACCGCGAGGGCCACCACCA | |
| | GCGGGTCTGCTGCAGggcagcggcgccacaaacttctctctgctaaagcaagca | |
| | ggtgatgttgaagaaaaccccgggcctATGCCCAGATCCAGAGCACTGATCCTT | |
| | GGTGTTCTGGCCCTTACAACGATGCTTAGTTTGTGTGGCGGGGAGGATGACATC | |
| | GAAGCTGATCATGTGGGCACGTACGGTATCTCCGTGTACCAGTCTCCCGGGGAT | |
| | ATTGGACAGTATACGTTCGAATTTGATGGCGACGAGCTCTTCTACGTGGACCTC | |
| | GATAAGAAGGAAACTGTCTGGATGTTGCCTGAGTTTGGGCAGCTGGCATCATTT | |
| | GACCCTCAAGGAGGGCTGCAGAATATCGCGGTCGTGAAGCACAATCTTGGCGTA | |
| | CTGACAAAGCGATCCAACAGTACCCCGGCTACTAACGAGGCACCCCAAGCAACC | |
| | GTGTTTCCTAAGTCCCCAGTGCTGCTCGGCCAGCCCAACACCCTCATCTGTTTC | |
| | GTAGACAACATCTTTCCTCCCGTCATTAACATCACATGGCTGCGTAACTCCAAA | |
| | TCAGTGGCTGATGGGGTGTATGAAACCTCTTTCTTCGTGAACAGGGACTACAGT | |
| | TTTCACAAGCTGTCTTATCTCACGTTCATCCCTAGCGACGATGACATTTACGAC | |
| | TGCAAAGTGGAACATTGGGGCCTGGAAGAGCCTGTCCTGAAGCACTGGGAACCG | |
| | GAGATTCCCGCCCCTATGAGCGAGCTGACAGAAACCGTTGTCTGTGCACTGGGT | |
| | CTGTCAGTCGGCCTCGTGGGAATTGTGGTCGGTACCATATTCATCATTCAGGGA | |
| | CTGAGAAGCGGAGGCACCAGCCGTCACCCCGGACCACTCggctccggcgagggc | |
| | aggggaagtcttctaacatgcggggacgtggaggaaaatcccggcccaATGTGT | |
| | CCACAGAAGCTGACCATCTCATGGTTTGCCATAGTGCTGCTGGTGTCCCCACTG | |
| | ATGGCCATGTGGGAGCTGGAGAAGGACGTGTACGTCGTGGAGGTGGACTGGACC | |
| | CCCGATGCTCCAGGCGAGACAGTGAACCTGACCTGCGACACCCCCGAGGAGGAC | |
| | GATATCACCTGGACATCCGATCAGAGACACGGAGTGATCGGCTCCGGCAAGACC | |
| | CTGACTATTACCGTGAAGGAATTTCTGGACGCTGGGCAGTACACTTGTCACAAA | |
| | GGCGGAGAGACACTGTCTCATTCTCACCTGCTGCTGCATAAAAAGGAGAACGGG | |
| | ATCTGGAGCACTGAAATCCTGAAGAACTTTAAGAACAAGACCTTCCTGAAGTGC | |
| | GAGGCCCCCAACTACAGCGGGAGATTCACATGCAGCTGGCTGGTGCAGCGCAAC | |
| | ATGGATCTGAAGTTTAACATCAAGTCCAGCAGTTCATCTCCAGACAGCAGGGCA | |
| | GTGACATGCGGCATGGCTTCCCTGTCTGCCGAGAAGGTGACCCTGGACCAGCGC | |
| | GATTATGAGAAGTACAGCGTGAGCTGCCAGGAGGATGTGACCTGTCCTACCGCC | |
| | GAGGAGACACTGCCAATCGAACTGGCCCTGGAAGCTAGACAGCAGAACAAGTAC | |
| | GAGAATTACTCCACCTCCTTTTTCATCCGTGACATTATCAAGCCGGACCCTCCA | |
| | AAGAACCTGCAGATGAAGCCACTGAAAAATTCCCAGGTGGAAGTGAGCTGGGAG | |
| | TACCCCGATTCCTGGAGCACACCCCACTCCTACTTCAGCCTGAAGTTCTTCGTG | |
| | AGAATCCAGAGGAAGAAGGAGAAGATGAAAGAGACTGAGGAGGGCTGTAACCAG | |
| | AAAGGCGCATTCCTGGTGGAGAAAACCTCTACCGAAGTGCAGTGCAAGGGGGGC | |
| | AATGTGTGTGTGCAGGCCCAGGACAGGTACTATAATTCTTCCTGCAGCAAGTGG | |
| | GCATGCGTGCCATGTAGGGTGAGAAGTGGAGGCGGTGGAAGCGGAGGCGGCGGG | |
| | TCTGGCGGGGGTGGCAGCCGGGTGATCCCTGTGAGCGGCCCAGCACGCTGTCTG | |
| | AGTCAGTCTCGGAACCTGCTGAAAACCACCGACGATATGGTGAAGACCGCTAGA | |
| | GAAAAGCTGAAGCATTACAGCTGTACTGCTGAGGATATTGACCACGAGGATATC | |
| | ACTCGGGATCAGACTAGCACCCTGAAGACATGTCTGCCCCTGGAGCTGCACAAG | |
| | AACGAGTCTTGTCTGGCAACCAGGGAGACCAGCAGTACCACAAGGGGCAGCTGC | |
| | CTGCCTCCCCAGAAGACTAGCCTCATGATGACACTGTGCCTGGGCAGCATCTAT | |
| | GAGGATCTCAAGATGTACCAGACCGAGTTCCAGGCCATCAACGCCGCCCTCCAG | |
| | AATCACAATCACCAGCAGATTATCCTGGACAAGGGCATGCTGGTGGCCATTGAC | |
| | GAGCTCATGCAGTCTCTGAATCACAACGGCGAGACACTGAGACAGAAGCCACCC | |
| | GTCGGGGAGGCTGACCCTTATCGGGTTAAAATGAAGCTGTGTATCCTGCTGCAC | |
| | GCCTTTTCCACTAGAGTGGTGACCATTAATCGCGTCATGGGGTACCTGAGCTCC | |
| | GCCGGCGGCGGCGGCAGCGACTACAAGGACGACGACGACAAGGTGATCTCTAAC | |
| | AGCGTGATGTACTTCAGCAGCGTGGTGCCCGTGCTGCAGAAAGTGAATAGTACA | |
| | ACCACAAAACCCGTGCTGAGAACCCCCAGCCCAGTGCACCCAACAGGCACCTCT | |
| | CAGCCTCAAAGACCTGAAGACTGCCGCCCTAGGGGCTCCGTGAAGGGCACCGGA | |
| | CTGGACTTCGCTTGCGATATCTACATCTGGGCCCCCCTGGCAGGCATCTGCGTG | |
| | GCCCTGCTGCTGAGCCTGATCATTACCCTGATCTGCTATCACAGGTCCAGGAAG | |
| | CGCGTGTGCAAGTGCCCCAGGCCACTGGTGAGACAGGAGGGCAAACCCAGGCCA | |
| | AGCGAAAAGATTGTCGGATCAGGCGCCACCAATTTTTCTCTCCTCAAGCAAGCC | |
| | GGCGACGTGGAGGAGAACCCAGGCCCTATGGCCTGCAATTGTCAACTGATGCAG | |
| | GATACCCCCCTGCTGAAGTTTCCCTGTCCACGCCTGATCCTGCTGTTCGTGCTC | |
| | CTGATCAGGCTGAGTCAGGTGTCTTCTGACGTGGATGAGCAGCTGAGTAAGAGC | |
| | GTCAAGGATAAGGTGCTGCTCCCTTGCAGATATAATAGCCCCCATGAGGATGAG | |
| | AGCGAGGACAGGATCTACTGGCAGAAGCACGATAAGGTGGTGCTGTCTGTGATT | |
| | GCTGGCAAACTGAAGGTGTGGCCTGAGTATAAGAACAGAACCCTGTACGATAAC | |
| | ACTACCTACAGCCTGATCATCCTGGGCCTGGTGCTGAGCGACAGGGGCACATAC | |
| | TCTTGTGTGGTGCAGAAGAAAGAGAGGGGAACCTACGAGGTGAAACATCTGGCT | |
| | CTGGTAAAGCTGTCCATCAAGGCCGACTTCTCCACACCAAATATCACCGAGAGC | |
| | GGAAACCCCTCCGCTGACACAAAGCGGATCACCTGTTTCGCTTCCGGTGGCTTC | |
| | CCCAAACCCAGGTTCAGCTGGCTGGAGAACGGAAGAGAGCTGCCAGGCATCAAC | |
| | ACCACCATCAGCCAGGACCCCGAGTCTGAGCTGTACACCATCAGCTCCCAGCTG | |
| | GACTTCAACACAACCAGGAACCACACTATCAAGTGCCTGATCAAATATGGCGAC | |
| | GCCCACGTCAGCGAAGACTTCACCTGGGAGAAGCCCCCTGAGGACCCACCCGAT | |
| | TCCAAGAATACACTGGTGCTTTTCGGCGCCGGGTTTGGTGCCGTGATTACCGTG | |
| | GTGGTGATTGTGGTGATTATTAAGTGCTTCTGTAAGCACAGGTCCTGCTTCAGA | |
| | AGGAACGAGGCTAGCCGCGAAACAAACAACAGTCTGACGTTCGGCCCCGAAGAG | |
| | GCCCTGGCCGAGCAGACAGTGTTCCTCTGATGATGA | |

TABLE 21-1

| | | SEQ |
|---|---|---|
| | Sequence | ID NO: |
| Gtf2i peptide | STYVIPRL | 279 |
| | TCGACGTACGTCATTCCTCGCCTT | 280 |
| sc-Trimer (Gtf2i)- | MARSVTLVFLVLVSLTGLYASTYVIPRLGGGASGGGGSGGGGSIQKTPQIQVYS | 281 |
| T2A-IL-2-CD8- | RHPPENGKPNILNCYVTQFHPPHIEIQMLKNGKKIPKVEMSDMSFSKDWSFYIL | |
| P2A-CD80 | AHTEFTPTETDTYACRVKHASMAEPKTVYWDRDMGGGGSGGGGSGGGGSGGGGS | |
| | GPHSLRYFVTAVSRPGLGEPRYMEVGYVDDTEFVRFDSDAENPRYEPRARWMEQ | |
| | EGPEYWERETQKAKGNEQSFRVDLRTLLGYYNQSKGGSHTIQVISGCEVGSDGR | |
| | LLRGYQQYAYDGCDYIALNEDLKTWTAADMAALITKHKWEQAGEAERLRAYLEG | |
| | TCVEWLRRYLKNGNATLLRTDSPKAHVTHHSRPEDKVTLRCWALGFYPADITLT | |
| | WQLNGEELIQDMELVETRPAGDGTFQKWASVVVPLGKEQYYTCHVYHQGLPEPL | |
| | TLRWEPPPSTVSNMATVAVLVVLGAAIVTGAVVAFVMKMRRRNTGGKGGDYALA | |
| | PGSQTSDLSLPDCKVMVHDPHSLAGSGEGRGSLLTCGDVEENPGPMYSMQLASC | |
| | VTLTLVLLVNSAPTSSSTSSSTAEAQQQQQQQQQQQQHLEQLLMDLQELLSRME | |
| | NYRNLKLPRMLTFKFYLPKQATELKDLQCLEDELGPLRHVLDLTQSKSFQLEDA | |
| | ENFISNIRVTVVKLKGSDNTFECQFDDESATVVDFLRRWIAFCQSIISTSPQGG | |
| | GGSVISNSVMYFSSVVPVLQKVNSTTTKPVLRTPSPVHPTGTSQPQRPEDCRPR | |
| | GSVKGTGLDFACDIYIWAPLAGICVALLLSLIITLICYHRSRKRVCKCPRPLVR | |
| | QEGKPRPSEKIVGSGATNFSLLKQAGDVEENPGPMACNCQLMQDTPLLKFPCPR | |
| | LILLFVLLIRLSQVSSDVDEQLSKSVKDKVLLPCRYNSPHEDESEDRIYWQKHD | |
| | KVVLSVIAGKLKVWPEYKNRTLYDNTTYSLIILGLVLSDRGTYSCVVQKKERGT | |
| | YEVKHLALVKLSIKADFSTPNITESGNPSADTKRITCFASGGFPKPRFSWLENG | |
| | RELPGINTTISQDPESELYTISSQLDFNTTRNHTIKCLIKYGDAHVSEDFTWEK | |
| | PPEDPPDSKNTLVLFGAGFGAVITVVVIVVIIKCFCKHRSCFRRNEASRETNNS | |
| | LTFGPEEALAEQTVFL | |
| | atggctcgctcggtgaccctggtctttctggtgcttgtctcactgaccggcctg | 282 |
| | tatgctTCGACGTACGTCATTCCTCGCCTTGGCGGAGGTGCCTCTGGCGGTGGG | |
| | GGCAGCGGTGGAGGGGGCAGTatccagaaaacccctcaaattcaagtatactca | |
| | cgccacccaccggagaatgggaagccgaacatactgaactgctacgtaacacag | |
| | ttocacccgcctcacattgaaatccaaatgctgaagaacgggaaaaaaaTtcct | |
| | aaagtagagatgtcagatatgtccttcagcaaggactggtctttctatatcctg | |
| | gctcacactgaattcacccccactgagactgatacatacgcctgcagagttaag | |
| | catgccagtatggccgagcccaagaccgtctactgggatcgagacatgGGGGGG | |
| | GGAGGCTCCGGTGGAGGGGGGTCTGGAGGGGGGGGGTCTGGTGGAGGCGGAAGT | |
| | ggcccacactcgctgaggtatttcgtcaccgccgtgtcccggcccggcctcggg | |
| | gagccccggtacatggaagtcggctacgtggacgacacggagttcgtgcgcttc | |
| | Gacagcgacgcggagaatccgagatatgagccgcgggcgcggtggatggagcag | |
| | gaggggcccgagtattgggagcgggagacacagaaagccaagggcaatgagcag | |
| | agtttccgagtggacctgaggaccctgctoggctactacaaccagagcaagggc | |
| | ggctctcacactattcaggtgatctctggctgtgaagtggggtccgacgggcga | |
| | ctcctcegcgggtaccagcagtacgcctacgacggetgcgattacategccctg | |
| | aacgaagacctgaaaacgtggacggcggcggacatggeggcgctgatcaccaaa | |
| | cacaagtgggagcaggctggtgaagcagagagactcagggcctacctggagggc | |
| | acgtgcgtggagtggctccgcagatacctgaagaacgggaacgcgacgctgctg | |
| | cgcacagattccccaaaggcccatgtgaCccatcacagcagacctgaagataAa | |
| | gtcaccctgaggtgctgggccctgggcttctaccctgctgacatcaccctgacc | |
| | tggcagttgaatggggaggagctgatccaggacatggagcttgtggagaccagg | |
| | cctgcaggggatggaaccttccagaagtgggcatctgtggtggtgcctcttggg | |
| | aaggagcagtattacacatgccatgtgtaccatcaggggctgcctgagcccctc | |
| | accctgagatgggagcctcctccatccactgtctccaacatggcgaccgttgct | |
| | gttctggttgtccttggagctgcaatagtcactggagctgtggtggcttttgtg | |
| | atgaagatgagaaggagaaacacaggtggaaaaggaggggactatgctctggct | |
| | ccaggctcccagacctctgaTctgtctctcccagattgtaaagtgatggttcat | |
| | gaccctcattctctagegggctccggcgagggcaggggaagtcttctaacatgc | |
| | ggggacgtggaggaaaatcccggcccaATGTACTCAATGCAGCTGGCTAGTTGT | |
| | GTGACCCTGACCCTCGTGCTGCTCGTGAACAGCGCCCCAACCTCAAGCTCTACC | |
| | TCCAGTAGCACAGCCGAAGCTCAGCAGCAGCAGCAACAGCAGCAGCAGCAGCAG | |
| | CAGCACCTGGAGCAGCTGCTGATGGACCTGCAGGAGCTGCTGAGCCGGATGGAG | |
| | AACTACAGGAaCCTGAAGCTGCCTAGGATGCTGACATTCAAGTTCTACCTGCCA | |
| | AAGCAGGCCACCGAGCTGAAGGACCTGCAGTGTCTGGAGGACGAGCTGGGCCCC | |
| | CTGCGCCACGTGCTCGACCTGACACAGTCCAAGTCCTTCCAGCTGGAGGACGCA | |
| | GAGAACTTCATCTCCAACATCAGAGTGACCGTGGTGAAACTGAAGGGCTCTGAC | |
| | AACACCTTTGAGTGTCAGTTCGACGACGAGAGTGCCACTGTGGTGGATTTCCTG | |
| | AGGCGGTGGATCGCTTTCTGCCAGAGCATTATCTCTACCAGCCCACAGGGTGGC | |
| | GGCGGAAGCGTCATCAGCAACAGCGTGATGTATTTCTCCTCTGTGGTGCCTGTG | |
| | CTGCAGAAGGTGAACAGCACCACCACCAAGCCTGTGCTGAGGACTCCAAGCCCC | |
| | GTGCACCCCACTGGTACTAGCCAGCCTCAGCGCCCCGAGGACTGTAGACCTAGA | |
| | GGATCTGTGAAAGGAACAGGCCTGGACTTTGCATGTGACATCTATATTTGGGCA | |
| | CCACTTGCCGGCATTTGCGTGGCCCTGCTGCTGTCCCTGATCATCACACTGATC | |
| | tgctaccacaggagccgaaagcgtgtttgcaaatgtcccaggccgctagtcaga | |
| | caggaaggcaagcccagaccttcagagaaaattgtgGGTTCAGGGGCTACCAAC | |
| | TTTTCCCTCCTTAAGCAGGCCGGAGATGTCGAGGAGAATCCTGGCCCTATGGCG | |
| | TGTAATTGCCAGCTCATGCAGGACACACCTCTGTTGAAATTCCCGTGTCCAAGG | |
| | CTTATCCTGCTGTTCGTGCTCTTGATACGACTCAGCCAGGTGTCAAGCGACGTT | |
| | GATGAGCAGCTGTCTAAGAGCGTGAAGGATAAGGTGTTGCTGCCCTGTCGGTAC | |
| | AATAGCCCACATGAGGACGAGTCAGAGGATCGCATCTATTGGCAGAAACACGAC | |
| | AAAGTGGTGCTGAGCGTGATCGCTGGCAAGCTGAAAGTTTGGCCCGAGTACAAG | |
| | AACCGGACACTGTACGACAATACCACATACTCCCTGATTATTCTGGGGCTCGTG | |
| | CTCTCCGATAGAGGCACTTATAGCTGCGTTGTACAGAAGAAGGAAAGAGGGACT | |
| | TATGAGGTTAAACACCTTGCTCTGGTGAAGCTGAGTATCAAGGCTGACTTCTCC | |
| | ACGCCAAATATAACCGAATCAGGAAATCCTAGTGCCGATACTAAACGCATCACT | |
| | TGTTTTGCCAGTGGTGGGTTCCCCAAACCGAGATTCTCTTGGCTGGAAAACGGA | |
| | AGGGAGTTGCCCGGCATTAACACCACCATTTCTCAAGACCCCGAATCCGAGCTT | |
| | TACACCATTAGCAGCCAACTTGACTTCAACACAACACGGAACCACACCATCAAG | |
| | TGTCTGATCAAGTATGGCGACGCACATGTCAGTGAGGATTTCACATGGGAGAAA | |
| | CCACCCGAAGATCCTCCAGACTCCAAGAACACTCTCGTGCTGTTTGGTGCAGGA | |
| | TTTGGAGCCGTAATAACCGTAGTGGTCATTGTCGTCATCATCAAGTGCTTCTGC | |
| | AAACACCGTTCTTGCTTTCGACGCAATGAAGCCTCTAGGGAAACAAACAACTCT | |
| | CTGACTTTTGGCCCTGAAGAAGCCCTGGCAGAGCAAACGGTCTTTCTGTAG | |
| RPL18 peptide | KILTFDRL | 283 |
| | AAGATCCTGACCTTCGACAGGCTC | 284 |

Preparation of Fetal Bovine Serum from which Exosomes are Removed

After 10 mL of inactivated FBS and 2 mL of a 50% poly(ethylene glycol) 10,000 solution (manufactured by Sigma-Aldrich. #81280) were stirred at 4° C., for 2 hours, exosomes were precipitated under centrifugation conditions of 1.500×g, 4° C., and 30 minutes, and supernatant was collected to obtain fetal bovine serum from which the exosomes were removed.

### [Example 1] Antigen-Presenting Extracellular Vesicles Containing MHC Class I Molecules and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with two plasmids (pCAG vectors encoding sc-Trimer-CD81 and CD63-IL-2, respectively) at the same time using polyethylenimine “Max” (manufactured by Polysciences Inc.) according to the manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. A supernatant was collected, and the supernatant was centrifuged at 10,000 g for 30 minutes. Then the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 1 (FIG. 2A). The concentration of the extracellular vesicles was measured according to the manufacturer's instructions using a BCA protein assay kit (manufactured by Thermo Fisher Scientific Inc.).

### [Example 2] Antigen-Presenting Extracellular Vesicles Containing MHC Class I Molecules, T-Cell Costimulatory Molecules, and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with the three plasmids (pCAG vectors encoding sc-Trimer-CD81, CD80-CD9, and CD63-IL-2, respectively) prepared above at the same time using polyethylenimine “Max” (manufactured by Polysciences Inc.) according to the manufacturer's instructions. The medium was 35 replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, a supernatant was collected, and then the supernatant was centrifuged at 300 g for 5 minutes after being passed through a 0.22 μm filter. A supernatant was collected, and the supernatant was centrifuged at 2,000 g for 20 minutes. A supernatant was collected, and the supernatant was centrifuged at 10,000 g for 30 After supernatant was collected and the supernatant was centrifuged at minutes. 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 2 (FIG. 2B). The concentration of the antigen-presenting extracellular vesicles was measured according to the manufacturer's instructions using a BCA protein assay kit (manufactured by Thermo Fisher Scientific Inc.).

### [Example 3] Antigen-Presenting Extracellular Vesicles 1 Containing MHC Class II Molecules, T-Cell Costimulator; Molecules, and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with the four plasmids (pCAG vectors encoding sc-Dimer-CD81, an MHC class IIα chain, CD80-CD9, and CD63-IL-2, respectively) prepared above at the same time using polyethylenimine “Max” (manufactured by Polysciences Inc.) according to the manufacturer's instructions. The medium was replaced 3 hours after the transfection, and 24 hours after the transfection, the medium was replaced with a Dulbecco's modified Eagle medium to which 2% fetal bovine serum from which exosomes were removed and penicillin/streptomycin were added. 72 hours after the transfection, a supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. Then, supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 3 (FIG. 2C). The concentration of the antigen-presenting extracellular vesicles was measured according to the manufacturer's instructions using a BCA protein assay kit (manufactured by Thermo Fisher Scientific Inc.).

### [Example 4] Antigen-Presenting Extracellular Vesicles 2 Containing MHC Class II Molecules, T-Cell Costimulatory Molecules, and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with five plasmids (pCAG vectors encoding sc-Dimer-CD81, an MHC class IIα chain, CD80-CD9. TGF-β-MFGE8, and CD63-IL-2, respectively) at the same time using polyethylenimine “Max” (manufactured by Polysciences Inc.) according to the manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After a supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 4 (FIG. 2D). The concentration of the antigen-presenting extracellular vesicles was measured according to the manufacturer's instructions using a BCA protein assay kit (manufactured by Thermo Fisher Scientific Inc.).

### [Example 5] Antigen-Presenting Extracellular Vesicles 3 Containing MHC Class II Molecules, T-Cell Costimulatory Molecules, and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with the four plasmids (pCAG vectors encoding sc-Dimer-CD81, an MHC class IIα chain. CD80-CD9, and CD81-IL-4, respectively) prepared above at the same time using polyethylenimine “Max” (manufactured by Polysciences Inc.) according to the manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, a supernatant was removed, and the pellets suspended in 100 μL of PBS were used as antigen-presenting extracellular vesicles of Example 5 (FIG. 2E). The concentration of the antigen-presenting extracellular vesicles was measured according to the manufacturer's instructions using a BCA protein assay kit (manufactured by Thermo Fisher Scientific Inc.).

### [Reference Example 1] Control Extracellular Vesicles

HEK293T cells were seeded in a cell culture dish and cultured in a Dulbecco's modified Eagle medium to which 2% fetal bovine serum and penicillin/streptomycin were added. The medium was replaced with cells at about 50% confluence, and after 24 hours, the medium was replaced with a Dulbecco's modified Eagle medium to which 2% fetal bovine exosomes-removed serum and penicillin/streptomycin were added. 48 hours after the replacement with the medium from which exosomes were removed, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After a supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, a supernatant was removed, and the pellets suspended in 100 μL of PBS were used as extracellular vesicles of Reference Example 1. The concentration of the antigen-presenting extracellular vesicles was measured according to the manufacturer's instructions using a BCA protein assay kit (manufactured by Thermo Fisher Scientific Inc.).

### [Reference Example 2] Extracellular Vesicles Containing MHC Class I Molecules in Membrane

HEK293T cells were seeded in a cell culture dish and cultured in a Dulbecco's modified Eagle medium to which 2% fetal bovine serum and penicillin/streptomycin were added. Cells at about 50% confluence were transfected with a plasmid (a pCAG vector encoding sc-Trimer-CD81) using polyethylenimine “Max” (manufactured by Polysciences Inc.) according to manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After a supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as extracellular vesicles of Reference Example 2. The concentration of the extracellular vesicles was measured according to the manufacturer's instructions using a BCA protein assay kit (manufactured by Thermo Fisher Scientific Inc.).

### [Reference Example 3] Extracellular Vesicles Containing T-Cell Costimulatory Molecules in Membrane

HEK293T cells were seeded in a cell culture dish and cultured in a Dulbecco's modified Eagle medium to which 2% fetal bovine serum and penicillin/streptomycin were added. Cells at about 50% confluence were transfected with a plasmid (a pCAG vector encoding CD80-CD9) using polyethylenimine “Max” (manufactured by Polysciences Inc.) according to manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as extracellular vesicles of Reference Example 3. The concentration of the extracellular vesicles was measured according to the manufacturer's instructions using a BCA protein assay kit (manufactured by Thermo Fisher Scientific Inc.).

### [Reference Example 4] Extracellular Vesicles Containing T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with a plasmid (a pCAG vector encoding CD63-IL-2) using polyethylenimine “Max” (Polysciences Inc.) according to manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as extracellular vesicles of Reference Example 4. The concentration of the extracellular vesicles was measured according to the manufacturer's instructions using a BCA protein assay kit (manufactured by Thermo Fisher Scientific Inc.).

### [Reference Example 5] Extracellular Vesicles Containing MHC Class I Molecules and T-Cell Costimulatory Molecules in Membrane

HEK293T cells were seeded in a cell culture dish and cultured in a Dulbecco's modified Eagle medium to which 2% fetal bovine serum and penicillin/streptomycin were added. Cells at about 50% confluence were transfected with two plasmids (pCAG vectors encoding sc-Trimer-CD81 and CD80-CD9, respectively) at the same time using polyethylenimine “Max” (manufactured by Polysciences Inc.) according to the manufacturer's instructions. The medium was replaced 3 hours after the transfection, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After a supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as extracellular vesicles of Reference Example 5. The concentration of the extracellular vesicles was measured according to the manufacturer's instructions using a BCA protein assay kit (manufactured by Thermo Fisher Scientific Inc.).

### Test Example 1-1: Flow Cytometry Analysis of Fusion Protein Contained in Membrane of Extracellular Vesicle

The antigen-presenting extracellular vesicles of Example 2 were immunostained by a PS Capture (trademark) exosome flow cytometry kit (manufactured by FUJIFILM Wako Pure Chemical Corporation) according to the manufacturer's instruction. Antibodies used for staining are as follows (staining time: 15 minutes, temperature: 4° C.). After the staining, expression of each fusion protein was detected with a flow cytometer FACSCantoll (manufactured by BD Biosciences).
APC-conjugated anti-mouse H-2KbOVA complex antibody (25-D1.16, manufactured by Biolegend, Inc.)
PE-conjugated anti-mouse CD80 antibody (16-10A1, manufactured by Biolegend, Inc.)
Brilliant Violet421-conjugated anti-mouse IL-2 antibody (JES6-5H4, manufactured by Biolegend, Inc.)

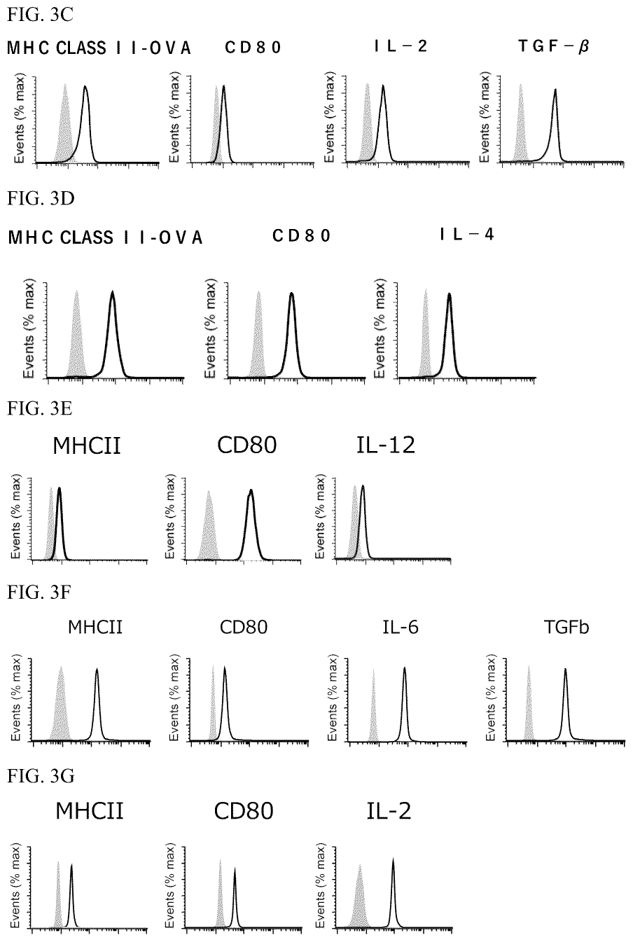

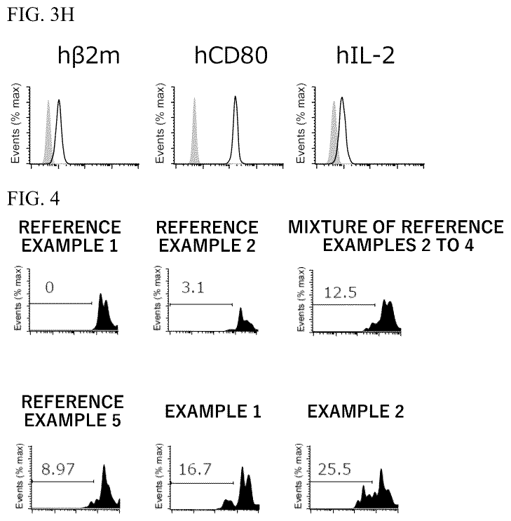

The results are illustrated in FIG. 3A.

### Results

The results of Test Example 1-1 show that MHC class I molecules presenting OVA antigens, CD80, and IL-2 were contained in the membrane of the antigen-presenting extracellular vesicle of Example 2 (FIG. 3A).

### Test Example 1-2: Flow Cytometry Analysis of Fusion Protein Contained in Membrane of Extracellular Vesicle

The antigen-presenting extracellular vesicles of Example 3 were immunostained by a PS Capture (trademark) exosome flow cytometry kit (manufactured by FUJIFILM Wako Pure Chemical Corporation) according to the manufacturer's instruction. The antibodies used for the staining are as follows. After the staining, expression of each fusion protein was detected with a flow cytometer FACSCantoII (manufactured by BD Biosciences).
APC-conjugated anti-mouse IL-2 antibody (JES6-5H4, manufactured by Biolegend, Inc.)
PE-conjugated anti-mouse CD80 antibody (16-10A1, manufactured by Biolegend, Inc.)
APC-Cy7-conjugated anti-mouse I-A/I-E antibody (M5/114.15.2, manufactured by Biolegend, Inc.)

The results are illustrated in FIG. 3B.

### Results

The results of Test Example 1-2 show that MHC class II molecules presenting OVA antigens, CD80, and IL-2 were contained in the membrane of the antigen-presenting extracellular vesicle of Example 3 (FIG. 3B).

### Test Example 1-3: Flow Cytometry Analysis of Fusion Protein Contained in Membrane of Extracellular Vesicle

The antigen-presenting extracellular vesicles of Example 4 were immunostained by a PS Capture (trademark) exosome flow cytometry kit (manufactured by FUJIFILM Wako Pure Chemical Corporation) according to the manufacturer's instruction. The antibodies used for the staining are as follows. After the staining, expression of each fusion protein was detected with a flow cytometer FACSCantoII (manufactured by BD Biosciences).
APC-conjugated anti-mouse IL-2 antibody (JES6-5H4, manufactured by Biolegend, Inc.)
PE-conjugated anti-mouse CD80 antibody (16-10A1, manufactured by Biolegend, Inc.)
APC-Cy7-conjugated anti-mouse I-A/I-E antibody (M5/114.15.2, manufactured by Biolegend, Inc.)
APC-conjugated anti-mouse LAP (TGF-β1) antibody (TW7-16B4, manufactured by Biolegend, Inc.)

The results are illustrated in FIG. 3C.

### Results

The results of Test Example 1-3 show that MHC class II molecules presenting OVA antigens, CD80, IL-2, and TGF-β1 were contained in the membrane of the antigen-presenting extracellular vesicle of Example 4 (FIG. 3C).

### Test Example 1-4: Flow Cytometry Analysis of Fusion Protein Contained in Membrane of Extracellular Vesicle

The antigen-presenting extracellular vesicles of Example 5 were immunostained by a PS Capture (trademark) exosome flow cytometry kit (manufactured by FUJIFILM Wako Pure Chemical Corporation) according to the manufacturer's instruction. The antibodies used for the staining are as follows. After the staining, expression of each fusion protein was detected with a flow cytometer FACSCantoII (manufactured by BD Biosciences).
Alexa Fluor 488-conjugated anti-mouse IL-4 antibody (11B11, manufactured by Biolegend, Inc.)
PE-conjugated anti-mouse CD80 antibody (16-10A1, manufactured by Biolegend, Inc.)
APC-Cy7-conjugated anti-mouse I-A/I-E antibody (M5/114.15.2, manufactured by Biolegend, Inc.)

The results are illustrated in FIG. 3D.

### Results

The results of Test Example 1-4 show that MHC class II molecules presenting OVA antigens, CD80, and IL-4 were contained in the membrane of the antigen-presenting extracellular vesicle of Example 5 (FIG. 3D).

### Test Example 2: Experiment on Activation of OVA-Specific CD8-Positive T Cells (OT-1 T Cells) In Vitro by Antigen-Presenting Extracellular Vesicles

The following test was conducted in vitro to determine whether the antigen-presenting extracellular vesicles activate antigen-specific CD8-positive T cells.

Lymph nodes extracted from an OT-1 mouse, which was an OVA-reactive TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 1 or 2 (final concentration: 3 μg/mL), a mixture of three types of the extracellular vesicles of Reference Examples 2 to 4 (final concentration of each of the three types of the extracellular vesicles: 3 μg/mL), or the extracellular vesicles of Reference Examples 1, 2, or 5 (final concentration: 3 μg/mL) were added, culture was performed in a 96 well round bottom plate for 3 days, and then, immunostaining was performed. Antibodies used for staining are as follows (staining time: 15 minutes, temperature: 4° C.). After the staining, a luminescence intensity of CellTrace Violet as a cell proliferation assay reagent in the OT-1 T cells was detected with a flow cytometer FACSCantoll (manufactured by BD Biosciences).
APC-conjugated anti-mouse CD8 antibody (53-6.7, manufactured by Biolegend, Inc.)
PE-conjugated anti-mouse TCR Vb5.1,5.2 antibody (MR9-4, manufactured by Biolegend, Inc.)

The results are illustrated in FIG. 4.

### Results

The results of Test Example 2 show that the antigen-presenting extracellular vesicles of Examples 1 and 2 remarkably differentiated and/or proliferated antigen-specific CD8-positive T cells in comparison with the mixture of the three types of the extracellular vesicles of Reference Examples 2 to 4 or the extracellular vesicles of Reference Examples 1, 2, and 5 (FIG. 4).

### Test Example 3: Experiment on Activation of OVA-Specific CD8-Positive T Cells (OT-1 T Cells) In Vivo by Antigen-Presenting Extracellular Vesicles

The following test was conducted in vivo to determine whether the antigen-presenting extracellular vesicles activate antigen-specific CD8-positive T cells.

Lymph nodes were extracted from an OT-1 mouse, which was OVA-reactive TCR transgenic mouse, and the same lymphocyte suspension as that of Test Example 2 was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day, a mixture (IL-2/anti-IL-2 antibody complex) of 50 μg of the antigen-presenting extracellular vesicles of Example 2 or the extracellular vesicles of Reference Example 1, or 1.5 μg of IL-2 (manufactured by Biolegend, Inc.) and 50 μg of anti-mouse IL-2 antibodies (S4B6-1, manufactured by Bio X Cell) was transferred to from the tail vein into a CD45.1/CD45.2 congenic mouse. 4 days after cell transfer, lymph nodes were extracted from the recipient mouse, and a lymphocyte suspension was prepared and immunostained. Antibodies used for staining are as follows (staining time: 15 minutes, temperature: 4° C.). After the staining, a luminescence intensity of CellTrace Violet as a cell proliferation assay reagent in the transferred OT-1 T cells and wild-type CD8T cells was detected with a flow cytometer FACSCantoII (manufactured by BD Biosciences).
PE-Cy 7-conjugated anti-mouse CD8 antibody (53-6.7, manufactured by Biolegend, Inc.)
PE-conjugated anti-mouse TCR Vb5.1,5.2 antibody (MR9-4, manufactured by Biolegend, Inc.)
FITC-conjugated anti-mouse CD45.1 antibody (A20, manufactured by Biolegend, Inc.)
APC-conjugated anti-mouse CD45.2 antibody (104, manufactured by Biolegend, Inc.)

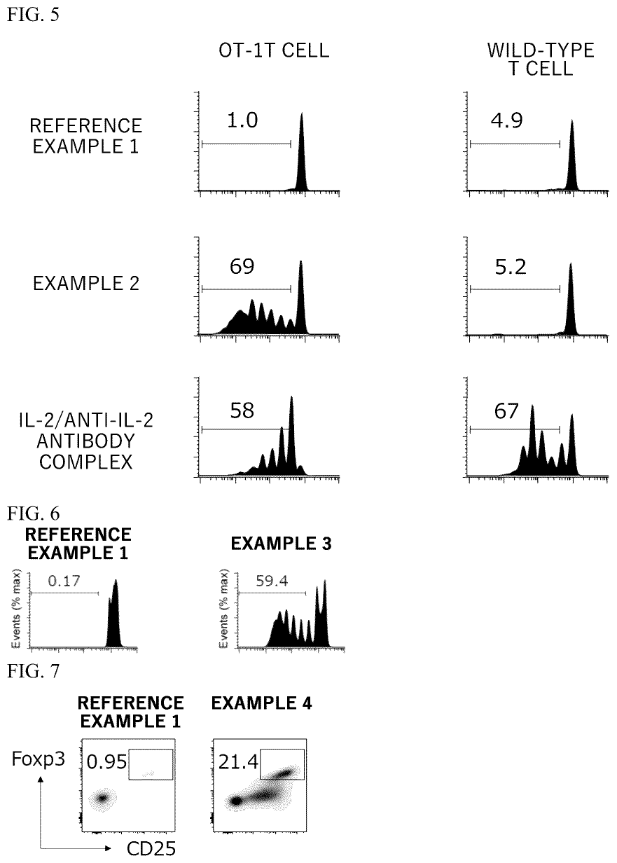

The results are illustrated in FIG. 5.

### Results

The results of Test Example 3 show that the antigen-presenting extracellular vesicles of Example 2 hardly activated other CD8-positive T cells (antigen-non-specific CD8-positive T cells) and remarkably differentiated and/or proliferated antigen-specific CD8-positive T cells in vivo in comparison with the extracellular vesicles of Reference Example 1 (FIG. 5). In addition, it is possible that serious side effects such as cytokine storm are low because the antigen-presenting extracellular vesicles of Example 2 hardly activate other CD8-positive T cells (antigen-non-specific CD8-positive T cells) in comparison with the IL-2/anti-IL-2 antibody complex (FIG. 5).

### Test Example 4: Experiment on Activation of OVA-Specific CD4-Positive T Cells (OT-2 T Cells) In Vitro by Antigen-Presenting Extracellular Vesicles

The following test was conducted in vitro to determine whether the antigen-presenting extracellular vesicles activate antigen-specific CD4-positive T cells.

Lymph nodes extracted from an OT-2 mouse, which was an OVA-reactive CD4TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 3 or the extracellular vesicles of Reference Example 1 were added so that the final concentration was 10 μg/mL, and culture was performed in a 96 well round bottom plate for 4 days. After 4 days, the cells were recovered and immunostained. Antibodies used for staining are as follows (staining time: 15 minutes, temperature: 4° C.). After the staining, a luminescence intensity of CellTrace Violet as a cell proliferation assay reagent in the OT-2 T cells was detected with a flow cytometer FACSCantoII (manufactured by BD Biosciences).
PE-Cy 7-conjugated anti-mouse CD4 antibody (RM4-5, manufactured by Biolegend, Inc.)
APC-conjugated anti-mouse TCR Vb5.1,5.2 antibody (MR9-4, manufactured by Biolegend, Inc.)

The results are illustrated in FIG. 6.

### Results

The results of Test Example 4 show that the antigen-presenting extracellular vesicles of Example 3 remarkably differentiated and/or proliferated antigen-specific CD4 T cells in comparison with the extracellular vesicles of Reference Example 1 (FIG. 6).

### Test Example 5: Experiment on Differentiation Induction of OVA-Specific CD4-Positive T Cells (OT-2 T Cells) In Vitro into Regulatory T Cells by Antigen-Presenting Extracellular Vesicles

The following test was conducted in vitro to determine whether the antigen-presenting extracellular vesicles induce differentiation of antigen-specific CD4-positive T cells into regulatory T cells (Treg).

Lymph nodes extracted from an OT-2 mouse, which was an OVA-reactive CD4TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 4 or the extracellular vesicles of Reference Example 1 were added so that the final concentration was 10 μg/mL, and culture was performed in a 96 well round bottom plate for 4 days. After 4 days, the cells were recovered, and extracellular immunostaining was performed. Antibodies used for staining are as follows (staining time: 15 minutes, temperature: 4° C.). After the extracellular staining, intracellular immunostaining was performed using True-Nuclear Transcription Factor Buffer Set (manufactured by Biolegend, Inc.) and anti-mouse FOXP3 antibodies according to the manufacturer's instructions. After the intracellular staining, expression of CD25 molecules and FOXP3 molecules as markers of regulatory T cells on the OT-2 T cells was detected with a flow cytometer FACSCantoII (manufactured by BD Biosciences).
PE-conjugated anti-mouse CD25 antibody (PC61, manufactured by Biolegend, Inc.)
PE-Cy7-conjugated anti-mouse CD4 antibody (RM4-5, manufactured by Biolegend, Inc.)
APC-conjugated anti-mouse TCR Vb5.1,5.2 antibody (MR9-4, manufactured by Biolegend, Inc.)
Alexa Fluor 488-conjugated anti-mouse FOXP3 antibody (MF-14, manufactured by Biolegend, Inc.)

The results are illustrated in FIG. 7.

### Results

The results of Test Example 5 show that the antigen-presenting extracellular vesicles of Example 4 induced differentiation of the antigen-specific CD4-positive T cells into regulatory T cells (preferably, regulatory T cells expressing Foxp3) in comparison with the extracellular vesicles of Reference Example 1 (FIG. 7).

### Test Example 6: Experiment on Differentiation Induction of OVA-Specific CD4-Positive T Cells (OT-2 T Cells) In Vitro into Th2T Cells by Antigen-Presenting Extracellular Vesicles

The following test was conducted in vitro to determine whether antigen-presenting extracellular vesicles induce differentiation of antigen-specific CD4-positive T cells into Th2T cells.

Lymph nodes extracted from an OT-2 mouse, which was an OVA-reactive CD4TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 3 or 5 or the extracellular vesicles of Reference Example 1 were added so that the final concentration was 10 μg/mL, and culture was performed in a 96 well round bottom plate for 4 days. After 4 days, the cells were recovered, and extracellular immunostaining was performed. Antibodies used for staining are as follows (staining time: 15 minutes, temperature: 4° C.). After the extracellular staining, intracellular immunostaining was performed using True-Nuclear Transcription Factor Buffer Set (manufactured by Biolegend, Inc.) and anti-GATA3 antibodies according to the manufacturer's instructions. After the intracellular staining, a luminescence intensity of CellTrace Violet as a cell proliferation assay reagent in the OT-2 T cells and expression of GATA3 as a marker of Th2T cells were detected with a flow cytometer FACSCantoII (manufactured by BD Biosciences).
PE-conjugated anti-mouse TCR Vb5.1,5.2 antibody (MR9-4, manufactured by Biolegend, Inc.)
PE-Cy 7-conjugated anti-mouse CD4 antibody (RM4-5, manufactured by Biolegend, Inc.)
APC-conjugated anti-GATA3 antibody (16E10A23, manufactured by Biolegend, Inc.)

The results are illustrated in FIG. 8.

### Results

The results of Test Example 6 show that the antigen-presenting extracellular vesicles of Examples 3 and 5 induced differentiation of antigen-specific CD4-positive T cells into Th2 cells in vitro in comparison with the extracellular vesicles of Reference Example 1 (FIG. 8). The Th2 cells secrete cytokines such as IL-4 or IL-5, activate differentiation of naive B cells that recognize the same antigen, and promote induction of antigen-specific IgE production (that is, activation of humoral immunity).

### [Example 6] Antigen-Presenting Extracellular Vesicles 4 Containing MHC Class II Molecules, T-Cell Costimulatory Molecules, and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with the four plasmids (pCAG vectors encoding sc-Dimer-CD81-IL-12p40, an MHC class IIα chain, CD80)-CD9, and IL-12p35, respectively) prepared above at the same time using polyethylenimine “Max” (manufactured by Polysciences Inc.) according to the manufacturer's instructions. 3 to 12 hours after the transfection, the medium was replaced, and 24 hours after the transfection, the medium was replaced with a Dulbecco's modified Eagle medium to which 2% fetal bovine exosomes-removed serum and penicillin/streptomycin were added. 72 hours after the transfection, a supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular 30) vesicles of Example 6. The concentration of the antigen-presenting extracellular vesicles was measured according to the manufacturer's instructions using a BCA protein assay kit (manufactured by Thermo Fisher Scientific Inc.).

### Test Example 1-5: Flow Cytometry Analysis of Fusion Protein Contained in Membrane of Extracellular Vesicle

The antigen-presenting extracellular vesicles of Example 6 were immunostained by a PS Capture (trademark) exosome flow cytometry kit (manufactured by FUJIFILM Wako Pure Chemical Corporation) according to the manufacturer's instruction. The antibodies used for the staining are as follows. After the staining, expression of each fusion protein was detected with a flow cytometer FACSCantoII (manufactured by BD Biosciences).
Alexa Fluor 488-conjugated anti-mouse I-A/I-E antibody M5/114.15.2, manufactured by Biolegend, Inc.)
PE-conjugated anti-mouse IL-12 antibody (C15.6, manufactured by Biolegend, Inc.)
The results of APC-conjugated anti-mouse CD80 antibody (16-10A1, manufactured by Biolegend, Inc.) are illustrated in FIG. 3E.

### Results

The results of Test Example 1-5 show that MHC class II molecules presenting OVA antigens, CD80, and functional IL-12 were contained in the membrane of the antigen-presenting extracellular vesicle of Example 6 (FIG. 3E).

### [Example 7] Antigen-Presenting Extracellular Vesicles 5 Containing MHC Class II Molecules, T-Cell Costimulatory Molecules, and T-Cell Stimulatory Cytokines in Membrane

HEK293T cells were seeded in a cell culture dish and cultured in a Dulbecco's modified Eagle medium to which 2% fetal bovine serum and penicillin/streptomycin were added. Cells at about 50% confluence were transfected with the five plasmids (pCAG vectors encoding sc-Dimer-CD81, an MHC class IIα chain, CD80-CD9, CD81-IL-6, and TGF-β-MFGE8, respectively) prepared above at the same time using polyethylenimine “Max” (manufactured by Polysciences Inc.) according to the manufacturer's instructions. 3 to 12 hours after the transfection, the medium was replaced, and 24 hours after the transfection, the medium was replaced with a Dulbecco's modified Eagle medium to which 2% fetal bovine exosomes-removed serum and penicillin/streptomycin were added. 72 hours after the transfection, a supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 7. The concentration of the antigen-presenting extracellular vesicles was measured according to the manufacturer's instructions using a BCA protein assay kit (manufactured by Thermo Fisher Scientific Inc.).

### Test Example 1-6: Flow Cytometry Analysis of Fusion Protein Contained in Membrane of Extracellular Vesicle

The antigen-presenting extracellular vesicles of Example 7 were immunostained by a PS Capture (trademark) exosome flow cytometry kit (manufactured by FUJIFILM Wako Pure Chemical Corporation) according to the manufacturer's instruction. The antibodies used for the staining are as follows. After the staining, expression of each fusion protein was detected with a flow cytometer FACSCantoII (manufactured by BD Biosciences).
FITC-conjugated anti-mouse CD80 antibody (16-10A1, manufactured by Biolegend, Inc.)
PE-conjugated anti-mouse IL-6 antibody (MP5-20F3, manufactured by Biolegend, Inc.)
APC-Cy7-conjugated anti-mouse I-A/I-E antibody (M5/114.15.2, manufactured by Biolegend, Inc.)
APC-conjugated anti-mouse LAP (TGF-β1) antibody (TW7-16B4, manufactured by Biolegend, Inc.)

The results are illustrated in FIG. 3F.

### Results

The results of Test Example 1-6 show that MHC class II molecules presenting OVA antigens, CD80, IL-6, and TGFb were contained in the membrane of the antigen-presenting extracellular vesicle of Example 7 (FIG. 3F).

### [Example 8] Establishment of Cell Strain Stably Expressing MHC Class I Molecules, T-Cell Costimulatory Molecules, and T-Cell Stimulatory Cytokines and Preparation of Antigen-Presenting Extracellular Vesicles

PLAT-A cells were seeded in a cell culture dish and cultured in a Dulbecco's modified Eagle medium to which 2% fetal bovine serum and penicillin/streptomycin were added. Cells at about 50% confluence were transfected with a pMX vector encoding CD80-CD9 or sc-Trimer-CD81-IL-2 using polyethylenimine “Max” (manufactured by Polysciences Inc.) according to manufacturer's instructions. 12 hours after the transfection, the medium was replaced, and 60 hours after the transfection, a supernatant was collected and centrifuged at 300 g for 5 minutes. The collected supernatant was used as virus particles. HEK293 cells were seeded in a cell culture dish and cultured in a Dulbecco's modified Eagle medium to which 2% fetal bovine serum and penicillin/streptomycin were added. A DOTAP transfection reagent (Roche) was added to viral particles in which the CD80-CD9 adjusted above was incorporated into the cells at about 50% confluence according to the manufacturer's instructions, and the mixture was added to HEK293 cells. The cells to which the viral particles were added were centrifuged at 2.500 rpm for 3 minutes. 24 hours after the transfection, the medium was replaced, and 1 week after the transfection. CD80-positive cells were sorted with FACSMelody (manufactured by BD Biosciences). The sorted CD80-positive cells were cultured for 1 week, and the cultured cells were seeded in a dish and cultured in a Dulbecco's modified Eagle medium to which 2% fetal bovine serum and penicillin/streptomycin were added. A DOTAP transfection reagent was added to viral particles in which the sc-Trimer-CD81-IL-2 prepared above was incorporated into the cells at about 50% confluence according to the manufacturer's instructions, and the mixture was added to CD80-positive HEK293 cells. The cells to which the viral particles were added were centrifuged at 2.500 rpm for 3 minutes. 24 hours after the transfection, the medium was replaced, and 1 week after the transfection. CD80-positive and MHCI-positive cells were sorted with FACSMelody (manufactured by BD Biosciences). The sorted cells were used as stable expression cells. The stable expression cells were seeded in a dish and cultured in a Dulbecco's modified Eagle medium to which 2% fetal bovine serum and penicillin/streptomycin were added. The supernatant of the cells at about 50% confluence was replaced with a Dulbecco's modified Eagle medium to which 2% fetal bovine exosomes-removed and penicillin/streptomycin were added. 72 hours after the medium replacement, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 8.

### Test Example 1-7: Flow Cytometry Analysis of Fusion Protein Contained in Membrane of Extracellular Vesicle

The antigen-presenting extracellular vesicles of Example 8 were immunostained by a PS Capture (trademark) exosome flow cytometry kit (manufactured by FUJIFILM Wako Pure Chemical Corporation) according to the manufacturer's instruction. Antibodies used for staining are as follows (staining time: 15 minutes, temperature: 4° C.). After the staining, expression of each fusion protein was detected with a flow cytometer FACSCantoII (manufactured by BD Biosciences).
PE-conjugated anti-mouse H-2KbOVA complex antibody (25-D1.16, manufactured by Biolegend, Inc.)
FITC-conjugated anti-mouse CD80 antibody (16-10A1, manufactured by Biolegend, Inc.)
APC-conjugated anti-mouse IL-2 antibody (JES6-5H4, manufactured by Biolegend, Inc.)

The results are illustrated in FIG. 3G.

### Results

The results of Test Example 1-7 show that MHC class I molecules presenting OVA antigens, CD80, and IL-2 were contained in the membrane of the antigen-presenting extracellular vesicle of Example 8 (FIG. 3G).

### [Example 9] Antigen-Presenting Extracellular Vesicles Containing HLA Class I Molecules, Human T-Cell Costimulatory Molecules, and Human T-Cell Stimulatory Cytokines in Membrane

HEK293T cells in which B2m was deleted were seeded in a cell culture dish and cultured in a Dulbecco's modified Eagle medium to which 2% fetal bovine serum and penicillin/streptomycin were added. Cells at about 50% confluence were transfected with the two plasmids (pCAG vectors encoding HLAsc-Trimer-human CD81, human CD80-human CD9, and CD63-IL2, respectively) prepared above at the same time using polyethylenimine “Max” (manufactured by Polysciences Inc.) according to the manufacturer's instructions. 3 to 12 hours after the transfection, the medium was replaced, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as human antigen-presenting extracellular vesicles of Example 9. The concentration of the antigen-presenting extracellular vesicles was measured according to the manufacturer's instructions using a BCA protein assay kit (manufactured by Thermo Fisher Scientific Inc.).

By using SARS-COV2sc-Trimer-hCD81 instead of hsc-Trimer-hCD81, it is possible to prepare human antigen-presenting extracellular vesicles presenting antigen-presenting MHC molecules presenting SARS-COV2 peptides as antigens, hCD80, and hIL-2 on a surface thereof.

### Test Example 1-8: Flow Cytometry Analysis of Fusion Protein Contained in Membrane of Extracellular Vesicle

The antigen-presenting extracellular vesicles of Example 9 were immunostained by a PS Capture (trademark) exosome flow cytometry kit (manufactured by FUJIFILM Wako Pure Chemical Corporation) according to the manufacturer's instruction. Antibodies used for staining are as follows (staining time: 15 minutes, temperature: 4° C.). After the staining, expression of each fusion protein was detected with a flow cytometer FACSCantoII (manufactured by BD Biosciences).
APC-conjugated anti-human IL-2 antibody (MQ1-17H12, manufactured by Biolegend, Inc.)
PE-conjugated anti-human CD80 antibody (2D10, manufactured by Biolegend, Inc.)
APC-conjugated anti-human β2m antibody (2M2, manufactured by Biolegend, Inc.)

The results are illustrated in FIG. 3H.

### Results

The results of Test Example 1-8 show that MHC class I molecules presenting WTI antigens, hCD80, and hIL-2 were contained in the membrane of the antigen-presenting extracellular vesicle of Example 9 (FIG. 3H).

### Test Example 7: Experiment on Differentiation Induction of OVA-Specific CD4-Positive T Cells (OT-2 T Cells) In Vitro into Th1T Cells by Antigen-Presenting Extracellular Vesicles

The following test was conducted in vitro to determine whether the antigen-presenting extracellular vesicles induce differentiation of antigen-specific CD4-positive T cells into Th1T cells.

Lymph nodes extracted from an OT-2 mouse, which was an OVA-reactive CD4TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 3 or 6 or the extracellular vesicles of Reference Example 1 were added so that the final concentration was 10 μg/mL, and culture was performed in a 96 well round bottom plate for 4 days. After 4 days, the cells were recovered, and extracellular immunostaining was performed. Antibodies used for staining are as follows (staining time: 15 minutes, temperature: 4° C.). After the extracellular staining, intracellular immunostaining was performed using True-Nuclear Transcription Factor Buffer Set (manufactured by Biolegend, Inc.) and anti-T-bet antibodies according to the manufacturer's instructions. After the intracellular staining, a luminescence intensity of CellTrace Violet as a cell proliferation assay reagent in the OT-2 T cells and expression of T-bet as a marker of Th1T cells were detected with a flow cytometer FACSCantoII (manufactured by BD Biosciences).
PerCP/Cy5.5-conjugated anti-mouse TCRVa2 antibody (B20.1, manufactured by Biolegend, Inc.)
APC-Cy 7-conjugated anti-mouse CD4 antibody (RM4-5, manufactured by Biolegend, Inc.)
PE-conjugated anti-T-bet antibody (4B10, manufactured by Biolegend, Inc.)

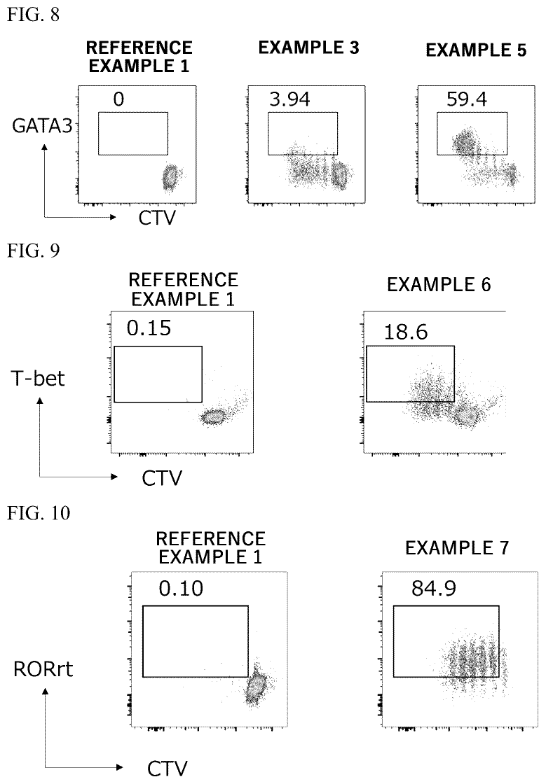

The results are illustrated in FIG. 9.

### Results

The results of Test Example 7 show that the antigen-presenting extracellular vesicles of Example 6 induced differentiation of antigen-specific CD4-positive T cells into Th1 cells in vitro in comparison with the extracellular vesicles of Reference Example 1 (FIG. 9). The Th1 cells produce IFN-γ, IL-2, or the like, and promote activation of macrophages and cytotoxic T cells that destroy pathogen cells, virus-infected cells, cancer cells, and the like (that is, activation of cellular immunity).

### Test Example 8: Experiment on Differentiation Induction of OVA-Specific CD4-Positive T Cells (OT-2 T Cells) In Vitro into Th17T Cells by Antigen-Presenting Extracellular Vesicles

The following test was conducted in vitro to determine whether the antigen-presenting extracellular vesicles induce differentiation of antigen-specific CD4-positive T cells into Th17T cells.

Lymph nodes extracted from a mouse obtained by mating an OVA-reactive CD4TCR transgenic mouse and an RORrt-GFP mouse were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 7 or the extracellular vesicles of Reference Example 1 were added so that the final concentration was 10 μg/mL, and culture was performed in a 96 well round bottom plate for 4 days. After 4 days, the cells were recovered, and extracellular immunostaining was performed. Antibodies used for staining are as follows (staining time: 15 minutes, temperature: 4° C.). After the intracellular staining, a luminescence intensity of CellTrace Violet as a cell proliferation assay reagent in the OT-2 T cells and expression of RORrt as a marker of Th17T cells were detected with a flow cytometer FACSCantoII (manufactured by BD Biosciences).
APC-conjugated anti-mouse TCRVa2 antibody (B20.1, manufactured by Biolegend, Inc.)
PE-Cy7-conjugated anti-mouse CD4 antibody (RM4-5, manufactured by Biolegend, Inc.)
PE-conjugated anti-mouse TCR Vb5.1,5.2 antibody (MR9-4, manufactured by Biolegend, Inc.)

The results are illustrated in FIG. 10.

### Results

The results of Test Example 8 show that the antigen-presenting extracellular vesicles of Example 7 induced differentiation of antigen-specific CD4-positive T cells into Th17 cells in vitro in comparison with the extracellular vesicles of Reference Example 1 (FIG. 10). Unlike the Th1 or Th2 cells, the Th17 cells produce inflammatory cytokines such as IL-17, IL-21, IL-22, and TNF-α to induce inflammation, promote recruitment and proliferation of neutrophils and monocytes, and contribute to infection defense of fungi (including pathogenic fungi such as candida, Staphylococcus aureus, and Streptococcus pyogenes).

### Test Example 9: Experiment on Activation of OVA-Specific CD8-Positive T Cells (OT-1 T Cells) In Vitro by Antigen-Presenting Extracellular Vesicles Obtained by Purification of Stable Cell Strain

The following test was conducted in vitro to determine whether the antigen-presenting extracellular vesicles obtained by purification of a stable cell line activate antigen-specific CD8-positive T cells.

Lymph nodes extracted from an OT-1 mouse, which was an OVA-reactive TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, the antigen-presenting extracellular vesicles of Example 1 or 8 or the extracellular vesicles of Reference Example 1 were added so that the final concentration was 9 μg/mL, and culture was performed in a 96 well round bottom plate for 4 days. After the culture in the 96 well round bottom plate for 3 days, immunostaining was performed. Antibodies used for staining are as follows (staining time: 15 minutes, temperature: 4° C.). After the staining, a luminescence intensity of CellTrace Violet as a cell proliferation assay reagent in the OT-1 T cells was detected with a flow cytometer FACSCantoII (manufactured by BD Biosciences).
APC-conjugated anti-mouse CD8 antibody (53-6.7, manufactured by Biolegend, Inc.)
PE-conjugated anti-mouse TCR Vb5.1,5.2 antibody (MR9-4, manufactured by Biolegend, Inc.)

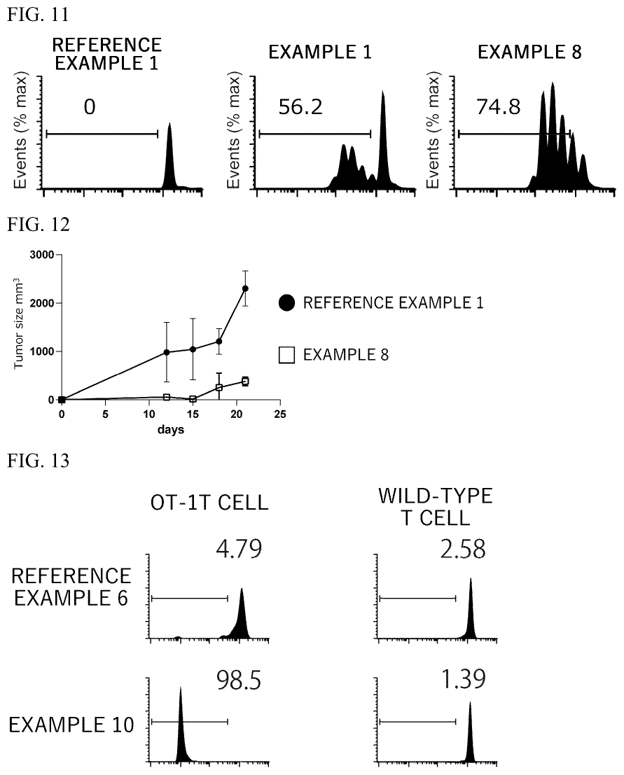

The results are illustrated in FIG. 11.

### Results

The results of Test Example 9 show that the antigen-presenting extracellular vesicles of Examples 1 and 8 remarkably proliferated antigen-specific CD8-positive T cells in comparison with the extracellular vesicles of Reference Example 1 (FIG. 11). This indicates that not only the extracellular vesicles in which the constitutional requirement (A) exemplified by sc-Trimer-CD81 and the constitutional requirement (B) exemplified by CD81-IL-2 are present as different proteins, but also the extracellular vesicles in which a fusion protein having both functions, which is exemplified by sc-Trimer-CD81-IL-2, is present, exhibit equivalent effects on T cells.

### Test Example 10: Experiment for Evaluating Anti-Tumor Effect by Antigen-Presenting Extracellular Vesicles

In order to determine whether the antigen-presenting extracellular vesicles prepared from the stable cell strain have an anti-tumor effect, 1×10⁵ B16 melanoma cells expressing OVA were subcutaneously ingested in a CD45.1/CD45.2 congenic mouse, and 1×10⁵ OT-1T cells were transferred after 3 days. After 1 day, 4 days, and 7 days after the OT-1T cells were transferred, 50 μg of the antigen-presenting extracellular vesicles of Example 8 or the extracellular vesicles of Reference Example 1 were injected from the tail vein of the recipient mouse, and the size of the B16 melanoma cells was observed.

### Results

The results of Test Example 10 show that the antigen-presenting extracellular vesicles of Example 8 remarkably suppressed proliferation of B16 melanoma cells in comparison with the extracellular vesicles of Reference Example 1 (FIG. 12). This indicates that not only the extracellular vesicles in which the constitutional requirement (A) exemplified by sc-Trimer-CD81 and the constitutional requirement (B) exemplified by CD81-IL-2 are present as different proteins, but also the extracellular vesicles in which a fusion protein having both functions, which is exemplified by sc-Trimer-CD81-IL-2, is present, exhibit equivalent medicinal effects.

### [Example 10] Preparation of mRNA Expressing sc-Trimer-CD81-IL-2 Fusion Protein

A pET-15b vector encoding sc-Trimer-CD81-IL-2 was linearized using EagI, the linearized vector was purified using a FastGene Gel/PCR extraction kit (NIPPON Genetics Co., Ltd.), and transcription, capping, and poly A addition were performed on the purified vector in vitro using T7 mScript Standard mRNA Production System (manufactured by CELLSCRIPT, LLC) according to the manufacturer's instructions. The synthesized mRNA was used as RNA producing antigen-presenting cells and antigen-presenting extracellular vesicles of Example 10 (FIG. 1N).

### Reference Example 6: Preparation of mRNA Expressing CD63-AkaLuc Fusion Protein

A pET-15b vector encoding CD63-AkaLuc was linearized using EcoRI, the linearized vector was purified using a FastGene Gel/PCR extraction kit (NIPPON Genetics Co., Ltd.), and transcription, capping, and poly A addition were performed on the purified vector in vitro using T7 mScript Standard mRNA Production System (manufactured by CELLSCRIPT, LLC) according to the manufacturer's instructions. The synthesized mRNA was used as a control RNA of Reference Example 6 (FIG. 1O).

### Test Example 11: Experiment on Activation of OVA-Specific CD8-Positive T Cells (OT-1 T Cells) In Vivo by mRNA Expressing sc-Trimer-CD81-IL-2 Fusion Protein

The following test was conducted in vivo to determine whether mRNA expressing a sc-Trimer-CD81-IL-2 fusion protein activated antigen-specific CD8-positive T cells.

Lymph nodes extracted from an OT-1 mouse, which was an OVA-reactive TCR transgenic mouse, were disrupted on a 100 μm filter to obtain a lymph node cell suspension. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day, 10 μg of mRNA of Example 10 or Reference Example 6 was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, the spleen was extracted from the recipient mouse, and a lymphocyte suspension was prepared and immunostained. Antibodies used for staining are as follows (staining time: 15 minutes, temperature: 4° C.). After the staining, a luminescence intensity of CellTrace Violet as a cell proliferation assay reagent in the transferred OT-1 T cells and wild-type CD8T cells was detected with a flow cytometer FACSCantoll (manufactured by BD Biosciences).
PE-Cy 7-conjugated anti-mouse CD8 antibody (53-6.7, manufactured by Biolegend, Inc.)
PE-conjugated anti-mouse TCR Vb5.1,5.2 antibody (MR9-4, manufactured by Biolegend, Inc.)
FITC-conjugated anti-mouse CD45.1 antibody (A20, manufactured by Biolegend, Inc.)
APC-conjugated anti-mouse CD45.2 antibody (104, manufactured by Biolegend, Inc.)

The results are illustrated in FIG. 13.

### Results

The results of Test Example 11 show that mRNA of Example 10 hardly activated other CD8-positive T cells (antigen-non-specific CD8-positive T cells) and remarkably differentiated and/or proliferated antigen-specific CD8-positive T cells in vivo in comparison with mRNA of Reference Example 6 (FIG. 13). Although not particularly limited to the principle, it is understood that the polynucleotide according to the present invention was introduced into any cell in the CD45.1/CD45.2 congenic mouse, a sc-Trimer-CD81-IL-2 fusion protein was expressed on the membrane surface of the cell and/or the membrane surface of the extracellular vesicle secreted from the cell to produce antigen-presenting cells and/or antigen-presenting extracellular vesicles, the produced antigen-presenting cells and/or antigen-presenting extracellular vesicles are brought into contact with OT-1 T cells, and thereby OVA-reactive CD8T cells were proliferated. This indicates that an effect equivalent to the effect of the T cell activation of the antigen-presenting cells and/or the antigen-presenting extracellular vesicles shown in Test Examples 1 to 10 can also be obtained in vivo by the polynucleotide for producing antigen-presenting extracellular vesicles.

### Test Example 12: Experiment on Activation of Intrinsic OVA-Reactive T Cells by mRNA Expressing sc-Trimer-CD81-IL-2 Fusion Protein

10 μg of mRNA of Example 10 or Reference Example 6 was mixed with an in vivo-jetRNA transfection reagent (Polyplus-transfection SA) according to the manufacturer's instructions, and the mixture was transferred from a tail vein of a C57BL/6 mouse. 4 days after transfer, the spleen was extracted from the recipient mouse, a lymphocyte suspension was prepared, and OVA-reactive T cells were immunostained with a tetramer according to the manufacturer's instructions. The antibodies used for the staining are as follows. After the staining, tetramer-positive cells were detected with a flow cytometer FACSCantoII (manufactured by BD Biosciences).
APC-conjugated H-2Kb OVA Tetramer (Tetramer Shop ApS).
PE-conjugated H-2Kb OVA Tetramer (Tetramer Shop ApS)
Brilliant Violet421-conjugated anti-mouse CD8 antibody (53-6.7, manufactured by Biolegend, Inc.)

### Results

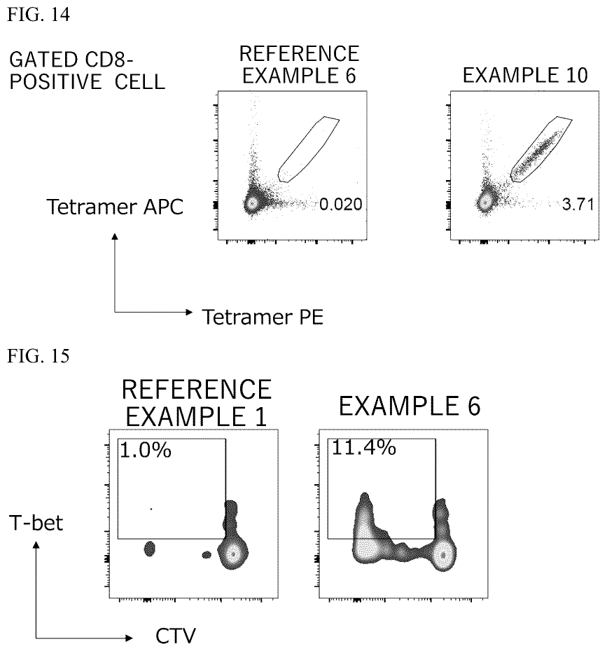

The results of Test Example 12 show that mRNA of Example 10 remarkably proliferated the OVA-reactive CD8T cells that were intrinsically present in comparison with mRNA of Reference Example 6 (FIG. 14). Although not particularly limited to the principle, it is understood that the polynucleotide according to the present invention was introduced into any cell in the C57BL/6 congenic mouse, a sc-Trimer-CD81-IL-2 fusion protein is expressed on the membrane surface of the cell and/or the membrane surface of the extracellular vesicle secreted from the cell to produce antigen-presenting cells and/or antigen-presenting extracellular vesicles, the produced antigen-presenting cells and/or antigen-presenting extracellular vesicles contact with intrinsic T cells, and thereby OVA-reactive CD8T cells were proliferated. This indicates that an effect equivalent to the effect of the antigen-presenting extracellular vesicle as a pharmaceutical shown in Test Examples 1 to 10 can also be obtained in vivo by the polynucleotide for producing antigen-presenting cells and/or antigen-presenting extracellular vesicles.

### Test Example 13: Experiment on Differentiation Induction of OVA-Specific CD4-Positive T Cells (OT-2 T Cells) In Vivo into Th1T Cells by Antigen-Presenting Extracellular Vesicles

Lymph nodes extracted from an OT-2 mouse, which was an OVA-reactive TCR transgenic mouse, were disrupted on a 100 μm filter to obtain a lymph node cell suspension. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. 1 day and 4 days after lymphocyte transfer, 50 μg of the extracellular vesicles of Example 6 or Reference Example 1 were subcutaneously transferred to a CD45.1/CD45.2 congenic mouse. 7 days after cell transfer, the spleen was extracted from the recipient mouse, and a lymphocyte suspension was prepared and immunostained. Antibodies used for staining are as follows (staining time: 15 minutes, temperature: 4° C.). After the staining, a luminescence intensity of CellTrace Violet as a cell proliferation assay reagent in the transferred OT-2 T cells and wild-type CD4T cells was detected with a flow cytometer FACSCantoll (manufactured by BD Biosciences).
APC-Cy7-conjugated anti-mouse CD4 antibody (RM4-5, manufactured by Biolegend, Inc.)
PerCP/Cy5.5-conjugated anti-mouse TCRVa2 antibody (B20.1, manufactured by Biolegend, Inc.)
FITC-conjugated anti-mouse CD45.1 antibody (A20, manufactured by Biolegend, Inc.)
PE-Cy 7-conjugated anti-mouse CD45.2 antibody (104, manufactured by Biolegend, Inc.)
PE-conjugated anti-T-bet antibody (4B10, manufactured by Biolegend, Inc.)

### Results

The results of Test Example 13 show that the extracellular vesicles of Example 6 hardly activated other CD4-positive T cells (antigen-non-specific CD4-positive T cells) and remarkably differentiated antigen-specific CD4-positive T cells into Th1 in vivo in comparison with the extracellular vesicles of Reference Example 1 (FIG. 15).

### Test Example 14: Experiment for Evaluating Anti-Tumor Effect by Antigen-Presenting Extracellular Vesicles

In order to determine whether the extracellular vesicles of Example 6 had an anti-tumor effect, 1×10⁵ B16 melanoma cells expressing OVA were subcutaneously ingested in a CD45.1/CD45.2 congenic mouse, and 5×10⁵ OT-2T cells were transferred after 1 day. 1 day, 4 days, and 7 days after the OT-2T cell transfer, 50 μg of the antigen-presenting extracellular vesicles of Example 6 or the extracellular vesicles of Reference Example 1 were subcutaneously transferred from the recipient mouse, and the size of the B16 melanoma cells was observed.

### Results

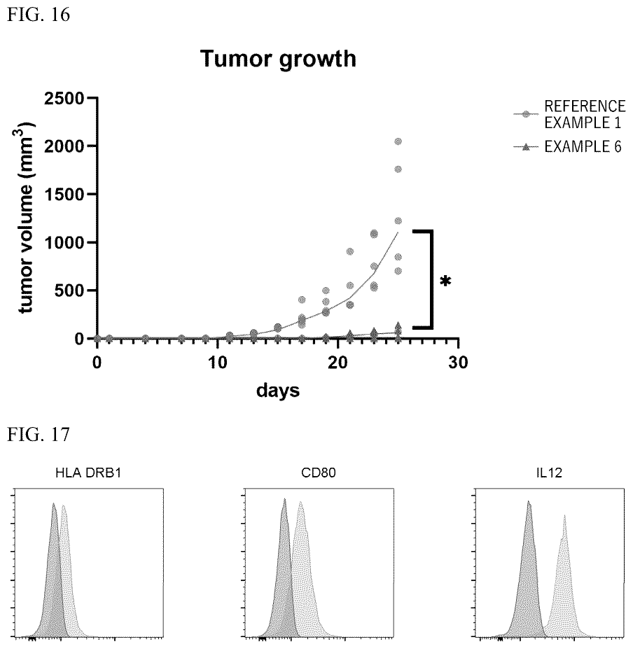

The results of Test Example 14 show that the antigen-presenting extracellular vesicles of Example 6 remarkably suppressed proliferation of B16 melanoma cells in comparison with the extracellular vesicles of Reference Example 1 (FIG. 16).

### [Example 11] Antigen-Presenting Extracellular Vesicles Containing HLA Class II Molecules, Human T-Cell Costimulatory Molecules, and Human T-Cell Stimulatory Cytokines in Membrane

HEK293T cells in which B2m was deleted were seeded in a cell culture dish and cultured in a Dulbecco's modified Eagle medium to which 2% fetal bovine serum and penicillin/streptomycin were added. Cells at about 50% confluence were transfected with the two plasmids (pCAG vectors encoding HLADR-1sc-TPI1-human CD81, human CD80-human CD9), and human IL-12sc-MFGe8, respectively) prepared above at the same time using polyethylenimine “Max” (manufactured by Polysciences Inc.) according to the manufacturer's instructions. 3 to 12 hours after the transfection, the medium was replaced, and 24 hours after the transfection, the medium was replaced with a Dulbecco's modified Eagle medium to which 2% fetal bovine exosomes-removed serum and penicillin/streptomycin were added. 72 hours after the transfection, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as human antigen-presenting extracellular vesicles of Example 11 (FIG. 2J). The concentration of the antigen-presenting extracellular vesicles was measured according to the manufacturer's instructions using a BCA protein assay kit (manufactured by Thermo Fisher Scientific Inc.).

### Test Example 15: Flow Cytometry Analysis of Fusion Protein Contained in Membrane of Extracellular Vesicle

The antigen-presenting extracellular vesicles of Example 11 were immunostained by a PS Capture (trademark) exosome flow cytometry kit (manufactured by FUJIFILM Wako Pure Chemical Corporation) according to the manufacturer's instructions. The antibodies used for the staining are as follows.

After the staining, expression of each fusion protein was detected with a flow cytometer FACSCantoII (manufactured by BD Biosciences).
PE-conjugated anti-human HLA-DRB1 antibody (NFLD.D2, manufactured by Biolegend, Inc.)
PE-conjugated anti-human CD80 antibody (W17149D, manufactured by Biolegend, Inc.)
APC-conjugated anti-human IL-12 antibody (C11.5, manufactured by Biolegend, Inc.)

The results are illustrated in FIG. 17.

### Results

The results of Test Example 15 show that MHC class II molecules (that is, HLA-DR) presenting TPI-1 antigens, neoantigens, CD80, and IL-12 were contained in the membrane of the antigen-presenting extracellular vesicle of Example 11 (FIG. 17).

### Test Example 16: Experiment on Differentiation Induction of TPI-1-Specific Human CD4-Positive T Cells In Vitro into Th1T Cells by Antigen-Presenting Extracellular Vesicles

The following test was conducted in vitro to determine whether the human antigen-presenting extracellular vesicles induce differentiation of antigen-specific CD4-positive T cells into Th1T cells. PlatA cells (retroviral package cells) were transfected with a TPI-1 peptide-specific TCR (T cell receptor) and a pMXs vector encoding a fluorescent protein Venus using polyethylenimine “Max” (manufactured by Polysciences Inc.). The medium was changed 3 to 12 hours after transfection, supernatant was collected 48 and 72 hours after transfection, and the supernatant was passed through a 0.22 μm filter to prepare retroviral supernatant expressing a TPI-1 specific TCR. Peripheral blood was collected from an HLA-DR1-positive recipient, peripheral blood mononuclear cells were separated using Ficol, 2.0×10⁶ peripheral blood mononuclear cells were suspended in 200 μL of an RPMI 1640 medium to which 10% fetal bovine serum, 50 μM 2-mercaptoethanol, and penicillin/streptomycin were added, human IL-2 and 25 μl of dynabeads were mixed so that the final concentration was 20 ng/mL, and stimulation was performed in a 6 well plate for 24 hours. The viral particles prepared above were infected with RetroNectin (TAKARA) as instructed by the manufacturer to prepare human T cells simultaneously expressing a TPI-1 peptide-specific TCR and fluorescent protein Venus. The extracellular vesicles of Example 11 or Reference Example 1 were added to the prepared human T cells, and the cells were cultured in a 96 well round bottom plate for 7 days. After 7 days, the cells were recovered, and extracellular immunostaining was performed. Antibodies used for staining are as follows (staining time: 15 minutes, temperature: 4° C.). After the extracellular staining, intracellular immunostaining was performed using True-Nuclear Transcription Factor Buffer Set (manufactured by Biolegend, Inc.) and anti-T-bet antibodies according to the manufacturer's instructions. After the intracellular staining, Venus luminescence intensity and expression of Th1T markers T-bet and IFN-γ were detected with a flow cytometer FACSCantoll (manufactured by BD Biosciences).
FITC-conjugated anti-human CD4 antibody (A161A1, manufactured by Biolegend, Inc.)
Brilliant Violet 421-conjugated anti-human IFN-r antibody (4S.B3, manufactured by Biolegend, Inc.)
PE-conjugated anti-T-bet antibody (4B10, manufactured by Biolegend, Inc.)

### Results

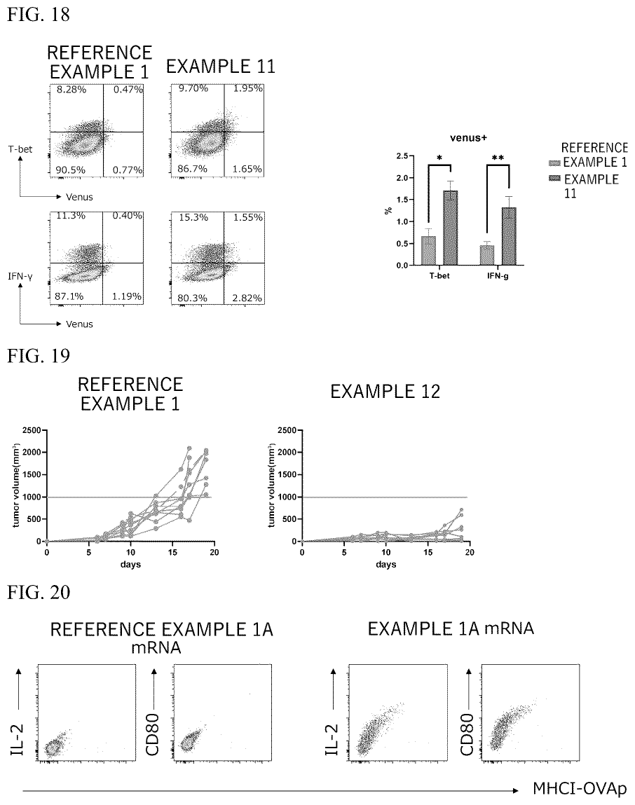

The results of Test Example 16 show that the antigen-presenting extracellular vesicles of Example 11 induced differentiation of TPI-1 peptide antigen-specific CD4-positive T cells into Th1 cells in vitro in comparison with the extracellular vesicles of Reference Example 1 (FIG. 18). The Th1 cells produce IFN-γ, IL-2, or the like, and promote activation of macrophages and cytotoxic T cells that destroy pathogen cells, virus-infected cells, cancer cells, and the like (that is, activation of cellular immunity).

### [Example 12] Establishment of Cell Strain Stably Expressing MHC Class I Molecules. T-Cell Costimulatory Molecules, and T-Cell Stimulatory Cytokines and Preparation of Antigen-Presenting Extracellular Vesicles

PLAT-A cells were seeded in a cell culture dish and cultured in a Dulbecco's modified Eagle medium to which 2% fetal bovine serum and penicillin/streptomycin were added. Cells at about 50% confluence were transfected with a pMX vector encoding CD80-MFG-E8 or sc-Trimer-CD81-IL-2 using polyethylenimine “Max” (manufactured by Polysciences Inc.) according to the manufacturer's instructions. 12 hours after the transfection, the medium was replaced, and 60 hours after the transfection, a supernatant was collected and centrifuged at 300 g for 5 minutes. The collected supernatant was used as virus particles. HEK293 cells were seeded in a cell culture dish and cultured in a Dulbecco's modified Eagle medium to which 2% fetal bovine serum and penicillin/streptomycin were added. A DOTAP transfection reagent (Roche) was added to viral particles in which the CD80-MFG-E8 prepared above was incorporated into the cells at about 50% confluence according to the manufacturer's instructions, and the mixture was added to HEK293 cells. The cells to which the viral particles were added were centrifuged at 2.500 rpm for 3 minutes. 24 hours after the transfection, the medium was replaced, and 1 week after the transfection. CD80-positive cells were sorted with FACSMelody (manufactured by BD Biosciences). The sorted CD80-positive cells were cultured for 1 week, and the cultured cells were seeded in a dish and cultured in a Dulbecco's modified Eagle medium to which 2% fetal bovine serum and penicillin/streptomycin were added. A DOTAP transfection reagent was added to viral particles in which the sc-Trimer-CD81-IL-2 prepared above was incorporated into the cells at about 50% confluence according to the manufacturer's instructions, and the mixture was added to CD80-positive HEK293 cells. The cells to which the viral particles were added were centrifuged at 2.500 rpm for 3 minutes. 24 hours after the transfection, the medium was replaced, and 1 week after the transfection. CD80-positive and MHCI-positive cells were sorted with FACSMelody (manufactured by BD Biosciences). The sorted cells were used as stable expression cells. The stable expression cells were seeded in a dish and cultured in a Dulbecco's modified Eagle medium to which 2% fetal bovine serum and penicillin/streptomycin were added. The supernatant of the cells at about 50% confluence was replaced with a Dulbecco's modified Eagle medium to which 2% fetal bovine exosomes-removed and penicillin/streptomycin were added. 72 hours after the medium replacement, supernatant was collected, and then the supernatant was centrifuged at 300 g for 5 minutes after being passed through a 0.22 μm filter. Supernatant was collected, and the supernatant was centrifuged at 2,000 g for 20 minutes. Supernatant was collected, and the supernatant was centrifuged at 10,000 g for 30 minutes. After supernatant was collected and the supernatant was centrifuged at 100,000 g for 2 hours, the supernatant was removed, and pellets were washed with PBS. After PBS was added to the pellets and the pellets were centrifuged at 100,000 g for 2 hours, supernatant was removed, and the pellets suspended in 100 μL of PBS were used as antigen-presenting extracellular vesicles of Example 12.

### Test Example 17: Experiment for Evaluating Anti-Tumor Effect by Antigen-Presenting Extracellular Vesicles

In order to determine whether the antigen-presenting extracellular vesicles obtained by purification of a stable cell strain have an anti-tumor effect. 1×10⁵ EL-4 cells expressing OVA were subcutaneously ingested in a C57BL/6 mouse. 1 day. 4 days, and 7 days after the EL-4 cell ingestion. 50 μg of the antigen-presenting extracellular vesicles of Example 12 or the extracellular vesicles of Reference Example 1 were transferred from the tail vein of the recipient mouse, and the size of the EL-4 cells was observed.

### Results

The results of Test Example 17 show that the antigen-presenting extracellular vesicles of Example 12 remarkably suppressed proliferation of EL-4 cells in comparison with the extracellular vesicles of Reference Example 1 (FIG. 19).

### [Example 1A] Preparation of mRNA Encoding sc-Trimer-T2A-IL-2-CD8-P2A-CD80

A pET-15b vector encoding sc-Trimer-T2A-IL-2-CD8-P2A-CD80 was linearized using HindIII, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT, LLC) according to the manufacturer's instructions. The synthesized mRNA was used as RNA inducing antigen-presenting cells of Example 1A (FIG. 1P).

### [Reference Example 1A] Preparation of Control mRNA

A pET-15b vector encoding CD81 was linearized using HindIII, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT, LLC) according to the manufacturer's instructions. The synthesized mRNA was used as a control RNA of Reference Example 1A (FIG. 1Q).

### [Reference Example 2A] Preparation of mRNA Encoding OVA

A pET-15b vector encoding OVA was linearized using HindIII, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT, LLC) according to the manufacturer's instructions. The synthesized mRNA was used as a control RNA of Reference Example 2A (FIG. 1R).

### Test Example 1A: Flow Cytometry Analysis of Antigen-Presenting Cells Induced by mRNA

B16 melanoma cells were seeded in a cell culture dish and cultured in a Dulbecco's modified Eagle medium to which 2% fetal bovine serum and penicillin/streptomycin were added. Cells at about 50% confluence were transfected with mRNA of Example 1A and mRNA of Reference Example 1A using TransIT-mRNA Transfection Kit (manufactured by Mirus Bio LLC) according to the manufacturer's instructions. 24 hours after transfection, B16 melanoma cells were collected and immunostained according to the manufacturer's instructions. Antibodies used for staining are as follows (staining time: 15 minutes, temperature: 4° C.). After the staining, expression of each protein was detected with a flow cytometer FACSCantoll (manufactured by BD Biosciences).
PE-conjugated anti-mouse H-2KbOVA complex antibody (25-D1.16, manufactured by Biolegend, Inc.)
FITC-conjugated anti-mouse CD80 antibody (16-10A1, manufactured by Biolegend, Inc.)
APC-conjugated anti-mouse IL-2 antibody (JES6-5H4, manufactured by Biolegend, Inc.)

### Results

The results of Test Example 1A show that the mRNA of Example 1A was transfected into B16 melanoma cells (MO4 cells) expressing OVA and expressed an antigen-MHC I complex, CD80, and IL-2 on the MO4 cells (FIG. 20).

### Test Example 2A: Experiment on Activation of OVA-Specific CD8-Positive T Cells (OT-1 T Cells) In Vitro by Antigen-Presenting Cells

The following test was conducted in vitro to determine whether the antigen-presenting cells activate antigen-specific CD8-positive T cells.

Lymph nodes extracted from an OT-1 mouse, which was an OVA-reactive TCR transgenic mouse, were disrupted on a 100 μm filter to obtain a lymph node cell suspension. The cell suspension was stained using CellTrace Violet (manufactured by Thermo Fisher Scientific Inc.) as a cell proliferation assay reagent according to the manufacturer's instructions. 2×10⁵ stained lymph node cells were suspended in 200 μL of an RPMI1640 medium to which 10% fetal bovine serum, 50 μM 2-mercaptoethanol, and penicillin/streptomycin were added, 1×10⁴ MO4 cells transfected with mRNA of Example 1A, Reference Example 1A, or Reference Example 2A were added and cultured in a 96 well round bottom plate for 3 days, and then immunostaining was performed. Antibodies used for staining are as follows (staining time: 15 minutes, temperature: 4° C.). After the staining, a luminescence intensity of CellTrace Violet as a cell proliferation assay reagent in the OT-1 T cells was detected with a flow cytometer FACSCantoII (manufactured by BD Biosciences).
APC-conjugated anti-mouse CD8 antibody (53-6.7, manufactured by Biolegend, Inc.)
PE-conjugated anti-mouse TCR Vb5.1,5.2 antibody (MR9-4, manufactured by Biolegend, Inc.)

### Results

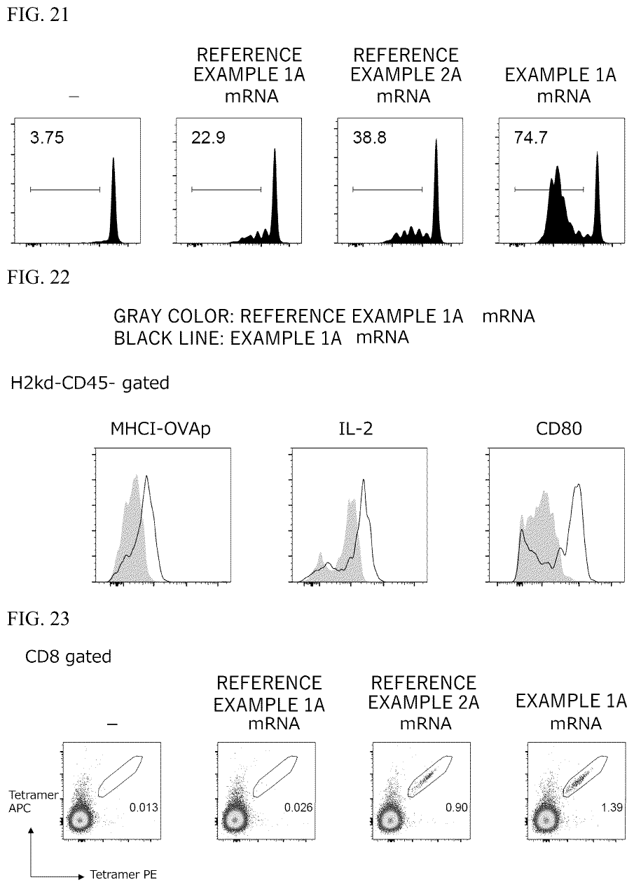

The results of Test Example 2A show that the antigen-presenting cells induced by mRNA of Example 1A remarkably proliferated antigen-specific CD8-positive T cells as compared with the MO4 cells transfected with mRNAs of Reference Examples 1A and 2A (FIG. 21).

### Test Example 3A: Experiment of Converting Melanoma Cells into Antigen-Presenting Cells In Vivo

1×10⁵ B16 melanoma cells expressing OVA were subcutaneously ingested in a nude mouse, and after the volume of the tumor reached about 100 mm³, 9 μg of mRNA was mixed with an in vivo-jetRNA transfection reagent (Polyplus-transfection SA) according to the manufacturer's instructions, and the mixture was administered into the tumor. The next day, the tumor was excised from the recipient mouse, and a tumor suspension was prepared and immunostained according to the manufacturer's instructions. Antibodies used for staining are as follows (staining time: 15 minutes, temperature: 4° C.). After the staining, expression of each protein was detected with a flow cytometer FACSCantoII (manufactured by BD Biosciences).
PE-conjugated anti-mouse H-2KbOVA complex antibody (25-D1.16, manufactured by Biolegend, Inc.)
PE-Cy7-conjugated anti-mouse CD80 antibody (16-10A1, manufactured by Biolegend, Inc.)
APC-conjugated anti-mouse IL-2 antibody (JES6-5H4, manufactured by Biolegend, Inc.)
Pacific Blue-conjugated anti-mouse CD45 antibody (30-F11, manufactured by Biolegend, Inc.)
FITC-conjugated anti-H2-Kd antibody (SF1-1.1, manufactured by Biolegend, Inc.)

### Results

The results of Test Example 3A show that mRNA of Example 1A induced expression of OVAp-MHCI, IL-2-CD8, and CD80 proteins on the membrane surface of the B16 melanoma cell expressing OVA in vivo (FIG. 22). This indicates that some of the melanoma cells were converted into antigen-presenting cells in vivo.

### Test Example 4A: Experiment of Activation of Intrinsic OVA-Reactive T Cells by Antigen-Presenting Cells Induced In Vivo

2 μg of mRNA of Example 1A or each of Reference Examples 1A and 2A was mixed with an in vivo-jetRNA transfection reagent (Polyplus-transfection SA) according to the manufacturer's instructions, and the mixture was transferred from a tail vein of a C57BL/6 mouse. 7 days after transfer, the spleen was extracted from the recipient mouse, a lymphocyte suspension was prepared, and OVA-reactive T cells were immunostained with a tetramer according to the manufacturer's instructions. The antibodies used for the staining are as follows. After the staining, tetramer-positive cells were detected with a flow cytometer FACSCantoII (manufactured by BD Biosciences).
APC-conjugated H-2Kb OVA Tetramer (Tetramer Shop ApS)
PE-conjugated H-2Kb OVA Tetramer (Tetramer Shop ApS)
Brilliant Violet421-conjugated anti-mouse CD8 antibody (53-6.7, manufactured by Biolegend, Inc.)

### Results

The results of Test Example 4A show that mRNA of Example 1A remarkably proliferated the OVA-reactive CD8T cells that were intrinsically present in comparison with mRNAs of Reference Examples 1A and 2A (FIG. 23). Although it is not particularly limited to the principle, it is understood that mRNA according to the present invention was introduced into any cell in a C57BL/6 mouse body, antigen-presenting cells in which OVAp-MHCI, IL-2-CD8, and CD80 proteins were respectively expressed were induced on the membrane surface of the cell, intrinsic T cells were in contact with the antigen-presenting cells, and thereby OVA-reactive CD8T cells were proliferated.

### [Example 2A] Preparation of mRNA Encoding sc-Trimer-T2A-IL-15sa-P2A-CD80

A vector encoding sc-Trimer-T2A-TfR-IL-15sa-P2A-CD80 and containing a T7 promoter, a human α globulin sequence in 3′UTR, and a poly A sequence of a 129 base was linearized using HindIII and purified using a FastGene Gel/PCR extraction kit (NIPPON Genetics Co., Ltd.), and in vitro transcription and capping were performed using HiScribe T7 mRNA Kit with CleanCap Reagent AG (manufactured by New England Biolabs Inc.) according to the manufacturer's instructions. The synthesized mRNA was used as RNA inducing antigen-presenting cells of Example 2A (FIG. 24(a)).

### Test Example 5A: Flow Cytometry Analysis of Antigen-Presenting Cells Induced by mRNA

293T cells were seeded in a cell culture dish and cultured in a Dulbecco's modified Eagle medium to which 2% fetal bovine serum and penicillin/streptomycin were added. Cells at about 50% confluence were transfected with mRNA of Example 2A using TransIT-mRNA Transfection Kit (manufactured by Mirus Bio LLC) according to manufacturer's instructions. 24 hours after transfection, 293T cells were collected and immunostained according to the manufacturer's instructions. Antibodies used for staining are as follows (staining time: 15 minutes, temperature: 4° C.). After the staining, expression of each protein was detected with a flow cytometer FACSCantoII (manufactured by BD Biosciences).
PE-conjugated anti-mouse H-2KbOVA complex antibody (25-D1.16, manufactured by Biolegend, Inc.)
FITC-conjugated anti-mouse CD80 antibody (16-10A1, manufactured by Biolegend, Inc.)
APC-conjugated anti-DYKDDDDK Tag antibody (L5, manufactured by Biolegend, Inc.)

### Results

The results of Test Example 5A show that 293T cells were transfected with mRNA of Example 2A, such that an antigen-MHC I complex, CD80, and IL-15sa were expressed on the 293T cells (FIG. 25).

### Test Example 6A: Experiment of Activation of Intrinsic OVA-Reactive T Cells by Antigen-Presenting Cells Induced In Vivo

5 μg of mRNA of Example 2A or Reference Example 1A was mixed with an in vivo-jetRNA transfection reagent (Polyplus-transfection SA) according to the manufacturer's instructions, and the mixture was transferred from a tail vein of a C57BL/6 mouse. 7 days after transfer, the spleen was extracted from the recipient mouse, a lymphocyte suspension was prepared, and OVA-reactive T cells were immunostained with a tetramer according to the manufacturer's instructions. The antibodies used for the staining are as follows. After the staining, tetramer-positive cells were detected with a flow cytometer FACSCantoII (manufactured by BD Biosciences).
FITC-conjugated anti-mouse CD62L antibody (MEL14, manufactured by Biolegend, Inc.)
APC-conjugated H-2Kb OVAp Tetramer (Tetramer Shop ApS)
PE-conjugated H-2Kb OV Ap Tetramer (Tetramer Shop ApS)
PE/Cyanine7-conjugated anti-mouse CD8 antibody (53-6.7, manufactured by Biolegend, Inc.)
APC/Cyanine7-conjugated anti-mouse CD44 antibody (IM7, manufactured by Biolegend, Inc.)

### Results

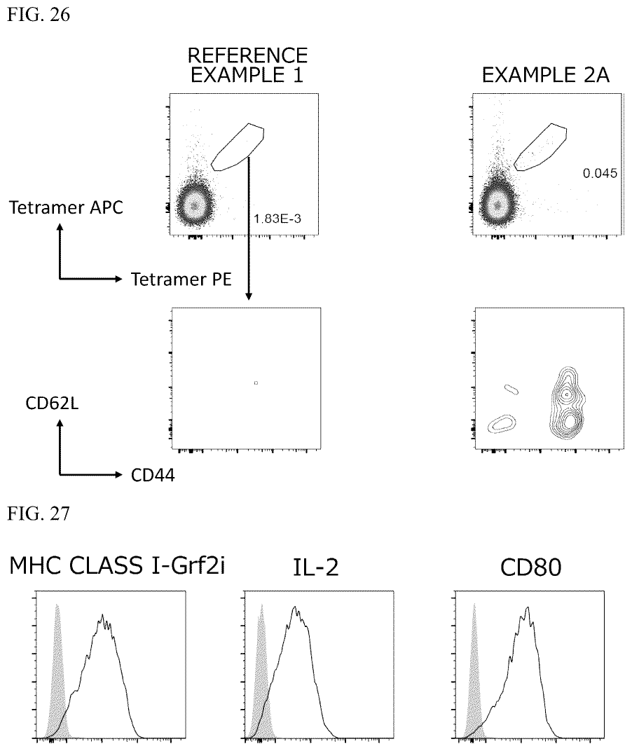

The results of Test Example 6A show that mRNA of Example 2A remarkably proliferated the OVA-reactive CD8T cells that were intrinsically present in comparison with mRNA of Reference Example 1A (FIG. 26). The proliferated cells became an effector memory phenotype of CD44hiCD62low (that is, it means that the proliferated cells can rapidly produce cytokines and make an immune response when exposed to the same antigen again). Although it is not particularly limited to the principle, it is understood that mRNA according to the present invention was introduced into any cell in a C57BL/6 mouse body, antigen-presenting cells in which OVA-MHCI, IL-15sa, and CD80 proteins were respectively expressed were induced on the membrane surface of the cell, intrinsic T cells were in contact with the antigen-presenting cells, and thereby OVA-reactive CD8T cells were proliferated.

### [Example 3A] Preparation of mRNA Encoding sc-Trimer-T2A-IL-2-CD8-P2A-CD80 Presenting Neoantigen

A vector encoding sc-Trimer-T2A-IL-2-CD8-P2A-CD80 presenting a mutated Gtf2i peptide, a neoantigen (cancer antigen) derived from an MC38 colon cancer cell line, instead of an OVA peptide, was linearized using HindIII, the linearized vector was purified using a FastGene Gel/PCR extraction kit (NIPPON Genetics Co., Ltd.), and in vitro transcription, capping, and polyA addition were performed on the purified vector using T7 mScript Standard mRNA Production System (manufactured by CELLSCRIPT, LLC) according to the manufacturer's instructions. The synthesized mRNA was used as RNA inducing antigen-presenting cells of Example 3A (FIG. 24(b)).

### Test Example 7A: Flow Cytometry Analysis of Antigen-Presenting Cells Presenting Neoantigen Induced by mRNA

293T cells were seeded in a cell culture dish and cultured in a Dulbecco's modified Eagle medium to which 2% fetal bovine serum and penicillin/streptomycin were added. Cells at about 50% confluence were transfected with mRNA of Example 3A using TransIT-mRNA Transfection Kit (manufactured by Mirus Bio LLC) according to manufacturer's instructions. 24 hours after transfection, 293T cells were collected and immunostained according to the manufacturer's instructions. Antibodies used for staining are as follows (staining time: 15 minutes, temperature: 4° C.). After the staining expression of each protein was detected with a flow cytometer FACSCantoll (manufactured by BD Biosciences).
PE-conjugated anti-mouse β₂-microglobulin antibody (A16041A, manufactured by Biolegend, Inc.)
FITC-conjugated anti-mouse CD80 antibody (16-10A1, manufactured by Biolegend, Inc.)
APC-conjugated anti-mouse IL-2 antibody (JES6-5H4, manufactured by Biolegend, Inc.)

### Results

The results of Test Example 7A show that 293T cells were transfected with mRNA of Example 3A, such that the neoantigen-MHC I complex, CD80 and IL-2 were expressed on 293T cells (FIG. 27).

### Test Example 8A: Experiment of Activation of Intrinsic Gtf2i-Reactive T Cells by Antigen-Presenting Cells Induced In Vivo

3 μg of mRNA of Example 3A or Reference Example 1A was mixed with an in vivo-jetRNA transfection reagent (Polyplus-transfection SA) according to the manufacturer's instructions, and the mixture was transferred from a tail vein of a C57BL/6 mouse. 7 days after transfer, the spleen was extracted from the recipient mouse, a lymphocyte suspension was prepared, and Gtf2i-reactive T cells were immunostained with a tetramer according to the manufacturer's instructions. The antibodies used for the staining are as follows. After the staining, tetramer-positive cells were detected with a flow cytometer FACSCantoII (manufactured by BD Biosciences).
FITC-conjugated anti-mouse CD62L antibody (MEL14, manufactured by Biolegend, Inc.)
APC-conjugated H-2Kb Gtf2ip Tetramer (Tetramer Shop ApS)
PE-conjugated H-2Kb Gtf2ip Tetramer (Tetramer Shop ApS)
PE/Cyanine7-conjugated anti-mouse CD8 antibody (53-6.7, manufactured by Biolegend, Inc.)
APC/Cyanine7-conjugated anti-mouse CD44 antibody (IM7, manufactured by Biolegend, Inc.)

### Results

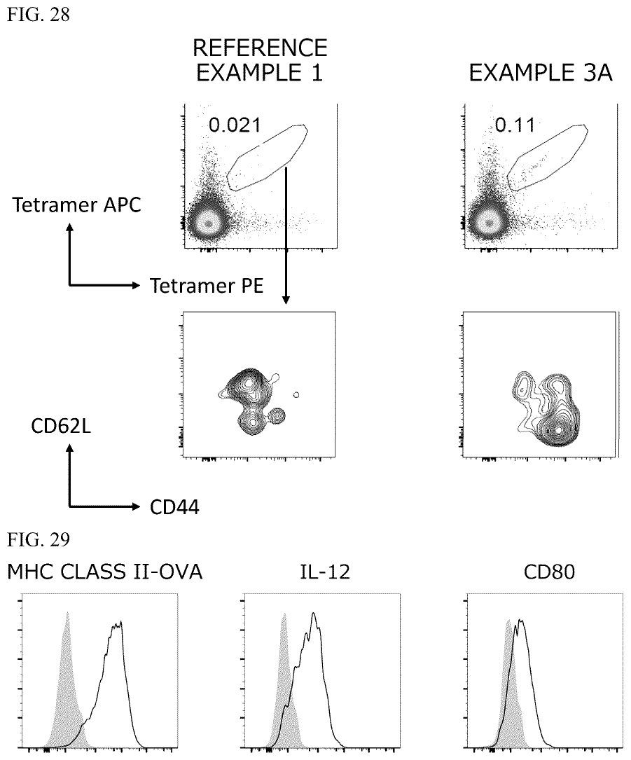

The results of Test Example 8A show that mRNA of Example 3A remarkably proliferated the Gtf2i-reactive CD8T cells that were intrinsically present in comparison with mRNA of Reference Example 1A (FIG. 28). The proliferated cells became an effector memory phenotype of CD44hiCD62low. Although it is not particularly limited to the principle, it is understood that mRNA according to the present invention was introduced into any cell in a C57BL/6 mouse body, antigen-presenting cells in which neoantigen-MHCI, IL-2-CD8, and CD80 proteins were respectively expressed were induced on the membrane surface of the cell, intrinsic T cells were in contact with the antigen-presenting cells, and thereby neoantigen-reactive CD8T cells were proliferated.

### [Example 4A] Preparation of mRNA Encoding OV Ap-MHCIIβ-P2A-MHCIIα-T2A-IL-12sc-CD8-P2A-CD80

A vector encoding OVAp-MHCIIβ-P2A-MHCIIα-T2A-IL-12sc-CD8-P2A-CD80 and containing a T7 promoter, a human α globulin sequence in 3′UTR, and a poly A sequence of a 129 base was linearized using HindIII and purified using a FastGene Gel/PCR extraction kit (NIPPON Genetics Co., Ltd.), and in vitro transcription and capping were performed using HiScribe T7 mRNA Kit with CleanCap Reagent AG (manufactured by New England Biolabs Inc.) according to the manufacturer's instructions. The synthesized mRNA was used as RNA inducing antigen-presenting cells of Example 4A (FIG. 24(c)).

### Test Example 9A: Flow Cytometry Analysis of Antigen-Presenting Cells Induced by mRNA

293T cells were seeded in a cell culture dish and cultured in a Dulbecco's modified Eagle medium to which 2% fetal bovine serum and penicillin/streptomycin were added. Cells at about 50% confluence were transfected with mRNA of Example 4A using TransIT-mRNA Transfection Kit (manufactured by Mirus Bio LLC) according to manufacturer's instructions. 24 hours after transfection, 293T cells were collected and immunostained according to the manufacturer's instructions. Antibodies used for staining are as follows (staining time: 15 minutes, temperature: 4° C.). After the staining, expression of each protein was detected with a flow cytometer FACSCantoII (manufactured by BD Biosciences).
PE-conjugated anti-mouse IL-12/IL23 p40 antibody (25-D1.16, manufactured by Biolegend, Inc.)
FITC-conjugated anti-mouse CD80 antibody (16-10A1, manufactured by Biolegend, Inc.)
APC-conjugated anti-MHC II antibody (M5/114.15.2, manufactured by Biolegend, Inc.)

### Results

The results of Test Example 9A show that 293T cells were transfected with mRNA of Example 4A, such that an OVA-MHC II complex, CD80, and IL-12 were expressed on the 293T cells (FIG. 29).

### Test Example 10A: Experiment of Activation of Intrinsic OVA-Reactive T Cells by Antigen-Presenting Cells Induced In Vivo

The following test was conducted in vivo to determine whether the antigen-presenting cells differentiate antigen-specific CD4-positive T cells into Th1 cells.

Lymph nodes extracted from an OT-II mouse, which was an OVA-reactive TCR transgenic mouse, were disrupted on a 100 μm filter to obtain a lymph node cell suspension. After staining with CellTrace Violet, a cell proliferation assay reagent, was performed, 5×10⁶ CellTrace Violet-stained mixed lymphocyte suspension suspended in PBS was transferred from the tail vein of the CD45.1 congenic mouse. The next day, 10 μg of mRNA of Example 4A or Reference Example 1A or Reference Example 2A was mixed with an in vivo-jetRNA transfection reagent (Polyplus-transfection SA) according to the manufacturer's instructions, and the mixture was transferred from a tail vein to a CD45.1 congenic mouse. 7 days after mRNA transfer, the spleen was extracted from the recipient mouse, and a lymphocyte suspension was prepared and immunostained. Antibodies used for staining are as follows (staining time: 15 minutes, temperature: 4° C.). After the staining, a luminescence intensity of CellTrace Violet as a cell proliferation assay reagent in the transferred OVA-reactive CD4T cells was detected with a flow cytometer FACSCantoII (manufactured by BD Biosciences).
PE-Cy 7-conjugated anti-T-bet antibody (4B10, manufactured by Biolegend, Inc.)
PE-conjugated anti-mouse TCR Vb5.1,5.2 antibody (MR9-4, manufactured by Biolegend, Inc.)
FITC-conjugated anti-mouse CD45.1 antibody (A20, manufactured by Biolegend, Inc.)
APC-conjugated anti-mouse CD45.2 antibody (104, manufactured by Biolegend, Inc.)
APC-Cy 7-conjugated anti-mouse CD4 antibody (RM4-5, manufactured by Biolegend, Inc.)

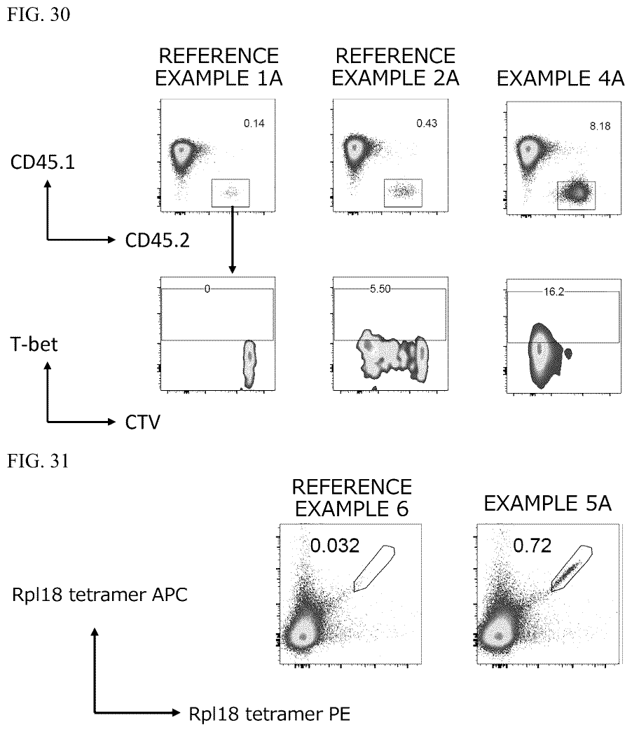

The results are illustrated in FIG. 30.

### Results

The results of Test Example 10A show that mRNA of Example 4A remarkably proliferated the OVA-reactive CD4T cells in comparison with mRNAs of Reference Example 1A and Reference Example 2A (FIG. 30). In addition, some of the proliferated cells differentiated into T-bet-positive Th1 cells. Although it is not particularly limited to the principle, it is understood that mRNA according to the present invention was introduced into any cell in a C57BL/6 mouse body, antigen-presenting cells in which OVA-MHCII, IL-12, and CD80 proteins were respectively expressed were induced on the membrane surface of the cell, and OVA-reactive CD4T cells were in contact with the antigen-presenting cells and then proliferated and differentiated into Th1 cells.

### [Example 5A] Preparation of mRNA Expressing sc-Trimer (RPL18 Peptide)-CD81-IL-2 Fusion Protein

A pET-15b vector encoding sc-Trimer (RPL18 peptide)-CD81-IL-2 presenting a mutated RPL18 peptide, a neoantigen (cancer antigen) derived from an MC38 colon cancer cell line, instead of an OVA peptide, was linearized using EagI, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT, LLC) according to the manufacturer's instructions. The synthesized mRNA was used as RNA producing antigen-presenting cells and antigen-presenting extracellular vesicles of Example 5A.

### Test Example 11A: Experiment on Activation of Intrinsic RPL18 Peptide-Reactive T Cells by mRNA Expressing sc-Trimer (RPL18 Peptide)-CD81-IL-2 Fusion Protein

10 μg of mRNA of Example 5A or Reference Example 6 was mixed with an in vivo-jetRNA transfection reagent (Polyplus-transfection SA) according to the manufacturer's instructions, and the mixture was transferred from a tail vein of a C57BL/6 mouse. 4 days after transfer, the spleen was extracted from the recipient mouse, a lymphocyte suspension was prepared, and RPL18 peptide-reactive T cells were immunostained with a tetramer according to the manufacturer's instructions. The antibodies used for the staining are as follows. After the staining, tetramer-positive cells were detected with a flow cytometer FACSCantoII (manufactured by BD Biosciences).
APC-conjugated H-2Kb RPL18 Tetramer (Tetramer Shop ApS)
PE-conjugated H-2Kb RPL18 Tetramer (Tetramer Shop ApS)
Brilliant Violet421-conjugated anti-mouse CD8 antibody (53-6.7, manufactured by Biolegend, Inc.)

The results of Test Example 11A show that mRNA of Example 5A remarkably proliferated the RPL18-reactive CD8T cells that were intrinsically present in comparison with mRNA of Reference Example 6 (FIG. 31). Although not particularly limited to the principle, it is understood that the polynucleotide according to the present invention is introduced into any cell in the C57BL/6 mouse, a sc-Trimer-CD81-IL-2 fusion protein is expressed on the membrane surface of the cell and/or the membrane surface of the extracellular vesicle secreted from the cell to produce antigen-presenting cells and/or antigen-presenting extracellular vesicles, and the produced antigen-presenting cells and/or antigen-presenting extracellular vesicles contact with intrinsic T cells to proliferate RPL18-reactive CD8T cells. This indicates that an effect equivalent to the effect of the antigen-presenting extracellular vesicle as a pharmaceutical shown in Test Examples 1 to 10 can also be obtained in vivo by the polynucleotide for producing antigen-presenting cells and/or antigen-presenting extracellular vesicles.

### [Example 1B] Polynucleotide for Producing Antigen-Presenting Extracellular Vesicles Containing MHC Class I Molecules and T-Cell Stimulatory Cytokines in Membrane

A pET-15b vector encoding each of sc-Trimer-CD81 and CD63-IL-2 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1 and then used as RNAs for producing the antigen-presenting extracellular vesicles of Example 1B.

### [Example 2B] Polynucleotide for Producing Antigen-Presenting Extracellular Vesicles Containing MHC Class I Molecules, T-Cell Costimulatory Molecules, and T-Cell Stimulatory Cytokines in Membrane

A pET-15b vector encoding each of sc-Trimer-CD81, CD80-CD9, and CD63-IL-2 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1:1 and then used as RNAs for producing the antigen-presenting extracellular vesicles of Example 2B.

### [Reference Example 2B] Polynucleotide for Producing Extracellular Vesicles Containing MHC Class I Molecules in Membrane

A pET-15b vector encoding sc-Trimer-CD81 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNA was used as RNA for producing the antigen-presenting extracellular vesicles of Reference Example 2B.

### [Reference Example 3B] Polynucleotide for Producing Extracellular Vesicles Containing T-cell Costimulatory Molecules in Membrane

A pET-15b vector encoding CD80-CD9 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNA was used as RNA for producing the antigen-presenting extracellular vesicles of Reference Example 3B.

### [Reference Example 4B] Polynucleotide for Producing Extracellular Vesicles Containing T-Cell Stimulatory Cytokines in Membrane

A pET-15b vector encoding CD63-IL-2 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNA was used as RNA for producing the antigen-presenting extracellular vesicles of Reference Example 4B.

### [Reference Example 5B] Polynucleotide for Producing Extracellular Vesicles Containing MHC Class I Molecules and T-Cell Costimulatory Molecules in Membrane

A pET-15b vector encoding each of sc-Trimer-CD81 and CD80-CD9 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1 and then used as RNAs for producing the antigen-presenting extracellular vesicles of Reference Example 5B.

### Test Example 3B: Experiment on Activation of OVA-Specific CD8-Positive T Cells (OT-1 T Cells) In Vivo by Polynucleotide for Producing Antigen-Presenting Extracellular Vesicles

Lymph nodes were extracted from an OT-1 mouse, which was OVA-reactive TCR transgenic mouse, and the same lymphocyte suspension as that of Test Example 2B was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day. 50 μg of mRNA of each of Examples 1A to 5A and Reference Examples 2A to 5A was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNAs of Examples 1B and 2B can remarkably differentiate and/or proliferate antigen-specific CD8-positive T cells in vivo.

### [Example 3B] Polynucleotide for Producing Antigen-Presenting Extracellular Vesicles 1 Containing MHC Class II Molecules. T-Cell Costimulatory; Molecules, and T-Cell Stimulatory Cytokines in Membrane

A pET-15b vector encoding each of sc-Dimer-CD81, an MHC class IIα chain. CD80-CD9, and CD63-IL-2 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1:1:1 and then used as RNAs for producing the antigen-presenting extracellular vesicles of Example 3B.

### Test Example 4B: Experiment on Activation of OVA-Specific CD4-Positive T Cells (OT-2 T Cells) In Vivo by Polynucleotide for Producing Antigen-Presenting Extracellular Vesicles

Lymph nodes were extracted from an OT-2 mouse, which was OVA-reactive CD4TCR transgenic mouse, and the same lymphocyte suspension as that of Test Example 2 was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day. 50 μg of mRNA of Example 3B was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNA of Example 3B can remarkably differentiate and/or proliferate antigen-specific CD4-positive T cells in vivo.

### [Example 4B] Polynucleotide for Producing Antigen-Presenting Extracellular Vesicles 2 Containing MHC Class II Molecules. T-Cell Costimulatory Molecules, and T-Cell Stimulatory Cytokines in Membrane

A pET-15b vector encoding each of sc-Dimer-CD81, an MHC class IIα chain. CD80-CD9. TGF-β-MFGE8, and CD63-IL-2 was linearized, the linearized vector was purified using a FastGene Gel/PCR extraction kit (NIPPON Genetics Co., Ltd.), and in vitro transcription, capping, and polyA addition were performed on the purified vector using T7 mScript Standard mRNA Production System (manufactured by CELLSCRIPT. LLC) according to the manufacturer's instructions. The synthesized mRNAs were mixed at 1:1:1:1:1 and then used as RNAs for preparing the antigen-presenting extracellular vesicles of Example 4B.

### Test Example 5B: Experiment on Activation of OVA-Specific CD4-Positive T Cells (OT-2 T Cells) In Vivo by Polynucleotide for Producing Antigen-Presenting Extracellular Vesicles

Lymph nodes were extracted from an OT-2 mouse, which was OVA-reactive CD4TCR transgenic mouse, and the same lymphocyte suspension as that of Test Example 2B was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day. 50 μg of mRNA of Example 4B was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNA of Example 4B can remarkably differentiate and/or proliferate antigen-specific regulatory T cells in vivo.

### [Example 5B] Polynucleotide for Producing Antigen-Presenting Extracellular Vesicles 3 Containing MHC Class II Molecules, T-Cell Costimulatory Molecules, and T-Cell Stimulatory Cytokines in Membrane

A pET-15b vector encoding each of sc-Dimer-CD81, an MHC class IIα chain. CD80-CD9, and CD81-IL-4 was linearized, the linearized vector was purified using a FastGene Gel/PCR extraction kit (NIPPON Genetics Co., Ltd.), and in vitro transcription, capping, and polyA addition were performed on the purified vector using T7 mScript Standard mRNA Production System (manufactured by CELLSCRIPT. LLC) according to the manufacturer's instructions. The synthesized mRNAs were mixed at 1:1:1:1 and then used as RNAs for preparing the antigen-presenting extracellular vesicles of Example 5B.

### Test Example 6B: Experiment on Activation of OVA-Specific CD4-Positive T Cells (OT-2 T Cells) In Vivo by Polynucleotide for Producing Antigen-Presenting Extracellular Vesicles

Lymph nodes were extracted from an OT-2 mouse, which was OVA-reactive CD4TCR transgenic mouse, and the same lymphocyte suspension as that of Test Example 2 was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day. 50 μg of mRNA of Example 3A or 5A was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNAs of Examples 3B and 5B can remarkably differentiate and/or proliferate antigen-specific Th2 cells in vivo.

### [Example 6B] Polynucleotide for Producing Antigen-Presenting Extracellular Vesicles 4 Containing MHC Class II Molecules, T-Cell Costimulatory Molecules, and T-Cell Stimulatory Cytokines in Membrane

A pET-15b vector encoding each of sc-Dimer-CD81-IL-12p40, an MHC class IIα chain, CD80-CD9, and IL-12p35 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1:1:1 and then used as RNAs for preparing the antigen-presenting extracellular vesicles of Example 6B.

### [Example 7B] Polynucleotide for Producing Antigen-Presenting Extracellular Vesicles 5 Containing MHC Class II Molecules, T-Cell Costimulatory Molecules, and T-Cell Stimulatory Cytokines in Membrane

A pET-15b vector encoding each of sc-Dimer-CD81, an MHC class IIα chain. CD80-CD9, CD81-IL-6, and TGF-β-MFGE8 was linearized, the linearized vector was purified using a FastGene Gel/PCR extraction kit (NIPPON Genetics Co., Ltd.), and in vitro transcription, capping, and polyA addition were performed on the purified vector using T7 mScript Standard mRNA Production System (manufactured by CELLSCRIPT. LLC) according to the manufacturer's instructions. The synthesized mRNAs were mixed at 1:1:1:1:1 and then used as RNAs for preparing the antigen-presenting extracellular vesicles of Example 7B.

### [Example 8B] Polynucleotide for Producing Antigen-Presenting Extracellular Vesicles Containing MHC Class I Molecules. T-Cell Costimulatory Molecules, and T-Cell Stimulatory Cytokines in Membrane

A pET-15b vector encoding each of CD80-CD9 and sc-Trimer-CD81-IL-2 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1 and then used as RNAs for preparing the antigen-presenting extracellular vesicles of Example 8B.

### [Example 9B] Polynucleotide for Producing Antigen-Presenting Extracellular Vesicles Containing HLA Class I Molecules, Human T-Cell Costimulatory Molecules, and Human T-Cell Stimulatory Cytokines in Membrane

A pET-15b vector encoding each of hsc-Trimer-hCD81, hCD80-hCD9, and hCD63-IL2 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1:1 and then used as RNAs for producing the antigen-presenting extracellular vesicles of Example 7A.

### Test Example 7B: Experiment on Activation and Differentiation of OVA-Specific CD4-Positive T Cells (OT-2 T Cells) In Vivo by Polynucleotide for Producing Antigen-Presenting Extracellular Vesicles

Lymph nodes were extracted from an OT-2 mouse, which was OVA-reactive CD4TCR transgenic mouse, and the same lymphocyte suspension as that of Test Example 2 was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day. 50 μg of mRNA of Example 3B or 6B was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNAs of Examples 3B and 6B can remarkably differentiate and/or proliferate antigen-specific Th1 cells in vivo.

### Test Example 8B: Experiment on Activation and Differentiation of OVA-Specific CD4-Positive T Cells (OT-2 T Cells) In Vivo by Polynucleotide for Producing Antigen-Presenting Extracellular Vesicles

Lymph nodes were extracted from an OT-2 mouse, which was OVA-reactive CD4TCR transgenic mouse, and the same lymphocyte suspension as that of Test Example 2 was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day. 50 μg of mRNA of Example 7B was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNA of Example 7B can remarkably differentiate and/or proliferate antigen-specific Th17 cells in vivo.

### Test Example 9B: Experiment on Activation of OVA-Specific CD8-Positive T Cells (OT-1 T Cells) In Vivo by Polynucleotide for Producing Antigen-Presenting Extracellular Vesicles

Lymph nodes were extracted from an OT-1 mouse, which was OVA-reactive TCR transgenic mouse, and the same lymphocyte suspension as that of Test Example 2B was prepared. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. The next day, 50 μg of mRNA of Example 8B was mixed with an in vivo-jetRNA transfection reagent (Polyplus-transfection SA) according to the manufacturer's instructions, and the mixture was transferred from a tail vein to a CD45.1/CD45.2 congenic mouse. 4 days after cell transfer, lymph nodes were extracted from the recipient mouse to prepare a lymphocyte suspension, and various kinds of T cells were detected and quantified by performing immunostaining.

mRNA of Example 8B can remarkably differentiate and/or proliferate antigen-specific CD8-positive T cells in vivo.

### Test Example 10B: Experiment for Evaluating Anti-Tumor Effect by Polynucleotide for Producing Antigen-Presenting Extracellular Vesicles

1×10⁵ B16 melanoma cells expressing OVA were subcutaneously ingested in a CD45.1/CD45.2 congenic mouse, and 1×10⁵ OT-1T cells were transferred after 3 days. 1 day, 4 days, and 7 days after OT-1T cell transfer, 50 μg of mRNA from Example 8B was mixed with an in vivo-jetRNA transfection reagent (Polyplus-transfection SA), the mixture was transferred from a tail vein of a recipient mouse, and the size of B16 melanoma cells was observed.

mRNA of Example 8B can remarkably suppress the proliferation of B16 melanoma cells.

### Test Example 13B: Experiment on Differentiation of OVA-Specific CD4-Positive T Cells (OT-2 T Cells) into Th1T Cells In Vivo by Polynucleotide for Producing Antigen-Presenting Extracellular Vesicles

Lymph nodes extracted from an OT-2 mouse, which was an OVA-reactive TCR transgenic mouse, were disrupted on a 100 μm filter to obtain a lymph node cell suspension. Lymph nodes were similarly extracted from a CD45.1 congenic mouse, and a lymphocyte suspension was prepared. The respective lymphocyte suspensions were mixed at a ratio of 1:1, and the mixture was stained using CellTrace Violet as a cell proliferation assay reagent. 1×10⁷ CellTrace Violet-stained mixed lymphocyte suspension suspended in PBS was transferred from the tail vein of the CD45.1/CD45.2 congenic mouse. 1 day and 4 days after lymphocyte transfer, 50 μg of mRNA of Example 6B or Reference Example 1B was mixed with an in vivo-jetRNA transfection reagent (Polyplus-transfection SA), and the mixture was transferred from a tail vein of a recipient mouse. 7 days after mRNA transfer, the spleen was extracted from the recipient mouse, and a lymphocyte suspension was prepared and immunostained. Antibodies used for staining are as follows (staining time: 15 minutes, temperature: 4° C.). After the staining, a luminescence intensity of CellTrace Violet as a cell proliferation assay reagent in the transferred OT-2 T cells and wild-type CD4T cells was detected with a flow cytometer FACSCantoll (manufactured by BD Biosciences).

### Results

mRNA of Example 6B can differentiate antigen-specific CD4-positive T cells into Th1 cells.

### Test Example 14B: Experiment for Evaluating Anti-Tumor Effect by Polynucleotide for Producing Antigen-Presenting Extracellular Vesicles

In order to determine whether mRNA of Example 6B had an anti-tumor effect. 1×10⁵ B16 melanoma cells expressing OVA were subcutaneously ingested in a CD45.1/CD45.2 congenic mouse, and 5×10⁵ OT-2T cells were transferred after 1 day. 1 day. 4 days, and 7 days after OT-2T cell transfer. 50 μg of mRNA of Example 6B or mRNA of Reference Example 1B was mixed with an in vivo-jetRNA transfection reagent (Polyplus-transfection SA), the mixture was transferred from a tail vein of a recipient mouse, and the size of B16 melanoma cells was observed.

mRNA of Example 6B suppresses the proliferation of B16 melanoma cells.

### [Example 11B] Polynucleotide for Producing Antigen-Presenting Extracellular Vesicles Containing HLA Class II Molecules, Human T-Cell Costimulatory Molecules, and Human T-Cell Stimulatory Cytokines in Membrane

A pET-15b vector encoding each of HLADR-1sc-TPI1-hCD81, hCD80-hCD9, and hIL-12sc-MFGe8 was linearized, the linearized vector was purified using a FastGene Gel/PCR extraction kit (NIPPON Genetics Co., Ltd.), and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1:1 and then used as RNAs for preparing the antigen-presenting extracellular vesicles of Example 11B.

### Test Example 16B: Experiment on Differentiation of TPI-1-Specific Human CD4-Positive T Cells into Th1T Cells In Vitro by Polynucleotide for Producing Antigen-Presenting Extracellular Vesicles

PlatA cells were transfected with a TPI-1 peptide-specific TCR and a pMXs vector encoding a fluorescent protein Venus using polyethylenimine “Max” (manufactured by Polysciences Inc.). The medium was changed 3 to 12 hours after transfection, supernatant was collected 48 and 72 hours after transfection, and the supernatant was passed through a 0.22 μm filter to prepare retroviral supernatant expressing a TPI-1 specific TCR. Peripheral blood was collected from an HLA-DR1-positive recipient, peripheral blood mononuclear cells were separated using Ficol. 2.0×10⁶ peripheral blood mononuclear cells were suspended in 200 μL of an RPMI 1640 medium to which 10% fetal bovine serum. 50 μM 2-mercaptoethanol, and penicillin/streptomycin were added, human IL-2 and 25 μl of dynabeads were mixed so that the final concentration was 20 ng/mL, and stimulation was performed in a 6 well plate for 24 hours. The viral particles prepared above were infected with RetroNectin (TAKARA) as instructed by the manufacturer to prepare human T cells simultaneously expressing a TPI-1 peptide-specific TCR and fluorescent protein Venus. mRNA of Example 11B or Reference Example 1B was mixed with an in vivo-jetRNA transfection reagent (Polyplus-transfection SA), the mixture was added to the prepared human T cells, and culture was performed in a 96 well round bottom plate for 7 days.

### Results

The RNA of Example 11B can remarkably induce proliferation of antigen-specific CD4-positive T cells into Th1 cells in vitro.

### [Example 12B] Preparation of Polynucleotide Expressing MHC Class I Molecules, T-Cell Costimulatory: Molecules, and T-Cell Stimulatory Cytokines

PLAT-A cells were seeded in a cell culture dish and cultured in a Dulbecco's modified Eagle medium to which 2% fetal bovine serum and penicillin/streptomycin were added. In cells at about 50% confluence, a pET-15b vector encoding each of CD80-MFG-E8 and sc-Trimer-CD81-IL-2 was linearized, the linearized vector was purified using a FastGene Gel/PCR extraction kit (NIPPON Genetics Co., Ltd.) according to the manufacturer's instructions, and in vitro transcription, capping, and poly A addition were performed on the purified vector using T7 mScript Standard mRNA Production System (manufactured by CELLSCRIPT. LLC) according to the manufacturer's instructions. The synthesized mRNAs were mixed at 1:1 and then used as RNAs for preparing the antigen-presenting extracellular vesicles of Example 12B.

### Test Example 17B: Experiment for Evaluating Anti-Tumor Effect by Antigen-Presenting Extracellular Vesicles

In order to determine whether the antigen-presenting extracellular vesicles obtained by purification of a stable cell strain have an anti-tumor effect. 1×10⁵ EL-4 cells expressing OVA were subcutaneously ingested in a C57BL/6 mouse. 1 day, 4 days, and 7 days after EL-4 cell ingestion. 50 μg of RNA of Example 12B or RNA of Reference Example 1 was mixed with an in vivo-jetRNA transfection reagent (Polyplus-transfection SA), the mixture was transferred from a tail vein of a recipient mouse, and the size of EL-4 cells was observed.

The RNA of Example 12B can suppress proliferation of EL-4 cells.

Hereinabove, as described in the examples, the antigen-presenting cells and the polynucleotide for producing antigen-presenting extracellular vesicles described in the present specification can satisfactorily activate, proliferate, and/or differentiate antigen-specific T cells (for example, antigen-specific CD8-positive T cells, antigen-specific CD4-positive cells, and the like).

Hereinafter, a summary of the sequences included in the sequence listing is described.

TABLE 22

| | SEQ ID NO: | |
|---|---|---|
| | Amino acid | |
| | sequence | Polynucleotide |
| Signal peptide of β₂ | 1 | 2 |
| microglobulin | | |
| β₂ Microglobulin | 7 | 8 |
| (from which signal peptide | | |
| is removed) | | |
| MHC class I α chain | 9 | 10 |
| (from which signal peptide | | |
| is removed) | | |
| Signal peptide of MHC | 33 | 34 |
| class II β chain | | |
| MHC class II β chain | 37 | 38 |
| (from which signal peptide | | |
| is removed) | | |
| MHC class II α chain | 45 | 46 |
| (full length) | | |
| MHC class II α chain | 71 | 72 |
| (from which signal peptide | | |
| is removed) | | |

TABLE 23

| | SEQ ID NO: | |
|---|---|---|
| | Amino acid | |
| | sequence | Polynucleotide |
| OVA peptide 1 (for MHC | 3 | 4 |
| class I molecule) | | |
| OVA peptide 2 (for MHC | 35 | 36 |
| class II molecule) | | |

TABLE 24

| | SEQ ID NO: | |
|---|---|---|
| | Amino acid | |
| | sequence | Polynucleotide |
| CD9 (full length) | 21 | 22 |
| CD63 (full length) | 27 | 28 |
| CD63 | 57 | 58 |
| (partial sequence containing | | |
| TM1 to TM3) | | |
| CD63 | 59 | 60 |
| (partial sequence containing | | |
| TM4) | | |
| CD81 (full length) | 15 | 16 |
| CD81 | 61 | 62 |
| (partial sequence containing | | |
| TM1 to TM3) | | |
| CD81 | 63 | 64 |
| (partial sequence containing | | |
| TM4) | | |
| MFG-E8 | 49 | 50 |
| (from which signal peptide | | |
| is removed) | | |

TABLE 25

| | SEQ ID NO: | |
|---|---|---|
| | Amino acid | |
| | sequence | Polynucleotide |
| IL-2 | 25 | 26 |
| (from which signal peptide | | |
| is removed) | | |
| IL-4 | 53 | 54 |
| (from which signal peptide | | |
| is removed) | | |
| TGF-81 (full length) | 47 | 48 |
| TGF-81 | 73 | 74 |
| (from which signal peptide | | |
| is removed) | | |

TABLE 26

| | SEQ ID NO: | |
|---|---|---|
| | Amino acid | |
| | sequence | Polynucleotide |
| CD80 (full length) | 19 | 20 |
| CD80 | 67 | 68 |
| (from which signal | | |
| peptide is removed) | | |

TABLE 27

| | SEQ ID NO: | |
|---|---|---|
| | Amino acid | |
| | sequence | Polynucleotide |
| Peptide linker 1 | 5 | 6 |
| Peptide linker 2 | 11 | 12 |
| Peptide linker 3 | 29 | 30 |
| Peptide linker 4 | 39 | 40 |
| Peptide linker 5 | 77 | 78 |

TABLE 28

| | SEQ ID NO: | |
|---|---|---|
| | Amino acid | |
| | sequence | Polynucleotide |
| Single chain MHC class I | 65 | 66 |
| molecule | | |
| (β₂ microglobulin + | | |
| MHC class I α chain) | | |
| sc-Trimer | 13 | 14 |
| (OVA peptide 1 + | | |
| single chain MHC class I | | |
| molecule) | | |
| sc-Trimer + | 17 | 18 |
| CD 8 1 (full length) | | |
| sc-Dimer | 41 | 42 |
| (OVA peptide 2 + | | |
| MHC class II β chain) | | |
| sc-Dimer + | 43 | 44 |
| CD81 (full length) | | |
| CD63-IL-2 | 31 | 32 |
| CD81-IL-4 | 55 | 56 |
| TGF-β1 (full length) + | 51 | 52 |
| MGF-E8 (from which | | |
| signal peptide is removed) | | |
| TGF-β1 (from which | 75 | 76 |
| signal peptide is removed) + | | |
| MGF-E8 (from which | | |
| signal peptide is removed) | | |
| CD80 (full length) + | 23 | 24 |
| CD9 (full length) | | |
| CD80 (from which signal | 69 | 70 |
| peptide is removed) + | | |
| CD9 (full length) | | |

TABLE 29

| | SEQ ID NO: | |
|---|---|---|
| | Amino acid | |
| | sequence | Polynucleotide |
| PNE tag | 79 | 80 |
| Anti-PNE tag nanobody | 81 | 82 |
| (full length) | | |
| Anti-PNE tag nanobody | 83 | 84 |
| (from which signal peptide | | |
| is removed) | | |
| CD8a | 85 | 86 |
| Anti-PNE tag nanobody | 87 | 88 |
| (full length) + CD8a + | | |
| CD81 (full length) | | |
| Anti-PNE tag nanobody | 89 | 90 |
| (from which signal peptide | | |
| is removed) + CD8a + | | |
| CD81 (full length) | | |

TABLE 30-1

| | SEQ ID NO: | |
|---|---|---|
| | Amino acid | |
| | sequence | Polynucleotide |
| sc-Dimer-CD81-IL-12α | 91 | 92 |
| IL-12α | 93 | 94 |
| (from which signal peptide is removed) | | |
| CD81-IL-12α | 95 | 96 |
| IL-12β | 97 | 98 |
| IL-6 | 99 | 100 |
| (from which signal peptide is removed) | | |
| CD81-IL-6 | 101 | 102 |
| hCD80 | 103 | 104 |
| hCD9 | 105 | 106 |
| hCD80-hCD9 | 107 | 108 |
| hIL-2 | 109 | 110 |
| (from which signal peptide is removed) | | |
| hCD63 | 111 | 112 |
| (amino acids 1 to 170) | | |
| hCD63 | 113 | 114 |
| (amino acids 171 to 238) | | |
| hCD63-IL-2 | 115 | 116 |
| Signal peptide of hβ2 microglobulin | 117 | 118 |
| WT1 PEPTIDE 1 | 119 | 120 |
| (for MHC class I molecule) | | |
| hβ2 Microglobulin | 121 | 122 |
| (from which signal peptide is removed) | | |
| hMHC class I α chain | 123 | 124 |
| (from which signal peptide is removed) | | |
| h single chain MHC class I molecule | 125 | 126 |
| (β2 microglobulin (from which signal peptide | | |
| is removed) + peptide linker 2 + MHC class | | |
| I α chain (from which signal peptide is | | |
| removed)) | | |
| hsc-Trimer | 127 | 128 |
| (WT1 peptide 1 + peptide linker 1 + single | | |
| chain MHC class I molecule) | | |

TABLE 30-2

| | | |
|---|---|---|
| hCD81 | 129 | 130 |
| hsc-Trimer − CD81 | 131 | 132 |
| (sc-Trimer + CD81) | | |
| CD81 − IL-2 | 133 | 134 |
| sc-Trimer − CD81 − IL-2 | 135 | 136 |
| (sc-Trimer + CD81) | | |
| Aka-Luc | 137 | 138 |
| CD63 − Aka-Luc | 139 | 140 |
| SARS-CoV2 peptide 1 | 141 | 142 |
| hMHC class I (HLA-A0201) c chain | 143 | 144 |
| (from which signal peptide is removed) | | |
| h single chain MHC class I molecule | 145 | 146 |
| (β2 microglobulin (from which signal peptide | | |
| is removed) + peptide linker 2 + MHC class | | |
| I (HLA-A0201) a chain (from which signal | | |
| peptide is removed)) | | |
| hsc-Trimer | 147 | 148 |
| (SARS-CoV2 peptide 1 + peptide linker 1 + | | |
| single chain MHC class I (HLA-A0201) | | |
| molecule) | | |
| hsc-Trimer − CD81 | 149 | 150 |
| (SARS-CoV2sc − Trimer + CD81) | | |

TABLE 31

| | SEQ ID NO: | |
|---|---|---|
| | Amino acid | |
| | sequence | Polynucleotide |
| HLADR1β chain signal sequence | 151 | 152 |
| TPI peptide | 153 | 154 |
| Linker | 155 | 156 |
| HLADR1β chain | 157 | 158 |
| hCD81 | 159 | 160 |
| P2A | 161 | 162 |
| HLADR1α chain | 163 | 164 |
| HLADR-1sc-TPI1-hCD81 | 165 | 166 |
| IL-12B | 167 | 168 |
| Linker | 169 | 170 |
| IL-12α (no signal sequence) | 171 | 172 |
| Linker | 173 | 174 |
| MFGE8 (no signal sequence) | 175 | 176 |
| hIL-12sc-MFGe8 | 177 | 178 |
| TCRβ chain | 179 | 180 |
| P2A | 181 | 182 |
| TCRα chain | 183 | 184 |
| P2A | 185 | 186 |
| Venus | 187 | 188 |
| Fusion protein of TPI-1 peptide-specific | 189 | 190 |
| TCR and Venus | | |
| sc-Trimer | 191 | 192 |
| T2A | 193 | 194 |
| IL-2 | 195 | 196 |
| Linker | 197 | 198 |
| CD8 (partial sequence) | 199 | 200 |
| P2A | 201 | 202 |
| CD80 | 203 | 204 |
| sc-Trimer-T2A-IL-2-CD8-P2A-CD80 | 205 | 206 |
| CD81 | 207 | 208 |
| OVA | 209 | 210 |

TABLE 32

| | SEQ ID NO: | |
|---|---|---|
| | Amino acid | |
| | sequence | Polynucleotide |
| T2A | 211 | — |
| P2A | 212 | — |
| E2A | 213 | — |
| F2A | 214 | — |

TABLE 33

| | SEQ ID NO: | |
|---|---|---|
| | Amino acid | |
| | sequence | Polynucleotide |
| Signal peptide of β2 microglobulin | 215 | 216 |
| OVA peptide | 217 | 218 |
| Linker | 219 | 220 |
| β2 microglobulin (from which signal | 221 | 222 |
| peptide is removed) | | |
| Linker | 223 | 224 |
| MHC class Iα chain | 225 | 226 |
| (from which signal peptide is removed) | | |
| T2A | 227 | 228 |
| TfR (transferrin receptor) | 229 | 230 |
| Linker | 231 | 232 |
| IL-15Rα sushi | 233 | 234 |
| Linker | 235 | 236 |
| IL-15 | 237 | 238 |
| FLAG | 239 | 240 |
| P2A | 241 | 242 |
| CD80 | 243 | 244 |
| OVApscMHCI-T2A-TfR-IL-15Ra | 245 | 246 |
| sushi-linker-IL15-Flag-P2A-CD80 | | |

TABLE 34

| | SEQ ID NO: | |
|---|---|---|
| | Amino acid | |
| | sequence | Polynucleotide |
| Signal peptide of MHC class IIβ chain | 247 | 248 |
| OVA peptide | 249 | 250 |
| Linker | 251 | 252 |
| MHC class IIβ chain (from which signal | 253 | 254 |
| peptide is removed) | | |
| P2A | 255 | 256 |
| MHC class IIα chain | 257 | 258 |
| T2A | 259 | 260 |
| IL-12β | 261 | 262 |
| Linker | 263 | 264 |
| IL-12α | 265 | 266 |
| Linker | 267 | 268 |
| FLAG | 269 | 270 |
| Transmembrane domain of CD8 | 271 | 272 |
| P2A | 273 | 274 |
| CD80 | 275 | 276 |
| OVAp-MHCIIβ-P2A-MHCIIα-T2A-IL-12 | 277 | 278 |
| single chain-CD8-P2A-CD80 | | |

TABLE 35

| | SEQ ID NO: | |
|---|---|---|
| | Amino acid | |
| | sequence | Polynucleotide |
| Gtf2i peptide | 279 | 280 |
| sc-Trimer(Gtf2i)-T2A-IL-2-CD8-P2A-CD80 | 281 | 282 |
| RPL18 peptide | 283 | 284 |

1. A polynucleotide comprising at least one sequence selected from the group consisting of:
(a) a sequence encoding a fusion protein (A) which comprises an antigen-presenting MHC molecule and is capable of presenting the antigen-presenting MHC molecule outside membrane of a cell or an extracellular vesicle:
(b) a sequence encoding a fusion protein (B) which comprises at least one T-cell stimulatory cytokine or subunit thereof and is capable of presenting the T-cell stimulatory cytokine outside membrane of a cell or an extracellular vesicle:
(c) a sequence encoding a fusion protein (C) which comprises a T-cell costimulatory molecule and is capable of presenting the T-cell costimulatory molecule outside membrane of a cell or an extracellular vesicle:
(d) a sequence encoding a fusion protein (D) which comprises an antigen-presenting MHC molecule and at least one T-cell stimulatory cytokine or subunit thereof and is capable of presenting the antigen and the T-cell stimulatory cytokine outside membrane of a cell or an extracellular vesicle; and
(e) a sequence encoding a fusion protein (E) which comprises an antigen-presenting MHC molecule, at least one T-cell stimulatory cytokine or subunit thereof, and a T-cell costimulatory molecule, and is capable of presenting the antigen, the T-cell stimulatory cytokine, and the T-cell costimulatory molecule outside membrane of a cell or an extracellular vesicle.

2. The polynucleotide according to claim 1, wherein the fusion protein defined as (A) comprises an antigen-presenting MHC molecule, and
a membrane protein capable of being expressed in membrane of a cell or an extracellular vesicle or a transmembrane domain thereof, or
a protein capable of binding to membrane of a cell or an extracellular vesicle or a membrane-binding domain thereof.

3. The polynucleotide according to claim 1, wherein the fusion protein defined as (A) comprises
an antigen-presenting MHC molecule, and
a Tetraspanin or a transmembrane domain thereof or MFG-E8 or a membrane-binding domain thereof.

4. The polynucleotide according to claim 1, wherein the fusion protein defined as (A) comprises an antigen-presenting MHC molecule containing a transmembrane domain.

5. The polynucleotide according to claim 1, wherein the fusion protein defined as (A) comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of a single chain MHC molecule,
(A-4) a spacer sequence which may be optionally present, and
(A-5) an amino acid sequence of a Tetraspanin or a transmembrane domain thereof, in this order.

6. The polynucleotide according to claim 1, wherein the fusion protein defined as (A) comprises an amino acid sequence containing, from an N-terminal side thereof,
(A-1) an amino acid sequence of an MHC molecule-restricted antigen peptide,
(A-2) a spacer sequence which may be optionally present,
(A-3) an amino acid sequence of an MHC class Iα chain, β₂ microglobulin, an MHC class IIα chain, or an MHC class IIβ chain,
(A-4) a spacer sequence which may be optionally present, and
(A-5) an amino acid sequence of a Tetraspanin or a transmembrane domain thereof, in this order.

7. The polynucleotide according to claim 6, wherein (A-6) a sequence encoding an amino acid sequence of β₂ microglobulin, an MHC class Iα chain, an MHC class IIβ chain, or an MHC class IIα chain is further comprised.

8. The polynucleotide according to claim 1, wherein the fusion protein defined as (A) comprises an amino acid sequence containing, from an N-terminal side thereof,
(A-1) an amino acid sequence of an MHC class I molecule-restricted antigen peptide,
(A-2) a spacer sequence which may be optionally present,
(A-3) an amino acid sequence of a single chain MHC class I molecule,
(A-4) a spacer sequence which may be optionally present, and
(A-5) an amino acid sequence of a Tetraspanin or a transmembrane domain thereof, in this order.

9. The polynucleotide according to claim 1, wherein the fusion protein defined as (A) comprises an amino acid sequence containing, from an N-terminal side thereof,
(A-1) an amino acid sequence of an MHC class II molecule-restricted antigen peptide,
(A-2) a spacer sequence which may be optionally present,
(A-3) an amino acid sequence of an MHC class IIβ chain,
(A-4) a spacer sequence which may be optionally present, and
(A-5) an amino acid sequence of a Tetraspanin or a transmembrane domain thereof, in this order.

10. The polynucleotide according to claim 9, wherein (A-6) a sequence encoding an amino acid sequence of an MHC class IIα chain is further comprised.

11. The polynucleotide according to claim 1, wherein the fusion protein defined as (A) comprises an amino acid sequence containing, from an N-terminal side thereof,
(A-1) an amino acid sequence of an MHC molecule-restricted antigen peptide,
(A-2) a spacer sequence which may be optionally present, and
(A-3) an amino acid sequence of a single chain MHC molecule containing a transmembrane domain, in this order.

12. The polynucleotide according to claim 1, wherein the fusion protein defined as (A) comprises an amino acid sequence containing, from an N-terminal side thereof,
(A-1) an amino acid sequence of an MHC molecule-restricted antigen peptide,
(A-2) a spacer sequence which may be optionally present, and
(A-3) an amino acid sequence of an MHC class Iα chain, a fusion protein of β₂ microglobulin and an MHC class Iα chain, an MHC class IIα chain, or an MHC class IIβ chain, in this order.

13. The polynucleotide according to claim 12, wherein (A-6) a sequence encoding an amino acid sequence of β₂ microglobulin, an MHC class IIβ chain, or an MHC class IIα chain is further comprised.

14. The polynucleotide according to claim 1, wherein the fusion protein defined as (A) comprises an amino acid sequence containing, from an N-terminal side thereof,
(A-1) an amino acid sequence of an MHC class I molecule-restricted antigen peptide,
(A-2) a spacer sequence which may be optionally present, and
(A-3) an amino acid sequence of a single chain MHC class I molecule containing a transmembrane domain, in this order.

15. The polynucleotide according to claim 1, wherein the fusion protein defined as (A) comprises an amino acid sequence containing, from an N-terminal side thereof,
(A-1) an amino acid sequence of an MHC class II molecule-restricted antigen peptide,
(A-2) a spacer sequence which may be optionally present, and
(A-3) an amino acid sequence of an MHC class IIβ chain, in this order.

16. The polynucleotide according to claim 15, wherein a sequence encoding an amino acid sequence of an MHC class IIα chain is further comprised.

17. The polynucleotide according to claim 1, wherein the fusion protein defined as (B) comprises at least one T-cell stimulatory cytokine or subunit thereof, and
a membrane protein capable of being expressed in membrane of a cell or an extracellular vesicle or a transmembrane domain thereof, or
a protein capable of binding to membrane of a cell or an extracellular vesicle or a domain thereof.

18. The polynucleotide according to claim 1, wherein the fusion protein defined as (B) comprises at least one T-cell stimulatory cytokine or subunit thereof and a partial sequence of a Tetraspanin, the partial sequence of the Tetraspanin contains at least two transmembrane domains, and the at least one T-cell stimulatory cytokine is disposed between the two transmembrane domains.

19. The polynucleotide according to claim 1, wherein the fusion protein defined as (B) comprises at least one T-cell stimulatory cytokine or subunit thereof and MFG-E8 or a membrane-binding domain thereof.

20. The polynucleotide according to claim 1, wherein the fusion protein defined as (B) comprises an amino acid sequence containing, from an N-terminal side thereof,
(B-1) a partial sequence of a Tetraspanin containing, from an N-terminal side thereof, a transmembrane domain 1, a small extracellular loop, a transmembrane domain 2, a small intracellular loop, and a transmembrane domain 3,
(B-2) a spacer sequence which may be optionally present,
(B-3) an amino acid sequence of a first T-cell stimulatory cytokine,
(B-4) a spacer sequence which may be optionally present, and
(B-5) a partial sequence of a Tetraspanin containing a transmembrane domain 4, in this order.

21. The polynucleotide according to claim 1, wherein the fusion protein defined as (B) comprises an amino acid sequence containing, from an N-terminal side thereof,
(B-3) an amino acid sequence of a first T-cell stimulatory cytokine,
(B-4) a spacer sequence which may be optionally present, and
(B-5) an amino acid sequence of MFG-E8 or a membrane-binding domain thereof, in this order.

22. The polynucleotide according to claim 1, wherein the fusion protein defined as (B) comprises at least one T-cell stimulatory cytokine or subunit thereof and CD8 or a transmembrane domain thereof.

23. The polynucleotide according to claim 1, wherein the fusion protein defined as (B) comprises an amino acid sequence containing, from an N-terminal side thereof,
(B-3) an amino acid sequence of a first T-cell stimulatory cytokine,
(B-4) a spacer sequence which may be optionally present, and
(B-5) an amino acid sequence of CD8 or a transmembrane domain thereof, in this order.

24. The polynucleotide according to claim 1, wherein the T-cell stimulatory cytokine is IL-2, IL-4, IL-6, IL-12, a subunit of IL-12, IL-15, or TGF-β.

25. The polynucleotide according to claim 1, wherein the fusion protein defined as (C) comprises a T-cell costimulatory molecule, and
a membrane protein capable of being expressed in membrane of a cell or an extracellular vesicle or a transmembrane domain thereof, or
a protein capable of binding to membrane of a cell or an extracellular vesicle or a membrane-binding domain thereof.

26. The polynucleotide according to claim 1, wherein the fusion protein defined as (C) comprises a T-cell costimulatory molecule, and
a Tetraspanin or a transmembrane domain thereof, or
MFG-E8 or a membrane-binding domain thereof.

27. The polynucleotide according to claim 1, wherein the fusion protein defined as (C) comprises a T-cell costimulatory molecule containing a transmembrane domain.

28. The polynucleotide according to claim 1, wherein the fusion protein defined as (C) comprises an amino acid sequence containing, from an N-terminal side thereof,
(C-1) an amino acid sequence of a T-cell costimulatory molecule,
(C-2) a spacer sequence which may be optionally present, and
(C-3) an amino acid sequence of a Tetraspanin or a transmembrane domain thereof, in this order.

29. The polynucleotide according to claim 1, wherein the fusion protein defined as (D) comprises the antigen-presenting MHC molecule,
the at least one T-cell stimulatory cytokine or subunit thereof, and
a membrane protein capable of being localized to membrane of a cell or an extracellular vesicle or a transmembrane domain thereof, or
a protein capable of binding to membrane of a cell or an extracellular vesicle or a membrane-binding domain thereof.

30. The polynucleotide according to claim 29, wherein the membrane protein capable of being localized to membrane of an extracellular vesicle or the protein capable of binding to membrane of an extracellular vesicle is a Tetraspanin or MFG-E8.

31. The polynucleotide according to claim 29, wherein the membrane protein capable of being localized to membrane of a cell or the protein capable of binding to membrane of a cell is CD8.

32. The polynucleotide according to claim 1, wherein the fusion protein defined as (D) comprises an amino acid sequence containing, from an N-terminal side thereof,
(D-1) an amino acid sequence of an MHC molecule-restricted antigen peptide,
(D-2) a spacer sequence which may be optionally present,
(D-3) an amino acid sequence of a single chain MHC molecule,
(D-4) a spacer sequence which may be optionally present, and
(D-5) an amino acid sequence of a fusion peptide comprising a Tetraspanin or a transmembrane domain thereof or MFG-E8 or a membrane-binding domain thereof, and the at least one T-cell stimulatory cytokine or subunit thereof, in this order.

33. The polynucleotide according to claim 1, wherein the fusion protein defined as (D) comprises an amino acid sequence containing, from an N-terminal side thereof,
(D-1) an amino acid sequence of a fusion peptide comprising a Tetraspanin or a transmembrane domain thereof or MFG-E8 or a membrane-binding domain thereof, and the at least one T-cell stimulatory cytokine or subunit thereof,
(D-2) a spacer sequence which may be optionally present,
(D-3) an amino acid sequence of a single chain MHC molecule,
(D-4) a spacer sequence which may be optionally present, and
(D-5) an amino acid sequence of an MHC molecule-restricted antigen peptide, in this order.

34. The polynucleotide according to claim 32, wherein the fusion peptide comprising a Tetraspanin or a transmembrane domain thereof or MFG-E8 or a membrane-binding domain thereof, and the at least one T-cell stimulatory cytokine or subunit thereof comprises an amino acid sequence containing, from an N-terminal side thereof,
(1) a partial sequence of a Tetraspanin containing a transmembrane domain 1, a small extracellular loop, a transmembrane domain 2, a small intracellular loop, and a transmembrane domain 3,
(2) a spacer sequence which may be optionally present,
(3) an amino acid sequence of the at least one T-cell stimulatory cytokine or subunit thereof,
(4) a spacer sequence which may be optionally present, and
(5) a partial sequence of a Tetraspanin containing a transmembrane domain 4, in this order.

35. The polynucleotide according to claim 32, wherein the fusion peptide comprising a Tetraspanin or a transmembrane domain thereof or MFG-E8 or a membrane-binding domain thereof, and the at least one T-cell stimulatory cytokine or subunit thereof comprises an amino acid sequence containing, from an N-terminal side thereof,
(1) an amino acid sequence of the at least one T-cell stimulatory cytokine or subunit thereof,
(2) a spacer sequence which may be optionally present, and
(3) an amino acid sequence of MFG-E8 or a membrane-binding domain thereof, in this order.

36. The polynucleotide according to claim 1, wherein the fusion protein defined as (D) comprises the antigen-presenting MHC molecule, the at least one T-cell stimulatory cytokine or subunit thereof, and a membrane protein capable of being localized to membrane of a cell or a transmembrane domain thereof or a protein capable of binding to membrane of a cell or a membrane-binding domain thereof.

37. The polynucleotide according to claim 32, wherein the MHC molecule-restricted antigen peptide is an MHC class I molecule-restricted antigen peptide, and the single chain MHC molecule contains an extracellular domain of an MHC class Iα chain.

38. The polynucleotide according to claim 32, wherein the MHC molecule-restricted antigen peptide is an MHC class II molecule-restricted antigen peptide, and the single chain MHC molecule contains an extracellular domain of an MHC class IIα chain and/or an extracellular domain of an MHC class IIβ chain.

39. The polynucleotide according to claim 1, comprising the sequence defined as (a) and the sequence defined as (b).

40. The polynucleotide according to claim 39, wherein the polynucleotide encodes an amino acid sequence in which the fusion protein (A) and the fusion protein (B) are fused.

41. The polynucleotide according to claim 40, wherein the polynucleotide encodes an amino acid sequence in which the fusion protein (A) and the fusion protein (B) are fused through at least one 2A peptide.

42. The polynucleotide according to claim 39, further comprising the sequence defined as (c).

43. The polynucleotide according to claim 42, wherein the polynucleotide encodes an amino acid sequence in which the fusion protein (A), the fusion protein (B), and the fusion protein (C) are fused.

44. The polynucleotide according to claim 41, wherein the polynucleotide encodes an amino acid sequence in which the fusion protein (A), the fusion protein (B), and the fusion protein (C) are each fused through at least one independent 2A peptide.

45. The polynucleotide according to claim 44, wherein the polynucleotide comprises, from a 5′ end:
the sequence defined as (a);
a sequence encoding at least one first 2A peptide;
the sequence defined as (b);
a sequence encoding at least one second 2A peptide; and
the sequence defined as (c), in this order.

46. The polynucleotide according to claim 1, comprising the sequence defined as (d).

47. The polynucleotide according to claim 46, further comprising the sequence defined as (c).

48. The polynucleotide according to claim 47, wherein the polynucleotide encodes an amino acid sequence in which the fusion protein (D) and the fusion protein (C) are fused.

49. The polynucleotide according to claim 48, wherein the polynucleotide encodes an amino acid sequence in which the fusion protein (D) and the fusion protein (C) are fused through at least one 2A peptide.

50. The polynucleotide according to claim 1, comprising the sequence defined as (e).

51. A vector comprising the polynucleotide according to claim 1.

52. A pharmaceutical composition comprising the polynucleotide according to claim 1 or a vector comprising the polynucleotide, and a pharmacologically acceptable carrier.

53. A pharmaceutical composition for treating or preventing an infectious disease, comprising the polynucleotide according to claim 1 or a vector comprising the polynucleotide, and a pharmacologically acceptable carrier.

54. A pharmaceutical composition for treating or preventing cancer, comprising the polynucleotide according to claim 1 or a vector comprising the polynucleotide.

55. A pharmaceutical composition for treating or preventing an autoimmune disease, comprising the polynucleotide according to claim 1 or a vector comprising the polynucleotide.

56. A pharmaceutical composition for treating or preventing an allergic disease, comprising the polynucleotide according to claim 1 or a vector comprising the polynucleotide.

57. A method for activating and/or proliferating T cells against a specific antigen, the method comprising: introducing the polynucleotide according to claim 1 or a vector comprising the polynucleotide to cells in vitro or ex vivo to produce antigen-presenting cells and/or antigen-presenting extracellular vesicles; and contacting the produced antigen-presenting cells and/or antigen-presenting extracellular vesicles with T cells in vitro or ex vivo.
